(19) United States
(12) Patent Application Publication
(10) Pub. No.: US 2026/0193348 A1
(43) Pub. Date: Jul. 9, 2026

(54) NOVEL ANTIBODIES TARGETING CD3 AND ANOTHER TARGET AND USES THEREOF

(71) Applicant: ANTENGENE BIOLOGICS LIMITED, Shanghai (CN)

(72) Inventors: Bing HOU, Shanghai (CN); Hui YUWEN, Shanghai (CN); Tengteng LI, Shanghai (CN); Yijing REN, Shanghai (CN); Ao SUN, Shanghai (CN); Gang BIAN, Shanghai (CN); Peng CHEN, Shanghai (CN); Huiling LIU, Shanghai (CN); Bo SHAN, Shanghai (CN)

(21) Appl. No.: 18/863,998

(22) PCT Filed: Jun. 7, 2024
(86) PCT No.: PCT/CN2024/097996

§ 371 Date: Nov. 7, 2024

(30) Foreign Application Priority Data
Jun. 9, 2023 (WO) PCT/CN2023/099556
Sep. 13, 2023 (WO) PCT/CN2023/118666
Apr. 5, 2024 (WO) PCT/CN2024/086269

(51) Int. Cl. C07K 16/28; A61K 39/00; A61K 45/06; A61P 35/00; A61P 37/00; C07K 16/30; G01N 33/564; G01N 33/575

(52) U.S. Cl. CPC C07K 16/2809; A61K 45/06; A61P 35/00; A61P 37/00; C07K 16/28; C07K 16/2803; C07K 16/2887; C07K 16/3084; G01N 33/564; G01N 33/5758; A61K 2039/505; C07K 2317/24; C07K 2317/31; C07K 2317/55; C07K 2317/622; C07K 2317/73; C07K 2317/92; G01N 2333/7051

(57) ABSTRACT

The present disclosure provides novel antibodies targeting CD3 and one or more other targets, or antigen-binding fragments thereof, isolated polynucleotides encoding the same, pharmaceutical composition comprising the same and the uses thereof.

### FIELD OF THE INVENTION

The present disclosure generally relates to novel antibodies targeting CD3 and one or more other targets, antigen-binding fragments thereof, and uses of the same.

### BACKGROUND

The CD3 (cluster of differentiation 3) T-cell co-receptor is a protein complex and is composed of four distinct chains, a CD3gamma chain, a CD3delta chain, and two CD3epsilon chains. These chains associate with a molecule known as the T-cell receptor (TCR) and the zeta-chain to generate activation signal in T lymphocytes. The TCR, zeta-chain, and CD3 molecules together form the TCR-CD3 complex, in which TCR as a subunit recognizes and binds to antigen, and CD3 as a subunit transfers and conveys the antigen-stimulation to signaling pathway, and ultimately regulates T-cell activity. The CD3 protein is virtually present in all T cells.

Mouse monoclonal antibodies specific for human CD3, such as OKT3 (Kung et al., (1979) Science 206:347-9), were the first generation CD3 antibodies for treatment. Although OKT3 has strong immunosuppressive potency, its clinical use was hampered by serious side effects linked to its immunogenic and mitogenic potentials (Chatenoud (2003) Nature Reviews Immunology 3:123-132). OKT3 induced an anti-globulin response, promoting its own rapid clearance and neutralization (Chatenoud et al., (1982) Eur. J. Immunol. 137:830-8). In addition, OKT3 induced T-cell proliferation and cytokine production in vitro, and led to a large scale release of cytokine in vivo (Hirsch et al., (1989) J. Immunol 142:737-43). The cytokine release (also referred to as “cytokine storm”) in turn led to a “flu-like” syndrome, characterized by fever, chills, headaches, nausea, vomiting, diarrhea, respiratory distress, septic meningitis and hypotension (Chatenoud (2003) Nature Reviews Immunology 3:123-132). Such serious side effects limited the more widespread use of OKT3 in transplantation as well as the extension of its use to other clinical fields such as autoimmunity.

A more recent application of CD3 antibodies is in the form of bispecific antibodies, binding CD3 on the one hand and a tumor cell antigen on the other hand. The simultaneous binding of such an antibody to both of its targets will force a temporary interaction between target cell and T cell, causing activation of any cytotoxic T cell and subsequent lysis of the target cell.

Needs remain for novel antibodies that targeting CD3 and one or more other targets.

### SUMMARY OF THE INVENTION

In one aspect, the present disclosure provides an antibody or antigen-binding fragment thereof, comprising:
i. a first binding moiety that binds to a conformational epitope of CD3, and
ii. a second binding moiety that binds to a second target other than CD3,
   wherein the first binding moiety and the second binding moiety are independently a single-chain Fv (scFv) or a Fab domain.

In one aspect, the present disclosure provides an antibody or antigen-binding fragment thereof, comprising:
i. a first binding moiety that binds to CD3, which comprises:
   one, two or three heavy chain complementarity determining regions (HCDR1, HCDR2 and/or HCDR3) contained within SEQ ID NOs: 3, 7, 11, 21, 22, 23, 26, 27, 28, 29, 30, 80, 82, 83, 84, 85, 86, 119, 120, 122, 124, 125, 126, 128, 131, 134, 136, 138, 139, 145, 195, 196, 197, 200, 201, 202, 203, 204, 205, 207, 208, 209, 210, 211, 221, 222, 223, 224, 225, 226, 227, 228, 229, 230, 231, 232 or 315; and/or
   one, two or three light chain complementarity determining regions (LCDR1, LCDR2 and/or LCDR3) contained within SEQ ID NOs: 4, 8, 12, 24, 25, 81, 87, 88, 89, 90, 91, 92, 93, 94, 95, 141, 146, 198, 199, 206, 212, 213, 214, 215, 216, 217, 218, 219, 220, 233, 234 or 316;
ii. a second binding moiety that binds to a second target other than CD3,
   wherein the first binding moiety and the second binding moiety are independently a single-chain Fv (scFv) or a Fab domain.

In some embodiments, the first binding moiety is an anti-CD3 scFv domain, and the second binding moiety is a Fab domain; or the first binding moiety is an anti-CD3 Fab domain, and the second binding moiety is a scFv domain.

In some embodiments, the first binding moiety is linked to the second binding moiety directly or via a linker.

In some embodiments, the N-terminus of the first binding moiety is linked to the C-terminus of the second binding moiety via the linker.

In some embodiments, the antibody or antigen-binding fragment thereof provided herein comprises, from N-terminus to C-terminus, a first chain comprising a VH region from the second binding moiety, a CH1 region, a VH region from the first binding moiety and a VL region from the first binding moiety.

In some embodiments, the VH region from the first binding moiety and the VL region from the first binding moiety are directly linked or linked via a linker.

In some embodiments, the antibody or antigen-binding fragment thereof provided herein further comprises a second chain comprising a VL region from the second binding moiety and a CL region.

In some embodiments, the antibody or antigen-binding fragment thereof provided herein further comprises a third binding moiety that binds to a third target other than CD3. In some embodiments, the second target is the same as the third target. In some embodiments, the third binding moiety is a Fab domain.

In some embodiments, the first binding moiety comprises one or two or three of HCDR1, HCDR2 and HCDR3 comprising an amino acid sequence selected from the group consisting of SEQ ID NOs: 31~40, 44, 45, 46, 50, 51, 52, 96~106, 121, 123, 127, 129, 130, 132, 133, 135, 137, 140, 143, 144, 182, 183, 184, 307, 308 and 309.

In some embodiments, the first binding moiety comprises one or two or three of LCDR1, LCDR2 and LCDR3 comprising an amino acid sequence selected from the group consisting of SEQ ID NOs: 41, 42, 43, 47, 48, 49, 53, 54, 55, 107~118, 142, 149, 185, 186, 187, 310, 311 and 312.

In some embodiments, the first binding moiety comprises:
a HCDR1 comprising an amino acid sequence selected from the group consisting of SEQ ID NOs: 31, 44, 50, 96, 97, 98, 132, 140, 182 and 307;
a HCDR2 comprising an amino acid sequence selected from the group consisting of SEQ ID NOs: 32, 33, 34, 45, 51, 99, 100, 101, 102, 121, 123, 129, 143, 183 and 308; and
a HCDR3 comprising an amino acid sequence selected from the group consisting of SEQ ID NOs: 35, 36, 37, 38, 39, 40, 46, 52, 103, 104, 105, 106, 127, 130, 133, 135, 137, 144, 184 and 309.

In some embodiments, the first binding moiety comprises:
a LCDR1 comprising an amino acid sequence selected from the group consisting of SEQ ID NOs: 41, 47, 53, 107, 108, 109, 110, 111, 112, 113, 114, 115, 116, 185 and 310;
a LCDR2 comprising an amino acid sequence selected from the group consisting of SEQ ID NOs: 42, 48, 54, 117, 186 and 311; and
a LCDR3 comprising an amino acid sequence selected from the group consisting of SEQ ID NOs: 43, 49, 55, 118, 142, 149, 187 and 312.

In some embodiments, the first binding moiety comprises a VH region having an amino acid sequence as set forth in SEQ ID NOs: 3, 7, 11, 21, 22, 23, 26, 27, 28, 29, 30, 80, 82, 83, 84, 85, 86, 119, 120, 122, 124, 125, 126, 128, 131, 134, 136, 138, 139, 145, 195, 196, 197, 200, 201, 202, 203, 204, 205, 207, 208, 209, 210, 211, 221, 222, 223, 224, 225, 226, 227, 228, 229, 230, 231, 232 or 315, or a homologous sequence thereof having at least 80% sequence identity to SEQ ID NOs: 3, 7, 11, 21, 22, 23, 26, 27, 28, 29, 30, 80, 82, 83, 84, 85, 86, 119, 120, 122, 124, 125, 126, 128, 131, 134, 136, 138, 139, 145, 195, 196, 197, 200, 201, 202, 203, 204, 205, 207, 208, 209, 210, 211, 221, 222, 223, 224, 225, 226, 227, 228, 229, 230, 231, 232 or 315.

In some embodiments, the first binding moiety comprises a VL region having an amino acid sequence as set forth in SEQ ID NOs: 4, 8, 12, 24, 25, 81, 87, 88, 89, 90, 91, 92, 93, 94, 95, 141, 146, 198, 199, 206, 212, 213, 214, 215, 216, 217, 218, 219, 220, 233, 234 or 316, or a homologous sequence thereof having at least 80% sequence identity to SEQ ID NOs: 4, 8, 12, 24, 25, 81, 87, 88, 89, 90, 91, 92, 93, 94, 95, 141, 146, 198, 199, 206, 212, 213, 214, 215, 216, 217, 218, 219, 220, 233, 234 or 316.

In some embodiments, the first binding moiety comprises a VH/VL amino acid sequence pair selected from the group consisting of SEQ ID NOs: 3/4, 7/8, 11/12, 21/24, 22/24, 23/24, 21/25, 26/24, 27/24, 28/24, 29/24, 30/24, 80/81, 83/87, 84/87, 85/87, 86/87, 82/88, 82/89, 82/90, 82/91, 82/92, 82/93, 82/94, 82/95, 119/198, 120/198, 122/198, 124/198, 125/198, 126/198, 128/198, 131/198, 134/198, 136/198, 138/198, 139/198, 128/141, 195/198, 196/198, 197/198, 195/199, 200/198, 201/198, 202/198, 203/198, 204/198, 205/206, 208/212, 209/212, 210/212, 211/212, 207/213, 207/214, 207/215, 207/216, 207/217, 207/218, 207/219, 207/220, 221/233, 222/233, 223/233, 224/233, 225/233, 226/233, 227/233, 227/234, 228/233, 229/233, 230/233, 231/233, 232/233 and 315/316.

In some embodiments, the first binding moiety further comprises one or more amino acid residue substitutions or modifications yet retains specific binding affinity to CD3. In some embodiment, at least one of the substitutions or modifications is within one or more of the CDR sequences of the VH region or VL region of the first binding moiety. In some embodiment, at least one of the substitutions or modifications is within one or more of the non-CDR sequences of the VH region or VL region of the first binding moiety.

In some embodiments, the second binding moiety comprises:
one, two or three heavy chain complementarity determining regions (HCDR1, HCDR2 and/or HCDR3) contained within any one of the heavy chain variable (VH) region sequences selected from the group consisting of SEQ ID NOs: 1, 5, 9, 13, 14, 15, 16, 19, 147, 243, 257, 270, 282, 294, 313 and 326; and/or
one, two or three light chain complementarity determining regions (LCDR1, LCDR2 and/or LCDR3) contained within any one of the light chain variable (VL) region sequences selected from the group consisting of SEQ ID NOs: 2, 6, 10, 17, 18, 20, 148, 247, 258, 271, 283, 295, 314 and 327.

In some embodiments, the second binding moiety comprises one, two or three HCDRs contained within SEQ ID NO: 1, and one, two or three LCDRs contained within SEQ ID NO: 2. In some embodiments, the second binding moiety comprises three HCDRs (i.e., HCDR1, HCDR2 and HCDR3) contained within SEQ ID NO: 1, and three LCDRs contained within SEQ ID NO: 2.

In some embodiments, the second binding moiety comprises one, two or three HCDRs contained within SEQ ID NO: 5, and one, two or three LCDRs contained within SEQ ID NO: 6. In some embodiments, the second binding moiety comprises three HCDRs (i.e., HCDR1, HCDR2 and HCDR3) contained within SEQ ID NO: 5, and three LCDRs (i.e., LCDR1, LCDR2 and LCDR3) contained within SEQ ID NO: 6.

In some embodiments, the second binding moiety comprises one, two or three HCDRs contained within SEQ ID NO: 9, and one, two or three LCDRs contained within SEQ ID NO: 10. In some embodiments, the second binding moiety comprises three HCDRs (i.e., HCDR1, HCDR2 and HCDR3) contained within SEQ ID NO: 9, and three LCDRs (i.e., LCDR1, LCDR2 and LCDR3) contained within SEQ ID NO: 10.

In some embodiments, the second binding moiety comprises one, two or three HCDRs contained within SEQ ID NO: 15, and one, two or three LCDRs contained within SEQ ID NO: 18. In some embodiments, the second binding moiety comprises three HCDRs (i.e., HCDR1, HCDR2 and HCDR3) contained within SEQ ID NO: 15, and three LCDRs (i.e., LCDR1, LCDR2 and LCDR3) contained within SEQ ID NO: 18.

In some embodiments, the second binding moiety comprises one, two or three HCDRs contained within SEQ ID NO: 13, and one, two or three LCDRs contained within SEQ ID NO: 18. In some embodiments, the second binding moiety comprises three HCDRs (i.e., HCDR1, HCDR2 and HCDR3) contained within SEQ ID NO: 13, and three LCDRs (i.e., LCDR1, LCDR2 and LCDR3) contained within SEQ ID NO: 18.

In some embodiments, the second binding moiety comprises one, two or three HCDRs contained within SEQ ID NO: 16, and one, two or three LCDRs contained within SEQ ID NO: 17. In some embodiments, the second binding moiety comprises three HCDRs (i.e., HCDR1, HCDR2 and HCDR3) contained within SEQ ID NO: 16, and three LCDRs (i.e., LCDR1, LCDR2 and LCDR3) contained within SEQ ID NO: 17.

In some embodiments, the second binding moiety comprises one, two or three HCDRs contained within SEQ ID NO: 14, and one, two or three LCDRs contained within SEQ ID NO: 18. In some embodiments, the second binding moiety comprises three HCDRs (i.e., HCDR1, HCDR2 and HCDR3) contained within SEQ ID NO: 14, and three LCDRs (i.e., LCDR1, LCDR2 and LCDR3) contained within SEQ ID NO: 18.

In some embodiments, the second binding moiety comprises one, two or three HCDRs contained within SEQ ID NO: 19, and one, two or three LCDRs contained within SEQ ID NO: 20. In some embodiments, the second binding moiety comprises three HCDRs (i.e., HCDR1, HCDR2 and HCDR3) contained within SEQ ID NO: 19, and three LCDRs (i.e., LCDR1, LCDR2 and LCDR3) contained within SEQ ID NO: 20.

In some embodiments, the second binding moiety comprises one, two or three HCDRs contained within SEQ ID NO: 147, and one, two or three LCDRs contained within SEQ ID NO: 148. In some embodiments, the second binding moiety comprises three HCDRs (i.e., HCDR1, HCDR2 and HCDR3) contained within SEQ ID NO: 147, and three LCDRs (i.e., LCDR1, LCDR2 and LCDR3) contained within SEQ ID NO: 148.

In some embodiments, the second binding moiety comprises one, two or three HCDRs contained within SEQ ID NO: 243, and one, two or three LCDRs contained within SEQ ID NO: 247. In some embodiments, the second binding moiety comprises three HCDRs (i.e., HCDR1, HCDR2 and HCDR3) contained within SEQ ID NO: 243, and three LCDRs (i.e., LCDR1, LCDR2 and LCDR3) contained within SEQ ID NO: 247.

In some embodiments, the second binding moiety comprises one, two or three HCDRs contained within SEQ ID NO: 257, and one, two or three LCDRs contained within SEQ ID NO: 258. In some embodiments, the second binding moiety comprises three HCDRs (i.e., HCDR1, HCDR2 and HCDR3) contained within SEQ ID NO: 257, and three LCDRs (i.e., LCDR1, LCDR2 and LCDR3) contained within SEQ ID NO: 258.

In some embodiments, the second binding moiety comprises one, two or three HCDRs contained within SEQ ID NO: 270, and one, two or three LCDRs contained within SEQ ID NO: 271. In some embodiments, the second binding moiety comprises three HCDRs (i.e., HCDR1, HCDR2 and HCDR3) contained within SEQ ID NO: 270, and three LCDRs (i.e., LCDR1, LCDR2 and LCDR3) contained within SEQ ID NO: 271.

In some embodiments, the second binding moiety comprises one, two or three HCDRs contained within SEQ ID NO: 282, and one, two or three LCDRs contained within SEQ ID NO: 283. In some embodiments, the second binding moiety comprises three HCDRs (i.e., HCDR1, HCDR2 and HCDR3) contained within SEQ ID NO: 282, and three LCDRs (i.e., LCDR1, LCDR2 and LCDR3) contained within SEQ ID NO: 283.

In some embodiments, the second binding moiety comprises one, two or three HCDRs contained within SEQ ID NO: 294, and one, two or three LCDRs contained within SEQ ID NO: 295. In some embodiments, the second binding moiety comprises three HCDRs (i.e., HCDR1, HCDR2 and HCDR3) contained within SEQ ID NO: 294, and three LCDRs (i.e., LCDR1, LCDR2 and LCDR3) contained within SEQ ID NO: 295.

In some embodiments, the second binding moiety comprises one, two or three HCDRs contained within SEQ ID NO: 313, and one, two or three LCDRs contained within SEQ ID NO: 314. In some embodiments, the second binding moiety comprises three HCDRs (i.e., HCDR1, HCDR2 and HCDR3) contained within SEQ ID NO: 313, and three LCDRs (i.e., LCDR1, LCDR2 and LCDR3) contained within SEQ ID NO: 314.

In some embodiments, the second binding moiety comprises one, two or three HCDRs contained within SEQ ID NO: 326, and one, two or three LCDRs contained within SEQ ID NO: 327. In some embodiments, the second binding moiety comprises three HCDRs (i.e., HCDR1, HCDR2 and HCDR3) contained within SEQ ID NO: 326, and three LCDRs (i.e., LCDR1, LCDR2 and LCDR3) contained within SEQ ID NO: 327.

In some embodiments, the second binding moiety comprises a VH region having an amino acid sequence as set forth in SEQ ID NOs: 1, 5, 9, 13, 14, 15, 16, 19, 147, 243, 257, 270, 282, 294, 313 and 326, or a homologous sequence thereof having at least 80% sequence identity to SEQ ID NOs: 1, 5, 9, 13, 14, 15, 16, 19, 147, 243, 257, 270, 282, 294, 313 and 326.

In some embodiments, the second binding moiety comprises a VL region having an amino acid sequence as set forth in SEQ ID NOs: 2, 6, 10, 17, 18, 20, 148, 247, 258, 271, 283, 295, 314 or 327, or a homologous sequence thereof having at least 80% sequence identity to SEQ ID NOs: 2, 6, 10, 17, 18, 20, 148, 247, 258, 271, 283, 295, 314 or 327.

In some embodiments, the second binding moiety comprises a VH/VL amino acid sequence pair selected from the group consisting of SEQ ID NOs: 1/2, 5/6, 9/10, 15/18, 13/18, 16/17, 14/18, 19/20, 147/148, 243/247, 257/258, 270/271, 282/283, 294/295, 313/314, and 326/327.

In some embodiments, the antibody or antigen-binding fragment thereof provided herein is linked to one or more conjugate moieties.

In another aspect, the present disclosure provides a chimeric antigen receptor comprising the antibody or antigen-binding fragment thereof provided herein, a transmembrane region and an intracellular signal region.

In another aspect, the present disclosure provides a pharmaceutical composition comprising the antibody or an antigen-binding fragment thereof provided herein, or the chimeric antigen receptor provided herein, and one or more pharmaceutically acceptable carriers.

In another aspect, the present disclosure provides an isolated polynucleotide encoding the antibody or antigen-binding fragment thereof provided herein, and/or the chimeric antigen receptor provided herein.

In another aspect, the present disclosure provides a vector comprising the isolated polynucleotide provided herein.

In another aspect, the present disclosure provides a host expression system comprising the vector provided herein or having the polynucleotide provided herein integrated into genome thereof.

In another aspect, the present disclosure provides a virus comprising the vector provided herein.

In another aspect, the present disclosure provides a kit comprising the antibody or antigen-binding fragment thereof and/or the chimeric antigen receptor and/or the pharmaceutical composition provided herein, and a second therapeutic agent.

In another aspect, the present disclosure provides a method of expressing the antibody or antigen-binding fragment thereof or the chimeric antigen receptor provided herein, comprising culturing the host expression system provided herein under the condition at which the antibody or antigen-binding fragment thereof or the chimeric antigen receptor is expressed.

In another aspect, the present disclosure provides a method of treating, preventing or alleviating a disease, disorder or condition in a subject comprising administering to the subject a therapeutically effective amount of the antibody or antigen-binding fragment thereof and/or the chimeric antigen receptor and/or the pharmaceutical composition provided herein. In some embodiments, the disease, disorder or condition is an immune disease, an autoimmune disease (e.g., autoimmune encephalomyelitis, systemic lupus erythematosus, etc.), an inflammatory disease, a cancer or a neurological disease. In some embodiments, the cancer is a solid tumor or hematologic tumor.

In some embodiments, the administration is through a parenteral route comprising subcutaneous, intraperitoneal, intravenous, intramuscular, or intradermal injection; or a non-parenteral route comprising transdermal, oral, intranasal, intraocular, sublingual, rectal, or topical.

In some embodiments, the method further includes administering to the subject in need thereof an additional therapeutic agent.

In another aspect, the present disclosure provides a method of activating a T cell expressing CD3 or another target other than CD3 in vivo or in vitro, comprising contacting the T cell with the antibody or antigen-binding fragment thereof and/or the chimeric antigen receptor and/or the pharmaceutical composition provided herein.

In another aspect, the present disclosure provides a method of modulating activity of CD3 or another target other than CD3 in a cell expressing CD3 or another target other than CD3, comprising exposing the cell to the antibody or antigen-binding fragment thereof and/or the chimeric antigen receptor and/or the pharmaceutical composition provided herein.

In another aspect, the present disclosure provides a method of promoting in vivo or in vitro processing of a second antigen by a CD3-expressing T cell, comprising contacting the CD3-expressing T cell with the antibody or antigen-binding fragment thereof provided herein, wherein the antibody or antigen-binding fragment thereof is capable of binding to both the CD3-expressing T cell and the second antigen thereby bringing both in close proximity.

In another aspect, the present disclosure provides a method of detecting presence or amount of CD3 or another target other than CD3 in a sample, comprising contacting the sample with the antibody or antigen-binding fragment thereof and/or the chimeric antigen receptor and/or the pharmaceutical composition provided herein, and determining the presence or the amount of CD3 or another target other than CD3 in the sample.

In another aspect, the present disclosure provides a method of diagnosing a disease, disorder or condition related to CD3 or another target other than CD3 in a subject comprises: a) contacting a sample obtained from the subject with the antibody or antigen-binding fragment thereof and/or the chimeric antigen receptor and/or the pharmaceutical composition provided herein; b) determining presence or amount of CD3 or another target other than CD3 in the sample; and c) correlating the presence or the amount of CD3 or another target other than CD3 to existence or status of the disease, disorder or condition in the subject.

In another aspect, the present disclosure provides use the antibody or antigen-binding fragment thereof and/or the chimeric antigen receptor and/or the pharmaceutical composition provided herein in the manufacture of a medicament for treating a disease, disorder or condition related to CD3 or another target other than CD3 in a subject.

In another aspect, the present disclosure provides use the antibody or antigen-binding fragment thereof and/or the chimeric antigen receptor and/or the pharmaceutical composition provided herein in the manufacture of a diagnostic reagent for diagnosing a disease, disorder or condition related to CD3 or another target other than CD3.

In another aspect, the present disclosure provides a kit comprises the antibody or antigen-binding fragment thereof and/or the chimeric antigen receptor and/or the pharmaceutical composition provided herein, useful in detecting CD3 or another target other than CD3, optionally recombinant CD3, CD3 expressed on cell surface, or CD3-expresing cells.

### BRIEF DESCRIPTION OF THE DRAWINGS

FIG. 1 shows T cell binding affinities of ATG-Bis-40E9 and controls.

FIG. 2 shows T-cell dependent cytotoxicity effects of ATG-Bis-40E9 and controls on MM.1S cells.

FIG. 3 shows T-cell dependent cytotoxicity effects of ATG-Bis-40E9 and controls on MOLP-8 cells.

FIG. 4 shows T-cell dependent cytotoxicity effects of ATG-Bis-40E9 and controls on L363 cells.

FIG. 5 shows T-cell dependent cytotoxicity effects of ATG-Bis-40E9 and controls on RPMI8226 cells.

FIG. 6 shows T-cell dependent cytotoxicity effects of ATG-Bis-40E9 and controls on Daudi cells.

FIG. 7 shows IL-2 and IFNγ releases of ATG-Bis-40E9 and controls in T-cell dependent Cytotoxicity.

FIG. 8 shows luciferase assays of ATG-Bis-40E9 and controls on MM.1S cell.

FIG. 9 shows luciferase assays of ATG-Bis-40E9 and controls on MOLP-8 cell.

FIG. 10 shows luciferase assays of ATG-Bis-40E9 and controls on L363 cell.

FIG. 11 shows luciferase assays of ATG-Bis-40E9 and controls on RPMI8226 cell.

FIG. 12 shows luciferase assays of ATG-Bis-40E9 and controls on Daudi cell.

FIG. 13 shows IL-2, IFNγ and IL-6 releases of ATG-Bis-40E9 and controls in PBMC activation.

FIG. 14 shows tumor growth curves of ATG-Bis-40E9 and controls.

FIG. 15 shows T-cell dependent cytotoxicity effects of humanized GPRC5D bispecific antibodies and controls on MM.1S cells.

FIG. 16 shows tumor growth curves of humanized GPRC5D antibodies and controls.

FIG. 17 shows Jurkat cell binding affinities of humanized 2+1 bispecific antibodies and controls.

FIGS. 18A-D show binding affinities of humanized Fab-scFv-Fab bispecific antibody candidates and controls on MM.1S cells (FIG. 18A), MOLP-8 cells (FIG. 18B), L363 cells (FIG. 18C), and RPMI8226 cells (FIG. 18D).

FIGS. 19A-D show T-cell dependent cytotoxicity effects of humanized 2+1 bispecific antibodies and controls on MM.1S cells (FIG. 19A), MOLP-8 cells (FIG. 19B), L363 cells (FIG. 19C), and Daudi cells (FIG. 19D).

FIGS. 20A-E show luciferase assays of humanized Fab-scFv-Fab bispecific antibodies and controls on MM.1S cell (FIG. 20A), MOLP-8 cell (FIG. 20B), L363 cell (FIG. 20C), RPMI8226 cell (FIG. 20D), and Daudi cells (FIG. 20E).

FIG. 21 shows IL-2, IFNγ and IL-6 release of humanized Fab-scFv-Fab bispecific antibody candidates and controls in PBMC activation.

FIG. 22 shows IL-2 and IFNγ releases of humanized Fab-scFv-Fab bispecific antibody candidates and controls in T-cell dependent cytotoxicity.

FIG. 23 shows tumor growth curves of humanized Fab-scFv-Fab bispecific antibodies and controls.

FIG. 24 shows GD2 expression on H82 and SHP77 cells.

FIG. 25 shows H82 cell binding affinities of CD3×GD2 bispecific antibodies with different constructs.

FIG. 26 shows T cell binding affinities of CD3×GD2 bispecific antibodies with different constructs.

FIG. 27 shows T-cell dependent cytotoxicity effects of GD2×CD3 bispecific antibodies on H82 cells.

FIG. 28 shows IL-2 and IFNγ releases of GD2×CD3 bispecific antibodies with different constructs in T-cell dependent cytotoxicity.

FIG. 29 shows luciferase assays of GD2×CD3 bispecific antibodies with different constructs.

FIG. 30 shows diagrams of Fab2-scFv, CrossMab and Fab3 constructs (taking GD2×CD3 bispecific antibody as an example).

FIG. 31 shows Jurkat cell binding affinities of humanized 2+1 bispecific antibodies and controls.

FIG. 32 shows binding affinities of humanized Fab-scFv-Fab bispecific antibody candidates and controls on MM.1S cells.

FIGS. 33A-B show T-cell dependent cytotoxicity effects of humanized 2+1 bispecific antibodies and controls on MM.1S cells (FIG. 33A) and Daudi cells (FIG. 33B).

FIGS. 34A-B show luciferase assays of humanized Fab-scFv-Fab bispecific antibodies and controls on MM.1S cell (FIG. 34A) and Daudi cells (FIG. 34B).

FIG. 35 shows Jurkat cell binding affinities of humanized 2+1 bispecific antibodies and controls.

FIG. 36 shows binding affinities of humanized Fab-scFv-Fab bispecific antibody candidates and controls on MM.1S cells.

FIG. 37 shows T-cell dependent cytotoxicity effect of humanized 2+1 bispecific antibodies and controls on MM.1S cells.

FIG. 38 shows T-cell dependent cytotoxicity effect of humanized 2+1 bispecific antibodies on Daudi cells.

FIG. 39 shows luciferase assays of humanized Fab-scFv-Fab bispecific antibodies and controls on MM.1S cells.

FIG. 40 shows luciferase assays of humanized Fab-scFv-Fab bispecific antibodies and controls on Daudi cells.

FIGS. 41A-D show protein-based ELISA binding analysis of different anti-CD3 antibodies to CD3ε monomer (FIG. 41A), CD3εγ or CD3εδ dimer (FIG. 41B), denatured CD3ε monomer (FIG. 41C), and CD3εδ dimer (FIG. 41D). “40E9-N55S” indicates “40E9-L2H3-N55S.H”.

FIGS. 42A-H show TAA-dependent T cell activation assay results of different CD3 bispecific antibodies and control antibody in the presence of negative target cells.

FIGS. 43A-C show T cell activation assay (43A and B) and T cell dependent cytotoxicity (43C) assay results of different CD3 bispecific antibodies and control antibody in the presence of TAA⁺ cells.

FIG. 44 shows the illustrative constructs of RG6234 and IO-312.

FIGS. 45A-G show binding affinities of several Fab-scFv-Fab bispecific antibody candidates and controls on NALM6 cells (FIGS. 45A and 45B), Raji cells (FIG. 45C), Primary B cells (FIG. 45D), DoHH2 cells (FIG. 45E), SU-DHL-1 cells (FIG. 45F) and Jurkat T cells (FIG. 45G).

FIGS. 46A-C show luciferase assays of several Fab-scFv-Fab bispecific antibodies and controls on NALM6 cells (FIG. 46A), DoHH2 cells (FIG. 46B) and SU-DHL-1 cells (FIG. 46C).

FIGS. 47A-D show T-cell dependent cytotoxicity effects of several Fab-scFv-Fab bispecific antibodies and controls on NALM6 cells (FIG. 47A and FIG. 47B), DoHH2 cells (FIG. 47C), and SU-DHL-1 cells (FIG. 47D).

FIGS. 48A-D show in vivo efficacies of several Fab-scFv-Fab bispecific antibodies and controls in a myelin oligodendrocyte of glycoprotein-induced experimental autoimmune encephalomyelitis (MOG-EAE) model, including efficacy in suppressing disease progression (FIG. 48A), reducing anti-MOG production (FIG. 48B), enhancing cytokine release (FIG. 48C), and lowering inflammation cell infiltration (FIG. 48D).

FIGS. 49A-D show in vivo efficacies of several Fab-scFv-Fab bispecific antibodies and controls in a spontaneous MRL/Ipr SLE model, including efficacy in suppressing disease progression (FIGS. 49A-B), decreasing antinuclear antibody (ANA) production (FIG. 49C), and promoting B cell depletion (FIG. 49D).

### DETAILED DESCRIPTION OF THE INVENTION

The following description of the disclosure is merely intended to illustrate various embodiments of the disclosure. As such, the specific modifications discussed are not to be construed as limitations on the scope of the disclosure. It will be apparent to a person skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entireties.

### Definitions

Throughout the present disclosure, the articles “a”, “an”, and “the” are used herein to refer to one or to more than one (i.e., to at least one) of the grammatical object of the article. By way of example, “an antibody” means one antibody or more than one antibody.

The term “antibody” as used herein includes any immunoglobulin, monoclonal antibody, polyclonal antibody, multivalent antibody, bivalent antibody, monovalent antibody, multi-specific antibody, or bispecific antibody that binds to a specific antigen. A native intact antibody comprises two heavy (H) chains and two light (L) chains. Mammalian heavy chains are classified as alpha, delta, epsilon, gamma, and mu, each heavy chain comprises a variable region (VH) and a first, second, third, and optionally fourth constant region (CH1, CH2, CH3, CH4 respectively); mammalian light chains are classified as λ or κ, while each light chain comprises a variable region (VL) and a constant region. The antibody has a “Y” shape, with the stem of the Y comprising the second and third constant regions of two heavy chains bound together via disulfide bonding. Each arm of the Y includes the variable region and first constant region of a single heavy chain bound to the variable and constant regions of a single light chain. The variable regions of the light and heavy chains are responsible for antigen binding. The variable regions in both chains generally contain three highly variable loops called the complementarity determining regions (CDRs) (light chain CDRs including LCDR1, LCDR2, and LCDR3, heavy chain CDRs including HCDR1, HCDR2, HCDR3). The three CDRs are interposed between flanking stretches known as framework regions (FRs) (light chain FRs including LFR1, LFR2, LFR3, and LFR4, heavy chain FRs including HFR1, HFR2, HFR3, and HFR4), which are more highly conserved than the CDRs and form a scaffold to support the highly variable loops. The constant regions of the heavy and light chains are not involved in antigen-binding, but exhibit various effector functions. Antibodies are assigned to classes based on the amino acid sequences of the constant regions of their heavy chains. The five major classes or isotypes of antibodies are IgA, IgD, IgE, IgG, and IgM, which are characterized by the presence of alpha, delta, epsilon, gamma, and mu heavy chains, respectively. Several of the major antibody classes are divided into subclasses such as IgG1 (gamma1 heavy chain), IgG2 (gamma2 heavy chain), IgG3 (gamma3 heavy chain), IgG4 (gamma4 heavy chain), IgAQ1 (alpha1 heavy chain), or IgA2 (alpha2 heavy chain).

In certain embodiments, the antibody provided herein encompasses any antigen-binding fragments thereof. The term “antigen-binding fragment” as used herein refers to an antibody fragment formed from a portion of an antibody comprising one or more (e.g., 1, 2, 3, 4, 5, or 6) CDRs, or any other antibody fragment that binds to an antigen but does not comprise an intact native antibody structure. Examples of antigen-binding fragments include, without limitation, a diabody, a Fab, a Fab′, a F(ab′)₂, a Fd, an Fv fragment, a disulfide stabilized Fv fragment (dsFv), a (dsFv) 2, a bispecific dsFv (dsFv-dsFv′), a disulfide stabilized diabody (ds diabody), a single-chain antibody molecule (scFv), an scFv dimer (bivalent diabody), a bispecific antibody, a multi-specific antibody, a camelized single domain antibody, a nanobody, a domain antibody, or a bivalent domain antibody. An antigen-binding fragment is capable of binding to the same antigen or epitope to which the parent antibody binds.

“Fab” with regard to an antibody refers to that portion of the antibody consisting of a single light chain (both variable and constant regions) bound to the variable region and first constant region of a single heavy chain by a disulfide bond.

“Fab′” refers to a Fab fragment that includes a portion of the hinge region. “F(ab′)₂” refers to a dimer of Fab′.

“Fc” with regard to an antibody (e.g., of IgG, IgA, or IgD isotype) refers to that portion of the antibody consisting of the second and third constant domains of a first heavy chain bound to the second and third constant domains of a second heavy chain via disulfide bonding. Fc with regard to antibody of IgM and IgE isotype further comprises a fourth constant domain. The Fc portion of the antibody is responsible for various effector functions such as antibody-dependent cell-mediated cytotoxicity (ADCC), and complement dependent cytotoxicity (CDC), but does not function in antigen binding.

“Fv” with regard to an antibody refers to the smallest fragment of the antibody to bear the complete antigen binding site. An Fv fragment consists of the variable region of a single light chain bound to the variable region of a single heavy chain directly or via a linker (e.g., a peptide sequence).

“Single-chain Fv antibody” or “scFv” refers to an engineered antibody consisting of a light chain variable region and a heavy chain variable region connected to one another directly or via a linker (e.g., a peptide sequence) (Huston J S et al., Proc Natl Acad Sci USA, 85: 5879 (1988)).

“Single-chain Fv-Fc antibody” or “scFv-Fc” refers to an engineered antibody consisting of a scFv connected to the Fc region of an antibody directly or via a linker (e.g., a peptide sequence).

“Camelized single domain antibody”, “heavy chain antibody”, or “HCAb” refers to an antibody that contains two VH domains and no light chains (Riechmann L. and Muyldermans S., J Immunol Methods. December 10; 231 (1-2): 25-38 (1999); Muyldermans S., J Biotechnol. Jun; 74 (4): 277-302 (2001); WO94/04678; WO94/25591; U.S. Pat. No. 6,005,079). Heavy chain antibodies were originally derived from Camelidae (camels, dromedaries, and llamas). Although devoid of light chains, camelized antibodies have an authentic antigen-binding repertoire (Hamers-Casterman C. et al., Nature. June 3; 363 (6428): 446-8 (1993); Nguyen V K. et al., Immunogenetics. April; 54 (1): 39-47 (2002); Nguyen V K. et al., Immunology. May; 109 (1): 93-101 (2003)). The variable domain of a heavy chain antibody (VHH domain) represents the smallest known antigen-binding unit generated by adaptive immune responses (Koch-Nolte F. et al., FASEB J. November; 21 (13): 3490-8. Epub 2007 Jun. 15 (2007)).

A “nanobody” refers to an antibody fragment that consists of a VHH domain from a heavy chain antibody and two constant domains, CH2 and CH3.

A “diabody” or “dAb” includes small antibody fragments with two antigen-binding sites, wherein the fragments comprise a VH domain connected to a VL domain in the same polypeptide chain (VH-VL or VL-VH) (see, e.g., Holliger P. et al., Proc Natl Acad Sci USA. July 15; 90 (14): 6444-8 (1993); EP404097; WO93/11161). By using a linker that is too short to allow pairing between the two domains on the same chain, the domains are forced to pair with the complementary domains of another chain, thereby creating two antigen-binding sites. The antigen-binding sites may target the same or different antigens (or epitopes). In certain embodiments, a “bispecific ds diabody” is a diabody target two different antigens (or epitopes).

A “domain antibody” refers to an antibody fragment containing only the variable region of a heavy chain or the variable region of a light chain. In certain instances, two or more VH domains are covalently joined with a peptide linker to create a bivalent or multivalent domain antibody. The two VH domains of a bivalent domain antibody may target the same or different antigens.

The term “valent” as used herein refers to the presence of a specified number of antigen binding sites in a given molecule. The term “monovalent” refers to an antibody or an antigen-binding fragment having only one single antigen-binding site; and the term “multivalent” refers to an antibody or antigen-binding fragment having multiple antigen-binding sites. As such, the terms “bivalent”, “tetravalent”, and “hexavalent” denote the presence of two antigen-binding sites, four antigen-binding sites, and six antigen-binding sites, respectively, in an antigen-binding molecule. In some embodiments, the antibody or antigen-binding fragment thereof is bivalent.

As used herein, a “bispecific” antibody refers to an artificial antibody which has fragments derived from two different monoclonal antibodies and is capable of binding to two different epitopes. The two epitopes may present on the same antigen, or they may present on two different antigens.

As used herein, a “multi-specific” antibody refers to an antibody that specifically binds to at least two distinct antigens or at least two distinct epitopes within the same antigen. Multi-specific antibody may bind for example two, three, four, five or more distinct antigens or distinct epitopes within the same antigen.

In certain embodiments, an “scFv dimer” is a bivalent diabody or bispecific scFv (BsFv) comprising VH-VL (linked by a peptide linker) dimerized with another VH-VL moiety such that VH's of one moiety coordinate with the VL's of the other moiety and form two binding sites which can target the same antigens (or epitopes) or different antigens (or epitopes). In other embodiments, an “scFv dimer” is a bispecific diabody comprising VH1-VL2 (linked by a peptide linker) associated with VL1-VH2 (also linked by a peptide linker) such that VH1 and VL1 coordinate and VH2 and VL2 coordinate and each coordinated pair has a different antigen specificity.

A “dsFv” refers to a disulfide-stabilized Fv fragment that the linkage between the variable region of a single light chain and the variable region of a single heavy chain is a disulfide bond. In some embodiments, a “(dsFv) 2” or “(dsFv-dsFv′)” comprises three peptide chains: two VH moieties linked by a peptide linker (e.g., a long flexible linker) and bound to two VL moieties, respectively, via disulfide bridges. In some embodiments, dsFv-dsFv′ is bispecific in which each disulfide paired heavy and light chain has a different antigen specificity.

The term “chimeric” as used herein, means an antibody or antigen-binding fragment, having a portion of heavy and/or light chain derived from one species, and the rest of the heavy and/or light chain derived from a different species. In an illustrative example, a chimeric antibody may comprise a constant region derived from human and a variable region from a non-human animal, such as from mouse. In some embodiments, the non-human animal is a mammal, for example, a mouse, a rat, a rabbit, a goat, a sheep, a guinea pig, or a hamster.

The term “humanized” as used herein means that the antibody or antigen-binding fragment comprises CDRs derived from non-human animals, FR regions derived from human, and when applicable, the constant regions derived from human. The CDRs of humanized antibodies provided in the present disclosure may contain mutation(s) compared to the CDRs of their parent antibodies.

The term “affinity” as used herein refers to the strength of non-covalent interaction between an immunoglobulin molecule (i.e., antibody) or antigen-binding fragment thereof and an antigen.

An antibody or antigen-binding fragment thereof that “specifically binds” or “specific binding” to a target (e.g., an epitope) is a term well understood in the art, and methods to determine such specific binding are also well known in the art. A molecule is said to exhibit “specific binding” if it reacts or associates more frequently, more rapidly, with greater duration and/or with greater affinity with a particular cell or substance than it does with alternative cells or substances. An antibody “specifically binds” to a target if it binds with greater affinity, avidity, more readily, and/or with greater duration than it binds to other substances. For example, an antibody that specifically binds to a CD3 epitope is an antibody that binds this CD3 epitope with greater affinity, avidity, more readily, and/or with greater duration than it binds to other CD3 epitopes or non-CD3 epitopes. It is also understood by reading this definition that, for example, an antibody (or moiety or epitope) that specifically binds to a first target may or may not specifically bind to a second target. As such, “specific binding” or “specifically bind” does not necessarily require (although it can include) exclusive binding. Generally, but not necessarily, reference to binding means specific binding.

The ability to “compete for binding to CD3” as used herein refers to the ability of a first antibody or antigen-binding fragment to inhibit the binding interaction between CD3 and a second anti-CD3 antibody to any detectable degree. In certain embodiments, an antibody or antigen-binding fragment that competes for binding to CD3 inhibits the binding interaction between CD3 and a second anti-CD3 antibody by at least 85%, or at least 90%. In certain embodiments, this inhibition may be greater than 95%, or greater than 99%.

The term “epitope” as used herein refers to the specific group of atoms or amino acids on an antigen to which an antibody binds. Two antibodies may bind the same or a closely related epitope within an antigen if they exhibit competitive binding for the antigen. An epitope can be linear or conformational (i.e., including amino acid residues spaced apart). For example, if an antibody or antigen-binding fragment blocks binding of a reference antibody to the antigen by at least 85%, or at least 90%, or at least 95%, then the antibody or antigen-binding fragment may be considered to bind the same/closely related epitope as the reference antibody.

The term “amino acid” as used herein refers to an organic compound containing amine (—NH₂) and carboxyl (—COOH) functional groups, along with a side chain specific to each amino acid. The names of amino acids are also represented as standard single letter or three-letter codes in the present disclosure, which are summarized as follows.

| Names | Three-letter Code | Single-letter Code |
|---|---|---|
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

A “conservative substitution” with reference to an amino acid sequence refers to replacing an amino acid residue with a different amino acid residue having a side chain with similar physiochemical properties. For example, conservative substitutions can be made among amino acid residues with hydrophobic side chains (e.g., Met, Ala, Val, Leu, and Ile), among amino acid residues with neutral hydrophilic side chains (e.g., Cys, Ser, Thr, Asn and Gln), among amino acid residues with acidic side chains (e.g., Asp, Glu), among amino acid residues with basic side chains (e.g., His, Lys, and Arg), or among amino acid residues with aromatic side chains (e.g., Trp, Tyr, and Phe). As known in the art, conservative substitution usually does not cause significant change in the protein conformational structure, and therefore could retain the biological activity of a protein.

The term “homologous” as used herein refers to a nucleic acid sequence (or its complementary strand) or an amino acid sequence that has sequence identity of at least 60% (e.g., at least 65%, 70%, 75%, 80%, 85%, 88%, 90%, 91%, 92%, 93%, 94%, 95%, 96%, 97%, 98%, or 99%) to another sequence when optimally aligned.

“Percent (%) sequence identity” with respect to amino acid sequence (or nucleic acid sequence) is defined as the percentage of amino acid (or nucleic acid) residues in a candidate sequence that are identical to the amino acid (or nucleic acid) residues in a reference sequence, after aligning the sequences and, if necessary, introducing gaps, to achieve the maximum number of identical amino acids (or nucleic acids). In other words, percent (%) sequence identity of an amino acid sequence (or nucleic acid sequence) can be calculated by dividing the number of amino acid residues (or bases) that are identical relative to the reference sequence to which it is being compared by the total number of the amino acid residues (or bases) in the candidate sequence or in the reference sequence, whichever is shorter. Conservative substitution of the amino acid residues may or may not be considered as identical residues. Alignment for purposes of determining percent amino acid (or nucleic acid) sequence identity can be achieved, for example, using publicly available tools such as BLASTN, BLASTp (available on the website of U.S. National Center for Biotechnology Information (NCBI), see also, Altschul S. F. et al., J. Mol. Biol., 215:403-410 (1990); Stephen F. et al., Nucleic Acids Res., 25:3389-3402 (1997)), ClustalW2 (available on the website of European Bioinformatics Institute, see also, Higgins D. G. et al., Methods in Enzymology, 266:383-402 (1996); Larkin M. A. et al., Bioinformatics (Oxford, England), 23 (21): 2947-8 (2007)), and ALIGN or Megalign (DNASTAR) software. A person skilled in the art may use the default parameters provided by the tool, or may customize the parameters as appropriate for the alignment, such as for example, by selecting a suitable algorithm.

“Effector functions” as used herein refer to biological activities attributable to the binding of Fc region of an antibody to its effectors such as C1 complex and Fc receptor. Exemplary effector functions include: complement dependent cytotoxicity (CDC) mediated by interaction of antibodies and C1q on the C1 complex; antibody-dependent cell-mediated cytotoxicity (ADCC) mediated by binding of Fc region of an antibody to Fc receptor on an effector cell; and phagocytosis. Effector functions can be evaluated using various assays such as Fc receptor binding assay, C1q binding assay, and cell lysis assay.

“Antibody-dependent cell-mediated cytotoxicity” or “ADCC” as used herein refers to a cell-mediated reaction in which effector cells that express Fc receptors (FcRs) recognize bound antibody or antigen-binding fragment on a target cell and subsequently cause lysis of the target cell. “ADCC activity” or “ADCC effect” refers to the ability of the antibody or antigen-binding fragment which is bound on the target cell to elicit an ADCC reaction as described above.

“Complement dependent cytotoxicity” or “CDC” as used herein refers to a mechanism by which antibodies can mediate specific target cell lysis through activation of an organism's complement system. In CDC, the C1q binds the antibody and this binding triggers the complement cascade which leads to the formation of the membrane attack complex (MAC) (C5b to C9) at the surface of the target cell, as a result of the classical pathway complement activation. “CDC activity” or “CDC effect” refers to the ability of the antibody or antigen-binding fragment which is bound on the target cell to elicit a CDC reaction as described above.

“Target cells” as used herein refer to cells to which antibodies comprising an Fc region specifically bind, generally via the protein part that is C-terminal to the Fc region. “Effector cells” are leukocytes which express one or more Fc receptors and perform effector functions. Examples of human leukocytes which mediate ADCC include peripheral blood mononuclear cells (PBMCs), natural killer (NK) cells, monocytes, cytotoxic T cells and neutrophils; with PBMCs and NK cells being preferred. The effector cells may be isolated from a native source thereof, e.g., from blood or PBMCs as is known in the art.

An “isolated” substance has been altered by the hand of man from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide is “isolated” if it has been sufficiently separated from the coexisting materials of its natural state so as to exist in a substantially pure state. An “isolated nucleic acid sequence” refers to the sequence of an isolated nucleic acid molecule. In certain embodiments, an “isolated antibody or an antigen-binding fragment thereof” refers to the antibody or antigen-binding fragments thereof having a purity of at least 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% as determined by electrophoretic methods (such as SDS-PAGE, isoelectric focusing, capillary electrophoresis), or chromatographic methods (such as ion exchange chromatography or reverse phase HPLC).

The term “vector” as used herein refers to a vehicle into which a polynucleotide encoding a protein may be operably inserted so as to bring about the expression of that protein. A vector may be used to transform, transduce, or transfect a host cell so as to bring about expression of the genetic element it carries within the host cell. Examples of vectors include plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Categories of animal viruses used as vectors include retrovirus (including lentivirus), adenovirus, adeno-associated virus, herpesvirus (e.g., herpes simplex virus), poxvirus, baculovirus, papillomavirus, and papovavirus (e.g., SV40). A vector may contain a variety of elements for controlling expression, including promoter sequences, transcription initiation sequences, enhancer sequences, selectable elements, and reporter genes. In addition, the vector may contain an origin of replication. A vector may also include materials to aid in its entry into the cell, including but not limited to a viral particle, a liposome, or a protein coating. A vector can be an expression vector or a cloning vector. The present disclosure provides vectors (e.g., expression vectors) containing the nucleic acid sequence provided herein encoding the antibody or antigen-binding fragment thereof, at least one promoter (e.g., SV40, CMV, EF-1a) operably linked to the nucleic acid sequence, and at least one selection marker. Examples of vectors include, but are not limited to, retrovirus (including lentivirus), adenovirus, adeno-associated virus, herpesvirus (e.g., herpes simplex virus), poxvirus, baculovirus, papillomavirus, papovavirus (e.g., SV40), lambda phage, and M13 phage, plasmid pcDNA3.3, pMD18-T, pOptivec, pCMV, pEGFP, pIRES, pQD-Hyg-GSeu, pALTER, pBAD, pcDNA, pCal, pL, pET, pGEMEX, pGEX, pCI, pEGFT, pSV2, pFUSE, pVITRO, pVIVO, pMAL, pMONO, pSELECT, pUNO, pDUO, Psg5L, pBABE, pWPXL, pBI, p15TV-L, pPro18, pTD, pRS10, pLexA, pACT2.2, pCMV-SCRIPT.RTM., pCDM8, pCDNA1.1/amp, pcDNA3.1, pRc/RSV, PCR 2.1, pEF-1, pFB, pSG5, pXT1, pCDEF3, pSVSPORT, pEF-Bos etc.

The phrase “host cell” as used herein refers to a cell into which an exogenous polynucleotide and/or a vector can be or has been introduced.

The term “subject” includes human and non-human animals. Non-human animals include all vertebrates, e.g., mammals and non-mammals, such as non-human primates, mice, rats, cats, rabbits, sheep, dogs, cows, chickens, amphibians, and reptiles. Except when noted, the terms “patient”, “subject” or “individual” are used herein interchangeably.

The term “anti-tumor activity” means a reduction in tumor cell proliferation, viability, or metastatic activity. For example, anti-tumor activity can be shown by a decline in growth rate of abnormal cells that arises during therapy or tumor size stability or reduction, or longer survival due to therapy as compared to control without therapy. Such activity can be assessed using accepted in vitro or in vivo tumor models, including but not limited to xenograft models, allograft models, mouse mammary tumor virus (MMTV) models, and other known models known in the art to investigate anti-tumor activity.

“Treating” or “treatment” of a disease, disorder or condition as used herein includes preventing or alleviating a disease, disorder or condition, slowing the onset or rate of development of a disease, disorder or condition, reducing the risk of developing a disease, disorder or condition, preventing or delaying the development of symptoms associated with a disease, disorder or condition, reducing or ending symptoms associated with a disease, disorder or condition, generating a complete or partial regression of a disease, disorder or condition, curing a disease, disorder or condition, or some combination thereof.

The term “diagnosis”, “diagnose” or “diagnosing” refers to the identification of a pathological state, disease or condition, such as identification of a CD3 related disease, or refers to identification of a subject with a CD3 related disease who may benefit from a particular treatment regimen. In some embodiments, diagnosis contains the identification of abnormal amount or activity of CD3. In some embodiments, diagnosis refers to the identification of a cancer in a subject.

As used herein, the term “biological sample” or “sample” refers to a biological composition that is obtained or derived from a subject of interest that contains a cellular and/or other molecular entity that is to be characterized and/or identified, for example based on physical, biochemical, chemical and/or physiological characteristics. A biological sample includes, but is not limited to, cells, tissues, organs and/or biological fluids of a subject, obtained by any method known by those of skill in the art. In some embodiments, the biological sample is a fluid sample. In some embodiments, the fluid sample is whole blood, plasma, blood serum, mucus (including nasal drainage and phlegm), peritoneal fluid, pleural fluid, chest fluid, saliva, urine, synovial fluid, cerebrospinal fluid (CSF), thoracentesis fluid, abdominal fluid, ascites or pericardial fluid. In some embodiments, the biological sample is a tissue or cell obtained from stomach, heart, liver, spleen, lung, kidney, skin or blood vessels of the subject.

“CD3” as used herein, refers to the Cluster of Differentiation 3 protein and includes any variants, conformations, isoforms and species homologs of CD3 which are naturally expressed by cells or are expressed by cells transfected with the CD3 gene. For example, CD3 described herein may refer to the Cluster of Differentiation 3 protein derived from any vertebrate source, including mammals such as primates (e.g., humans, monkeys) and rodents (e.g., mice and rats). In mammals, the CD3 molecule is a multi-protein complex of six chains, including: a CD3gamma chain, a CD3delta chain, two CD3epsilon chains, and a homodimer of CD3zeta chains, wherein the CD3 zeta chain is the intracellular tail of CD3 molecule, and the CD3gamma, CD3delta and CD3epsilon chains all contain extracellular domain (ECD) expressed on surface of T cells. Exemplary sequence of human CD3 includes human CD3epsilon protein (NCBI Ref Seq No. NP_000724), human CD3 delta protein (NCBI Ref Seq No. NP_000723), and human CD3gamma protein (NCBI Ref Seq No. NP_000064). Exemplary sequence of non-human CD3 includes Macaca fascicularis (monkey) CD3epsilon protein (NCBI Ref Seq No. NP_001270544), Macaca fascicularis (monkey) CD 3delta protein (NCBI Ref Seq No. NP_001274617), Macaca fascicularis (monkey) CD3gamma protein (NCBI Ref Seq No. NP_001270839); Mus musculus (mouse) CD3epsilon protein (NCBI Ref Seq No. NP_031674), Mus musculus (mouse) CD3delta protein (NCBI Ref Seq No. NP_038515), Mus musculus domesticus (mouse) CD3gamma protein (NCBI Ref Seq No. AAA37400); Rattus norvegicus (Rat) CD3epsilon protein (NCBI Ref Seq No. NP_001101610), Rattus norvegicus (Rat) CD3delta protein (NCBI Ref Seq No. NP_037301), Rattus norvegicus (Rat) CD3gamma protein (NCBI Ref Seq No. NP_001071114). In certain embodiments, CD3 used herein can also be recombinant CD3, for example, including recombinant CD3epsilon protein, recombinant CD3delta protein, and recombinant CD3gamma protein, which may optionally be expressed as a recombinant CD3 complex. The recombinant CD3 complex may be expressed on a cell surface, or alternatively may be expressed as a soluble form which is not associated on a cell surface. In certain embodiments, the CD3 is human CD3. The terms “CD3”, “CD-3”, “CD 3”, “cluster of differentiation 3” may be used interchangeably in the present disclosure.

The term “anti-CD3 antibody” refers to an antibody that specifically binds to CD3 (e.g., human CD3). The term “anti-human CD3 antibody” refers to an antibody that specifically binds to human CD3. In some embodiments, the anti-CD3 antibody provided herein specifically binds to a CD3gamma protein. In some embodiments, the anti-CD3 antibody provided herein specifically binds to a CD3delta protein. In some embodiments, the anti-CD3 antibody provided herein specifically binds to a CD3epsilon protein. In some embodiments, the anti-CD3 antibody provided herein specifically binds to a conformational epitope of CD3 (e.g., human CD3).

The term “CD3gamma” as used herein is intended to encompass any form of CD3gamma, for example, 1) native unprocessed CD3gamma molecule, “full-length” CD3gamma chain or naturally occurring variants of CD3gamma, including, for example, splice variants or allelic variants; 2) any form of CD3gamma that results from processing in the cell; or 3) full length, a fragment (e.g., a truncated form, an extracellular/transmembrane domain) or a modified form (e.g., a mutated form, a glycosylated/PEGylated, a His-tag/immunofluorescence fused form) of CD3gamma subunit generated through recombinant method.

The term “CD3delta” as used herein is intended to encompass any form of CD3delta, for example, 1) native unprocessed CD3delta molecule, “full-length” CD3delta chain or naturally occurring variants of CD3delta, including, for example, splice variants or allelic variants; 2) any form of CD3delta that results from processing in the cell; or 3) full length, a fragment (e.g., a truncated form, an extracellular/transmembrane domain) or a modified form (e.g., a mutated form, a glycosylated/PEGylated, a His-tag/immunofluorescence fused form) of CD3delta subunit generated through recombinant method.

The term “CD3epsilon” as used herein is intended to encompass any form of CD3epsilon, for example, 1) native unprocessed CD3epsilon molecule, “full-length” CD3epsilon chain or naturally occurring variants of CD3epsilon, including, for example, splice variants or allelic variants; 2) any form of CD3epsilon that results from processing in the cell; or 3) full length, a fragment (e.g., a truncated form, an extracellular/transmembrane domain) or a modified form (e.g., a mutated form, a glycosylated/PEGylated, a His-tag/immunofluorescence fused form) of CD3epsilon subunit generated through recombinant method.

In some embodiments, the anti-CD3 antibody provided herein specifically binds to CD3epsilon, but not binding to CD3gamma (or CD3delta) or binding less well to CD3gamma (or CD3delta), e.g., the binding affinity to CD3epsilon is at least 10-fold lower than that to CD3gamma (or CD3delta), or at least 50-fold lower, or at least 100-fold lower, or at least 200-fold lower than that to CD3gamma (or CD3delta). In some embodiments, the anti-CD3 antibody provided herein does not have detectable binding affinity to CD3gamma (or CD3delta). In some embodiments, the binding affinity is determined by FACS assay. In some embodiments, the binding affinity is determined by Mean Fluorescence Intensity (MFI) detected by FACS assay.

A “CD3 related” or “CD3-related” disease, disorder or condition as used herein refers to any disease, disorder or condition caused by, exacerbated by, or otherwise linked to increased or decreased expression or activities of CD3. In some embodiments, the CD3 related disease, disorder or condition is a disorder related to excessive cell proliferation, such as, for example, cancer. In certain embodiments, the CD3 related disease or condition is characterized in expressing or over-expressing of CD3 and/or CD3 related genes.

The term “pharmaceutically acceptable” indicates that the designated carrier, vehicle, diluent, excipient(s), and/or salt is generally chemically and/or physically compatible with the other ingredients comprising the formulation, and physiologically compatible with the recipient thereof.

The term “CD3-expressing cell” as used herein refer to a cell that expresses CD3 on the surface of the cell.

The term “GPRC5D” as used herein, refers to the G-protein coupled receptor family C group 5 member D, includes any variants, conformations, isoforms and species homologs of GPRC5D which are naturally expressed by cells or are expressed by cells transfected with the GPRC5D gene. For example, GPRC5D described herein may refer to the G-protein coupled receptor family C group 5 member D protein derived from any vertebrate source, including mammals such as primates (e.g., humans, monkeys) and rodents (e.g., mice and rats). Exemplary sequence of human GPRC5D protein is, for example as described in GenBank Accession No. BC069341, NCBI Reference Sequence: NP_061124.1 and UniProtKB/Swiss-Prot Accession No. Q9NZD1 (see also Brauner-Osborne, H. et al., 2001, Biochim. Biophys. Acta 1518, 237-248). The term “GPRC5D” as used herein is intended to encompass any form of GPRC5D, for example, 1) native unprocessed GPRC5D molecule, “full-length” GPRC5D chain or naturally occurring variants of GPRC5D, including, for example, splice variants or allelic variants; 2) any form of GPRC5D that results from processing in the cell; or 3) full length, a fragment (e.g., a truncated form, an extracellular/transmembrane domain) or a modified form (e.g., a mutated form, a glycosylated/PEGylated, a His-tag/immunofluorescence fused form) of GPRC5D subunit generated through recombinant method.

The term “GD2” as used herein, refers to disialoganglioside, which is expressed on tumors of neuroectodermal origin, including human neuroblastoma and melanoma, with highly restricted expression on normal tissues.

The term “LILRB4” as used herein, refers to the leukocyte immunoglobulin-like receptor subfamily B member 4, includes any variants, conformations, isoforms and species homologs of LILRB4 which are naturally expressed by cells or are expressed by cells transfected with the LILRB4 gene. For example, LILRB4 described herein may refer to the leukocyte immunoglobulin-like receptor subfamily B member 4 protein derived from any vertebrate source, including mammals such as primates (e.g., humans, monkeys) and rodents (e.g., mice and rats). Exemplary sequence of human LILRB4 protein is, for example as described in UniProtKB Entry No.: Q8NHJ6 or GenBank Accession No. AAB68665.1. The term “LILRB4” as used herein is intended to encompass any form of LILRB4, for example, 1) native unprocessed LILRB4 molecule, “full-length” LILRB4 chain or naturally occurring variants of LILRB4, including, for example, splice variants or allelic variants; 2) any form of LILRB4 that results from processing in the cell; or 3) full length, a fragment (e.g., a truncated form, an extracellular/transmembrane domain) or a modified form (e.g., a mutated form, a glycosylated/PEGylated, a His-tag/immunofluorescence fused form) of LILRB4 subunit generated through recombinant method.

The CDR, VH and VL amino acid sequences of exemplary first binding moieties that bind to CD3, and the exemplary second or third binding moiety are shown in Tables 22A, 22B, 22C, 22D, 22E, 23, 25, 26, 28, 29, 46, 47, 51, 52, 54 and 55 below.

### Bispecific or Multi-specific Antibodies

In one aspect, the present disclosure provides antibodies or antigen-binding fragments thereof, comprising a first binding moiety that binds to a conformational epitope of CD3, and a second binding moiety that binds to a second target other CD3, wherein the first binding moiety and the second binding moiety are independently a single-chain Fv (scFv) or a Fab domain.

For example, the present disclosure provides bispecific or multi-specific antibodies and antigen-binding fragments thereof, which comprise a first binding moiety that binds to CD3 and a second binding moiety that binds to a second target other than CD3, wherein the first binding moiety and the second binding moiety are independently a single-chain Fv (scFv) or a Fab domain. The bispecific or multi-specific antibodies and antigen-binding fragments provided herein are capable of binding to CD3 (e.g., human CD3), particularly a conformational epitope of CD3.

As used herein, the term “conformational epitope” refers to an epitope wherein the primary sequence of the amino acids comprising the epitope is not the sole defining component of the epitope recognized (e.g., an epitope wherein the primary sequence of amino acids is not necessarily recognized by the binding domain). A “linear” epitope, in contrast to a “conformational” epitope, refers an epitope where an amino acid primary sequence comprises the recognized epitope. One of the differences between “conformational” and “linear” epitopes lies in that the binding to the former but not the latter is lost in the presence of denaturing solvents.

Regarding recognition of conformational epitopes, the binding domain recognizes a three-dimensional structure of the antigen (e.g., CD3), preferably a peptide or protein or fragment thereof. For example, when a protein molecule folds to form a three-dimensional structure, certain amino acids and/or the polypeptide backbone forming the conformational epitope become juxtaposed enabling the antibody to recognize the epitope. Methods of determining the conformation of epitopes include, but are not limited to, x-ray crystallography, two-dimensional nuclear magnetic resonance (2D-NMR) spectroscopy and site-directed spin labelling and electron paramagnetic resonance (EPR) spectroscopy.

In some embodiments, the CD3 binding moiety of the bispecific or multi-specific antibodies, or antigen-binding fragments thereof provided herein binds to a conformational epitope of CD3εγ or CD3εδ dimer. In some embodiments, the CD3 binding moiety of the bispecific or multi-specific antibodies, or antigen-binding fragments thereof provided herein does not bind to a CD3ε monomer.

Binding affinity of the antibody or antigen-binding fragment thereof provided herein can be represented by K_{D} value, which represents the ratio of dissociation rate to association rate (k_{off}/kₒₙ) when the binding between the antigen and antigen-binding molecule reaches equilibrium. The antigen-binding affinity (e.g., K_{D}) can be appropriately determined using suitable methods known in the art, including, for example, flow cytometry assay. In some embodiments, binding of the antibody or antigen-binding fragment thereof to the antigen at different concentrations can be determined by flow cytometry, the determined mean fluorescence intensity (MFI) can be firstly plotted against antibody concentration, K_{D} value can then be calculated by fitting the dependence of specific binding fluorescence intensity (Y) and the concentration of antibodies (X) into the one site saturation equation: Y=Bₘₐₓ *X/(K_{D}+X) using Prism version 5 (GraphPad Software, San Diego, CA), wherein Bₘₐₓ refers to the maximum specific binding of the tested antibody to the antigen.

Binding of the antibodies or the antigen-binding fragments thereof provided herein to CD3 or the second target (e.g., GPCR5D, GD2, LILRB4, CD19, CD20, etc.) can also be represented by “half maximal effective concentration” (EC₅₀) value, which refers to the concentration of an antibody where 50% of its maximal binding is observed. The EC₅₀ value can be measured by binding assays known in the art, for example, direct or indirect binding assay such as enzyme-linked immunosorbent assay (ELISA), FACS assay, and other binding assays. In certain embodiments, the antibodies or antigen-binding fragments thereof provided herein are capable of binding to human CD3, cynomolgus CD3 or mouse CD3 (e.g., as measured by FACS assay). In certain embodiments, the antibodies or antigen-binding fragments thereof provided herein are capable of binding to human GPRC5D, cynomolgus GPRC5D or mouse GPRC5D (e.g., as measured by FACS assay). In certain embodiments, the antibodies or antigen-binding fragments thereof provided herein are capable of binding to human GD2, cynomolgus GD2 or mouse GD2 (e.g., as measured by FACS assay). In certain embodiments, the antibodies or antigen-binding fragments thereof provided herein are capable of binding to human LILRB4, cynomolgus LILRB4 or mouse LILRB4 (e.g., as measured by FACS assay). In certain embodiments, the antibodies or antigen-binding fragments thereof provided herein are capable of binding to human CD19, cynomolgus CD19 or mouse CD19 (e.g., as measured by FACS assay). In certain embodiments, the antibodies or antigen-binding fragments thereof provided herein are capable of binding to human CD20, cynomolgus CD20 or mouse CD20 (e.g., as measured by FACS assay).

In certain embodiments, the antibodies or antigen-binding fragments thereof provided herein are with a T cell activation capability. In certain embodiments, the antibodies or antigen-binding fragments thereof provided herein show potent T cell activation on different multiple myeloma cell lines and higher top RLU values compared with benchmark antibodies JNJ-64407564 and RG6234. JNJ-64407564 and RG6234 are the benchmark antibodies with bi-specificity for the human CD3 and GPRC5D antigens, wherein the construct and amino acid sequences of JNJ-64407564 and RG6234 can be obtained from WO2018017786A2 and WO2019154890A1, respectively. In certain embodiments, the antibodies or antigen-binding fragments thereof provided herein show higher T cell activation than Crossmab format and Fab3 format.

The T cell activation capability of bispecific antibodies can be measured by well-known methods in the art, for example, can be measured by Jurkat NFAT-Luciferase activation assay. In certain embodiments, the T cell activation capability is measured by the method as described in Example 1.4, Example 3.5, Example 4.4, Example 5.5, Example 6.5 and Example 10.3 of the present disclosure.

In certain embodiments, the antibodies or antigen-binding fragments thereof provided herein show T-cell dependent cytotoxicity effect (e.g., as measured by FACS assay). In certain embodiments, the T-cell dependent cytotoxicity effect is measured by the method as described in Example 1.3, Example 2.2, Example 3.4, Example 4.3, Example 5.4, Example 6.4, Example 9.1 and Example 10.4 of the present disclosure.

In certain embodiments, the antibodies or antigen-binding fragments thereof provided herein are with a PBMC activation capability. In certain embodiments, the antibodies or antigen-binding fragments thereof provided herein show potent biosafety in PBMC activation compared with benchmark antibodies JNJ-64407564 and RG6234. The PBMC activation capability of bispecific antibodies can be determined by well-known methods in the art, for example, can be determined by ELISA assay, e.g., measuring IL-2, IFNγ and/or IL-6 release level. In certain embodiments, the PBMC activation capability is measured by the method as described in Example 1.5, Example 3.6, and Example 9.1 of the present disclosure.

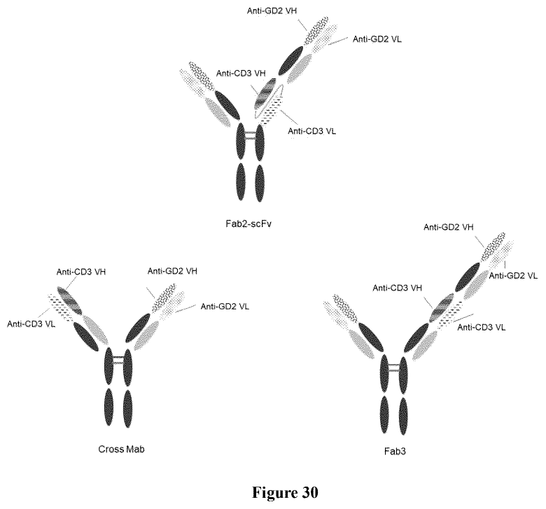

In some embodiments, the antibodies or antigen-binding fragments of the present disclosure are provided in a Fab-scFv-Fab format, which is also referred as “2+1 bispecific”, “chimeric 2+1 bispecific”, “humanized 2+1 bispecific”, “Fab2-scFv” or “Antengager” format in the present disclosure. An antibody in the Fab-scFv-Fab format comprises two Fab domains targeting one or two targets (e.g., GPRC5D, GD2, LILRB4, CD19 or CD20), and one scFv domain target another target (e.g., CD3). An illustrative example of the Fab-scFv-Fab format is shown in FIG. 30 of the present disclosure.

### Illustrative Bispecific or Multi-specific Antibody

First binding moiety

In one aspect, the present disclosure provides antibodies or antigen-binding fragments thereof, comprising a first binding moiety that binds to a conformational epitope of CD3, and a second binding moiety that binds to a second target other than CD3, wherein the first binding moiety and the second binding moiety are independently a single-chain Fv (scFv) or a Fab domain.

In certain embodiments, the present disclosure provides antibodies or antigen-binding fragments thereof, comprising:
i. a first binding moiety that binds to CD3, which comprises one, two or three heavy chain complementarity determining regions (HCDR1, HCDR2 and/or HCDR3) contained within SEQ ID NOs: 3, 7, 11, 21, 22, 23, 26, 27, 28, 29, 30, 80, 82, 83, 84, 85, 86, 119, 120, 122, 124, 125, 126, 128, 131, 134, 136, 138, 139, 145, 195, 196, 197, 200, 201, 202, 203, 204, 205, 207, 208, 209, 210, 211, 221, 222, 223, 224, 225, 226, 227, 228, 229, 230, 231, 232 or 315; and/or one, two or three light chain complementarity determining regions (LCDR1, LCDR2 and/or LCDR3) contained within SEQ ID NOs: 4, 8, 12, 24, 25, 81, 87, 88, 89, 90, 91, 92, 93, 94, 95, 141, 146, 198, 199, 206, 212, 213, 214, 215, 216, 217, 218, 219, 220, 233, 234 or 316;
ii. a second binding moiety that binds to a second target other than CD3,
   wherein the first binding moiety and the second binding moiety are independently a single-chain Fv (scFv) or a Fab domain.

A person skilled in the art can define or identify CDR boundaries of a VH or VL region by well-known methods in the art as long as the amino acid sequence of the VH or VL region is known. For example, CDR boundaries for an antibody or antigen-binding fragment thereof may be defined or identified by the conventions of Kabat, IMGT, Chothia, or Al-Lazikani (Al-Lazikani, B., Chothia, C., Lesk, A. M., J. Mol. Biol., 273 (4), 927 (1997); Chothia, C. et al., J Mol Biol. December 5;186 (3): 651-63 (1985); Chothia, C. and Lesk, A. M., J. Mol. Biol., 196,901 (1987); Chothia, C. et al., Nature. December 21-28;342 (6252): 877-83 (1989); Kabat E. A. et al., Sequences of Proteins of immunological Interest, 5th Ed. Public Health Service, National Institutes of Health, Bethesda, Md. (1991); Marie-Paule Lefranc et al., Developmental and Comparative Immunology, 27:55-77 (2003); Marie-Paule Lefranc et al., Immunome Research, 1 (3), (2005); Marie-Paule Lefranc, Molecular Biology of B cells (second edition), chapter 26, 481-514, (2015)). In some embodiments, the CDR boundaries of the antibodies or antigen-binding fragments thereof provided herein are identified by the convention of Kabat. In some embodiments, the CDR boundaries of the antibodies or antigen-binding fragments thereof provided herein are identified by the convention of IMGT. In some embodiments, the CDR boundaries of the antibodies or antigen-binding fragments thereof provided herein are identified by the convention of Chothia. In some embodiments, the CDR boundaries of the antibodies or antigen-binding fragments thereof provided herein are identified by the convention of Al-Lazikani.

In certain embodiments, the present disclosure provides antibodies or antigen-binding fragments thereof, wherein the first binding moiety binding to CD3 comprises one or more (e.g., 1, 2, 3, 4, 5, or 6) CDR sequences of an anti-CD3 antibody 40E9, 40E9-L2H1, 40E9-L2H3, 40E9-L2H4, 40E9-L3H1, 40E9-L2H3-D99E.H, 40E9-L2H3-Y101F.H, 40E9-L2H3-G106A.H, 40E9-L2H3-Y54G.H, 40E9-L2H3-D105R.H, 40E9-L2H3-N55S.H, 40E9-L2H3-D56G.H, 40E9-L2H3-D105R.H, 40E9-L2H6, 40E9-L2H7, 40E9-L2H9, 40E9-L2H10, 40E9-L2H11, 40E9-L2H14, 40E9-L2H16, 40E9-L5H7 or mCD3-2C11.

Antibody “40E9” as used herein refers to an anti-CD3 antibody or antigen-binding fragment thereof comprising a heavy chain variable region having the sequence of SEQ ID NO: 3, and a light chain variable region having the sequence of SEQ ID NO: 4.

Antibody “40E9-L2H1” as used herein refers to an anti-CD3 antibody or antigen-binding fragment thereof comprising a heavy chain variable region having the sequence of SEQ ID NO: 195, and a light chain variable region having the sequence of SEQ ID NO: 198.

Antibody “40E9-L2H3” as used herein refers to an anti-CD3 antibody or antigen-binding fragment thereof comprising a heavy chain variable region having the sequence of SEQ ID NO: 196, and a light chain variable region having the sequence of SEQ ID NO: 198.

Antibody “40E9-L2H4” as used herein refers to an anti-CD3 antibody or antigen-binding fragment thereof comprising a heavy chain variable region having the sequence of SEQ ID NO: 197, and a light chain variable region having the sequence of SEQ ID NO: 198.

Antibody “40E9-L3H1” as used herein refers to an anti-CD3 antibody or antigen-binding fragment thereof comprising a heavy chain variable region having the sequence of SEQ ID NO: 195, and a light chain variable region having the sequence of SEQ ID NO: 199.

Antibody “40E9-L2H3-D99E.H” as used herein refers to an anti-CD3 antibody or antigen-binding fragment thereof comprising a heavy chain variable region having the sequence of SEQ ID NO: 200, and a light chain variable region having the sequence of SEQ ID NO: 198.

Antibody “40E9-L2H3-Y101F.H” as used herein refers to an anti-CD3 antibody or antigen-binding fragment thereof comprising a heavy chain variable region having the sequence of SEQ ID NO: 201, and a light chain variable region having the sequence of SEQ ID NO: 198.

Antibody “40E9-L2H3-G106A.H” as used herein refers to an anti-CD3 antibody or antigen-binding fragment thereof comprising a heavy chain variable region having the sequence of SEQ ID NO: 202, and a light chain variable region having the sequence of SEQ ID NO: 198.

Antibody “40E9-L2H3-Y54G.H” as used herein refers to an anti-CD3 antibody or antigen-binding fragment thereof comprising a heavy chain variable region having the sequence of SEQ ID NO: 203, and a light chain variable region having the sequence of SEQ ID NO: 198.

Antibody “40E9-L2H3-D105R.H” as used herein refers to an anti-CD3 antibody or antigen-binding fragment thereof comprising a heavy chain variable region having the sequence of SEQ ID NO: 204, and a light chain variable region having the sequence of SEQ ID NO: 198.

Antibody “40E9-L2H3-N55S.H” as used herein refers to an anti-CD3 antibody or antigen-binding fragment thereof comprising a heavy chain variable region having the sequence of SEQ ID NO: 120, and a light chain variable region having the sequence of SEQ ID NO: 198.

Antibody “40E9-L2H3-D56G.H” as used herein refers to an anti-CD3 antibody or antigen-binding fragment thereof comprising a heavy chain variable region having the sequence of SEQ ID NO: 122, and a light chain variable region having the sequence of SEQ ID NO: 198.

Antibody “40E9-L2H3-D105R.H” as used herein refers to an anti-CD3 antibody or antigen-binding fragment thereof comprising a heavy chain variable region having the sequence of SEQ ID NO: 125, and a light chain variable region having the sequence of SEQ ID NO: 198.

Antibody “40E9-L2H6” as used herein refers to an anti-CD3 antibody or antigen-binding fragment thereof comprising a heavy chain variable region having the sequence of SEQ ID NO: 126, and a light chain variable region having the sequence of SEQ ID NO: 198.

Antibody “40E9-L2H7” as used herein refers to an anti-CD3 antibody or antigen-binding fragment thereof comprising a heavy chain variable region having the sequence of SEQ ID NO: 128, and a light chain variable region having the sequence of SEQ ID NO: 198.

Antibody “40E9-L2H9” as used herein refers to an anti-CD3 antibody or antigen-binding fragment thereof comprising a heavy chain variable region having the sequence of SEQ ID NO: 131, and a light chain variable region having the sequence of SEQ ID NO: 198.

Antibody “40E9-L2H10” as used herein refers to an anti-CD3 antibody or antigen-binding fragment thereof comprising a heavy chain variable region having the sequence of SEQ ID NO: 134, and a light chain variable region having the sequence of SEQ ID NO: 198.

Antibody “40E9-L2H11” as used herein refers to an anti-CD3 antibody or antigen-binding fragment thereof comprising a heavy chain variable region having the sequence of SEQ ID NO: 136, and a light chain variable region having the sequence of SEQ ID NO: 198.

Antibody “40E9-L2H14” as used herein refers to an anti-CD3 antibody or antigen-binding fragment thereof comprising a heavy chain variable region having the sequence of SEQ ID NO: 138, and a light chain variable region having the sequence of SEQ ID NO: 198.

Antibody “40E9-L2H16” as used herein refers to an anti-CD3 antibody or antigen-binding fragment thereof comprising a heavy chain variable region having the sequence of SEQ ID NO: 139, and a light chain variable region having the sequence of SEQ ID NO: 198.

Antibody “40E9-L5H7” as used herein refers to an anti-CD3 antibody or antigen-binding fragment thereof comprising a heavy chain variable region having the sequence of SEQ ID NO: 128, and a light chain variable region having the sequence of SEQ ID NO: 141.

Antibody “mCD3-2C11” as used herein refers to an anti-CD3 antibody or antigen-binding fragment thereof comprising a heavy chain variable region having the sequence of SEQ ID NO: 315, and a light chain variable region having the sequence of SEQ ID NO: 316.

In certain embodiments, the present disclosure provides antibodies or antigen-binding fragments thereof, wherein the first binding moiety binding to CD3 comprising one or more (e.g., 1, 2, 3, 4, 5, or 6) CDR sequences of an anti-CD3 antibody 147E11E2, 147-S31R.H, 147-S54R.H, 147-S102Q.H, 147-R100Q.H, 147-S32T.L, 147-S32R.L, 147-K36R.L, 147-N31Y.L, 147-R35K.L, 147-R33A.L, 147-R35S.L, or 147-N37K.L.

Antibody “147E11E2” as used herein refers to an anti-CD3 antibody or antigen-binding fragment thereof comprising a heavy chain variable region having the sequence of SEQ ID NO: 205, and a light chain variable region having the sequence of SEQ ID NO: 206.

Antibody “147-S31R.H” as used herein refers to an anti-CD3 antibody or antigen-binding fragment thereof comprising a heavy chain variable region having the sequence of SEQ ID NO: 208, and a light chain variable region having the sequence of SEQ ID NO: 212.

Antibody “147-S54R.H” as used herein refers to an anti-CD3 antibody or antigen-binding fragment thereof comprising a heavy chain variable region having the sequence of SEQ ID NO: 209, and a light chain variable region having the sequence of SEQ ID NO: 212.

Antibody “147-S102Q.H” as used herein refers to an anti-CD3 antibody or antigen-binding fragment thereof comprising a heavy chain variable region having the sequence of SEQ ID NO: 210, and a light chain variable region having the sequence of SEQ ID NO: 212.

Antibody “147-R100Q.H” as used herein refers to an anti-CD3 antibody or antigen-binding fragment thereof comprising a heavy chain variable region having the sequence of SEQ ID NO: 211, and a light chain variable region having the sequence of SEQ ID NO: 212.

Antibody “147-S32T.L” as used herein refers to an anti-CD3 antibody or antigen-binding fragment thereof comprising a heavy chain variable region having the sequence of SEQ ID NO: 207, and a light chain variable region having the sequence of SEQ ID NO: 213.

Antibody “147-S32R.L” as used herein refers to an anti-CD3 antibody or antigen-binding fragment thereof comprising a heavy chain variable region having the sequence of SEQ ID NO: 207, and a light chain variable region having the sequence of SEQ ID NO: 214.

Antibody “147-K36R.L” as used herein refers to an anti-CD3 antibody or antigen-binding fragment thereof comprising a heavy chain variable region having the sequence of SEQ ID NO: 207, and a light chain variable region having the sequence of SEQ ID NO: 215.

Antibody “147-N31Y.L” as used herein refers to an anti-CD3 antibody or antigen-binding fragment thereof comprising a heavy chain variable region having the sequence of SEQ ID NO: 207, and a light chain variable region having the sequence of SEQ ID NO: 216.

Antibody “147-R35K.L” as used herein refers to an anti-CD3 antibody or antigen-binding fragment thereof comprising a heavy chain variable region having the sequence of SEQ ID NO: 207, and a light chain variable region having the sequence of SEQ ID NO: 217.

Antibody “147-R33A.L” as used herein refers to an anti-CD3 antibody or antigen-binding fragment thereof comprising a heavy chain variable region having the sequence of SEQ ID NO: 207, and a light chain variable region having the sequence of SEQ ID NO: 218.

Antibody “147-R35S.L” as used herein refers to an anti-CD3 antibody or antigen-binding fragment thereof comprising a heavy chain variable region having the sequence of SEQ ID NO: 207, and a light chain variable region having the sequence of SEQ ID NO: 219.

Antibody “147-N37K.L” as used herein refers to an anti-CD3 antibody or antigen-binding fragment thereof comprising a heavy chain variable region having the sequence of SEQ ID NO: 207, and a light chain variable region having the sequence of SEQ ID NO: 220.

In some embodiments, the first binding moiety comprises one, two or three HCDRs contained within SEQ ID NO: 3, and one, two or three LCDRs contained within SEQ ID NO: 4. In some embodiments, the first binding moiety comprises three HCDRs (i.e., HCDR1, HCDR2 and HCDR3) contained within SEQ ID NO: 3, and three LCDRs (i.e., LCDR1, LCDR2 and LCDR3) contained within SEQ ID NO: 4.

In some embodiments, the first binding moiety comprises one, two or three HCDRs contained within SEQ ID NO: 7, and one, two or three LCDRs contained within SEQ ID NO: 8. In some embodiments, the first binding moiety comprises three HCDRs (i.e., HCDR1, HCDR2 and HCDR3) contained within SEQ ID NO: 7, and three LCDRs (i.e., LCDR1, LCDR2 and LCDR3) contained within SEQ ID NO: 8.

In some embodiments, the first binding moiety comprises one, two or three HCDRs contained within SEQ ID NO: 11, and one, two or three LCDRs contained within SEQ ID NO: 12. In some embodiments, the first binding moiety comprises three HCDRs (i.e., HCDR1, HCDR2 and HCDR3) contained within SEQ ID NO: 11, and three LCDRs (i.e., LCDR1, LCDR2 and LCDR3) contained within SEQ ID NO: 12.

In some embodiments, the first binding moiety comprises one, two or three HCDRs contained within SEQ ID NO: 21, and one, two or three LCDRs contained within SEQ ID NO: 24. In some embodiments, the first binding moiety comprises three HCDRs (i.e., HCDR1, HCDR2 and HCDR3) contained within SEQ ID NO: 21, and three LCDRs (i.e., LCDR1, LCDR2 and LCDR3) contained within SEQ ID NO: 24.

In some embodiments, the first binding moiety comprises one, two or three HCDRs contained within SEQ ID NO: 22, and one, two or three LCDRs contained within SEQ ID NO: 24. In some embodiments, the first binding moiety comprises three HCDRs (i.e., HCDR1, HCDR2 and HCDR3) contained within SEQ ID NO: 22, and three LCDRs (i.e., LCDR1, LCDR2 and LCDR3) contained within SEQ ID NO: 24.

In some embodiments, the first binding moiety comprises one, two or three HCDRs contained within SEQ ID NO: 23, and one, two or three LCDRs contained within SEQ ID NO: 24. In some embodiments, the first binding moiety comprises three HCDRs (i.e., HCDR1, HCDR2 and HCDR3) contained within SEQ ID NO: 23, and three LCDRs (i.e., LCDR1, LCDR2 and LCDR3) contained within SEQ ID NO: 24.

In some embodiments, the first binding moiety comprises one, two or three HCDRs contained within SEQ ID NO: 21, and one, two or three LCDRs contained within SEQ ID NO: 25. In some embodiments, the first binding moiety comprises three HCDRs (i.e., HCDR1, HCDR2 and HCDR3) contained within SEQ ID NO: 21, and three LCDRs (i.e., LCDR1, LCDR2 and LCDR3) contained within SEQ ID NO: 25.

In some embodiments, the first binding moiety comprises one, two or three HCDRs contained within SEQ ID NO: 26, and one, two or three LCDRs contained within SEQ ID NO: 24. In some embodiments, the first binding moiety comprises three HCDRs (i.e., HCDR1, HCDR2 and HCDR3) contained within SEQ ID NO: 26, and three LCDRs (i.e., LCDR1, LCDR2 and LCDR3) contained within SEQ ID NO: 24.

In some embodiments, the first binding moiety comprises one, two or three HCDRs contained within SEQ ID NO: 27, and one, two or three LCDRs contained within SEQ ID NO: 24. In some embodiments, the first binding moiety comprises three HCDRs (i.e., HCDR1, HCDR2 and HCDR3) contained within SEQ ID NO: 27, and three LCDRs (i.e., LCDR1, LCDR2 and LCDR3) contained within SEQ ID NO: 24.

In some embodiments, the first binding moiety comprises one, two or three HCDRs contained within SEQ ID NO: 28, and one, two or three LCDRs contained within SEQ ID NO: 24. In some embodiments, the first binding moiety comprises three HCDRs (i.e., HCDR1, HCDR2 and HCDR3) contained within SEQ ID NO: 28, and three LCDRs (i.e., LCDR1, LCDR2 and LCDR3) contained within SEQ ID NO: 24.

In some embodiments, the first binding moiety comprises one, two or three HCDRs contained within SEQ ID NO: 29, and one, two or three LCDRs contained within SEQ ID NO: 24. In some embodiments, the first binding moiety comprises three HCDRs (i.e., HCDR1, HCDR2 and HCDR3) contained within SEQ ID NO: 29, and three LCDRs (i.e., LCDR1, LCDR2 and LCDR3) contained within SEQ ID NO:

24.

In some embodiments, the first binding moiety comprises one, two or three HCDRs contained within SEQ ID NO: 30, and one, two or three LCDRs contained within SEQ ID NO: 24. In some embodiments, the first binding moiety comprises three HCDRs (i.e., HCDR1, HCDR2 and HCDR3) contained within SEQ ID NO: 30, and three LCDRs (i.e., LCDR1, LCDR2 and LCDR3) contained within SEQ ID NO: 24.

In some embodiments, the first binding moiety comprises one, two or three HCDRs contained within SEQ ID NO: 80, and one, two or three LCDRs contained within SEQ ID NO: 81. In some embodiments, the first binding moiety comprises three HCDRs (i.e., HCDR1, HCDR2 and HCDR3) contained within SEQ ID NO: 80, and three LCDRs (i.e., LCDR1, LCDR2 and LCDR3) contained within SEQ ID NO: 81.

In some embodiments, the first binding moiety comprises one, two or three HCDRs contained within SEQ ID NO: 83, and one, two or three LCDRs contained within SEQ ID NO: 87. In some embodiments, the first binding moiety comprises three HCDRs (i.e., HCDR1, HCDR2 and HCDR3) contained within SEQ ID NO: 83, and three LCDRs (i.e., LCDR1, LCDR2 and LCDR3) contained within SEQ ID NO: 87.

In some embodiments, the first binding moiety comprises one, two or three HCDRs contained within SEQ ID NO: 84, and one, two or three LCDRs contained within SEQ ID NO: 87. In some embodiments, the first binding moiety comprises three HCDRs (i.e., HCDR1, HCDR2 and HCDR3) contained within SEQ ID NO: 84, and three LCDRs (i.e., LCDR1, LCDR2 and LCDR3) contained within SEQ ID NO: 87.

In some embodiments, the first binding moiety comprises one, two or three HCDRs contained within SEQ ID NO: 85, and one, two or three LCDRs contained within SEQ ID NO: 87. In some embodiments, the first binding moiety comprises three HCDRs (i.e., HCDR1, HCDR2 and HCDR3) contained within SEQ ID NO: 85, and three LCDRs (i.e., LCDR1, LCDR2 and LCDR3) contained within SEQ ID NO: 87.

In some embodiments, the first binding moiety comprises one, two or three HCDRs contained within SEQ ID NO: 86, and one, two or three LCDRs contained within SEQ ID NO: 87. In some embodiments, the first binding moiety comprises three HCDRs (i.e., HCDR1, HCDR2 and HCDR3) contained within SEQ ID NO: 86, and three LCDRs (i.e., LCDR1, LCDR2 and LCDR3) contained within SEQ ID NO: 87.

In some embodiments, the first binding moiety comprises one, two or three HCDRs contained within SEQ ID NO: 82, and one, two or three LCDRs contained within SEQ ID NO: 88. In some embodiments, the first binding moiety comprises three HCDRs (i.e., HCDR1, HCDR2 and HCDR3) contained within SEQ ID NO: 82, and three LCDRs (i.e., LCDR1, LCDR2 and LCDR3) contained within SEQ ID NO: 88.

In some embodiments, the first binding moiety comprises one, two or three HCDRs contained within SEQ ID NO: 82, and one, two or three LCDRs contained within SEQ ID NO: 89. In some embodiments, the first binding moiety comprises three HCDRs (i.e., HCDR1, HCDR2 and HCDR3) contained within SEQ ID NO: 82, and three LCDRs (i.e., LCDR1, LCDR2 and LCDR3) contained within SEQ ID NO: 89.

In some embodiments, the first binding moiety comprises one, two or three HCDRs contained within SEQ ID NO: 82, and one, two or three LCDRs contained within SEQ ID NO: 90. In some embodiments, the first binding moiety comprises three HCDRs (i.e., HCDR1, HCDR2 and HCDR3) contained within SEQ ID NO: 82, and three LCDRs (i.e., LCDR1, LCDR2 and LCDR3) contained within SEQ ID NO: 90.

In some embodiments, the first binding moiety comprises one, two or three HCDRs contained within SEQ ID NO: 82, and one, two or three LCDRs contained within SEQ ID NO: 91. In some embodiments, the first binding moiety comprises three HCDRs (i.e., HCDR1, HCDR2 and HCDR3) contained within SEQ ID NO: 82, and three LCDRs (i.e., LCDR1, LCDR2 and LCDR3) contained within SEQ ID NO: 91.

In some embodiments, the first binding moiety comprises one, two or three HCDRs contained within SEQ ID NO: 82, and one, two or three LCDRs contained within SEQ ID NO: 92. In some embodiments, the first binding moiety comprises three HCDRs (i.e., HCDR1, HCDR2 and HCDR3) contained within SEQ ID NO: 82, and three LCDRs (i.e., LCDR1, LCDR2 and LCDR3) contained within SEQ ID NO: 92.

In some embodiments, the first binding moiety comprises one, two or three HCDRs contained within SEQ ID NO: 82, and one, two or three LCDRs contained within SEQ ID NO: 93. In some embodiments, the first binding moiety comprises three HCDRs (i.e., HCDR1, HCDR2 and HCDR3) contained within SEQ ID NO: 82, and three LCDRs (i.e., LCDR1, LCDR2 and LCDR3) contained within SEQ ID NO: 93.

In some embodiments, the first binding moiety comprises one, two or three HCDRs contained within SEQ ID NO: 82, and one, two or three LCDRs contained within SEQ ID NO: 94. In some embodiments, the first binding moiety comprises three HCDRs (i.e., HCDR1, HCDR2 and HCDR3) contained within SEQ ID NO: 82, and three LCDRs (i.e., LCDR1, LCDR2 and LCDR3) contained within SEQ ID NO: 94.

In some embodiments, the first binding moiety comprises one, two or three HCDRs contained within SEQ ID NO: 82, and one, two or three LCDRs contained within SEQ ID NO: 95. In some embodiments, the first binding moiety comprises three HCDRs (i.e., HCDR1, HCDR2 and HCDR3) contained within SEQ ID NO: 82, and three LCDRs (i.e., LCDR1, LCDR2 and LCDR3) contained within SEQ ID NO: 95.

In some embodiments, the first binding moiety comprises one, two or three HCDRs contained within SEQ ID NO: 119, and one, two or three LCDRs contained within SEQ ID NO: 198. In some embodiments, the first binding moiety comprises three HCDRs (i.e., HCDR1, HCDR2 and HCDR3) contained within SEQ ID NO: 119, and three LCDRs (i.e., LCDR1, LCDR2 and LCDR3) contained within SEQ ID NO: 198.

In some embodiments, the first binding moiety comprises one, two or three HCDRs contained within SEQ ID NO: 120, and one, two or three LCDRs contained within SEQ ID NO: 198. In some embodiments, the first binding moiety comprises three HCDRs (i.e., HCDR1, HCDR2 and HCDR3) contained within SEQ ID NO: 120, and three LCDRs (i.e., LCDR1, LCDR2 and LCDR3) contained within SEQ ID NO: 198.

In some embodiments, the first binding moiety comprises one, two or three HCDRs contained within SEQ ID NO: 122, and one, two or three LCDRs contained within SEQ ID NO: 198. In some embodiments, the first binding moiety comprises three HCDRs (i.e., HCDR1, HCDR2 and HCDR3) contained within SEQ ID NO: 122, and three LCDRs (i.e., LCDR1, LCDR2 and LCDR3) contained within SEQ ID NO: 198.

In some embodiments, the first binding moiety comprises one, two or three HCDRs contained within SEQ ID NO: 124, and one, two or three LCDRs contained within SEQ ID NO: 198. In some embodiments, the first binding moiety comprises three HCDRs (i.e., HCDR1, HCDR2 and HCDR3) contained within SEQ ID NO: 124, and three LCDRs (i.e., LCDR1, LCDR2 and LCDR3) contained within SEQ ID NO: 198.

In some embodiments, the first binding moiety comprises one, two or three HCDRs contained within SEQ ID NO: 125, and one, two or three LCDRs contained within SEQ ID NO: 198. In some embodiments, the first binding moiety comprises three HCDRs (i.e., HCDR1, HCDR2 and HCDR3) contained within SEQ ID NO: 125, and three LCDRs (i.e., LCDR1, LCDR2 and LCDR3) contained within SEQ ID NO: 198.

In some embodiments, the first binding moiety comprises one, two or three HCDRs contained within SEQ ID NO: 126, and one, two or three LCDRs contained within SEQ ID NO: 198. In some embodiments, the first binding moiety comprises three HCDRs (i.e., HCDR1, HCDR2 and HCDR3) contained within SEQ ID NO: 126, and three LCDRs (i.e., LCDR1, LCDR2 and LCDR3) contained within SEQ ID NO: 198.

In some embodiments, the first binding moiety comprises one, two or three HCDRs contained within SEQ ID NO: 128, and one, two or three LCDRs contained within SEQ ID NO: 198. In some embodiments, the first binding moiety comprises three HCDRs (i.e., HCDR1, HCDR2 and HCDR3) contained within SEQ ID NO: 128, and three LCDRs (i.e., LCDR1, LCDR2 and LCDR3) contained within SEQ ID NO: 198.

In some embodiments, the first binding moiety comprises one, two or three HCDRs contained within SEQ ID NO: 131, and one, two or three LCDRs contained within SEQ ID NO: 198. In some embodiments, the first binding moiety comprises three HCDRs (i.e., HCDR1, HCDR2 and HCDR3) contained within SEQ ID NO: 131, and three LCDRs (i.e., LCDR1, LCDR2 and LCDR3) contained within SEQ ID NO: 198.

In some embodiments, the first binding moiety comprises one, two or three HCDRs contained within SEQ ID NO: 134, and one, two or three LCDRs contained within SEQ ID NO: 198. In some embodiments, the first binding moiety comprises three HCDRs (i.e., HCDR1, HCDR2 and HCDR3) contained within SEQ ID NO: 134, and three LCDRs (i.e., LCDR1, LCDR2 and LCDR3) contained within SEQ ID NO: 198.

In some embodiments, the first binding moiety comprises one, two or three HCDRs contained within SEQ ID NO: 136, and one, two or three LCDRs contained within SEQ ID NO: 198. In some embodiments, the first binding moiety comprises three HCDRs (i.e., HCDR1, HCDR2 and HCDR3) contained within SEQ ID NO: 136, and three LCDRs (i.e., LCDR1, LCDR2 and LCDR3) contained within SEQ ID NO: 198.

In some embodiments, the first binding moiety comprises one, two or three HCDRs contained within SEQ ID NO: 138, and one, two or three LCDRs contained within SEQ ID NO: 198. In some embodiments, the first binding moiety comprises three HCDRs (i.e., HCDR1, HCDR2 and HCDR3) contained within SEQ ID NO: 138, and three LCDRs (i.e., LCDR1, LCDR2 and LCDR3) contained within SEQ ID NO: 198.

In some embodiments, the first binding moiety comprises one, two or three HCDRs contained within SEQ ID NO: 139, and one, two or three LCDRs contained within SEQ ID NO: 198. In some embodiments, the first binding moiety comprises three HCDRs (i.e., HCDR1, HCDR2 and HCDR3) contained within SEQ ID NO: 139, and three LCDRs (i.e., LCDR1, LCDR2 and LCDR3) contained within SEQ ID NO: 198.

In some embodiments, the first binding moiety comprises one, two or three HCDRs contained within SEQ ID NO: 128, and one, two or three LCDRs contained within SEQ ID NO: 141. In some embodiments, the first binding moiety comprises three HCDRs (i.e., HCDR1, HCDR2 and HCDR3) contained within SEQ ID NO: 128, and three LCDRs (i.e., LCDR1, LCDR2 and LCDR3) contained within SEQ ID NO: 141.

In some embodiments, the first binding moiety comprises one, two or three HCDRs contained within SEQ ID NO: 195, and one, two or three LCDRs contained within SEQ ID NO: 198. In some embodiments, the first binding moiety comprises three HCDRs (i.e., HCDR1, HCDR2 and HCDR3) contained within SEQ ID NO: 195, and three LCDRs (i.e., LCDR1, LCDR2 and LCDR3) contained within SEQ ID NO: 198.

In some embodiments, the first binding moiety comprises one, two or three HCDRs contained within SEQ ID NO: 196, and one, two or three LCDRs contained within SEQ ID NO: 198. In some embodiments, the first binding moiety comprises three HCDRs (i.e., HCDR1, HCDR2 and HCDR3) contained within SEQ ID NO: 196, and three LCDRs (i.e., LCDR1, LCDR2 and LCDR3) contained within SEQ ID NO: 198.

In some embodiments, the first binding moiety comprises one, two or three HCDRs contained within SEQ ID NO: 197, and one, two or three LCDRs contained within SEQ ID NO: 198. In some embodiments, the first binding moiety comprises three HCDRs (i.e., HCDR1, HCDR2 and HCDR3) contained within SEQ ID NO: 197, and three LCDRs (i.e., LCDR1, LCDR2 and LCDR3) contained within SEQ ID NO: 198.

In some embodiments, the first binding moiety comprises one, two or three HCDRs contained within SEQ ID NO: 195, and one, two or three LCDRs contained within SEQ ID NO: 199. In some embodiments, the first binding moiety comprises three HCDRs (i.e., HCDR1, HCDR2 and HCDR3) contained within SEQ ID NO: 195, and three LCDRs (i.e., LCDR1, LCDR2 and LCDR3) contained within SEQ ID NO: 199.

In some embodiments, the first binding moiety comprises one, two or three HCDRs contained within SEQ ID NO: 200, and one, two or three LCDRs contained within SEQ ID NO: 198. In some embodiments, the first binding moiety comprises three HCDRs (i.e., HCDR1, HCDR2 and HCDR3) contained within SEQ ID NO: 200, and three LCDRs (i.e., LCDR1, LCDR2 and LCDR3) contained within SEQ ID NO: 198.

In some embodiments, the first binding moiety comprises one, two or three HCDRs contained within SEQ ID NO: 201, and one, two or three LCDRs contained within SEQ ID NO: 198. In some embodiments, the first binding moiety comprises three HCDRs (i.e., HCDR1, HCDR2 and HCDR3) contained within SEQ ID NO: 201, and three LCDRs (i.e., LCDR1, LCDR2 and LCDR3) contained within SEQ ID NO: 198.

In some embodiments, the first binding moiety comprises one, two or three HCDRs contained within SEQ ID NO: 202, and one, two or three LCDRs contained within SEQ ID NO: 198. In some embodiments, the first binding moiety comprises three HCDRs (i.e., HCDR1, HCDR2 and HCDR3) contained within SEQ ID NO: 202, and three LCDRs (i.e., LCDR1, LCDR2 and LCDR3) contained within SEQ ID NO: 198.

In some embodiments, the first binding moiety comprises one, two or three HCDRs contained within SEQ ID NO: 203, and one, two or three LCDRs contained within SEQ ID NO: 198. In some embodiments, the first binding moiety comprises three HCDRs (i.e., HCDR1, HCDR2 and HCDR3) contained within SEQ ID NO: 203, and three LCDRs (i.e., LCDR1, LCDR2 and LCDR3) contained within SEQ ID NO: 198.

In some embodiments, the first binding moiety comprises one, two or three HCDRs contained within SEQ ID NO: 204, and one, two or three LCDRs contained within SEQ ID NO: 198. In some embodiments, the first binding moiety comprises three HCDRs (i.e., HCDR1, HCDR2 and HCDR3) contained within SEQ ID NO: 204, and three LCDRs (i.e., LCDR1, LCDR2 and LCDR3) contained within SEQ ID NO: 198.

In some embodiments, the first binding moiety comprises one, two or three HCDRs contained within SEQ ID NO: 208, and one, two or three LCDRs contained within SEQ ID NO: 212. In some embodiments, the first binding moiety comprises three HCDRs (i.e., HCDR1, HCDR2 and HCDR3) contained within SEQ ID NO: 208, and three LCDRs (i.e., LCDR1, LCDR2 and LCDR3) contained within SEQ ID NO: 212.

In some embodiments, the first binding moiety comprises one, two or three HCDRs contained within SEQ ID NO: 209, and one, two or three LCDRs contained within SEQ ID NO: 212. In some embodiments, the first binding moiety comprises three HCDRs (i.e., HCDR1, HCDR2 and HCDR3) contained within SEQ ID NO: 209, and three LCDRs (i.e., LCDR1, LCDR2 and LCDR3) contained within SEQ ID NO: 212.

In some embodiments, the first binding moiety comprises one, two or three HCDRs contained within SEQ ID NO: 210, and one, two or three LCDRs contained within SEQ ID NO: 212. In some embodiments, the first binding moiety comprises three HCDRs (i.e., HCDR1, HCDR2 and HCDR3) contained within SEQ ID NO: 210, and three LCDRs (i.e., LCDR1, LCDR2 and LCDR3) contained within SEQ ID NO: 212.

In some embodiments, the first binding moiety comprises one, two or three HCDRs contained within SEQ ID NO: 211, and one, two or three LCDRs contained within SEQ ID NO: 212. In some embodiments, the first binding moiety comprises three HCDRs (i.e., HCDR1, HCDR2 and HCDR3) contained within SEQ ID NO: 211, and three LCDRs (i.e., LCDR1, LCDR2 and LCDR3) contained within SEQ ID NO: 212.

In some embodiments, the first binding moiety comprises one, two or three HCDRs contained within SEQ ID NO: 207, and one, two or three LCDRs contained within SEQ ID NO: 213. In some embodiments, the first binding moiety comprises three HCDRs (i.e., HCDR1, HCDR2 and HCDR3) contained within SEQ ID NO: 207, and three LCDRs (i.e., LCDR1, LCDR2 and LCDR3) contained within SEQ ID NO: 213.

In some embodiments, the first binding moiety comprises one, two or three HCDRs contained within SEQ ID NO: 207, and one, two or three LCDRs contained within SEQ ID NO: 214. In some embodiments, the first binding moiety comprises three HCDRs (i.e., HCDR1, HCDR2 and HCDR3) contained within SEQ ID NO: 207, and three LCDRs (i.e., LCDR1, LCDR2 and LCDR3) contained within SEQ ID NO: 214.

In some embodiments, the first binding moiety comprises one, two or three HCDRs contained within SEQ ID NO: 207, and one, two or three LCDRs contained within SEQ ID NO: 215. In some embodiments, the first binding moiety comprises three HCDRs (i.e., HCDR1, HCDR2 and HCDR3) contained within SEQ ID NO: 207, and three LCDRs (i.e., LCDR1, LCDR2 and LCDR3) contained within SEQ ID NO: 215.

In some embodiments, the first binding moiety comprises one, two or three HCDRs contained within SEQ ID NO: 207, and one, two or three LCDRs contained within SEQ ID NO: 216. In some embodiments, the first binding moiety comprises three HCDRs (i.e., HCDR1, HCDR2 and HCDR3) contained within SEQ ID NO: 207, and three LCDRs (i.e., LCDR1, LCDR2 and LCDR3) contained within SEQ ID NO: 216.

In some embodiments, the first binding moiety comprises one, two or three HCDRs contained within SEQ ID NO: 207, and one, two or three LCDRs contained within SEQ ID NO: 217. In some embodiments, the first binding moiety comprises three HCDRs (i.e., HCDR1, HCDR2 and HCDR3) contained within SEQ ID NO: 207, and three LCDRs (i.e., LCDR1, LCDR2 and LCDR3) contained within SEQ ID NO: 217.

In some embodiments, the first binding moiety comprises one, two or three HCDRs contained within SEQ ID NO: 207, and one, two or three LCDRs contained within SEQ ID NO: 218. In some embodiments, the first binding moiety comprises three HCDRs (i.e., HCDR1, HCDR2 and HCDR3) contained within SEQ ID NO: 207, and three LCDRs (i.e., LCDR1, LCDR2 and LCDR3) contained within SEQ ID NO: 218.

In some embodiments, the first binding moiety comprises one, two or three HCDRs contained within SEQ ID NO: 207, and one, two or three LCDRs contained within SEQ ID NO: 219. In some embodiments, the first binding moiety comprises three HCDRs (i.e., HCDR1, HCDR2 and HCDR3) contained within SEQ ID NO: 207, and three LCDRs (i.e., LCDR1, LCDR2 and LCDR3) contained within SEQ ID NO: 219.

In some embodiments, the first binding moiety comprises one, two or three HCDRs contained within SEQ ID NO: 207, and one, two or three LCDRs contained within SEQ ID NO: 220. In some embodiments, the first binding moiety comprises three HCDRs (i.e., HCDR1, HCDR2 and HCDR3) contained within SEQ ID NO: 207, and three LCDRs (i.e., LCDR1, LCDR2 and LCDR3) contained within SEQ ID NO: 220.

In some embodiments, the first binding moiety comprises one, two or three HCDRs contained within SEQ ID NO: 221, and one, two or three LCDRs contained within SEQ ID NO: 233. In some embodiments, the first binding moiety comprises three HCDRs (i.e., HCDR1, HCDR2 and HCDR3) contained within SEQ ID NO: 221, and three LCDRs (i.e., LCDR1, LCDR2 and LCDR3) contained within SEQ ID NO: 233.

In some embodiments, the first binding moiety comprises one, two or three HCDRs contained within SEQ ID NO: 222, and one, two or three LCDRs contained within SEQ ID NO: 233. In some embodiments, the first binding moiety comprises three HCDRs (i.e., HCDR1, HCDR2 and HCDR3) contained within SEQ ID NO: 222, and three LCDRs (i.e., LCDR1, LCDR2 and LCDR3) contained within SEQ ID NO: 233.

In some embodiments, the first binding moiety comprises one, two or three HCDRs contained within SEQ ID NO: 223, and one, two or three LCDRs contained within SEQ ID NO: 233. In some embodiments, the first binding moiety comprises three HCDRs (i.e., HCDR1, HCDR2 and HCDR3) contained within SEQ ID NO: 223, and three LCDRs (i.e., LCDR1, LCDR2 and LCDR3) contained within SEQ ID NO: 233.

In some embodiments, the first binding moiety comprises one, two or three HCDRs contained within SEQ ID NO: 224, and one, two or three LCDRs contained within SEQ ID NO: 233. In some embodiments, the first binding moiety comprises three HCDRs (i.e., HCDR1, HCDR2 and HCDR3) contained within SEQ ID NO: 224, and three LCDRs (i.e., LCDR1, LCDR2 and LCDR3) contained within SEQ ID NO: 233.

In some embodiments, the first binding moiety comprises one, two or three HCDRs contained within SEQ ID NO: 225, and one, two or three LCDRs contained within SEQ ID NO: 233. In some embodiments, the first binding moiety comprises three HCDRs (i.e., HCDR1, HCDR2 and HCDR3) contained within SEQ ID NO: 225, and three LCDRs (i.e., LCDR1, LCDR2 and LCDR3) contained within SEQ ID NO: 233.

In some embodiments, the first binding moiety comprises one, two or three HCDRs contained within SEQ ID NO: 226, and one, two or three LCDRs contained within SEQ ID NO: 233. In some embodiments, the first binding moiety comprises three HCDRs (i.e., HCDR1, HCDR2 and HCDR3) contained within SEQ ID NO: 226, and three LCDRs (i.e., LCDR1, LCDR2 and LCDR3) contained within SEQ ID NO: 233.

In some embodiments, the first binding moiety comprises one, two or three HCDRs contained within SEQ ID NO: 227, and one, two or three LCDRs contained within SEQ ID NO: 233. In some embodiments, the first binding moiety comprises three HCDRs (i.e., HCDR1, HCDR2 and HCDR3) contained within SEQ ID NO: 227, and three LCDRs (i.e., LCDR1, LCDR2 and LCDR3) contained within SEQ ID NO: 233.

In some embodiments, the first binding moiety comprises one, two or three HCDRs contained within SEQ ID NO: 227, and one, two or three LCDRs contained within SEQ ID NO: 234. In some embodiments, the first binding moiety comprises three HCDRs (i.e., HCDR1, HCDR2 and HCDR3) contained within SEQ ID NO: 227, and three LCDRs (i.e., LCDR1, LCDR2 and LCDR3) contained within SEQ ID NO: 234.

In some embodiments, the first binding moiety comprises one, two or three HCDRs contained within SEQ ID NO: 228, and one, two or three LCDRs contained within SEQ ID NO: 233. In some embodiments, the first binding moiety comprises three HCDRs (i.e., HCDR1, HCDR2 and HCDR3) contained within SEQ ID NO: 228, and three LCDRs (i.e., LCDR1, LCDR2 and LCDR3) contained within SEQ ID NO: 233.

In some embodiments, the first binding moiety comprises one, two or three HCDRs contained within SEQ ID NO: 229, and one, two or three LCDRs contained within SEQ ID NO: 233. In some embodiments, the first binding moiety comprises three HCDRs (i.e., HCDR1, HCDR2 and HCDR3) contained within SEQ ID NO: 229, and three LCDRs (i.e., LCDR1, LCDR2 and LCDR3) contained within SEQ ID NO: 233.

In some embodiments, the first binding moiety comprises one, two or three HCDRs contained within SEQ ID NO: 230, and one, two or three LCDRs contained within SEQ ID NO: 233. In some embodiments, the first binding moiety comprises three HCDRs (i.e., HCDR1, HCDR2 and HCDR3) contained within SEQ ID NO: 230, and three LCDRs (i.e., LCDR1, LCDR2 and LCDR3) contained within SEQ ID NO: 233.

In some embodiments, the first binding moiety comprises one, two or three HCDRs contained within SEQ ID NO: 231, and one, two or three LCDRs contained within SEQ ID NO: 233. In some embodiments, the first binding moiety comprises three HCDRs (i.e., HCDR1, HCDR2 and HCDR3) contained within SEQ ID NO: 231, and three LCDRs (i.e., LCDR1, LCDR2 and LCDR3) contained within SEQ ID NO: 233.

In some embodiments, the first binding moiety comprises one, two or three HCDRs contained within SEQ ID NO: 232, and one, two or three LCDRs contained within SEQ ID NO: 233. In some embodiments, the first binding moiety comprises three HCDRs (i.e., HCDR1, HCDR2 and HCDR3) contained within SEQ ID NO: 232, and three LCDRs (i.e., LCDR1, LCDR2 and LCDR3) contained within SEQ ID NO: 233.

In some embodiments, the first binding moiety comprises one, two or three HCDRs contained within SEQ ID NO: 315, and one, two or three LCDRs contained within SEQ ID NO: 316. In some embodiments, the first binding moiety comprises three HCDRs (i.e., HCDR1, HCDR2 and HCDR3) contained within SEQ ID NO: 315, and three LCDRs (i.e., LCDR1, LCDR2 and LCDR3) contained within SEQ ID NO: 316.

In certain embodiments, the first binding moiety of the antibodies or antigen-binding fragments thereof provided herein comprises at least one (e.g., 1, 2, or 3) heavy or light chain CDR comprising an amino acid sequence selected from the group consisting of SEQ ID NOs: 31~55, 96~118, 121, 123, 127, 129, 130, 132, 133, 135, 137, 140, 142, 143, 144, 149, 182~187, 307~312.

In certain embodiments, the first binding moiety of the antibodies or antigen-binding fragments thereof provided herein comprises one or two or three of HCDR1, HCDR2 and HCDR3 comprising an amino acid sequence selected from the group consisting of SEQ ID NOs: 31~40, 44, 45, 46, 50, 51, 52, 96~106, 121, 123, 127, 129, 130, 132, 133, 135, 137, 140, 143, 144, 182, 183, 184, 307, 308 and 309.

In certain embodiments, the first binding moiety of the antibodies or antigen-binding fragments thereof provided herein comprises one or two or three of LCDR1, LCDR2 and LCDR3 comprising an amino acid sequence selected from the group consisting of SEQ ID NOs: 41, 42, 43, 47, 48, 49, 53, 54, 55, 107~118, 142, 149, 185, 186, 187, 310, 311 and 312.

In certain embodiments, the first binding moiety of the antibodies or antigen-binding fragments thereof provided herein comprises a HCDR1 comprising an amino acid sequence selected from the group consisting of SEQ ID NOs: 31, 44, 50, 96, 97, 98, 132, 140, 182 and 307; a HCDR2 comprising an amino acid sequence selected from the group consisting of SEQ ID NOs: 32, 33, 34, 45, 51, 99, 100, 101, 102, 121, 123, 129, 143, 183 and 308; and a HCDR3 comprising an amino acid sequence selected from the group consisting of SEQ ID NOs: 35, 36, 37, 38, 39, 40, 46, 52, 103, 104, 105, 106, 127, 130, 133, 135, 137, 144, 184 and 309.

In certain embodiments, the first binding moiety of the antibodies or antigen-binding fragments thereof provided herein comprises a LCDR1 comprising an amino acid sequence selected from the group consisting of SEQ ID NOs: 41, 47, 53, 107, 108, 109, 110, 111, 112, 113, 114, 115, 116, 185 and 310; a LCDR2 comprising an amino acid sequence selected from the group consisting of SEQ ID NOs: 42, 48, 54, 117, 186 and 311; and a LCDR3 comprising an amino acid sequence selected from the group consisting of SEQ ID NOs: 43, 49, 55, 118, 142, 149, 187 and 312.

In certain embodiments, the first binding moiety of the antibodies or antigen-binding fragments thereof provided herein comprises:
i. a HCDR1 comprising an amino acid sequence as set forth in SEQ ID NO: 31, a HCDR2 comprising an amino acid sequence as set forth in SEQ ID NO: 34, and a HCDR3 comprising an amino acid sequence as set forth in SEQ ID NO: 40;
ii. a HCDR1 comprising an amino acid sequence as set forth in SEQ ID NO: 31, a HCDR2 comprising an amino acid sequence as set forth in SEQ ID NO: 32, and a HCDR3 comprising an amino acid sequence as set forth in SEQ ID NO: 35;
iii. a HCDR1 comprising an amino acid sequence as set forth in SEQ ID NO: 31, a HCDR2 comprising an amino acid sequence as set forth in SEQ ID NO: 32, and a HCDR3 comprising an amino acid sequence as set forth in SEQ ID NO: 36;
iv. a HCDR1 comprising an amino acid sequence as set forth in SEQ ID NO: 31, a HCDR2 comprising an amino acid sequence as set forth in SEQ ID NO: 32, and a HCDR3 comprising an amino acid sequence as set forth in SEQ ID NO: 37;
v. a HCDR1 comprising an amino acid sequence as set forth in SEQ ID NO: 31, a HCDR2 comprising an amino acid sequence as set forth in SEQ ID NO: 32, and a HCDR3 comprising an amino acid sequence as set forth in SEQ ID NO: 38;
vi. a HCDR1 comprising an amino acid sequence as set forth in SEQ ID NO: 31, a HCDR2 comprising an amino acid sequence as set forth in SEQ ID NO: 33, and a HCDR3 comprising an amino acid sequence as set forth in SEQ ID NO: 35;
vii. a HCDR1 comprising an amino acid sequence as set forth in SEQ ID NO: 31, a HCDR2 comprising an amino acid sequence as set forth in SEQ ID NO: 32, and a HCDR3 comprising an amino acid sequence as set forth in SEQ ID NO: 39;
viii. a HCDR1 comprising an amino acid sequence as set forth in SEQ ID NO: 44, a HCDR2 comprising an amino acid sequence as set forth in SEQ ID NO: 45, and a HCDR3 comprising an amino acid sequence as set forth in SEQ ID NO: 46;
ix. a HCDR1 comprising an amino acid sequence as set forth in SEQ ID NO: 50, a HCDR2 comprising an amino acid sequence as set forth in SEQ ID NO: 51, and a HCDR3 comprising an amino acid sequence as set forth in SEQ ID NO: 52;
x. a HCDR1 comprising an amino acid sequence as set forth in SEQ ID NO: 98, a HCDR2 comprising an amino acid sequence as set forth in SEQ ID NO: 102, and a HCDR3 comprising an amino acid sequence as set forth in SEQ ID NO: 106;
xi. a HCDR1 comprising an amino acid sequence as set forth in SEQ ID NO: 96, a HCDR2 comprising an amino acid sequence as set forth in SEQ ID NO: 99, and a HCDR3 comprising an amino acid sequence as set forth in SEQ ID NO: 103;
xii. a HCDR1 comprising an amino acid sequence as set forth in SEQ ID NO: 96, a HCDR2 comprising an amino acid sequence as set forth in SEQ ID NO: 100, and a HCDR3 comprising an amino acid sequence as set forth in SEQ ID NO: 103;
xiii. a HCDR1 comprising an amino acid sequence as set forth in SEQ ID NO: 97, a HCDR2 comprising an amino acid sequence as set forth in SEQ ID NO: 100, and a HCDR3 comprising an amino acid sequence as set forth in SEQ ID NO: 103;
xiv. a HCDR1 comprising an amino acid sequence as set forth in SEQ ID NO: 96, a HCDR2 comprising an amino acid sequence as set forth in SEQ ID NO: 101, and a HCDR3 comprising an amino acid sequence as set forth in SEQ ID NO: 103;
xv. a HCDR1 comprising an amino acid sequence as set forth in SEQ ID NO: 96, a HCDR2 comprising an amino acid sequence as set forth in SEQ ID NO: 100, and a HCDR3 comprising an amino acid sequence as set forth in SEQ ID NO: 104;
xvi. a HCDR1 comprising an amino acid sequence as set forth in SEQ ID NO: 96, a HCDR2 comprising an amino acid sequence as set forth in SEQ ID NO: 100, and a HCDR3 comprising an amino acid sequence as set forth in SEQ ID NO: 105;
xvii. a HCDR1 comprising an amino acid sequence as set forth in SEQ ID NO: 132, a HCDR2 comprising an amino acid sequence as set forth in SEQ ID NO: 143, and a HCDR3 comprising an amino acid sequence as set forth in SEQ ID NO: 144;
xviii. a HCDR1 comprising an amino acid sequence as set forth in SEQ ID NO: 31, a HCDR2 comprising an amino acid sequence as set forth in SEQ ID NO: 121, and a HCDR3 comprising an amino acid sequence as set forth in SEQ ID NO: 35;
xix. a HCDR1 comprising an amino acid sequence as set forth in SEQ ID NO: 31, a HCDR2 comprising an amino acid sequence as set forth in SEQ ID NO: 123, and a HCDR3 comprising an amino acid sequence as set forth in SEQ ID NO: 35;
xx. a HCDR1 comprising an amino acid sequence as set forth in SEQ ID NO: 31, a HCDR2 comprising an amino acid sequence as set forth in SEQ ID NO: 32, and a HCDR3 comprising an amino acid sequence as set forth in SEQ ID NO: 127;
xxi. a HCDR1 comprising an amino acid sequence as set forth in SEQ ID NO: 31, a HCDR2 comprising an amino acid sequence as set forth in SEQ ID NO: 129, and a HCDR3 comprising an amino acid sequence as set forth in SEQ ID NO: 130;
xxii. a HCDR1 comprising an amino acid sequence as set forth in SEQ ID NO: 31, a HCDR2 comprising an amino acid sequence as set forth in SEQ ID NO: 121, and a HCDR3 comprising an amino acid sequence as set forth in SEQ ID NO: 133;
xxiii. a HCDR1 comprising an amino acid sequence as set forth in SEQ ID NO: 31, a HCDR2 comprising an amino acid sequence as set forth in SEQ ID NO: 32, and a HCDR3 comprising an amino acid sequence as set forth in SEQ ID NO: 135;
xxiv. a HCDR1 comprising an amino acid sequence as set forth in SEQ ID NO: 31, a HCDR2 comprising an amino acid sequence as set forth in SEQ ID NO: 32, and a HCDR3 comprising an amino acid sequence as set forth in SEQ ID NO: 137;
xxv. a HCDR1 comprising an amino acid sequence as set forth in SEQ ID NO: 140, a HCDR2 comprising an amino acid sequence as set forth in SEQ ID NO: 32, and a HCDR3 comprising an amino acid sequence as set forth in SEQ ID NO: 35;
xxvi. a HCDR1 comprising an amino acid sequence as set forth in SEQ ID NO: 182, a HCDR2 comprising an amino acid sequence as set forth in SEQ ID NO: 183, and a HCDR3 comprising an amino acid sequence as set forth in SEQ ID NO: 184; or
xxvii. a HCDR1 comprising an amino acid sequence as set forth in SEQ ID NO: 307, a HCDR2 comprising an amino acid sequence as set forth in SEQ ID NO: 308, and a HCDR3 comprising an amino acid sequence as set forth in SEQ ID NO: 309.

In certain embodiments, the first binding moiety of the antibodies or antigen-binding fragments thereof provided herein comprises:
i. a LCDR1 comprising an amino acid sequence as set forth in SEQ ID NO: 41, a LCDR2 comprising an amino acid sequence as set forth in SEQ ID NO: 42, and a LCDR3 comprising an amino acid sequence as set forth in SEQ ID NO: 43;
ii. a LCDR1 comprising an amino acid sequence as set forth in SEQ ID NO: 47, a LCDR2 comprising an amino acid sequence as set forth in SEQ ID NO: 48, and a LCDR3 comprising an amino acid sequence as set forth in SEQ ID NO: 49;
iii. a LCDR1 comprising an amino acid sequence as set forth in SEQ ID NO: 53, a LCDR2 comprising an amino acid sequence as set forth in SEQ ID NO: 54, and a LCDR3 comprising an amino acid sequence as set forth in SEQ ID NO: 55;
iv. a LCDR1 comprising an amino acid sequence as set forth in SEQ ID NO: 116, a LCDR2 comprising an amino acid sequence as set forth in SEQ ID NO: 117, and a LCDR3 comprising an amino acid sequence as set forth in SEQ ID NO: 118;
v. a LCDR1 comprising an amino acid sequence as set forth in SEQ ID NO: 107, a LCDR2 comprising an amino acid sequence as set forth in SEQ ID NO: 117, and a LCDR3 comprising an amino acid sequence as set forth in SEQ ID NO: 118;
vi. a LCDR1 comprising an amino acid sequence as set forth in SEQ ID NO: 108, a LCDR2 comprising an amino acid sequence as set forth in SEQ ID NO: 117, and a LCDR3 comprising an amino acid sequence as set forth in SEQ ID NO: 118;
vii. a LCDR1 comprising an amino acid sequence as set forth in SEQ ID NO: 109, a LCDR2 comprising an amino acid sequence as set forth in SEQ ID NO: 117, and a LCDR3 comprising an amino acid sequence as set forth in SEQ ID NO: 118;
viii. a LCDR1 comprising an amino acid sequence as set forth in SEQ ID NO: 110, a LCDR2 comprising an amino acid sequence as set forth in SEQ ID NO: 117, and a LCDR3 comprising an amino acid sequence as set forth in SEQ ID NO: 118;
ix. a LCDR1 comprising an amino acid sequence as set forth in SEQ ID NO: 111, a LCDR2 comprising an amino acid sequence as set forth in SEQ ID NO: 117, and a LCDR3 comprising an amino acid sequence as set forth in SEQ ID NO: 118;
x. a LCDR1 comprising an amino acid sequence as set forth in SEQ ID NO: 112, a LCDR2 comprising an amino acid sequence as set forth in SEQ ID NO: 117, and a LCDR3 comprising an amino acid sequence as set forth in SEQ ID NO: 118;
xi. a LCDR1 comprising an amino acid sequence as set forth in SEQ ID NO: 113, a LCDR2 comprising an amino acid sequence as set forth in SEQ ID NO: 117, and a LCDR3 comprising an amino acid sequence as set forth in SEQ ID NO: 118;
xii. a LCDR1 comprising an amino acid sequence as set forth in SEQ ID NO: 114, a LCDR2 comprising an amino acid sequence as set forth in SEQ ID NO: 117, and a LCDR3 comprising an amino acid sequence as set forth in SEQ ID NO: 118;
xiii. a LCDR1 comprising an amino acid sequence as set forth in SEQ ID NO: 115, a LCDR2 comprising an amino acid sequence as set forth in SEQ ID NO: 117, and a LCDR3 comprising an amino acid sequence as set forth in SEQ ID NO: 118;
xiv. a LCDR1 comprising an amino acid sequence as set forth in SEQ ID NO: 41, a LCDR2 comprising an amino acid sequence as set forth in SEQ ID NO: 42, and a LCDR3 comprising an amino acid sequence as set forth in SEQ ID NO: 142;
xv. a LCDR1 comprising an amino acid sequence as set forth in SEQ ID NO: 41, a LCDR2 comprising an amino acid sequence as set forth in SEQ ID NO: 42, and a LCDR3 comprising an amino acid sequence as set forth in SEQ ID NO: 149;
xvi. a LCDR1 comprising an amino acid sequence as set forth in SEQ ID NO: 185, a LCDR2 comprising an amino acid sequence as set forth in SEQ ID NO: 186, and a LCDR3 comprising an amino acid sequence as set forth in SEQ ID NO: 187; or
xvii. a LCDR1 comprising an amino acid sequence as set forth in SEQ ID NO: 310, a LCDR2 comprising an amino acid sequence as set forth in SEQ ID NO: 311, and a LCDR3 comprising an amino acid sequence as set forth in SEQ ID NO: 312.

In certain embodiments, the first binding moiety of the antibodies or antigen-binding fragments thereof provided herein comprises:
i. a HCDR1 comprising an amino acid sequence as set forth in SEQ ID NO: 31, a HCDR2 comprising an amino acid sequence as set forth in SEQ ID NO: 34, and a HCDR3 comprising an amino acid sequence as set forth in SEQ ID NO: 40, a LCDR1 comprising an amino acid sequence as set forth in SEQ ID NO: 41, a LCDR2 comprising an amino acid sequence as set forth in SEQ ID NO: 42, and a LCDR3 comprising an amino acid sequence as set forth in SEQ ID NO: 43;
ii. a HCDR1 comprising an amino acid sequence as set forth in SEQ ID NO: 31, a HCDR2 comprising an amino acid sequence as set forth in SEQ ID NO: 32, and a HCDR3 comprising an amino acid sequence as set forth in SEQ ID NO: 35, a LCDR1 comprising an amino acid sequence as set forth in SEQ ID NO: 41, a LCDR2 comprising an amino acid sequence as set forth in SEQ ID NO: 42, and a LCDR3 comprising an amino acid sequence as set forth in SEQ ID NO: 43;
iii. a HCDR1 comprising an amino acid sequence as set forth in SEQ ID NO: 31, a HCDR2 comprising an amino acid sequence as set forth in SEQ ID NO: 32, and a HCDR3 comprising an amino acid sequence as set forth in SEQ ID NO: 36, a LCDR1 comprising an amino acid sequence as set forth in SEQ ID NO: 41, a LCDR2 comprising an amino acid sequence as set forth in SEQ ID NO: 42, and a LCDR3 comprising an amino acid sequence as set forth in SEQ ID NO: 43;
iv. a HCDR1 comprising an amino acid sequence as set forth in SEQ ID NO: 31, a HCDR2 comprising an amino acid sequence as set forth in SEQ ID NO: 32, and a HCDR3 comprising an amino acid sequence as set forth in SEQ ID NO: 37, a LCDR1 comprising an amino acid sequence as set forth in SEQ ID NO: 41, a LCDR2 comprising an amino acid sequence as set forth in SEQ ID NO: 42, and a LCDR3 comprising an amino acid sequence as set forth in SEQ ID NO: 43;
v. a HCDR1 comprising an amino acid sequence as set forth in SEQ ID NO: 31, a HCDR2 comprising an amino acid sequence as set forth in SEQ ID NO: 32, and a HCDR3 comprising an amino acid sequence as set forth in SEQ ID NO: 38, a LCDR1 comprising an amino acid sequence as set forth in SEQ ID NO: 41, a LCDR2 comprising an amino acid sequence as set forth in SEQ ID NO: 42, and a LCDR3 comprising an amino acid sequence as set forth in SEQ ID NO: 43;
vi. a HCDR1 comprising an amino acid sequence as set forth in SEQ ID NO: 31, a HCDR2 comprising an amino acid sequence as set forth in SEQ ID NO: 33, and a HCDR3 comprising an amino acid sequence as set forth in SEQ ID NO: 35, a LCDR1 comprising an amino acid sequence as set forth in SEQ ID NO: 41, a LCDR2 comprising an amino acid sequence as set forth in SEQ ID NO: 42, and a LCDR3 comprising an amino acid sequence as set forth in SEQ ID NO: 43;
vii. a HCDR1 comprising an amino acid sequence as set forth in SEQ ID NO: 31, a HCDR2 comprising an amino acid sequence as set forth in SEQ ID NO: 32, and a HCDR3 comprising an amino acid sequence as set forth in SEQ ID NO: 39, a LCDR1 comprising an amino acid sequence as set forth in SEQ ID NO: 41, a LCDR2 comprising an amino acid sequence as set forth in SEQ ID NO: 42, and a LCDR3 comprising an amino acid sequence as set forth in SEQ ID NO: 43;
viii. a HCDR1 comprising an amino acid sequence as set forth in SEQ ID NO: 44, a HCDR2 comprising an amino acid sequence as set forth in SEQ ID NO: 45, and a HCDR3 comprising an amino acid sequence as set forth in SEQ ID NO: 46, a LCDR1 comprising an amino acid sequence as set forth in SEQ ID NO: 47, a LCDR2 comprising an amino acid sequence as set forth in SEQ ID NO: 48, and a LCDR3 comprising an amino acid sequence as set forth in SEQ ID NO: 49;
ix. a HCDR1 comprising an amino acid sequence as set forth in SEQ ID NO: 50, a HCDR2 comprising an amino acid sequence as set forth in SEQ ID NO: 51, and a HCDR3 comprising an amino acid sequence as set forth in SEQ ID NO: 52, a LCDR1 comprising an amino acid sequence as set forth in SEQ ID NO: 53, a LCDR2 comprising an amino acid sequence as set forth in SEQ ID NO: 54, and a LCDR3 comprising an amino acid sequence as set forth in SEQ ID NO: 55;
x. a HCDR1 comprising an amino acid sequence as set forth in SEQ ID NO: 98, a HCDR2 comprising an amino acid sequence as set forth in SEQ ID NO: 102, and a HCDR3 comprising an amino acid sequence as set forth in SEQ ID NO: 106, a LCDR1 comprising an amino acid sequence as set forth in SEQ ID NO: 116, a LCDR2 comprising an amino acid sequence as set forth in SEQ ID NO: 117, and a LCDR3 comprising an amino acid sequence as set forth in SEQ ID NO: 118;
xi. a HCDR1 comprising an amino acid sequence as set forth in SEQ ID NO: 96, a HCDR2 comprising an amino acid sequence as set forth in SEQ ID NO: 99, and a HCDR3 comprising an amino acid sequence as set forth in SEQ ID NO: 103, a LCDR1 comprising an amino acid sequence as set forth in SEQ ID NO: 107, a LCDR2 comprising an amino acid sequence as set forth in SEQ ID NO: 117, and a LCDR3 comprising an amino acid sequence as set forth in SEQ ID NO: 118;
xii. a HCDR1 comprising an amino acid sequence as set forth in SEQ ID NO: 97, a HCDR2 comprising an amino acid sequence as set forth in SEQ ID NO: 100, and a HCDR3 comprising an amino acid sequence as set forth in SEQ SEQ ID NO: 107, a LCDR2 comprising an amino acid sequence as set forth in SEQ ID NO: 117, and a LCDR3 comprising an amino acid sequence as set forth in SEQ ID NO: 118;
xiii. a HCDR1 comprising an amino acid sequence as set forth in SEQ ID NO: 96, a HCDR2 comprising an amino acid sequence as set forth in SEQ ID NO: 101, and a HCDR3 comprising an amino acid sequence as set forth in SEQ ID NO: 103, a LCDR1 comprising an amino acid sequence as set forth in SEQ ID NO: 107, a LCDR2 comprising an amino acid sequence as set forth in SEQ ID NO: 117, and a LCDR3 comprising an amino acid sequence as set forth in SEQ ID NO: 118;
xiv. a HCDR1 comprising an amino acid sequence as set forth in SEQ ID NO: 96, a HCDR2 comprising an amino acid sequence as set forth in SEQ ID NO: 100, and a HCDR3 comprising an amino acid sequence as set forth in SEQ ID NO: 104, a LCDR1 comprising an amino acid sequence as set forth in SEQ ID NO: 107, a LCDR2 comprising an amino acid sequence as set forth in SEQ ID NO: 117, and a LCDR3 comprising an amino acid sequence as set forth in SEQ ID NO: 118;
xv. a HCDR1 comprising an amino acid sequence as set forth in SEQ ID NO: 96, a HCDR2 comprising an amino acid sequence as set forth in SEQ ID NO: 100, and a HCDR3 comprising an amino acid sequence as set forth in SEQ ID NO: 105, a LCDR1 comprising an amino acid sequence as set forth in SEQ ID NO: 107, a LCDR2 comprising an amino acid sequence as set forth in SEQ ID NO: 117, and a LCDR3 comprising an amino acid sequence as set forth in SEQ ID NO: 118;
xvi. a HCDR1 comprising an amino acid sequence as set forth in SEQ ID NO: 96, a HCDR2 comprising an amino acid sequence as set forth in SEQ ID NO: 100, and a HCDR3 comprising an amino acid sequence as set forth in SEQ ID NO: 103, a LCDR1 comprising an amino acid sequence as set forth in SEQ ID NO: 108, a LCDR2 comprising an amino acid sequence as set forth in SEQ ID NO: 117, and a LCDR3 comprising an amino acid sequence as set forth in SEQ ID NO: 118;
xvii. a HCDR1 comprising an amino acid sequence as set forth in SEQ ID NO: 96, a HCDR2 comprising an amino acid sequence as set forth in SEQ ID NO: 100, and a HCDR3 comprising an amino acid sequence as set forth in SEQ ID NO: 103, a LCDR1 comprising an amino acid sequence as set forth in SEQ ID NO: 109, a LCDR2 comprising an amino acid sequence as set forth in SEQ ID NO: 117, and a LCDR3 comprising an amino acid sequence as set forth in SEQ ID NO: 118;
xviii. a HCDR1 comprising an amino acid sequence as set forth in SEQ ID NO: 96, a HCDR2 comprising an amino acid sequence as set forth in SEQ ID NO: 100, and a HCDR3 comprising an amino acid sequence as set forth in SEQ ID NO: 103, a LCDR1 comprising an amino acid sequence as set forth in SEQ ID NO: 110, a LCDR2 comprising an amino acid sequence as set forth in SEQ ID NO: 117, and a LCDR3 comprising an amino acid sequence as set forth in SEQ ID NO: 118;
xix. a HCDR1 comprising an amino acid sequence as set forth in SEQ ID NO: 96, a HCDR2 comprising an amino acid sequence as set forth in SEQ ID NO: 100, and a HCDR3 comprising an amino acid sequence as set forth in SEQ ID NO: 103, a LCDR1 comprising an amino acid sequence as set forth in SEQ ID NO: 111, a LCDR2 comprising an amino acid sequence as set forth in SEQ ID NO: 117, and a LCDR3 comprising an amino acid sequence as set forth in SEQ ID NO: 118;
XX. a HCDR1 comprising an amino acid sequence as set forth in SEQ ID NO: 96, a HCDR2 comprising an amino acid sequence as set forth in SEQ ID NO: 100, and a HCDR3 comprising an amino acid sequence as set forth in SEQ ID NO: 103, a LCDR1 comprising an amino acid sequence as set forth in SEQ ID NO: 112, a LCDR2 comprising an amino acid sequence as set forth in SEQ ID NO: 117, and a LCDR3 comprising an amino acid sequence as set forth in SEQ ID NO: 118;
xxi. a HCDR1 comprising an amino acid sequence as set forth in SEQ ID NO: 96, a HCDR2 comprising an amino acid sequence as set forth in SEQ ID NO: 100, and a HCDR3 comprising an amino acid sequence as set forth in SEQ ID NO: 103, a LCDR1 comprising an amino acid sequence as set forth in SEQ ID NO: 113, a LCDR2 comprising an amino acid sequence as set forth in SEQ ID NO: 117, and a LCDR3 comprising an amino acid sequence as set forth in SEQ ID NO: 118;
xxii. a HCDR1 comprising an amino acid sequence as set forth in SEQ ID NO: 96, a HCDR2 comprising an amino acid sequence as set forth in SEQ ID NO: 100, and a HCDR3 comprising an amino acid sequence as set forth in SEQ ID NO: 103, a LCDR1 comprising an amino acid sequence as set forth in SEQ ID NO: 114, a LCDR2 comprising an amino acid sequence as set forth in SEQ ID NO: 117, and a LCDR3 comprising an amino acid sequence as set forth in SEQ ID NO: 118;
xxiii. a HCDR1 comprising an amino acid sequence as set forth in SEQ ID NO: 96, a HCDR2 comprising an amino acid sequence as set forth in SEQ ID NO: 100, and a HCDR3 comprising an amino acid sequence as set forth in SEQ ID NO: 103, a LCDR1 comprising an amino acid sequence as set forth in SEQ ID NO: 115, a LCDR2 comprising an amino acid sequence as set forth in SEQ ID NO: 117, and a LCDR3 comprising an amino acid sequence as set forth in SEQ ID NO: 118;
xxiv. a HCDR1 comprising an amino acid sequence as set forth in SEQ ID NO: 31, a HCDR2 comprising an amino acid sequence as set forth in SEQ ID NO: 121, and a HCDR3 comprising an amino acid sequence as set forth in SEQ ID NO: 35, a LCDR1 comprising an amino acid sequence as set forth in SEQ ID NO: 41, a LCDR2 comprising an amino acid sequence as set forth in SEQ ID NO: 42, and a LCDR3 comprising an amino acid sequence as set forth in SEQ ID NO: 43;
xxv. a HCDR1 comprising an amino acid sequence as set forth in SEQ ID NO: 31, a HCDR2 comprising an amino acid sequence as set forth in SEQ ID NO: 123, and a HCDR3 comprising an amino acid sequence as set forth in SEQ ID NO: 35, a LCDR1 comprising an amino acid sequence as set forth in SEQ ID NO: 41, a LCDR2 comprising an amino acid sequence as set forth in SEQ ID NO: 42, and a LCDR3 comprising an amino acid sequence as set forth in SEQ ID NO: 43;
xxvi. a HCDR1 comprising an amino acid sequence as set forth in SEQ ID NO: 31, a HCDR2 comprising an amino acid sequence as set forth in SEQ ID NO: 32, and a HCDR3 comprising an amino acid sequence as set forth in SEQ ID NO: 127, a LCDR1 comprising an amino acid sequence as set forth in SEQ ID NO: 41, a LCDR2 comprising an amino acid sequence as set forth in SEQ ID NO: 42, and a LCDR3 comprising an amino acid sequence as set forth in SEQ ID NO: 43;
xxvii. a HCDR1 comprising an amino acid sequence as set forth in SEQ ID NO: 31, a HCDR2 comprising an amino acid sequence as set forth in SEQ ID NO: 129, and a HCDR3 comprising an amino acid sequence as set forth in SEQ ID NO: 130, a LCDR1 comprising an amino acid sequence as set forth in SEQ ID NO: 41, a LCDR2 comprising an amino acid sequence as set forth in SEQ ID NO: 42, and a LCDR3 comprising an amino acid sequence as set forth in SEQ ID NO: 43;
xxviii. a HCDR1 comprising an amino acid sequence as set forth in SEQ ID NO: 31, a HCDR2 comprising an amino acid sequence as set forth in SEQ ID NO: 121, and a HCDR3 comprising an amino acid sequence as set forth in SEQ ID NO: 133, a LCDR1 comprising an amino acid sequence as set forth in SEQ ID NO: 41, a LCDR2 comprising an amino acid sequence as set forth in SEQ ID NO: 42, and a LCDR3 comprising an amino acid sequence as set forth in SEQ ID NO: 43;
xxix. a HCDR1 comprising an amino acid sequence as set forth in SEQ ID NO: 31, a HCDR2 comprising an amino acid sequence as set forth in SEQ ID NO: 32, and a HCDR3 comprising an amino acid sequence as set forth in SEQ ID NO: 135, a LCDR1 comprising an amino acid sequence as set forth in SEQ ID NO: 41, a LCDR2 comprising an amino acid sequence as set forth in SEQ ID NO: 42, and a LCDR3 comprising an amino acid sequence as set forth in SEQ ID NO: 43;
xxx. a HCDR1 comprising an amino acid sequence as set forth in SEQ ID NO: 31, a HCDR2 comprising an amino acid sequence as set forth in SEQ ID NO: 32, and a HCDR3 comprising an amino acid sequence as set forth in SEQ ID NO: 137, a LCDR1 comprising an amino acid sequence as set forth in SEQ ID NO: 41, a LCDR2 comprising an amino acid sequence as set forth in SEQ ID NO: 42, and a LCDR3 comprising an amino acid sequence as set forth in SEQ ID NO: 43;
xxxi. a HCDR1 comprising an amino acid sequence as set forth in SEQ ID NO: 140, a HCDR2 comprising an amino acid sequence as set forth in SEQ ID NO: 32, and a HCDR3 comprising an amino acid sequence as set forth in SEQ ID NO: 35, a LCDR1 comprising an amino acid sequence as set forth in SEQ ID NO: 41, a LCDR2 comprising an amino acid sequence as set forth in SEQ ID NO: 42, and a LCDR3 comprising an amino acid sequence as set forth in SEQ ID NO: 43; or
xxxii. a HCDR1 comprising an amino acid sequence as set forth in SEQ ID NO: 132, a HCDR2 comprising an amino acid sequence as set forth in SEQ ID NO: 143, and a HCDR3 comprising an amino acid sequence as set forth in SEQ ID NO: 144, a LCDR1 comprising an amino acid sequence as set forth in SEQ ID NO: 41, a LCDR2 comprising an amino acid sequence as set forth in SEQ ID NO: 42, and a LCDR3 comprising an amino acid sequence as set forth in SEQ ID NO: 149;
xxxiii. a HCDR1 comprising an amino acid sequence as set forth in SEQ ID NO: 31, a HCDR2 comprising an amino acid sequence as set forth in SEQ ID NO: 129, and a HCDR3 comprising an amino acid sequence as set forth in SEQ ID NO: 130, a LCDR1 comprising an amino acid sequence as set forth in SEQ ID NO: 41, a LCDR2 comprising an amino acid sequence as set forth in SEQ ID NO: 42, and a LCDR3 comprising an amino acid sequence as set forth in SEQ ID NO: 142; or
xxxiv. a HCDR1 comprising an amino acid sequence as set forth in SEQ ID NO: 307, a HCDR2 comprising an amino acid sequence as set forth in SEQ ID NO: 308, and a HCDR3 comprising an amino acid sequence as set forth in SEQ ID NO: 309, a LCDR1 comprising an amino acid sequence as set forth in SEQ ID NO: 310, a LCDR2 comprising an amino acid sequence as set forth in SEQ ID NO: 311, and a LCDR3 comprising an amino acid sequence as set forth in SEQ ID NO: 312.

In certain embodiments, the first binding moiety of the antibodies or antigen-binding fragments thereof provided herein comprises a VH region comprising one or two or three of HCDR1, HCDR2 and HCDR3 comprising an amino acid sequence selected from the group consisting of SEQ ID NOs: 31~40, 44, 45, 46, 50, 51, 52, 96~106, 121, 123, 127, 129, 130, 132, 133, 135, 137, 140, 143, 144, 182, 183, 184, 307, 308 and 309.

In certain embodiments, the first binding moiety of the antibodies or antigen-binding fragments thereof provided herein comprises a VL region comprising one or two or three of LCDR1, LCDR2 and LCDR3 comprising an amino acid sequence selected from the group consisting of SEQ ID NOs: 41, 42, 43, 47, 48, 49, 53, 54, 55, 107~118, 142, 149, 185, 186, 187, 310, 311 and 312.

The SEQ ID NOs of the heavy chain (denoted as “H”) variable region, light chain (denoted as “L”) variable region, HCDRs and LCDRs of each of the exemplary first binding moieties that bind to CD3 described above are shown in Table 21A and Table 21B below. The amino acid sequences of each CDR of the exemplary first binding moieties that bind to CD3 are shown in Table 22A, Table 22B, Table 22C, Table 22D and Table 22E below. Unless otherwise indicated, the CDR boundaries as described in Table 21A and Table 21B below were defined or identified by the convention of Kabat. The amino acid sequences of each VH and VL of the exemplary first binding moieties that bind to CD3 are shown in Table 23 below.

TABLE 21A
SEQ ID NOs of VH, VL, HCDRs and LCDRs of Exemplary
First Binding Moieties that Bind to CD3

| | | Variable | CDR1 | CDR2 | CDR3 |
|---|---|---|---|---|---|
| | | Region | (SEQ | (SEQ | (SEQ |
| | | (SEQ | ID | ID | ID |
| Antibody# | Chain | ID NO) | NO) | NO) | NO) |
| 40E9 | H | 3 | 31 | 32 | 35 |
| | L | 4 | 41 | 42 | 43 |
| JNJ-64407564 | H | 7 | 44 | 45 | 46 |
| (anti-CD3 moiety) | L | 8 | 47 | 48 | 49 |
| RG6234 (anti-CD3 | H | 11 | 50 | 51 | 52 |
| moiety) | L | 12 | 53 | 54 | 55 |
| 10001 | H | 21 | 31 | 32 | 35 |
| | L | 24 | 41 | 42 | 43 |
| 10002 | H | 22 | 31 | 32 | 35 |
| | L | 24 | 41 | 42 | 43 |
| 10003 | H | 23 | 31 | 32 | 35 |
| | L | 24 | 41 | 42 | 43 |
| 10004 | H | 26 | 31 | 32 | 36 |
| | L | 24 | 41 | 42 | 43 |
| 10005 | H | 27 | 31 | 32 | 37 |
| | L | 24 | 41 | 42 | 43 |
| 10006 | H | 28 | 31 | 32 | 38 |
| | L | 24 | 41 | 42 | 43 |
| 10007 | H | 29 | 31 | 33 | 35 |
| | L | 24 | 41 | 42 | 43 |
| 10008 | H | 30 | 31 | 32 | 39 |
| | L | 24 | 41 | 42 | 43 |
| 10009 | H | 21 | 31 | 32 | 35 |
| | L | 25 | 41 | 42 | 43 |
| 10010 | H | 80 | 96 | 99 | 103 |
| | L | 81 | 107 | 117 | 118 |
| 10011 | H | 83 | 97 | 100 | 103 |
| | L | 87 | 107 | 117 | 118 |
| 10012 | H | 84 | 96 | 101 | 103 |
| | L | 87 | 107 | 117 | 118 |
| 10013 | H | 85 | 96 | 100 | 104 |
| | L | 87 | 107 | 117 | 118 |
| 10014 | H | 86 | 96 | 100 | 105 |
| | L | 87 | 107 | 117 | 118 |
| 10015 | H | 82 | 96 | 100 | 103 |
| | L | 88 | 108 | 117 | 118 |
| 10016 | H | 82 | 96 | 100 | 103 |
| | L | 89 | 109 | 117 | 118 |
| 10017 | H | 82 | 96 | 100 | 103 |
| | L | 90 | 110 | 117 | 118 |
| 10018 | H | 82 | 96 | 100 | 103 |
| | L | 91 | 111 | 117 | 118 |
| 10019 | H | 82 | 96 | 100 | 103 |
| | L | 92 | 112 | 117 | 118 |
| 10020 | H | 82 | 96 | 100 | 103 |
| | L | 93 | 113 | 117 | 118 |
| 10021 | H | 82 | 96 | 100 | 103 |
| | L | 94 | 114 | 117 | 118 |
| 10022 | H | 82 | 96 | 100 | 103 |
| | L | 95 | 115 | 117 | 118 |
| 10023 | H | 221 | 31 | 32 | 35 |
| | L | 233 | 41 | 42 | 43 |
| 10024 | H | 222 | 31 | 121 | 35 |
| | L | 233 | 41 | 42 | 43 |
| 10025 | H | 223 | 31 | 123 | 35 |
| | L | 233 | 41 | 42 | 43 |
| 10026 | H | 224 | 31 | 32 | 37 |
| | L | 233 | 41 | 42 | 43 |
| 10027 | H | 225 | 31 | 32 | 39 |
| | L | 233 | 41 | 42 | 43 |
| 10028 | H | 226 | 31 | 32 | 127 |
| | L | 233 | 41 | 42 | 43 |
| 10029 | H | 227 | 31 | 129 | 130 |
| | L | 233 | 41 | 42 | 43 |
| 10030 | H | 228 | 31 | 121 | 133 |
| | L | 233 | 41 | 42 | 43 |
| 10031 | H | 229 | 31 | 32 | 135 |
| | L | 233 | 41 | 42 | 43 |
| 10032 | H | 230 | 31 | 32 | 137 |
| | L | 233 | 41 | 42 | 43 |
| 10033 | H | 231 | 31 | 32 | 36 |
| | L | 233 | 41 | 42 | 43 |
| 10034 | H | 232 | 140 | 32 | 35 |
| | L | 233 | 41 | 42 | 43 |
| 10035 | H | 227 | 31 | 129 | 130 |
| | L | 234 | 41 | 42 | 142 |

TABLE 21B
SEQ ID NOs of VH, VL, HCDRs and LCDRs of Exemplary
First Binding Moieties that Bind to CD3

| | | Variable | CDR1 | CDR2 | CDR3 |
|---|---|---|---|---|---|
| | | Region | (SEQ | (SEQ | (SEQ |
| | | (SEQ | ID | ID | ID |
| Antibody | Chain | ID NO) | NO) | NO) | NO) |
| 40E9-L2H1 | H | 195 | 31 | 32 | 35 |
| | L | 198 | 41 | 42 | 43 |
| 40E9-L2H3 | H | 196 | 31 | 32 | 35 |
| | L | 198 | 41 | 42 | 43 |
| 40E9-L2H4 | H | 197 | 31 | 32 | 35 |
| | L | 198 | 41 | 42 | 43 |
| 40E9-L2H3-D99E.H | H | 200 | 31 | 32 | 36 |
| | L | 198 | 41 | 42 | 43 |
| 40E9-L2H3-Y101F.H | H | 201 | 31 | 32 | 37 |
| | L | 198 | 41 | 42 | 43 |
| 40E9-L2H3-G106A.H | H | 202 | 31 | 32 | 38 |
| | L | 198 | 41 | 42 | 43 |
| 40E9-L2H3-Y54G.H | H | 203 | 31 | 33 | 35 |
| | L | 198 | 41 | 42 | 43 |
| 40E9-L2H3-D105R.H | H | 204 | 31 | 32 | 39 |
| | L | 198 | 41 | 42 | 43 |
| 40E9-L3H1 | H | 195 | 31 | 32 | 35 |
| | L | 199 | 41 | 42 | 43 |
| 147E11E2 | H | 80 | 96 | 99 | 103 |
| | L | 81 | 107 | 117 | 118 |
| 147-S31R.H | H | 208 | 97 | 100 | 103 |
| | L | 212 | 107 | 117 | 118 |
| 147-S54R.H | H | 209 | 96 | 101 | 103 |
| | L | 212 | 107 | 117 | 118 |
| 147-S102Q.H | H | 210 | 96 | 100 | 104 |
| | L | 212 | 107 | 117 | 118 |
| 147-R100Q.H | H | 211 | 96 | 100 | 105 |
| | L | 212 | 107 | 117 | 118 |
| 147-S32T.L | H | 207 | 96 | 100 | 103 |
| | L | 213 | 108 | 117 | 118 |
| 147-S32R.L | H | 207 | 96 | 100 | 103 |
| | L | 214 | 109 | 117 | 118 |
| 147-K36R.L | H | 207 | 96 | 100 | 103 |
| | L | 215 | 110 | 117 | 118 |
| 147-N31Y.L | H | 207 | 96 | 100 | 103 |
| | L | 216 | 111 | 117 | 118 |
| 147-R35K.L | H | 207 | 96 | 100 | 103 |
| | L | 217 | 112 | 117 | 118 |
| 147-R33A.L | H | 207 | 96 | 100 | 103 |
| | L | 218 | 113 | 117 | 118 |
| 147-R35S.L | H | 207 | 96 | 100 | 103 |
| | L | 219 | 114 | 117 | 118 |
| 147-N37K.L | H | 207 | 96 | 100 | 103 |
| | L | 220 | 115 | 117 | 118 |
| 40E9-L2H3 | H | 119 | 31 | 32 | 35 |
| | L | 198 | 41 | 42 | 43 |
| 40E9-L2H3-N55S.H | H | 120 | 31 | 121 | 35 |
| | L | 198 | 41 | 42 | 43 |
| 40E9-L2H3-D56G.H | H | 122 | 31 | 123 | 35 |
| | L | 198 | 41 | 42 | 43 |
| 40E9-L2H3-Y101F.H | H | 124 | 31 | 32 | 37 |
| | L | 198 | 41 | 42 | 43 |
| 40E9-L2H3-D105R.H | H | 125 | 31 | 32 | 39 |
| | L | 198 | 41 | 42 | 43 |
| 40E9-L2H6 | H | 126 | 31 | 32 | 127 |
| | L | 198 | 41 | 42 | 43 |
| 40E9-L2H7 | H | 128 | 31 | 129 | 130 |
| | L | 198 | 41 | 42 | 43 |
| 40E9-L2H9 | H | 131 | 31 | 121 | 133 |
| | L | 198 | 41 | 42 | 43 |
| 40E9-L2H10 | H | 134 | 31 | 32 | 135 |
| | L | 198 | 41 | 42 | 43 |
| 40E9-L2H11 | H | 136 | 31 | 32 | 137 |
| | L | 198 | 41 | 42 | 43 |
| 40E9-L2H14 | H | 138 | 31 | 32 | 36 |
| | L | 198 | 41 | 42 | 43 |
| 40E9-L2H16 | H | 139 | 140 | 32 | 35 |
| | L | 198 | 41 | 42 | 43 |
| 40E9-L5H7 | H | 128 | 31 | 129 | 130 |
| | L | 141 | 41 | 42 | 142 |
| mCD3-2C11 | H | 315 | 307 | 308 | 309 |
| | L | 316 | 310 | 311 | 312 |

TABLE 22A
Amino Acid Sequences of CDRs of Exemplary First
Binding Moiety that Binds to CD3

| CDR | SEQ ID NO: | Amino Acid Sequence |
|---|---|---|
| HCDR1 | 31 | SYVIH |
| HCDR2 | 32 | YINPYNDVIKYNEKFKG |
| | 33 | YINPGNDVIKYNEKFKG |
| | 34 | YINPX₁NDVIKYNEKFKG |
| | | (wherein X₁ = Y or G) |
| | 35 | DSYYSYDGFAY |
| | 36 | ESYYSYDGFAY |
| | 37 | DSFYSYDGFAY |
| HCDR3 | 38 | DSYYSYDAFAY |
| | 39 | DSYYSYRGFAY |
| | 40 | X₂SX₃YSYX₄X₅FAY |
| | | (wherein X₂ = D or E, X₃ = Y |
| | | or F, X₄ = D or R, X₅ = G or |
| | | A) |
| LCDR1 | 41 | SASSSVSYMH |
| LCDR2 | 42 | DTSKLAS |
| LCDR3 | 43 | QQWSSNPPT |

TABLE 22B
Amino Acid Sequences of CDRs of Exemplary First
Binding Moiety that Binds to CD3

| CDR | SEQ ID NO: | Amino Acid Sequence |
|---|---|---|
| HCDR1 | 44 | TYAMN |
| | 50 | TYAMN |
| | 182 | DYYIH |
| HCDR2 | 45 | RIRSKYNNYATYYAASVKG |
| | 51 | RIRSKYNNYATYYADSVKG |
| | 183 | WISPGNVNTKYNENFKG |
| HCDR3 | 46 | HGNFGNSYVSWFAY |
| | 52 | HGNFGNSYVSWFAY |
| | 184 | DGYSLYYFDY |
| LCDR1 | 47 | RSSTGAVTTSNYAN |
| | 53 | GSSTGAVTTSNYAN |
| | 185 | KSSQSLLNSRTRKNYLA |
| LCDR2 | 48 | GTNKRAP |
| | 54 | GTNKRAP |
| | 186 | WASTRQS |
| LCDR3 | 49 | ALWYSNLWV |
| | 55 | ALWYSNLWV |
| | 187 | TQSHTLRT |

TABLE 22C
Amino Acid Sequences of CDRs of Exemplary First
Binding Moiety that Binds to CD3

| CDR | SEQ ID NO: | Amino Acid Sequence |
|---|---|---|
| HCDR1 | 96 | SYWMH |
| | 97 | RYWMH |
| | 98 | X₆YWMH |
| | | (wherein X₆ = S or R) |
| HCDR2 | 99 | NINPSNGATNYNEKFKS |
| | 100 | NINPSSGATNYNEKFKS |
| | 101 | NINPRSGATNYNEKFKS |
| | 102 | NINPX₇X₈GATNYNEKFKS |
| | | (wherein X₇ = S or R; X₈ = N |
| | | or S) |
| HCDR3 | 103 | DRSSQYYFDY |
| | 104 | DRSQQYYFDY |
| | 105 | DQSSQYYFDY |
| | 106 | DX₉SX₁₀QYYFDY |
| | | (wherein X₉ = R or Q; X₁₀ = S |
| | | or Q) |
| LCDR1 | 107 | KSSQSLLNSRSRKNYLA |
| | 108 | KSSQSLLNTRSRKNYLA |
| | 109 | KSSQSLLNRRSRKNYLA |
| | 110 | KSSQSLLNSRSRRNYLA |
| | 111 | KSSQSLLYSRSRKNYLA |
| | 112 | KSSQSLLNSRSKKNYLA |
| | 113 | KSSQSLLNSASRKNYLA |
| | 114 | KSSQSLLNSRSSKNYLA |
| | 115 | KSSQSLLNSRSRKKYLA |
| | 116 | KSSQSLLX₁₁X₁₂X₁₃SX₁₄X₁₅X₁₆YLA |
| | | (wherein X₁₁ = N or Y; X₁₂ = |
| | | S, T or R; X₁₃ = R or A; |
| | | X₁₄ = R, K or S; X₁₅ = K or |
| | | R; X₁₆ = N or K) |
| LCDR2 | 117 | WASTRES |
| LCDR3 | 118 | TQSYTLRT |

TABLE 22D
Amino Acid Sequences of CDRs of Exemplary First
Binding Moiety that Binds to CD3

| CDR | SEQ ID NO: | Amino Acid Sequence |
|---|---|---|
| HCDR1 | 140 | DYVIH |
| | 132 | X₂₅YVIH (X₂₅ = S or D) |
| HCDR2 | 121 | YINPYSDVIKYNEKFKG |
| | 123 | YINPYNGVIKYNEKFKG |
| | 129 | YINPGNGVIKYNEKFKG |
| | 143 | YINPX₁₇X₁₈X₁₉VIKYNEKFKG |
| | | (wherein X₁₇ = Y or G, X₁₈ = |
| | | N or S, X₁₉ = D or G |
| HCDR3 | 127 | ESFSSYDGFAY |
| | 130 | DSYSSYDGFAY |
| | 133 | ESFYSYDGFAY |
| | 135 | DSYYSYEAFAY |
| | 137 | DSYYSYRAFAY |
| | 144 | X₂₀SX₂₁X₂₂SYX₂₃X₂₄FAY (wherein |
| | | X₂₀ = D or E, X₂₁ = Y or F, |
| | | X₂₂ = Y or S, X₂₃ = D, R or |
| | | E, X₂₄ = G or A) |
| LCDR1 | 41 | SASSSVSYMH |
| LCDR2 | 42 | DTSKLAS |
| LCDR3 | 142 | QQWRSSPPT |
| | 149 | QQWX₂₆SX₂₇PPT (wherein X₂₆ = |
| | | S or R; X₂₇ = N or S) |

TABLE 22E
Amino Acid Sequences of CDRs of Exemplary First
Binding Moiety that Binds to CD3

| CDR | SEQ ID NO: | Amino Acid Sequence |
|---|---|---|
| HCDR1 | 307 | GYGMH |
| HCDR2 | 308 | YITSSSINIKYADAVKG |
| HCDR3 | 309 | FDWDKNY |
| LCDR1 | 310 | QASQDISNYLN |
| LCDR2 | 311 | YTNKLAD |
| LCDR3 | 312 | QQYYNYPWT |

TABLE 23
Amino Acid Sequences of Each VH and VL of Exemplary First
Binding Moieties that Bind to CD3
VH Region

| SEQ ID NO | Amino Acid Sequence |
|---|---|
| 3 | EVQLQLSGPELVKPGASVKMSCKASGYTFSSYVIHWVKQKP |
| | GQGLEWIGYINPYNDVIKYNEKFKGKATLTSDKSSSTAYMEL |
| | SSLTSEDSAVYYCARDSYYSYDGFAYWGQGTLVTVSA |
| 7 | EVQLVESGGGLVQPGGSLRLSCAASGFTFNTYAMNWVRQAP |
| | GKGLEWVARIRSKYNNYATYYAASVKGRFTISRDDSKNSLY |
| | LQMNSLKTEDTAVYYCARHGNFGNSYVSWFAYWGQGTLVT |
| | VSS |
| 11 | EVQLLESGGGLVQPGGSLRLSCAASGFTFSTYAMNWVRQAP |
| | GKGLEWVSRIRSKYNNYATYYADSVKGRFTISRDDSKNTLY |
| | LQMNSLRAEDTAVYYCVRHGNFGNSYVSWFAYWGQGTLV |
| | TVSS |
| 21 | EVQLVQSGAEVKKPGSSVKVSCKASGGTFSSYVIHWVRQAP |
| | GQGLEWMGYINPYNDVIKYNEKFKGRVTITADKSTSTAYME |
| | LSSLRSEDTAVYYCARDSYYSYDGFAYWGQGTLVTVSS |
| 22 | EVQLVQSGAEVKKPGSSVKVSCKASGYTFSSYVIHWVRQAP |
| | GQGLEWMGYINPYNDVIKYNEKFKGRVTLTSDKSTSTAYME |
| | LSSLRSEDTAVYYCARDSYYSYDGFAYWGQGTLVTVSS |
| 23 | EVQLVQSGAEVKKPGSSVKVSCKASGYTFSSYVIHWVRQAP |
| | GQGLEWIGYINPYNDVIKYNEKFKGRATLTSDKSTSTAYMEL |
| | SSLRSEDTAVYYCARDSYYSYDGFAYWGQGTLVTVSS |
| 26 | EVQLVQSGAEVKKPGSSVKVSCKASGYTFSSYVIHWVRQAP |
| | GQGLEWMGYINPYNDVIKYNEKFKGRVTLTSDKSTSTAYME |
| | LSSLRSEDTAVYYCARESYYSYDGFAYWGQGTLVTVSS |
| 27 | EVQLVQSGAEVKKPGSSVKVSCKASGYTFSSYVIHWVRQAP |
| | GQGLEWMGYINPYNDVIKYNEKFKGRVTLTSDKSTSTAYME |
| | LSSLRSEDTAVYYCARDSFYSYDGFAYWGQGTLVTVSS |
| 28 | EVQLVQSGAEVKKPGSSVKVSCKASGYTFSSYVIHWVRQAP |
| | GQGLEWMGYINPYNDVIKYNEKFKGRVTLTSDKSTSTAYME |
| | LSSLRSEDTAVYYCARDSYYSYDAFAYWGQGTLVTVSS |
| 29 | EVQLVQSGAEVKKPGSSVKVSCKASGYTFSSYVIHWVRQAP |
| | GQGLEWMGYINPGNDVIKYNEKFKGRVTLTSDKSTSTAYME |
| | LSSLRSEDTAVYYCARDSYYSYDGFAYWGQGTLVTVSS |
| 30 | EVQLVQSGAEVKKPGSSVKVSCKASGYTFSSYVIHWVRQAP |
| | GQGLEWMGYINPYNDVIKYNEKFKGRVTLTSDKSTSTAYME |
| | LSSLRSEDTAVYYCARDSYYSYRGFAYWGQGTLVTVSS |
| 80 | QVQLQQPGTELVKPGTSVKLSCKASGYTFTSYWMHWVKQR |
| | PGQGLEWIGNINPSNGATNYNEKFKSKATLTVDKSSSTAYM |
| | QLSSLTSEDSAVYYCARDRSSQYYFDYWGQGTTLTVSS |
| 82 | EVQLVQSGAEVKKPGSSVKVSCKASGYTFTSYWMHWVRQA |
| | PGQGLEWMGNINPSSGATNYNEKFKSRVTLTVDKSTSTAYM |
| | ELSSLRSEDTAVYYCARDRSSQYYFDYWGQGTTVTVSS |
| 83 | EVQLVQSGAEVKKPGSSVKVSCKASGYTFTRYWMHWVRQA |
| | PGQGLEWMGNINPSSGATNYNEKFKSRVTLTVDKSTSTAYM |
| | ELSSLRSEDTAVYYCARDRSSQYYFDYWGQGTTVTVSS |
| 84 | EVQLVQSGAEVKKPGSSVKVSCKASGYTFTSYWMHWVRQA |
| | PGQGLEWMGNINPRSGATNYNEKFKSRVTLTVDKSTSTAYM |
| | ELSSLRSEDTAVYYCARDRSSQYYFDYWGQGTTVTVSS |
| 85 | EVQLVQSGAEVKKPGSSVKVSCKASGYTFTSYWMHWVRQA |
| | PGQGLEWMGNINPSSGATNYNEKFKSRVTLTVDKSTSTAYM |
| | ELSSLRSEDTAVYYCARDRSQQYYFDYWGQGTTVTVSS |
| 86 | EVQLVQSGAEVKKPGSSVKVSCKASGYTFTSYWMHWVRQA |
| | PGQGLEWMGNINPSSGATNYNEKFKSRVTLTVDKSTSTAYM |
| | ELSSLRSEDTAVYYCARDQSSQYYFDYWGQGTTVTVSS |
| 119 | EVQLVQSGAEVKKPGSSVKVSCKASGYTFSSYVIHWVRQAP |
| | GQCLEWMGYINPYNDVIKYNEKFKGRVTLTSDKSTSTAYME |
| | LSSLRSEDTAVYYCARDSYYSYDGFAYWGQGTLVTVSS |
| 120 | EVQLVQSGAEVKKPGSSVKVSCKASGYTFSSYVIHWVRQAP |
| | GQCLEWMGYINPYSDVIKYNEKFKGRVTLTSDKSTSTAYME |
| | LSSLRSEDTAVYYCARDSYYSYDGFAYWGQGTLVTVSS |
| 122 | EVQLVQSGAEVKKPGSSVKVSCKASGYTFSSYVIHWVRQAP |
| | GQCLEWMGYINPYNGVIKYNEKFKGRVTLTSDKSTSTAYME |
| | LSSLRSEDTAVYYCARDSYYSYDGFAYWGQGTLVTVSS |
| 124 | EVQLVQSGAEVKKPGSSVKVSCKASGYTFSSYVIHWVRQAP |
| | GQCLEWMGYINPYNDVIKYNEKFKGRVTLTSDKSTSTAYME |
| | LSSLRSEDTAVYYCARDSFYSYDGFAYWGQGTLVTVSS |
| 125 | EVQLVQSGAEVKKPGSSVKVSCKASGYTFSSYVIHWVRQAP |
| | GQCLEWMGYINPYNDVIKYNEKFKGRVTLTSDKSTSTAYME |
| | LSSLRSEDTAVYYCARDSYYSYRGFAYWGQGTLVTVSS |
| 126 | EVQLVQSGAEVKKPGSSVKVSCKASGYTFSSYVIHWVRQAP |
| | GQCLEWMGYINPYNDVIKYNEKFKGRVTLTSDKSTSTAYME |
| | LSSLRSEDTAVYYCARESFSSYDGFAYWGQGTLVTVSS |
| 128 | EVQLVQSGAEVKKPGSSVKVSCKASGYTFSSYVIHWVRQAP |
| | GQCLEWMGYINPGNGVIKYNEKFKGRVTLTSDKSTSTAYME |
| | LSSLRSEDTAVYYCARDSYSSYDGFAYWGQGTLVTVSS |
| 131 | EVQLVQSGAEVKKPGSSVKVSCKASGYTFSSYVIHWVRQAP |
| | GQCLEWMGYINPYSDVIKYNEKFKGRVTLTSDKSTSTAYME |
| | LSSLRSEDTAVYYCARESFYSYDGFAYWGQGTLVTVSS |
| 134 | EVQLVQSGAEVKKPGSSVKVSCKASGYTFSSYVIHWVRQAP |
| | GQCLEWMGYINPYNDVIKYNEKFKGRVTLTSDKSTSTAYME |
| | LSSLRSEDTAVYYCARDSYYSYEAFAYWGQGTLVTVSS |
| 136 | EVQLVQSGAEVKKPGSSVKVSCKASGYTFSSYVIHWVRQAP |
| | GQCLEWMGYINPYNDVIKYNEKFKGRVTLTSDKSTSTAYME |
| | LSSLRSEDTAVYYCARDSYYSYRAFAYWGQGTLVTVSS |
| 138 | EVQLVQSGAEVKKPGSSVKVSCKASGYTFSSYVIHWVRQAP |
| | GQCLEWMGYINPYNDVIKYNEKFKGRVTLTSDKSTSTAYME |
| | LSSLRSEDTAVYYCARESYYSYDGFAYWGQGTLVTVSS |
| 139 | EVQLVQSGAEVKKPGSSVKVSCKASGYTFSDYVIHWVRQAP |
| | GQCLEWMGYINPYNDVIKYNEKFKGRVTLTSDKSTSTAYME |
| | LSSLRSEDTAVYYCARDSYYSYDGFAYWGQGTLVTVSS |
| 145 | QVQLVQSGAEVKKPGSSVKVSCKASGFAFTDYYIHWVRQAP |
| | GQGLEWMGWISPGNVNTKYNENFKGRVTITADKSTSTAYM |
| | ELSSLRSEDTAVYYCARDGYSLYYFDYWGQGTLVTVSS |
| 195 | EVQLVQSGAEVKKPGSSVKVSCKASGGTFSSYVIHWVRQAP |
| | GQCLEWMGYINPYNDVIKYNEKFKGRVTITADKSTSTAYME |
| | LSSLRSEDTAVYYCARDSYYSYDGFAYWGQGTLVTVSS |
| 196 | EVQLVQSGAEVKKPGSSVKVSCKASGYTFSSYVIHWVRQAP |
| | GQCLEWMGYINPYNDVIKYNEKFKGRVTLTSDKSTSTAYME |
| | LSSLRSEDTAVYYCARDSYYSYDGFAYWGQGTLVTVSS |
| 197 | EVQLVQSGAEVKKPGSSVKVSCKASGYTFSSYVIHWVRQAP |
| | GQCLEWIGYINPYNDVIKYNEKFKGRATLTSDKSTSTAYMEL |
| | SSLRSEDTAVYYCARDSYYSYDGFAYWGQGTLVTVSS |
| 200 | EVQLVQSGAEVKKPGSSVKVSCKASGYTFSSYVIHWVRQAP |
| | GQCLEWMGYINPYNDVIKYNEKFKGRVTLTSDKSTSTAYME |
| | LSSLRSEDTAVYYCARESYYSYDGFAYWGQGTLVTVSS |
| 201 | EVQLVQSGAEVKKPGSSVKVSCKASGYTFSSYVIHWVRQAP |
| | GQCLEWMGYINPYNDVIKYNEKFKGRVTLTSDKSTSTAYME |
| | LSSLRSEDTAVYYCARDSFYSYDGFAYWGQGTLVTVSS |
| 202 | EVQLVQSGAEVKKPGSSVKVSCKASGYTFSSYVIHWVRQAP |
| | GQCLEWMGYINPYNDVIKYNEKFKGRVTLTSDKSTSTAYME |
| | LSSLRSEDTAVYYCARDSYYSYDAFAYWGQGTLVTVSS |
| 203 | EVQLVQSGAEVKKPGSSVKVSCKASGYTFSSYVIHWVRQAP |
| | GQCLEWMGYINPGNDVIKYNEKFKGRVTLTSDKSTSTAYME |
| | LSSLRSEDTAVYYCARDSYYSYDGFAYWGQGTLVTVSS |
| 204 | EVQLVQSGAEVKKPGSSVKVSCKASGYTFSSYVIHWVRQAP |
| | GQCLEWMGYINPYNDVIKYNEKFKGRVTLTSDKSTSTAYME |
| | LSSLRSEDTAVYYCARDSYYSYRGFAYWGQGTLVTVSS |
| 205 | QVQLQQPGTELVKPGTSVKLSCKASGYTFTSYWMHWVKQR |
| | PGQCLEWIGNINPSNGATNYNEKFKSKATLTVDKSSSTAYMQ |
| | LSSLTSEDSAVYYCARDRSSQYYFDYWGQGTTLTVSS |
| 207 | EVQLVQSGAEVKKPGSSVKVSCKASGYTFTSYWMHWVRQA |
| | PGQCLEWMGNINPSSGATNYNEKFKSRVTLTVDKSTSTAYM |
| | ELSSLRSEDTAVYYCARDRSSQYYFDYWGQGTTVTVSS |
| 208 | EVQLVQSGAEVKKPGSSVKVSCKASGYTFTRYWMHWVRQA |
| | PGQCLEWMGNINPSSGATNYNEKFKSRVTLTVDKSTSTAYM |
| | ELSSLRSEDTAVYYCARDRSSQYYFDYWGQGTTVTVSS |
| 209 | EVQLVQSGAEVKKPGSSVKVSCKASGYTFTSYWMHWVRQA |
| | PGQCLEWMGNINPRSGATNYNEKFKSRVTLTVDKSTSTAYM |
| | ELSSLRSEDTAVYYCARDRSSQYYFDYWGQGTTVTVSS |
| 210 | EVQLVQSGAEVKKPGSSVKVSCKASGYTFTSYWMHWVRQA |
| | PGQCLEWMGNINPSSGATNYNEKFKSRVTLTVDKSTSTAYM |
| | ELSSLRSEDTAVYYCARDRSQQYYFDYWGQGTTVTVSS |
| 211 | EVQLVQSGAEVKKPGSSVKVSCKASGYTFTSYWMHWVRQA |
| | PGQCLEWMGNINPSSGATNYNEKFKSRVTLTVDKSTSTAYM |
| | ELSSLRSEDTAVYYCARDQSSQYYFDYWGQGTTVTVSS |
| 221 | EVQLVQSGAEVKKPGSSVKVSCKASGYTFSSYVIHWVRQAP |
| | GQGLEWMGYINPYNDVIKYNEKFKGRVTLTSDKSTSTAYME |
| | LSSLRSEDTAVYYCARDSYYSYDGFAYWGQGTLVTVSS |
| 222 | EVQLVQSGAEVKKPGSSVKVSCKASGYTFSSYVIHWVRQAP |
| | GQGLEWMGYINPYSDVIKYNEKFKGRVTLTSDKSTSTAYME |
| | LSSLRSEDTAVYYCARDSYYSYDGFAYWGQGTLVTVSS |
| 223 | EVQLVQSGAEVKKPGSSVKVSCKASGYTFSSYVIHWVRQAP |
| | GQGLEWMGYINPYNGVIKYNEKFKGRVTLTSDKSTSTAYME |
| | LSSLRSEDTAVYYCARDSYYSYDGFAYWGQGTLVTVSS |
| 224 | EVQLVQSGAEVKKPGSSVKVSCKASGYTFSSYVIHWVRQAP |
| | GQGLEWMGYINPYNDVIKYNEKFKGRVTLTSDKSTSTAYME |
| | LSSLRSEDTAVYYCARDSFYSYDGFAYWGQGTLVTVSS |
| 225 | EVQLVQSGAEVKKPGSSVKVSCKASGYTFSSYVIHWVRQAP |
| | GQGLEWMGYINPYNDVIKYNEKFKGRVTLTSDKSTSTAYME |
| | LSSLRSEDTAVYYCARDSYYSYRGFAYWGQGTLVTVSS |
| 226 | EVQLVQSGAEVKKPGSSVKVSCKASGYTFSSYVIHWVRQAP |
| | GQGLEWMGYINPYNDVIKYNEKFKGRVTLTSDKSTSTAYME |
| | LSSLRSEDTAVYYCARESFSSYDGFAYWGQGTL VTVSS |
| 227 | EVQLVQSGAEVKKPGSSVKVSCKASGYTFSSYVIHWVRQAP |
| | GQGLEWMGYINPGNGVIKYNEKFKGRVTLTSDKSTSTAYME |
| | LSSLRSEDTAVYYCARDSYSSYDGFAYWGQGTLVTVSS |
| 228 | EVQLVQSGAEVKKPGSSVKVSCKASGYTFSSYVIHWVRQAP |
| | GQGLEWMGYINPYSDVIKYNEKFKGRVTLTSDKSTSTAYME |
| | LSSLRSEDTAVYYCARESFYSYDGFAYWGQGTLVTVSS |
| 229 | EVQLVQSGAEVKKPGSSVKVSCKASGYTFSSYVIHWVRQAP |
| | GQGLEWMGYINPYNDVIKYNEKFKGRVTLTSDKSTSTAYME |
| | LSSLRSEDTAVYYCARDSYYSYEAFAYWGQGTLVTVSS |
| 230 | EVQLVQSGAEVKKPGSSVKVSCKASGYTFSSYVIHWVRQAP |
| | GQGLEWMGYINPYNDVIKYNEKFKGRVTLTSDKSTSTAYME |
| | LSSLRSEDTAVYYCARDSYYSYRAFAYWGQGTLVTVSS |
| 231 | EVQLVQSGAEVKKPGSSVKVSCKASGYTFSSYVIHWVRQAP |
| | GQGLEWMGYINPYNDVIKYNEKFKGRVTLTSDKSTSTAYME |
| | LSSLRSEDTAVYYCARESYYSYDGFAYWGQGTLVTVSS |
| 232 | EVQLVQSGAEVKKPGSSVKVSCKASGYTFSDYVIHWVRQAP |
| | GQGLEWMGYINPYNDVIKYNEKFKGRVTLTSDKSTSTAYME |
| | LSSLRSEDTAVYYCARDSYYSYDGFAYWGQGTLVTVSS |
| 315 | EVQLVESGGGLVQPGKSLKLSCEASGFTFSGYGMHWVRQAP |
| | GRCLESVAYITSSSINIKYADAVKGRFTVSRDNAKNLLFLQM |
| | NILKSEDTAMYYCARFDWDKNYWGQGTMVTVSS |

VL Region

| SEQ ID NO | Amino Acid Sequence |
|---|---|
| 4 | QIVLTQSPAIMSASPGEKVTMTCSASSSVSYMHWYQQKSGTS |
| | PKRWIYDTSKLASGVPARFSGSGSGTSYSLTISSMEAEDAATY |
| | YCQQWSSNPPTFGGGTKVEIK |
| 8 | QTVVTQEPSLTVSPGGTVTLTCRSSTGAVTTSNYANWVQQK |
| | PGQAPRGLIGGTNKRAPGTPARFSGSLLGGKAALTLSGVQPE |
| | DEAEYYCALWYSNLWVFGGGTKLTVL |
| 12 | QAVVTQEPSLTVSPGGTVTLTCGSSTGAVTTSNYANWVQEK |
| | PGQAFRGLIGGTNKRAPGTPARFSGSLLGGKAALTLSGAQPE |
| | DEAEYYCALWYSNLWVFGGGTKLTVL |
| 24 | EIVLTQSPDFQSVTPKEKVTITCSASSSVSYMHWYQQKPDQSP |
| | KRLIYDTSKLASGVPSRFSGSGSGTDFTLTINSLEAEDAATYY |
| | CQQWSSNPPTFGGGTKVEIK |
| 25 | EIVLTQSPDFQSVTPKEKVTITCSASSSVSYMHWYQQKPDQSP |
| | KRWIYDTSKLASGVPSRFSGSGSGTDYTLTINSLEAEDAATY |
| | YCQQWSSNPPTFGGGTKVEIK |
| 81 | DIVMSQSPSSLAVSAGEKVTMSCKSSQSLLNSRSRKNYLAWY |
| | QQKPGQSPKLLIYWASTRESGVPDRFTGSGSGTDFTLTISSVQ |
| | AEDLAVYYCTQSYTLRTFGGGTKLEIK |
| 87 | DIVMTQSPDSLAVSLGERATINCKSSQSLLNSRSRKNYLAWY |
| | QQKPGQPPKLLIYWASTRESGVPDRFSGSGSGTDFTLTISSLQ |
| | AEDVAVYYCTQSYTLRTFGGGTKVEIK |
| 88 | DIVMTQSPDSLAVSLGERATINCKSSQSLLNTRSRKNYLAWY |
| | QQKPGQPPKLLIYWASTRESGVPDRFSGSGSGTDFTLTISSLQ |
| | AEDVAVYYCTQSYTLRTFGGGTKVEIK |
| 89 | DIVMTQSPDSLAVSLGERATINCKSSQSLLNRRSRKNYLAWY |
| | QQKPGQPPKLLIYWASTRESGVPDRFSGSGSGTDFTLTISSLQ |
| | AEDVAVYYCTQSYTLRTFGGGTKVEIK |
| 90 | DIVMTQSPDSLAVSLGERATINCKSSQSLLNSRSRRNYLAWY |
| | QQKPGQPPKLLIYWASTRESGVPDRFSGSGSGTDFTLTISSLQ |
| | AEDVAVYYCTQSYTLRTFGGGTKVEIK |
| 91 | DIVMTQSPDSLAVSLGERATINCKSSQSLLYSRSRKNYLAWY |
| | QQKPGQPPKLLIYWASTRESGVPDRFSGSGSGTDFTLTISSLQ |
| | AEDVAVYYCTQSYTLRTFGGGTKVEIK |
| 92 | DIVMTQSPDSLAVSLGERATINCKSSQSLLNSRSKKNYLAWY |
| | QQKPGQPPKLLIYWASTRESGVPDRFSGSGSGTDFTLTISSLQ |
| | AEDVAVYYCTQSYTLRTFGGGTKVEIK |
| 93 | DIVMTQSPDSLAVSLGERATINCKSSQSLLNSASRKNYLAWY |
| | QQKPGQPPKLLIYWASTRESGVPDRFSGSGSGTDFTLTISSLQ |
| | AEDVAVYYCTQSYTLRTFGGGTKVEIK |
| 94 | DIVMTQSPDSLAVSLGERATINCKSSQSLLNSRSSKNYLAWY |
| | QQKPGQPPKLLIYWASTRESGVPDRFSGSGSGTDFTLTISSLQ |
| | AEDVAVYYCTQSYTLRTFGGGTKVEIK |
| 95 | DIVMTQSPDSLAVSLGERATINCKSSQSLLNSRSRKKYLAWY |
| | QQKPGQPPKLLIYWASTRESGVPDRFSGSGSGTDFTLTISSLQ |
| | AEDVAVYYCTQSYTLRTFGGGTKVEIK |
| 198 | EIVLTQSPDFQSVTPKEKVTITCSASSSVSYMHWYQQKPDQSP |
| | KRLIYDTSKLASGVPSRFSGSGSGTDFTLTINSLEAEDAATYY |
| | CQQWSSNPPTFGCGTKVEIK |
| 141 | EIVLTQSPDFQSVTPKEKVTITCSASSSVSYMHWYQQKPDQSP |
| | KRLIYDTSKLASGVPSRFSGSGSGTDFTLTINSLEAEDAATYY |
| | CQQWRSSPPTFGCGTKVEIK |
| 146 | DIVMTQSPDSLAVSLGERATINCKSSQSLLNSRTRKNYLAWY |
| | QQKPGQPPKLLIYWASTRQSGVPDRFSGSGSGTDFTLTISSLQ |
| | AEDVAVYYCTQSHTLRTFGGGTKVEIK |
| 198 | EIVLTQSPDFQSVTPKEKVTITCSASSSVSYMHWYQQKPDQSP |
| | KRLIYDTSKLASGVPSRFSGSGSGTDFTLTINSLEAEDAATYY |
| | CQQWSSNPPTFGCGTKVEIK |
| 199 | EIVLTQSPDFQSVTPKEKVTITCSASSSVSYMHWYQQKPDQSP |
| | KRWIYDTSKLASGVPSRFSGSGSGTDYTLTINSLEAEDAATY |
| | YCQQWSSNPPTFGCGTKVEIK |
| 206 | DIVMSQSPSSLAVSAGEKVTMSCKSSQSLLNSRSRKNYLAWY |
| | QQKPGQSPKLLIYWASTRESGVPDRFTGSGSGTDFTLTISSVQ |
| | AEDLAVYYCTQSYTLRTFGCGTKLEIK |
| 212 | DIVMTQSPDSLAVSLGERATINCKSSQSLLNSRSRKNYLAWY |
| | QQKPGQPPKLLIYWASTRESGVPDRFSGSGSGTDFTLTISSLQ |
| | AEDVAVYYCTQSYTLRTFGCGTKVEIK |
| 213 | DIVMTQSPDSLAVSLGERATINCKSSQSLLNTRSRKNYLAWY |
| | QQKPGQPPKLLIYWASTRESGVPDRFSGSGSGTDFTLTISSLQ |
| | AEDVAVYYCTQSYTLRTFGCGTKVEIK |
| 214 | DIVMTQSPDSLAVSLGERATINCKSSQSLLNRRSRKNYLAWY |
| | QQKPGQPPKLLIYWASTRESGVPDRFSGSGSGTDFTLTISSLQ |
| | AEDVAVYYCTQSYTLRTFGCGTKVEIK |
| 215 | DIVMTQSPDSLAVSLGERATINCKSSQSLLNSRSRRNYLAWY |
| | QQKPGQPPKLLIYWASTRESGVPDRFSGSGSGTDFTLTISSLQ |
| | AEDVAVYYCTQSYTLRTFGCGTKVEIK |
| 216 | DIVMTQSPDSLAVSLGERATINCKSSQSLLYSRSRKNYLAWY |
| | QQKPGQPPKLLIYWASTRESGVPDRFSGSGSGTDFTLTISSLQ |
| | AEDVAVYYCTQSYTLRTFGCGTKVEIK |
| 217 | DIVMTQSPDSLAVSLGERATINCKSSQSLLNSRSKKNYLAWY |
| | QQKPGQPPKLLIYWASTRESGVPDRFSGSGSGTDFTLTISSLQ |
| | AEDVAVYYCTQSYTLRTFGCGTKVEIK |
| 218 | DIVMTQSPDSLAVSLGERATINCKSSQSLLNSASRKNYLAWY |
| | QQKPGQPPKLLIYWASTRESGVPDRFSGSGSGTDFTLTISSLQ |
| | AEDVAVYYCTQSYTLRTFGCGTKVEIK |
| 219 | DIVMTQSPDSLAVSLGERATINCKSSQSLLNSRSSKNYLAWY |
| | QQKPGQPPKLLIYWASTRESGVPDRFSGSGSGTDFTLTISSLQ |
| | AEDVAVYYCTQSYTLRTFGCGTKVEIK |
| 220 | DIVMTQSPDSLAVSLGERATINCKSSQSLLNSRSRKKYLAWY |
| | QQKPGQPPKLLIYWASTRESGVPDRFSGSGSGTDFTLTISSLQ |
| | AEDVAVYYCTQSYTLRTFGCGTKVEIK |
| 233 | EIVLTQSPDFQSVTPKEKVTITCSASSSVSYMHWYQQKPDQSP |
| | KRLIYDTSKLASGVPSRFSGSGSGTDFTLTINSLEAEDAATYY |
| | CQQWSSNPPTFGGGTKVEIK |
| 234 | EIVLTQSPDFQSVTPKEKVTITCSASSSVSYMHWYQQKPDQSP |
| | KRLIYDTSKLASGVPSRFSGSGSGTDFTLTINSLEAEDAATYY |
| | CQQWRSSPPTFGGGTKVEIK |
| 316 | DIQMTQSPSSLPASLGDRVTINCQASQDISNYLNWYQQKPGK |
| | APKLLIYYTNKLADGVPSRFSGSGSGRDSSFTISSLESEDIGSY |
| | YCQQYYNYPWTFGCGTKLEIK |

Given that each of the exemplary first binding moieties can bind to CD3 and that antigen-binding specificity is provided primarily by the CDR1, CDR2 and CDR3 regions, the HCDR1, HCDR2 and HCDR3 sequences and LCDR1, LCDR2 and LCDR3 sequences of each of the exemplary first binding moieties can be “mixed and matched” (i.e., CDRs from different antibodies can be mixed and matched, but each antibody must contain a HCDR1, HCDR2 and HCDR3 and a LCDR1, LCDR2 and LCDR3) to create the first binding moiety of the present disclosure. CD3 binding of such “mixed and matched” binding moieties can be tested using the binding assays described above and in the Examples. Preferably, when VH CDR sequences are mixed and matched, the HCDR1, HCDR2 and/or HCDR3 sequence from a particular VH sequence is replaced with a structurally similar CDR sequence(s). Likewise, when VL CDR sequences are mixed and matched, the LCDR1, LCDR2 and/or LCDR3 sequence from a particular VL sequence preferably is replaced with a structurally similar CDR sequence(s). It will be readily apparent to a person skilled in the art that novel VH and VL sequences can be created by substituting one or more VH and/or VL CDR sequences with structurally similar sequences from the CDR sequences disclosed herein for the exemplary first binding moieties.

CDRs are known to be responsible for antigen binding. However, it has been found that not all of the 6 CDRs are indispensable or unchangeable. In other words, it is possible to replace or change or modify one or more CDRs in each of the exemplary first binding moieties, yet substantially retain the specific binding affinity to CD3.

In certain embodiments, the first binding moieties provided herein comprise a heavy chain CDR3 sequence of one of the anti-CD3 antibodies provided herein. In certain embodiments, the first binding moieties provided herein comprise a heavy chain CDR3 sequence selected from the group consisting of SEQ ID NOs: 35, 36, 37, 38, 39, 40, 46, 52, 103, 104, 105, 106, 127, 130, 133, 135, 137, 144, 184 and 309. Heavy chain CDR3 regions are located at the center of the antigen-binding site, and therefore are believed to make the most contact with antigen and provide the most free energy to the affinity of antibody to antigen. It is also believed that the heavy chain CDR3 is by far the most diverse CDR of the antigen-binding site in terms of length, amino acid composition and conformation by multiple diversification mechanisms (Tonegawa S. Nature. 302:575-81). The diversity in the heavy chain CDR3 is sufficient to produce most antibody specificities (Xu J L, Davis M M. Immunity. 13:37-45) as well as desirable antigen-binding affinity (Schier R, et al., J Mol Biol. 263:551-67).

In certain embodiments, the first binding moieties of the antibodies or antigen-binding fragments thereof provided herein comprise a VH region having an amino acid sequence as set forth in SEQ ID NOs: 3, 7, 11, 21, 22, 23, 26, 27, 28, 29, 30, 80, 82, 83, 84, 85, 86, 119, 120, 122, 124, 125, 126, 128, 131, 134, 136, 138, 139, 145, 195, 196, 197, 200, 201, 202, 203, 204, 205, 207, 208, 209, 210, 211, 221, 222, 223, 224, 225, 226, 227, 228, 229, 230, 231, 232 or 315, or a homologous sequence thereof having at least 80% (e.g., at least 85%, at least 90%, at least 91%, at least 92%, at least 93%, at least 94%, at least 95%, at least 96%, at least 97%, at least 98%, at least 99%) sequence identity to SEQ ID NOs: 3, 7, 11, 21, 22, 23, 26, 27, 28, 29, 30, 80, 82, 83, 84, 85, 86, 119, 120, 122, 124, 125, 126, 128, 131, 134, 136, 138, 139, 145, 195, 196, 197, 200, 201, 202, 203, 204, 205, 207, 208, 209, 210, 211, 221, 222, 223, 224, 225, 226, 227, 228, 229, 230, 231, 232 or 315.

In certain embodiments, the first binding moieties of the antibodies or antigen-binding fragments thereof provided herein comprise a VL region having an amino acid sequence as set forth in SEQ ID NOs: 4, 8, 12, 24, 25, 81, 87, 88, 89, 90, 91, 92, 93, 94, 95, 141, 146, 198, 199, 206, 212, 213, 214, 215, 216, 217, 218, 219, 220, 233, 234 or 316, or a homologous sequence thereof having at least 80% (e.g., at least 85%, at least 90%, at least 91%, at least 92%, at least 93%, at least 94%, at least 95%, at least 96%, at least 97%, at least 98%, at least 99%) sequence identity to SEQ ID NOs: 4, 8, 12, 24, 25, 81, 87, 88, 89, 90, 91, 92, 93, 94, 95, 141, 146, 198, 199, 206, 212, 213, 214, 215, 216, 217, 218, 219, 220, 233, 234 or 316.

In certain embodiments, the first binding moieties of the antibodies or antigen-binding fragments thereof provided herein comprise a VH/VL amino acid sequence pair selected from the group consisting of SEQ ID NOs: 3/4, 7/8, 11/12, 21/24, 22/24, 23/24, 21/25, 26/24, 27/24, 28/24, 29/24, 30/24, 80/81, 83/87, 84/87, 85/87, 86/87, 82/88, 82/89, 82/90, 82/91, 82/92, 82/93, 82/94, 82/95, 119/198, 120/198, 122/198, 124/198, 125/198, 126/198, 128/198, 131/198, 134/198, 136/198, 138/198, 139/198, 128/141, 195/198, 196/198, 197/198, 195/199, 200/198, 201/198, 202/198, 203/198, 204/198, 205/206, 208/212, 209/212, 210/212, 211/212, 207/213, 207/214, 207/215, 207/216, 207/217, 207/218, 207/219, 207/220, 221/233, 222/233, 223/233, 224/233, 225/233, 226/233, 227/233, 227/234, 228/233, 229/233, 230/233, 231/233, 232/233 and 315/316.

In certain embodiments, the first binding moieties of the antibodies or antigen-binding fragments thereof provided herein comprise suitable framework region (FR) sequences, as long as the antibodies and antigen-binding fragments thereof can bind to CD3. The CDR sequences provided in Table 22A, Table 22B, Table 22C, Table 22D and Table 22E above are obtained from chimeric, humanized or human antibodies, but they can be grafted to any suitable FR sequences of any suitable species such as mouse, human, rat, rabbit, among others, using suitable methods known in the art such as recombinant techniques.

In certain embodiments, the first binding moieties of the antibodies or antigen-binding fragments thereof provided herein are humanized. A humanized first binding moiety is desirable in its reduced immunogenicity in human. A humanized antibody is chimeric in its variable regions, as non-human CDR sequences are grafted to human or substantially human FR sequences. Humanization of an antibody or antigen-binding fragment can be essentially performed by substituting the non-human (such as murine) CDR genes for the corresponding human CDR genes in a human immunoglobulin gene (see, for example, Jones et al., (1986) Nature 321:522-525; Riechmann et al., (1988) Nature 332:323-327; Verhoeyen et al., (1988) Science 239:1534-1536).

Suitable human heavy chain and light chain variable domains can be selected to achieve this purpose using methods known in the art. In an illustrative example, “best-fit” approach can be used, where a non-human (e.g., rodent) antibody variable domain sequence is screened or BLASTed against a database of known human variable domain sequences, and the human sequence closest to the non-human query sequence is identified and used as the human scaffold for grafting the non-human CDR sequences (see, for example, Sims et al., (1993) J. Immunol. 151:2296; Chothia et al., (1987) J. Mot. Biol. 196:901). Alternatively, a framework derived from the consensus sequence of all human antibodies may be used for the grafting of the non-human CDRs (see, for example, Carter et al., (1992) Proc. Natl. Acad. Sci. USA, 89:4285; Presta et al., (1993) J. Immunol., 151:2623).

### Second Binding Moiety

In certain embodiments, the first binding moiety that binds to CD3 provided herein is further linked to a second binding moiety having a different specificity from said anti-CD3 first binding moiety. In some embodiments, the bispecific or multi-specific antibody or antigen-binding fragment thereof provided herein has a first specificity for CD3, and a second specificity. In some embodiments, the second specificity is for CD3 but to different epitopes. In some embodiments, the second specificity is for a second antigen different from CD3.

In certain embodiments, the second specificity is for a tumor associated antigen or an epitope thereof. The term “tumor associated antigen” refers to an antigen that is or can be presented on a tumor cell surface and that is located on or within tumor cells. In some embodiments, the tumor associated antigens can be presented only by tumor cells and not by normal cells, i.e., non-tumor cells. In some other embodiments, the tumor associated antigens can be exclusively expressed on tumor cells or may represent a tumor specific mutation compared to non-tumor cells. In some other embodiments, the tumor associated antigens can be found in both tumor cells and non-tumor cells, but are overexpressed on tumor cells when compared to non-tumor cells or are accessible for antibody binding in tumor cells due to the less compact structure of the tumor tissue compared to non-tumor tissue. In some embodiments, the tumor associated antigen is located on the vasculature of a tumor.

In certain embodiments, the bispecific or multi-specific antibodies or antigen-binding fragments thereof provided herein are capable of binding to one or more (e.g., 1, 2, 3, 4, 5 or more) additional antigens other than CD3. In certain embodiments, the one or more additional antigens other than CD3 are selected from the group consisting of GPRC5D, GD2, CD16a, CD33, CD3ε, CD45, CD123, CD146, CD228, CLL-1, FLT3, FLT3L, TAF1, TgPRF, HVCN1, IL-6R, IL-11R, IL17A, IL-23R, IL-33, ILDR2, LAP, TSLP, TREM-1, ANGPT2, APOE, IFNAR, CypA, DOG-1, NKp30, CSF-1R, CCR2, LRRC15, mesothelin, Dickkopf2, DLL3, HER-2, C10orf54, TrkA, MEKK1, KRAS, ERK, XPO1, mTORC1/2, PAK4, NAMPT, ATR, EGFR, FGFR, VEGF, LILRB (e.g., LILRB1, LILRB2, LILRB3, LILRB4, LILRB5), c-MET, Her2, Her3, CTLA4, GITA, CD112R, CD2, CD7, CD16, CD19, CD20, CD24, CD27, CD30, CD34, CD37, CD39, CD70, CD73, CD83, CD28, CD80 (B7-1), CD86 (B7-2), CD40, CD40L (CD154), CD47, SIRPa, CD122, CD137, CD137L, OX40 (CD134), OX40L (CD252), BCMA (e.g., BCMA02), PSMA, CLDN18 (e.g., CLDN18.2), NKG2C, 4-1BB, LIGHT, PVRIG, SLAMF7, HVEM, BAFFR, ICAM-1, 2B4, LFA-1, GITR, ICOS (CD278), ICOSLG (CD275), LAG3 (CD223), A2AR, B7-H3 (CD276), B7-H4 (VTCN1), B7-H5, BTLA (CD272), CD160, CTLA-4 (CD152), IDO (e.g., IDO1, IDO2), ILT3, TDO, KIR, LAIR-1, NOX2, PD-1, PD-L1, PD-L2, TIM-3, VISTA, SIGLEC-7 (CD328), SIGLEC-9 (CD329), SIGLEC-15, TIGIT, PVR (CD155), TLR3, CLEC9A, DEC-205, STING, and TGFB.

In certain embodiments, the bispecific or multi-specific antibody or antigen-binding fragment thereof provided herein comprises a first binding moiety that binds to CD3 (as referred to as “CD3 binding moiety” in the present disclosure) provided herein, and a second binding moiety that binds to GPRC5D (also referred to as “GPRC5D binding moiety” in the present disclosure), wherein the first binding moiety and the second binding moiety are independently a single-chain Fv (scFv) or a Fab domain.

In certain embodiments, the bispecific or multi-specific antibodies or antigen-binding fragments thereof provided herein bind to CD3 and GPRC5D. In some embodiments, the bispecific or multi-specific antibodies or antigen-binding fragments thereof provided herein are in a Fab-scFv-Fab format. In certain embodiments, the bispecific or multi-specific antibodies or antigen-binding fragments thereof provided herein comprise two anti-GPRC5D Fab domains, and one anti-CD3 scFv domain. In certain embodiments, the bispecific or multi-specific antibodies or antigen-binding fragments thereof provided herein comprise two anti-CD3 Fab domains, and one anti-GPRC5D scFv domain.

As used herein, the term “CD3 binding moiety” with regard to a bispecific or multi-specific antibody or antigen-binding fragment thereof, refers to a moiety that is capable of binding to CD3 (e.g., human CD3, mouse CD3, and cynomolgus CD3). The CD3 binding moiety can take any form that allows specific recognition of the target CD3. For example, the CD3 binding moiety may be an antibody or an antigen-binding fragment thereof, e.g., an IgG (such as IgG1, IgG2, IgG3, and IgG4) antibody, IgA antibody, or IgM antibody.

The CD3 binding moiety of the bispecific or multi-specific antibodies provided herein may be derived from any of the bispecific or multi-specific antibodies described above. In certain embodiments, the CD3 binding moiety of the bispecific or multi-specific antibodies provided herein comprises a HCDR1, HCDR2 and/or HCDR3 contained within any one of the heavy chain variable region sequences as set forth in Table 23 above, and a LCDR1, LCDR2 and/or LCDR3 contained within any one of the light chain variable region sequences as set forth in Table 23 above. In certain embodiments, the CD3 binding moiety of the bispecific or multi-specific antibodies provided herein comprises a HCDR1, HCDR2 and/or HCDR3 comprising an amino acid sequence as set forth in Table 22A, Table 22B, Table 22C, Table 22D and Table 22E above. In certain embodiments, the CD3 binding moiety of the bispecific or multi-specific antibodies provided herein comprises a LCDR1, LCDR2 and/or LCDR3 comprising an amino acid sequence as set forth in Table 22A, Table 22B, Table 22C, Table 22D and Table 22E above. In certain embodiments, the CD3 binding moiety of the bispecific or multi-specific antibodies provided herein comprises HCDR1, HCDR2, HCDR3, LCDR1, LCDR2 and LCDR3 comprising an amino acid sequence as set forth in Table 22A, Table 22B, Table 22C, Table 22D and Table 22E above. In certain embodiments, the CD3 binding moiety of the bispecific or multi-specific antibodies provided herein comprises a HCDR1, HCDR2, and/or HCDR3 derived from the illustrative anti-CD3 antibody 40E9, 40E9-L2H1, 40E9-L2H3, 40E9-L2H4, 40E9-L3H1, 40E9-L2H3-D99E.H, 40E9-L2H3-Y101F.H, 40E9-L2H3-G106A.H, 40E9-L2H3-Y54G.H, 40E9-L2H3-D105R.H, 40E9-L2H3-N55S.H, 40E9-L2H3-D56G.H, 40E9-L2H3-D105R.H, 40E9-L2H6, 40E9-L2H7, 40E9-L2H9, 40E9-L2H10, 40E9-L2H11, 40E9-L2H14, 40E9-L2H16, 40E9-L5H7 or mCD3-2C11 as described above. In certain embodiments, the CD3 binding moiety of the bispecific or multi-specific antibodies provided herein comprises a HCDR1, HCDR2, and/or HCDR3 derived from the illustrative anti-CD3 antibody 147E11E2, 147-S31R.H, 147-S54R.H, 147-S102Q.H, 147-R100Q.H, 147-S32T.L, 147-S32R.L, 147-K36R.L, 147-N31Y.L, 147-R35K.L, 147-R33A.L, 147-R35S.L, or 147-N37K.L as described above. In certain embodiments, the CD3 binding moiety of the bispecific or multi-specific antibodies provided herein comprises HCDR1, HCDR2, HCDR3, LCDR1, LCDR2 and LCDR3 derived from the illustrative anti-CD3 antibody 40E9, 40E9-L2H1, 40E9-L2H3, 40E9-L2H4, 40E9-L3H1, 40E9-L2H3-D99E.H, 40E9-L2H3-Y101F.H, 40E9-L2H3-G106A.H, 40E9-L2H3-Y54G.H, 40E9-L2H3-D105R.H, 40E9-L2H3-N55S.H, 40E9-L2H3-D56G.H, 40E9-L2H3-D105R.H, 40E9-L2H6, 40E9-L2H7, 40E9-L2H9, 40E9-L2H10, 40E9-L2H11, 40E9-L2H14, 40E9-L2H16, 40E9-L5H7 or mCD3-2C11 as described above. In certain embodiments, the CD3 binding moiety of the bispecific or multi-specific antibodies provided herein comprises HCDR1, HCDR2, HCDR3, LCDR1, LCDR2 and LCDR3 derived from the illustrative anti-CD3 antibody 147E11E2, 147-S31R.H, 147-S54R.H, 147-S102Q.H, 147-R100Q.H, 147-S32T.L, 147-S32R.L, 147-K36R.L, 147-N31Y.L, 147-R35K.L, 147-R33A.L, 147-R35S.L, or 147-N37K.L as described above.

In certain embodiments, the CD3 binding moiety of the bispecific or multi-specific antibodies provided herein comprises a heavy chain variable region having an amino acid sequence as set forth in Table 23 above, or a homologous sequence having at least 80% (e.g., at least 85%, at least 90%, at least 91%, at least 92%, at least 93%, at least 94%, at least 95%, at least 96%, at least 97%, at least 98%, and at least 99%) sequence identity to the amino acid sequence as set forth in Table 23 above. In certain embodiments, the CD3 binding moiety of the bispecific or multi-specific antibodies provided herein comprises a light chain variable region having an amino acid sequence as set forth in Table 23 above, or a homologous sequence having at least 80% (e.g., at least 85%, at least 90%, at least 91%, at least 92%, at least 93%, at least 94%, at least 95%, at least 96%, at least 97%, at least 98%, and at least 99%) sequence identity to the amino acid sequence as set forth in Table 23 above. In certain embodiments, the CD3 binding moiety of the bispecific or multi-specific antibodies provided herein comprises a VH/VL amino acid sequence pair selected from the group consisting of SEQ ID NOs: 3/4, 7/8, 11/12, 21/24, 22/24, 23/24, 21/25, 26/24, 27/24, 28/24, 29/24, 30/24, 80/81, 83/87, 84/87, 85/87, 86/87, 82/88, 82/89, 82/90, 82/91, 82/92, 82/93, 82/94, 82/95, 119/198, 120/198, 122/198, 124/198, 125/198, 126/198, 128/198, 131/198, 134/198, 136/198, 138/198, 139/198, 128/141, 195/198, 196/198, 197/198, 195/199, 200/198, 201/198, 202/198, 203/198, 204/198, 205/206, 208/212, 209/212, 210/212, 211/212, 207/213, 207/214, 207/215, 207/216, 207/217, 207/218, 207/219, 207/220, 221/233, 222/233, 223/233, 224/233, 225/233, 226/233, 227/233, 227/234, 228/233, 229/233, 230/233, 231/233, 232/233 and 315/316.

As used herein, the term “GPRC5D binding moiety” when referring to a bispecific or multi-specific antibody or antigen-binding fragment thereof, refers to a moiety that is capable of binding to GPRC5D (e.g., human GPRC5D, mouse GPRC5D, and cynomolgus GPRC5D). The GPRC5D binding moiety can take any form that allows specific recognition of the target GPRC5D. For example, the GPRC5D binding moiety may be an antibody or an antigen-binding fragment thereof, e.g., an IgG (such as IgG1, IgG2, IgG3, and IgG4) antibody, IgA antibody, or IgM antibody. In some embodiments, the GPRC5D binding moiety of the bispecific or multi-specific antibodies provided herein is derived from an anti-GPRC5D antibody that binds and activates primary T cells. The GPRC5D binding moiety of the bispecific or multi-specific antibodies provided herein may be derived from any of the anti-GPRC5D antibodies known in the art, e.g., the anti-GPRC5D antibodies described in WO2019154890A1, WO2018147245A1 and US20230192869A1, etc.

In some embodiments, the GPRC5D binding moiety of the bispecific or multi-specific antibodies provided herein comprises one or two or three heavy chain complementarity determining regions (HCDR1, HCDR2 and/or HCDR3) contained within any one of the heavy chain variable (VH) region sequences selected from the group consisting of SEQ ID NO: 1, 5, 9, 13, 14, 15 and 16; and/or one or two or three light chain complementarity determining regions (LCDR1, LCDR2 and/or LCDR3) contained with the light chain variable (VL) region sequences selected from the group consisting of SEQ ID NO: 2, 6, 10, 17 and 18. In some embodiments, the GPRC5D binding moiety of the bispecific or multi-specific antibodies provided herein comprises a homologous sequence thereof having at least 80% (e.g., at least 85%, at least 90%, at least 91%, at least 92%, at least 93%, at least 94%, at least 95%, at least 96%, at least 97%, at least 98%, and at least 99%) sequence identity to SEQ ID NOs: 1, 5, 9, 13, 14, 15 or 16. In some embodiments, the GPRC5D binding moiety of the bispecific or multi-specific antibodies provided herein comprises a homologous sequence thereof having at least 80% (e.g., at least 85%, at least 90%, at least 91%, at least 92%, at least 93%, at least 94%, at least 95%, at least 96%, at least 97%, at least 98%, and at least 99%) sequence identity to SEQ ID NOs: 2, 6, 10, 17 or 18.

In some embodiments, the GPRC5D binding moiety of the bispecific or multi-specific antibodies provided herein comprises HCDR1, HCDR2 and/or HCDR3 contained within the VH region sequence of SEQ ID NO: 1, and LCDR1, LCDR2 and/or LCDR3 contained within the VL region sequence of SEQ ID NO: 2.

In some embodiments, the GPRC5D binding moiety of the bispecific or multi-specific antibodies provided herein comprises HCDR1, HCDR2 and/or HCDR3 contained within the VH region sequence of SEQ ID NO: 5, and LCDR1, LCDR2 and/or LCDR3 contained within the VL region sequence of SEQ ID NO: 6.

In some embodiments, the GPRC5D binding moiety of the bispecific or multi-specific antibodies provided herein comprises HCDR1, HCDR2 and/or HCDR3 contained within the VH region sequence of SEQ ID NO: 9, and LCDR1, LCDR2 and/or LCDR3 contained within the VL region sequence of SEQ ID NO: 10.

In some embodiments, the GPRC5D binding moiety of the bispecific or multi-specific antibodies provided herein comprises HCDR1, HCDR2 and/or HCDR3 contained within the VH region sequence of SEQ ID NO: 15, and LCDR1, LCDR2 and/or LCDR3 contained within the VL region sequence of SEQ ID NO: 18.

In some embodiments, the GPRC5D binding moiety of the bispecific or multi-specific antibodies provided herein comprises HCDR1, HCDR2 and/or HCDR3 contained within the VH region sequence of SEQ ID NO: 13, and LCDR1, LCDR2 and/or LCDR3 contained within the VL region sequence of SEQ ID NO: 18.

In some embodiments, the GPRC5D binding moiety of the bispecific or multi-specific antibodies provided herein comprises HCDR1, HCDR2 and/or HCDR3 contained within the VH region sequence of SEQ ID NO: 16, and LCDR1, LCDR2 and/or LCDR3 contained within the VL region sequence of SEQ ID NO: 17.

In some embodiments, the GPRC5D binding moiety of the bispecific or multi-specific antibodies provided herein comprises HCDR1, HCDR2 and/or HCDR3 contained within the VH region sequence of SEQ ID NO: 14, and LCDR1, LCDR2 and/or LCDR3 contained within the VL region sequence of SEQ ID NO: 18.

In some embodiments, the GPRC5D binding moiety of the bispecific or multi-specific antibodies provided herein comprises at least one heavy or light chain complementarity determining region (CDR) comprising an amino acid sequence selected from the group consisting of SEQ ID NOs: 56, 57, 58, 59, 60, 61, 62, 63, 64, 65, 66, 67, 68, 69, 70, 71, 72 and 73.

In some embodiments, the GPRC5D binding moiety of the bispecific or multi-specific antibodies provided herein comprises one or two or three of HCDR1, HCDR2 and HCDR3 comprising an amino acid sequence selected from the group consisting of SEQ ID NOs: 56, 57, 58, 62, 63, 64, 68, 69 and 70. In some embodiments, the GPRC5D binding moiety of the bispecific or multi-specific antibodies provided herein comprises one or two or three of LCDR1, LCDR2 and LCDR3 comprising an amino acid sequence selected from the group consisting of SEQ ID NOs: 59, 60, 61, 65, 66, 67, 71, 72 and 73.

In some embodiments, the GPRC5D binding moiety of the bispecific or multi-specific antibodies provided herein comprises a HCDR1 comprising an amino acid sequence as set forth in SEQ ID NO: 56, 62 or 68, a HCDR2 comprising an amino acid sequence as set forth in SEQ ID NO: 57, 63 or 69, a HCDR3 comprising an amino acid sequence as set forth in SEQ ID NO: 58, 64 or 70.

In some embodiments, the GPRC5D binding moiety of the bispecific or multi-specific antibodies provided herein comprises a LCDR1 comprising an amino acid sequence as set forth in SEQ ID NO: 59, 65 or 71, a LCDR2 comprising an amino acid sequence as set forth in SEQ ID NO: 60, 66 or 72, a LCDR3 comprising an amino acid sequence as set forth in SEQ ID NO: 61, 67 or 73.

In some embodiments, the GPRC5D binding moiety of the bispecific or multi-specific antibodies provided herein comprises:
i. a HCDR1 comprising an amino acid sequence as set forth in SEQ ID NO: 56, a HCDR2 comprising an amino acid sequence as set forth in SEQ ID NO: 57, and a HCDR3 comprising an amino acid sequence as set forth in SEQ ID NO: 58;
ii. a HCDR1 comprising an amino acid sequence as set forth in SEQ ID NO: 62, a HCDR2 comprising an amino acid sequence as set forth in SEQ ID NO: 63, and a HCDR3 comprising an amino acid sequence as set forth in SEQ ID NO: 64; or
iii. a HCDR1 comprising an amino acid sequence as set forth in SEQ ID NO: 68, a HCDR2 comprising an amino acid sequence as set forth in SEQ ID NO: 69, and a HCDR3 comprising an amino acid sequence as set forth in SEQ ID NO: 70.

In some embodiments, the GPRC5D binding moiety of the bispecific or multi-specific antibodies provided herein comprises:
i a LCDR1 comprising an amino acid sequence as set forth in SEQ ID NO: 59, a LCDR2 comprising an amino acid sequence as set forth in SEQ ID NO: 60, and a LCDR3 comprising an amino acid sequence as set forth in SEQ ID NO: 61;
ii. a LCDR1 comprising an amino acid sequence as set forth in SEQ ID NO: 65, a LCDR2 comprising an amino acid sequence as set forth in SEQ ID NO: 66, and a LCDR3 comprising an amino acid sequence as set forth in SEQ ID NO: 67; or
iii. a LCDR1 comprising an amino acid sequence as set forth in SEQ ID NO: 71, a LCDR2 comprising an amino acid sequence as set forth in SEQ ID NO: 72, and a LCDR3 comprising an amino acid sequence as set forth in SEQ ID NO: 73.

In some embodiments, the GPRC5D binding moiety of the bispecific or multi-specific antibodies provided herein comprises:
i. a HCDR1 comprising an amino acid sequence as set forth in SEQ ID NO: 56, a HCDR2 comprising an amino acid sequence as set forth in SEQ ID NO: 57, and a HCDR3 comprising an amino acid sequence as set forth in SEQ ID NO: 58, a LCDR1 comprising an amino acid sequence as set forth in SEQ ID NO: 59, a LCDR2 comprising an amino acid sequence as set forth in SEQ ID NO: 60, and a LCDR3 comprising an amino acid sequence as set forth in SEQ ID NO: 61;
ii. a HCDR1 comprising an amino acid sequence as set forth in SEQ ID NO: 62, a HCDR2 comprising an amino acid sequence as set forth in SEQ ID NO: 63, and a HCDR3 comprising an amino acid sequence as set forth in SEQ ID NO: 64; a LCDR1 comprising an amino acid sequence as set forth in SEQ ID NO: 65, a LCDR2 comprising an amino acid sequence as set forth in SEQ ID NO: 66, and a LCDR3 comprising an amino acid sequence as set forth in SEQ ID NO: 67; or
iii. a HCDR1 comprising an amino acid sequence as set forth in SEQ ID NO: 68, a HCDR2 comprising an amino acid sequence as set forth in SEQ ID NO: 69, and a HCDR3 comprising an amino acid sequence as set forth in SEQ ID NO: 70, a LCDR1 comprising an amino acid sequence as set forth in SEQ ID NO: 71, a LCDR2 comprising an amino acid sequence as set forth in SEQ ID NO: 72, and a LCDR3 comprising an amino acid sequence as set forth in SEQ ID NO: 73.

In some embodiments, the CDR boundaries of the GPRC5D binding moiety are identified by the convention of Kabat. In some embodiments, the CDR boundaries of the GPRC5D binding moiety are identified by the convention of IMGT. In some embodiments, the CDR boundaries of the GPRC5D binding moiety are identified by the convention of Chothia. In some embodiments, the CDR boundaries of the GPRC5D binding moiety are identified by the convention of Al-Lazikani.

The SEQ ID NOs of the heavy chain (denoted as “H”) variable region, light chain (denoted as “L”) variable region, HCDRs and LCDRs of each of the second binding moiety that binds to GPRC5D described above are shown in Table 24 below. The amino acid sequences of each CDR of the exemplary second binding moieties that binds to GPRC5D are shown in Table 25 below. Unless otherwise indicated, the CDR boundaries as described in Table 24 below were defined or identified by the convention of Kabat. The amino acid sequences of each VH and VL of the exemplary second binding moieties that binds to GPRC5D are shown in Table 26 below.

TABLE 24
SEQ ID NOs of VH, VL, HCDRs and LCDRs of Exemplary
Second Binding Moiety that Binds to GPRC5D

| | | Variable | CDR1 | CDR2 | CDR3 |
|---|---|---|---|---|---|
| | | Region | (SEQ | (SEQ | (SEQ |
| | | (SEQ | ID | ID | ID |
| Antibody | Chain | ID NO) | NO) | NO) | NO) |
| 108C4C12 | H | 1 | 56 | 57 | 58 |
| | L | 2 | 59 | 60 | 61 |
| JNJ-64407564 | H | 5 | 62 | 63 | 64 |
| (anti-GPRC5D moiety) | L | 6 | 65 | 66 | 67 |
| RG6234 (anti-GPRC5D | H | 9 | 68 | 69 | 70 |
| moiety) | L | 10 | 71 | 72 | 73 |
| 108C4C12 | H | 15 | 56 | 57 | 58 |
| VH3-VL4 | L | 18 | 59 | 60 | 61 |
| 108C4C12 | H | 13 | 56 | 57 | 58 |
| VH1-VL4 | L | 18 | 59 | 60 | 61 |
| 108C4C12 | H | 16 | 56 | 57 | 58 |
| VH5-VL3 | L | 17 | 59 | 60 | 61 |
| 108C4C12 | H | 14 | 56 | 57 | 58 |
| VH2-VL4 | L | 18 | 59 | 60 | 61 |

TABLE 25
Amino Acid Sequences of CDRs of Exemplary
Second Binding Moiety that Binds to GPRC5D

| CDR | SEQ ID NO: | Amino Acid Sequence |
|---|---|---|
| HCDR1 | 56 | SYVIH |
| | 62 | GYTMN |
| | 68 | NYGMA |
| HCDR2 | 57 | YFNPYNDGTKYNEEFKG |
| | 63 | LINPYNSDTNYAQKLQG |
| | 69 | SISTGGGNTYYRDSVKG |
| HCDR3 | 58 | GGVRRYFDV |
| | 64 | VALRVALDY |
| | 70 | HDRGGLY |
| LCDR1 | 59 | RASQDIGSNLN |
| | 65 | KASQNVATHVG |
| | 71 | RSSKSLLHSNGITYVY |
| LCDR2 | 60 | ATFGLDS |
| | 66 | SASYRYS |
| | 72 | RMSNLAS |
| LCDR3 | 61 | QQYASFPYT |
| | 67 | QQYNRYPYT |
| | 73 | GQLLENPYT |

TABLE 26
Amino Acid Sequences of Each VH and VL of Exemplary Second
Binding Moiety that Binds to GPRC5D

| Antibody | VH (SEQ ID NO) | VL (SEQ ID NO) |
|---|---|---|
| 108C4C12 | EVQLQQSGPELVKPGASVKMS | DIQMTQSPSSLSASLGERVS |
| | CKASGYTFTSYVIHWVKQKTG | LTCRASQDIGSNLNWLQQEP |
| | QGLEWIGYFNPYNDGTKYNEE | DGTIKRLIYATFGLDSGVPK |
| | FKGKATLTSDKSSNTAYMELN | RFSGSRSGSDYSLTISSLESE |
| | SLTSDDSAVYFCARGGVRRYF | DFVDYYCQQYASFPYTFGG |
| | DVWGAGTTVTVSS | GTKLEIK |
| | (SEQ ID NO: 1) | (SEQ ID NO: 2) |
| JNJ- | QVQLVQSGAEVKKPGASVKV | DIQMTQSPSSLSASVGDRVT |
| 64407564 | SCKASGYSFTGYTMNWVRQA | ITCKASQNVATHVGWYQQK |
| (anti- | PGQGLEWMGLINPYNSDTNYA | PGKAPKRLIYSASYRYSGVP |
| GPRC5D | QKLQGRVTMTTDTSTSTAYME | SRFSGSGSGTEFTLTISNLQP |
| moiety) | LRSLRSDDTAVYYCARVALRV | EDFATYYCQQYNRYPYTFG |
| | ALDYWGQGTLVTVSS | QGTKLEIK |
| | (SEQ ID NO: 5) | (SEQ ID NO: 6) |
| RG6234 | EVQLVESGGGLVQPGRSLKLS | DIVMTQAPLSVSVTPGESAS |
| (anti- | CAASGFSFSNYGMAWVRQAA | ISCRSSKSLLHSNGITYVYW |
| GPRC5D | TKGLEWVASISTGGGNTYYR | YFQKPGKSPQVLIYRMSNL |
| moiety) | DSVKGRFIVSRDNAKNTQYLQ | ASGVPDRFSGSGSETDFTLK |
| | MDSLRSEDTATYYCTRHDRG | ISRVEAEDVGIYHCGQLLEN |
| | GLYWGQGVMVTVSS | PYTFGAGTELELK |
| | (SEQ ID NO: 9) | (SEQ ID NO: 10) |
| 108C4C12 | EVQLVQSGAEVKKPGASVKVS | DIQMTQSPSSLSASVGDRVT |
| VH3-VL4 | CKASGYTFTSYVIHWVRQAPG | ITCRASQDIGSNLNWLQQK |
| | QGLEWMGYFNPYNDGTKYNE | PGKAIKRLIYATFGLDSGVP |
| | EFKGRVTLTSDKSTSTAYMELR | SRFSGSRSGTDFTLTISSLQP |
| | SLRSDDTAVYYCARGGVRRYF | EDFATYYCQQYASFPYTFG |
| | DVWGQGTTVTVSS | GGTKVEIK |
| | (SEQ ID NO: 15) | (SEQ ID NO: 18) |
| 108C4C12 | EVQLVQSGAEVKKPGASVKVS | DIQMTQSPSSLSASVGDRVT |
| VH1-VL4 | CKASGYTFTSYVIHWVRQAPG | ITCRASQDIGSNLNWLQQKP |
| | QGLEWMGYFNPYNDGTKYNE | GKAIKRLIYATFGLDSGVPS |
| | EFKGRVTMTTDTSTSTAYMEL | RFSGSRSGTDFTLTISSLQPE |
| | RSLRSDDTAVYYCARGGVRRY | DFATYYCQQYASFPYTFGGG |
| | FDVWGQGTTVTVSS | TKVEIK |
| | (SEQ ID NO: 13) | (SEQ ID NO: 18) |
| 108C4C12 | EVQLVQSGAEVKKPGASVKVS | DIQMTQSPSSLSASVGDRVT |
| VH5-VL3 | CKASGYTFTSYVIHWVKQAPG | ITCRASQDIGSNLNWLQQKP |
| | QGLEWIGYFNPYNDGTKYNEE | GKAIKRLIYATFGLDSGVPS |
| | FKGKATLTTDKSTSTAYMELRS | RFSGSGSGTDYTLTISSLQPE |
| | LRSDDTAVYYCARGGVRRYFD | DFATYYCQQYASFPYTFGGG |
| | VWGQGTTVTVSS | TKVEIK |
| | (SEQ ID NO: 16) | (SEQ ID NO: 17) |
| 108C4C12 | EVQLVQSGAEVKKPGASVKVS | DIQMTQSPSSLSASVGDRVT |
| VH2-VL4 | CKASGYTFTSYVIHWVRQAPG | ITCRASQDIGSNLNWLQQKP |
| | QGLEWIGYFNPYNDGTKYNEE | GKAIKRLIYATFGLDSGVPS |
| | FKGRATMTTDTSTSTAYMELR | RFSGSRSGTDFTLTISSLQPE |
| | SLRSDDTAVYYCARGGVRRYF | DFATYYCQQYASFPYTFGGG |
| | DVWGQGTTVTVSS | TKVEIK |
| | (SEQ ID NO: 14) | (SEQ ID NO: 18) |

In certain embodiments, the bispecific or multi-specific antibody or antigen-binding fragment thereof provided herein can also comprise a first binding moiety that binds to CD3 (as referred to as “CD3 binding moiety” in the present disclosure) provided herein, and a second binding moiety that binds to GD2 (also referred to as “GD2 binding moiety” in the present disclosure), wherein the first binding moiety and the second binding moiety are independently a single-chain Fv (scFv) or a Fab domain.

In certain embodiments, the bispecific or multi-specific antibodies or antigen-binding fragments thereof provided herein bind to CD3 and GD2. In some embodiments, the bispecific or multi-specific antibodies or antigen-binding fragments thereof provided herein are in a Fab-scFv-Fab format. In certain embodiments, the bispecific or multi-specific antibodies or antigen-binding fragments thereof provided herein comprise two anti-GD2 Fab domains, and one anti-CD3 scFv domain. In certain embodiments, the bispecific or multi-specific antibodies or antigen-binding fragments thereof provided herein comprise two anti-CD3 Fab domains, and one anti-GD2 scFv domain.

As used herein, the term “GD2 binding moiety” when referring to a bispecific or multi-specific antibody or antigen-binding fragment thereof, refers to a moiety that is capable of binding to GD2 (e.g., human GD2, mouse GD2, and cynomolgus GD2). The GD2 binding moiety can take any form that allows specific recognition of the target GD2. For example, the GD2 binding moiety may be an antibody or an antigen-binding fragment thereof, e.g., an IgG (such as IgG1, IgG2, IgG3, and IgG4) antibody, IgA antibody, or IgM antibody. In some embodiments, the GD2 binding moiety of the bispecific or multi-specific antibodies provided herein is derived from an anti-GD2 antibody that binds and activates primary T cells. The GD2 binding moiety of the bispecific or multi-specific antibodies provided herein may be derived from any of the anti-GD2 antibodies known in the art, e.g., the anti-GD2 antibodies described in US7169904B2, US8507657B2 and US9315585B2, etc.

In some embodiments, the GD2 binding moiety of the bispecific or multi-specific antibodies provided herein comprises one or two or three heavy chain complementarity determining regions (HCDR1, HCDR2 and/or HCDR3) contained within the heavy chain variable (VH) region sequence of SEQ ID NO: 19; and/or one or two or three light chain complementarity determining regions (LCDR1, LCDR2 and/or LCDR3) contained with the light chain variable (VL) region sequence of SEQ ID NO: 20. In some embodiments, the GD2 binding moiety of the bispecific or multi-specific antibodies provided herein comprises a homologous sequence thereof having at least 80% (e.g., at least 85%, at least 90%, at least 91%, at least 92%, at least 93%, at least 94%, at least 95%, at least 96%, at least 97%, at least 98%, and at least 99%) sequence identity to SEQ ID NO: 19. In some embodiments, the GD2 binding moiety of the bispecific or multi-specific antibodies provided herein comprises a homologous sequence thereof having at least 80% (e.g., at least 85%, at least 90%, at least 91%, at least 92%, at least 93%, at least 94%, at least 95%, at least 96%, at least 97%, at least 98%, and at least 99%) sequence identity to SEQ ID NO: 20.

In some embodiments, the GD2 binding moiety of the bispecific or multi-specific antibodies provided herein comprises HCDR1, HCDR2 and/or HCDR3 contained within the VH region sequence of SEQ ID NO: 19, and LCDR1, LCDR2 and/or LCDR3 contained within the VL region sequence of SEQ ID NO: 20.

In some embodiments, the GD2 binding moiety of the bispecific or multi-specific antibodies provided herein comprises at least one heavy or light chain complementarity determining region (CDR) comprising an amino acid sequence selected from the group consisting of SEQ ID NOs: 74, 75, 76, 77, 78, and 79.

In some embodiments, the GD2 binding moiety of the bispecific or multi-specific antibodies provided herein comprises one or two or three of HCDR1, HCDR2 and HCDR3 comprising an amino acid sequence selected from the group consisting of SEQ ID NOs: 74, 75, and 76. In some embodiments, the GD2 binding moiety of the bispecific or multi-specific antibodies provided herein comprises one or two or three of LCDR1, LCDR2 and LCDR3 comprising an amino acid sequence selected from the group consisting of SEQ ID NOs: 77, 78, and 79.

In some embodiments, the GD2 binding moiety of the bispecific or multi-specific antibodies provided herein comprises a HCDR1 comprising an amino acid sequence as set forth in SEQ ID NO: 74, a HCDR2 comprising an amino acid sequence as set forth in SEQ ID NO: 75, a HCDR3 comprising an amino acid sequence as set forth in SEQ ID NO: 76; and/or a LCDR1 comprising an amino acid sequence as set forth in SEQ ID NO: 77, a LCDR2 comprising an amino acid sequence as set forth in SEQ ID NO: 78, a LCDR3 comprising an amino acid sequence as set forth in SEQ ID NO: 79.

In some embodiments, the GD2 binding moiety of the bispecific or multi-specific antibodies provided herein comprises a HCDR1 comprising an amino acid sequence as set forth in SEQ ID NO: 74, a HCDR2 comprising an amino acid sequence as set forth in SEQ ID NO: 75, and a HCDR3 comprising an amino acid sequence as set forth in SEQ ID NO: 76.

In some embodiments, the GD2 binding moiety of the bispecific or multi-specific antibodies provided herein comprises a LCDR1 comprising an amino acid sequence as set forth in SEQ ID NO: 77, a LCDR2 comprising an amino acid sequence as set forth in SEQ ID NO: 78, and a LCDR3 comprising an amino acid sequence as set forth in SEQ ID NO: 79.

In some embodiments, the GD2 binding moiety of the bispecific or multi-specific antibodies provided herein comprises a HCDR1 comprising an amino acid sequence as set forth in SEQ ID NO: 74, a HCDR2 comprising an amino acid sequence as set forth in SEQ ID NO: 75, a HCDR3 comprising an amino acid sequence as set forth in SEQ ID NO: 76, a LCDR1 comprising an amino acid sequence as set forth in SEQ ID NO: 77, a LCDR2 comprising an amino acid sequence as set forth in SEQ ID NO: 78, and a LCDR3 comprising an amino acid sequence as set forth in SEQ ID NO: 79.

In some embodiments, the CDR boundaries of the GD2 binding moiety are identified by the convention of Kabat. In some embodiments, the CDR boundaries of the GD2 binding moiety are identified by the convention of IMGT. In some embodiments, the CDR boundaries of the GD2 binding moiety are identified by the convention of Chothia. In some embodiments, the CDR boundaries of the GD2 binding moiety are identified by the convention of Al-Lazikani.

The SEQ ID NOs of the heavy chain (denoted as “H”) variable region, light chain (denoted as “L”) variable region, HCDRs and LCDRs of each of the second binding moiety that binds to GD2 described above are shown in Table 27 below. The amino acid sequences of each CDR of the exemplary second binding moiety that binds to GD2 are shown in Table 28 below. Unless otherwise indicated, the CDR boundaries as described in Table 28 below were defined or identified by the convention of Kabat. The amino acid sequences of each VH and VL of the exemplary second binding moiety that hinds to GD2 are shown in Table 29 below.

TABLE 27
SEQ ID NOs of VH, VL, HCDRs and LCDRs of Exemplary
Second Binding Moiety that Binds to GD2

| | | Variable | CDR1 | CDR2 | CDR3 |
|---|---|---|---|---|---|
| | | Region | (SEQ | (SEQ | (SEQ |
| | | (SEQ | ID | ID | ID |
| Antibody | Chain | ID NO) | NO) | NO) | NO) |
| 3F8 | H | 19 | 74 | 75 | 76 |
| | L | 20 | 77 | 78 | 79 |

TABLE 28
Amino Acid Sequences of CDRs of Exemplary Second
Binding Moiety that Binds to GD2

| CDR | SEQ ID NO: | Amino Acid Sequence |
|---|---|---|
| HCDR1 | 74 | NYGVH |
| HCDR2 | 75 | VIWAGGITNYNSAFMS |
| HCDR3 | 76 | RGGHYGYALDY |
| LCDR1 | 77 | KASQSVSNDVT |
| LCDR2 | 78 | SASNRYS |
| LCDR3 | 79 | QQDYSS |

TABLE 29
Amino Acid Sequences of Each VH and VL of Exemplary Second
Binding Moiety that Binds to GD2

| Antibody | VH (SEQ ID NO) | VL (SEQ ID NO) |
|---|---|---|
| 3F8 | QVQLVESGPGVVQPGRSLRISC | EIVMTQTPATLSVSAGERVTI |
| | AVSGFSVTNYGVHWVRQPPG | TCKASQSVSNDVTWYQQKP |
| | KGLEWLGVIWAGGITNYNSAF | GQAPRLLIYSASNRYSGVPA |
| | MSRLTISKDNSKNTVYLQMNS | RFSGSGYGTEFTFTISSVQSE |
| | LRAEDTAMYYCASRGGHYGY | DFAVYFCQQDYSSFGQGTK |
| | ALDYWGQGTLVTVSS | LEIK |
| | (SEQ ID NO: 19) | (SEQ ID NO: 20) |

An exemplary bispecific antibody or antigen-binding fragment thereof provided herein comprises:
(1) a CD3 binding moiety comprising HCDR1, HCDR2, and HCDR3 contained within VH region sequence as set forth in SEQ ID NO: 7, and LCDR1, LCDR2, and LCDR3 contained within VL region sequence as set forth in SEQ ID NO: 8; and a second binding moiety comprising HCDR1, HCDR2, and HCDR3 contained within VH region sequence as set forth in SEQ ID NO: 1, and LCDR1, LCDR2, and LCDR3 contained within VL region sequence as set forth in SEQ ID NO: 2;
(2) a CD3 binding moiety comprising HCDR1, HCDR2, and HCDR3 contained within VH region sequence as set forth in SEQ ID NO: 7, and LCDR1, LCDR2, and LCDR3 contained within VL region sequence as set forth in SEQ ID NO: 8; and a second binding moiety comprising HCDR1, HCDR2, and HCDR3 contained within VH region sequence as set forth in SEQ ID NO: 9 and LCDR1, LCDR2, and LCDR3 contained within VL region sequence as set forth in SEQ ID NO: 10;
(3) a CD3 binding moiety comprising HCDR1, HCDR2, and HCDR3 contained within VH region sequence as set forth in SEQ ID NO: 7, and LCDR1, LCDR2, and LCDR3 contained within VL region sequence as set forth in SEQ ID NO: 8; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(4) a CD3 binding moiety comprising HCDR1, HCDR2, and HCDR3 contained within VH region sequence as set forth in SEQ ID NO: 7, and LCDR1, LCDR2, and LCDR3 contained within VL region sequence as set forth in SEQ ID NO: 8; and a second binding moiety comprising HCDR1, HCDR2, and HCDR3 contained within VH region sequence as set forth in SEQ ID NO: 13, and LCDR1, LCDR2, and LCDR3 contained within VL region sequence as set forth in SEQ ID NO: 18;
(5) a CD3 binding moiety comprising HCDR1, HCDR2, and HCDR3 contained within VH region sequence as set forth in SEQ ID NO: 7, and LCDR1, LCDR2, and LCDR3 contained within VL region sequence as set forth in SEQ ID NO: 8; and a second binding moiety comprising HCDR1, HCDR2, and HCDR3 contained within VH region sequence as set forth in SEQ ID NO: 16, and LCDR1, LCDR2, and LCDR3 contained within VL region sequence as set forth in SEQ ID NO: 17;
(6) a CD3 binding moiety comprising HCDR1, HCDR2, and HCDR3 contained within VH region sequence as set forth in SEQ ID NO: 7, and LCDR1, LCDR2, and LCDR3 contained within VL region sequence as set forth in SEQ ID NO: 8; and a second binding moiety comprising HCDR1, HCDR2, and HCDR3 contained within VH region sequence as set forth in SEQ ID NO: 14, and LCDR1, LCDR2, and LCDR3 contained within VL region sequence as set forth in SEQ ID NO: 18;
(7) a CD3 binding moiety comprising HCDR1, HCDR2, and HCDR3 contained within VH region sequence as set forth in SEQ ID NO: 3, and LCDR1, LCDR2, and LCDR3 contained within VL region sequence as set forth in SEQ ID NO: 4; and a second binding moiety comprising HCDR1, HCDR2, and HCDR3 contained within VH region sequence as set forth in SEQ ID NO: 1, and LCDR1, LCDR2, and LCDR3 contained within VL region sequence as set forth in SEQ ID NO: 2;
(8) a CD3 binding moiety comprising HCDR1, HCDR2, and HCDR3 contained within VH region sequence as set forth in SEQ ID NO: 21, and LCDR1, LCDR2, and LCDR3 contained within VL region sequence as set forth in SEQ ID NO: 24; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(9) a CD3 binding moiety comprising HCDR1, HCDR2, and HCDR3 contained within VH region sequence as set forth in SEQ ID NO: 22, and LCDR1, LCDR2, and LCDR3 contained within VL region sequence as set forth in SEQ ID NO: 24; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(10) a CD3 binding moiety comprising HCDR1, HCDR2, and HCDR3 contained within VH region sequence as set forth in SEQ ID NO: 23, and LCDR1, LCDR2, and LCDR3 contained within VL region sequence as set forth in SEQ ID NO: 24; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(11) a CD3 binding moiety comprising HCDR1, HCDR2, and HCDR3 contained within VH region sequence as set forth in SEQ ID NO: 26, and LCDR1, LCDR2, and LCDR3 contained within VL region sequence as set forth in SEQ ID NO: 24; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(12) a CD3 binding moiety comprising HCDR1, HCDR2, and HCDR3 contained within VH region sequence as set forth in SEQ ID NO: 27, and LCDR1, LCDR2, and LCDR3 contained within VL region sequence as set forth in SEQ ID NO: 24; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(13) a CD3 binding moiety comprising HCDR1, HCDR2, and HCDR3 contained within VH region sequence as set forth in SEQ ID NO: 28, and LCDR1, LCDR2, and LCDR3 contained within VL region sequence as set forth in SEQ ID NO: 24; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(14) a CD3 binding moiety comprising HCDR1, HCDR2, and HCDR3 contained within VH region sequence as set forth in SEQ ID NO: 29, and LCDR1, LCDR2, and LCDR3 contained within VL region sequence as set forth in SEQ ID NO: 24; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(15) a CD3 binding moiety comprising HCDR1, HCDR2, and HCDR3 contained within VH region sequence as set forth in SEQ ID NO: 30, and LCDR1, LCDR2, and LCDR3 contained within VL region sequence as set forth in SEQ ID NO: 24; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(16) a CD3 binding moiety comprising HCDR1, HCDR2, and HCDR3 contained within VH region sequence as set forth in SEQ ID NO: 21, and LCDR1, LCDR2, and LCDR3 contained within VL region sequence as set forth in SEQ ID NO: 25; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(17) a CD3 binding moiety comprising HCDR1, HCDR2, and HCDR3 contained within VH region sequence as set forth in SEQ ID NO: 80, and LCDR1, LCDR2, and LCDR3 contained within VL region sequence as set forth in SEQ ID NO: 81; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(18) a CD3 binding moiety comprising HCDR1, HCDR2, and HCDR3 contained within VH region sequence as set forth in SEQ ID NO: 83, and LCDR1, LCDR2, and LCDR3 contained within VL region sequence as set forth in SEQ ID NO: 87; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(19) a CD3 binding moiety comprising HCDR1, HCDR2, and HCDR3 contained within VH region sequence as set forth in SEQ ID NO: 84, and LCDR1, LCDR2, and LCDR3 contained within VL region sequence as set forth in SEQ ID NO: 87; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(20) a CD3 binding moiety comprising HCDR1, HCDR2, and HCDR3 contained within VH region sequence as set forth in SEQ ID NO: 85, and LCDR1, LCDR2, and LCDR3 contained within VL region sequence as set forth in SEQ ID NO: 87; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(21) a CD3 binding moiety comprising HCDR1, HCDR2, and HCDR3 contained within VH region sequence as set forth in SEQ ID NO: 86, and LCDR1, LCDR2, and LCDR3 contained within VL region sequence as set forth in SEQ ID NO: 87; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(22) a CD3 binding moiety comprising HCDR1, HCDR2, and HCDR3 contained within VH region sequence as set forth in SEQ ID NO: 82, and LCDR1, LCDR2, and LCDR3 contained within VL region sequence as set forth in SEQ ID NO: 88; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(23) a CD3 binding moiety comprising HCDR1, HCDR2, and HCDR3 contained within VH region sequence as set forth in SEQ ID NO: 82, and LCDR1, LCDR2, and LCDR3 contained within VL region sequence as set forth in SEQ ID NO: 89; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(24) a CD3 binding moiety comprising HCDR1, HCDR2, and HCDR3 contained within VH region sequence as set forth in SEQ ID NO: 82, and LCDR1, LCDR2, and LCDR3 contained within VL region sequence as set forth in SEQ ID NO: 90; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(25) a CD3 binding moiety comprising HCDR1, HCDR2, and HCDR3 contained within VH region sequence as set forth in SEQ ID NO: 82, and LCDR1, LCDR2, and LCDR3 contained within VL region sequence as set forth in SEQ ID NO: 91; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(26) a CD3 binding moiety comprising HCDR1, HCDR2, and HCDR3 contained within VH region sequence as set forth in SEQ ID NO: 82, and LCDR1, LCDR2, and LCDR3 contained within VL region sequence as set forth in SEQ ID NO: 92; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(27) a CD3 binding moiety comprising HCDR1, HCDR2, and HCDR3 contained within VH region sequence as set forth in SEQ ID NO: 82, and LCDR1, LCDR2, and LCDR3 contained within VL region sequence as set forth in SEQ ID NO: 93; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(28) a CD3 binding moiety comprising HCDR1, HCDR2, and HCDR3 contained within VH region sequence as set forth in SEQ ID NO: 82, and LCDR1, LCDR2, and LCDR3 contained within VL region sequence as set forth in SEQ ID NO: 94; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(29) a CD3 binding moiety comprising HCDR1, HCDR2, and HCDR3 contained within VH region sequence as set forth in SEQ ID NO: 82, and LCDR1, LCDR2, and LCDR3 contained within VL region sequence as set forth in SEQ ID NO: 95; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(30) a CD3 binding moiety comprising HCDR1, HCDR2, and HCDR3 contained within VH region sequence as set forth in SEQ ID NO: 3, and LCDR1, LCDR2, and LCDR3 contained within VL region sequence as set forth in SEQ ID NO: 4; and a second binding moiety comprising HCDR1, HCDR2, and HCDR3 contained within VH region sequence as set forth in SEQ ID NO: 19, and LCDR1, LCDR2, and LCDR3 contained within VL region sequence as set forth in SEQ ID NO: 20;
(31) a CD3 binding moiety comprising HCDR1, HCDR2, and HCDR3 contained within VH region sequence as set forth in SEQ ID NO: 195, and LCDR1, LCDR2, and LCDR3 contained within VL region sequence as set forth in SEQ ID NO: 198; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(32) a CD3 binding moiety comprising HCDR1, HCDR2, and HCDR3 contained within VH region sequence as set forth in SEQ ID NO: 196, and LCDR1, LCDR2, and LCDR3 contained within VL region sequence as set forth in SEQ ID NO: 198; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(33) a CD3 binding moiety comprising HCDR1, HCDR2, and HCDR3 contained within VH region sequence as set forth in SEQ ID NO: 197, and LCDR1, LCDR2, and LCDR3 contained within VL region sequence as set forth in and HCDR3 contained within VH region sequence as set forth in SEQ ID NO: 15, and LCDR1, LCDR2, and LCDR3 contained within VL region sequence as set forth in SEQ ID NO: 18;
(34) a CD3 binding moiety comprising HCDR1, HCDR2, and HCDR3 contained within VH region sequence as set forth in SEQ ID NO: 200, and LCDR1, LCDR2, and LCDR3 contained within VL region sequence as set forth in SEQ ID NO: 198; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(35) a CD3 binding moiety comprising HCDR1, HCDR2, and HCDR3 contained within VH region sequence as set forth in SEQ ID NO: 201, and LCDR1, LCDR2, and LCDR3 contained within VL region sequence as set forth in SEQ ID NO: 198; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(36) a CD3 binding moiety comprising HCDR1, HCDR2, and HCDR3 contained within VH region sequence as set forth in SEQ ID NO: 202, and LCDR1, LCDR2, and LCDR3 contained within VL region sequence as set forth in SEQ ID NO: 198; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(37) a CD3 binding moiety comprising HCDR1, HCDR2, and HCDR3 contained within VH region sequence as set forth in SEQ ID NO: 203, and LCDR1, LCDR2, and LCDR3 contained within VL region sequence as set forth in SEQ ID NO: 198; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(38) a CD3 binding moiety comprising HCDR1, HCDR2, and HCDR3 contained within VH region sequence as set forth in SEQ ID NO: 204, and LCDR1, LCDR2, and LCDR3 contained within VL region sequence as set forth in SEQ ID NO: 198; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(39) a CD3 binding moiety comprising HCDR1, HCDR2, and HCDR3 contained within VH region sequence as set forth in SEQ ID NO: 195, and LCDR1, LCDR2, and LCDR3 contained within VL region sequence as set forth in SEQ ID NO: 199; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(40) a CD3 binding moiety comprising HCDR1, HCDR2, and HCDR3 contained within VH region sequence as set forth in SEQ ID NO: 205, and LCDR1, LCDR2, and LCDR3 contained within VL region sequence as set forth in SEQ ID NO: 206; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(41) a CD3 binding moiety comprising HCDR1, HCDR2, and HCDR3 contained within VH region sequence as set forth in SEQ ID NO: 208, and LCDR1, LCDR2, and LCDR3 contained within VL region sequence as set forth in SEQ ID NO: 212; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(42) a CD3 binding moiety comprising HCDR1, HCDR2, and HCDR3 contained within VH region sequence as set forth in SEQ ID NO: 209, and LCDR1, LCDR2, and LCDR3 contained within VL region sequence as set forth in SEQ ID NO: 212; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(43) a CD3 binding moiety comprising HCDR1, HCDR2, and HCDR3 contained within VH region sequence as set forth in SEQ ID NO: 210, and LCDR1, LCDR2, and LCDR3 contained within VL region sequence as set forth in SEQ ID NO: 212; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(44) a CD3 binding moiety comprising HCDR1, HCDR2, and HCDR3 contained within VH region sequence as set forth in SEQ ID NO: 211, and LCDR1, LCDR2, and LCDR3 contained within VL region sequence as set forth in SEQ ID NO: 212; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(45) a CD3 binding moiety comprising HCDR1, HCDR2, and HCDR3 contained within VH region sequence as set forth in SEQ ID NO: 207, and LCDR1, LCDR2, and LCDR3 contained within VL region sequence as set forth in SEQ ID NO: 213; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(46) a CD3 binding moiety comprising HCDR1, HCDR2, and HCDR3 contained within VH region sequence as set forth in SEQ ID NO: 207, and LCDR1, LCDR2, and LCDR3 contained within VL region sequence as set forth in SEQ ID NO: 214; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(47) a CD3 binding moiety comprising HCDR1, HCDR2, and HCDR3 contained within VH region sequence as set forth in SEQ ID NO: 207, and LCDR1, LCDR2, and LCDR3 contained within VL region sequence as set forth in SEQ ID NO: 215; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(48) a CD3 binding moiety comprising HCDR1, HCDR2, and HCDR3 contained within VH region sequence as set forth in SEQ ID NO: 207, and LCDR1, LCDR2, and LCDR3 contained within VL region sequence as set forth in SEQ ID NO: 216; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(49) a CD3 binding moiety comprising HCDR1, HCDR2, and HCDR3 contained within VH region sequence as set forth in SEQ ID NO: 207, and LCDR1, LCDR2, and LCDR3 contained within VL region sequence as set forth in SEQ ID NO: 217; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(50) a CD3 binding moiety comprising HCDR1, HCDR2, and HCDR3 contained within VH region sequence as set forth in SEQ ID NO: 207, and LCDR1, LCDR2, and LCDR3 contained within VL region sequence as set forth in SEQ ID NO: 218; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(51) a CD3 binding moiety comprising HCDR1, HCDR2, and HCDR3 contained within VH region sequence as set forth in SEQ ID NO: 207, and LCDR1, LCDR2, and LCDR3 contained within VL region sequence as set forth in SEQ ID NO: 219; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(52) a CD3 binding moiety comprising HCDR1, HCDR2, and HCDR3 contained within VH region sequence as set forth in SEQ ID NO: 207, and LCDR1, LCDR2, and LCDR3 contained within VL region sequence as set forth in SEQ ID NO: 220; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(53) a CD3 binding moiety comprising HCDR1, HCDR2, and HCDR3 contained within VH region sequence as set forth in SEQ ID NO: 119, and LCDR1, LCDR2, and LCDR3 contained within VL region sequence as set forth in
(54) a CD3 binding moiety comprising HCDR1, HCDR2, and HCDR3 contained within VH region sequence as set forth in SEQ ID NO: 120, and LCDR1, LCDR2, and LCDR3 contained within VL region sequence as set forth in SEQ ID NO: 198; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(55) a CD3 binding moiety comprising HCDR1, HCDR2, and HCDR3 contained within VH region sequence as set forth in SEQ ID NO: 122, and LCDR1, LCDR2, and LCDR3 contained within VL region sequence as set forth in SEQ ID NO: 198; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(56) a CD3 binding moiety comprising HCDR1, HCDR2, and HCDR3 contained within VH region sequence as set forth in SEQ ID NO: 124, and LCDR1, LCDR2, and LCDR3 contained within VL region sequence as set forth in SEQ ID NO: 198; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(57) a CD3 binding moiety comprising HCDR1, HCDR2, and HCDR3 contained within VH region sequence as set forth in SEQ ID NO: 125, and LCDR1, LCDR2, and LCDR3 contained within VL region sequence as set forth in SEQ ID NO: 198; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(58) a CD3 binding moiety comprising HCDR1, HCDR2, and HCDR3 contained within VH region sequence as set forth in SEQ ID NO: 126, and LCDR1, LCDR2, and LCDR3 contained within VL region sequence as set forth in SEQ ID NO: 198; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(59) a CD3 binding moiety comprising HCDR1, HCDR2, and HCDR3 contained within VH region sequence as set forth in SEQ ID NO: 128, and LCDR1, LCDR2, and LCDR3 contained within VL region sequence as set forth in SEQ ID NO: 198; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(60) a CD3 binding moiety comprising HCDR1, HCDR2, and HCDR3 contained within VH region sequence as set forth in SEQ ID NO: 131, and LCDR1, LCDR2, and LCDR3 contained within VL region sequence as set forth in SEQ ID NO: 198; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(61) a CD3 binding moiety comprising HCDR1, HCDR2, and HCDR3 contained within VH region sequence as set forth in SEQ ID NO: 134, and LCDR1, LCDR2, and LCDR3 contained within VL region sequence as set forth in SEQ ID NO: 198; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(62) a CD3 binding moiety comprising HCDR1, HCDR2, and HCDR3 contained within VH region sequence as set forth in SEQ ID NO: 136, and LCDR1, LCDR2, and LCDR3 contained within VL region sequence as set forth in SEQ ID NO: 198; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(63) a CD3 binding moiety comprising HCDR1, HCDR2, and HCDR3 contained within VH region sequence as set forth in SEQ ID NO: 138, and LCDR1, LCDR2, and LCDR3 contained within VL region sequence as set forth in SEQ ID NO: 198; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(64) a CD3 binding moiety comprising HCDR1, HCDR2, and HCDR3 contained within VH region sequence as set forth in SEQ ID NO: 139, and LCDR1, LCDR2, and LCDR3 contained within VL region sequence as set forth in SEQ ID NO: 198; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18;
(65) a CD3 binding moiety comprising HCDR1, HCDR2, and HCDR3 contained within VH region sequence as set forth in SEQ ID NO: 128, and LCDR1, LCDR2, and LCDR3 contained within VL region sequence as set forth in SEQ ID NO: 141; and a second binding moiety comprising HCDR1, HCDR2, and HCDR3 contained within VH region sequence as set forth in SEQ ID NO: 15, and LCDR1, LCDR2, and LCDR3 contained within VL region sequence as set forth in SEQ ID NO: 18; or
(66) a CD3 binding moiety comprising HCDR1, HCDR2, and HCDR3 contained within VH region sequence as set forth in SEQ ID NO: 3, and LCDR1, LCDR2, and LCDR3 contained within VL region sequence as set forth in SEQ ID NO: 4; and a second binding moiety comprising HCDR1, HCDR2, and HCDR3 contained within VH region sequence as set forth in SEQ ID NO: 19, and LCDR1, LCDR2, and LCDR3 contained within VL region sequence as set forth in SEQ ID NO: 20.

An exemplary bispecific antibody or antigen-binding fragment thereof provided herein comprises:
(1) a CD3 binding moiety comprising HCDR1, HCDR2, HCDR3, LCDR1, LCDR2 and LCDR3 as set forth in SEQ ID NOs: 44, 45, 46, 47, 48 and 49, respectively; and a second binding moiety comprising HCDR1, HCDR2, HCDR3, LCDR1, LCDR2 and LCDR3 as set forth in SEQ ID NOs: 56, 57, 58, 59, 60 and 61, respectively;
(2) a CD3 binding moiety comprising HCDR1, HCDR2, HCDR3, LCDR1, LCDR2 and LCDR3 as set forth in SEQ ID NOs: 44, 45, 46, 47, 48 and 49, respectively; and a second binding moiety comprising HCDR1, HCDR2, HCDR3, LCDR1, LCDR2 and LCDR3 as set forth in SEQ ID NOs: 68, 69, 70, 71, 72 and 73, respectively;
(3) a CD3 binding moiety comprising HCDR1, HCDR2, HCDR3, LCDR1, LCDR2 and LCDR3 as set forth in SEQ ID NOs: 44, 45, 46, 47, 48 and 49, respectively; and a second binding moiety comprising HCDR1, HCDR2, HCDR3, LCDR1, LCDR2 and LCDR3 as set forth in SEQ ID NOs: 56, 57, 58, 59, 60 and 61 respectively;
(4) a CD3 binding moiety comprising HCDR1, HCDR2, HCDR3, LCDR1, LCDR2 and LCDR3 as set forth in SEQ ID NOs: 31, 32, 35, 41, 42 and 43, respectively; and a second binding moiety comprising HCDR1, HCDR2, HCDR3, LCDR1, LCDR2 and LCDR3 as set forth in SEQ ID NOs: 56, 57, 58, 59, 60 and 61 respectively;
(5) a CD3 binding moiety comprising HCDR1, HCDR2, HCDR3, LCDR1, LCDR2 and LCDR3 as set forth in SEQ ID NOs: 31, 32, 36, 41, 42 and 43, respectively; and a second binding moiety comprising HCDR1, HCDR2, HCDR3, LCDR1, LCDR2 and LCDR3 as set forth in SEQ ID NOs: 56, 57, 58, 59, 60 and 61 respectively;
(6) a CD3 binding moiety comprising HCDR1, HCDR2, HCDR3, LCDR1, LCDR2 and LCDR3 as set forth in SEQ ID NOs: 31, 32, 37, 41, 42 and 43, respectively; and a second binding moiety comprising HCDR1, HCDR2, HCDR3, LCDR1, LCDR2 and LCDR3 as set forth in SEQ ID NOs: 56, 57, 58, 59, 60 and 61 respectively;
(7) a CD3 binding moiety comprising HCDR1, HCDR2, HCDR3, LCDR1, LCDR2 and LCDR3 as set forth in SEQ ID NOs: 31, 32, 38, 41, 42 and 43, respectively; and a second binding moiety comprising HCDR1, HCDR2, HCDR3, LCDR1, LCDR2 and LCDR3 as set forth in SEQ ID NOs: 56, 57, 58, 59, 60 and 61 respectively;
(8) a CD3 binding moiety comprising HCDR1, HCDR2, HCDR3, LCDR1, LCDR2 and LCDR3 as set forth in SEQ ID NOs: 31, 33, 35, 41, 42 and 43, respectively; and a second binding moiety comprising HCDR1, HCDR2, HCDR3, LCDR1, LCDR2 and LCDR3 as set forth in SEQ ID NOs: 56, 57, 58, 59, 60 and 61 respectively;
(9) a CD3 binding moiety comprising HCDR1, HCDR2, HCDR3, LCDR1, LCDR2 and LCDR3 as set forth in SEQ ID NOs: 31, 32, 39, 41, 42 and 43, respectively; and a second binding moiety comprising HCDR1, HCDR2, HCDR3, LCDR1, LCDR2 and LCDR3 as set forth in SEQ ID NOs: 56, 57, 58, 59, 60 and 61 respectively;
(10) a CD3 binding moiety comprising HCDR1, HCDR2, HCDR3, LCDR1, LCDR2 and LCDR3 as set forth in SEQ ID NOs: 96, 99, 103, 107, 117 and 118, respectively; and a second binding moiety comprising HCDR1, HCDR2, HCDR3, LCDR1, LCDR2 and LCDR3 as set forth in SEQ ID NOs: 56, 57, 58, 59, 60 and 61 respectively;
(11) a CD3 binding moiety comprising HCDR1, HCDR2, HCDR3, LCDR1, LCDR2 and LCDR3 as set forth in SEQ ID NOs: 97, 100, 103, 107, 117 and 118, respectively; and a second binding moiety comprising HCDR1, HCDR2, HCDR3, LCDR1, LCDR2 and LCDR3 as set forth in SEQ ID NOs: 56, 57, 58, 59, 60 and 61 respectively;
(12) a CD3 binding moiety comprising HCDR1, HCDR2, HCDR3, LCDR1, LCDR2 and LCDR3 as set forth in SEQ ID NOs: 96, 101, 103, 107, 117 and 118, respectively; and a second binding moiety comprising HCDR1, HCDR2, HCDR3, LCDR1, LCDR2 and LCDR3 as set forth in SEQ ID NOs: 56, 57, 58, 59, 60 and 61 respectively;
(13) a CD3 binding moiety comprising HCDR1, HCDR2, HCDR3, LCDR1, LCDR2 and LCDR3 as set forth in SEQ ID NOs: 96, 100, 104, 107, 117 and 118, respectively; and a second binding moiety comprising HCDR1, HCDR2, HCDR3, LCDR1, LCDR2 and LCDR3 as set forth in SEQ ID NOs: 56, 57, 58, 59, 60 and 61 respectively;
(14) a CD3 binding moiety comprising HCDR1, HCDR2, HCDR3, LCDR1, LCDR2 and LCDR3 as set forth in SEQ ID NOs: 96, 100, 105, 107, 117 and 118, respectively; and a second binding moiety comprising HCDR1, HCDR2, HCDR3, LCDR1, LCDR2 and LCDR3 as set forth in SEQ ID NOs: 56, 57, 58, 59, 60 and 61 respectively;
(15) a CD3 binding moiety comprising HCDR1, HCDR2, HCDR3, LCDR1, LCDR2 and LCDR3 as set forth in SEQ ID NOs: 96, 100, 103, 108, 117 and 118, respectively; and a second binding moiety comprising HCDR1, HCDR2, HCDR3, LCDR1, LCDR2 and LCDR3 as set forth in SEQ ID NOs: 56, 57, 58, 59, 60 and 61 respectively;
(16) a CD3 binding moiety comprising HCDR1, HCDR2, HCDR3, LCDR1, LCDR2 and LCDR3 as set forth in SEQ ID NOs: 96, 100, 103, 109, 117 and 118, respectively; and a second binding moiety comprising HCDR1, HCDR2, HCDR3, LCDR1, LCDR2 and LCDR3 as set forth in SEQ ID NOs: 56, 57, 58, 59, 60 and 61 respectively;
(17) a CD3 binding moiety comprising HCDR1, HCDR2, HCDR3, LCDR1, LCDR2 and LCDR3 as set forth in SEQ ID NOs: 96, 100, 103, 110, 117 and 118, respectively; and a second binding moiety comprising HCDR1, HCDR2, HCDR3, LCDR1, LCDR2 and LCDR3 as set forth in SEQ ID NOs: 56, 57, 58, 59, 60 and 61 respectively;
(18) a CD3 binding moiety comprising HCDR1, HCDR2, HCDR3, LCDR1, LCDR2 and LCDR3 as set forth in SEQ ID NOs: 96, 100, 103, 111, 117 and 118, respectively; and a second binding moiety comprising HCDR1, HCDR2, HCDR3, LCDR1, LCDR2 and LCDR3 as set forth in SEQ ID NOs: 56, 57, 58, 59, 60 and 61 respectively;
(19) a CD3 binding moiety comprising HCDR1, HCDR2, HCDR3, LCDR1, LCDR2 and LCDR3 as set forth in SEQ ID NOs: 96, 100, 103, 112, 117 and 118, respectively; and a second binding moiety comprising HCDR1, HCDR2, HCDR3, LCDR1, LCDR2 and LCDR3 as set forth in SEQ ID NOs: 56, 57, 58, 59, 60 and 61 respectively;
(20) a CD3 binding moiety comprising HCDR1, HCDR2, HCDR3, LCDR1, LCDR2 and LCDR3 as set forth in SEQ ID NOs: 96, 100, 103, 113, 117 and 118, respectively; and a second binding moiety comprising HCDR1, HCDR2, HCDR3, LCDR1, LCDR2 and LCDR3 as set forth in SEQ ID NOs: 56, 57, 58, 59, 60 and 61 respectively;
(21) a CD3 binding moiety comprising HCDR1, HCDR2, HCDR3, LCDR1, LCDR2 and LCDR3 as set forth in SEQ ID NOs: 96, 100, 103, 114, 117 and 118, respectively; and a second binding moiety comprising HCDR1, HCDR2, HCDR3, LCDR1, LCDR2 and LCDR3 as set forth in SEQ ID NOs: 56, 57, 58, 59, 60 and 61 respectively; or
(22) a CD3 binding moiety comprising HCDR1, HCDR2, HCDR3, LCDR1, LCDR2 and LCDR3 as set forth in SEQ ID NOs: 96, 100, 103, 115, 117 and 118, respectively; and a second binding moiety comprising HCDR1, HCDR2, HCDR3, LCDR1, LCDR2 and LCDR3 as set forth in SEQ ID NOs: 56, 57, 58, 59, 60 and 61 respectively.

In certain embodiments, the bispecific or multi-specific antibody or antigen-binding fragment thereof provided herein can also comprise a first binding moiety that binds to CD3 (as referred to as “CD3 binding moiety” in the present disclosure) provided herein, and a second binding moiety that binds to LILRB4 (also referred to as “LILRB4 binding moiety” in the present disclosure), wherein the first binding moiety and the second binding moiety are independently a single-chain Fv (scFv) or a Fab domain.

In certain embodiments, the bispecific or multi-specific antibodies or antigen-binding fragments thereof provided herein bind to CD3 and LILRB4. In some embodiments, the bispecific or multi-specific antibodies or antigen-binding fragments thereof provided herein are in a Fab-scFv-Fab format. In certain embodiments, the bispecific or multi-specific antibodies or antigen-binding fragments thereof provided herein comprise two anti-LILRB4 Fab domains, and one anti-CD3 scFv domain. In certain embodiments, the bispecific or multi-specific antibodies or antigen-binding fragments thereof provided herein comprise two anti-CD3 Fab domains, and one anti-LILRB4 scFv domain.

As used herein, the term “LILRB4 binding moiety” when referring to a bispecific or multi-specific antibody or antigen-binding fragment thereof, refers to a moiety that is capable of binding to LILRB4 (e.g., human LILRB4, mouse LILRB4, or cynomolgus LILRB4). The LILRB4 binding moiety can take any form that allows specific recognition of the target LILRB4. For example, the LILRB4 binding moiety may be an antibody or an antigen-binding fragment thereof, e.g., an IgG (such as IgG1, IgG2, IgG3, and IgG4) antibody, IgA antibody, or IgM antibody. In some embodiments, the LILRB4 binding moiety of the bispecific or multi-specific antibodies provided herein is derived from an anti-LILRB4 antibody. The LILRB4 binding moiety of the bispecific or multi-specific antibodies provided herein may be derived from any of the anti-LILRB4 antibodies known in the art, e.g., the anti-LILRB4 antibodies described in WO2020180789A1, WO2021183839A2, etc.

In some embodiments, the LILRB4 binding moiety of the bispecific or multi-specific antibodies provided herein comprises one or two or three heavy chain complementarity determining regions (HCDR1, HCDR2 and/or HCDR3) contained within the heavy chain variable (VH) region sequence of SEQ ID NO: 147; and/or one or two or three light chain complementarity determining regions (LCDR1, LCDR2 and/or LCDR3) contained with the light chain variable (VL) region sequence of SEQ ID NO: 148. In some embodiments, the LILRB4 binding moiety of the bispecific or multi-specific antibodies provided herein comprises a homologous sequence thereof having at least 80% (e.g., at least 85%, at least 90%, at least 91%, at least 92%, at least 93%, at least 94%, at least 95%, at least 96%, at least 97%, at least 98%, and at least 99%) sequence identity to SEQ ID NO: 147. In some embodiments, the LILRB4 binding moiety of the bispecific or multi-specific antibodies provided herein comprises a homologous sequence thereof having at least 80% (e.g., at least 85%, at least 90%, at least 91%, at least 92%, at least 93%, at least 94%, at least 95%, at least 96%, at least 97%, at least 98%, and at least 99%) sequence identity to SEQ ID NO: 148.

In some embodiments, the LILRB4 binding moiety of the bispecific or multi-specific antibodies provided herein comprises HCDR1, HCDR2 and/or HCDR3 contained within the VH region sequence of SEQ ID NO: 147, and LCDR1, LCDR2 and/or LCDR3 contained within the VL region sequence of SEQ ID NO: 148.

In some embodiments, the LILRB4 binding moiety of the bispecific or multi-specific antibodies provided herein comprises at least one heavy or light chain complementarity determining region (CDR) comprising an amino acid sequence selected from the group consisting of SEQ ID NOs: 188, 189, 190, 191, 192, and 193.

In some embodiments, the LILRB4 binding moiety of the bispecific or multi-specific antibodies provided herein comprises one or two or three of HCDR1, HCDR2 and HCDR3 comprising an amino acid sequence selected from the group consisting of SEQ ID NOs: 188, 189, and 190. In some embodiments, the LILRB4 binding moiety of the bispecific or multi-specific antibodies provided herein comprises one or two or three of LCDR1, LCDR2 and LCDR3 comprising an amino acid sequence selected from the group consisting of SEQ ID NOs: 191, 192, and 193.

In some embodiments, the LILRB4 binding moiety of the bispecific or multi-specific antibodies provided herein comprises a HCDR1 comprising an amino acid sequence as set forth in SEQ ID NO: 188, a HCDR2 comprising an amino acid sequence as set forth in SEQ ID NO: 189, a HCDR3 comprising an amino acid sequence as set forth in SEQ ID NO: 190; and/or a LCDR1 comprising an amino acid sequence as set forth in SEQ ID NO: 191, a LCDR2 comprising an amino acid sequence as set forth in SEQ ID NO: 192, a LCDR3 comprising an amino acid sequence as set forth in SEQ ID NO: 193.

In some embodiments, the LILRB4 binding moiety of the bispecific or multi-specific antibodies provided herein comprises a HCDR1 comprising an amino acid sequence as set forth in SEQ ID NO: 188, a HCDR2 comprising an amino acid sequence as set forth in SEQ ID NO: 189, and a HCDR3 comprising an amino acid sequence as set forth in SEQ ID NO: 190.

In some embodiments, the LILRB4 binding moiety of the bispecific or multi-specific antibodies provided herein comprises a LCDR1 comprising an amino acid sequence as set forth in SEQ ID NO: 191, a LCDR2 comprising an amino acid sequence as set forth in SEQ ID NO: 192, and a LCDR3 comprising an amino acid sequence as set forth in SEQ ID NO: 193.

In some embodiments, the LILRB4 binding moiety of the bispecific or multi-specific antibodies provided herein comprises a HCDR1 comprising an amino acid sequence as set forth in SEQ ID NO: 188, a HCDR2 comprising an amino acid sequence as set forth in SEQ ID NO: 189, a HCDR3 comprising an amino acid sequence as set forth in SEQ ID NO: 190, a LCDR1 comprising an amino acid sequence as set forth in SEQ ID NO: 191, a LCDR2 comprising an amino acid sequence as set forth in SEQ ID NO: 192, and a LCDR3 comprising an amino acid sequence as set forth in SEQ ID NO: 193.

In some embodiments, the LILRB4 binding moiety of the bispecific or multi-specific antibodies provided herein comprises one or two or three heavy chain complementarity determining regions (HCDR1, HCDR2 and/or HCDR3) contained within the heavy chain variable (VH) region sequence of SEQ ID NO: 243; and/or one or two or three light chain complementarity determining regions (LCDR1, LCDR2 and/or LCDR3) contained with the light chain variable (VL) region sequence of SEQ ID NO: 247. In some embodiments, the LILRB4 binding moiety of the bispecific or multi-specific antibodies provided herein comprises a homologous sequence thereof having at least 80% (e.g., at least 85%, at least 90%, at least 91%, at least 92%, at least 93%, at least 94%, at least 95%, at least 96%, at least 97%, at least 98%, and at least 99%) sequence identity to SEQ ID NO: 243. In some embodiments, the LILRB4 binding moiety of the bispecific or multi-specific antibodies provided herein comprises a homologous sequence thereof having at least 80% (e.g., at least 85%, at least 90%, at least 91%, at least 92%, at least 93%, at least 94%, at least 95%, at least 96%, at least 97%, at least 98%, and at least 99%) sequence identity to SEQ ID NO: 247.

In some embodiments, the LILRB4 binding moiety of the bispecific or multi-specific antibodies provided herein comprises HCDR1, HCDR2 and/or HCDR3 contained within the VH region sequence of SEQ ID NO: 243, and LCDR1, LCDR2 and/or LCDR3 contained within the VL region sequence of SEQ ID NO: 247.

In some embodiments, the LILRB4 binding moiety of the bispecific or multi-specific antibodies provided herein comprises at least one heavy or light chain complementarity determining region (CDR) comprising an amino acid sequence selected from the group consisting of SEQ ID NOs: 244, 245, 246, 248, 249, and 250.

In some embodiments, the LILRB4 binding moiety of the bispecific or multi-specific antibodies provided herein comprises one or two or three of HCDR1, HCDR2 and HCDR3 comprising an amino acid sequence selected from the group consisting of SEQ ID NOs: 244, 245, and 246. In some embodiments, the LILRB4 binding moiety of the bispecific or multi-specific antibodies provided herein comprises one or two or three of LCDR1, LCDR2 and LCDR3 comprising an amino acid sequence selected from the group consisting of SEQ ID NOs: 248, 249, and 250.

In some embodiments, the LILRB4 binding moiety of the bispecific or multi-specific antibodies provided herein comprises a HCDR1 comprising an amino acid sequence as set forth in SEQ ID NO: 244, a HCDR2 comprising an amino acid sequence as set forth in SEQ ID NO: 245, a HCDR3 comprising an amino acid sequence as set forth in SEQ ID NO: 246; and/or a LCDR1 comprising an amino acid sequence as set forth in SEQ ID NO: 248, a LCDR2 comprising an amino acid sequence as set forth in SEQ ID NO: 249, a LCDR3 comprising an amino acid sequence as set forth in SEQ ID NO: 250.

In some embodiments, the LILRB4 binding moiety of the bispecific or multi-specific antibodies provided herein comprises a HCDR1 comprising an amino acid sequence as set forth in SEQ ID NO: 244, a HCDR2 comprising an amino acid sequence as set forth in SEQ ID NO: 245, and a HCDR3 comprising an amino acid sequence as set forth in SEQ ID NO: 246.

In some embodiments, the LILRB4 binding moiety of the bispecific or multi-specific antibodies provided herein comprises a LCDR1 comprising an amino acid sequence as set forth in SEQ ID NO: 248, a LCDR2 comprising an amino acid sequence as set forth in SEQ ID NO: 249, and a LCDR3 comprising an amino acid sequence as set forth in SEQ ID NO: 250.

In some embodiments, the LILRB4 binding moiety of the bispecific or multi-specific antibodies provided herein comprises a HCDR1 comprising an amino acid sequence as set forth in SEQ ID NO: 244, a HCDR2 comprising an amino acid sequence as set forth in SEQ ID NO: 245, a HCDR3 comprising an amino acid sequence as set forth in SEQ ID NO: 246, a LCDR1 comprising an amino acid sequence as set forth in SEQ ID NO: 248, a LCDR2 comprising an amino acid sequence as set forth in SEQ ID NO: 249, and a LCDR3 comprising an amino acid sequence as set forth in SEQ ID NO: 250.

In some embodiments, the CDR boundaries of the LILRB4 binding moiety are identified by the convention of Kabat. In some embodiments, the CDR boundaries of the LILRB4 binding moiety are identified by the convention of IMGT. In some embodiments, the CDR boundaries of the LILRB4 binding moiety are identified by the convention of Chothia. In some embodiments, the CDR boundaries of the LILRB4 binding moiety are identified by the convention of Al-Lazikani.

The SEQ ID NOs of the heavy chain (denoted as “H”) variable region, light chain (denoted as “L”) variable region, HCDRs and LCDRs of each of the second binding moiety that binds to LILRB4 described above are shown in Table 45 below. The amino acid sequences of each CDR of the exemplary second binding moiety that binds to LILRB4 are shown in Table 46 below. Unless otherwise indicated, the CDR boundaries as described in Table 46 below were defined or identified by the convention of Kabat. The amino acid sequences of each VH and VL of the exemplary second binding moiety that binds to LILRB4 are shown in Table 47 below.

TABLE 45
SEQ ID NOs of VH, VL, HCDRs and LCDRs of Exemplary
Second Binding Moiety that Binds to LILRB4

| | | Variable | CDR1 | CDR2 | CDR3 |
|---|---|---|---|---|---|
| | | Region | (SEQ | (SEQ | (SEQ |
| | | (SEQ | ID | ID | ID |
| Antibody | Chain | ID NO) | NO) | NO) | NO) |
| IO-312 (anti- | H | 147 | 188 | 189 | 190 |
| LILRB4 moiety) | L | 148 | 191 | 192 | 193 |
| PTMS16-22 (anti- | H | 243 | 244 | 245 | 246 |
| LILRB4 moiety) | L | 247 | 248 | 249 | 250 |

TABLE 46
Amino Acid Sequences of CDRs of Exemplary Second
Binding Moiety that Binds to LILRB4

| CDR | SEQ ID NO | Amino Acid Sequence |
|---|---|---|
| HCDR1 | 188 | SSYWIS |
| | 244 | YWMS |
| HCDR2 | 189 | SIDSGSVGITYYATWVKG |
| | 245 | EINPESSAINYAPSLKD |
| HCDR3 | 190 | HGDNWALDL |
| | 246 | SMTMDY |
| LCDR1 | 191 | ASQSISSWLA |
| | 248 | KASQDIKSYLS |
| LCDR2 | 192 | KASTLAS |
| | 249 | YATSLAG |
| LCDR3 | 193 | QHGYIRGDLDNV |
| | 250 | LQHGESPYT |

TABLE 47
Amino Acid Sequences of Each VH and VL of Exemplary Second
Binding Moiety that Binds to LILRB4

| Antibody | VH (SEQ ID NO) | VL (SEQ ID NO) |
|---|---|---|
| IO-312 | EVQLVESGGGLVQPGGSLRLS | DIQMTQSPSTLSASVGDRVT |
| (anti- | CAASGFSLSSSYWISWVRQAP | ITCRASQSISSWLAWYQQK |
| LILRB4 | GKGLEWIGSIDSGSVGITYYA | PGKAPKLLIYKASTLASGVP |
| moiety) | TWVKGRFTISRDNSKNTLYLQ | SRFSGSGSGTEFTLTISSLQP |
| | MNSLRAEDTAVYYCARHGDN | DDFATYYCQHGYIRGDLD |
| | WALDLWGQGTLVTVLE | NVFGGGTKVEIK |
| | (SEQ ID NO: 147) | (SEQ ID NO: 148) |
| PTMS16- | EVQLVESGGGLVQPGGSLRLS | DIQMTQSPSSLSASVGDRVT |
| 22 (anti- | CAASGFDFSRYWMSWVRQAP | ITCKASQDIKSYLSWYQQK |
| LILRB4 | GKGLVWIGEINPESSAINYAPS | PGKAPKTLIYYATSLAGGV |
| moiety) | LKDRFTISRDNAKNTLYLQMN | PSRFSGSGSGTDYTLTISSLQ |
| | SLRAEDTAVYYCATSMTMDY | PEDFATYYCLQHGESPYTF |
| | WGQGTLVTVSS | GGGTKLEIK |
| | (SEQ ID NO: 243) | (SEQ ID NO: 247) |

An exemplary bispecific antibody or antigen-binding fragment thereof provided herein comprises:
(1) a CD3 binding moiety comprising HCDR1, HCDR2, and HCDR3 contained within VH region sequence as set forth in SEQ ID NO: 145, and LCDR1, LCDR2, and LCDR3 contained within VL region sequence as set forth in SEQ ID NO: 146; and a second binding moiety comprising HCDR1, HCDR2, NO: 147, and LCDR1, LCDR2, and LCDR3 contained within VL region sequence as set forth in SEQ ID NO: 148; or
(2) a CD3 binding moiety comprising HCDR1, HCDR2, and HCDR3 contained within VH region sequence as set forth in SEQ ID NO: 145, and LCDR1, LCDR2, and LCDR3 contained within VL region sequence as set forth in SEQ ID NO: 146; and a second binding moiety comprising HCDR1, HCDR2, and HCDR3 contained within VH region sequence as set forth in SEQ ID NO: 243, and LCDR1, LCDR2, and LCDR3 contained within VL region sequence as set forth in SEQ ID NO: 247.

In certain embodiments, the bispecific or multi-specific antibody or antigen-binding fragment thereof provided herein can also comprise a first binding moiety that binds to CD3 (as referred to as “CD3 binding moiety” in the present disclosure) provided herein, and a second binding moiety that binds to CD19 (also referred to as “CD19 binding moiety” in the present disclosure), wherein the first binding moiety and the second binding moiety are independently a single-chain Fv (scFv) or a Fab domain.

In certain embodiments, the bispecific or multi-specific antibodies or antigen-binding fragments thereof provided herein bind to CD3 and CD19. In some embodiments, the bispecific or multi-specific antibodies or antigen-binding fragments thereof provided herein are in a Fab-scFv-Fab format. In certain embodiments, the bispecific or multi-specific antibodies or antigen-binding fragments thereof provided herein comprise two anti-CD19 Fab domains, and one anti-CD3 scFv domain. In certain embodiments, the bispecific or multi-specific antibodies or antigen-binding fragments thereof provided herein comprise two anti-CD3 Fab domains, and one anti-CD19 scFv domain.

As used herein, the term “CD19 binding moiety” when referring to a bispecific or multi-specific antibody or antigen-binding fragment thereof, refers to a moiety that is capable of binding to CD19 (e.g., human CD19, mouse CD19, or cynomolgus CD19). The CD19 binding moiety can take any form that allows specific recognition of the target CD19. For example, the CD19 binding moiety may be an antibody or an antigen-binding fragment thereof, e.g., an IgG (such as IgG1, IgG2, IgG3, and IgG4) antibody, IgA antibody, or IgM antibody. In some embodiments, the CD19 binding moiety of the bispecific or multi-specific antibodies provided herein is derived from an anti-CD19 antibody. The CD19 binding moiety of the bispecific or multi-specific antibodies provided herein may be derived from any of the anti-CD19 antibodies known in the art, e.g., the anti-CD19 antibodies described in WO2020210232A1, WO2021173471A1, etc.

In some embodiments, the CD19 binding moiety of the bispecific or multi-specific antibodies provided herein comprises one or two or three heavy chain complementarity determining regions (HCDR1, HCDR2 and/or HCDR3) contained within any one of the heavy chain variable (VH) region sequences selected from the group consisting of SEQ ID NO: 257, 270 and 313; and/or one or two or three light chain complementarity determining regions (LCDR1, LCDR2 and/or LCDR3) contained with the light chain variable (VL) region sequences selected from the group consisting of SEQ ID NO: 258, 271 and 314. In some embodiments, the CD19 binding moiety of the bispecific or multi-specific antibodies provided herein comprises a homologous sequence thereof having at least 80% (e.g., at least 85%, at least 90%, at least 91%, at least 92%, at least 93%, at least 94%, at least 95%, at least 96%, at least 97%, at least 98%, and at least 99%) sequence identity to SEQ ID NOs: 257, 270 and 313. In some embodiments, the CD19 binding moiety of the bispecific or multi-specific antibodies provided herein comprises a homologous sequence thereof having at least 80% (e.g., at least 85%, at least 90%, at least 91%, at least 92%, at least 93%, at least 94%, at least 95%, at least 96%, at least 97%, at least 98%, and at least 99%) sequence identity to SEQ ID NOs: 258, 271 and 314.

In some embodiments, the CD19 binding moiety of the bispecific or multi-specific antibodies provided herein comprises HCDR1, HCDR2 and/or HCDR3 contained within the VH region sequence of SEQ ID NO: 257, and LCDR1, LCDR2 and/or LCDR3 contained within the VL region sequence of SEQ ID NO: 258.

In some embodiments, the CD19 binding moiety of the bispecific or multi-specific antibodies provided herein comprises HCDR1, HCDR2 and/or HCDR3 contained within the VH region sequence of SEQ ID NO: 270, and LCDR1, LCDR2 and/or LCDR3 contained within the VL region sequence of SEQ ID NO: 271.

In some embodiments, the CD19 binding moiety of the bispecific or multi-specific antibodies provided herein comprises HCDR1, HCDR2 and/or HCDR3 contained within the VH region sequence of SEQ ID NO: 313, and LCDR1, LCDR2 and/or LCDR3 contained within the VL region sequence of SEQ ID NO: 314.

In some embodiments, the CD19 binding moiety of the bispecific or multi-specific antibodies provided herein comprises at least one heavy or light chain complementarity determining region (CDR) comprising an amino acid sequence selected from the group consisting of SEQ ID NOs: 251, 252, 253, 254, 255, 256, 264, 265, 266, 267, 268, 269, 301, 302, 303, 304, 305 and 306.

In some embodiments, the CD19 binding moiety of the bispecific or multi-specific antibodies provided herein comprises one or two or three of HCDR1, HCDR2 and HCDR3 comprising an amino acid sequence selected from the group consisting of SEQ ID NOs: 251, 252, 253, 264, 265, 266, 301, 302 and 303. In some embodiments, the CD19 binding moiety of the bispecific or multi-specific antibodies provided herein comprises one or two or three of LCDR1, LCDR2 and LCDR3 comprising an amino acid sequence selected from the group consisting of SEQ ID NOs: 254, 255, 256, 267, 268, 269, 304, 305 and 306.

In some embodiments, the CD19 binding moiety of the bispecific or multi-specific antibodies provided herein comprises a HCDR1 comprising an amino acid sequence as set forth in SEQ ID NO: 251, 264 or 301, a HCDR2 comprising an amino acid sequence as set forth in SEQ ID NO: 252, 265 or 302, a HCDR3 comprising an amino acid sequence as set forth in SEQ ID NO: 253, 266 or 303.

In some embodiments, the CD19 binding moiety of the bispecific or multi-specific antibodies provided herein comprises a LCDR1 comprising an amino acid sequence as set forth in SEQ ID NO: 254, 267 or 304, a LCDR2 comprising an amino acid sequence as set forth in SEQ ID NO: 255, 268 or 305, a LCDR3 comprising an amino acid sequence as set forth in SEQ ID NO: 256, 269 or 306.

In some embodiments, the CD19 binding moiety of the bispecific or multi-specific antibodies provided herein comprises:
i. a HCDR1 comprising an amino acid sequence as set forth in SEQ ID NO: 251, a HCDR2 comprising an amino acid sequence as set forth in SEQ ID NO: 252, and a HCDR3 comprising an amino acid sequence as set forth in SEQ ID NO: 253;
ii. a HCDR1 comprising an amino acid sequence as set forth in SEQ ID NO: 264, a HCDR2 comprising an amino acid sequence as set forth in SEQ ID NO: 265, and a HCDR3 comprising an amino acid sequence as set forth in SEQ ID NO: 266; or
iii. a HCDR1 comprising an amino acid sequence as set forth in SEQ ID NO: 301, a HCDR2 comprising an amino acid sequence as set forth in SEQ ID NO: 302, and a HCDR3 comprising an amino acid sequence as set forth in SEQ ID NO: 303.

In some embodiments, the CD19 binding moiety of the bispecific or multi-specific antibodies provided herein comprises:
i. a LCDR1 comprising an amino acid sequence as set forth in SEQ ID NO: 254, a LCDR2 comprising an amino acid sequence as set forth in SEQ ID NO: 255, and a LCDR3 comprising an amino acid sequence as set forth in SEQ ID NO: 256;
ii. a LCDR1 comprising an amino acid sequence as set forth in SEQ ID NO: 267, a LCDR2 comprising an amino acid sequence as set forth in SEQ ID NO: 268, and a LCDR3 comprising an amino acid sequence as set forth in SEQ ID NO: 269; or
iii. a LCDR1 comprising an amino acid sequence as set forth in SEQ ID NO: 304, a LCDR2 comprising an amino acid sequence as set forth in SEQ ID NO: 305, and a LCDR3 comprising an amino acid sequence as set forth in SEQ ID NO: 306.

In some embodiments, the CD19 binding moiety of the bispecific or multi-specific antibodies provided herein comprises:
i. a HCDR1 comprising an amino acid sequence as set forth in SEQ ID NO: 251, a HCDR2 comprising an amino acid sequence as set forth in SEQ ID NO: 252, and a HCDR3 comprising an amino acid sequence as set forth in SEQ ID NO: 253, a LCDR1 comprising an amino acid sequence as set forth in SEQ ID NO: 254, a LCDR2 comprising an amino acid sequence as set forth in SEQ ID NO: 255, and a LCDR3 comprising an amino acid sequence as set forth in SEQ ID NO: 256;
ii. a HCDR1 comprising an amino acid sequence as set forth in SEQ ID NO: 264, a HCDR2 comprising an amino acid sequence as set forth in SEQ ID NO: 265, and a HCDR3 comprising an amino acid sequence as set forth in SEQ ID NO: 266; a LCDR1 comprising an amino acid sequence as set forth in SEQ ID NO: 267, a LCDR2 comprising an amino acid sequence as set forth in SEQ ID NO: 268, and a LCDR3 comprising an amino acid sequence as set forth in SEQ ID NO: 269; or
iii. a HCDR1 comprising an amino acid sequence as set forth in SEQ ID NO: 301, a HCDR2 comprising an amino acid sequence as set forth in SEQ ID NO: 302, and a HCDR3 comprising an amino acid sequence as set forth in SEQ ID NO: 303, a LCDR1 comprising an amino acid sequence as set forth in SEQ ID NO: 304, a LCDR2 comprising an amino acid sequence as set forth in SEQ ID NO: 305, and a LCDR3 comprising an amino acid sequence as set forth in SEQ ID NO: 306.

In some embodiments, the CDR boundaries of the CD19 binding moiety are identified by the convention of Kabat. In some embodiments, the CDR boundaries of the CD19 binding moiety are identified by the convention of IMGT. In some embodiments, the CDR boundaries of the CD19 binding moiety are identified by the convention of Chothia. In some embodiments, the CDR boundaries of the CD19 binding moiety are identified by the convention of Al-Lazikani.

The SEQ ID NOs of the heavy chain (denoted as “H”) variable region, light chain (denoted as “L”) variable region, HCDRs and LCDRs of each of the second binding moiety that binds to CD19 described above are shown in Table 50 below. The amino acid sequences of each CDR of the exemplary second binding moieties that binds to CD19 are shown in Table 51 below. Unless otherwise indicated, the CDR boundaries as described in Table 51 below were defined or identified by the convention of Kabat. The amino acid sequences of each VH and VL of the exemplary second binding moieties that binds to CD19 are shown in Table 52 below.

TABLE 50
SEQ ID NOs of VH, VL, HCDRs and LCDRs of Exemplary
Second Binding Moiety that Binds to CD19

| | | Variable | CDR1 | CDR2 | CDR3 |
|---|---|---|---|---|---|
| | | Region | (SEQ | (SEQ | (SEQ |
| | | (SEQ | ID | ID | ID |
| Antibody | Chain | ID NO) | NO) | NO) | NO) |
| C19F | H | 257 | 251 | 252 | 253 |
| | L | 258 | 254 | 255 | 256 |
| C19B | H | 270 | 264 | 265 | 266 |
| | L | 271 | 267 | 268 | 269 |
| mCD19-1D3 | H | 313 | 301 | 302 | 303 |
| | L | 314 | 304 | 305 | 306 |

TABLE 51
Amino Acid Sequences of CDRs of Exemplary Second
Binding Moiety that Binds to CD19

| CDR | SEQ ID NO: | Amino Acid Sequence |
|---|---|---|
| HCDR1 | 251 | DYGVS |
| | 264 | SYWMN |
| | 301 | FYYMH |
| HCDR2 | 252 | VIWGSETTYYNSALKS |
| | 265 | QIWPGDGDTNYNGKFKG |
| | 302 | RIDPEDESTKYSEKFKN |
| HCDR3 | 253 | HYYYGGSYAMDY |
| | 266 | RETTTVGRYYYAMDY |
| | 303 | GGYYFDY |
| LCDR1 | 254 | RASQDISKYLN |
| | 267 | KASQSVDYDGDSYLN |
| | 304 | QASEDIYSGLA |
| LCDR2 | 255 | HTSRLHS |
| | 268 | DASNLVS |
| | 305 | GASDLQD |
| LCDR3 | 256 | QQGNTLPYT |
| | 269 | QQSTEDPWT |
| | 306 | QQGLTYPRT |

TABLE 52
Amino Acid Sequences of Each VH and VL of Exemplary Second
Binding Moiety that Binds to CD19

| Antibody | VH (SEQ ID NO) | VL (SEQ ID NO) |
|---|---|---|
| C19F | EVKLQESGPGLVAPSQSLSVT | DIQMTQTTSSLSASLGDRVT |
| | CTVSGVSLPDYGVSWIRQPPR | ISCRASQDISKYLNWYQQKP |
| | KGLEWLGVIWGSETTYYNSAL | DGTVKLLIYHTSRLHSGVPS |
| | KSRLTIIKDNSKSQVFLKMNSL | RFSGSGSGTDYSLTISNLEQ |
| | QTDDTAIYYCAKHYYYGGSY | EDIATYFCQQGNTLPYTFGG |
| | AMDYWGQGTSVTVSS | GTKLEIT |
| | (SEQ ID NO: 257) | (SEQ ID NO: 258) |
| C19B | QVQLQQSGAELVRPGSSVKIS | DIQLTQSPASLAVSLGQRAT |
| | CKASGYAFSSYWMNWVKQRP | ISCKASQSVDYDGDSYLNW |
| | GQGLEWIGQIWPGDGDTNYN | YQQIPGQPPKLLIYDASNLV |
| | GKFKGKATLTADESSSTAYMQ | SGIPPRFSGSGSGTDFTLNIH |
| | LSSLASEDSAVYFCARRETTTV | PVEKVDAATYHCQQSTEDP |
| | GRYYYAMDYWGQGTTVTVSS | WTFGGGTKLEIK |
| | (SEQ ID NO: 270) | (SEQ ID NO: 271) |
| mCD19- | EVQLQQSGAELVRPGTSVKLS | DIQMTQSPASLSTSLGETVTI |
| 1D3 | CKVSGDTITFYYMHFVKQRPG | QCQASEDIYSGLAWYQQKP |
| | QGLEWIGRIDPEDESTKYSEKF | GKSPQLLIYGASDLQDGVPS |
| | KNKATLTADTSSNTAYLKLSS | RFSGSGSGTQYSLKITSMQT |
| | LTSEDTATYFCIYGGYYFDYW | EDEGVYFCQQGLTYPRTFG |
| | GQGVMVTVSS | GGTKLELK |
| | (SEQ ID NO: 313) | (SEQ ID NO: 314) |

An exemplary bispecific antibody or antigen-binding fragment thereof provided herein comprises:
(1) a CD3 binding moiety comprising HCDR1, HCDR2, and HCDR3 contained within VH region sequence as set forth in SEQ ID NO: 201, and LCDR1, LCDR2, and LCDR3 contained within VL region sequence as set forth in SEQ ID NO: 198; and a CD19 binding moiety comprising HCDR1, HCDR2, and HCDR3 contained within VH region sequence as set forth in SEQ ID NO: 257, and LCDR1, LCDR2, and LCDR3 contained within VL region sequence as set forth in SEQ ID NO: 258;
(2) a CD3 binding moiety comprising HCDR1, HCDR2, and HCDR3 contained within VH region sequence as set forth in SEQ ID NO: 201, and LCDR1, LCDR2, and LCDR3 contained within VL region sequence as set forth in SEQ ID NO: 198; and a CD19 binding moiety comprising HCDR1, HCDR2, and HCDR3 contained within VH region sequence as set forth in SEQ ID NO: 270, and LCDR1, LCDR2, and LCDR3 contained within VL region sequence as set forth in SEQ ID NO: 271;
(3) a CD3 binding moiety comprising HCDR1, HCDR2, and HCDR3 contained within VH region sequence as set forth in SEQ ID NO: 201, and LCDR1, LCDR2, and LCDR3 contained within VL region sequence as set forth in SEQ ID NO: 198; and a CD19 binding moiety comprising HCDR1, HCDR2, and HCDR3 contained within VH region sequence as set forth in SEQ ID NO: 313, and LCDR1, LCDR2, and LCDR3 contained within VL region sequence as set forth in SEQ ID NO: 314;
(4) a CD3 binding moiety comprising HCDR1, HCDR2, and HCDR3 contained within VH region sequence as set forth in SEQ ID NO: 120, and LCDR1, LCDR2, and LCDR3 contained within VL region sequence as set forth in SEQ ID NO: 198; and a CD19 binding moiety comprising HCDR1, HCDR2, and HCDR3 contained within VH region sequence as set forth in SEQ ID NO: 257, and LCDR1, LCDR2, and LCDR3 contained within VL region sequence as set forth in SEQ ID NO: 258;
(5) a CD3 binding moiety comprising HCDR1, HCDR2, and HCDR3 contained within VH region sequence as set forth in SEQ ID NO: 120, and LCDR1, LCDR2, and LCDR3 contained within VL region sequence as set forth in SEQ ID NO: 198; and a CD19 binding moiety comprising HCDR1, HCDR2, NO: 270, and LCDR1, LCDR2, and LCDR3 contained within VL region sequence as set forth in SEQ ID NO: 271;
(6) a CD3 binding moiety comprising HCDR1, HCDR2, and HCDR3 contained within VH region sequence as set forth in SEQ ID NO: 120, and LCDR1, LCDR2, and LCDR3 contained within VL region sequence as set forth in SEQ ID NO: 198; and a CD19 binding moiety comprising HCDR1, HCDR2, and HCDR3 contained within VH region sequence as set forth in SEQ ID NO: 313, and LCDR1, LCDR2, and LCDR3 contained within VL region sequence as set forth in SEQ ID NO: 314;
(7) a CD3 binding moiety comprising HCDR1, HCDR2, and HCDR3 contained within VH region sequence as set forth in SEQ ID NO: 204, and LCDR1, LCDR2, and LCDR3 contained within VL region sequence as set forth in SEQ ID NO: 198; and a CD19 binding moiety comprising HCDR1, HCDR2, and HCDR3 contained within VH region sequence as set forth in SEQ ID NO: 257, and LCDR1, LCDR2, and LCDR3 contained within VL region sequence as set forth in SEQ ID NO: 258;
(8) a CD3 binding moiety comprising HCDR1, HCDR2, and HCDR3 contained within VH region sequence as set forth in SEQ ID NO: 204, and LCDR1, LCDR2, and LCDR3 contained within VL region sequence as set forth in SEQ ID NO: 198; and a CD19 binding moiety comprising HCDR1, HCDR2, and HCDR3 contained within VH region sequence as set forth in SEQ ID NO: 270, and LCDR1, LCDR2, and LCDR3 contained within VL region sequence as set forth in SEQ ID NO: 271;
(9) a CD3 binding moiety comprising HCDR1, HCDR2, and HCDR3 contained within VH region sequence as set forth in SEQ ID NO: 204, and LCDR1, LCDR2, and LCDR3 contained within VL region sequence as set forth in SEQ ID NO: 198; and a CD19 binding moiety comprising HCDR1, HCDR2, NO: 313, and LCDR1, LCDR2, and LCDR3 contained within VL region sequence as set forth in SEQ ID NO: 314;
(10) a CD3 binding moiety comprising HCDR1, HCDR2, and HCDR3 contained within VH region sequence as set forth in SEQ ID NO: 315, and LCDR1, LCDR2, and LCDR3 contained within VL region sequence as set forth in SEQ ID NO: 316; and a CD19 binding moiety comprising HCDR1, HCDR2, and HCDR3 contained within VH region sequence as set forth in SEQ ID NO: 257, and LCDR1, LCDR2, and LCDR3 contained within VL region sequence as set forth in SEQ ID NO: 258;
(11) a CD3 binding moiety comprising HCDR1, HCDR2, and HCDR3 contained within VH region sequence as set forth in SEQ ID NO: 315, and LCDR1, LCDR2, and LCDR3 contained within VL region sequence as set forth in SEQ ID NO: 316; and a CD19 binding moiety comprising HCDR1, HCDR2, and HCDR3 contained within VH region sequence as set forth in SEQ ID NO: 270, and LCDR1, LCDR2, and LCDR3 contained within VL region sequence as set forth in SEQ ID NO: 271;
(12) a CD3 binding moiety comprising HCDR1, HCDR2, and HCDR3 contained within VH region sequence as set forth in SEQ ID NO: 315, and LCDR1, LCDR2, and LCDR3 contained within VL region sequence as set forth in SEQ ID NO: 316; and a CD19 binding moiety comprising HCDR1, HCDR2, and HCDR3 contained within VH region sequence as set forth in SEQ ID NO: 313, and LCDR1, LCDR2, and LCDR3 contained within VL region sequence as set forth in SEQ ID NO: 314.

In certain embodiments, the bispecific or multi-specific antibody or antigen-binding fragment thereof provided herein can also comprise a first binding moiety that binds to CD3 (as referred to as “CD3 binding moiety” in the present disclosure) provided herein, and a second binding moiety that binds to CD20 (also referred to as “CD20 binding moiety” in the present disclosure), wherein the first binding moiety and the second binding moiety are independently a single-chain Fv (scFv) or a Fab domain.

In certain embodiments, the bispecific or multi-specific antibodies or antigen-binding fragments thereof provided herein bind to CD3 and CD20. In some embodiments, the bispecific or multi-specific antibodies or antigen-binding fragments thereof provided herein are in a Fab-scFv-Fab format. In certain embodiments, the bispecific or multi-specific antibodies or antigen-binding fragments thereof provided herein comprise two anti-CD20 Fab domains, and one anti-CD3 scFv domain. In certain embodiments, the bispecific or multi-specific antibodies or antigen-binding fragments thereof provided herein comprise two anti-CD3 Fab domains, and one anti-CD20 scFv domain.

As used herein, the term “CD20 binding moiety” when referring to a bispecific or multi-specific antibody or antigen-binding fragment thereof, refers to a moiety that is capable of binding to CD20 (e.g., human CD20, mouse CD20, or cynomolgus CD20). The CD20 binding moiety can take any form that allows specific recognition of the target CD20. For example, the CD20 binding moiety may be an antibody or an antigen-binding fragment thereof, e.g., an IgG (such as IgG1, IgG2, IgG3, and IgG4) antibody, IgA antibody, or IgM antibody. In some embodiments, the CD20 binding moiety of the bispecific or multi-specific antibodies provided herein is derived from an anti-CD20 antibody. The CD20 binding moiety of the bispecific or multi-specific antibodies provided herein may be derived from any of the anti-CD20 antibodies known in the art, e.g., the anti-CD20 antibodies described in WO2011100403A1, WO2008063771A2, etc.

In some embodiments, the CD20 binding moiety of the bispecific or multi-specific antibodies provided herein comprises one or two or three heavy chain complementarity determining regions (HCDR1, HCDR2 and/or HCDR3) contained within any one of the heavy chain variable (VH) region sequences selected from the group consisting of SEQ ID NO: 282, 294 and 326; and/or one or two or three light chain complementarity determining regions (LCDR1, LCDR2 and/or LCDR3) contained with the light chain variable (VL) region sequences selected from the group consisting of SEQ ID NO: 283, 295 and 327. In some embodiments, the CD20 binding moiety of the bispecific or multi-specific antibodies provided herein comprises a homologous sequence thereof having at least 80% (e.g., at least 85%, at least 90%, at least 91%, at least 92%, at least 93%, at least 94%, at least 95%, at least 96%, at least 97%, at least 98%, and at least 99%) sequence identity to SEQ ID NOs: 282, 294 and 326. In some embodiments, the CD20 binding moiety of the bispecific or multi-specific antibodies provided herein comprises a homologous sequence thereof having at least 80% (e.g., at least 85%, at least 90%, at least 91%, at least 92%, at least 93%, at least 94%, at least 95%, at least 96%, at least 97%, at least 98%, and at least 99%) sequence identity to SEQ ID NOs: 283, 295 and 327.

In some embodiments, the CD20 binding moiety of the bispecific or multi-specific antibodies provided herein comprises HCDR1, HCDR2 and/or HCDR3 contained within the VH region sequence of SEQ ID NO: 282, and LCDR1, LCDR2 and/or LCDR3 contained within the VL region sequence of SEQ ID NO: 283.

In some embodiments, the CD20 binding moiety of the bispecific or multi-specific antibodies provided herein comprises HCDR1, HCDR2 and/or HCDR3 contained within the VH region sequence of SEQ ID NO: 294, and LCDR1, LCDR2 and/or LCDR3 contained within the VL region sequence of SEQ ID NO: 295.

In some embodiments, the CD20 binding moiety of the bispecific or multi-specific antibodies provided herein comprises HCDR1, HCDR2 and/or HCDR3 contained within the VH region sequence of SEQ ID NO: 326, and LCDR1, LCDR2 and/or LCDR3 contained within the VL region sequence of SEQ ID NO: 327.

In some embodiments, the CD20 binding moiety of the bispecific or multi-specific antibodies provided herein comprises at least one heavy or light chain complementarity determining region (CDR) comprising an amino acid sequence selected from the group consisting of SEQ ID NOs: 276, 277, 278, 279, 280, 281, 288, 289, 290, 291, 292, 293, 320, 321, 322, 323, 324 and 325.

In some embodiments, the CD20 binding moiety of the bispecific or multi-specific antibodies provided herein comprises one or two or three of HCDR1, HCDR2 and HCDR3 comprising an amino acid sequence selected from the group consisting of SEQ ID NOs: 276, 277, 278, 288, 289, 290, 320, 321 and 322. In some embodiments, the CD20 binding moiety of the bispecific or multi-specific antibodies provided herein comprises one or two or three of LCDR1, LCDR2 and LCDR3 comprising an amino acid sequence selected from the group consisting of SEQ ID NOs: 279, 280, 281, 291, 292, 293, 323, 324 and 325.

In some embodiments, the CD20 binding moiety of the bispecific or multi-specific antibodies provided herein comprises a HCDR1 comprising an amino acid sequence as set forth in SEQ ID NO: 276, 288 or 320, a HCDR2 comprising an amino acid sequence as set forth in SEQ ID NO: 277, 289 or 321, a HCDR3 comprising an amino acid sequence as set forth in SEQ ID NO: 278, 290 or 322.

In some embodiments, the CD20 binding moiety of the bispecific or multi-specific antibodies provided herein comprises a LCDR1 comprising an amino acid sequence as set forth in SEQ ID NO: 279, 291 or 323, a LCDR2 comprising an amino acid sequence as set forth in SEQ ID NO: 280, 292 or 324, a LCDR3 comprising an amino acid sequence as set forth in SEQ ID NO: 281, 293 or 325.

In some embodiments, the CD20 binding moiety of the bispecific or multi-specific antibodies provided herein comprises:
i. a HCDR1 comprising an amino acid sequence as set forth in SEQ ID NO: 276, a HCDR2 comprising an amino acid sequence as set forth in SEQ ID NO: 277, and a HCDR3 comprising an amino acid sequence as set forth in SEQ ID NO: 278;
ii. a HCDR1 comprising an amino acid sequence as set forth in SEQ ID NO: 288, a HCDR2 comprising an amino acid sequence as set forth in SEQ ID NO: 289, and a HCDR3 comprising an amino acid sequence as set forth in SEQ ID NO: 290; or
iii. a HCDR1 comprising an amino acid sequence as set forth in SEQ ID NO: 320, a HCDR2 comprising an amino acid sequence as set forth in SEQ ID NO: 321, and a HCDR3 comprising an amino acid sequence as set forth in SEQ ID NO: 322.

In some embodiments, the CD20 binding moiety of the bispecific or multi-specific antibodies provided herein comprises:
i. a LCDR1 comprising an amino acid sequence as set forth in SEQ ID NO: 279, a LCDR2 comprising an amino acid sequence as set forth in SEQ ID NO: 280, and a LCDR3 comprising an amino acid sequence as set forth in SEQ ID NO: 281;
ii. a LCDR1 comprising an amino acid sequence as set forth in SEQ ID NO: 291, a LCDR2 comprising an amino acid sequence as set forth in SEQ ID NO: 292, and a LCDR3 comprising an amino acid sequence as set forth in SEQ ID NO: 293; or
iii. a LCDR1 comprising an amino acid sequence as set forth in SEQ ID NO: 323, a LCDR2 comprising an amino acid sequence as set forth in SEQ ID NO: 324, and a LCDR3 comprising an amino acid sequence as set forth in SEQ ID NO: 325.

In some embodiments, the CD20 binding moiety of the bispecific or multi-specific antibodies provided herein comprises:
i. a HCDR1 comprising an amino acid sequence as set forth in SEQ ID NO: 276, a HCDR2 comprising an amino acid sequence as set forth in SEQ ID NO: 277, and a HCDR3 comprising an amino acid sequence as set forth in SEQ ID NO: 278, a LCDR1 comprising an amino acid sequence as set forth in SEQ ID NO: 279, a LCDR2 comprising an amino acid sequence as set forth in SEQ ID NO: 280, and a LCDR3 comprising an amino acid sequence as set forth in SEQ ID NO: 281;
ii. a HCDR1 comprising an amino acid sequence as set forth in SEQ ID NO: 288, a HCDR2 comprising an amino acid sequence as set forth in SEQ ID NO: 289, and a HCDR3 comprising an amino acid sequence as set forth in SEQ ID NO: 290, a LCDR1 comprising an amino acid sequence as set forth in SEQ ID NO: 291, a LCDR2 comprising an amino acid sequence as set forth in SEQ ID NO: 292, and a LCDR3 comprising an amino acid sequence as set forth in SEQ ID NO: 293; or
iii. a HCDR1 comprising an amino acid sequence as set forth in SEQ ID NO: 320, a HCDR2 comprising an amino acid sequence as set forth in SEQ ID NO: 321, and a HCDR3 comprising an amino acid sequence as set forth in SEQ ID NO: 322, a LCDR1 comprising an amino acid sequence as set forth in SEQ ID NO: 323, a LCDR2 comprising an amino acid sequence as set forth in SEQ ID NO: 324, and a LCDR3 comprising an amino acid sequence as set forth in SEQ ID NO: 325.

In some embodiments, the CDR boundaries of the CD20 binding moiety are identified by the convention of Kabat. In some embodiments, the CDR boundaries of the CD20 binding moiety are identified by the convention of IMGT. In some embodiments, the CDR boundaries of the CD20 binding moiety are identified by the convention of Chothia. In some embodiments, the CDR boundaries of the CD20 binding moiety are identified by the convention of Al-Lazikani.

The SEQ ID NOs of the heavy chain (denoted as “H”) variable region, light chain (denoted as “L”) variable region, HCDRs and LCDRs of each of the second binding moiety that binds to CD20 described above are shown in Table 53 below. The amino acid sequences of each CDR of the exemplary second binding moieties that binds to CD20 are shown in Table 54 below. Unless otherwise indicated, the CDR boundaries as described in Table 54 below were defined or identified by the convention of Kabat. The amino acid sequences of each VH and VL of the exemplary second binding moieties that binds to CD20 are shown in Table 55 below.

TABLE 53
SEQ ID NOs of VH, VL, HCDRs and LCDRs of Exemplary
Second Binding Moiety that Binds to CD20

| | | Variable | CDR1 | CDR2 | CDR3 |
|---|---|---|---|---|---|
| | | Region | (SEQ | (SEQ | (SEQ |
| | | (SEQ | ID | ID | ID |
| Antibody | Chain | ID NO) | NO) | NO) | NO) |
| C20O | H | 282 | 276 | 277 | 278 |
| | L | 283 | 279 | 280 | 281 |
| C20R | H | 294 | 288 | 289 | 290 |
| | L | 295 | 291 | 292 | 293 |
| mCD20-18B12 | H | 326 | 320 | 321 | 322 |
| | L | 327 | 323 | 324 | 325 |

TABLE 54
Amino Acid Sequences of CDRs of Exemplary Second
Binding Moiety that Binds to CD20

| CDR | SEQ ID NO: | Amino Acid Sequence |
|---|---|---|
| HCDR1 | 276 | DYAMH |
| | 288 | SYNMH |
| | 320 | SYWMH |
| HCDR2 | 277 | TISWNSGSIGYADSVKG |
| | 289 | AIYPGNGDTSYNQKFKG |
| | 321 | VIDPSDNYTKYNQKFKG |
| HCDR3 | 278 | DIQYGNYYYGMDV |
| | 290 | STYYGGDWYFNV |
| | 322 | EGYYGSSPWFAY |
| LCDR1 | 279 | RASQSVSSYLA |
| | 291 | RASSSVSYIH |
| | 323 | RARSSVSYIH |
| LCDR2 | 280 | DASNRAT |
| | 292 | ATSNLAS |
| | 324 | ATSNLAS |
| LCDR3 | 281 | QQRSNWPIT |
| | 293 | QQWTSNPPT |
| | 325 | QQWSSKPPT |

TABLE 55
Amino Acid Sequences of Each VH and VL of Exemplary Second
Binding Moiety that Binds to CD20

| Antibody | VH (SEQ ID NO) | VL (SEQ ID NO) |
|---|---|---|
| C200 | EVQLVESGGGLVQPGRSLRLS | EIVLTQSPATLSLSPGERATL |
| | CAASGFTFNDYAMHWVRQAP | SCRASQSVSSYLAWYQQKP |
| | GKGLEWVSTISWNSGSIGYAD | GQAPRLLIYDASNRATGIPA |
| | SVKGRFTISRDNAKKSLYLQM | RFSGSGSGTDFTLTISSLEPE |
| | NSLRAEDTALYYCAKDIQYGN | DFAVYYCQQRSNWPITFGQ |
| | YYYGMDVWGQGTTVTVSS | GTRLEIK |
| | (SEQ ID NO: 282) | (SEQ ID NO: 283) |
| C20R | QVQLQQPGAELVKPGASVKM | QIVLSQSPAILSASPGEKVT |
| | SCKASGYTFTSYNMHWVKQT | MTCRASSSVSYIHWFQQKP |
| | PGRGLEWIGAIYPGNGDTSYN | GSSPKPWIYATSNLASGVPV |
| | QKFKGKATLTADKSSSTAYM | RFSGSGSGTSYSLTISRVEAE |
| | QLSSLTSEDSAVYYCARSTYY | DAATYYCQQWTSNPPTFGG |
| | GGDWYFNVWGAGTTVTVSA | GTKLEIK |
| | (SEQ ID NO: 294) | (SEQ ID NO: 295) |
| mCD20- | QVQLQQPGAELVRPGTSVKLS | QIVMSQSPAILSASPGEKVT |
| 18B12 | CKASGYTFTSYWMHWIKQRP | MTCRARSSVSYIHWYQQKP |
| | GQGLEWIGVIDPSDNYTKYNQ | GSSPKPWIYATSNLASGVPG |
| | KFKGKATLTVDTSSSTAYMQL | RFSGSGSGTSYSLTITRVEAE |
| | SSLTSEDSAVYFCAREGYYGSS | DAATYYCQQWSSKPPTFGG |
| | PWFAYWGQGTLVTVSS | GTKLEIK |
| | (SEQ ID NO: 326) | (SEQ ID NO: 327) |

An exemplary bispecific antibody or antigen-binding fragment thereof provided herein comprises:
(1) a CD3 binding moiety comprising HCDR1, HCDR2, and HCDR3 contained within VH region sequence as set forth in SEQ ID NO: 201, and LCDR1, LCDR2, and LCDR3 contained within VL region sequence as set forth in SEQ ID NO: 198; and a CD20 binding moiety comprising HCDR1, HCDR2, and HCDR3 contained within VH region sequence as set forth in SEQ ID NO: 282, and LCDR1, LCDR2, and LCDR3 contained within VL region sequence as set forth in SEQ ID NO: 283;
(2) a CD3 binding moiety comprising HCDR1, HCDR2, and HCDR3 contained within VH region sequence as set forth in SEQ ID NO: 201, and LCDR1, LCDR2, and LCDR3 contained within VL region sequence as set forth in SEQ ID NO: 198; and a CD20 binding moiety comprising HCDR1, HCDR2, and HCDR3 contained within VH region sequence as set forth in SEQ ID NO: 294, and LCDR1, LCDR2, and LCDR3 contained within VL region sequence as set forth in SEQ ID NO: 295;
(3) a CD3 binding moiety comprising HCDR1, HCDR2, and HCDR3 contained within VH region sequence as set forth in SEQ ID NO: 201, and LCDR1, LCDR2, and LCDR3 contained within VL region sequence as set forth in SEQ ID NO: 198; and a CD20 binding moiety comprising HCDR1, HCDR2, and HCDR3 contained within VH region sequence as set forth in SEQ ID NO: 326, and LCDR1, LCDR2, and LCDR3 contained within VL region sequence as set forth in SEQ ID NO: 327;
(4) a CD3 binding moiety comprising HCDR1, HCDR2, and HCDR3 contained within VH region sequence as set forth in SEQ ID NO: 120, and LCDR1, LCDR2, and LCDR3 contained within VL region sequence as set forth in SEQ ID NO: 198; and a CD20 binding moiety comprising HCDR1, HCDR2, and HCDR3 contained within VH region sequence as set forth in SEQ ID NO: 282, and LCDR1, LCDR2, and LCDR3 contained within VL region sequence as set forth in SEQ ID NO: 283;
(5) a CD3 binding moiety comprising HCDR1, HCDR2, and HCDR3 contained within VH region sequence as set forth in SEQ ID NO: 120, and LCDR1, LCDR2, and LCDR3 contained within VL region sequence as set forth in SEQ ID NO: 198; and a CD20 binding moiety comprising HCDR1, HCDR2, and HCDR3 contained within VH region sequence as set forth in SEQ ID NO: 294, and LCDR1, LCDR2, and LCDR3 contained within VL region sequence as set forth in SEQ ID NO: 295;
(6) a CD3 binding moiety comprising HCDR1, HCDR2, and HCDR3 contained within VH region sequence as set forth in SEQ ID NO: 120, and LCDR1, LCDR2, and LCDR3 contained within VL region sequence as set forth in SEQ ID NO: 198; and a CD20 binding moiety comprising HCDR1, HCDR2, and HCDR3 contained within VH region sequence as set forth in SEQ ID NO: 326, and LCDR1, LCDR2, and LCDR3 contained within VL region sequence as set forth in SEQ ID NO: 327;
(7) a CD3 binding moiety comprising HCDR1, HCDR2, and HCDR3 contained within VH region sequence as set forth in SEQ ID NO: 204, and LCDR1, LCDR2, and LCDR3 contained within VL region sequence as set forth in SEQ ID NO: 198; and a CD20 binding moiety comprising HCDR1, HCDR2, and HCDR3 contained within VH region sequence as set forth in SEQ ID NO: 282, and LCDR1, LCDR2, and LCDR3 contained within VL region sequence as set forth in SEQ ID NO: 283;
(8) a CD3 binding moiety comprising HCDR1, HCDR2, and HCDR3 contained within VH region sequence as set forth in SEQ ID NO: 204, and LCDR1, LCDR2, and LCDR3 contained within VL region sequence as set forth in SEQ ID NO: 198; and a CD20 binding moiety comprising HCDR1, HCDR2, and HCDR3 contained within VH region sequence as set forth in SEQ ID NO: 294, and LCDR1, LCDR2, and LCDR3 contained within VL region sequence as set forth in SEQ ID NO: 295;
(9) a CD3 binding moiety comprising HCDR1, HCDR2, and HCDR3 contained within VH region sequence as set forth in SEQ ID NO: 204, and LCDR1, LCDR2, and LCDR3 contained within VL region sequence as set forth in SEQ ID NO: 198; and a CD20 binding moiety comprising HCDR1, HCDR2, and HCDR3 contained within VH region sequence as set forth in SEQ ID NO: 326, and LCDR1, LCDR2, and LCDR3 contained within VL region sequence as set forth in SEQ ID NO: 327;
(10) a CD3 binding moiety comprising HCDR1, HCDR2, and HCDR3 contained within VH region sequence as set forth in SEQ ID NO: 315, and LCDR1, LCDR2, and LCDR3 contained within VL region sequence as set forth in SEQ ID NO: 316; and a CD20 binding moiety comprising HCDR1, HCDR2, and HCDR3 contained within VH region sequence as set forth in SEQ ID NO: 282, and LCDR1, LCDR2, and LCDR3 contained within VL region sequence as set forth in SEQ ID NO: 283;
(11) a CD3 binding moiety comprising HCDR1, HCDR2, and HCDR3 contained within VH region sequence as set forth in SEQ ID NO: 315, and LCDR1, LCDR2, and LCDR3 contained within VL region sequence as set forth in SEQ ID NO: 316; and a CD20 binding moiety comprising HCDR1, HCDR2, and HCDR3 contained within VH region sequence as set forth in SEQ ID NO: 294, and LCDR1, LCDR2, and LCDR3 contained within VL region sequence as set forth in SEQ ID NO: 295;
(12) a CD3 binding moiety comprising HCDR1, HCDR2, and HCDR3 contained within VH region sequence as set forth in SEQ ID NO: 315, and LCDR1, LCDR2, and LCDR3 contained within VL region sequence as set forth in SEQ ID NO: 316; and a CD20 binding moiety comprising HCDR1, HCDR2, and HCDR3 contained within VH region sequence as set forth in SEQ ID NO: 326, and LCDR1, LCDR2, and LCDR3 contained within VL region sequence as set forth in SEQ ID NO: 327.

Different formats of bispecific or multi-specific antibodies have been described in the art, for example, in Chames and Baty (2009) Curr Opin Drug Disc Dev 12:276. The bispecific or multi-specific antibodies or antigen-binding fragments thereof provided herein are not limited to any particular bispecific format, and may have all the different formats known in the art.

For example, bispecific antibodies or antigen-binding fragments thereof provided herein may have a typical full-length antibody structure, i.e., an antibody having two different full-length antibody heavy chains and two different full length antibody light chains. A full-length antibody heavy chain includes heavy chain variable region (VH) and constant domains CH1, CH2, CH3 and optionally CH4. A full-length antibody light chain includes light chain variable region (VL) and constant domain CL. For example, the bispecific antibodies or antigen-binding fragments thereof provided herein may comprises a first light chain, a first heavy chain, a second heavy chain, and a second light chain, wherein the first light chain and the first heavy chain are paired to form a first antigen-binding site that binds to CD3, and the second light chain and the second heavy chain are paired to form a second antigen-binding site that binds to an antigen other than CD3 (e.g., GPRC5D or GD2). For another example, the bispecific antibodies or antigen-binding fragments thereof provided herein may comprises a first light chain, a first heavy chain, a second heavy chain, and a second light chain, wherein the first light chain and the first heavy chain are paired to form a first antigen-binding site that binds to an antigen other than CD3 (e.g., GPRC5D or GD2), and the second light chain and the second heavy chain are paired to form a second antigen-binding site that binds to CD3.

For another example, the bispecific or multi-specific antibodies or antigen-binding fragments thereof provided herein may include, but are not limited to, bispecific antibodies with complementary CH3 domains to force heterodimerization, Knobs-into-Holes molecules (Genentech, WO9850431), CrossMAbs (Roche, WO2009080253), or electrostatically-matched molecules (Amgen, EP1870459 and WO2009089004; Chugai, US201000155133; Oncomed, WO2010129304). For example, regarding the CrossMab format, the GPRC5D binding moiety of the bispecific or multi-specific antibodies provided herein further comprises a constant domain CL and a constant domain CH1, and in some embodiments, the constant domains CL and CH1 are replaced by each other; or the CD3 binding moiety of the bispecific or multi-specific antibodies provided herein further comprises a constant domain CL and a constant domain CH1, and in some embodiments, the constant domains CL and CH1 are replaced by each other.

### Third Binding Moiety

In some embodiments, the bispecific or multi-specific antibody or antigen-binding fragment thereof provided herein comprise a first specificity for CD3, a second specificity and a third specificity. In some embodiments, the third specificity is for CD3 but to different epitopes. In some embodiments, the second specificity is for a second antigen different from CD3.

In certain embodiments, the third specificity is for a tumor associated antigen or an epitope thereof. The term “tumor associated antigen” refers to an antigen that is or can be presented on a tumor cell surface and that is located on or within tumor cells. In some embodiments, the tumor associated antigens can be presented only by tumor cells and not by normal cells, i.e., non-tumor cells. In some other embodiments, the tumor associated antigens can be exclusively expressed on tumor cells or may represent a tumor specific mutation compared to non-tumor cells. In some other embodiments, the tumor associated antigens can be found in both tumor cells and non-tumor cells, but are overexpressed on tumor cells when compared to non-tumor cells or are accessible for antibody binding in tumor cells due to the less compact structure of the tumor tissue compared to non-tumor tissue. In some embodiments, the tumor associated antigen is located on the vasculature of a tumor.

In certain embodiments, the bispecific or multi-specific antibodies or antigen-binding fragments thereof provided herein are capable of binding to one or more (e.g., 1, 2, 3, 4, 5 or more) additional antigens other than CD3. In certain embodiments, the one or more additional antigens other than CD3 are selected from the group consisting of GPRC5D, GD2, CD16a, CD33, CD3ε, CD45, CD123, CD146, CD228, CLL-1, FLT3, FLT3L, TAF1, TgPRF, HVCN1, IL-6R, IL-11R, IL17A, IL-23R, IL-33, ILDR2, LAP, TSLP, TREM-1, ANGPT2, APOE, IFNAR, CypA, DOG-1, NKp30, CSF-1R, CCR2, LRRC15, mesothelin, Dickkopf2, DLL3, HER-2, C10orf54, TrkA, MEKK1, KRAS, ERK, XPO1, mTORC1/2, PAK4, NAMPT, ATR, EGFR, FGFR, VEGF, LILRB (e.g., LILRB1, LILRB2, LILRB3, LILRB4, LILRB5), c-MET, Her2, Her3, CTLA4, GITA, CD112R, CD2, CD7, CD16, CD19, CD20, CD24, CD27, CD30, CD34, CD37, CD39, CD70, CD73, CD83, CD28, CD80 (B7-1), CD86 (B7-2), CD40, CD40L (CD154), CD47, SIRPa, CD122, CD137, CD137L, OX40 (CD134), OX40L (CD252), BCMA (e.g., BCMA02), PSMA, CLDN18 (e.g., CLDN18.2), NKG2C, 4-1BB, LIGHT, PVRIG, SLAMF7, HVEM, BAFFR, ICAM-1, 2B4, LFA-1, GITR, ICOS (CD278), ICOSLG (CD275), LAG3 (CD223), A2AR, B7-H3 (CD276), B7-H4 (VTCN1), B7-H5, BTLA (CD272), CD160, CTLA-4 (CD152), IDO (e.g., IDO1, IDO2), ILT3, TDO, KIR, LAIR-1, NOX2, PD-1, PD-L1, PD-L2, TIM-3, VISTA, SIGLEC-7 (CD328), SIGLEC-9 (CD329), SIGLEC-15, TIGIT, PVR (CD155), TLR3, CLEC9A, DEC-205, STING, and TGFB.

In certain embodiments, the bispecific or multi-specific antibodies or antigen-binding fragments thereof provided herein bind to CD3 and GPRC5D. In certain embodiments, the bispecific or multi-specific antibodies or antigen-binding fragments thereof provided herein bind to CD3 and GD2. In certain embodiments, the bispecific or multi-specific antibodies or antigen-binding fragments thereof provided herein bind to CD3 and LILRB4. In certain embodiments, the bispecific or multi-specific antibodies or antigen-binding fragments thereof provided herein bind to CD3 and CD19. In certain embodiments, the bispecific or multi-specific antibodies or antigen-binding fragments thereof provided herein bind to CD3 and CD20. In certain embodiments, the second target is the same as the third target. In certain embodiments, the second target and third target are both GPRC5D. In certain embodiments, the second target and third target are both GD2. In certain embodiments, the second target and third target are both LILRB4. In certain embodiments, the second target and third target are both CD19. In certain embodiments, the second target and third target are both CD20. In certain embodiments, the sequence of the second binding moiety is the same as the sequence of the third binding moiety.

FR region

In some embodiments, the bispecific or multi-specific antibodies or antigen-binding fragments thereof provided herein are humanized. In certain embodiments, the humanized antibodies or antigen-binding fragments thereof provided herein are composed of substantially all human sequences except for the CDR sequences which are non-human. In some embodiments, the variable region FRs, and constant regions if present, are entirely or substantially from human immunoglobulin sequences. The human FR sequences and human constant region sequences may be derived from different human immunoglobulin genes, for example, FR sequences derived from one human antibody and constant region from another human antibody. In some embodiments, the humanized antibody or antigen-binding fragment thereof comprises human heavy chain HFR1, HFR2, HFR3 and HFR4, and/or light chain LFR1, LFR2, LFR3 and LFR4.

In some embodiments, the FR regions derived from human may comprise the same amino acid sequence as the human immunoglobulin from which it is derived. In some embodiments, one or more amino acid residues of the human FR are substituted with the corresponding residues from the parent non-human antibody. This may be desirable in certain embodiments to make the humanized antibody or its fragment closely approximate the non-human parent antibody structure, so as to optimize binding characteristics (for example, increase binding affinity). In certain embodiments, the humanized antibody or antigen-binding fragment thereof provided herein comprises no more than 10, 9, 8, 7, 6, 5, 4, 3, 2, or 1 amino acid residue substitutions in each of the human FR sequences, or no more than 10, 9, 8, 7, 6, 5, 4, 3, 2, or 1 amino acid residue substitutions in all the FR sequences of a heavy or a light chain variable domain. In some embodiments, such change in amino acid residue could be present in heavy chain FR regions only, in light chain FR regions only, or in both chains. In certain embodiments, one or more amino acids of the human FR sequences are randomly mutated to increase binding affinity. In certain embodiments, one or more amino acids of the human FR sequences are back mutated to the corresponding amino acid(s) of the parent non-human antibody so as to increase binding affinity. In certain embodiments, one or more amino acids of the human FR sequences of VH and VL regions are mutated to form a disulfide bond between the VH and VL regions (e.g., between the VH and VL regions of the first binding moiety). Without being bound by any theory, the disulfide bond is helpful for preventing aggregation of antibodies, and thus can facilitate stability of antibodies. In some embodiments, a disulfide bond is formed between VH and VL regions of the first binding moiety by G44C (Kabat numbering) mutation in FR of VH region, and G100C (Kabat numbering) mutation in FR of VL region.

In some embodiments, the bispecific or multi-specific antibodies and antigen-binding fragments thereof provided herein comprise all or a portion of the heavy chain variable domain and/or all or a portion of the light chain variable domain. In one embodiment, the bispecific or multi-specific antibody or antigen-binding fragment thereof provided herein is a single domain antibody which consists of all or a portion of the heavy chain variable domain provided herein. More information of such a single domain antibody is available in the art (see, e.g., U.S. Pat. No. 6,248,516).

### Fc Region

In certain embodiments, the bispecific or multi-specific antibodies or antigen-binding fragments thereof provided herein further comprise an Fc region. In certain embodiments, the bispecific or multi-specific antibodies or antigen-binding fragments thereof provided herein further comprise an Fc region, optionally an Fc region of human immunoglobulin (Ig), or optionally an Fc region of human IgG. In certain embodiments, the bispecific or multi-specific antibodies or antigen-binding fragments thereof provided herein further comprise a constant region, which optionally further comprises a heavy chain and/or a light chain constant region. In certain embodiments, the heavy chain constant region comprises CH1, hinge, and/or CH2-CH3 regions (or optionally CH2-CH3-CH4 regions). In certain embodiments, the bispecific or multi-specific antibodies or antigen-binding fragments thereof provided herein comprise heavy chain constant regions of human IgG1, IgG2, IgG3 or IgG4. In certain embodiments, the first binding moiety and the third binding moiety are linked to the Fc region. In certain embodiments, the bispecific or multi-specific antibodies or antigen-binding fragments thereof provided herein comprise a lambda (λ) light chain or a kappa (κ) light chain. The constant region of the bispecific or multi-specific antibodies or antigen-binding fragments thereof provided herein may be identical to the wild-type constant region sequence or be different in one or more mutations.

In certain embodiments, the heavy chain constant region comprises an Fc region. Fc region is known to mediate effector functions such as antibody-dependent cellular cytotoxicity (ADCC) and complement-dependent cytotoxicity (CDC) of the antibody. Fc regions of different Ig isotypes have different abilities to induce effector functions. For example, Fc regions of IgG1 and IgG3 have been recognized to induce both ADCC and CDC more effectively than those of IgG2 and IgG4. In certain embodiments, the bispecific or multi-specific antibodies and antigen-binding fragments thereof provided herein comprises an Fc region of IgG1, or IgG3 isotype, which could induce ADCC or CDC; or alternatively, a constant region of IgG4 or IgG2 isotype, which has reduced or depleted effector function. In some embodiments, the Fc region derived from human IgG1 with enhanced effector functions.

### Competing for Binding to Epitopes

In certain embodiments, the antibodies or antigen-binding fragments thereof provided herein have a specific binding affinity to CD3 and/or targets other than CD3 which is sufficient to provide for diagnostic and/or therapeutic use.

The antibodies or antigen-binding fragments thereof provided herein can be a monoclonal antibody, a polyclonal antibody, a humanized antibody, a human antibody, a chimeric antibody, a recombinant antibody, a bispecific antibody, a multi-specific antibody, a labeled antibody, a bivalent antibody, an anti-idiotypic antibody, or a fusion protein. A recombinant antibody is an antibody prepared in vitro using recombinant methods rather than in animals.

In certain embodiments, the present disclosure provides a bispecific or multi-specific antibody or antigen-binding fragment thereof, which competes for binding to CD3 and/or targets other than CD3 with the antibody or antigen-binding fragment thereof provided herein. In certain embodiments, the present disclosure provides a bispecific or multi-specific antibody or antigen-binding fragment thereof, which competes for binding to human CD3 and/or targets other than CD3 with any one of the anti-CD3 antibodies provided herein. In some embodiments, the present disclosure provides a bispecific or multi-specific antibody or antigen-binding fragment thereof, which competes for the same epitope with the antibody or antigen-binding fragment thereof provided herein.

The ability to “block binding” or “compete for the same epitope” as used herein refers to the ability of an antibody or antigen-binding fragment to inhibit the binding interaction between two molecules (e.g., human CD3 and an anti-CD3 antibody) to any detectable degree. In certain embodiments, an antibody or antigen-binding fragment that blocks binding between two molecules inhibits the binding interaction between the two molecules by at least 85%, or at least 90%. In certain embodiments, this inhibition may be greater than 85%, or greater than 90%.

Those skilled in the art will recognize that it is possible to determine, without undue experimentation, if a human monoclonal antibody binds to the same epitope as the antibody of present disclosure by ascertaining whether the former prevents the latter from binding to a CD3 antigen polypeptide and/or targets other than CD3 antigen polypeptide. If the test antibody competes with the antibody of the present disclosure, as shown by a decrease in binding by the antibody of present disclosure to the CD3 antigen polypeptide and/or targets other than CD3 antigen polypeptide, then the two antibodies bind to the same, or a closely related, epitope. Or if the binding of a test antibody to the CD3 antigen polypeptide and/or targets other than CD3 antigen polypeptide was inhibited by the antibody of the present disclosure, then the two antibodies bind to the same, or a closely related, epitope.

In certain embodiments, the present disclosure provides bispecific or multi-specific antibodies or antigen-binding fragments thereof which have higher or comparable binding affinity to CD3 (e.g., human CD3 or cynomolgus CD3) and GPRC5D (e.g., human GPRC5D or cynomolgus GPRC5D) compared with JNJ-64407564 and/or RG6234.

In certain embodiments, the bispecific antibody or antigen-binding fragment which competes for binding to CD3 and GPRC5D with the antibody or antigen-binding fragment thereof provided herein is not JNJ-64407564 or RG6234.

“JNJ-64407564” as used here refers to a bispecific antibody or antigen-binding fragment thereof comprising a heavy chain variable region targeting GPRC5D having an amino acid sequence of SEQ ID NO: 5, a light chain variable region targeting GPRC5D having an amino acid sequence of SEQ ID NO: 6, a heavy chain variable region targeting CD3 having an amino acid sequence of SEQ ID NO: 7, and a light chain variable region targeting GPRC5D having an amino acid sequence of SEQ ID NO: 8. The CDR sequences were bold and underlined in SEQ ID NO: 5, SEQ ID NO: 6, SEQ ID NO: 7 and SEQ ID NO: 8, respectively.

(SEQ ID NO: 5)
QVQLVQSGAEVKKPGASVKVSCKASGYSFTGYTMNWVRQAPGQGLEWMG
LINPYNSDTNYAQKLQGRVTMTTDTSTSTAYMELRSLRSDDTAVYYCAR
VALRVALDYWGQGTLVTVSS
(SEQ ID NO: 6)
DIQMTQSPSSLSASVGDRVTITCKASQNVATHVGWYQQKPGKAPKRLIY
SASYRYSGVPSRFSGSGSGTEFTLTISNLQPEDFATYYCQQYNRYPYTF
GQGTKLEIK
(SEQ ID NO: 7)
EVQLVESGGGLVQPGGSLRLSCAASGFTFNTYAMNWVRQAPGKGLEWVA
RIRSKYNNYATYYAASVKGRFTISRDDSKNSLYLQMNSLKTEDTAVYYC
ARHGNFGNSYVSWFAYWGQGTLVTVSS
(SEQ ID NO: 8)
QTVVTQEPSLTVSPGGTVTLTCRSSTGAVTTSNYANWVQQKPGQAPRGL
IGGTNKRAPGTPARFSGSLLGGKAALTLSGVQPEDEAEYYCALWYSNLW
VFGGGTKLTVL

“RG6234” as used here refers to a bispecific antibody or antigen-binding fragment thereof comprising a heavy chain variable region targeting GPRC5D having an amino acid sequence of SEQ ID NO: 9, a light chain variable region targeting GPRC5D having an amino acid sequence of SEQ ID NO: 10, a heavy chain variable region targeting CD3 having an amino acid sequence of SEQ ID NO: 11, and a light chain variable region targeting GPRC5D having an amino acid sequence of SEQ ID NO: 12. The CDR sequences were bold and underlined in SEQ ID NO: 9, SEQ ID NO: 10, SEQ ID NO: 11 and SEQ ID NO: 12, respectively.

(SEQ ID NO: 9)
EVQLVESGGGLVQPGRSLKLSCAASGFSFSNYGMAWVRQAATKGLEWVA
SISTGGGNTYYRDSVKGRFIVSRDNAKNTQYLQMDSLRSEDTATYYCTR
HDRGGLYWGQGVMVTVSS
(SEQ ID NO: 10)
DIVMTQAPLSVSVTPGESASISCRSSKSLLHSNGITYVYWYFQKPGKSP
QVLIYRMSNLASGVPDRFSGSGSETDFTLKISRVEAEDVGIYHCGQLLE
NPYTFGAGTELELK
(SEQ ID NO: 11)
EVQLLESGGGLVQPGGSLRLSCAASGFTFSTYAMNWVRQAPGKGLEWVS
RIRSKYNNYATYYADSVKGRFTISRDDSKNTLYLQMNSLRAEDTAVYYC
VRHGNFGNSYVSWFAYWGQGTLVTVSS
(SEQ ID NO: 12)
QAVVTQEPSLTVSPGGTVTLTCGSSTGAVTTSNYANWVQEKPGQAFRGL
IGGTNKRAPGTPARFSGSLLGGKAALTLSGAQPEDEAEYYCALWYSNLW
VFGGGTKLTVL

### Antibody Variants

The antibodies and antigen-binding fragments thereof provided herein also encompass various variants of the antibody sequences provided herein.

In certain embodiments, the antibody variants comprise one or more amino acid residue substitutions or modifications yet retains specific binding affinity to the first binding moiety. In certain embodiments, at least one of the substitutions or modifications is within one or more of the CDR sequences of the VH region or VL region. In certain embodiments, at least one of the substitutions or modifications is within one or more of the non-CDR sequences of the VH region or VL region. In certain embodiments, the antibodies or antigen-binding fragments thereof provided herein further comprise one or more non-natural amino acid (NNAA) substitution. In certain embodiments, the NNAA is capable of being conjugated.

In certain embodiments, at least one of the substitutions or modifications is within one or more of the CDR sequences of the VH region or VL region of the first binding moiety. In certain embodiments, at least one of the substitutions or modifications is within one or more of the non-CDR sequences of the VH region or VL region of the first binding moiety. For example, the first binding moieties of the antibody variants comprise one or more amino acid residue substitutions or modifications within one or more of the CDR sequences provided in Table 22A, Table 22B, Table 22C, Table 22D and Table 22E above, one or more of the non-CDR sequences of the heavy chain variable region or light chain variable region provided in Table 23 above, and/or the constant region (e.g., Fc region). Such variants retain binding specificity to CD3 of their parent antibodies, but have one or more desirable properties conferred by the modification(s) or substitution(s). For example, the antibody variants may have improved antigen-binding affinity, improved glycosylation pattern, reduced risk of glycosylation, reduced deamination, enhanced effector function(s), improved FcRn receptor binding, increased pharmacokinetic half-life, pH sensitivity, and/or compatibility to conjugation (e.g., one or more introduced cysteine residues), etc.

In certain embodiments, the antibody variants comprise one or more amino acid residue substitutions or modifications yet retains specific binding affinity to the second binding moiety. In certain embodiments, at least one of the substitutions or modifications is within one or more of the CDR sequences of the VH region or VL region. In certain embodiments, at least one of the substitutions or modifications is within one or more of the non-CDR sequences of the VH region or VL region. In certain embodiments, the antibodies or antigen-binding fragments thereof provided herein further comprise one or more non-natural amino acid (NNAA) substitution. In certain embodiments, the NNAA is capable of being conjugated.

In certain embodiments, at least one of the substitutions or modifications is within one or more of the CDR sequences of the VH region or VL region of the second binding moiety. In certain embodiments, at least one of the substitutions or modifications is within one or more of the non-CDR sequences of the VH region or VL region of the second binding moiety. For example, the second binding moieties of the antibody variants comprise one or more amino acid residue substitutions or modifications within one or more of the CDR sequences targeting GPRC5D provided in Table 25 above, one or more of the one or more of the CDR sequences targeting GD2 provided in Table 28 above, one or more of the one or more of the CDR sequences targeting LILRB4 provided in Table 46 above, one or more of the one or more of the CDR sequences targeting CD19 provided in Table 51 above, one or more of the one or more of the CDR sequences targeting CD20 provided in Table 54 above, non-CDR sequences of the heavy chain variable region or light chain variable region targeting GPRC5D provided in Table 26 above, non-CDR sequences of the heavy chain variable region or light chain variable region targeting GD2 provided in Table 29 above, non-CDR sequences of the heavy chain variable region or light chain variable region targeting LILRB4 provided in Table 47 above, non-CDR sequences of the heavy chain variable region or light chain variable region targeting CD19 provided in Table 52 above, non-CDR sequences of the heavy chain variable region or light chain variable region targeting CD20 provided in Table 55 above, and/or the constant region (e.g., Fc region). Such variants retain binding specificity to the binding antigen other than CD3 of their parent antibodies, but have one or more desirable properties conferred by the modification(s) or substitution(s). For example, the antibody variants may have improved antigen-binding affinity, improved glycosylation pattern, reduced risk of glycosylation, reduced deamination, enhanced effector function(s), improved FcRn receptor binding, increased pharmacokinetic half-life, pH sensitivity, and/or compatibility to conjugation (e.g., one or more introduced cysteine residues), etc.

The parent antibody sequence may be screened to identify suitable or preferred residues to be modified or substituted, using methods known in the art, for example, “alanine scanning mutagenesis” (see, for example, Cunningham and Wells (1989) Science, 244:1081-1085). Briefly, target residues (e.g., charged residues such as Arg, Asp, His, Lys, and Glu) can be identified and replaced by a neutral or negatively charged amino acid (e.g., alanine or polyalanine), and the modified antibodies are produced and screened for the interested property. If substitution at a particular amino acid location demonstrates an interested functional change, then the position can be identified as a potential residue for modification or substitution. The potential residues may be further assessed by substituting with a different type of residue (e.g., cysteine residue, positively charged residue, etc.).

### Affinity Variants

Affinity variants of antibodies may contain modifications or substitutions within one or more CDR sequences targeting the first binding moiety provided in Table 22A, Table 22B, Table 22C, Table 22D and Table 22E above, one or more FR sequences, or the heavy or light chain variable region sequences provided in Table 23 above. FR sequences can be readily identified by a person skilled in the art based on the CDR sequences in Table 22A, Table 22B, Table 22C, Table 22D and Table 22E above and variable region sequences in Table 23 above, as it is well-known in the art that a CDR region is flanked by two FR regions in the variable region. The affinity variants retain specific binding affinity to the first binding moiety of the parent antibody, or even have improved CD3 specific binding affinity to the first binding moiety over the parent antibody. In certain embodiments, at least one (or all) of the substitution(s) in the CDR sequences, FR sequences, or variable region sequences comprises a conservative substitution.

A person skilled in the art will understand that in the CDR sequences targeting the first binding moiety provided herein, and variable region sequences provided herein, one or more amino acid residues may be substituted yet the resulting antibody or antigen-binding fragment still retain the binding affinity or binding capacity to the first binding moiety, or even have an improved binding affinity or capacity. Various methods known in the art can be used to achieve this purpose. For example, a library of antibody variants (such as Fab or scFv variants) can be generated and expressed with phage display technology, and then screened for the binding affinity to human CD3. For another example, computer software can be used to virtually simulate the binding of the antibodies to human CD3, and identify the amino acid residues on the antibodies which form the binding interface. Such residues may be either avoided in the substitution so as to prevent reduction in binding affinity, or targeted for substitution to provide for a stronger binding.

In certain embodiments, the humanized antibody or antigen-binding fragment thereof provided herein comprises one or more amino acid residue substitutions within one or more of the CDR sequences, and/or one or more of the FR sequences. In certain embodiments, an affinity variant comprises no more than 20, 15, 10, 9, 8, 7, 6, 5, 4, 3, 2, or 1 substitutions in the CDR sequences and/or FR sequences in total.

In certain embodiments, the first binding moiety of the antibodies or antigen-binding fragments thereof provided herein comprise 1, 2, or 3 CDR sequences having at least 80% (e.g., at least 85%, 86%, 87%, 88%, 89%, 90%, 91%, 92%, 93%, 94%, 95%, 96%, 97%, 98%, or 99%) sequence identity to that (or those) listed in Table 22A, Table 22B, Table 22C, Table 22D and Table 22E above yet retaining the specific binding affinity to CD3 at a level similar to or even higher than its parent antibody.

In certain embodiments, the first binding moiety of the antibodies or antigen-binding fragments thereof provided herein comprise one or more variable region sequences having at least 80% (e.g., at least 85%, 86%, 87%, 88%, 89%, 90%, 91%, 92%, 93%, 94%, 95%, 96%, 97%, 98%, or 99%) sequence identity to that (or those) listed in Table 23 above yet retaining the specific binding affinity to CD3 at a level similar to or even higher than its parent antibody. In some embodiments, a total of 1 to 10 amino acids have been substituted, inserted, or deleted in a variable region sequence listed in Table 23 above. In some embodiments, the substitutions, insertions, or deletions occur in regions outside the CDRs (e.g., in the FRs).

Affinity variants of antibodies may contain modifications or substitutions within one or more CDR sequences targeting the second binding moiety provided in Table 25, Table 28, Table 46, Table 51 or Table 54 above, one or more FR sequences, or the heavy or light chain variable region sequences provided in Table 26, Table 29, Table 47, Table 52 or Table 55 above. FR sequences can be readily identified by a person skilled in the art based on the CDR sequences in Table 25, Table 28, Table 46, Table 51 or Table 54 above and variable region sequences in Table 26, Table 29, Table 47, Table 52 or Table 55 above, as it is well-known in the art that a CDR region is flanked by two FR regions in the variable region. The affinity variants retain specific binding affinity to the second binding moiety of the parent antibody, or even have improved specific binding affinity to the second binding moiety over the parent antibody. In certain embodiments, at least one (or all) of the substitution(s) in the CDR sequences, FR sequences, or variable region sequences comprises a conservative substitution.

A person skilled in the art will understand that in the CDR sequences targeting the second binding moiety provided in Table 25, Table 28, Table 46, Table 51 or Table 54 above, and variable region sequences provided in Table 26, Table 29, Table 47, Table 52 or Table 55 above, one or more amino acid residues may be substituted yet the resulting antibody or antigen-binding fragment still retain the binding affinity or binding capacity to the first binding moiety, or even have an improved binding affinity or capacity. Various methods known in the art can be used to achieve this purpose. For example, a library of antibody variants (such as Fab or scFv variants) can be generated and expressed with phage display technology, and then screened for the binding affinity to human GPRC5D, GD2, LILRB4, CD19 or CD20. For another example, computer software can be used to virtually simulate the binding of the antibodies to human GPRC5D, GD2, LILRB4, CD19 or CD20, and identify the amino acid residues on the antibodies which form the binding interface. Such residues may be either avoided in the substitution so as to prevent reduction in binding affinity, or targeted for substitution to provide for a stronger binding.

In certain embodiments, the humanized antibody or antigen-binding fragment thereof provided herein comprises one or more amino acid residue substitutions within one or more of the CDR sequences, and/or one or more of the FR sequences. In certain embodiments, an affinity variant comprises no more than 20, 15, 10, 9, 8, 7, 6, 5, 4, 3, 2, or 1 substitutions in the CDR sequences and/or FR sequences in total.

In certain embodiments, the second binding moiety of the antibodies or antigen-binding fragments thereof provided herein comprise 1, 2, or 3 CDR sequences having at least 80% (e.g., at least 85%, 86%, 87%, 88%, 89%, 90%, 91%, 92%, 93%, 94%, 95%, 96%, 97%, 98%, or 99%) sequence identity to that (or those) listed in Table 25 and/or Table 28 and/or Table 46 above yet retaining the specific binding affinity to GPRC5D, GD2, LILRB4, CD19 or CD20 at a level similar to or even higher than its parent antibody.

In certain embodiments, the second binding moiety of the antibodies or antigen-binding fragments thereof comprise one or more variable region sequences having at least 80% (e.g., at least 85%, 86%, 87%, 88%, 89%, 90%, 91%, 92%, 93%, 94%, 95%, 96%, 97%, 98%, or 99%) sequence identity to that (or those) listed in Table 26 and/or Table 29 and/or Table 47 above yet retaining the specific binding affinity to GPRC5D, GD2, LILRB4, CD19 or CD20 at a level similar to or even higher than its parent antibody. In some embodiments, a total of 1 to 10 amino acids have been substituted, inserted, or deleted in a variable region sequence listed in Table 26 and/or Table 29 and/or Table 47 above. In some embodiments, the substitutions, insertions, or deletions occur in regions outside the CDRs (e.g., in the FRs).

### Glycosylation Variants

The bispecific or multi-specific antibodies or antigen-binding fragments thereof provided herein also encompass glycosylation variants, which can be obtained to either increase or decrease the extent of glycosylation of the antibodies or antigen-binding fragments thereof.

The antibodies or antigen-binding fragments thereof provided herein may comprise one or more modifications that introduce or remove a glycosylation site. A glycosylation site is an amino acid residue with a side chain to which a carbohydrate moiety (e.g., an oligosaccharide structure) can be attached. Glycosylation of antibodies is typically either N-linked or O-linked. N-linked refers to the attachment of the carbohydrate moiety to the side chain of an asparagine residue, for example, an asparagine residue in a tripeptide sequence such as asparagine-X-serine and asparagine-X-threonine, where X is any amino acid except proline. O-linked glycosylation refers to the attachment of one of the sugars N-aceylgalactosamine, galactose, or xylose to a hydroxyamino acid, most commonly to serine or threonine. Removal of a native glycosylation site can be conveniently accomplished, for example, by altering the amino acid sequence such that one of the above-described tripeptide sequences (for N-linked glycosylation sites) or serine or threonine residues (for O-linked glycosylation sites) present in the sequence is substituted. A new glycosylation site can be created in a similar way by introducing such a tripeptide sequence or serine or threonine residue.

### Cysteine-Engineered Variants

The bispecific or multi-specific antibodies or antigen-binding fragments thereof provided herein also encompass cysteine-engineered variants, which comprise one or more introduced free cysteine amino acid residues.

A free cysteine residue is one which is not part of a disulfide bridge. A cysteine-engineered variant is useful for conjugation with for example, a cytotoxic and/or imaging compound, a label, or a radioisoptype among others, at the site of the engineered cysteine, through for example a maleimide or haloacetyl. Methods for engineering antibodies or antigen-binding fragments thereof to introduce free cysteine residues are known in the art, see, for example, WO2006/034488.

### Fc Variants

The bispecific or multi-specific antibodies and antigen-binding fragments provided herein also encompass an Fc variant, which comprises one or more amino acid residue modifications or substitutions at its Fc region and/or hinge region.

In certain embodiments, the bispecific or multi-specific antibodies or antigen-binding fragments thereof provided herein comprise one or more amino acid substitution(s) that improves pH-dependent binding to neonatal Fc receptor (FcRn). Such a variant can have an extended pharmacokinetic half-life, as it binds to FcRn at acidic pH which allows it to escape from degradation in the lysosome and then be translocated and released out of the cell. Methods of engineering an antibody or antigen-binding fragment thereof to improve binding affinity with FcRn are well-known in the art, see, for example, Vaughn, D. et al., Structure, 6 (1): 63-73, 1998; Kontermann, R. et al., Antibody Engineering, Volume 1, Chapter 27: Engineering of the Fc region for improved PK, published by Springer, 2010; Yeung, Y. et al., Cancer Research, 70:3269-3277 (2010); and Hinton, P. et al., J. Immunology, 176:346-356 (2006).

In certain embodiments, the bispecific or multi-specific antibodies or antigen-binding fragments thereof provided herein comprise one or more amino acid substitution(s) that alters ADCC. Certain amino acid residues at CH2 domain of the Fc region can be substituted to provide for enhanced ADCC activity. Alternatively or additionally, carbohydrate structures on the antibody can be changed to enhance ADCC activity. Methods of altering ADCC activity by antibody engineering have been described in the art, see for example, Shields R L. Et al., J Biol Chem. 2001. 276 (9): 6591-604; Idusogie E E. Et al., J Immunol. 2000.164 (8): 4178-84; Steurer W. et al., J Immunol. 1995, 155 (3): 1165-74; Idusogie E E. Et al., J Immunol. 2001, 166 (4): 2571-5; Lazar G A. Et al., PNAS, 2006, 103 (11): 4005-4010; Ryan M C. Et al., Mol. Cancer Ther., 2007, 6:3009-3018; Richards J O,. Et al., Mol Cancer Ther. 2008, 7 (8): 2517-27; Shields R. L. et al., J. Biol. Chem, 2002, 277:26733-26740; Shinkawa T. et al., J. Biol. Chem, 2003, 278:3466-3473.

In certain embodiments, the bispecific or multi-specific antibodies or antigen-binding fragments thereof comprise one or more amino acid substitution(s) that alters CDC, for example, by improving or diminishing C1q binding and/or CDC (see, for example, WO99/51642; Duncan & Winter Nature 322:738-40 (1988); U.S. Pat. Nos. 5,648,260; 5,624,821); and WO94/29351 concerning other examples of Fc region variants. One or more amino acids selected from amino acid residues 329, 331 and 322 of the Fc region can be replaced with a different amino acid residue to alter C1q binding and/or enhance CDC (see, U.S. Pat. No. 6,194,551 by Idusogie et al.). One or more amino acid substitution(s) can also be introduced to alter the ability of the antibody to fix complement (see PCT Publication WO 94/29351 by Bodmer et al.).

In certain embodiments, the bispecific or multi-specific antibodies or antigen-binding fragments thereof provided herein comprise one or more amino acid substitution(s) in human immunoglobulin (e.g., IgG1) at position 234 and/or 235 (according to EU numbering). In certain embodiments, the bispecific or multi-specific antibodies or antigen-binding fragments thereof provided herein comprise two amino acid substitutions in human immunoglobulin (e.g., IgG1) at positions 234 and 235 (according to EU numbering). In certain embodiments, the bispecific or multi-specific antibodies or antigen-binding fragments thereof provided herein comprise L234A and L235A (according to EU numbering) amino acid substitutions.

In certain embodiments, the bispecific or multi-specific antibodies or antigen-binding fragments thereof provided herein comprise one or more amino acid substitution(s) in human immunoglobulin (e.g., IgG4) at position 228 (according to EU numbering). In certain embodiments, the first binding moiety of the antibodies or antigen-binding fragments thereof provided herein comprise S228P (according to EU numbering) amino acid substitution.

In certain embodiments, the bispecific or multi-specific antibodies or antigen-binding fragments thereof provided herein comprise one or more amino acid substitution(s) in the interface of the Fc region to facilitate and/or promote heterodimerization. These modifications comprise introduction of a protuberance into a first Fc polypeptide and a cavity into a second Fc polypeptide, wherein the protuberance can be positioned in the cavity so as to promote interaction of the first and second Fc polypeptides to form a heterodimer or a complex. Methods of generating antibodies with these modifications are known in the art, e.g., as described in U.S. Pat. No. 5,731,168.

In certain embodiments, the bispecific or multi-specific antibodies or antigen-binding fragments thereof provided herein comprise an amino acid substitution at position 366 (according to EU numbering) of a first Fc polypeptide, and comprises one, two or three amino acid substitutions at one, two or three positions of 366, 368, and 407 (according to EU numbering) of a second Fc polypeptide. In certain embodiments, the bispecific or multi-specific antibodies or antigen-binding fragments thereof provided herein comprise a T366W substitution (according to EU numbering) of a first Fc polypeptide, and comprise T366S+L368A+Y407V substitutions (according to EU numbering) of a second Fc polypeptide.

In certain embodiments, the bispecific or multi-specific antibodies or antigen-binding fragments thereof provided herein further comprise one or more amino acid substitution(s) of a first Fc polypeptide, and comprise one or more amino acid substitution(s) of a second Fc polypeptide, so as to introduce a non-natural disulfide bond between the two Fc polypeptides. For example, the bispecific or multi-specific antibodies or antigen-binding fragments thereof provided herein comprise an amino acid substitution at position 354 (according to EU numbering) of a first Fc polypeptide, and comprise an amino acid substitution at position 349 (according to EU numbering) of a second Fc polypeptide. In certain embodiments, the bispecific or multi-specific antibodies or antigen-binding fragments thereof provided herein comprise S354C substitution (according to EU numbering) of a first Fc polypeptide, and comprise Y349C substitution (according to EU numbering) of a second Fc polypeptide.

### Antigen-binding Fragments

Provided herein are also bispecific or multi-specific antigen-binding fragments. Various types of antigen-binding fragments are known in the art and can be developed based on the bispecific or multi-specific antibodies provided herein, including for example, the exemplary antibodies whose CDRs are shown in Table 22A, Table 22B, Table 22C, Table 22D and Table 22E above, and variable sequences are shown in Table 23 above, and their different variants (such as affinity variants, glycosylation variants, Fc variants, cysteine-engineered variants and so on).

In certain embodiments, an bispecific or multi-specific antigen-binding fragment provided herein is a diabody, a Fab, a Fab′, a F(ab′)₂, a Fd, an Fv fragment, a disulfide stabilized Fv fragment (dsFv), a (dsFv) 2, a bispecific dsFv (dsFv-dsFv′), a disulfide stabilized diabody (ds diabody), a single-chain antibody molecule (scFv), an scFv dimer (bivalent diabody), a multi-specific antibody, a camelized single domain antibody, a nanobody, a domain antibody, or a bivalent domain antibody.

Various techniques can be used for the production of such antigen-binding fragments. Illustrative methods include, enzymatic digestion of intact antibodies (see, e.g., Morimoto et al., Journal of Biochemical and Biophysical Methods 24:107-117 (1992); and Brennan et al., Science, 229:81 (1985)), recombinant expression by host cells such as E. Coli (e.g., for Fab, Fv and ScFv antibody fragments), screening from a phage display library as discussed above (e.g., for ScFv), and chemical coupling of two Fab′-SH fragments to form F(ab′)₂ fragments (Carter et al., Bio/Technology 10:163-167 (1992)). Other techniques for the production of antibody fragments will be apparent to a person skilled in the art.

In certain embodiments, the antigen-binding fragment is a scFv. Generation of scFv is described in, for example, WO 93/16185; U.S. Pat. Nos. 5,571,894; and 5,587,458. ScFv may be fused to an effector protein at either the amino or the carboxyl terminus to provide for a fusion protein (see, for example, Antibody Engineering, ed. Borrebaeck).

In certain embodiments, the first binding moiety is an anti-CD3 scFv domain, and the second binding moiety is a Fab domain; or the first binding moiety is an anti-CD3 Fab domain, and the second binding moiety is a scFv domain.

In certain embodiments, the first binding moiety is linked to the second binding moiety via a linker. In certain embodiments, the first binding moiety is directly linked (i.e., without a linker) to the second binding moiety. In certain embodiments, the N-terminus of the first binding moiety is linked to the C-terminus of the second binding moiety directly or via the linker.

In certain embodiments, the bispecific or multi-specific antibodies or antigen-binding fragments thereof comprise, from N-terminus to C-terminus, a first chain comprising a VH region from the second binding moiety, a CH1 region, a VH region from the first binding moiety and a VL region from the first binding moiety. In certain embodiments, the bispecific or multi-specific antibodies or antigen-binding fragments thereof comprise, from N-terminus to C-terminus, a first chain comprising a VH region from the second binding moiety, a CH1 region, a VH region from the first binding moiety, a VL region from the first binding moiety, a CH2 region and a CH3 region. In certain embodiments, the bispecific or multi-specific antibodies or antigen-binding fragments thereof comprise, from N-terminus to C-terminus, a first chain comprising a VH region from the second binding moiety, a CH1 region, a VL region from the first binding moiety and a VH region from the first binding moiety. In certain embodiments, the bispecific or multi-specific antibodies or antigen-binding fragments thereof comprise, from N-terminus to C-terminus, a first chain comprising a VH region from the second binding moiety, a CH1 region, a VL region from the first binding moiety, a VH region from the first binding moiety, a CH2 region and a CH3 region.

In certain embodiments, the VH region from the first binding moiety and the VL region from the first binding moiety are directly linked or linked via a linker. In certain embodiments, the linker is a linker comprising glycine and serine, e.g., a (GGGGS)₂ linker. For example, the CD3 binding moiety is an antigen-binding fragment thereof (e.g., a scFv), wherein the CD3 binding moiety is linked to the GPRC5D binding moiety directly or via the (GGGGS)₂ linker.

In certain embodiments, the bispecific or multi-specific antibodies or antigen-binding fragments thereof provided herein further comprise a second chain comprising a VL region from the second binding moiety and a CL region. In certain embodiments, the bispecific or multi-specific antibodies or antigen-binding fragments thereof further comprise a second chain comprising, from N-terminus to C-terminus, a VL region from the second binding moiety and a CL region. In certain embodiments, the VH region from the second binding moiety of the first chain and the VL region of the second chain are associated to form a binding domain.

In certain embodiments, the bispecific or multi-specific antibodies or antigen-binding fragments thereof provided herein further comprise a third chain comprising a VH region from the third binding moiety and a CH1 region. In certain embodiments, the third chain comprises, from N-terminus to C-terminus, a VH region from the third binding moiety and a CH1 region. In certain embodiments, the third chain comprises, from N-terminus to C-terminus, a VH region from the third binding moiety, a CH1 region, a CH2 region and a CH3 region.

In certain embodiments, the bispecific or multi-specific antibodies or antigen-binding fragments thereof provided herein further comprise a fourth chain comprising a VL region from the third binding moiety and a CL region. In certain embodiments, the fourth chain comprises, from N-terminus to C-terminus, a VL region from the third binding moiety and a CL region. In certain embodiments, the VH region from the third binding moiety of the third chain and the VL region of the fourth chain are associated to form a binding domain.

In certain embodiments, the bispecific or multi-specific antibodies or antigen-binding fragments thereof provided herein comprise the first chain as described above, the second chain as described above, the third chain as described above and the fourth chain as described above, wherein the first chain and the third chain are covalently linked, e.g., through a disulfide bond.

In certain embodiments, the bispecific or multi-specific antibodies or antigen-binding fragments thereof provided herein are in a construct of full-length antibody targets a first epitope, except that a scFv domain targeting a second epitope is inserted to link one of the two Fab domains to a first Fc region, and the other Fab domain is still linked to a second Fc region, wherein the first and second Fc regions are covalently linked.

In certain embodiments, the bispecific or multi-specific antibodies or antigen-binding fragments thereof provided herein are bivalent, tetravalent, hexavalent, or multivalent. Any molecule being more than bivalent is considered multivalent, encompassing for example, trivalent, tetravalent, hexavalent, and so on.

A bivalent molecule can be monospecific if the two binding sites are both specific for binding to the same antigen or the same epitope. This, in certain embodiments, provides for stronger binding to the antigen or the epitope than a monovalent counterpart. Similar, a multivalent molecule may also be monospecific. In certain embodiments, in a bivalent or multivalent antigen-binding moiety, the first valent of binding site and the second valent of binding site are structurally identical (i.e., having the same sequences), or structurally different (i.e., having different sequences albeit with the same specificity).

A bivalent can also be bispecific, if the two binding sites are specific for different antigens or epitopes. This also applies to a multivalent molecule. For example, a trivalent molecule can be bispecific when two binding sites are monospecific for a first antigen (or epitope) and the third binding site is specific for a second antigen (or epitope).

### Conjugates

In some embodiments, the antibodies or antigen-binding fragments thereof provided herein further comprise one or more conjugate moieties. The conjugate moiety can be linked to the antibodies or antigen-binding fragments thereof. A conjugate moiety is a moiety that can be attached to the antibody or antigen-binding fragment thereof. It is contemplated that a variety of conjugate moieties may be linked to the antibodies or antigen-binding fragments thereof provided herein (see, for example, “Conjugate Vaccines”, Contributions to Microbiology and Immunology, J. M. Cruse and R. E. Lewis, Jr. (eds.), Carger Press, New York, (1989)). These conjugate moieties may be linked to the antibodies or antigen-binding fragments thereof by covalent binding (e.g., disulfide bond), affinity binding, intercalation, coordinate binding, complexation, association, blending, or addition, among other methods. In some embodiments, the antibodies or antigen-binding fragments thereof can be linked to one or more conjugates via a linker or a crosslinking agent. The linker or crosslinking agent comprises a reactive chemical group that can react with the anti-CD3 antibodies or antigen-binding fragments thereof. The reactive chemical groups can be N-succinimidyl esters and N-sulfosuccinimidyl esters. Additionally, the linker comprises a reactive chemical group, which can be a dithiopyridyl group that can react with the drug to form a disulfide bond. Linker molecules include, for example, N-succinimidyl 4-(maleimidomethyl)cyclohexanecarboxylate (SMCC), N-succinimidyl 3-(2-pyridyldithio) propionate (SPDP) (see, e.g., Carlsson et al., Biochem. J., 173:723-737 (1978)), N-succinimidyl 4-(2-pyridyldithio) butanoate (SPDB) (see, e.g., U.S. Pat. No. 4,563,304), N-succinimidyl 4-(2-pyridyldithio) 2-sulfobutanoate (sulfo-SPDB) (see US Publication No. 20090274713), N-succinimidyl 4-(2-pyridyldithio) pentanoate (SPP) (see, e.g., CAS Registry number 341498-08-6), 2-iminothiolane, or acetylsuccinic anhydride. For example, the antibody or cell binding agent can be modified with crosslinking reagents and the antibody or cell binding agent containing free or protected thiol groups thus derived is then reacted with a disulfide- or thiol-containing maytansinoid to produce conjugates. The conjugates can be purified by chromatography, including but not limited to HPLC, size-exclusion, adsorption, ion exchange and affinity capture, dialysis or tangential flow filtration.

In certain embodiments, the antibodies or antigen-binding fragments thereof provided herein may be engineered to contain specific sites outside the epitope binding portion that may be utilized for binding to one or more conjugate moieties. For example, such a site may include one or more reactive amino acid residues, such as for example cysteine or histidine residues, to facilitate covalent linkage to a conjugate moiety. In certain embodiments, the NNAA substitution is capable of being conjugated.

In certain embodiments, the antibodies or antigen-binding fragments thereof provided herein may be linked to a conjugate moiety indirectly, or through another conjugate moiety. In certain embodiments, the conjugate moiety is covalently attached either directly or via a linker. For example, the antibodies or antigen-binding fragments thereof provided herein may be conjugated to biotin, then indirectly conjugated to a second conjugate that is conjugated to avidin. In some embodiments, the conjugate moiety comprises a clearance-modifying agent (e.g., a polymer such as PEG which extends half-life), a chemotherapeutic agent, a toxin, a radioactive isotope, a lanthanide, a detectable label (e.g., a luminescent label, a fluorescent label, an enzyme-substrate label), a DNA-alkylator, a topoisomerase inhibitor, a tubulin-binder, a purification moiety or other anticancer drugs (e.g., agonist of toll-like receptor 7 (TLR-7), TLR-8 and/or TLR-9, siRNA, antibody or antigen-binding fragments thereof, a peptide (such as a short peptide), etc.).

A “toxin” can be any agent that is detrimental to cells or that can damage or kill cells. Examples of toxin include, without limitation, taxol, 191ercuri, CC-1065 and CC-1065 analogs, duocarmycins and duocarmycin analogs, enediynes such as calicheamicins, dolastatin and dolastatin analogs including auristatins, tomaymycin derivatives, leptomycin derivatives, cisplatin, carboplatin, daunorubicin, doxorubicin, vincristine, vinblastine, melphalan, mitomycin C, chlorambucil and morpholino doxorubicin, cytochalasin B, gramicidin D, ethidium bromide, emetine, mitomycin, etoposide, tenoposide, vincristine, MMAE, MMAF, DM1, DM4, vinblastine, 191 ercurial191, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin and analogs thereof, antimetabolites (e.g., methotrexate, 6-mercaptopurine, 6-thioguanine, cytarabine, 5-fluorouracil 191ercurial191e), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), anti-mitotic agents (e.g., vincristine and vinblastine), a topoisomerase inhibitor, and a tubulin-binders.

Examples of detectable label may include a fluorescent label (e.g., fluorescein, rhodamine, dansyl, phycoerythrin, or Texas Red), an enzyme-substrate label (e.g., horseradish peroxidase, alkaline phosphatase, luceriferases, glucoamylase, lysozyme, saccharide oxidases or β-D-galactosidase), a radioisotope (e.g., ¹²³I, ¹²⁴I, ¹²⁵I, ¹³¹I, ³⁵S, ³H, ¹¹¹In, ¹¹²In, ¹⁴C, ⁶⁴Cu, ⁶⁷Cu, ⁸⁶Y, ⁸⁸Y, ⁹⁰Y, ¹⁷⁷Lu, ²¹¹At, ¹⁸⁶Re, ¹⁸⁸Re, ¹⁵³Sm, ²¹²Bi, and ³²P, other lanthanides), a luminescent label, a chromophoric moiety, digoxigenin, biotin/avidin, a DNA molecule or gold for detection.

In certain embodiments, the conjugate moiety can be a clearance-modifying agent which helps increase half-life of the antibody. Illustrative examples include water-soluble polymers, such as PEG, carboxymethylcellulose, dextran, polyvinyl alcohol, polyvinyl pyrrolidone, copolymers of ethylene glycol/propylene glycol, and the like. The polymer may be of any molecular weight, and may be branched or unbranched. The number of polymers attached to the antibody may vary, and if more than one polymer are attached, they can be the same or different molecules.

In certain embodiments, the conjugate moiety can be a purification moiety such as a magnetic bead.

In certain embodiments, the antibody or an antigen-binding fragment thereof provided herein is used as a base for a conjugate.

In certain embodiments, the antibody or an antigen-binding fragment thereof provided herein is conjugated to a signal peptide. A signal peptide (sometimes referred to as signal sequence, leader sequence or leader peptide) can be used to facilitate secretion and isolation of the antibodies or antigen-binding fragments thereof provided herein. Signal peptides are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the invention pertains to the described polypeptides having a signal sequence, as well as to polypeptides from which the signal sequence has been proteolytically cleaved (i.e., the cleavage products). In one embodiment, a nucleic acid sequence encoding a signal sequence can be operably linked in an expression vector to a protein of interest, such as a protein which is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by art recognized methods. Alternatively, the signal sequence can be linked to the protein of interest using a sequence which facilitates purification, such as with a GST domain.

### Chimeric Antigen Receptor

In certain embodiments, the present disclosure provides a chimeric antigen receptor comprising the antibody or an antigen-binding fragment thereof provided herein, a transmembrane region and an intracellular signal region.

The term “chimeric antigen receptor” or “CAR” or “CARs” as used herein refers to engineered receptors, which graft an antigen specificity onto cells (for example, T cells such as naïve T cells, central memory T cells, effector memory T cells, regulatory T cells, natural killer cells, or combination thereof). CARs are also known as artificial T-cell receptors, chimeric T-cell receptors or chimeric immunoreceptors. In some embodiments, CARs comprise an antigen-specific targeting region (for example, the antigen-binding fragments of the anti-CD3 antibody as provided herein), an extracellular region, a transmembrane region, one or more co-stimulatory regions, and an intracellular signal region.

In some embodiments, the antigen-specific targeting region is an scFv. In some embodiments, the transmembrane region comprises a transmembrane region of CD3, CD4, CD8 or CD28. In some embodiments, the co-stimulatory region comprises a co-stimulatory domain of CD28, ICOS, CD27, 4-1BB, OX40 and CD40L. In some embodiments, the intracellular signal region is selected from the group consisting of: an intracellular signal region sequence of CD3, Fc γ RI, CD27, CD28, CD137, CD134, MyD88, CD40, CD278, TLRs, or a combination thereof.

In some embodiments, the CARs are grafted onto various cells, for example, allogeneic cells, autologous cells or xenogeneic cells.

The term “allogeneic cell” as used herein refers to any cells derived from a different subject of the same species.

The term “autologous cell” as used herein refers to any cells derived from the same subject into which they are later to be re-introduced.

The term “xenogeneic cell” as used herein refers to any cells derived from a different subject of a different species.

In some embodiments, the CARs are grafted on immune effector cells, for example, T cells, natural killer cells, macrophage cells, tumor-infiltrating lymphocytes, etc.

### Polynucleotides and Recombinant Methods

The present disclosure provides isolated polynucleotides that encode the bispecific or multi-specific antibodies or antigen-binding fragments thereof and/or the chimeric antigen receptors provided herein. The term “nucleic acid” or “polynucleotide” as used herein refers to deoxyribonucleic acids (DNA) or ribonucleic acids (RNA) and polymers thereof in either single- or double-stranded form. Unless otherwise indicated, a particular polynucleotide sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (see Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

DNA encoding the antibody or antigen-binding fragment thereof, or the chimeric antigen receptor provided herein is readily isolated and sequenced using conventional procedures (e.g., by using oligonucleotide probes that are capable of binding specifically to genes encoding the heavy and light chains of the antibody). The encoding DNA may also be obtained by synthetic methods.

The isolated polynucleotide that encodes the bispecific or multi-specific antibodies or antigen-binding fragments thereof provided herein and/or the chimeric antigen receptors provided herein can be inserted into a vector for further cloning (amplification of the DNA) or for expression, using recombinant techniques known in the art. Many vectors are available. The vector components generally include, but are not limited to, one or more of the following: a signal sequence, an origin of replication, one or more marker genes, an enhancer element, a promoter (e.g., SV40, CMV, EF-1α), and a transcription termination sequence.

The present disclosure provides vectors comprising the isolated polynucleotides provided herein. In certain embodiments, the polynucleotides provided herein encodes the antibodies or antigen-binding fragments thereof and/or the chimeric antigen receptors provided herein, at least one promoter (e.g., SV40, CMV, EF-1α) operably linked to the nucleic acid sequence, and at least one selection marker. Examples of vectors include, but are not limited to, retrovirus (including lentivirus), adenovirus, adeno-associated virus, herpesvirus (e.g., herpes simplex virus), poxvirus, baculovirus, papillomavirus, papovavirus (e.g., SV40), lambda phage, and M13 phage, plasmid pcDNA3.3, pMD18-T, pOptivec, pCMV, pEGFP, pIRES, pQD-hyg-GSeu, pALTER, pBAD, pcDNA, pCal, pL, pET, pGEMEX, pGEX, pCI, pEGFT, pSV2, pFUSE, pVITRO, pVIVO, pMAL, pMONO, pSELECT, pUNO, pDUO, Psg5L, pBABE, pWPXL, pBI, p15TV-L, pPro18, pTD, pRS10, pLexA, pACT2.2, pCMV-SCRIPT.RTM., pCDM8, pCDNA1.1/amp, pcDNA3.1, pRc/RSV, PCR 2.1, pEF-1, pFB, pSG5, pXT1, pCDEF3, pSVSPORT, pEF-Bos etc.

Vectors comprising the polynucleotide sequence encoding the antibody or antigen-binding fragment thereof and/or the chimeric antigen receptor provided herein can be introduced to a host expression system (e.g., a host cell) for cloning or gene expression. In certain embodiments, the host expression system provided herein is a microorganism, a yeast, or a mammalian cell. In certain embodiments, the microorganism is selected from the group consisting of E. coli and B. subtilis. In certain embodiments, the yeast is Saccharomyces. In certain embodiments, the mammalian cell is selected from the group consisting of COS, CHO-S, CHO-K1, HEK-293, and 3T3 cells.

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces.

In addition to prokaryotes, eukaryotic microbes such as filamentous fungi or yeast are suitable cloning or expression hosts for the bispecific or multi-specific antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated antibodies or antigen-fragment thereof provided herein are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruiffly), and Bombyx mori have been identified. In certain embodiments, a virus comprises the vectors provided herein. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); m197ercurirtoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; mouse forestomach carcinoma cells (MFC), SNU620 cells, and a human hepatoma line (Hep G2). In some embodiments, the host cell is a mammalian cultured cell line, such as CHO, BHK, NS0, 293, MFC, SNU620 and their derivatives.

Host cells are transformed with the above-described expression or cloning vectors for the bispecific or multi-specific antibody production and cultured in conventional nutrient media modified as appropriate for inducing promoters, selecting transformants, or amplifying the genes encoding the desired sequences. In another embodiment, the antibody may be produced by homologous recombination known in the art. In certain embodiments, the host cell is capable of producing the antibody or antigen-binding fragment thereof provided herein.

The present disclosure also provides a method of expressing the antibody or an antigen-binding fragment thereof and/or the chimeric antigen receptor provided herein, comprising culturing the host expression system provided herein under the condition at which the antibody or antigen-binding fragment thereof and/or the chimeric antigen receptor is expressed. The host expression systems used to produce the antibodies or antigen-binding fragments thereof and/or chimeric antigen receptor provided herein may be cultured in a variety of media. Commercially available media such as Ham's F10 (Sigma), Minimal Essential Medium (MEM) (Sigma), RPMI-1640 (Sigma), and Dulbecco's Modified Eagle's Medium (DMEM) (Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to a person skilled in the art. The culture conditions, such as temperature, pH, and the like, are those previously used with the host cell selected for expression, and will be apparent to a person skilled in the art.

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. Carter et al., Bio/Technology 10:163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The bispecific or multi-specific antibodies or antigen-binding fragments thereof and/or the chimeric antigen receptors prepared from the host expression systems can be purified using, for example, hydroxylapatite chromatography, gel electrophoresis, dialysis, DEAE-cellulose ion exchange chromatography, ammonium sulfate precipitation, salting out, and affinity chromatography, with affinity chromatography being the preferred purification technique.

In certain embodiments, Protein A immobilized on a solid phase is used for immunoaffinity purification of the antibody and antigen-binding fragment thereof and/or the chimeric antigen receptor provided herein. The suitability of protein A as an affinity ligand depends on the species and isotype of any immunoglobulin Fc domain that is present in the antibody. Protein A can be used to purify antibodies that are based on human gamma1, gamma2, or gamma4 heavy chains (Lindmark et al., J. Immunol. Meth. 62:1-13 (1983)). Protein G is recommended for all mouse isotypes and for human gamma3 (Guss et al., EMBO J. 5:1567 1575 (1986)). The matrix to which the affinity ligand is attached is most often agarose, but other matrices are available. Mechanically stable matrices such as controlled pore glass or poly (styrenedivinyl)benzene allow for faster flow rates and shorter processing times than can be achieved with agarose. Where the antibody comprises a CH3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25M salt).

### Pharmaceutical Composition

The present disclosure further provides pharmaceutical compositions comprising the antibodies or antigen-binding fragments thereof and/or the chimeric antigen receptors provided herein and one or more pharmaceutically acceptable carriers.

Pharmaceutical acceptable carriers for use in the pharmaceutical compositions disclosed herein may include, for example, pharmaceutically acceptable liquid, gels, or solid carriers, aqueous vehicles, nonaqueous vehicles, antimicrobial agents, isotonic agents, buffers, antioxidants, anesthetics, suspending/dispending agents, sequestering or chelating agents, diluents, adjuvants, excipients, or non-toxic auxiliary substances, other components known in the art, or various combinations thereof.

Suitable components may include, for example, antioxidants, fillers, binders, disintegrants, buffers, preservatives, lubricants, flavorings, thickeners, coloring agents, emulsifiers or stabilizers such as sugars and cyclodextrins. Suitable antioxidants may include, for example, methionine, ascorbic acid, EDTA, sodium thiosulfate, platinum, catalase, citric acid, cysteine, thioglycerol, thioglycolic acid, thiosorbitol, butylated hydroxanisol, butylated hydroxytoluene, and/or propyl gallate. As disclosed herein, inclusion of one or more antioxidants such as methionine in a composition comprising an antibody or antigen-binding fragment thereof and conjugates provided herein decreases oxidation of the antibody or antigen-binding fragment thereof. This reduction in oxidation prevents or reduces loss of binding affinity, thereby improving antibody stability and maximizing shelf-life. Therefore, in certain embodiments, pharmaceutical compositions are provided that comprise one or more antibodies or antigen-binding fragments thereof as disclosed herein and one or more antioxidants such as methionine. Further provided are methods for preventing oxidation of, extending the shelf-life of, and/or improving the efficacy of an antibody or antigen-binding fragment provided herein by mixing the antibody or antigen-binding fragment with one or more antioxidants such as methionine.

To further illustrate, pharmaceutical acceptable carriers may include, for example, aqueous vehicles such as sodium chloride injection, Ringer's injection, isotonic dextrose injection, sterile water injection, or dextrose and lactated Ringer's injection, nonaqueous vehicles such as fixed oils of vegetable origin, cottonseed oil, corn oil, sesame oil, or peanut oil, antimicrobial agents at bacteriostatic or fungistatic concentrations, isotonic agents such as sodium chloride or dextrose, buffers such as phosphate or citrate buffers, antioxidants such as sodium bisulfate, local anesthetics such as procaine hydrochloride, suspending and dispersing agents such as sodium carboxymethylcelluose, hydroxypropyl methylcellulose, or polyvinylpyrrolidone, emulsifying agents such as Polysorbate 80 (TWEEN-80), sequestering or chelating agents such as EDTA (ethylenediaminetetraacetic acid) or EGTA (ethylene glycol tetraacetic acid), ethyl alcohol, polyethylene glycol, propylene glycol, sodium hydroxide, hydrochloric acid, citric acid, or lactic acid. Antimicrobial agents utilized as carriers may be added to pharmaceutical compositions in multiple-dose containers that include phenols or cres201 ercurialrials, benzyl alcohol, chlorobutanol, methyl and propyl p-hydroxybenzoic acid esters, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

The pharmaceutical compositions can be a liquid solution, suspension, emulsion, pill, capsule, tablet, sustained release formulation, or powder. Oral formulations can include standard carriers such as pharmaceutical grades of mannitol, lactose, starch, magnesium stearate, polyvinyl pyrollidone, sodium saccharine, cellulose, magnesium carbonate, etc.

In certain embodiments, the pharmaceutical compositions are formulated into an injectable composition. The injectable pharmaceutical compositions may be prepared in any conventional form, such as for example liquid solution, suspension, emulsion, or solid forms suitable for generating liquid solution, suspension, or emulsion. Preparations for injection may include sterile and/or non-pyretic solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use, and sterile and/or non-pyretic emulsions. The solutions may be either aqueous or nonaqueous.

In certain embodiments, unit-dose parenteral preparations are packaged in an ampoule, a vial or a syringe with a needle. All preparations for parenteral administration should be sterile and not pyretic, as is known and practiced in the art.

In certain embodiments, a sterile, lyophilized powder is prepared by dissolving an antibody or antigen-binding fragment as disclosed herein in a suitable solvent. The solvent may contain an excipient which improves the stability or other pharmacological components of the powder or reconstituted solution, prepared from the powder. Excipients that may be used include, but are not limited to, water, dextrose, sorbital, fructose, corn syrup, xylitol, glycerin, glucose, sucrose or other suitable agent. The solvent may contain a buffer, such as citrate, sodium or potassium phosphate or other such buffer known to a person skilled in the art at, in one embodiment, about neutral pH. Subsequent sterile filtration of the solution followed by lyophilization under standard conditions known to a person skilled in the art provides a desirable formulation. In one embodiment, the resulting solution will be apportioned into vials for lyophilization. Each vial can contain a single dosage or multiple dosages of the antibody or antigen-binding fragment thereof or composition thereof. Overfilling vials with a small amount above that needed for a dose or set of doses (e.g., about 10%) is acceptable so as to facilitate accurate sample withdrawal and accurate dosing. The lyophilized powder can be stored under appropriate conditions, such as at about 4° C. to room temperature.

Reconstitution of a lyophilized powder with water for injection provides a formulation for use in parenteral administration. In one embodiment, for reconstitution the sterile and/or non-pyretic water or other liquid suitable carrier is added to lyophilized powder. The precise amount depends upon the selected therapy being given, and can be empirically determined.

### Kits

In certain embodiments, the present disclosure provides a kit comprising the antibody or an antigen-binding fragment thereof provided herein and/or the chimeric antigen receptors provided herein and/or the pharmaceutical composition provided herein. In certain embodiments, the present disclosure provides a kit comprising the antibody or an antigen-binding fragment thereof provided herein and/or the chimeric antigen receptors provided herein and/or the pharmaceutical composition provided herein, and a second therapeutic agent. In certain embodiments, the second therapeutic agent is selected from the group consisting of a chemotherapeutic agent, an anti-cancer drug, radiation therapy agent, an immunotherapy agent, an anti-angiogenesis agent, a targeted therapy agent, a cellular therapy agent, a gene therapy agent, a hormonal therapy agent, an antiviral agent, an antibiotic, an analgesics, an antioxidant, a metal chelator, and cytokines.

Such kits can further include, if desired, one or more of various conventional pharmaceutical kit components, such as, for example, containers with one or more pharmaceutically acceptable carriers, additional containers etc., as will be readily apparent to a person skilled in the art. Instructions, either as inserts or a labels, indicating quantities of the components to be administered, guidelines for administration, and/or guidelines for mixing the components, can also be included in the kit.

### Methods of Use

The present disclosure also provides methods of treating, preventing or alleviating a disease, disorder or condition in a subject, comprising administering to the subject a therapeutically effective amount of the antibody or antigen-binding fragment thereof provided herein, and/or the pharmaceutical composition provided herein, and/or the chimeric antigen receptor provided herein. In certain embodiments, the disease, disorder or condition is a CD3 related disease, disorder or condition. In certain embodiments, the subject is human.

In some embodiments, the CD3 related disease, disorder or condition is characterized in expressing or over-expressing of CD3.

In certain embodiments, the disease, disorder or condition is immune disease, autoimmune disease (e.g., autoimmune encephalomyelitis, systemic lupus erythematosus, etc.), inflammatory disease, cancer or neurological disease. In certain embodiments, the cancer is a CD3-expressing cancer. “CD3-expressing” cancer as used herein refers to a cancer characterized in expressing CD3 protein in a cancer cell, a tumor infiltrating immune cell, or expressing CD3 in a cancer cell, a tumor infiltrating immune cell at a level significantly higher than that would have been expected of a normal cell. Various methods can be used to determine the presence and/or amount of CD3 in a test biological sample from the subject. For example, the test biological sample can be exposed to an anti-CD3 antibody or antigen-binding fragment thereof, which binds to and detects the expressed CD3 protein. Alternatively, CD3 can also be detected at nucleic acid expression level, using methods such as qPCR, reverse transcriptase PCR, microarray, SAGE, FISH, and the like. In some embodiments, the test sample is derived from a cancer cell or tissue, or tumor infiltrating immune cells. The reference sample can be a control sample obtained from a healthy or non-diseased individual, or a healthy or non-diseased sample obtained from the same individual from whom the test sample is obtained. For example, the reference sample can be a non-diseased sample adjacent to or in the neighborhood of the test sample (e.g., tumor).

In certain embodiments, the cancer is a GPRC5D-expressing cancer. “GPRC5D-expressing” cancer as used herein refers to a cancer characterized in expressing GPRC5D protein in a cancer cell, a tumor infiltrating immune cell, or expressing GPRC5D in a cancer cell, a tumor infiltrating immune cell at a level significantly higher than that would have been expected of a normal cell. Various methods can be used to determine the presence and/or amount of GPRC5D in a test biological sample from the subject. For example, the test biological sample can be exposed to an anti-GPRC5D antibody or antigen-binding fragment thereof, which binds to and detects the expressed GPRC5D protein. Alternatively, GPRC5D can also be detected at nucleic acid expression level, using methods such as qPCR, reverse transcriptase PCR, microarray, SAGE, FISH, and the like. In some embodiments, the test sample is derived from a cancer cell or tissue, or tumor infiltrating immune cells. The reference sample can be a control sample obtained from a healthy or non-diseased individual, or a healthy or non-diseased sample obtained from the same individual from whom the test sample is obtained. For example, the reference sample can be a non-diseased sample adjacent to or in the neighborhood of the test sample (e.g., tumor). In certain embodiments, the cancer is a GPRC5D-expressing B cell cancer.

In certain embodiments, the cancer is a GD2-expressing cancer. “GD2-expressing” cancer as used herein refers to a cancer characterized in expressing GD2 protein in a cancer cell, a tumor infiltrating immune cell, or expressing GD2 in a cancer cell, a tumor infiltrating immune cell at a level significantly higher than that would have been expected of a normal cell. Various methods can be used to determine the presence and/or amount of GD2 in a test biological sample from the subject. For example, the test biological sample can be exposed to an anti-GD2 antibody or antigen-binding fragment thereof, which binds to and detects the expressed GD2 protein. Alternatively, GD2 can also be detected at nucleic acid expression level, using methods such as qPCR, reverse transcriptase PCR, microarray, SAGE, FISH, and the like. In some embodiments, the test sample is derived from a cancer cell or tissue, or tumor infiltrating immune cells. The reference sample can be a control sample obtained from a healthy or non-diseased individual, or a healthy or non-diseased sample obtained from the same individual from whom the test sample is obtained. For example, the reference sample can be a non-diseased sample adjacent to or in the neighborhood of the test sample (e.g., tumor).

In certain embodiments, the cancer is a LILRB4-expressing cancer. “LILRB4-expressing” cancer as used herein refers to a cancer characterized in expressing LILRB4 protein in a cancer cell, a tumor infiltrating immune cell, or expressing LILRB4 in a cancer cell, a tumor infiltrating immune cell at a level significantly higher than that would have been expected of a normal cell. Various methods can be used to determine the presence and/or amount of LILRB4 in a test biological sample from the subject. For example, the test biological sample can be exposed to an anti-LILRB4 antibody or antigen-binding fragment thereof, which binds to and detects the expressed LILRB4 protein. Alternatively, LILRB4 can also be detected at nucleic acid expression level, using methods such as qPCR, reverse transcriptase PCR, microarray, SAGE, FISH, and the like. In some embodiments, the test sample is derived from a cancer cell or tissue, or tumor infiltrating immune cells. The reference sample can be a control sample obtained from a healthy or non-diseased individual, or a healthy or non-diseased sample obtained from the same individual from whom the test sample is obtained. For example, the reference sample can be a non-diseased sample adjacent to or in the neighborhood of the test sample (e.g., tumor).

In certain embodiments, the cancer is a CD19-expressing cancer. “CD19-expressing” cancer as used herein refers to a cancer characterized in expressing CD19 protein in a cancer cell, a tumor infiltrating immune cell, or expressing CD19 in a cancer cell, a tumor infiltrating immune cell at a level significantly higher than that would have been expected of a normal cell. Various methods can be used to determine the presence and/or amount of CD19 in a test biological sample from the subject. For example, the test biological sample can be exposed to an anti-CD19 antibody or antigen-binding fragment thereof, which binds to and detects the expressed CD19 protein. Alternatively, CD19 can also be detected at nucleic acid expression level, using methods such as qPCR, reverse transcriptase PCR, microarray, SAGE, FISH, and the like. In some embodiments, the test sample is derived from a cancer cell or tissue, or tumor infiltrating immune cells. The reference sample can be a control sample obtained from a healthy or non-diseased individual, or a healthy or non-diseased sample obtained from the same individual from whom the test sample is obtained. For example, the reference sample can be a non-diseased sample adjacent to or in the neighborhood of the test sample (e.g., tumor).

In certain embodiments, the cancer is a CD20-expressing cancer. “CD20-expressing” cancer as used herein refers to a cancer characterized in expressing CD20 protein in a cancer cell, a tumor infiltrating immune cell, or expressing CD20 in a cancer cell, a tumor infiltrating immune cell at a level significantly higher than that would have been expected of a normal cell. Various methods can be used to determine the presence and/or amount of CD20 in a test biological sample from the subject. For example, the test biological sample can be exposed to an anti-CD20 antibody or antigen-binding fragment thereof, which binds to and detects the expressed CD20 protein. Alternatively, CD20 can also be detected at nucleic acid expression level, using methods such as qPCR, reverse transcriptase PCR, microarray, SAGE, FISH, and the like. In some embodiments, the test sample is derived from a cancer cell or tissue, or tumor infiltrating immune cells. The reference sample can be a control sample obtained from a healthy or non-diseased individual, or a healthy or non-diseased sample obtained from the same individual from whom the test sample is obtained. For example, the reference sample can be a non-diseased sample adjacent to or in the neighborhood of the test sample (e.g., tumor).

In certain embodiments, the cancer is a solid tumor or hematologic tumor.

In certain embodiments, the disease, disorder or condition is selected from the group consisting of lung cancer (e.g., non-small-cell lung cancer (NSCLC), small cell lung cancer (SCLC), adenocarcinoma of the lung, or squamous cell carcinoma of the lung), peritoneal cancer, carcinoid cancer, bone cancer, pancreatic cancer, primitive neuroectodermal tumor, skin cancer, gallbladder cancer, cancer of the head or neck, squamous cell cancer, uterine cancer, ovarian cancer, rectal cancer, prostate cancer, bladder cancer (e.g., urothelial cancer), cancer of the anal region (e.g., anal squamous cell carcinoma), gastric or stomach cancer (e.g., gastrointestinal cancer), esophageal cancer, colon cancer, breast cancer, uterine cancer, liver cancer (e.g., hepatoblastoma, hepatocellular carcinoma/hepatoma, or hepatic carcinoma), cholangiocarcinoma, sarcoma, colorectal cancer, carcinoma of the fallopian tubes, salivary gland carcinoma, carcinoma of the cervix, endometrial or uterine carcinoma, osteosarcoma, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, cancer of the nasopharynx, sarcoma of soft tissue, polycythemia vera, cancer of the urethra, cancer of the penis, cancer of the kidney or ureter (e.g., rhabdoid tumor of the kidney), cutaneous T-cell lymphoma, medulloblastoma, nephroblastoma, myelodysplastic syndrome, chronic and non-chronic myeloproliferative disorder, choroid plexus papilloma, renal cell carcinoma, carcinoma of the renal pelvis, neoplasms of the central nervous system (CNS), soft tissue sarcoma (e.g., rhabdomyosarcoma, fibrosarcoma, Kaposi's sarcoma), spinal axis tumors, glioma (e.g., ependymoma, astrocytoma, anaplastic astrocytoma, oligodendroglioma, eye cancer (e.g., retinoblastoma), brain stem glioma, or mixed glioma such as oligoastrocytoma), brain tumor (e.g., glioblastoma/glioblastoma multiforme (GBM), non-glioblastoma brain tumor, or meningioma), melanoma (e.g., cutaneous or intraocular melanoma), thrombocythemia, mesothelioma, mycosis fungoides, Sezary syndrome, idiopathic myelofibrosis, solitary plasmacytoma, vestibular schwannoma, Ewing's sarcoma, chondrosarcoma, MYH associated polyposis, pituitary adenoma, pediatric cancers such as pediatric sarcomas (e.g., neuroblastoma, rhabdomyosarcoma, and osteosarcoma), hematological cancer, Hodgkin's lymphoma, non-Hodgkin's lymphoma, leukemia (e.g., lymphocytic/lymphoblastic leukemia), chronic or acute leukemia, mast cell leukemia, lymphocytic lymphomas, primary CNS lymphoma, chronic lymphocytic leukemia (CLL), acute lymphocytic leukemia (ALL), chronic myeloid leukemia (CML), acute myeloid leukemia (AML), chronic myelomonocytic leukemia (CMML), chronic lymphoblastic leukemia, acute lymphoblastic leukemia, hairy cell leukemia (HCL), Burkitt's lymphoma (BL), multiple myeloma (e.g., relapsed or refractory multiple myeloma), T or B cell lymphoma, mantle cell lymphoma (MCL) (e.g., relapsed or refractory mantle cell lymphoma), malignant melanoma, diffuse large B cell lymphoma (DLBCL), DLBCL that results from follicular lymphoma, high-grade B-cell lymphoma, primary mediastinal large B-cell lymphoma, follicular lymphoma (FL), and primary mediastinal B-cell lymphoma.

In some embodiments, the subject has been identified as having a cancer cell or tumor infiltrating immune cells expressing CD3 and/or antigens other than CD3 (e.g., GPRC5D, GD2, LILRB4, CD19 or CD20), optionally at a level significantly higher from the level normally found on non-cancer cells.

In some embodiments, the disease, disorder or condition is autoimmune disease. Exemplary autoimmune diseases include, for example, autoimmune encephalomyelitis, systemic lupus erythematosus, connective tissue disease, systemic sclerosis, Sjogren's Syndrome, rheumatoid arthritis, myasthenia gravis, neuromyelitis optica spectrum disorder, multiple sclerosis, etc.

In another aspect, methods are provided to treat, prevent or alleviate a disease, disorder or condition in a subject that would benefit from modulation of activity of CD3 and/or antigens other than CD3 (e.g., GPRC5D, GD2, LILRB4, CD19 or CD20), comprising administering to the subject a therapeutically effective amount of the antibody or antigen-binding fragment thereof provided herein, and/or the pharmaceutical composition provided herein, and/or the chimeric antigen receptor provided herein. In certain embodiments, the disease, disorder or condition is a CD3 and/or antigens other than CD3 (e.g., GPRC5D, GD2, LILRB4, CD19 or CD20) related disease, disorder or condition, which is defined above.

The therapeutically effective amount of an antibody or antigen-binding fragment provided herein will depend on various factors known in the art, such as for example body weight, age, past medical history, present medications, state of health of the subject and potential for cross-reaction, allergies, sensitivities and adverse side-effects, as well as the administration route and extent of disease development. Dosages may be proportionally reduced or increased by a person skilled in the art (e.g., physician or veterinarian) as indicated by these and other circumstances or requirements.

In certain embodiments, the antibody or antigen-binding fragment thereof and/or the chimeric antigen receptor provided herein may be administered at a therapeutically effective dosage of about 0.01 mg/kg to about 100 mg/kg. In certain embodiments, the administration dosage may change over the course of treatment. For example, in certain embodiments the initial administration dosage may be higher than subsequent administration dosages. In certain embodiments, the administration dosage may vary over the course of treatment depending on the reaction of the subject.

Dosage regimens may be adjusted to provide the optimum desired response (e.g., a therapeutic response). For example, a single dose may be administered, or several divided doses may be administered over time.

The antibodies or antigen-binding fragments thereof provided herein may be administered by any route known in the art, for example the administration is through a parenteral route comprising subcutaneous, intraperitoneal, intravenous, intramuscular, or intradermal injection; or a non-parenteral route comprising transdermal, oral, intranasal, intraocular, sublingual, rectal, or topical.

In some embodiments, the antibodies or antigen-binding fragments thereof and/or the chimeric antigen receptors provided herein may be administered alone or in combination with a therapeutically effective amount of a second therapeutic agent. For example, the antibodies or antigen-binding fragments thereof and/or the chimeric antigen receptors disclosed herein may be administered in combination with a second therapeutic agent, for example, a chemotherapeutic agent, an anti-cancer drug, a radiation therapy agent, an immunotherapy agent, a targeted therapy agent, a cellular therapy agent, a gene therapy agent, a hormonal therapy agent, an antiviral agent, an antibiotic, an analgesics, an antioxidant, a metal chelator, cytokines, an active agent, an imaging agent, a cytotoxic agent, an angiogenesis inhibitor, a kinase inhibitor, a co-stimulation molecule agonist, a co-inhibition molecule blocker, an adhesion molecule blocker, an anti-cytokine antibody or functional fragment thereof, a detectable label or reporter, an antimicrobial, a gene editing agent, a beta agonist, an viral RNA inhibitor, a polymerase inhibitor, an interferon, or a microRNA.

The term “immunotherapy” as used herein, refers to a type of therapy that stimulates immune system to fight against disease such as cancer or that boosts immune system in a general way. Examples of immunotherapy include, without limitation, checkpoint modulators, adoptive cell transfer, cytokines, oncolytic virus and therapeutic vaccines.

“Targeted therapy” is a type of therapy that acts on specific molecules associated with cancer, such as specific proteins that are present in cancer cells but not normal cells or that are more abundant in cancer cells, or the target molecules in the cancer microenvironment that contributes to cancer growth and survival. Targeted therapy targets a therapeutic agent to a tumor, thereby sparing of normal tissue from the effects of the therapeutic agent.

In certain of these embodiments, an antibody or antigen-binding fragment thereof provided herein, and/or the pharmaceutical composition provided herein, and/or the chimeric antigen receptor provided herein that is administered in combination with one or more additional therapeutic agents may be administered simultaneously with the one or more additional therapeutic agents, and in certain of these embodiments the antibody or antigen-binding fragment thereof, and/or the pharmaceutical composition provided herein, and/or the chimeric antigen receptor provided herein, and the additional therapeutic agent(s) may be administered as part of the same pharmaceutical composition. However, an antibody or antigen-binding fragment thereof, and/or a pharmaceutical composition provided herein, and/or a chimeric antigen receptor provided herein administered “in combination” with another therapeutic agent does not have to be administered simultaneously with or in the same composition as the agent. An antibody or antigen-binding fragment thereof, pharmaceutical composition, or chimeric antigen receptor administered prior to or after another agent is considered to be administered “in combination” with that agent as the phrase is used herein, even if the antibody or antigen-binding fragment, pharmaceutical composition or chimeric antigen receptor, and the second agent are administered via different routes. Where possible, additional therapeutic agents administered in combination with the antibodies or antigen-binding fragments thereof, pharmaceutical compositions, or chimeric antigen receptors disclosed herein are administered according to the schedule listed in the product information sheet of the additional therapeutic agent, or according to the Physicians' Desk Reference 2003 (Physicians' Desk Reference, 57th Ed; Medical Economics Company; ISBN: 1563634457; 57th edition (November 2002)) or protocols well known in the art.

The present disclosure further provides method of activating a T cell expressing CD3 or another target other than CD3 in vivo or in vitro, comprising contacting the T cells with the antibody or antigen-binding fragment thereof provided herein, and/or the pharmaceutical composition provided herein, and/or the chimeric antigen receptor provided herein.

The present disclosure further provides methods of modulating activity of CD3 or another target other than CD3 in a cell expressing CD3 or another target other than CD3, comprising exposing the cells to the antibodies or antigen-binding fragments thereof provided herein, and/or the pharmaceutical composition provided herein, and/or the chimeric antigen receptor provided herein.

The present disclosure further provides methods of promoting in vivo or in vitro processing of a second antigen by CD3-expressing T cells, comprising contacting the CD3-expressing T cells with the bispecific antibodies or antigen-binding fragments thereof provided herein, wherein the bispecific antibodies or antigen-binding fragments thereof are capable of specifically binding to both the CD3-expressing T cells and a second antigen thereby bringing both in close proximity.

In another aspect, the present disclosure provides methods of detecting the presence or amount of CD3 or another target other than CD3 in a sample, comprising contacting the sample with the antibody or antigen-binding fragment thereof provided herein, and/or the pharmaceutical composition provided herein, and/or the chimeric antigen receptor provided herein, and determining the presence or the amount of CD3 or another target other than CD3 in the sample.

In another aspect, the present disclosure provides a disease, disorder or condition related to CD3 or another target other than CD3 in a subject, comprising: a) contacting a sample obtained from the subject with the antibody or an antigen-binding fragment thereof provided herein and/or the pharmaceutical composition provided herein and/or the chimeric antigen receptor provided herein; b) determining the presence or amount of CD3 or another target other than CD3 in the sample; and c) correlating the presence or the amount of CD3 or another target other than CD3 to existence or status of the disease, disorder or condition in the subject.

In another aspect, the present disclosure provides kits comprising the antibody or antigen-binding fragment thereof provided herein and/or the pharmaceutical composition provided herein and/or the chimeric antigen receptor provided herein, optionally conjugated with a detectable moiety, which is useful in detecting CD3 or another target other than CD3, optionally recombinant CD3, CD3 expressed on cell surface, or CD3-expresing cells. The kits may further comprise instructions for use.

In another aspect, the present disclosure also provides use of the antibody or antigen-binding fragment thereof provided herein and/or the pharmaceutical composition provided herein and/or the chimeric antigen receptor provided herein in the manufacture of a medicament for treating, preventing or alleviating a disease, disorder or condition related to CD3 or another target other than CD3 in a subject, in the manufacture of a diagnostic reagent for diagnosing a disease, disorder or condition related to CD3 or another target other than CD3 in a subject.

The following examples are provided to better illustrate the claimed invention and are not to be interpreted as limiting the scope of the invention. All specific compositions, materials, and methods described below, in whole or in part, fall within the scope of the present invention. These specific compositions, materials, and methods are not intended to limit the invention, but merely to illustrate specific embodiments falling within the scope of the invention. A person skilled in the art may develop equivalent compositions, materials, and methods without the exercise of inventive capacity and without departing from the scope of the invention. It will be understood that many variations can be made in the procedures herein described while still remaining within the bounds of the present invention. It is the intention of the inventors that such variations are included within the scope of the invention.

### EXAMPLES

### Example 1. Chimeric 2+1 Bispecific Antibody Generation and Characterization

### 1.1 Chimeric 2+1 Bispecific Antibody Generation

Bispecific antibodies targeting GPRC5D and CD3 were generated and characterized, wherein the characterizations include binding affinity to T cells, T cell dependent cytotoxicity, T cell activation with Jurkat NFAT luciferase reporter cells, cytokines release in PBMC, and in vivo efficacy. Fab-scFv-Fab (also referred to as “chimeric 2+1 bispecific”, “humanized 2+1 bispecific” or “Fab2-scFv”) format was used for generating the bispecific antibodies. In particular, the chimeric 2+1 bispecific antibody generated herein comprises two anti-GPRC5D Fab domains and one anti-CD3 scFv domain, wherein each of the anti-GPRC5D Fab domains comprises a GPRC5D binding moiety comprising the VH and VL regions from the clone 108C4C12, and the anti-CD3 scFv domain comprises the VH and VL regions from clone 40-C12-C10-E9, wherein the VH and VL regions of clone 40-C12-C10-E9 were linked by a (GGGGS)₄ linker, and the VL region of the anti-CD3 scFv domain was linked to the N-terminal of the CH2 domain of the chimeric antibody by a (GGGGS)₂ linker, the VH region of the anti-CD3 scFv domain was linked to CH1 region of one of the anti-GPRC5D Fab domains by a (GGGGS)₂ linker. LALA mutations (L234A, L235A, according to EU numbering) were introduced to each bispecific antibody to abolish the Fc receptor binding capability. The generated chimeric 2+1 bispecific antibody comprising the Fab domain (VL region and VH region from the clone 108C4C12) targeting GPRC5D and the scFv (VL region and VH region from the clone 40-C12-C10-E9) targeting CD3 was also named as “ATG-Bis-40E9” in the present disclosure. In ATG-Bis-40E9, the Fab domain targeting GPRC5D comprises a VH region as set forth in SEQ ID NO: 1 and a VL region as set forth in SEQ ID NO: 2, the scFv targeting CD3 comprises a VH region as set forth in SEQ ID NO: 3 and a VL region as set forth in SEQ ID NO: 4. The amino acid sequences of SEQ ID NOs: 1~4 are shown in Table 14 below, and CDR sequences identified by the convention of Kabat are underlined in each sequence.

TABLE 14
Amino Acid Sequences of ATG-Bis-40E9, JNJ-64407564 and RG6234

| | | | SEQ ID | |
|---|---|---|---|---|
| Sequence Description | | | NO | Amino Acid Sequence |
| ATG- | Anti- | VH | 1 | EVQLQQSGPELVKPGASVKMSCKA |
| Bis-40E9 | GPRC5D | | | SGYTFTSYVIHWVKQKTGQGLEWI |
| | | | | GYFNPYNDGTKYNEEFKGKATLT |
| | | | | SDKSSNTAYMELNSLTSDDSAVYF |
| | | | | CARGGVRRYFDVWGAGTTVTVSS |
| | | VL | 2 | DIQMTQSPSSLSASLGERVSLTCRA |
| | | | | SQDIGSNLNWLQQEPDGTIKRLIYA |
| | | | | TFGLDSGVPKRFSGSRSGSDYSLTI |
| | | | | SSLESEDFVDYYCQQYASFPYTFG |
| | | | | GGTKLEIK |
| | Anti-CD3 | VH | 3 | EVQLQLSGPELVKPGASVKMSCKA |
| | | | | SGYTFSSYVIHWVKQKPGQGLEWI |
| | | | | GYINPYNDVIKYNEKFKGKATLTS |
| | | | | DKSSSTAYMELSSLTSEDSAVYYC |
| | | | | ARDSYYSYDGFAYWGQGTLVTVS |
| | | | | A |
| | | VL | 4 | QIVLTQSPAIMSASPGEKVTMTCSA |
| | | | | SSSVSYMHWYQQKSGTSPKRWIY |
| | | | | DTSKLASGVPARFSGSGSGTSYSLT |
| | | | | ISSMEAEDAATYYCQQWSSNPPTF |
| | | | | GGGTKVEIK |
| JNJ- | Anti- | VH | 5 | QVQLVQSGAEVKKPGASVKVSCK |
| 64407564 | GPRC5D | | | ASGYSFTGYTMNWVRQAPGQGLE |
| | | | | WMGLINPYNSDTNYAQKLQGRV |
| | | | | TMTTDTSTSTAYMELRSLRSDDTA |
| | | | | VYYCARVALRVALDYWGQGTLV |
| | | | | TVSS |
| | | VL | 6 | DIQMTQSPSSLSASVGDRVTITCKA |
| | | | | SQNVATHVGWYQQKPGKAPKRLI |
| | | | | YSASYRYSGVPSRFSGSGSGTEFTL |
| | | | | TISNLQPEDFATYYCQQYNRYPYT |
| | | | | FGQGTKLEIK |
| | Anti-CD3 | VH | 7 | EVQLVESGGGLVQPGGSLRLSCAA |
| | | | | SGFTFNTYAMNWVRQAPGKGLEW |
| | | | | VARIRSKYNNYATYYAASVKGRF |
| | | | | TISRDDSKNSLYLQMNSLKTEDTA |
| | | | | VYYCARHGNFGNSYVSWFAYWG |
| | | | | QGTLVTVSS |
| | | VL | 8 | QTVVTQEPSLTVSPGGTVTLTCRSS |
| | | | | TGAVTTSNYANWVQQKPGQAPRG |
| | | | | LIGGTNKRAPGTPARFSGSLLGGK |
| | | | | AALTLSGVQPEDEAEYYCALWYS |
| | | | | NLWVFGGGTKLTVL |
| RG6234 | Anti- | VH | 9 | EVQLVESGGGLVQPGRSLKLSCAA |
| | GPRC5D | | | SGFSFSNYGMAWVRQAATKGLEW |
| | | | | VASISTGGGNTYYRDSVKGRFIVS |
| | | | | RDNAKNTQYLQMDSLRSEDTATY |
| | | | | YCTRHDRGGLYWGQGVMVTVSS |
| | | VL | 10 | DIVMTQAPLSVSVTPGESASISCRSS |
| | | | | KSLLHSNGITYVYWYFQKPGKSPQ |
| | | | | VLIYRMSNLASGVPDRFSGSGSET |
| | | | | DFTLKISRVEAEDVGIYHCGQLLE |
| | | | | NPYTFGAGTELELK |
| | Anti-CD3 | VH | 11 | EVQLLESGGGLVQPGGSLRLSCAA |
| | | | | SGFTFSTYAMNWVRQAPGKGLEW |
| | | | | VSRIRSKYNNYATYYADSVKGRF |
| | | | | TISRDDSKNTLYLQMNSLRAEDTA |
| | | | | VYYCVRHGNFGNSYVSWFAYWG |
| | | | | QGTLVTVSS |
| | | VL | 12 | QAVVTQEPSLTVSPGGTVTLTCGSS |
| | | | | TGAVTTSNYANWVQEKPGQAFRG |
| | | | | LIGGTNKRAPGTPARFSGSLLGGK |
| | | | | AALTLSGAQPEDEAEYYCALWYSN |
| | | | | LWVFGGGTKLTVL |

JNJ-64407564 and RG6234 were used as positive controls. The construct and amino acid sequences of JNJ-64407564 and RG6234 can be obtained from WO2018017786A2 and WO2019154890A1, respectively. In particular, JNJ-64407564 comprises a GPRC5D binding moiety comprising a VH region as set forth in SEQ ID NO: 5 and a VL region as set forth in SEQ ID NO: 6, and a CD3 binding moiety comprising a VH region as set forth in SEQ ID NO: 7 and a VL region as set forth in SEQ ID NO: 8; RG6234 comprises a GPRC5D binding moiety comprising a VH region as set forth in SEQ ID NO: 9 and a VL region as set forth in SEQ ID NO: 10, and a CD3 binding moiety comprising a VH region as set forth in SEQ ID NO: 11 and a VL region as set forth in SEQ ID NO: 12. The amino acid sequences of SEQ ID NOs: 5~12 are shown in Table 14 above, and CDR sequences identified by the convention of Kabat are underlined in each sequence.

### 1.2 Chimeric 2+1 Bispecific Antibody Characterization: Binding Affinity to T Cells

To evaluate the binding affinity of the generated chimeric bispecific antibody to T cells, the isolated CD3⁺ T cells were stained by antibodies and the binding affinity was determined by FACS analysis. The protocol of FACS analysis is described as follows:
(a) CD3⁺ T cells were isolated from normal donors.
(b) The isolated CD3⁺ T cells were washed by Staining Buffer (PBS+1% BSA) in round bottom 96-well plate.
(c) Antibody dilutions were added to each well and mixed with the CD3+ T cells, incubated at 4 degrees for 1 hour.
(d) Cells were spined at 300 g for 3 minutes, then the supernatants were discarded.
(e) Cells were washed with Staining Buffer.
(f) Step (d) was repeated, and the cells were incubated with secondary antibody in Staining Buffer for 0.5 hours at 4 degrees.
(g) After incubation, step (d)-(e) were repeated.
(h) Cells were resuspended in Staining Buffer and fluorescence signals were recorded by flow cytometry.

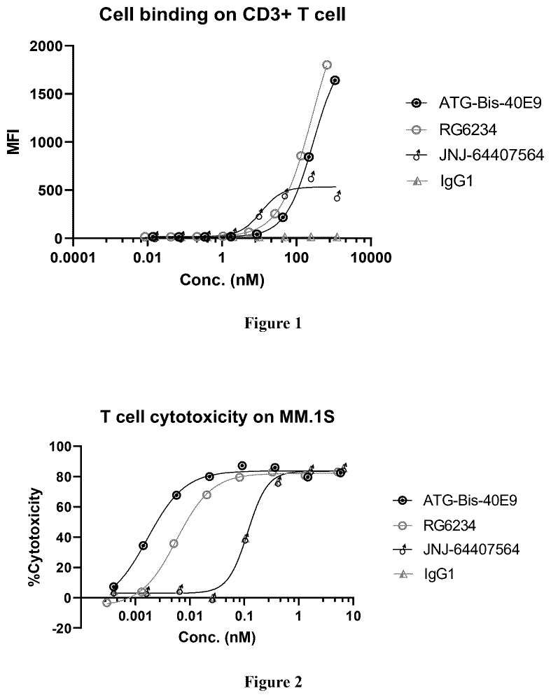

As shown in Table 1 and FIG. 1, the T cell binding affinity of ATG-Bis-40E9 showed almost identical EC₅₀ and lower top MFI compared with the benchmark antibody RG6234. On the other hand, ATG-Bis-40E9 showed higher EC₅₀ and top MFI compared with the other benchmark antibody JNJ-64407564, indicating that the T cell killing effect of ATG-Bis-40E9 was more potent than that of JNJ-64407564.

TABLE 1
Top MFI and EC₅₀ binding affinity of ATG-Bis-40E9 on T cells

| Antibody | Top (MFI) | EC₅₀ (nM) |
|---|---|---|
| ATG-Bis-40E9 | 1990 | 287.3 |
| RG6234 | 2553 | 273.7 |
| JNJ-64407564 | 534 | 11.39 |
| IgG1 | 124 | Unstable |

### 1.3 Chimeric 2+1 Bispecific Antibody Characterization: T-Cell Dependent Cytotoxicity

The cytotoxicity of chimeric 2+1 bispecific antibodies on different multiple myeloma cell lines (MM.1S, MOLP-8, L363, RPMI8226) cells was tested by FACS analysis. The protocol for FACS analysis is described as follows:
(a) CD3⁺ T cells were isolated from normal donors.
(b) 2×10⁴ or 1×10⁴ target cells/well (CFSE-labeled MM.1S, MOLP-8, L363, RPMI8226 and Daudi) and 1×10⁵ effector cells/well (T cells) were added to wells of 96-well U-bottom plates (5:1 or 10:1 Effector: Target ratio).
(c) After mixing the target cells and T cells, dilutions of chimeric 2+1 bispecific antibody were added to each well (9 diluting concentrations from 5 nM, by 1:4 serial).
(d) The plates were incubated at 37° C. with 5% CO₂ overnight.
(e) After incubation, the plates were centrifuged and analyzed by FACS.
(f) The supernatant was analyzed with IL-2 (BD Biosciences, cat #555142), IFNγ (BD Biosciences, cat #353377) ELISA preformed according to the manufacturer's instructions.

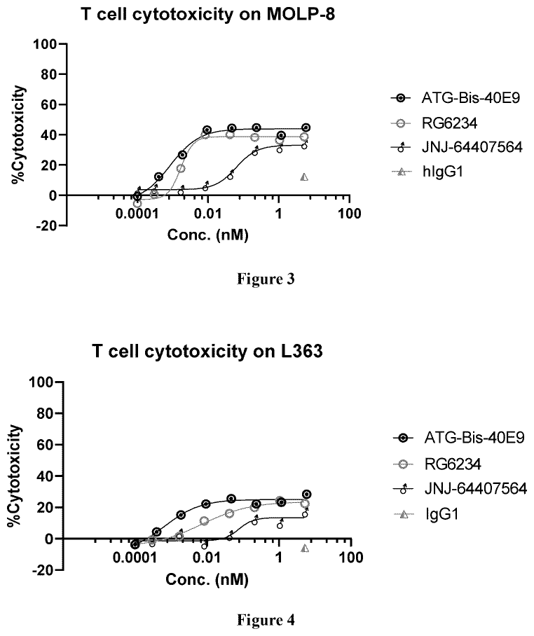

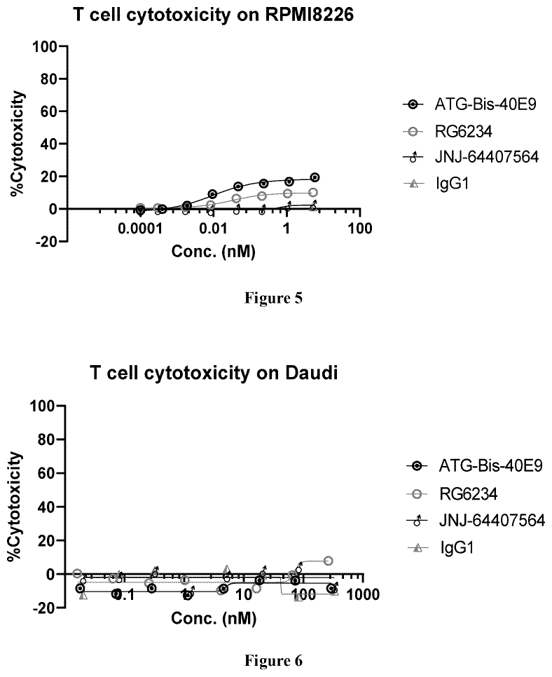

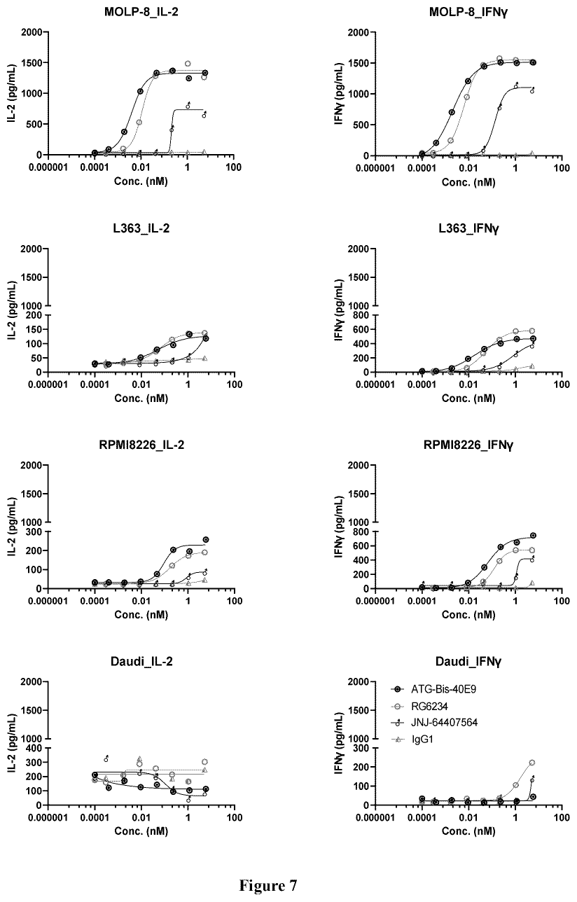

As shown in Table 2 and FIGS. 2-7, ATG-Bis-40E9 showed potent T cell dependent cytotoxicity on MM.1S, MOLP-8, L363, RPMI8226, and had lower IC50 and higher top cytotoxicity percent compared with benchmark antibodies (JNJ-64407564 and RG6234). In addition, on GPRC5D negative expressed Daudi cells, ATG-Bis-40E9 had no cytotoxicity. In terms of cytokines release, ATG-Bis-40E9 induced higher IL-2 and IFNγ release in MOLP-8, L363 and RPMI8226 cells, but induced less IL-2 and IFNγ release in Daudi cells compared with benchmark antibodies RG6234 and JNJ-64407564. These data showed that ATG-Bis-40E9 had better T cell cytotoxicity and safety compared with the benchmark antibodies.

TABLE 2
Top target cells killing percentage and IC₅₀ of ATG-Bis-40E9
induced by T-Cell Dependent Cytotoxicity

| | MM.1S | | MOLP-8 | | L363 | | RPMI8226 | | Daudi | |
|---|---|---|---|---|---|---|---|---|---|---|
| | Top | IC₅₀ | Top | IC₅₀ | Top | IC₅₀ | Top | IC₅₀ | Top | IC₅₀ |
| | (%) | (nM) | (%) | (nM) | (%) | (nM) | (%) | (nM) | (%) | (nM) |
| ATG-Bis-40E9 | 83.82 | 0.002 | 43.94 | 0.001 | 25.07 | 0.001 | 18.27 | 0.009 | −5.333 | Unstable |
| RG6234 | 82.18 | 0.006 | 38.79 | 0.002 | 23.65 | 0.007 | 9.797 | 0.031 | 7.8 | Unstable |
| JNJ-64407564 | 83.62 | 0.116 | 33.14 | 0.063 | 13.44 | 0.077 | 2.426 | 0.705 | Unstable | Unstable |

1.4 Chimeric 2+1 Bispecific Antibody Characterization: T Cell Activation with Jurkat NEAT Reporter Cell Line

To characterize the T cell activation by ATG-Bis-40E9, a Jurkat NFAT reporter cell line was employed, wherein the NFAT response elements driven luciferase gene will be induced to express upon T cell activation. Different multiple myeloma cell lines (MM.1S, MOLP-8, L363, RPMI8226) and GPRC5D negative expressed Daudi cells were used as target cells and Jurkat was used as effector cells. The protocol is described as follows:
(a) Target and effector cells were harvested and re-suspended to be 2.5×10⁴ cells/well and 5×10⁴ cells/well, respectively.
(b) 10 μg/ml or 1 μg/ml antibodies were added to cells, incubated at 37° C., 5% CO₂ for 24 hours.
(c) Cells were centrifuged at 500 g for 10 minutes, and then the supernatants were discarded.
(d) 30 μL 1× lysis buffer (Promega RLB-E3971) was added to wells and incubated at −80° C. for 30 minutes.
(e) The plates were transferred into 37° C. incubator and incubated for 15 minutes to lysis cells. The lysates were spined at 500 g for 10 minutes and the supernatants were harvested for the test.
(f) 20 μl supernatant was transferred into 96-well white opaque plate, then mixed thoroughly with 100 μl Luciferase Assay Substrate. Luciferase signal was read by plate reader.

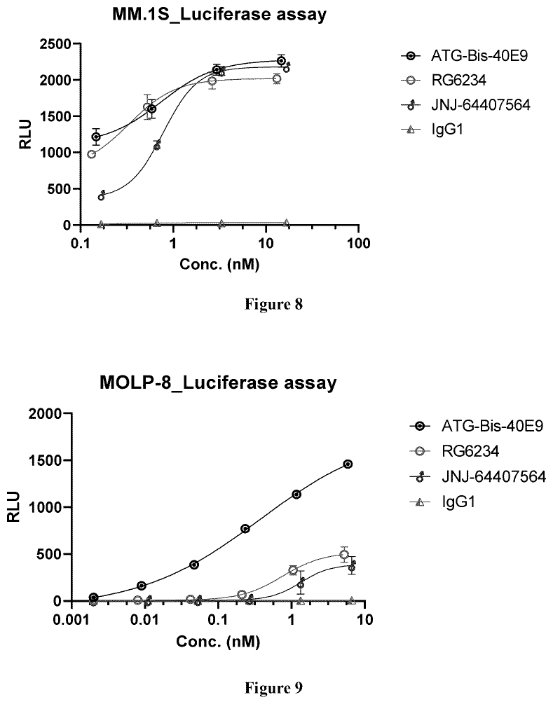

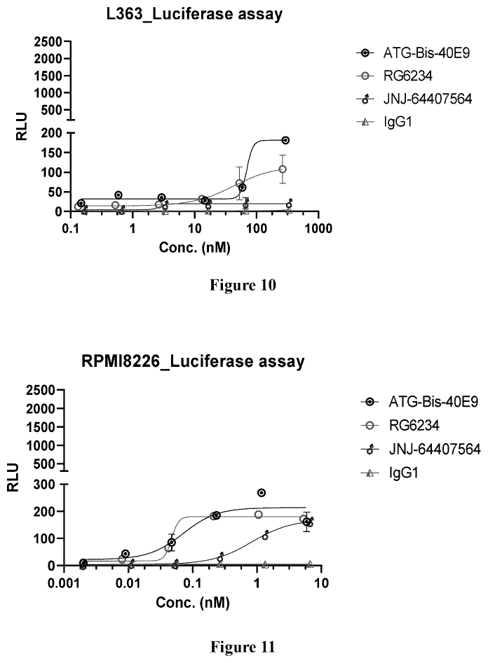

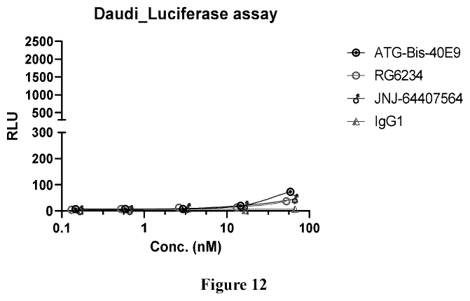

As shown in Table 3 and FIGS. 8-12, the ATG-Bis-40E9 showed potent T cell activation on different multiple myeloma cell lines and higher top RLU values compared with benchmark antibodies JNJ-64407564 and RG6234.

TABLE 3
Top RLU and EC₅₀ of ATG-Bis-40E9 induced by luciferase assay

| | MM.1S | | MOLP-8 | | L363 | | RPMI8226 | | Daudi | |
|---|---|---|---|---|---|---|---|---|---|---|
| | Top | EC₅₀ | Top | EC₅₀ | Top | EC₅₀ | Top | EC₅₀ | Top | EC₅₀ |
| | (RLU) | (nM) | (RLU) | (nM) | (RLU) | (nM) | (RLU) | (nM) | (RLU) | (nM) |
| ATG-Bis- | 2279 | 0.739 | 1813 | 0.374 | 181 | 69.39 | 213.6 | 0.071 | Unsta- | Unsta- |
| 40E9 | | | | | | | | | ble | ble |
| RG6234 | 2018 | 0.334 | 518.5 | 0.756 | 116.9 | 44.04 | 180.5 | 0.047 | Unsta- | Unsta- |
| | | | | | | | | | ble | ble |
| JNJ- | 2180 | 0.78 | 390.8 | 1.35 | 20.17 | 2.829 | 169.7 | 0.832 | Unsta- | Unsta- |
| 64407564 | | | | | | | | | ble | ble |
| IgG1 | 30.68 | Unsta- | Unsta- | Unsta- | 23.43 | Unsta- | 5.33 | Unsta- | 5.5 | Unsta- |
| | | ble | ble | ble | | ble | | ble | | ble |

### 1.5 Chimeric 2+1 Bispecific Antibody Characterization: Cytokines Release in PBMC

The biosafety of ATG-Bis-40E9 was evaluated by cytokines release of PBMC. The protocol is described as follows:
(a) Day 1: antibodies were coated on 96 well plate at 4° C. overnight. The final concentrations were at 6 dose points, from 588 nM with 10-fold series dilution.
(b) Day 2:0.1 million cells/well fresh PBMC were seeded in 96 well plate. Cells were treated with either plate bound antibodies or soluble antibodies (the final concentration at 6 dose points, from 2940 nM with 10-fold series dilution).
(c) Day 4: After 48 hours incubation, the supernatant was analyzed with IL-2 (BD Biosciences, cat #555142), IFNγ (BD Biosciences, cat #353377) and IL-6 (BD Biosciences, cat #550799) ELISA preformed according to the manufacturer's instructions.

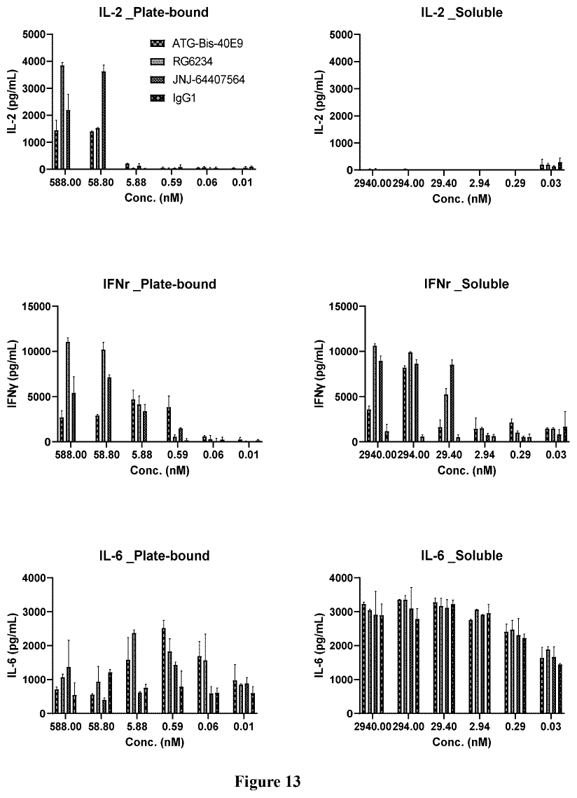

As shown in FIG. 13, compared to the benchmark antibodies (JNJ-64407564 and RG6234), ATG-Bis-40E9 induced less IL-2, IFNγ and IL-6 release from PBMC with both plate-bound and soluble antibody at top concentration. Therefore, ATG-Bis-40E9 showed potent biosafety in PBMC activation compared with benchmark antibodies JNJ-64407564 and RG6234.

1.6 Chimeric 2+1 Bispecific Antibody Characterization: In vivo Efficacy

To assess the activity of chimeric 2+1 bispecific antibody in vivo, human PBMCs humanized MM.1S model was used. Each NCG (NOD CRISPR Prkdc Il2r gamma) mouse received 1×10⁷ MM.1S human MM (multiple myeloma) cells implanted SC (subcutaneous injection) in the right flank. After 7 days, the animals received an I. V. injection of 1×10⁷ human PBMCs. Mice were randomized to groups with similar starting tumor volume when tumor volume reached 70-80 mm³, on the same day, each mouse received I.V. administration of test samples. Treatments were administered in one dose per three days. JNJ-64407564 was used as the benchmark antibody. Tumor growth inhibition mean was calculated by using the equation below, wherein Mean (Control) indicates the tumor volume mean of the mice treated with hIgG isotype control; Mean (Control DO) indicates the tumor volume mean of the mice treated with hIgG isotype control at Day 0; Mean (Test) indicates the tumor volume mean of the mice treated with the tested bispecific antibodies; Mean (Test DO) indicates the tumor volume mean of the mice treated with the tested bispecific antibodies at Day 0.

$$Mean \% Δ Inhibition = \frac{( Mean ( Control ) - Mean ( Control D0 ) ) - ( Mean ( Test ) - Mean ( Test D0 ) )}{Mean ( Control ) - Mean ( Control D0 )} \times 100 \%$$

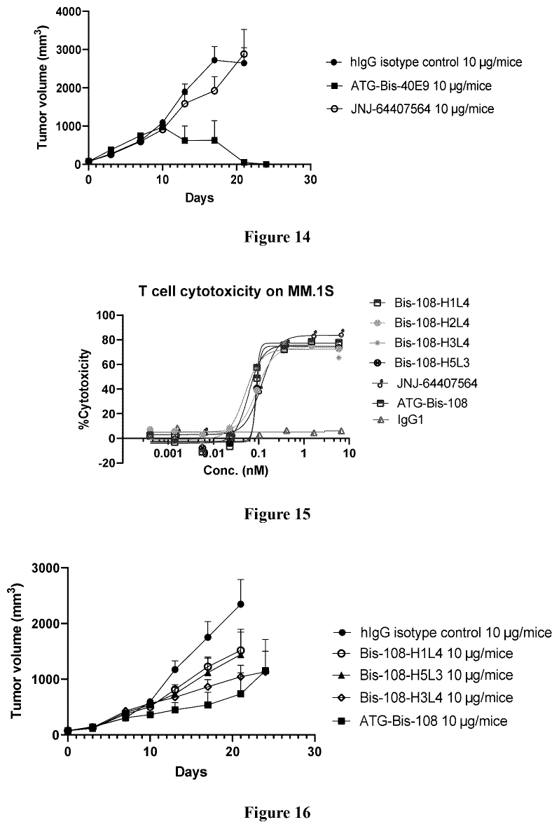

As shown in Table 4 and FIG. 14, ATG-Bis-40E9 exhibited more potent in vivo anti-tumor efficacy than JNJ-64407564, suggesting ATG-Bis-40E9's great in vivo therapeutic potential.

TABLE 4
Tumor growth inhibition mean of ATG-Bis-40E9

| hIgG isotype | Clone No. | | | | | | |
|---|---|---|---|---|---|---|---|
| control | D0 | D3 | D7 | D10 | D13 | D17 | D21 |

| | | | | | | |
|---|---|---|---|---|---|---|
| ATG-Bis- | −54.50% | −26.34% | 12.41% | 70.11% | 79.37% | 101.06% |
| 40E9 | | | | | | |
| JNJ- | 7.79% | 3.07% | 18.23% | 17.23% | 30.23% | −9.18% |
| 64407564 | | | | | | |

### Example 2. Humanized 2+1 Bispecific Antibody Generation and Characterization

### 2.1 Humanized 2+1 Bispecific Antibody Generation

Bispecific antibodies targeting GPRC5D and CD3 were constructed by using the Fab-scFv-Fab format. In particular, each of the bispecific antibodies generated herein comprises two humanized anti-GPRC5D Fab domains and one anti-CD3 scFv domain, wherein each of the humanized anti-GPRC5D Fab domains comprises a GPRC5D binding moiety comprising a humanized VH region and a humanized VL region from the clone 108C4C12, and the anti-CD3 scFv domain comprises the VH and VL regions from the anti-CD3 domain of benchmark antibody JNJ-64407564, wherein the VH region of the anti-CD3 scFv domain comprises an amino acid sequence as set forth in SEQ ID NO:7, and the VL region of the anti-CD3 scFv domain comprises an amino acid sequence as set forth in SEQ ID NO:8, wherein the VH and VL regions of the anti-CD3 scFv domain were linked by a (GGGGS)₄ linker, and the VL region of the anti-CD3 scFv domain was linked to the N-terminal of the CH2 domain of the generated antibody by a (GGGGS)₂ linker, the VH region of the anti-CD3 scFv domain was linked to CH1 region of one of the anti-GPRC5D Fab domains by a (GGGGS)₂ linker. LALA mutations (L234A, L235A, according to EU numbering) were introduced to each bispecific antibody to abolish the Fc receptor binding capability. The resulting bispecific antibodies are referred to herein as Bis-108-H1L4, Bis-108-H2L4, Bis-108-H3L4, Bis-108-H5L3, where the prefix “Bis-” indicates “bispecific”, and the following indicates the humanized antibody from which the clone 108C4C12 was derived. For example, “Bis-108-HIL4” indicates that it is a bispecific antibody with a Fab-scFv-Fab format, comprising a Fab domain targeting GPRC5D (which comprises a humanized VH1 region and a humanized VL4 region from the clone 108C4C12) and a scFv targeting CD3 (which comprises a VH region comprising an amino acid sequence as set forth in SEQ ID NO: 7 and a VL region comprising an amino acid sequence as set forth in SEQ ID NO: 8). The humanized VH1 region from the clone 108C4C12 comprises an amino acid sequence as set forth in SEQ ID NO:13, the humanized VH2 region from the clone 108C4C12 comprises an amino acid sequence as set forth in SEQ ID NO: 14, the humanized VH3 region from the clone 108C4C12 comprises an amino acid sequence as set forth in SEQ ID NO:15, the humanized VH5 region from the clone 108C4C12 comprises an amino acid sequence as set forth in SEQ ID NO: 16, the humanized VL3 region from the clone 108C4C12 comprises an amino acid sequence as set forth in SEQ ID NO: 17, and the humanized VL4 region from the clone 108C4C12 comprises an amino acid sequence as set forth in SEQ ID NO: 18. The amino acid sequences of SEQ ID NOs: 13~18 are shown in Table 15 below, and CDR sequences identified by the convention of Kabat are underlined in each sequence.

TABLE 15
Amino Acid Sequences of Humanized VH and VL Regions of Clone

| Sequence | | SEQ ID | |
|---|---|---|---|
| Description | | NO | Amino Acid Sequence |
| 108C4C12 | VH1 | 13 | EVQLVQSGAEVKKPGASVKVSCKASGYT |
| | | | FTSYVIHWVRQAPGQGLEWMGYFNPYN |
| | | | DGTKYNEEFKGRVTMTTDTSTSTAYMEL |
| | | | RSLRSDDTAVYYCARGGVRRYFDVWGQ |
| | | | GTTVTVSS |
| | VH2 | 14 | EVQLVQSGAEVKKPGASVKVSCKASGYT |
| | | | FTSYVIHWVRQAPGQGLEWIGYFNPYND |
| | | | GTKYNEEFKGRATMTTDTSTSTAYMELR |
| | | | SLRSDDTAVYYCARGGVRRYFDVWGQG |
| | | | TTVTVSS |
| | VH3 | 15 | EVQLVQSGAEVKKPGASVKVSCKASGYT |
| | | | FTSYVIHWVRQAPGQGLEWMGYFNPYN |
| | | | DGTKYNEEFKGRVTLTSDKSTSTAYMEL |
| | | | RSLRSDDTAVYYCARGGVRRYFDVWGQ |
| | | | GTTVTVSS |
| | VH5 | 16 | EVQLVQSGAEVKKPGASVKVSCKASGYT |
| | | | FTSYVIHWVKQAPGQGLEWIGYFNPYND |
| | | | GTKYNEEFKGKATLTTDKSTSTAYMELR |
| | | | SLRSDDTAVYYCARGGVRRYFDVWGQG |
| | | | TTVTVSS |
| | VL3 | 17 | DIQMTQSPSSLSASVGDRVTITCRASQDIG |
| | | | SNLNWLQQKPGKAIKRLIYATFGLDSGVP |
| | | | SRFSGSGSGTDYTLTISSLQPEDFATYYCQ |
| | | | QYASFPYTFGGGTKVEIK |
| | VL4 | 18 | DIQMTQSPSSLSASVGDRVTITCRASQDIG |
| | | | SNLNWLQQKPGKAIKRLIYATFGLDSGVP |
| | | | SRFSGSRSGTDFTLTISSLQPEDFATYYCQ |
| | | | QYASFPYTFGGGTKVEIK |

ATG-Bis-108 was used as the parental antibody control of these generated bispecific antibodies. ATG-Bis-108 had the same construct and amino acid sequences as ATG-Bis-40E9, except that the scFv targeting CD3 used in ATG-Bis-108 comprises a VH region as set forth in SEQ ID NO: 7 and a VL region as set forth in SEQ ID NO: 8.

### 2.2 Humanized 2+1 Bispecific Antibody Characterization: T-Cell Dependent Cytotoxicity

The cytotoxicity of the generated humanized GPRC5D bispecific antibodies on MM.1S cells was tested by FACS analysis. The protocol for FACS analysis is described as follows:
(a) CD3⁺ T cells were isolated from normal donors.
(b) 2×10⁴ target cells/well (CFSE-labeled MM.1S) and 1×10⁵ effector cells/well (T cells) were added to wells of 96-well U-bottom plates (5:1 Effector: Target ratio).
(c) After mixing the target cells and T cells, initial concentration of 5 nM with 9 data points by 1:4 serial dilution of the generated 2+1 bispecific antibodies were added to each well.
(d) The plates were incubated at 37° C. with 5% CO₂ overnight.
(e) After incubation, the plates were centrifuged and analyzed by FACS.
(f) The supernatant was analyzed by IL-2 (BD Biosciences, cat #555142), IFNγ (BD Biosciences, cat #353377) ELISA preformed according to the manufacturer's instructions.

As shown in Table 5 and FIG. 15, the humanized GPRC5D bispecific antibodies generated herein showed potent T cell dependent cytotoxicity on MM.1S cells and had lower IC₅₀ than JNJ-64407564. Among these generated humanized GPRC5D bispecific antibodies, Bis-108-H3L4 had the most potent T cell cytotoxicity (IC₅₀).

TABLE 5
Top target cells killing percentage and IC₅₀ of humanized GPRC5D
bispecific antibody induced by T-Cell Dependent Cytotoxicity

| | IC₅₀ | Top |
|---|---|---|
| Antibody | (nM) | (%) |
| Bis-108-H3L4 | 0.04965 | 72.43 |
| Bis-108-H1L4 | 0.05971 | 75.55 |
| Bis-108-H5L3 | 0.0897 | 74.77 |
| Bis-108-H2L4 | 0.09339 | 73.99 |
| ATG-Bis-108 | 0.08705 | 77.23 |
| JNJ-64407564 | 0.116 | 83.62 |
| IgG1 | Unstable | 6.079 |

2.3 Humanized 2+1 Bispecific Antibody Characterization: In vivo Efficacy

To assess the activity of the generated humanized GPCR5D bispecific antibodies in vivo, human PBMCs humanized MM.1S model was used, which was generated by using a similar protocol as described in Example 1.6, except that different test antibodies were used. ATG-Bis-108 was used as the parental antibody control of these humanized GPRC5D bispecific antibodies. The tumor growth inhibition mean was calculated by using the same equation as described in Example 1.6.

As shown in Table 6 and FIG. 16, all of the generated humanized GPRC5D bispecific antibodies exhibited potent in vivo anti-tumor efficacy, and Bis-108-H3L4 showed more potent in vivo anti-tumor efficacy than other humanized GPRC5D bispecific antibodies.

TABLE 6
Tumor growth inhibition mean of humanized GPRC5D bispecific antibodies

| hIgG isotype | Clone No. | | | | | | |
|---|---|---|---|---|---|---|---|
| control | D0 | D2 | D5 | D9 | D12 | D16 | D19 |

| | | | | | | |
|---|---|---|---|---|---|---|
| Bis-108-H1L4 | 28.69% | −31.83% | 17.78% | 32.50% | 31.58% | 36.56% |
| Bis-108-H3L4 | 19.92% | −55.92% | 6.36% | 45.15% | 52.97% | 57.43% |
| Bis-108-H5L3 | 42.26% | −43.82% | 11.08% | 39.46% | 37.77% | 39.73% |
| ATG-Bis-108 | 7.76% | −3.07% | 44.74% | 65.98% | 72.38% | 70.83% |

### Example 3. Humanized 2+1 Bispecific Antibody Generation and Characterization

### 3.1 Humanized 2+1 Bispecific Antibody Generation

Bispecific antibodies targeting GPRC5D and CD3 were generated and characterized, wherein the characterizations include binding affinity to T cells, T cell dependent cytotoxicity, T cell activation with Jurkat NFAT luciferase reporter cells, cytokines release in PBMC, and in vivo efficacy. Fab-scFv-Fab format was used in generating the bispecific antibodies. In particular, each of the humanized Fab-scFv-Fab bispecific antibodies generated herein comprises two humanized anti-GPRC5D Fab domains and one humanized and affinity matured anti-CD3 scFv domain, wherein each of the humanized anti-GPRC5D Fab domains comprises a GPRC5D binding moiety comprising a humanized VH region and a humanized VL region from the clone 108C4C12, and the humanized and affinity matured anti-CD3 scFv domain comprises a humanized and affinity matured VH region and a humanized and affinity matured VL region from the clone 40-C12-C10-E9, wherein the humanized VH region of each of the anti-GPRC5D Fab domains comprises an amino acid sequence as set forth in SEQ ID NO: 15, and the humanized VL region of each of the anti-GPRC5D Fab domains comprises an amino acid sequence as set forth in SEQ ID NO: 18, wherein the VH and VL regions of the humanized and affinity matured anti-CD3 scFv domain were linked by a (GGGGS)₄ linker, and the VL region of the humanized and affinity matured anti-CD3 scFv domain was linked to the N-terminal of the CH2 domain of the generated antibody by a (GGGGS)₂ linker, the VH region of the humanized and affinity matured anti-CD3 scFv domain was linked to CH1 domain of one of the anti-GPRC5D Fab domains by a (GGGGS)₂ linker. LALA mutations (L234A, L235A, according to EU numbering) were introduced to each bispecific antibody to abolish the Fc receptor binding capability. The resulting humanized 2+1 bispecific antibodies are referred to herein as Bis-40E9-L2H1, Bis-40E9-L2H3, Bis-40E9-L2H4, Bis-40E9-L3H1, Bis-40E9-L2H3-D99E.H, Bis-40E9-L2H3-Y101F.H, Bis-40E9-L2H3-G106A.H, Bis-40E9-L2H3-Y54G.H, Bis-40E9-L2H3-D105R.H, wherein the prefix “Bis-” indicates “bispecific”, and the following indicates the humanized antibody from which the clone 40-C12-C10-E9 was derived. For example, “Bis-40E9-L2H1” indicates that it is a bispecific antibody with a Fab-scFv-Fab format, comprising a scFv (which comprises a humanized VL2 region and a humanized VH1 region from the hybridoma antibody clone 40-C12-C10-E9) targeting CD3 and a humanized Fab domain targeting GPRC5D (which comprises a VH region comprising an amino acid sequence as set forth in SEQ ID NO: 15 and a VL region comprising an amino acid sequence as set forth in SEQ ID NO: 18). The SEQ ID NOs and amino acid sequences of VL and VH regions of the scFv targeting CD3 in each of the generated humanized 2+1 bispecific antibodies (i.e., Bis-40E9-L2H1, Bis-40E9-L2H3, Bis-40E9-L2H4, Bis-40E9-L3H1, Bis-40E9-L2H3-D99E.H, Bis-40E9-L2H3-Y101F.H, Bis-40E9-L2H3-G106A.H, Bis-40E9-L2H3-Y54G.H, Bis-40E9-L2H3-D105R.H) are shown in Table 16, Table 17 and Table 18 below, and CDR sequences identified by the convention of Kabat are underlined in each sequence listed in Table 18.

TABLE 16
VH and VL Regions of the anti-CD3 scFv Domain of
the Generated Exemplary Bispecific Antibodies

| | VL Region | |
|---|---|---|
| | 40E9-VL2 | 40E9-VL3 |
| VH Region | (SEQ ID NO: 198) | (SEQ ID NO: 199) |
| 40E9-VH1 | 40E9-L2H1 | 40E9-L3H1 |
| (SEQ ID NO: 195) | (SEQ ID NOs: 198/195) | (SEQ ID NOs: 199/195) |
| 40E9-VH3 | 40E9-L2H3 | / |
| (SEQ ID NO: 196) | (SEQ ID NOs: 198/196) | |
| 40E9-VH4 | 40E9-L2H4 | / |
| (SEQ ID NO: 197) | (SEQ ID NOs: 198/197) | |

TABLE 17
VH and VL Regions of the anti-CD3 scFv Domain of
the Generated Exemplary Bispecific Antibodies

| | VL Region |
|---|---|
| | 40E9-VL2 |
| VH Region | (SEQ ID NO: 198) |
| 40E9-VH3-D99E.H | 40E9-L2H3-D99E.H |
| (SEQ ID NO: 200) | (SEQ ID NOs: 198/200) |
| 40E9-VH3-Y101F.H | 40E9-L2H3-Y101F.H |
| (SEQ ID NO: 201) | (SEQ ID NOs: 198/201) |
| 40E9-VH3-G106A.H | 40E9-L2H3-G106A.H |
| (SEQ ID NO: 202) | (SEQ ID NOs: 198/202) |
| 40E9-VH3-Y54G.H | 40E9-L2H3-Y54G.H |
| (SEQ ID NO: 203) | (SEQ ID NOs: 198/203) |
| 40E9-VH3-D105R.H | 40E9-L2H3-D105R.H |
| (SEQ ID NO: 204) | (SEQ ID NOs: 198/204) |

TABLE 18
Amino Acid Sequences of VH and VL Regions of Humanized 40-C12-
C10-E9

| Description | SEQ ID NO: | Amino Acid Sequence |
|---|---|---|
| 40E9-VH1 | 195 | EVQLVQSGAEVKKPGSSVKVSCKASGGTFSSYVI |
| | | HWVRQAPGQCLEWMGYINPYNDVIKYNEKFK |
| | | GRVTITADKSTSTAYMELSSLRSEDTAVYYCAR |
| | | DSYYSYDGFAYWGQGTLVTVSS |
| 40E9-VH3 | 196 | EVQLVQSGAEVKKPGSSVKVSCKASGYTFSSYVI |
| | | HWVRQAPGQCLEWMGYINPYNDVIKYNEKFK |
| | | GRVTLTSDKSTSTAYMELSSLRSEDTAVYYCAR |
| | | DSYYSYDGFAYWGQGTLVTVSS |
| 40E9-VH4 | 197 | EVQLVQSGAEVKKPGSSVKVSCKASGYTFSSYVI |
| | | HWVRQAPGQCLEWIGYINPYNDVIKYNEKFKG |
| | | RATLTSDKSTSTAYMELSSLRSEDTAVYYCARDS |
| | | YYSYDGFAYWGQGTLVTVSS |
| 40E9-VL2 | 198 | EIVLTQSPDFQSVTPKEKVTITCSASSSVSYMHW |
| | | YQQKPDQSPKRLIYDTSKLASGVPSRFSGSGSGT |
| | | DFTLTINSLEAEDAATYYCQQWSSNPPTFGCGT |
| | | KVEIK |
| 40E9-VL3 | 199 | EIVLTQSPDFQSVTPKEKVTITCSASSSVSYMHW |
| | | YQQKPDQSPKRWIYDTSKLASGVPSRFSGSGSG |
| | | TDYTLTINSLEAEDAATYYCQQWSSNPPTFGCG |
| | | TKVEIK |
| 40E9-VH3- | 200 | EVQLVQSGAEVKKPGSSVKVSCKASGYTFSSYVI |
| D99E.H | | HWVRQAPGQCLEWMGYINPYNDVIKYNEKFK |
| | | GRVTLTSDKSTSTAYMELSSLRSEDTAVYYCAR |
| | | ESYYSYDGFAYWGQGTLVTVSS |
| 40E9-VH3- | 201 | EVQLVQSGAEVKKPGSSVKVSCKASGYTFSSYVI |
| Y101F.H | | HWVRQAPGQCLEWMGYINPYNDVIKYNEKFK |
| | | GRVTLTSDKSTSTAYMELSSLRSEDTAVYYCAR |
| | | DSFYSYDGFAYWGQGTLVTVSS |
| 40E9-VH3- | 202 | EVQLVQSGAEVKKPGSSVKVSCKASGYTFSSYVI |
| G106A.H | | HWVRQAPGQCLEWMGYINPYNDVIKYNEKFK |
| | | GRVTLTSDKSTSTAYMELSSLRSEDTAVYYCAR |
| | | DSYYSYDAFAYWGQGTLVTVSS |
| 40E9-VH3- | 203 | EVQLVQSGAEVKKPGSSVKVSCKASGYTFSSYVI |
| Y54G.H | | HWVRQAPGQCLEWMGYINPGNDVIKYNEKFK |
| | | GRVTLTSDKSTSTAYMELSSLRSEDTAVYYCAR |
| | | DSYYSYDGFAYWGQGTLVTVSS |
| 40E9-VH3- | 204 | EVQLVQSGAEVKKPGSSVKVSCKASGYTFSSYVI |
| D105R.H | | HWVRQAPGQCLEWMGYINPYNDVIKYNEKFK |
| | | GRVTLTSDKSTSTAYMELSSLRSEDTAVYYCAR |
| | | DSYYSYRGFAYWGQGTLVTVSS |

ATG-Bis-40E9 described in Example 1 was used as the parental antibody control of these antibodies. JNJ-64407564 and RG6234 were used as positive controls, and hIgG1 isotype was used as negative control.

### 3.2 Humanized 2+1 Bispecific Antibody Characterization: Binding Affinity to Jurkat Cells

The binding affinities of the generated humanized Fab-scFv-Fab bispecific antibodies were determined by FACS analysis. The protocol for FACS analysis is described as follows:
(a) Jurkat cells were washed by Staining Buffer (PBS+1% BSA) in round bottom 96-well plate.
(b) Antibody dilutions and cells were added to each well, incubated at 4 degrees for 1 hour.
(c) Cells were spined down at 300 g for 3 minutes, and the supernatants were discarded.
(d) Cells were washed with Staining Buffer.
(e) Step (d) was repeated and the cells were incubated for 0.5 hours at 4 degrees with secondary antibody in Staining Buffer.
(f) After incubation, step (d)-(e) were repeated.
(g) Staining Buffer was added and fluorescence signals were recorded by flow cytometry.

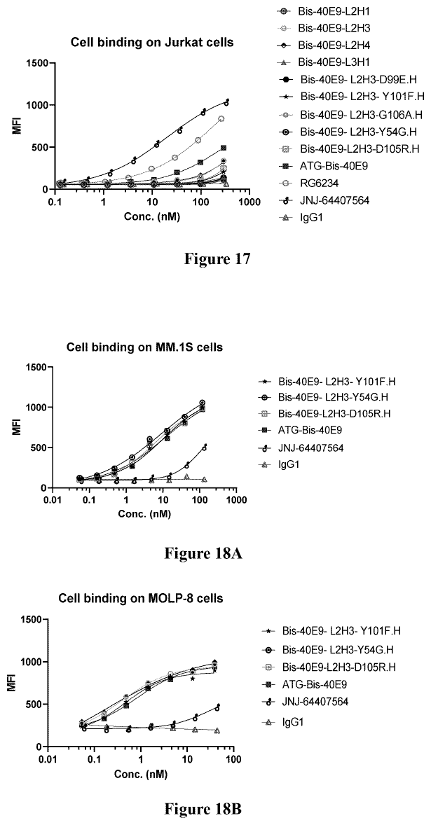

As shown in Table 7 and FIG. 17, the generated humanized Fab-scFv-Fab bispecific antibodies showed binding ability to Jurkat cells and more potent T cell killing effect compared with benchmark antibodies, JNJ-64407564 and RG6234.

TABLE 7
MFI of Jurkat cell binding affinity of humanized 2+1
bispecific antibodies

| | MFI |
|---|---|
| Antibody | (50 μg/ml) |
| Bis-40E9-L2H3 | 341 |
| Bis-40E9-L2H3-D105R.H | 247 |
| Bis-40E9-L2H3-Y101F.H | 207 |
| Bis-40E9-L2H3-Y54G.H | 137 |
| Bis-40E9-L2H4 | 328 |
| Bis-40E9-L3H1 | 142 |
| Bis-40E9-L2H3-D99E.H | 128 |
| Bis-40E9-L2H1 | 113 |
| Bis-40E9-L2H3-G106A.H | 114 |
| ATG-Bis-40E9 | 490 |
| RG6234 | 836 |
| JNJ-64407564 | 1034 |

3.3 Humanized 2+1 Bispecific Antibody Characterization: Binding affinity to different multiple myeloma cell lines

The binding affinities of the generated humanized Fab-scFv-Fab bispecific antibodies to different multiple myeloma cell lines were evaluated by FACS analysis. The protocol for FACS analysis is identical to that described in Example 3.2, except that different cells were used in this example, i.e., multiple myeloma cell lines (MM.1S, MOLP-9, L363 and RPMI 8226) were used in this example.

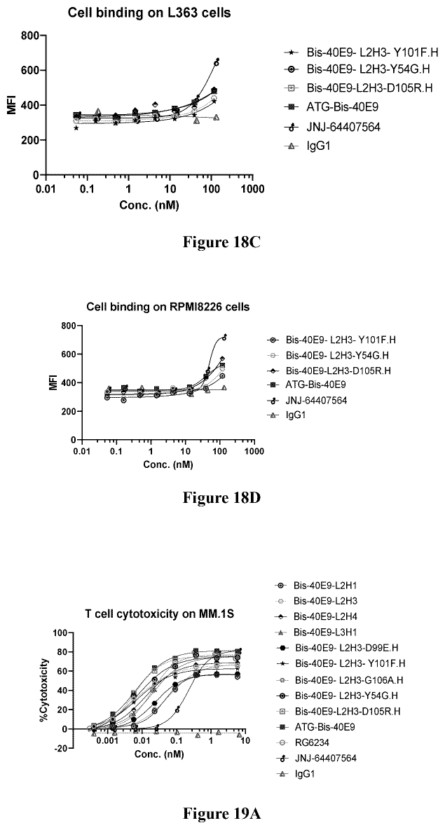

As shown in Table 8 and FIGS. 18A-D, the generated 3 humanized Fab-scFv-Fab bispecific antibodies showed comparable EC50 values and Top MFI on MM.1S, MOLP-8 cells, L363 and RPMI8226.

TABLE 8
EC₅₀ and Top MFI of binding affinity of humanized Fab-scFv-Fab bispecific antibody candidates

| | MM.1S | | MOLP-8 | | L363 | | RPMI8226 | |
|---|---|---|---|---|---|---|---|---|
| | EC₅₀ | Top | EC₅₀ | Top | EC₅₀ | Top | EC₅₀ | Top |
| Antibody | (nM) | (MFI) | (nM) | (MFI) | (nM) | (MFI) | (nM) | (MFI) |
| Bis-40E9-L2H3-D105R.H | 6.159 | 1066.00 | 0.2104 | 1093 | unstable | unstable | unstable | unstable |
| Bis-40E9-L2H3-Y54G.H | 8.932 | 1326.00 | 0.3976 | 951.8 | unstable | unstable | unstable | unstable |
| Bis-40E9-L2H3-Y101F.H | 10.14 | 1147.00 | 0.4664 | 875.6 | unstable | unstable | unstable | unstable |
| ATG-Bis-40E9 | 11.32 | 1198.00 | 0.8167 | 958.1 | unstable | unstable | unstable | unstable |
| JNJ-64407584 | 101.3 | 801.80 | 28.61 | 620.6 | unstable | unstable | unstable | unstable |

### 3.4 Humanized 2+1 Bispecific Antibody Characterization: T-Cell Dependent Cytotoxicity

The cytotoxicity of humanized Fab-scFv-Fab bispecific antibodies on different myeloma cell lines (MM.1S, MOLP-8, RPMI8226) and GPRC5D negative expressed cells (Daudi) were tested by FACS analysis. The protocol for FACS analysis is identical to that described in Example 1.3, except that different test antibodies were used.

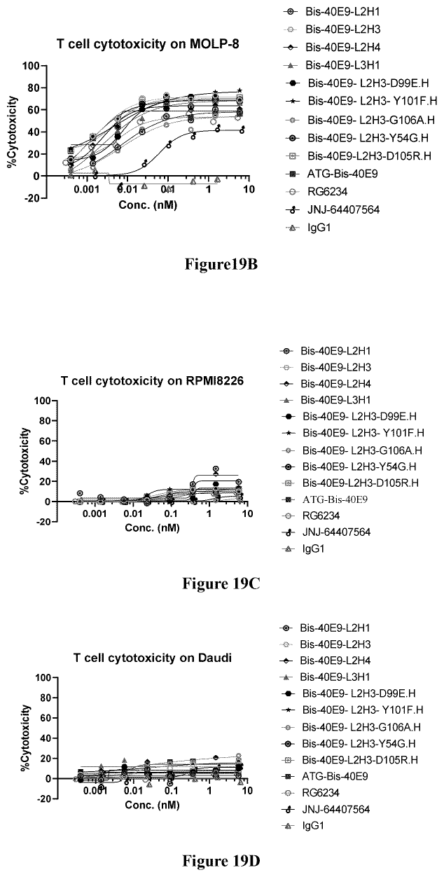

As shown in Table 9 and FIGS. 19A-D, the generated humanized Fab-scFv-Fab bispecific antibodies showed potent T cell dependent cytotoxicity on MM.1S, MOLP-8, and RPMI8226 cells, and had lower IC₅₀ values compared with the benchmark antibody JNJ-64407564. Among them, the top 4 candidate antibodies (Bis-40E9-L2H3, Bis-40E9-L2H3-Y54G.H, Bis-40E9-L2H3-D105R.H, Bis-40E9-L2H3-Y101F.H) had better T cell cytotoxicity effects compared with two benchmark antibodies JNJ-64407564 and RG6234.

TABLE 9
Top target cells killing percentage and IC₅₀ of humanized Fab-scFv-Fab
bispecific antibodies induced by T-Cell Dependent Cytotoxicity

| | MM.1S | | MOLP-8 | | RPMI8226 | | Daudi | |
|---|---|---|---|---|---|---|---|---|
| | IC₅₀ | Top | IC₅₀ | Top | IC₅₀ | Top | IC₅₀ | Top |
| Antibody | (nM) | (%) | (nM) | (%) | (nM) | (%) | (nM) | (%) |
| Bis-40E9-L2H3 | 0.00571 | 66.75 | 0.00166 | 71.92 | 0.0600 | 11.52 | 0.0118 | 11.86 |
| Bis-40E9-L2H3- | 0.00579 | 75.69 | 0.00463 | 70.14 | 0.0366 | 12.41 | 0.0030 | 7.903 |
| D105R.H | | | | | | | | |
| Bis-40E9-L2H3- | 0.00723 | 62.8 | 0.00499 | 77.09 | 0.0226 | 12.15 | Unstable | 3.498 |
| Y101F.H | | | | | | | | |
| Bis-40E9-L2H3- | 0.00726 | 75.48 | 0.00117 | 68.74 | 0.0453 | 10.37 | 0.0052 | 5.38 |
| Y54G.H | | | | | | | | |
| Bis-40E9-L2H4 | 0.01252 | 68.95 | 0.00239 | 63.89 | 0.3806 | 20.45 | 0.0264 | 16.19 |
| Bis-40E9-L3H1 | 0.02029 | 74.92 | 0.00244 | 68.33 | 0.2918 | 13.63 | Unstable | 11.93 |
| Bis-40E9-L2H3- | 0.02445 | 57.23 | 0.00776 | 68.73 | 0.4060 | 13.45 | Unstable | 8.25 |
| D99E.H | | | | | | | | |
| Bis-40E9-L2H1 | 0.04365 | 56.3 | 0.00690 | 58.28 | 0.3562 | 25.95 | Unstable | Unstable |
| Bis-40E9-L2H3- | 0.05704 | 67.43 | 0.00680 | 54.26 | 4.0370 | 5.692 | Unstable | 27.13 |
| G106A.H | | | | | | | | |
| ATG-Bis-40E9 | 0.00641 | 81.5 | 0.00607 | 58.98 | 0.0351 | 9.004 | 0.0089 | 14.18 |
| RG6234 | 0.01903 | 77.15 | 0.00599 | 53.4 | 0.0899 | 8.1 | 0.0860 | 4.867 |
| JNJ-64407564 | 0.22770 | 81.55 | 0.06131 | 41.52 | 1.2390 | 5.231 | 0.2965 | 11.23 |

3.5 Humanized 2+1 Bispecific Antibody Characterization: T Cell Activation with Jurkat NEAT Reporter Cell Line

The T cell activation effects of these generated humanized Fab-scFv-Fab bispecific antibodies were tested by Jurkat-NFAT-Luciferase assay, wherein different multiple myeloma cell lines (MM.1S, MOLP-8, L363, RPMI8226) and GPRC5D negative expressed Daudi were used as target cell and Jurkat was used as effector cell. The protocol is similar to that described in Example 1.4, except that different test antibodies were used.

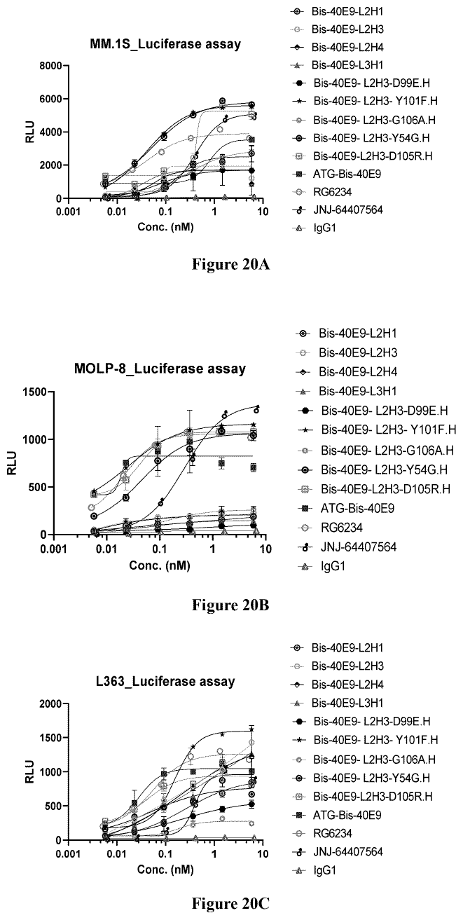

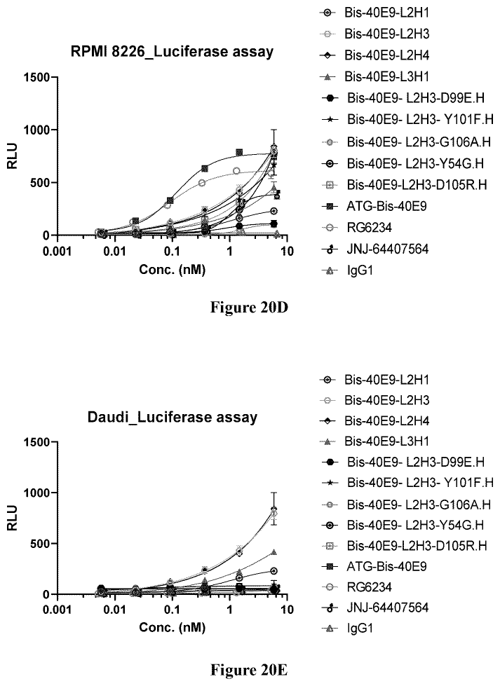

As shown in Table 10 and FIGS. 20A-E, the generated humanized Fab-scFv-Fab bispecific antibodies showed potent T cell activation on different multiple myeloma cell lines.

TABLE 10
Top RLU and EC₅₀ of humanized Fab-scFv-Fab bispecific antibodies induced by luciferase assay

| | MM.1S | | MOLP-8 | | L363 | | RPMI8226 | | Daudi | |
|---|---|---|---|---|---|---|---|---|---|---|
| | EC₅₀ | Top | EC₅₀ | Top | EC₅₀ | Top | EC₅₀ | Top | EC₅₀ | Top |
| Antibody | (nM) | (RLU) | (nM) | (RLU) | (nM) | (RLU) | (nM) | (RLU) | (nM) | (RLU) |
| Bis-40E9- | 0.08982 | 1941 | 2.096E−09 | 220.7 | Unsta- | Unsta- | Unsta- | Unsta- | Unsta- | 796.5 |
| L2H3 | | | | | ble | ble | ble | ble | ble | |
| Bis-40E9- | 0.4234 | 5238 | 0.04233 | 1079 | 0.03971 | 942 | Unsta- | Unsta- | Unsta- | 49.5 |
| L2H3- | | | | | | | ble | ble | ble | |
| D105R.H | | | | | | | | | | |
| Bis-40E9- | 0.03983 | 5629 | 1.005E−09 | 1473 | 0.1552 | 1598 | Unsta- | Unsta- | Unsta- | 94.5 |
| L2H3- | | | | | | | ble | ble | ble | |
| Y101F.H | | | | | | | | | | |
| Bis-40E9- | 0.05057 | 5836 | 0.04546 | 1065 | 0.05254 | 775.9 | Unsta- | Unsta- | Unsta- | 46 |
| L2H3- | | | | | | | ble | ble | ble | |
| Y54G.H | | | | | | | | | | |
| Bis-40E9- | 0.05916 | 1700 | 0.0005129 | 213.2 | 0.2061 | 1430 | Unsta- | Unsta- | Unsta- | 842 |
| L2H4 | | | | | | | ble | ble | ble | |
| Bis-40E9- | 0.03319 | 1712 | 0.07201 | 150.4 | 1.707 | 2755 | Unsta- | Unsta- | Unsta- | 417 |
| L3H1 | | | | | | | ble | ble | ble | |
| Bis-40E9- | 0.07192 | 1715 | 0.9059 | 117.5 | 0.1903 | 584.2 | 0.8785 | 113.4 | Unsta- | 50.5 |
| L2H3- | | | | | | | | | ble | |
| D99E.H | | | | | | | | | | |
| Bis-40E9- | 0.1803 | 2509 | 0.006537 | 1187 | 0.2578 | 898.2 | 1.051 | 261.2 | Unsta- | 229 |
| L2H1 | | | | | | | | | ble | |
| Bis-40E9- | 0.2677 | 2909 | 0.1164 | 262.4 | 0.1874 | 277 | 1.579 | 101.2 | Unsta- | 60.5 |
| L2H3- | | | | | | | | | ble | |
| G106A.H | | | | | | | | | | |
| ATG- | 0.7866 | 3551 | 0.01859 | 825.9 | 0.02805 | 1049 | 0.1171 | 776.1 | Unsta- | 38 |
| Bis-40E9 | | | | | | | | | ble | |
| RG6234 | 0.03972 | 3912 | 0.02101 | 1062 | 0.05319 | 1264 | 0.09291 | 615.6 | Unsta- | 37 |
| | | | | | | | | | ble | |
| JNJ- | 0.4002 | 5107 | 0.292 | 1377 | 0.3988 | 959.6 | 1.02 | 389 | Unsta- | 52.5 |
| 64407564 | | | | | | | | | ble | |

### 3.6 Humanized 2+1 Bispecific Antibody Characterization: Cytokines Release in PBMC

The biosafety of the top 4 candidate antibodies (namely, Bis-40E9-L2H3, Bis-40E9-L2H3-Y54G.H, Bis-40E9-L2H3-D105R.H, Bis-40E9-L2H3-Y101F.H) was evaluated by cytokines release of PBMC. The protocol is similar to that described in Example 1.5, except that different test antibodies were used.

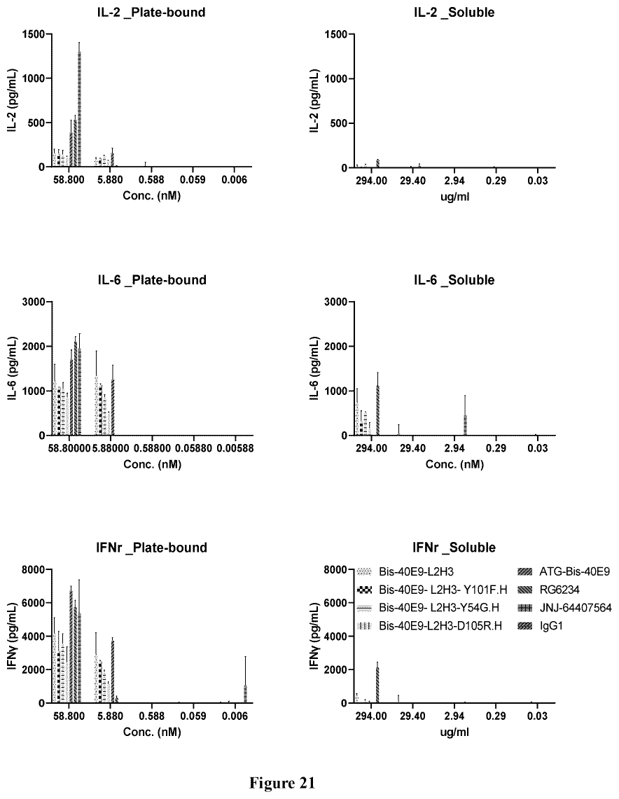

As shown in FIG. 21, these 4 candidate antibodies showed less IL-2, IFNγ and IL-6 release both in plate-bound or soluble antibody treatment at top concentration compared to benchmark antibodies JNJ-64407564 and RG6234. Therefore, these antibodies showed potent biosafety in PBMC activation compared with benchmark antibodies JNJ-64407564 and RG6234.

3.7 Humanized 2+1 Bispecific Antibody Characterization: Cytokines Release in T-Cell Dependent Cytotoxicity assay

The cytotoxicity of the generated humanized Fab-scFv-Fab bispecific antibody candidates on different multiple myeloma cell lines (MM.1S, MOLP-8, RPMI8226 and Daudi) cells was tested by FACS analysis. The protocol for FACS analysis is described in Example 1.2, except that different test antibodies were used.

The supernatants were analyzed with IL-2 (BD Biosciences, cat #555142), IFNγ (BD Biosciences, cat #353377) preformed according to the manufacturer's instructions.

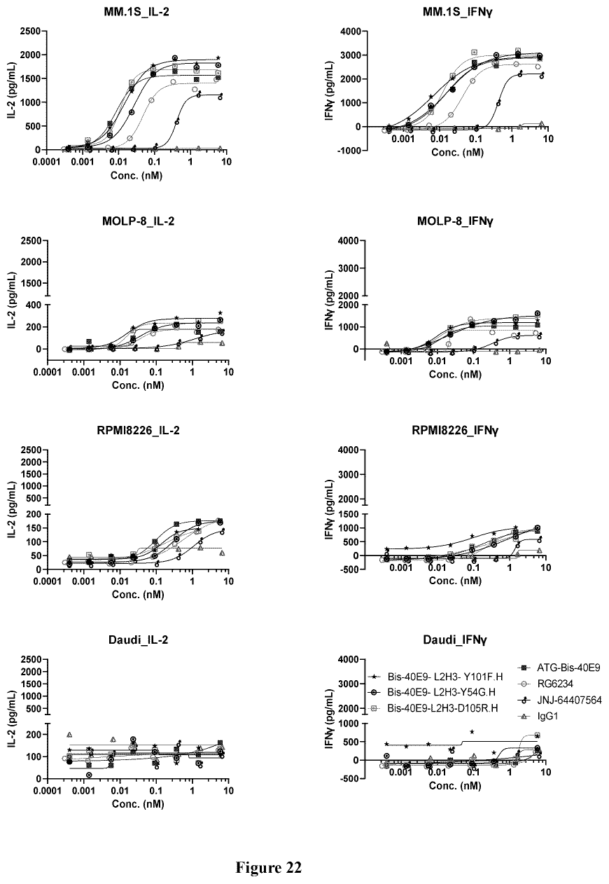

As shown in Table 20A-B and FIG. 22, the generated antibody candidates showed lower IC₅₀ of IL-2 and IFNγ release which was consistent with the T-cell cytotoxicity results.

TABLE 20A
IC₅₀ and Top IL-2 release of humanized Fab-scFv-Fab bispecific
antibody candidates in T-Cell Dependent Cytotoxicity

| | IL-2 secretion | | | | | | | |
|---|---|---|---|---|---|---|---|---|
| | MM.1S | | MOLP-8 | | RPMI8226 | | Daudi | |
| | IC₅₀ | Top | IC₅₀ | Top | IC₅₀ | Top | IC₅₀ | Top |
| | (nM) | (pg/ml) | (nM) | (pg/ml) | (nM) | (pg/ml) | (nM) | (pg/ml) |
| Bis-40E9-L2H3- | 0.0116 | 1689 | 0.016 | 234.9 | 0.1761 | 154 | Unstable | 113.5 |
| D105R.H | | | | | | | | |
| Bis-40E9-L2H3- | 0.0146 | 1897 | 0.016 | 276.9 | 0.2975 | 175.8 | Unstable | 129.6 |
| Y101F.H | | | | | | | | |
| Bis-40E9-L2H3- | 0.0272 | 1832 | 0.036 | 237.2 | 0.2975 | 175.8 | Unstable | 117.5 |
| Y54G.H | | | | | | | | |
| ATG-Bis-40E9 | 0.0085 | 1567 | 0.023 | 178.6 | 0.099 | 176.8 | Unstable | 162 |
| RG6234 | 0.0476 | 1401 | 0.035 | 179.50 | 0.5779 | 194.60 | Unstable | 157.20 |
| JNJ-64407564 | 0.4103 | 1156 | 1.158 | 175.9 | 1.078 | 146 | Unstable | 109.5 |

TABLE 20B
IC₅₀ and Top IFNγ release of humanized Fab-scFv-Fab
bispecific antibody candidates in T-Cell Dependent Cytotoxicity

| | IFNr secretion | | | | | | | |
|---|---|---|---|---|---|---|---|---|
| | MM.1S | | MOLP-8 | | RPMI8226 | | Daudi | |
| | IC₅₀ | Top | IC₅₀ | Top | IC₅₀ | Top | IC₅₀ | Top |
| | (nM) | (pg/ml) | (nM) | (pg/ml) | (nM) | (pg/ml) | (nM) | (pg/ml) |
| Bis-40E9-L2H3- | 0.01254 | 2998 | 0.01311 | 1398 | 0.6894 | 1440 | 1.79 | 677.5 |
| D105R.H | | | | | | | | |
| Bis-40E9-L2H3- | 0.006879 | 2948 | 0.009066 | 1196 | 0.09986 | 1061 | Unstable | 509.7 |
| Y101F.H | | | | | | | | |
| Bis-40E9-L2H3- | 0.01605 | 3104 | 0.02268 | 1528 | 0.984 | 1389 | 0.5004 | 334.7 |
| Y54G.H | | | | | | | | |
| ATG-Bis-40E9 | 0.01421 | 2887 | 0.01296 | 1054 | 0.1648 | 950.4 | 190.2 | 260 |
| RG6234 | 0.04291 | 2617 | 0.02169 | 852.90 | 0.2867 | 966.30 | 263.6 | 223.00 |
| JNJ-64407564 | 0.4216 | 2206 | 0.3244 | 622.2 | 1.411 | 585.6 | Unstable | 135 |

3.8 Humanized 2+1 Bispecific Antibody Characterization: In vivo Efficacy

To assess the activity of the generated humanized Fab-scFv-Fab bispecific antibodies in vivo, human PBMCs humanized MM.1S model was used, which was generated by using a similar protocol as described in Example 1.6, except that different test antibodies were used. ATG-Bis-40E9 was used as the parental antibody control of these humanized bispecific antibodies and JNJ-64407564 was used as the benchmark antibody. The tumor growth inhibition mean was calculated by using the same equation as described in Example 1.6.

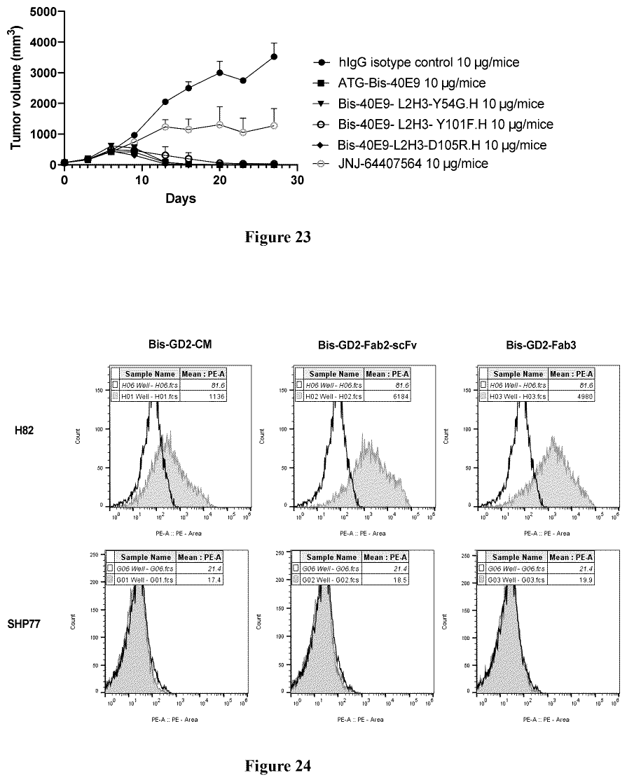

As shown in Table 11 and FIG. 23, all of the generated humanized Fab-scFv-Fab bispecific antibodies exhibited more potent in vivo anti-tumor efficacy than benchmark antibody JNJ-64407564, suggesting that they had better therapeutic effect.

TABLE 11
Tumor growth inhibition mean of humanized Fab-scFv-Fab bispecific antibodies

| hIgG isotype | Clone No. | | | | | | | | |
|---|---|---|---|---|---|---|---|---|---|
| control | D0 | D3 | D6 | D9 | D13 | D16 | D20 | D23 | D27 |

| | | | | | | | | |
|---|---|---|---|---|---|---|---|---|
| ATG- | −0.21% | 4.07% | 61.60% | 99.75% | 102.00% | 102.46% | 102.69% | 102.08% |
| Bis-40E9 | | | | | | | | |
| Bis-40E9- | −14.47% | −14.17% | 56.30% | 87.89% | 94.86% | 100.55% | 101.22% | 101.10% |
| L2H3- | | | | | | | | |
| Y101F.H | | | | | | | | |
| Bis-40E9- | −38.93% | −46.49% | 46.73% | 99.20% | 102.33% | 102.46% | 102.69% | 102.08% |
| L2H3- | | | | | | | | |
| Y54G.H | | | | | | | | |
| Bis-40E9- | −7.43% | −2.25% | 73.85% | 102.12% | 102.74% | 102.46% | 102.68% | 102.08% |
| L2H3- | | | | | | | | |
| D105R.H | | | | | | | | |
| JNJ- | −24.28% | 4.65% | 25.60% | 41.44% | 55.47% | 57.82% | 63.25% | 65.10% |
| 64407564 | | | | | | | | |

### Example 4. GD2×CD3 Bispecific Antibody Generation and Characterization

### 4.1 GD2×CD3 Bispecific Antibody Generation

Bispecific antibody targeting GD2 and CD3 were generated to evaluate the antibody characterization, including binding affinity, T cell dependent cytotoxicity and T cell activation with Jurkat NFAT luciferase reporter cells. Three formats (namely, Crossmab, Fab-scFv-Fab, and Fab3) were used as the formats of the generated bispecific antibodies. The diagrams for the three formats are shown in FIG. 30.

The Crossmab format is described in WO2009/080253A1. In general, the generated Crossmab GD2×CD3 bispecific antibody is an IgG-like bispecific antibody, comprises an anti-GD2 Fab domain, an anti-CD3 Fab domain, and constant domains, wherein the anti-GD2 Fab domain comprises a GD2 binding moiety comprising a VH region and a VL region of humanized 3F8 as described in US20130216528A1, the anti-CD3 Fab domain comprises a CD3 binding moiety comprising a VH region and a VL region of 40-C12-C10-E9, and the CH1 and CL regions of the anti-CD3 Fab domain were replaced by each other; the constant regions are human IgG1 constant region, and LALA mutation (L234A, L235A, Eu numbering) was introduced to the IgG1 Fc region to abolish the Fc receptor binding. In addition, knob-in-hole mutation (S354C, T366W on CH3 of a first heavy chain, Y349C, T366S, L368A, Y407V on CH3 of a second heavy chain) was introduced to avoid heavy chain mismatch.

The Fab-scFv-Fab format is described in WO2016086189A2. In general, the generated Fab-scFv-Fab GD2×CD3 bispecific antibody comprises two anti-GD2 Fab domains and one anti-CD3 scFv domain, wherein each of the anti-GD2 Fab domains comprises a GD2 binding moiety comprising a VH region and a VL region of humanized 3F8 as described in US20130216528A1, and the anti-CD3 scFv domain comprises the VH and VL regions from clone 40-C12-C10-E9, wherein the VH and VL regions of the anti-CD3 scFv domain were linked by a (GGGGS)₄ linker, and the VL region of the anti-CD3 scFv domain was linked to the N-terminal of the CH2 domain of the chimeric antibody by a (GGGGS)₂ linker, the VH region of the anti-CD3 scFv domain was linked to CH1 domain of one of the anti-GD2 Fab domains by a (GGGGS)₂ linker. LALA mutations (L234A, L235A, according to Eu numbering) was introduced to each bispecific antibody to abolish the Fc receptor binding capability.

The Fab3 format is described in WO2019154890A1. In general, the generated Fab3 GD2×CD3 bispecific antibody comprises two anti-GD2 Fab domains and one anti-CD3 Fab domain, wherein each of the anti-GD2 Fab domains comprises a GD2 binding moiety comprising a VH region and a VL region of humanized 3F8 as described in US20130216528A1, and the anti-CD3 Fab domain comprises a CD3 binding moiety comprising a VH region and a VL region of 40-C12-C10-E9.

The generated Crossmab, Fab-scFv-Fab and Fab3 GD2×CD3 bispecific antibodies had the same VH and VL sequences of anti-GD2 and anti-CD3 domains. In particular, the VH and VL regions of the anti-GD2 domain comprise an amino acid sequence as set forth in SEQ ID NO: 19 and SEQ ID NO: 20, respectively; the VH and VL regions of the anti-CD3 domain comprise an amino acid sequence as set forth in SEQ ID NO: 3 and SEQ ID NO: 4, respectively. The generated GD2×CD3 bispecific antibodies are also named as “Bis-GD2-CM”, “Bis-GD2-Fab2-scFv”, and “Bis-GD2-Fab3”, respectively, wherein the prefix “Bis-” indicates “bispecific”, and the suffix “-CM” indicates “Crossmab” format, the suffix “Fab2-scFv” indicates “Fab-scFv-Fab” format, and the suffix “Fab3” indicates the Fab3 format. The amino acid sequences of SEQ ID NOs: 19 and 20 are shown in Table 19 below, and CDR sequences identified by the convention of Kabat are underlined in each sequence.

TABLE 19
Amino Acid Sequences of VH and VL regions
of Humanized 3F8

| Sequence | | SEQ ID | |
|---|---|---|---|
| Description | | NO | Amino Acid Sequence |
| Humanized | VH | 19 | QVQLVESGPGVVQPGRSLRIS |
| | | | CAVSGFSVTNYGVHWVRQPPG |
| | | | KGLEWLGVIWAGGITNYNSAF |
| | | | MSRLTISKDNSKNTVYLQMNS |
| | | | LRAEDTAMYYCASRGGHYGYA |
| | | | LDYWGQGTLVTVSS |
| 3F8 | VL | 20 | EIVMTQTPATLSVSAGERVTI |
| | | | TCKASQSVSNDVTWYQQKPGQ |
| | | | APRLLIYSASNRYSGVPARFS |
| | | | GSGYGTEFTFTISSVQSEDFA |
| | | | VYFCQQDYSSFGQGTKLEIK |

### 4.2 GD2×CD3 Bispecific Antibodies Characterization: Binding Affinity to Non-Small-Cell Lung Cancer (NSCLC) Cell Line and T Cells

To evaluate the binding affinity of the generated bispecific antibodies to NSCLC cells and T cells, the NSCLC cells (H82 and SHP77) and T cells (CD3⁺ T cells isolated from normal donors) were stained by the test antibodies and the binding affinity was determined by FACS analysis. The protocol of FACS analysis is described as follows:
(a) NSCLC cells and T cells were washed by Staining Buffer (PBS+1% BSA) in round bottom 96-well plate.
(b) Antibody dilutions and cells were added to each well and mixed with the cells, incubated at 4 degrees for 1 hour.
(c) Cells were spined at 300 g for 3 minutes, then the supernatants were discarded.
(d) Cells were washed with Staining Buffer.
(e) Step (d) was repeated, and the cells were incubated with secondary antibody in Staining Buffer for 0.5 hours at 4 degrees.
(f) After incubation, step (d)-(e) were repeated.
(g) Cells were resuspended in Staining Buffer and fluorescence signals were recorded by flow cytometry.

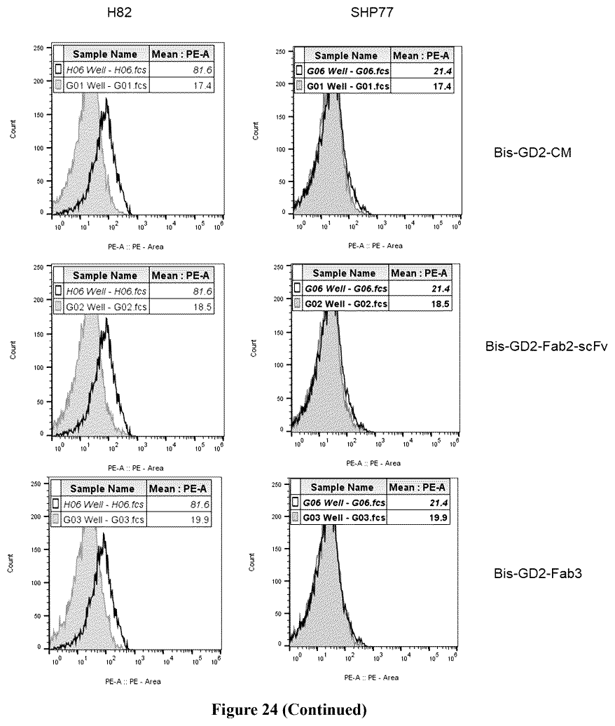

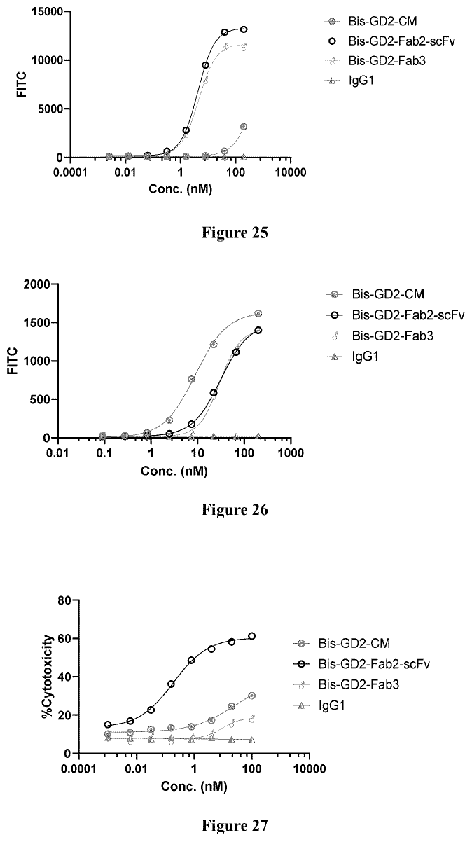

As shown in FIG. 24, GD2 was positively expressed on H82 cells and was negative on SHP77 cells. As shown in FIG. 25, Bis-GD2-Fab2-scFv bound to the H82 cells with a lower EC50 and higher affinity than Bis-GD2-CM and identical affinity compared to Bis-GD2-Fab3. As shown in FIG. 26, Bis-GD2-Fab2-scFv also showed comparable binding affinity to T cells compared with Bis-GD2-Fab3.

### 4.3 GD2×CD3 Bispecific Antibodies Characterization: T-Cell Dependent Cytotoxicity

The cytotoxicity of CD3×GD2 bispecific antibodies on NSCLC cell line (H82 cell line) was tested by FACS analysis. The protocol for FACS analysis is similar to that described in Example 1.3, except that the different cell line and different test antibodies were used.

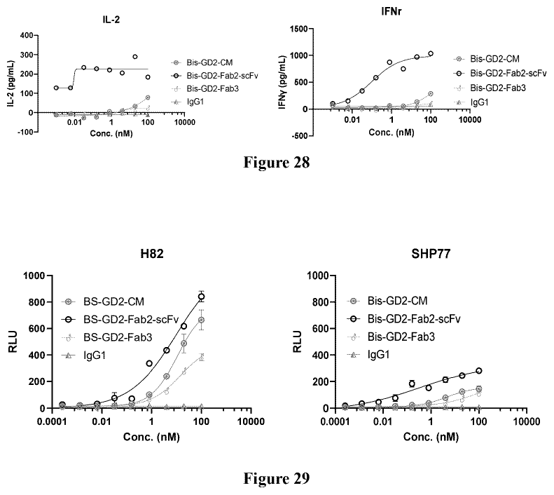

As shown in Table 12 and FIG. 27, Bis-GD2-Fab2-scFv showed potent T cell dependent cytotoxicity on H82 cells, and had lower IC₅₀ and higher top cytotoxicity precent compared with Bis-GD2-CM and Bis-GD2-Fab3. In term of cytokines release, as shown in FIG. 28, Bis-GD2-Fab2-scFv showed higher IL-2 and IFNγ release compared with Bis-GD2-CM and Bis-GD2-Fab3. These data suggested that Fab-scFv-Fab format of GD2×CD3 bispecific antibody had higher T cell cytotoxicity than Crossmab format and Fab3 format.

TABLE 12
Top target cells killing percentage and IC50 of CD3 x GD2 bispecific
antibodies induced by T-Cell Dependent Cytotoxicity

| | H82 Cells | |
|---|---|---|
| | IC₅₀ | TOP |
| Antibody | (nM) | (%) |
| Bis-GD2-Fab2-scFv | 0.19 | 60.43 |
| Bis-GD2-Fab3 | 9.74 | 18.69 |
| Bis-GD2-CM | 27.07 | 39.33 |
| IgG1 | Unstable | Unstable |

4.4 GD2×CD3 Bispecific Antibodies Characterization: T Cell Activation with Jurkat NEAT Reporter Cell Line

The T cell activation effects of the generated GD2×CD3 bispecific antibodies were tested by Jurkat-NFAT-Luciferase assay, wherein NSCL cell line (H82 cell line) and GD2 negative-expressed SHP77 cells were used as target cell and Jurkat was used as effector cell. The protocol is similar to that described in Example 1.4, except that different test antibodies were used.

As shown in Table 13 and FIG. 29, the generated Bis-GD2-Fab2-scFv showed potent T cell activation on and higher top RLU values compared with Bis-GD2-CM and Bis-GD2-Fab3. In summary, Fab-scFv-Fab format of GD2×CD3 bispecific antibody had higher T cell activation than Crossmab format and Fab3 format.

TABLE 13
Top RLU and EC₅₀ of GD2 x CD3 bispecific antibodies induced
by luciferase assay

| | H82 | | SHP77 | |
|---|---|---|---|---|
| | EC₅₀ | Top | EC₅₀ | Top |
| | (nM) | (RLU) | (nM) | (RLU) |
| Bis-GD2-Fab2-scFv | 9.85 | 1109 | 0.33 | 333.8 |
| Bis-GD2-Fab3 | 14.22 | 476.1 | 252.3 | 283.5 |
| Bis-GD2-CM | 10.77 | 768.6 | 4.7 | 158.4 |
| IgG1 | Unstable | 14.48 | Unstable | 7.83 |

### Example 5. Humanized 2+1 Bispecific Antibody Generation and Characterization

### 5.1 Humanized 2+1 Bispecific Antibody Generation

Bispecific antibodies targeting GPRC5D and CD3 were generated and characterized, wherein the characterizations include binding affinity to Jurkat and MM.1S cells, T cell dependent cytotoxicity and T cell activation with Jurkat NFAT luciferase reporter cells. Fab-scFv-Fab format was used in generating the bispecific antibodies. In particular, each of the humanized Fab-scFv-Fab bispecific antibodies generated herein comprises two humanized anti-GPRC5D Fab domains and one anti-CD3 scFv domain, wherein each of the humanized anti-GPRC5D Fab domains comprises a GPRC5D binding moiety comprising a humanized VH region and a humanized VL region from the clone 108C4C12, and the anti-CD3 scFv domain comprises a parental VH region and a parental VL region of clone 147E11E2 (named as Bis-108+ch147) or a humanized and affinity matured VH region and a humanized and affinity matured VL region from the clone 147E11E2, wherein the humanized VH region of each of the anti-GPRC5D Fab domains comprises an amino acid sequence as set forth in SEQ ID NO: 15, and the humanized VL region of each of the anti-GPRC5D Fab domains comprises an amino acid sequence as set forth in SEQ ID NO: 18. The resulting humanized 2+1 bispecific antibodies are referred to herein as Bis-108+ch147, Bis-108+147-S31R.H, Bis-108+147-S54R.H, Bis-108+147-S102Q.H, Bis-108+147-R100Q.H, Bis-108+147-S32T.L, Bis-108+147-S32R.L, Bis-108+147-K36R.L, Bis-108+147-N31Y.L, Bis-108+147-R35K.L, Bis-108+147-R33A.L, Bis-108+147-R35S.L, Bis-108+147-N37K.L. The SEQ ID NOs and amino acid sequences of VL and VH regions of the anti-CD3 scFv domain in each of the generated humanized 2+1 bispecific antibodies are shown in Table 30 and Table 31 below, and CDR sequences identified by the convention of Kabat are underlined in each sequence listed in Table 31.

TABLE 30
VH and VL Regions of the anti-CD3 scFv Domain of the Generated
Exemplary Bispecific Antibodies

| | | VH SEQ | VL | VL SEQ |
|---|---|---|---|---|
| BsAb name | VH Region | ID NO | Region | ID NO |
| Bis-108+ch147 | 147E11E2 VH | 205 | 147E11E2 | 206 |
| | | | VL | |
| Bis-108+147- | 147-VH3b- | 208 | 147-VL1 | 212 |
| S31R.H | S31R.H | | | |
| Bis-108+147- | 147-VH3b- | 209 | 147-VL1 | 212 |
| S54R.H | S54R.H | | | |
| Bis-108+147- | 147-VH3b- | 210 | 147-VL1 | 212 |
| S102Q.H | S102Q.H | | | |
| Bis-108+147- | 147-VH3b- | 211 | 147-VL1 | 212 |
| R100Q.H | R100Q.H | | | |
| Bis-108+147- | 147-VH3b | 207 | 147-VL1- | 213 |
| S32T.L | | | S32T.L | |
| Bis-108+147- | 147-VH3b | 207 | 147-VL1- | 214 |
| S32R.L | | | S32R.L | |
| Bis-108+147- | 147-VH3b | 207 | 147-VL1- | 215 |
| K36R.L | | | K36R.L | |
| Bis-108+147- | 147-VH3b | 207 | 147-VL1- | 216 |
| N31Y.L | | | N31Y.L | |
| Bis-108+147- | 147-VH3b | 207 | 147-VL1- | 217 |
| R35K.L | | | R35K.L | |
| Bis-108+147- | 147-VH3b | 207 | 147-VL1- | 218 |
| R33A.L | | | R33A.L | |
| Bis-108+147- | 147-VH3b | 207 | 147-VL1- | 219 |
| R35S.L | | | R35S.L | |
| Bis-108+147- | 147-VH3b | 207 | 147-VL1- | 220 |
| N37K.L | | | N37K.L | |

TABLE 31
Amino Acid Sequences of VH and VL Regions of mouse or humanized
147E11E2 Used in the Humanized 2+1 Bispecific Antibodies

| | SEQ | Amino Acid Sequence |
|---|---|---|
| | ID | (the CDRs are bold and |
| Description | NO: | underlined) |
| 147E11E2 VH | 205 | QVQLQQPGTELVKPGTSVKLSCKASGYTFTSYW |
| (mouse) | | MHWVKQRPGQCLEWIGNINPSNGATNYNEKFKS |
| | | KATLTVDKSSSTAYMQLSSLTSEDSAVYYCARD |
| | | RSSQYYFDYWGQGTTLTVSS |
| 147E11E2 VL | 206 | DIVMSQSPSSLAVSAGEKVTMSCKSSQSLLNSRS |
| (mouse) | | RKNYLAWYQQKPGQSPKLLIYWASTRESGVPDRF |
| | | TGSGSGTDFTLTISSVQAEDLAVYYCTQSYTLRT |
| | | FGCGTKLEIK |
| 147-VH3b | 207 | EVQLVQSGAEVKKPGSSVKVSCKASGYTFTSYW |
| | | MHWVRQAPGQCLEWMGNINPSSGATNYNEKFKS |
| | | RVTLTVDKSTSTAYMELSSLRSEDTAVYYCARD |
| | | RSSQYYFDYWGQGTTVTVSS |
| 147-VH3b- | 208 | EVQLVQSGAEVKKPGSSVKVSCKASGYTFTRYW |
| S31R.H | | MHWVRQAPGQCLEWMGNINPSSGATNYNEKFKS |
| | | RVTLTVDKSTSTAYMELSSLRSEDTAVYYCARD |
| | | RSSQYYFDYWGQGTTVTVSS |
| 147-VH3b- | 209 | EVQLVQSGAEVKKPGSSVKVSCKASGYTFTSYW |
| S54R.H | | MHWVRQAPGQCLEWMGNINPRSGATNYNEKFKS |
| | | RVTLTVDKSTSTAYMELSSLRSEDTAVYYCARD |
| | | RSSQYYFDYWGQGTTVTVSS |
| 147-VH3b- | 210 | EVQLVQSGAEVKKPGSSVKVSCKASGYTFTSYW |
| S102Q.H | | MHWVRQAPGQCLEWMGNINPSSGATNYNEKFKS |
| | | RVTLTVDKSTSTAYMELSSLRSEDTAVYYCARD |
| | | RSQQYYFDYWGQGTTVTVSS |
| 147-VH3b- | 211 | EVQLVQSGAEVKKPGSSVKVSCKASGYTFTSYW |
| R100Q.H | | MHWVRQAPGQCLEWMGNINPSSGATNYNEKFKS |
| | | RVTLTVDKSTSTAYMELSSLRSEDTAVYYCARD |
| | | QSSQYYFDYWGQGTTVTVSS |
| 147-VL1 | 212 | DIVMTQSPDSLAVSLGERATINCKSSQSLLNSRS |
| (humanized) | | RKNYLAWYQQKPGQPPKLLIYWASTRESGVPDRF |
| | | SGSGSGTDFTLTISSLQAEDVAVYYCTQSYTLRT |
| | | FGCGTKVEIK |
| 147-VL1- | 213 | DIVMTQSPDSLAVSLGERATINCKSSQSLLNTRS |
| S32T.L | | RKNYLAWYQQKPGQPPKLLIYWASTRESGVPDRF |
| | | SGSGSGTDFTLTISSLQAEDVAVYYCTQSYTLRT |
| | | FGCGTKVEIK |
| 147-VL1- | 214 | DIVMTQSPDSLAVSLGERATINCKSSQSLLNRRS |
| S32R.L | | RKNYLAWYQQKPGQPPKLLIYWASTRESGVPDRF |
| | | SGSGSGTDFTLTISSLQAEDVAVYYCTQSYTLRT |
| | | FGCGTKVEIK |
| 147-VL1- | 215 | DIVMTQSPDSLAVSLGERATINCKSSQSLLNSRS |
| K36R.L | | RRNYLAWYQQKPGQPPKLLIYWASTRESGVPDRF |
| | | SGSGSGTDFTLTISSLQAEDVAVYYCTQSYTLRT |
| | | FGCGTKVEIK |
| 147-VL1- | 216 | DIVMTQSPDSLAVSLGERATINCKSSQSLLYSRS |
| N31Y.L | | RKNYLAWYQQKPGQPPKLLIYWASTRESGVPDRF |
| | | SGSGSGTDFTLTISSLQAEDVAVYYCTQSYTLRT |
| | | FGCGTKVEIK |
| 147-VL1- | 217 | DIVMTQSPDSLAVSLGERATINCKSSQSLLNSRS |
| R35K.L | | KKNYLAWYQQKPGQPPKLLIYWASTRESGVPDRF |
| | | SGSGSGTDFTLTISSLQAEDVAVYYCTQSYTLRT |
| | | FGCGTKVEIK |
| 147-VL1- | 218 | DIVMTQSPDSLAVSLGERATINCKSSQSLLNSAS |
| R33A.L | | RKNYLAWYQQKPGQPPKLLIYWASTRESGVPDRF |
| | | SGSGSGTDFTLTISSLQAEDVAVYYCTQSYTLRT |
| | | FGCGTKVEIK |
| 147-VL1- | 219 | DIVMTQSPDSLAVSLGERATINCKSSQSLLNSRS |
| R35S.L | | SKNYLAWYQQKPGQPPKLLIYWASTRESGVPDRF |
| | | SGSGSGTDFTLTISSLQAEDVAVYYCTQSYTLRT |
| | | FGCGTKVEIK |
| 147-VL1- | 220 | DIVMTQSPDSLAVSLGERATINCKSSQSLLNSRS |
| N37K.L | | RKKYLAWYQQKPGQPPKLLIYWASTRESGVPDRF |
| | | SGSGSGTDFTLTISSLQAEDVAVYYCTQSYTLRT |
| | | FGCGTKVEIK |

### 5.2 Humanized 2+1 Bispecific Antibody Characterization: Binding Affinity to Jurkat Cells

The binding affinities of the generated humanized Fab-scFv-Fab bispecific antibodies were determined by FACS analysis. The ATG-Bis-40E9 described in Example 1 was used as positive control and hIgG1 isotype was used as negative control. The protocol for FACS analysis is described in Example 3.2 above.

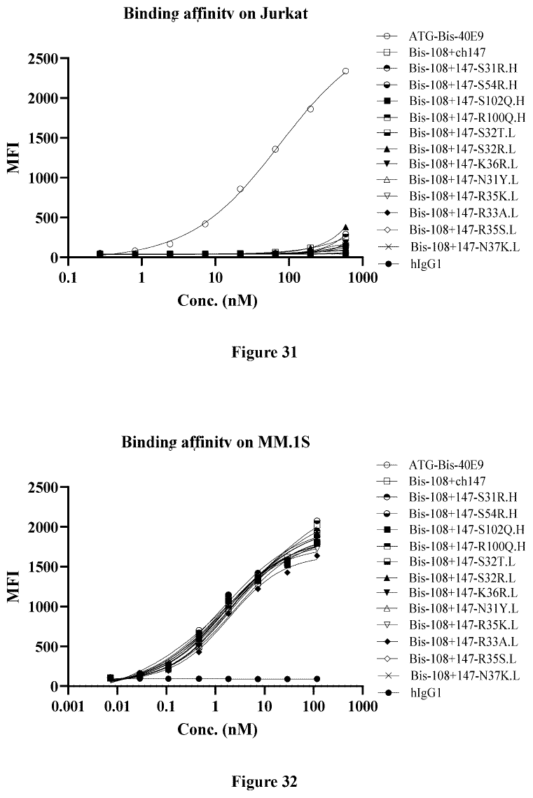

As shown in Table 32 and FIG. 31, the binding ability of the generated humanized Fab-scFv-Fab bispecific antibodies to Jurkat cells were lower than that of ATG-Bis-40E9.

TABLE 32
Top MFI and EC₅₀ of Jurkat cell binding affinity of
humanized 2+1 bispecific antibodies

| Ranking | Antibody | EC₅₀ (nM) | Top signal |
|---|---|---|---|
| 1 | ATG-Bis-40E9 | 74.68 | 2862 |
| 2 | Bis-108+147-S32R.L | — | 383 |
| 3 | Bis-108+147-S54R.H | — | 293 |
| 4 | Bis-108+ch147 | — | 248 |
| 5 | Bis-108+147-K36R.L | — | 187 |
| 6 | Bis-108+147-S32T.L | — | 158 |
| 7 | Bis-108+147-S31R.H | — | 156 |
| 8 | Bis-108+147-S102Q.H | — | 141 |
| 9 | Bis-108+147-R35K.L | — | 133 |
| 10 | Bis-108+147-N37K.L | — | 96.1 |
| 11 | Bis-108+147-R33A.L | — | 90.6 |
| 12 | Bis-108+147-R35S.L | — | 89.3 |
| 13 | Bis-108+147-N31Y.L | — | 62.8 |
| 14 | Bis-108+147-R100Q.H | — | 50.9 |

5.3 Humanized 2+1 Bispecific Antibody Characterization: Binding affinity to MM.1S Cells

The binding affinities of the generated humanized Fab-scFv-Fab bispecific antibodies were determined by FACS analysis. The ATG-Bis-40E9 was used as positive control and hIgG1 isotype was used as negative control. The protocol for FACS analysis is identical to that described in Example 3.2, except that MM.1S cells were used in this example.

As shown in Table 33 and FIG. 32, the binding ability of the generated humanized Fab-scFv-Fab bispecific antibodies to MM.1S cells were higher, lower or comparable than that of ATG-Bis-40E9.

TABLE 33
Top MFI and EC₅₀ of MM.1S cell binding affinity of
humanized 2+1 bispecific antibodies

| Ranking | Antibody | EC₅₀ (nM) | Top signal |
|---|---|---|---|
| 1 | Bis-108+147-S102Q.H | 1.29 | 1818 |
| 2 | Bis-108+147-S31R.H | 1.50 | 2026 |
| 3 | Bis-108+147-R35S.L | 1.53 | 1858 |
| 4 | Bis-108+147-K36R.L | 1.56 | 1845 |
| 5 | Bis-108+147-R35K.L | 1.58 | 1741 |
| 6 | Bis-108+147-S32R.L | 1.76 | 2052 |
| 7 | Bis-108+147-R33A.L | 1.87 | 1653 |
| 8 | Bis-108+147-R100Q.H | 1.87 | 1829 |
| 9 | Bis-108+147-N31Y.L | 1.93 | 1931 |
| 10 | Bis-108+147-S32T.L | 2.00 | 2041 |
| 11 | Bis-108+147-N37K.L | 2.15 | 1901 |
| 12 | ATG-Bis-40E9 | 2.43 | 1884 |
| 13 | Bis-108+ch147 | 2.61 | 2263 |
| 14 | Bis-108+147-S54R.H | 3.70 | 2624 |

### 5.4 Humanized 2+1 Bispecific Antibody Characterization: T-Cell Dependent Cytotoxicity

The cytotoxicity of the generated humanized Fab-scFv-Fab bispecific antibodies on GPRC5D positive expressed cell line (MM.1S) and GPRC5D negative expressed cell line (Daudi) were tested by FACS analysis. The protocol for FACS analysis is identical to that described in Example 1.3, except that different test antibodies were used.

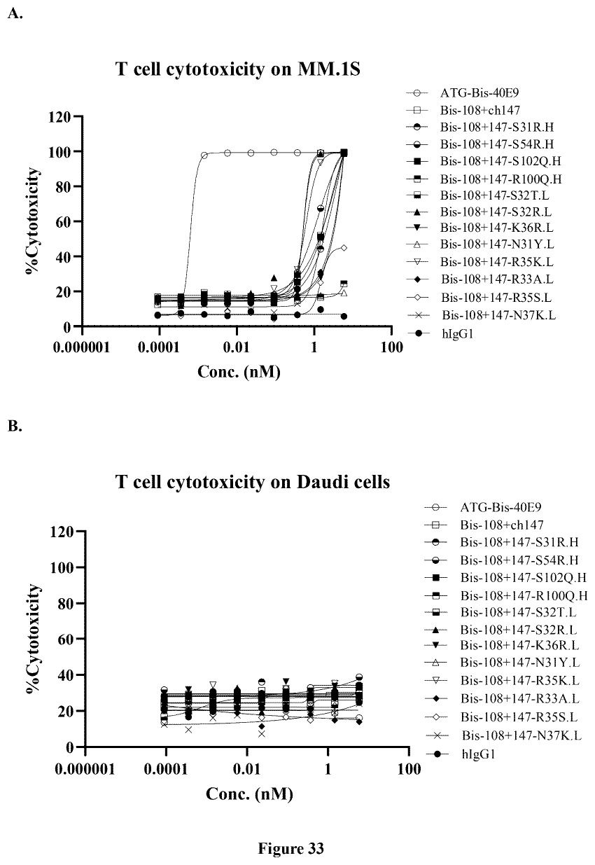

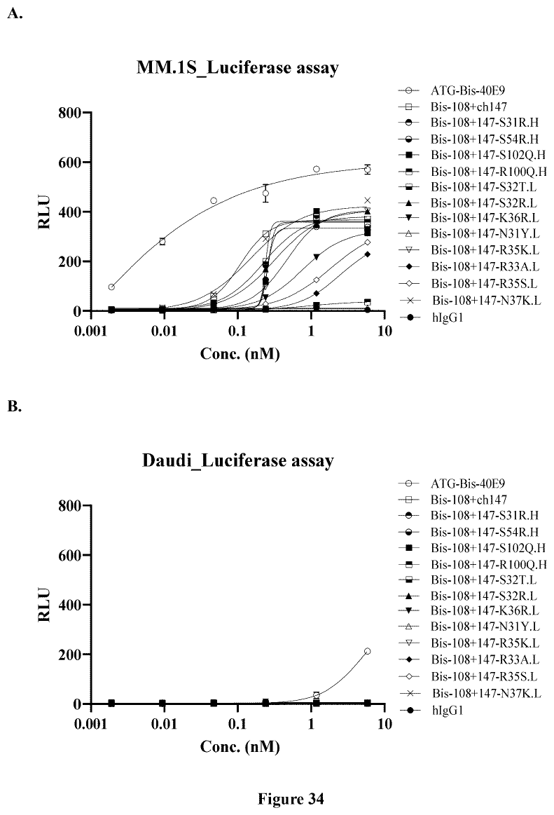

As shown in Table 34, FIGS. 33A and 33B, the humanized Fab-scFv-Fab bispecific antibodies generated herein showed T cell dependent cytotoxicity on MM.1S cells but not on Daudi cells.

TABLE 34
Top target cells killing percentage and IC₅₀ of humanized
Fab-scFv-Fab bispecific antibody induced by T-Cell
Dependent Cytotoxicity on MM.1S cells

| Ranking | Antibody | IC₅₀ (nM) | Top (%) |
|---|---|---|---|
| 1 | ATG-Bis-40E9 | 0.00063 | 99.22 |
| 2 | BIS-108+ch147 | 0.53 | 99.44 |
| 3 | BIS-108+147-S32R.L | 0.54 | 98.99 |
| 4 | BIS-108+147-R35K.L | 0.61 | 99.92 |
| 5 | BIS-108+147-S54R.H | 1.25 | 110.50 |
| 6 | BIS-108+147-R33A.L | 1.51 | 45.09 |
| 7 | BIS-108+147-R35S.L | 1.64 | 101.60 |
| 8 | BIS-108+147-S102Q.H | 2.78 | 131.40 |
| 9 | BIS-108+147-S31R.H | 4.43 | 158.80 |
| 10 | BIS-108+147-S32T.L | 4.76 | 166.30 |
| 11 | BIS-108+147-R100Q.H | 7.53 | 46.30 |
| 12 | BIS-108+147-N37K.L | 1489 | Unstable |
| 13 | BIS-108+147-K36R.L | 1939 | Unstable |
| 14 | BIS-108+147-N31Y.L | 1.01E+28 | 121.9 |

5.5 Humanized 2+1 Bispecific Antibody Characterization: T Cell Activation with Jurkat NFAT Reporter Cell Line

The T cell activation effects of these generated humanized Fab-scFv-Fab bispecific antibodies were tested by Jurkat-NFAT-Luciferase assay, wherein GPRC5D positive expressed cell line (MM.1S) and GPRC5D negative expressed cell line (Daudi) were used as target cell and Jurkat was used as effector cell. The ATG-Bis-40E9 was used as positive controls and hIgG1 isotype was used as negative control. The protocol is similar to that described in Example 1.4, except that different test antibodies were used.

As shown in Table 35, FIGS. 34A and 34B, the generated humanized Fab-scFv-Fab bispecific antibodies showed T cell activation effect in the presence of MM.1S cells which expressing GPRC5D.

TABLE 35
Top RLU and EC₅₀ of humanized Fab-scFv-Fab bispecific
antibodies induced by luciferase assay in the presence of
MM.1S cells

| Ranking | Antibody | EC₅₀ (nM) | Top RLU |
|---|---|---|---|
| 1 | ATG-Bis-40E9 | 0.011 | 563.60 |
| 2 | Bis-108+ch147 | 0.11 | 367.60 |
| 3 | Bis-108+147-N37K.L | 0.15 | 424.00 |
| 4 | Bis-108+147-S32T.L | 0.22 | 380.10 |
| 5 | Bis-108+147-S54R.H | 0.24 | 361.50 |
| 6 | Bis-108+147-S31R.H | 0.25 | 333.80 |
| 7 | Bis-108+147-S102Q.H | 0.26 | 358.30 |
| 8 | Bis-108+147-N31Y.L | 0.27 | 11.75 |
| 9 | Bis-108+147-S32R.L | 0.32 | 406.40 |
| 10 | Bis-108+147-R35K.L | 0.47 | 407.50 |
| 11 | Bis-108+147-K36R.L | 0.76 | 326.00 |
| 12 | Bis-108+147-R100Q.H | 0.98 | 39.23 |
| 13 | Bis-108+147-R35S.L | 1.87 | 343.70 |
| 14 | Bis-108+147-R33A.L | 2.48 | 287.30 |

### Example 6. Humanized 2+1 Bispecific Antibody Generation and Characterization

### 6.1 Humanized 2+1 Bispecific Antibody Generation

Bispecific antibodies targeting GPRC5D and CD3 were generated and characterized, wherein the characterizations include binding affinity to T cells, T cell dependent cytotoxicity, T cell activation with Jurkat NFAT luciferase reporter cells. Fab-scFv-Fab format was used in generating the bispecific antibodies. In particular, each of the humanized Fab-scFv-Fab bispecific antibodies generated herein comprises two humanized anti-GPRC5D Fab domains and one humanized and affinity matured anti-CD3 scFv domain, wherein each of the humanized anti-GPRC5D Fab domains comprises a GPRC5D binding moiety comprising a humanized VH region and a humanized VL region from the clone 108C4C12, and the humanized and affinity matured anti-CD3 scFv domain comprises a humanized and affinity matured VH region and a humanized and affinity matured VL region from the clone 40-C12-C10-E9, wherein the humanized VH region of each of the anti-GPRC5D Fab domains comprises an amino acid sequence as set forth in SEQ ID NO: 15, and the humanized VL region of each of the anti-GPRC5D Fab domains comprises an amino acid sequence as set forth in SEQ ID NO: 18, wherein the VH and VL regions of the humanized and affinity matured anti-CD3 scFv domain were linked by a (GGGGS) 4 linker, and the VL region of the humanized and affinity matured anti-CD3 scFv domain was linked to the N-terminal of the CH2 domain of the generated antibody by a (GGGGS)₂ liker, the VH region of the humanized and affinity matured anti-CD3 scFv domain was linked to CH1 domain of one of the anti-GPRC5D Fab domains by a (GGGGS)₂ liker. LALA mutations (L234A, L235A, according to EU numbering) were introduced to each bispecific antibody to abolish the Fc receptor binding capability. ATG-Bis-40E9 prepared in Example 1 was used as the parental antibody control of these humanized bispecific antibodies. The resulting humanized 2+1 bispecific antibodies are referred to herein as Bis-40E9-L2H3-D56G.H, Bis-40E9-L2H7, Bis-40E9-L2H3-Y101F.H, Bis-40E9-L2H3-N55S.H, Bis-40E9-L2H3-D105R.H, Bis-40E9-L2H3, Bis-40E9-L2H9, Bis-40E9-L2H11, Bis-40E9-L5H7, Bis-40E9-L2H14, Bis-40E9-L2H6, Bis-40E9-L2H16, Bis-40E9-L2H10, wherein the prefix “Bis-” indicates “bispecific”, and the following indicates the humanized antibody from which the clone 40-C12-C10-E9 was derived. For example, “Bis-40E9-L2H7” indicates that it is a bispecific antibody with a Fab-scFv-Fab format, comprising a scFv (which comprises a humanized VL2 region and a humanized VH7 region from the hybridoma antibody clone 40-C12-C10-E9) targeting CD3 and a humanized Fab domain targeting GPRC5D (which comprises a VH region comprising an amino acid sequence as set forth in SEQ ID NO: 15 and a VL region comprising an amino acid sequence as set forth in SEQ ID NO: 18). The SEQ ID NOs and amino acid sequences of VL and VH regions of the scFv targeting CD3 in each of the generated humanized 2+1 bispecific antibodies (i.e., Bis-40E9-L2H3-D56G.H, Bis-40E9-L2H7, Bis-40E9-L2H3-Y101F.H, Bis-40E9-L2H3-N55S.H, Bis-40E9-L2H3-D105R.H, Bis-40E9-L2H3, Bis-40E9-L2H9, Bis-40E9-L2H11, Bis-40E9-L5H7, Bis-40E9-L2H14, Bis-40E9-L2H6, Bis-40E9-L2H16, Bis-40E9-L2H10) are shown in Table 36 and Table 37 below, and CDR sequences identified by the convention of Kabat are underlined in each sequence listed in Table 37.

TABLE 36
VH and VL Regions of the anti-CD3 scFv Domain of the Generated
Exemplary Bispecific Antibodies

| | VL Region | |
|---|---|---|
| | 40E9-VL2 | 40E9-VL5 |
| VH Region | (SEQ ID NO: 198) | (SEQ ID NO: 141) |
| 40E9-VH3 | 40E9-L2H3 | / |
| (SEQ ID NO: 119) | (SEQ ID NOs: 198/119) | |
| 40E9-VH3-N55S.H | 40E9-L2H3-N55S.H | / |
| (SEQ ID NO: 120) | (SEQ ID NOs: 198/120) | |
| 40E9-VH3-D56G.H | 40E9-L2H3-D56G.H | / |
| (SEQ ID NO: 122) | (SEQ ID NOs: 198/122) | |
| 40E9-VH3-Y101F.H | 40E9-L2H3-Y101F.H | / |
| (SEQ ID NO: 124) | (SEQ ID NOs: 198/124) | |
| 40E9-VH3-D105R.H | 40E9-L2H3-D105R.H | / |
| (SEQ ID NO: 125) | (SEQ ID NOs: 198/125) | |
| 40E9-VH6 | 40E9-L2H6 | / |
| (SEQ ID NO: 126) | (SEQ ID NOs: 198/126) | |
| 40E9-VH7 | 40E9-L2H7 | 40E9-L5H7 |
| (SEQ ID NO: 128) | (SEQ ID NOs: 198/128) | (SEQ ID NOs: 141/128) |
| 40E9-VH9 | 40E9-L2H9 | / |
| (SEQ ID NO: 131) | (SEQ ID NOs: 198/131) | |
| 40E9-VH10 | 40E9-L2H10 | / |
| (SEQ ID NO: 134) | (SEQ ID NOs: 198/134) | |
| 40E9-VH11 | 40E9-L2H11 | / |
| (SEQ ID NO: 136) | (SEQ ID NOs: 198/136) | |
| 40E9-VH14 | 40E9-L2H14 | / |
| (SEQ ID NO: 138) | (SEQ ID NOs: 198/138) | |
| 40E9-VH16 | 40E9-L2H16 | / |
| (SEQ ID NO: 139) | (SEQ ID NOs: 198/139) | |

TABLE 37
Amino Acid Sequences of VH and VL Regions of Humanized 40-C12-
C10-E9 Used in the Humanized 2+1 Bispecific Antibodies

| Description | SEQ ID NO: | Amino Acid Sequence |
|---|---|---|
| 40E9-VH3 | 119 | EVQLVQSGAEVKKPGSSVKVSCKASGYTFSSY |
| | | VIHWVRQAPGQCLEWMGYINPYNDVIKYNEK |
| | | FKGRVTLTSDKSTSTAYMELSSLRSEDTAVYY |
| | | CARDSYYSYDGFAYWGQGTLVTVS |
| 40E9-VH3- | 120 | EVQLVQSGAEVKKPGSSVKVSCKASGYTFSSY |
| N55S.H | | VIHWVRQAPGQCLEWMGYINPYSDVIKYNEK |
| | | FKGRVTLTSDKSTSTAYMELSSLRSEDTAVYY |
| | | CARDSYYSYDGFAYWGQGTLVTVSS |
| 40E9-VH3- | 122 | EVQLVQSGAEVKKPGSSVKVSCKASGYTFSSY |
| D56G.H | | VIHWVRQAPGQCLEWMGYINPYNGVIKYNE |
| | | KFKGRVTLTSDKSTSTAYMELSSLRSEDTAVY |
| | | YCARDSYYSYDGFAYWGQGTLVTVSS |
| 40E9-VH3- | 124 | EVQLVQSGAEVKKPGSSVKVSCKASGYTFSSY |
| Y101F.H | | VIHWVRQAPGQCLEWMGYINPYNDVIKYNEK |
| | | FKGRVTLTSDKSTSTAYMELSSLRSEDTAVYY |
| | | CARDSFYSYDGFAYWGQGTLVTVSS |
| 40E9-VH3- | 125 | EVQLVQSGAEVKKPGSSVKVSCKASGYTFSSY |
| D105R.H | | VIHWVRQAPGQCLEWMGYINPYNDVIKYNEK |
| | | FKGRVTLTSDKSTSTAYMELSSLRSEDTAVYY |
| | | CARDSYYSYRGFAYWGQGTLVTVSS |
| 40E9-VH6 | 126 | EVQLVQSGAEVKKPGSSVKVSCKASGYTFSSY |
| | | VIHWVRQAPGQCLEWMGYINPYNDVIKYNEK |
| | | FKGRVTLTSDKSTSTAYMELSSLRSEDTAVYY |
| | | CARESFSSYDGFAYWGQGTLVTVSS |
| 40E9-VH7 | 128 | EVQLVQSGAEVKKPGSSVKVSCKASGYTFSSY |
| | | VIHWVRQAPGQCLEWMGYINPGNGVIKYNE |
| | | KFKGRVTLTSDKSTSTAYMELSSLRSEDTAVY |
| | | YCARDSYSSYDGFAYWGQGTLVTVSS |
| 40E9-VH9 | 131 | EVQLVQSGAEVKKPGSSVKVSCKASGYTFSSY |
| | | VIHWVRQAPGQCLEWMGYINPYSDVIKYNEK |
| | | FKGRVTLTSDKSTSTAYMELSSLRSEDTAVYY |
| | | CARESFYSYDGFAYWGQGTLVTVSS |
| 40E9-VH10 | 134 | EVQLVQSGAEVKKPGSSVKVSCKASGYTFSSY |
| | | VIHWVRQAPGQCLEWMGYINPYNDVIKYNEK |
| | | FKGRVTLTSDKSTSTAYMELSSLRSEDTAVYY |
| | | CARDSYYSYEAFAYWGQGTLVTVSS |
| 40E9-VH11 | 136 | EVQLVQSGAEVKKPGSSVKVSCKASGYTFSSY |
| | | VIHWVRQAPGQCLEWMGYINPYNDVIKYNEK |
| | | FKGRVTLTSDKSTSTAYMELSSLRSEDTAVYY |
| | | CARDSYYSYRAFAYWGQGTLVTVSS |
| 40E9-VH14 | 138 | EVQLVQSGAEVKKPGSSVKVSCKASGYTFSSY |
| | | VIHWVRQAPGQCLEWMGYINPYNDVIKYNEK |
| | | FKGRVTLTSDKSTSTAYMELSSLRSEDTAVYY |
| | | CARESYYSYDGFAYWGQGTLVTVSS |
| 40E9-VH16 | 139 | EVQLVQSGAEVKKPGSSVKVSCKASGYTFSDY |
| | | VIHWVRQAPGQCLEWMGYINPYNDVIKYNEK |
| | | FKGRVTLTSDKSTSTAYMELSSLRSEDTAVYY |
| | | CARDSYYSYDGFAYWGQGTLVTVSS |
| 40E9-VL2 | 198 | EIVLTQSPDFQSVTPKEKVTITCSASSSVSYMH |
| | | WYQQKPDQSPKRLIYDTSKLASGVPSRFSGSG |
| | | SGTDFTLTINSLEAEDAATYYCQQWSSNPPTF |
| | | GCGTKVEIK |
| 40E9-VL5 | 141 | EIVLTQSPDFQSVTPKEKVTITCSASSSVSYMH |
| | | WYQQKPDQSPKRLIYDTSKLASGVPSRFSGSG |
| | | SGTDFTLTINSLEAEDAATYYCQQWRSSPPTF |
| | | GCGTKVEIK |

ATG-Bis-40E9 described in Example 1 was used as the parental antibody control of these antibodies. hIgG1 isotype was used as negative control.

### 6.2 Humanized 2+1 Bispecific Antibody Characterization: Binding Affinity to Jurkat Cells

The binding affinities of the generated humanized Fab-scFv-Fab bispecific antibodies were determined by FACS analysis. The protocol for FACS analysis is identical to that described in Example 3.2, except that different test antibodies were used.

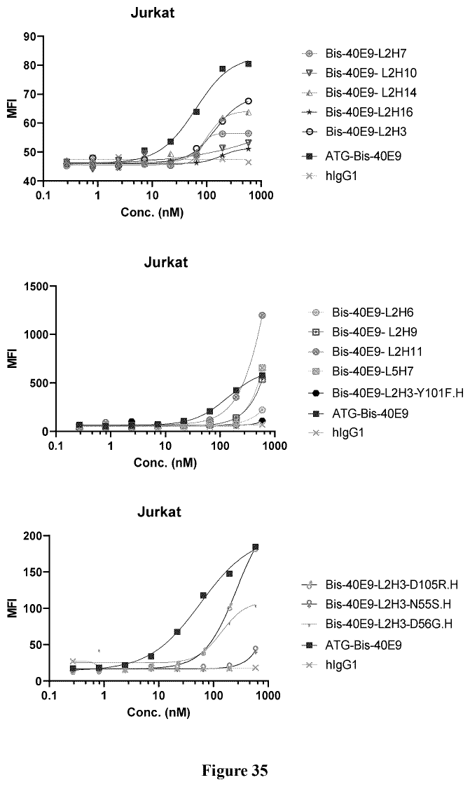

As shown in Table 38 and FIG. 35, the generated humanized Fab-scFv-Fab bispecific antibodies showed binding ability to Jurkat cells (as evidenced by higher EC₅₀ values) compared with parental antibodies.

TABLE 38
Ratio of EC₅₀ and MFI of Jurkat cell binding affinity of
humanized 2+1 bispecific antibodies on Jurkat

| | Jurkat cell binding | |
|---|---|---|
| | Ratio of EC₅₀ | Ratio of Top MFI |
| | (relative to | (relative to |
| Antibody | ATG-Bis-40E9) | ATG-Bis-40E9) |
| ATG-Bis-40E9 | 1 | 1 |
| Bis-40E9-L2H7 | 1.24 | 0.68 |
| Bis-40E9-L2H3 | 1.65 | 0.77 |
| Bis-40E9-L2H14 | 2.31 | 0.83 |
| Bis-40E9-L2H3-D56G.H | 2.41 | 0.55 |
| Bis-40E9-L2H16 | 2.97 | 0.62 |
| Bis-40E9-L2H3-D105R.H | 4.53 | 1.17 |
| Bis-40E9-L2H11 | 7.88 | 6.35 |
| Bis-40E9-L2H3-Y101F.H | 13.61 | Unstable |
| Bis-40E9-L2H3-N55S.H | 167.35 | Unstable |
| Bis-40E9-L5H7 | 210.11 | Unstable |
| Bis-40E9-L2H9 | 368.19 | 787.20 |
| Bis-40E9-L2H6 | 512.66 | Unstable |
| Bis-40E9-L2H10 | 27481.59 | 2.60 |

6.3 Humanized 2+1 Bispecific Antibody Characterization: Binding affinity to MM.1S

The binding affinities of the generated humanized Fab-scFv-Fab bispecific antibodies to MM.1S were evaluated by FACS analysis. The protocol for FACS analysis is identical to that described in Example 1.2, except that MM.1S were used in this example.

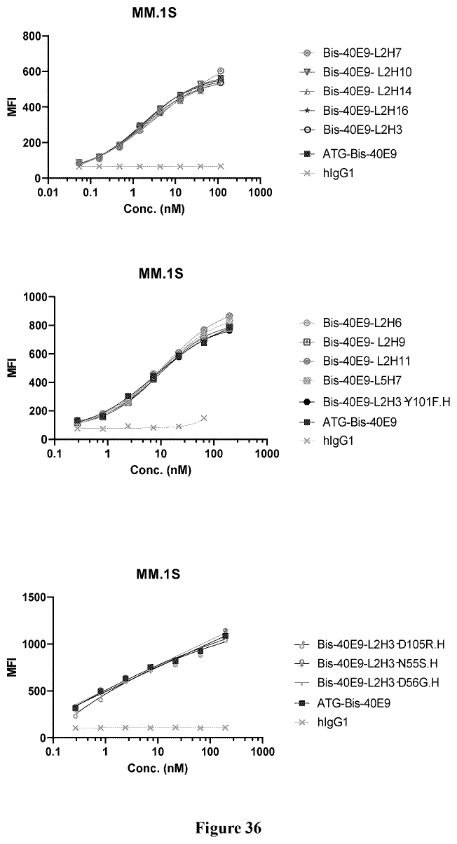

As shown in Table 39 and FIG. 36, the generated humanized Fab-scFv-Fab bispecific antibodies showed comparable EC₅₀ values on MM.1S compared to parental antibody ATG-Bis-40E9.

TABLE 39
Ratio of EC₅₀ and Top MFI of binding affinity of humanized
Fab-scFv-Fab bispecific antibody candidates on MM.1S

| | MM.1S cell binding | |
|---|---|---|
| | Ratio of EC₅₀ | Ratio of Top MFI |
| | (relative to | (relative to |
| Antibody | ATG-Bis-40E9) | ATG-Bis-40E9) |
| ATG-Bis-40E9 | 1 | 1 |
| Bis-40E9-L2H7 | 2.497 | 1.254 |
| Bis-40E9-L2H3 | 1.188 | 0.997 |
| Bis-40E9-L2H14 | 1.024 | 0.975 |
| Bis-40E9-L2H3-D56G.H | / | / |
| Bis-40E9-L2H16 | 0.913 | 0.972 |
| Bis-40E9-L2H3-D105R.H | / | / |
| Bis-40E9-L2H11 | 5.776 | 1.474 |
| Bis-40E9-L2H3-Y101F.H | 1.273 | 0.954 |
| Bis-40E9-L2H3-N55S.H | / | / |
| Bis-40E9-L5H7 | 1.138 | 0.989 |
| Bis-40E9-L2H9 | 0.926 | 0.874 |
| Bis-40E9-L2H6 | 0.824 | 0.877 |
| Bis-40E9-L2H10 | 1.145 | 1.022 |

### 6.4 Humanized 2+1 Bispecific Antibody Characterization: T-Cell Dependent Cytotoxicity

The cytotoxicity of humanized Fab-scFv-Fab bispecific antibodies on MM.1S cells and GPRC5D negative expressed cells (Daudi) were tested by FACS analysis. The protocol for FACS analysis is described as follows:
(a) CD3⁺ T cells were isolated from normal donors.
(b) 2×10⁴ target cells/well (CFSE-labeled MM.1S or Daudi) and 1×10⁵ effector cells/well (T cells) were added to wells of 96-well U-bottom plates (5:1 Effector: Target ratio).
(c) After mixing the target cells and T cells, dilutions of humanized 2+1 bispecific antibodies were added to each well (9 diluting concentrations from 5 nM, by 1:4 serial).
(d) The plates were incubated at 37° C. with 5% CO₂ overnight.
(e) After incubation, the plates were centrifuged and analyzed by FACS.

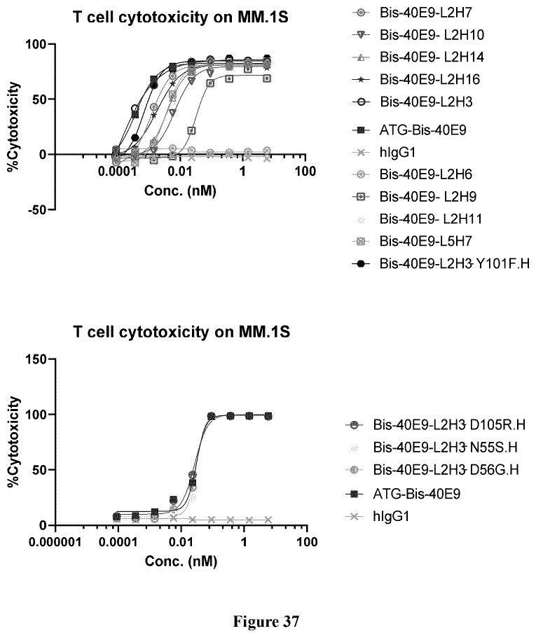

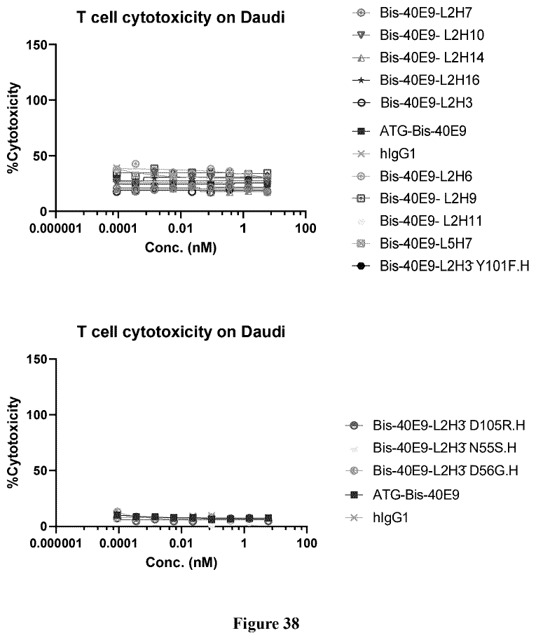

As shown in Table 40 and FIG. 37, the generated humanized Fab-scFv-Fab bispecific antibodies showed potent T cell dependent cytotoxicity on MM.1S cells. Antibodies showed 0.2-88 fold IC₅₀ values which indicated more or less potent T cell dependent cytotoxicity compared with parental antibody. In addition, as shown in FIG. 38, all of the generated humanized Fab-scFv-Fab bispecific antibodies had no non-specific T cell cytotoxicity.

TABLE 40
Ratio of IC₅₀ and Top target cells killing percentage of humanized
Fab-scFv-Fab bispecific antibodies induced by T-Cell
Dependent Cytotoxicity

| | MM.1S cytotoxicity | |
|---|---|---|
| | Ratio of IC₅₀ | Ratio of Top cytotoxicity |
| | (relative to | (relative to |
| Antibody | ATG-Bis-40E9) | ATG-Bis-40E9) |
| Bis-40E9-L2H3 | 0.23 | 1.04 |
| Bis-40E9-L2H3- | 0.86 | 1.01 |
| D105R.H | | |
| Bis-40E9-L2H3-D56G.H | 0.99 | 1.00 |
| ATG-Bis-40E9 | 1 | 1 |
| Bis-40E9-L2H3-N55S.H | 1.06 | 1.00 |
| Bis-40E9-L2H3- | 1.94 | 1.03 |
| Y101F.H | | |
| Bis-40E9-L2H7 | 3.21 | 0.98 |
| Bis-40E9-L2H16 | 4.93 | 0.98 |
| Bis-40E9-L5H7 | 9.82 | 0.99 |
| Bis-40E9-L2H14 | 10.38 | 0.98 |
| Bis-40E9-L2H11 | 13.65 | 1.00 |
| Bis-40E9-L2H10 | 18.40 | 0.96 |
| Bis-40E9-L2H9 | 87.71 | 0.87 |
| Bis-40E9-L2H6 | unstable | 0.06 |

6.5 Humanized 2+1 Bispecific Antibody Characterization: T Cell Activation with Jurkat NFAT Reporter Cell Line

The T cell activation effects of these generated humanized Fab-scFv-Fab bispecific antibodies were tested by Jurkat-NFAT-Luciferase assay, MM.1S cells and GPRC5D negative expressed Daudi cells were used as target cells and Jurkat was used as effector cell. The protocol is described as follows:
(a) Target and effector cells were harvested and re-suspended to be 2.5×10⁴ cells/well and 5×10⁴ cells/well, respectively.
(b) 5 nM antibodies were added to cells, incubated at 37° C., 5% CO₂ for 24 hours.
(c) Cells were centrifuged at 500 g for 10 minutes, and then the supernatants were discarded.
(d) 30 μL 1× lysis buffer (Promega RLB-E3971) was added to wells and incubated at −80° C. for 30 minutes.
(e) The plates were transferred into 37° C. incubator and incubated for 15 minutes to lysis cells. The lysates were spined at 500 g for 10 minutes and the supernatants were harvested for the test.
(f) 20 μl supernatant was transferred into 96-well white opaque plate, then mixed thoroughly with 100 μl Luciferase Assay Substrate. Luciferase signal was read by plate reader.

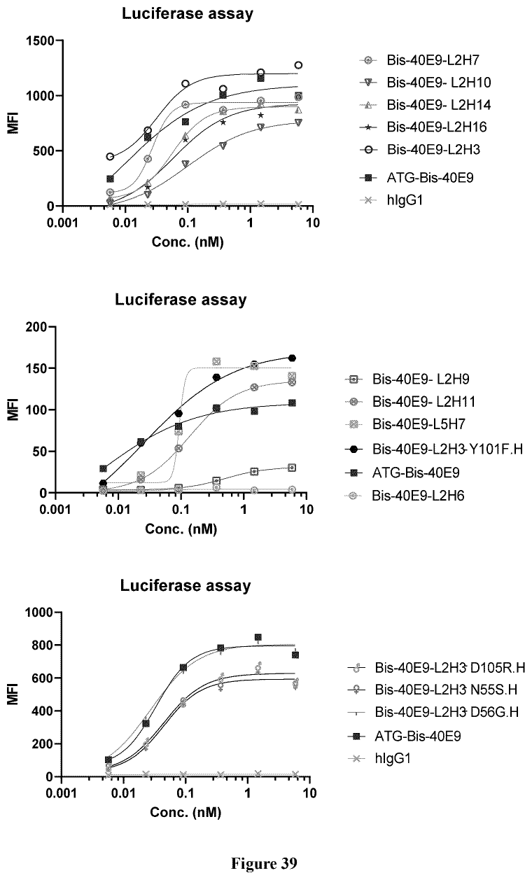

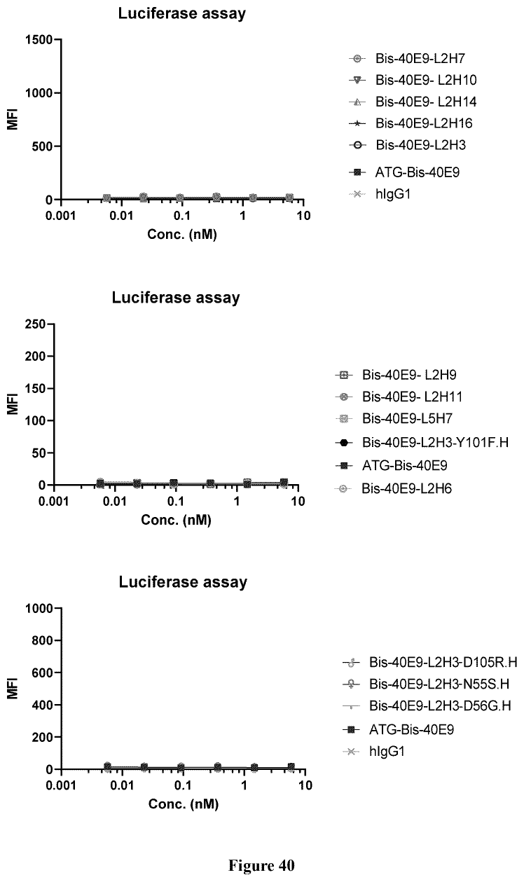

As shown in Table 41 and FIG. 39, the generated humanized Fab-scFv-Fab bispecific antibodies showed potent T cell activation on MM.1S, which was consistent with T cell cytotoxicity. In addition, as shown in FIG. 40, there was no non-specific activation on Daudi cells.

TABLE 41
Ratio of EC₅₀ and Top RLU of humanized Fab-scFv-Fab bispecific
antibodies induced by luciferase assay on MM.1S

| | Luciferase assay on MM.1S | |
|---|---|---|
| | Ratio of EC₅₀ | Ratio of Top RLU |
| | (relative to | (relative to |
| Antibody | ATG-Bis-40E9) | ATG-Bis-40E9) |
| Bis-40E9-L2H3-D56G.H | 0.81 | 1.01 |
| ATG-Bis-40E9 | 1 | 1 |
| Bis-40E9-L2H3-D105R.H | 1.32 | 0.79 |
| Bis-40E9-L2H3-N55S.H | 1.33 | 0.75 |
| Bis-40E9-L2H7 | 2.79 | 0.85 |
| Bis-40E9-L2H3 | 3.25 | 1.09 |
| Bis-40E9-L2H3-Y101F.H | 4.89 | 1.57 |
| Bis-40E9-L2H14 | 5.77 | 0.81 |
| Bis-40E9-L2H6 | unstable | 0.04 |
| Bis-40E9-L2H16 | 6.02 | 0.84 |
| Bis-40E9-L2H10 | 10.25 | 0.71 |
| Bis-40E9-L5H7 | 18.02 | 1.38 |
| Bis-40E9-L2H11 | 26.18 | 1.24 |
| Bis-40E9-L2H9 | 96.40 | 0.28 |

### Example 7. CD3 Bispecific Antibody Characterization: CD3 Antibody Binding Epitope

### 7.1 CD3 Antibody Binding Characteristics

### Methods

The inventors tested binding affinities of the anti-CD3 antibodies used in the instant invention (ch40E9, and ch40E9-derived humanized antibodies 40E9-L2H3 and 40E9-L2H3-N55S.H) and two benchmark CD3 antibodies including CD3 mAb-RG6234 and CD3 mAb-IO-312 that are derived from RG6234 (Roche) and IO-312 (Immune-Onc) analogues, respectively. The SEQ ID NOs of VH and VL regions of each antibody are shown in Table 42 below.

TABLE 42
SEQ ID NOs of VH and V regions of anti-CD3 antibodies

| | VH Region | VL Region |
|---|---|---|
| Antibody | (SEQ ID NO) | (SEQ ID NO) |
| ch40E9 | 3 | 4 |
| 40E9-L2H3 | 196 | 198 |
| 40E9-L2H3-N55S.H | 120 | 198 |
| CD3 mAb-RG6234 | 11 | 12 |
| CD3 mAb-IO-312 | 145 | 146 |

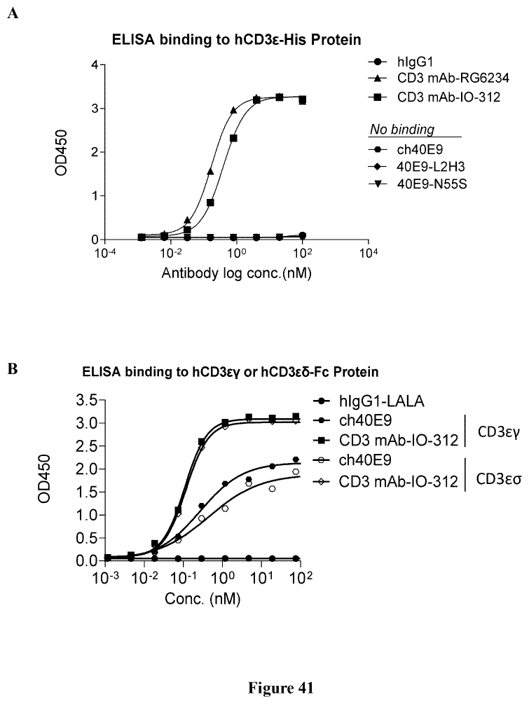

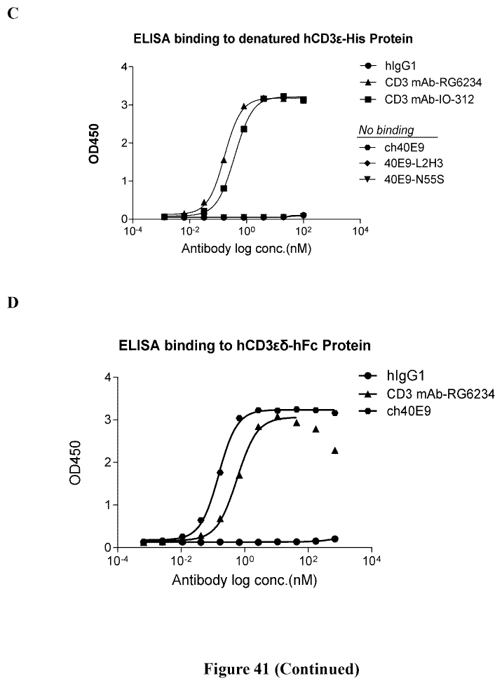

The binding affinity of these anti-CD3 antibodies with nature CD3ε, denatured CD3ε, CD3εγ/CD3εδ proteins detected by ELISA were showed in FIG. 41.

The protocol for ELISA assay is described as follows:
1. Added 100 μl/well diluted soluble protein (0.2 or 2 ug/mL) to Clear Flat-Bottom Immuno 96 well plate (Nunc, cat #442404). Incubate overnight at 4° C.
2. Washed 3 times with PBS containing 0.05% Tween-20 (PBST/BSA).
3. Blocked plates: 200 μl 2% BSA to each well. Incubated 1 hr at 37° C.
4. Washed 3 times with PBS containing 0.05% Tween-20 (PBST/BSA).
5. Added 100 μl test antibodies to each well. Incubated 1 hr at 37° C.
6. Washed 3 times with PBS containing 0.05% Tween-20 (PBST/BSA).
7. Added 100 μl HRP conjugated secondary antibody, dilution 1:10,000. Incubated 1 hr at 37° C.
8. Washed 3 times with PBS containing 0.05% Tween-20 (PBST/BSA).
9. Added 100 μl Substrate Solution TMB (SURMODICS, cat #TMBS-1000-01) to each well. Incubated 30 min RT in dark.
10. Added 50 μl Stop Solution to each well.
11. Read at 450 nm on MD SpectraMax iD3.

### Results

As shown in FIG. 41, CD3 mAb-RG6234 derived from RG6234 (Roche, WO2019154890A1) and CD3 mAb-IO-312 derived from IO-312 (Immune-Onc, WO2021183839A2) analogues bound to the CD3ε monomer (FIG. 41A). Reasonably, the two benchmarks also bound to the CD3εγ/CD3εδ dimer (FIG. 41B and D). More importantly, the two benchmarks bound to the CD3 monomer even after the CD3ε protein was denatured (FIG. 41C). These suggested that the two benchmarks CD3 mAb-RG6234 and CD3 mAb-IO-312 bound to a linear epitope of CD3ε monomer.

The anti-CD3 antibodies of the instant invention, including ch40E9, and ch40E9-derived humanized antibodies 40E9-L2H3 and 40E9-N55S, bound to the CD3εγ or CD3εδ dimer (FIG. 41B) and did not bind to the CD3ε monomer (FIG. 41A). This indicated that the anti-CD3 antibodies of the instant invention bound to the conformational epitope of CD3 protein.

Therefore, protein-based ELISA binding analysis suggested that these CD3 antibodies bound to different epitopes of CD3 protein.

### Example 8. CD3 Bispecific Antibody Characterization: TAA-dependent T Cell Activation

### 8.1 Methods

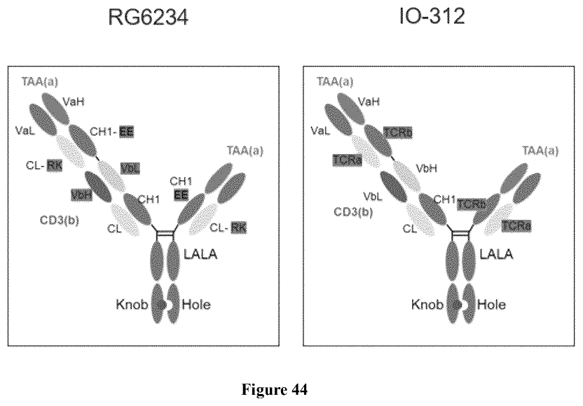

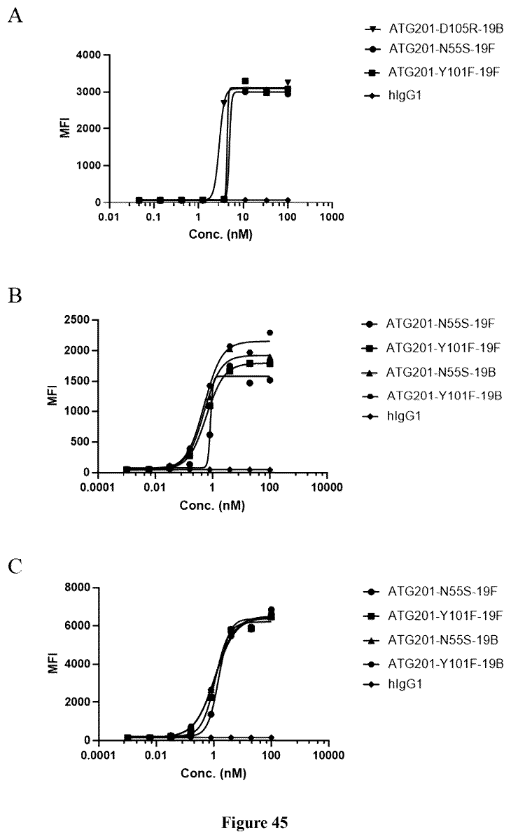

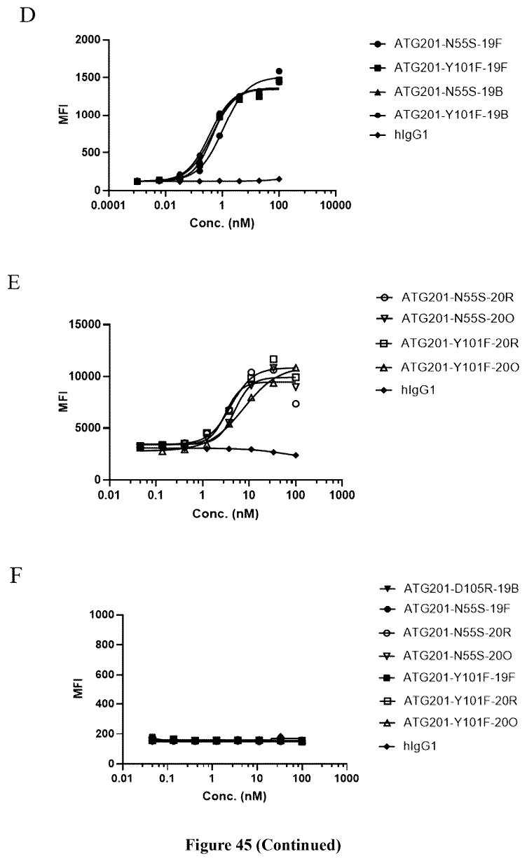

We produced different CD3-based bispecific antibodies, namely ATG-6234-1, ATG-6234-2, ATG-312-1, ATG-312-2, RG6234-N55S, RG6234-L2H3, IO-312-N55S and IO-312-L2H3 (Table 43) via introducing our anti-CD3 sequences 40E9-N55S and 40E9-L2H3 into RG6234 or IO-312 format (their illustrative constructs are shown in FIG. 44) or introducing anti-CD3 sequences (CD3 mAb-RG6234 from RG6234 and IO-312) into our Fab-scFv-Fab format (also named as “Antengager format” in the present disclosure).

TABLE 43
CD3 bispecific antibodies

| No. | BsAb | Format | CD3 | TAA |
|---|---|---|---|---|
| 1 | ATG6234-1 | Fab-scFv- | CD3 | GPRC5D |
| | | Fab | (RG6234) | (RG6234) |
| 2 | ATG6234-2 | Fab-scFv- | CD3 | GPRC5D |
| | | Fab | (RG6234) | (h108C4C12_VH3VL4) |
| 3 | ATG312-1 | Fab-scFv- | CD3 | LILRB4 |
| | | Fab | (IO-312) | (IO-312) |
| 4 | ATG312-2 | Fab-scFv- | CD3 | LILRB4 |
| | | Fab | (IO-312) | (PTMS16-22) |
| 5 | RG6234-N55S | RG6234 | 40E9-N55S | GPRC5D (RG6234) |
| 6 | RG6234-L2H3 | RG6234 | 40E9-L2H3 | GPRC5D (RG6234) |
| 7 | IO-312-N55S | IO-312 | 40E9-N55S | LILRB4 (IO-312) |
| 8 | IO-312-L2H3 | IO-312 | 40E9-L2H3 | LILRB4 (IO-312) |

The sequence information of each bispecific antibodies is shown in Table 44 below, and CDR sequences identified by the convention of Kabat are bold and underlined in each sequence.

TABLE 44
Sequence information of each CD3 bispecific antibodies

| | | Name of | | | |
|---|---|---|---|---|---|
| | | AA | | | SEQ |
| Name of | | se- | | | ID |
| antibody | | quence | Description | Sequence | NO: |
| CD3 | ch40E9 | ch40E9 | Parental | EVQLQLSGPELVKPGASVKMSCKA | 3 |
| mAb | | VH | mouse | SGYTFSSYVIHWVKQKPGQGLEWI | |
| | | | sequence | GYINPYNDVIKYNEKFKGKATLTS | |
| | | | | DKSSSTAYMELSSLTSEDSAVYYC | |
| | | | | ARDSYYSYDGFAYWGQGTLVTVS | |
| | | | | A | |
| | | ch40E9 | Parental | QIVLTQSPAIMSASPGEKVTMTCSA | 4 |
| | | VL | mouse | SSSVSYMHWYQQKSGTSPKRWIY | |
| | | | sequence | DTSKLASGVPARFSGSGSGTSYSLT | |
| | | | | ISSMEAEDAATYYCQQWSSNPPTF | |
| | | | | GGGTKVEIK | |
| CD3 | 40E | 40E9- | humanized | EVQLVQSGAEVKKPGSSVKVSCKA | 194 |
| mAb | 9- | N55S | 40E9 + | SGYTFSSYVIHWVRQAPGQGLEW | |
| | N55 | VH | affinity | MGYINPYSDVIKYNEKFKGRVTLT | |
| | S | | designed | SDKSTSTAYMELSSLRSEDTAVYY | |
| | | | (N55S) + | CARDSYYSYDGFAYWGQGTLVTV | |
| | | | back | SS | |
| | | | mutation | | |
| | | | (G27Y, I70L, | | |
| | | | A72S) | | |
| | | 40E9- | humanized | EIVLTQSPDFQSVTPKEKVTITCSAS | 24 |
| | | N55S | 40E9 + back | SSVSYMHWYQQKPDQSPKRLIYDT | |
| | | VL | mutation | SKLASGVPSRFSGSGSGTDFTLTINS | |
| | | | (L45R, K48Y) | LEAEDAATYYCQQWSSNPPTFGG | |
| | | | | GTKVEIK | |
| CD3 | 40E | 40E9- | humanized | EVQLVQSGAEVKKPGSSVKVSCKA | 22 |
| mAb | 9- | L2H3 | 40E9 + back | SGYTFSSYVIHWVRQAPGQGLEW | |
| | L2H | VH | mutation | MGYINPYNDVIKYNEKFKGRVTL | |
| | 3 | | (G27Y, I70L, | TSDKSTSTAYMELSSLRSEDTAVY | |
| | | | A72S) | YCARDSYYSYDGFAYWGQGTLVT | |
| | | | | VSS | |
| | | 40E9- | humanized | EIVLTQSPDFQSVTPKEKVTITCSAS | 24 |
| | | L2H3 | 40E9 + back | SSVSYMHWYQQKPDQSPKRLIYDT | |
| | | VL | mutation | SKLASGVPSRFSGSGSGTDFTLTINS | |
| | | | (L45R, K48Y) | LEAEDAATYYCQQWSSNPPTFGG | |
| | | | | GTKVEIK | |
| CD3 | CD3 | CD3- | CD3VH | EVQLLESGGGLVQPGGSLRLSCAA | 11 |
| mAb | mAb | RG6234 | sequence | SGFTFSTYAMNWVRQAPGKGLEW | |
| | - | VH | from RG6234 | VSRIRSKYNNYATYYADSVKGRF | |
| | RG6 | | | TISRDDSKNTLYLQMNSLRAEDTA | |
| | 234 | | | VYYCVRHGNFGNSYVSWFAYWG | |
| | | | | QGTLVTVSS | |
| | | CD3- | CD3VL | QAVVTQEPSLTVSPGGTVTLTCGSS | 12 |
| | | RG6234 | sequence | TGAVTTSNYANWVQEKPGQAFRG | |
| | | VL | from RG6234 | LIGGTNKRAPGTPARFSGSLLGGK | |
| | | | | AALTLSGAQPEDEAEYYCALWYS | |
| | | | | NLWVFGGGTKLTVL | |
| CD3 | CD3 | CD3-IO- | CD3 VH | QVQLVQSGAEVKKPGSSVKVSCKA | 145 |
| mAb | mAb | 312 VH | sequence | SGFAFTDYYIHWVRQAPGQGLEW | |
| | -IO- | | fromIQ-312 | MGWISPGNVNTKYNENFKGRVTI | |
| | 312 | | | TADKSTSTAYMELSSLRSEDTAVY | |
| | | | | YCARDGYSLYYFDYWGQGTLVTV | |
| | | | | SS | |
| | | CD3-IO- | CD3 VL | DIVMTQSPDSLAVSLGERATINCKS | 146 |
| | | 312 VL | sequence | SQSLLNSRTRKNYLAWYQQKPGQ | |
| | | | from IQ-312 | PPKLLIYWASTRQSGVPDRFSGSGS | |
| | | | | GTDFTLTISSLQAEDVAVYYCTQS | |
| | | | | HTLRTFGGGTKVEIK | |
| | | Human | Light chain | RTVAAPSVFIFPPSDEQLKSGTASV | 150 |
| | | kappa | constant | VCLLNNFYPREAKVQWKVDNALQ | |
| | | CL | region of | SGNSQESVTEQDSKDSTYSLSSTLT | |
| | | domain | mAb | LSKADYEKHKVYACEVTHQGLSSP | |
| | | | | VTKSFNRGEC | |
| | | Human | Heavy chain | ASTKGPSVFPLAPSSKSTSGGTAAL | 151 |
| | | IgG1 | constant | GCLVKDYFPEPVTVSWNSGALTSG | |
| | | heavy | region of | VHTFPAVLQSSGLYSLSSVVTVPSS | |
| | | chain | mAb + L234A, | SLGTQTYICNVNHKPSNTKVDKKV | |
| | | constant | L235A | EPKSCDKTHTCPPCPAPEAAGGPSV | |
| | | region | mutation | FLFPPKPKDTLMISRTPEVTCVVVD | |
| | | | | VSHEDPEVKFNWYVDGVEVHNAK | |
| | | | | TKPREEQYNSTYRVVSVLTVLHQD | |
| | | | | WLNGKEYKCKVSNKALPAPIEKTIS | |
| | | | | KAKGQPREPQVYTLPPSREEMTKN | |
| | | | | QVSLTCLVKGFYPSDIAVEWESNG | |
| | | | | QPENNYKTTPPVLDSDGSFFLYSKL | |
| | | | | TVDKSRWQQGNVFSCSVMHEALH | |
| | | | | NHYTQKSLSLSPGK | |
| GP | Bis- | Bis- | aGPRC5D | EVQLVQSGAEVKKPGASVKVSCK | 152 |
| RC5 | 40E | 40E9- | VH-IgG1 | ASGYTFTSYVIHWVRQAPGQGLE | |
| DxC | 9- | L2H3- | Hole | WMGYFNPYNDGTKYNEEFKGRV | |
| D3 | L2H | N55S.H_ | (L234A, | TLTSDKSTSTAYMELRSLRSDDTA | |
| BsAb | 3- | HC1 | L235A) | VYYCARGGVRRYFDVWGQGTTV | |
| | N55 | | | TVSS/ASTKGPSVFPLAPSSKSTSGG | |
| | S.H | | | TAALGCLVKDYFPEPVTVSWNSGA | |
| | | | | LTSGVHTFPAVLQSSGLYSLSSVVT | |
| | | | | VPSSSLGTQTYICNVNHKPSNTKVD | |
| | | | | KKVEPKSCDKTHTCPPCPAPEAAG | |
| | | | | GPSVFLFPPKPKDTLMISRTPEVTC | |
| | | | | VVVDVSHEDPEVKFNWYVDGVEV | |
| | | | | HNAKTKPREEQYNSTYRVVSVLTV | |
| | | | | LHQDWLNGKEYKCKVSNKALPAPI | |
| | | | | EKTISKAKGQPREPQVYTLPPCREE | |
| | | | | MTKNQVSLWCLVKGFYPSDIAVE | |
| | | | | WESNGQPENNYKTTPPVLDSDGSF | |
| | | | | FLYSKLTVDKSRWQQGNVFSCSV | |
| | | | | MHEALHNHYTQKSLSLSPGK | |
| | | Bis- | aGPRC5D | EVQLVQSGAEVKKPGASVKVSCK | 153 |
| | | 40E9- | VH-CH1- | ASGYTFTSYVIHWVRQAPGQGLE | |
| | | L2H3- | aCD3scFv | WMGYFNPYNDGTKYNEEFKGRVT | |
| | | N55S.H_ | (C-C)-IgG1 | LTSDKSTSTAYMELRSLRSDDTAV | |
| | | HC2 | Knob | YYCARGGVRRYFDVWGQGTTVT | |
| | | | (L234A, | VSS/ASTKGPSVFPLAPSSKSTSGGT | |
| | | | L235A) | AALGCLVKDYFPEPVTVSWNSGAL | |
| | | | | TSGVHTFPAVLQSSGLYSLSSVVTV | |
| | | | | PSSSLGTQTYICNVNHKPSNTKVDK | |
| | | | | KVEPKSC/GGGGSGGGGS/EVQLVQ | |
| | | | | SGAEVKKPGSSVKVSCKASGYTFS | |
| | | | | SYVIHWVRQAPGQCLEWMGYINP | |
| | | | | YSDVIKYNEKFKGRVTLTSDKSTST | |
| | | | | AYMELSSLRSEDTAVYYCARDSYY | |
| | | | | SYDGFAYWGQGTLVTVSS/GGGGS | |
| | | | | GGGGSGGGGSGGGGS/EIVLTQSPD | |
| | | | | FQSVTPKEKVTITCSASSSVSYMH | |
| | | | | WYQQKPDQSPKRLIYDTSKLASGV | |
| | | | | PSRFSGSGSGTDFTLTINSLEAEDAA | |
| | | | | TYYCQQWSSNPPTFGCGTKVEIK/ | |
| | | | | GGGGSGGGGS/DKTHTCPPCPAPEA | |
| | | | | AGGPSVFLFPPKPKDTLMISRTPEV | |
| | | | | TCVVVDVSHEDPEVKFNWYVDGV | |
| | | | | EVHNAKTKPREEQYNSTYRVVSVL | |
| | | | | TVLHQDWLNGKEYKCKVSNKALP | |
| | | | | APIEKTISKAKGQPREPQVCTLPPSR | |
| | | | | EEMTKNQVSLSCAVKGFYPSDIAV | |
| | | | | EWESNGQPENNYKTTPPVLDSDGS | |
| | | | | FFLVSKLTVDKSRWQQGNVFSCSV | |
| | | | | MHEALHNHYTQKSLSLSPGK | |
| | | Bis- | aGPRC5D | DIQMTQSPSSLSASVGDRVTITCRA | 154 |
| | | 40E9- | VL-CL | SQDIGSNLNWLQQKPGKAIKRLIY | |
| | | L2H3- | Kappa | ATFGLDSGVPSRFSGSRSGTDFTLT | |
| | | N55S.H_ | | ISSLQPEDFATYYCQQYASFPYTFG | |
| | | LC | | GGTKVEIK/RTVAAPSVFIFPPSDEQ | |
| | | | | LKSGTASVVCLLNNFYPREAKVQW | |
| | | | | KVDNALQSGNSQESVTEQDSKDST | |
| | | | | YSLSSTLTLSKADYEKHKVYACEV | |
| | | | | THQGLSSPVTKSFNRGEC | |
| GP | Bis- | Bis- | aGPRC5D | EVQLVQSGAEVKKPGASVKVSCK | 155 |
| RC5 | 40E | 40E9- | VH-IgG1 | ASGYTFTSYVIHWVRQAPGQGLE | |
| DxC | 9- | L2H3_H | Hole | WMGYFNPYNDGTKYNEEFKGRV | |
| | | C1 | (L234A, | TLTSDKSTSTAYMELRSLRSDDTA | |
| | | | L235A) | VYYCARGGVRRYFDVWGQGTTV | |
| | | | | TVSS/ASTKGPSVFPLAPSSKSTSGG | |
| | | | | TAALGCLVKDYFPEPVTVSWNSGA | |
| | | | | LTSGVHTFPAVLQSSGLYSLSSVVT | |
| | | | | VPSSSLGTQTYICNVNHKPSNTKVD | |
| | | | | KKVEPKSCDKTHTCPPCPAPEAAG | |
| | | | | GPSVFLFPPKPKDTLMISRTPEVTC | |
| | | | | VVVDVSHEDPEVKFNWYVDGVEV | |
| | | | | HNAKTKPREEQYNSTYRVVSVLTV | |
| | | | | LHQDWLNGKEYKCKVSNKALPAPI | |
| | | | | EKTISKAKGQPREPQVYTLPPCREE | |
| | | | | MTKNQVSLWCLVKGFYPSDIAVE | |
| | | | | WESNGQPENNYKTTPPVLDSDGSF | |
| | | | | FLYSKLTVDKSRWQQGNVFSCSV | |
| | | | | MHEALHNHYTQKSLSLSPGK | |
| D3 | L2H | Bis- | aGPRC5D | EVQLVQSGAEVKKPGASVKVSCK | 156 |
| BsA | 3 | 40E9- | VH-CH1- | ASGYTFTSYVIHWVRQAPGQGLE | |
| b | | L2H3_ | aCD3scFv | WMGYFNPYNDGTKYNEEFKGRVT | |
| | | HC2 | (C-C)-IgG1 | LTSDKSTSTAYMELRSLRSDDTA | |
| | | | Knob | VYYCARGGVRRYFDVWGQGTTVTV | |
| | | | (L234A, | SS/ASTKGPSVFPLAPSSKSTSGG | |
| | | | L235A) | TAALGCLVKDYFPEPVTVSWNSGA | |
| | | | | LTSGVHTFPAVLQSSGLYSLSSVVT | |
| | | | | VPSSSLGTQTYICNVNHKPSNTKVD | |
| | | | | KKVEPKSC/GGGGSGGGGS/EVQLV | |
| | | | | QSGAEVKKPGSSVKVSCKASGYTF | |
| | | | | SSYVIHWVRQAPGQCLEWMGYIN | |
| | | | | PYNDVIKYNEKFKGRVTLTSDKST | |
| | | | | STAYMELSSLRSEDTAVYYCARDS | |
| | | | | YYSYDGFAYWGQGTLVTVSS/GGG | |
| | | | | GSGGGGSGGGGSGGGGS/EIVLTQS | |
| | | | | PDFQSVTPKEKVTITCSASSSVSYM | |
| | | | | HWYQQKPDQSPKRLIYDTSKLASG | |
| | | | | VPSRFSGSGSGTDFTLTINSLEAED | |
| | | | | AATYYCQQWSSNPPTFGCGTKVEIK | |
| | | | | /GGGGSGGGGS/DKTHTCPPCPAPE | |
| | | | | AAGGPSVFLFPPKPKDTLMISRTPE | |
| | | | | VTCVVVDVSHEDPEVKFNWYVDG | |
| | | | | VEVHNAKTKPREEQYNSTYRVVSV | |
| | | | | LTVLHQDWLNGKEYKCKVSNKALP | |
| | | | | APIEKTISKAKGQPREPQVCTLPPS | |
| | | | | REEMTKNQVSLSCAVKGFYPSDIA | |
| | | | | VEWESNGQPENNYKTTPPVLDSDG | |
| | | | | SFFLVSKLTVDKSRWQQGNVFSCS | |
| | | | | VMHEALHNHYTQKSLSLSPGK | |
| | | Bis- | aGPRC5D | DIQMTQSPSSLSASVGDRVTITCRA | 154 |
| | | 40E9- | VL-CL | SQDIGSNLNWLQQKPGKAIKRLIY | |
| | | L2H3_L | Kappa | ATFGLDSGVPSRFSGSRSGTDFTLT | |
| | | C | | ISSLQPEDFATYYCQQYASFPYTFG | |
| | | | | GGTKVEIK/RTVAAPSVFIFPPSDEQ | |
| | | | | LKSGTASVVCLLNNFYPREAKVQW | |
| | | | | KVDNALQSGNSQESVTEQDSKDST | |
| | | | | YSLSSTLTLSKADYEKHKVYACEV | |
| | | | | THQGLSSPVTKSFNRGEC | |
| GP | RG6 | RG6234_ | aGPRC5D | EVQLVESGGGLVQPGRSLKLSCAA | 157 |
| RC5 | | HC1 | VH-CH1 | SGFSFSNYGMAWVRQAATKGLEW | |
| DxC | | | (EE)-IgG1Fc | VASISTGGGNTYYRDSVKGRFIVS | |
| | | | Hole | RDNAKNTQYLQMDSLRSEDTATY | |
| | | | (L234A, | YCTRHDRGGLYWGQGVMVTVSS/ | |
| | | | L235A, | ASTKGPSVFPLAPSSKSTSGGTAAL | |
| | | | P329G) | GCLVEDYFPEPVTVSWNSGALTSG | |
| | | | | VHTFPAVLQSSGLYSLSSVVTVPSS | |
| | | | | SLGTQTYICNVNHKPSNTKVDEKV | |
| | | | | EPKSCDKTHTCPPCPAPEAAGGPSV | |
| | | | | FLFPPKPKDTLMISRTPEVTCVVVD | |
| | | | | VSHEDPEVKFNWYVDGVEVHNAK | |
| | | | | TKPREEQYNSTYRVVSVLTVLHQD | |
| | | | | WLNGKEYKCKVSNKALGAPIEKTI | |
| | | | | SKAKGQPREPQVCTLPPSRDELTKN | |
| | | | | QVSLSCAVKGFYPSDIAVEWESNG | |
| | | | | QPENNYKTTPPVLDSDGSFFLVSKL | |
| | | | | TVDKSRWQQGNVFSCSVMHEALH | |
| | | | | NHYTQKSLSLSPGK | |
| D3 | 234 | RG6234_ | aGPRC5D | EVQLVESGGGLVQPGRSLKLSCAA | 158 |
| BsA | | HC2 | VH-CH1 | SGFSFSNYGMAWVRQAATKGLEW | |
| b | | | (EE)-aCD3 | VASISTGGGNTYYRDSVKGRFIVS | |
| | | | VL-IgG1 | RDNAKNTQYLQMDSLRSEDTATY | |
| | | | Knob | YCTRHDRGGLYWGQGVMVTVSS/ | |
| | | | (L234A, | ASTKGPSVFPLAPSSKSTSGGTAAL | |
| | | | L235A, | GCLVEDYFPEPVTVSWNSGALTSG | |
| | | | P329G) | VHTFPAVLQSSGLYSLSSVVTVPSS | |
| | | | | SLGTQTYICNVNHKPSNTKVDEKV | |
| | | | | EPKSCD/GGGGSGGGGS/QAVVTQE | |
| | | | | PSLTVSPGGTVTLTCGSSTGAVTTS | |
| | | | | NYANWVQEKPGQAFRGLIGGTNK | |
| | | | | RAPGTPARFSGSLLGGKAALTLSG | |
| | | | | AQPEDEAEYYCALWYSNLWVFGG | |
| | | | | GTKLTVL/SSASTKGPSVFPLAPSSK | |
| | | | | STSGGTAALGCLVKDYFPEPVTVS | |
| | | | | WNSGALTSGVHTFPAVLQSSGLYS | |
| | | | | LSSVVTVPSSSLGTQTYICNVNHKP | |
| | | | | SNTKVDKKVEPKSCDKTHTCPPCP | |
| | | | | APEAAGGPSVFLFPPKPKDTLMISR | |
| | | | | TPEVTCVVVDVSHEDPEVKFNWY | |
| | | | | VDGVEVHNAKTKPREEQYNSTYR | |
| | | | | VVSVLTVLHQDWLNGKEYKCKVS | |
| | | | | NKALGAPIEKTISKAKGQPREPQVY | |
| | | | | TLPPCRDELTKNQVSLWCLVKGFY | |
| | | | | PSDIAVEWESNGQPENNYKTTPPVL | |
| | | | | DSDGSFFLYSKLTVDKSRWQQGNV | |
| | | | | FSCSVMHEALHNHYTQKSLSLSPG | |
| | | | | K | |
| | | RG6234_ | aGPRC5D | DIVMTQAPLSVSVTPGESASISCRSS | 159 |
| | | LC1 | VL-CL | KSLLHSNGITYVYWYFQKPGKSPQ | |
| | | | Kappa(RK) | VLIYRMSNLASGVPDRFSGSGSET | |
| | | | | DFTLKISRVEAEDVGIYHCGQLLE | |
| | | | | NPYTFGAGTELELK/RTVAAPSVFIF | |
| | | | | PPSDRKLKSGTASVVCLLNNFYPRE | |
| | | | | AKVQWKVDNALQSGNSQESVTEQ | |
| | | | | DSKDSTYSLSSTLTLSKADYEKHK | |
| | | | | VYACEVTHQGLSSPVTKSFNRGEC | |
| | | RG6234_ | aCD3VH- | EVQLLESGGGLVQPGGSLRLSCAA | 160 |
| | | LC2 | CLKappa | SGFTFSTYAMNWVRQAPGKGLEW | |
| | | | | VSRIRSKYNNYATYYADSVKGRF | |
| | | | | TISRDDSKNTLYLQMNSLRAEDTA | |
| | | | | VYYCVRHGNFGNSYVSWFAYWG | |
| | | | | QGTLVTVSS/RTVAAPSVFIFPPSDE | |
| | | | | QLKSGTASVVCLLNNFYPREAKVQ | |
| | | | | WKVDNALQSGNSQESVTEQDSKD | |
| | | | | STYSLSSTLTLSKADYEKHKVYAC | |
| | | | | EVTHQGLSSPVTKSFNRGEC | |
| GP | AT | ATG623 | aGPRC5D | EVQLVESGGGLVQPGRSLKLSCAA | 161 |
| RC5 | G62 | 4-1_HC1 | VH-IgG1 | SGFSFSNYGMAWVRQAATKGLEW | |
| DxC | 34-1 | | Hole | VASISTGGGNTYYRDSVKGRFIVS | |
| D3 | | | (L234A, | RDNAKNTQYLQMDSLRSEDTATY | |
| BsA | | | L235A) | YCTRHDRGGLYWGQGVMVTVSS/ | |
| b | | | | ASTKGPSVFPLAPSSKSTSGGTAAL | |
| | | | | GCLVKDYFPEPVTVSWNSGALTSG | |
| | | | | VHTFPAVLQSSGLYSLSSVVTVPSS | |
| | | | | SLGTQTYICNVNHKPSNTKVDKKV | |
| | | | | EPKSCDKTHTCPPCPAPEAAGGPSV | |
| | | | | FLFPPKPKDTLMISRTPEVTCVVVD | |
| | | | | VSHEDPEVKFNWYVDGVEVHNAK | |
| | | | | TKPREEQYNSTYRVVSVLTVLHQD | |
| | | | | WLNGKEYKCKVSNKALPAPIEKTIS | |
| | | | | KAKGQPREPQVYTLPPCREEMTKN | |
| | | | | QVSLWCLVKGFYPSDIAVEWESNG | |
| | | | | QPENNYKTTPPVLDSDGSFFLYSKL | |
| | | | | TVDKSRWQQGNVFSCSVMHEALH | |
| | | | | NHYTQKSLSLSPGK | |
| | | ATG623 | aGPRC5D | EVQLVESGGGLVQPGRSLKLSCAA | 162 |
| | | 4-1_HC2 | VH-CH1- | SGFSFSNYGMAWVRQAATKGLEW | |
| | | | aCD3scFv | VASISTGGGNTYYRDSVKGRFIVS | |
| | | | (C-C)-IgG1 | RDNAKNTQYLQMDSLRSEDTATY | |
| | | | Knob | YCTRHDRGGLYWGQGVMVTVSS | |
| | | | (L234A, | /ASTKGPSVFPLAPSSKSTSGGTAAL | |
| | | | L235A) | GCLVKDYFPEPVTVSWNSGALTSG | |
| | | | | VHTFPAVLQSSGLYSLSSVVTVPSS | |
| | | | | SLGTQTYICNVNHKPSNTKVDKKV | |
| | | | | EPKSC/GGGGSGGGGS/EVQLLESG | |
| | | | | GGLVQPGGSLRLSCAASGFTFSTY | |
| | | | | AMNWVRQAPGKCLEWVSRIRSKY | |
| | | | | NNYATYYADSVKGRFTISRDDSKN | |
| | | | | TLYLQMNSLRAEDTAVYYCVRHG | |
| | | | | NFGNSYVSWFAYWGQGTLVTVSS/ | |
| | | | | GGGGSGGGGSGGGGSGGGGS/QAV | |
| | | | | VTQEPSLTVSPGGTVTLTCGSSTGA | |
| | | | | VTTSNYANWVQEKPGQAFRGLIG | |
| | | | | GTNKRAPGTPARFSGSLLGGKAAL | |
| | | | | TLSGAQPEDEAEYYCALWYSNLW | |
| | | | | VFGCGTKLTVL/GGGGSGGGGS/DK | |
| | | | | THTCPPCPAPEAAGGPSVFLFPPKP | |
| | | | | KDTLMISRTPEVTCVVVDVSHEDP | |
| | | | | EVKFNWYVDGVEVHNAKTKPREE | |
| | | | | QYNSTYRVVSVLTVLHQDWLNGK | |
| | | | | EYKCKVSNKALPAPIEKTISKAKGQ | |
| | | | | PREPQVCTLPPSREEMTKNQVSLSC | |
| | | | | AVKGFYPSDIAVEWESNGQPENNY | |
| | | | | KTTPPVLDSDGSFFLVSKLTVDKSR | |
| | | | | WQQGNVFSCSVMHEALHNHYTQK | |
| | | | | SLSLSPGK | |
| | | ATG623 | aGPRC5D | DIVMTQAPLSVSVTPGESASISCRSS | 163 |
| | | 4-1 LC | VL-CL | KSLLHSNGITYVYWYFQKPGKSPQ | |
| | | | Kappa | VLIYRMSNLASGVPDRFSGSGSET | |
| | | | | DFTLKISRVEAEDVGIYHCGQLLE | |
| | | | | NPYTFGAGTELELK/RTVAAPSVFIF | |
| | | | | PPSDEQLKSGTASVVCLLNNFYPRE | |
| | | | | AKVQWKVDNALQSGNSQESVTEQ | |
| | | | | DSKDSTYSLSSTLTLSKADYEKHK | |
| | | | | VYACEVTHQGLSSPVTKSFNRGEC | |
| GP | AT | ATG623 | aGPRC5D | EVQLVQSGAEVKKPGASVKVSCK | 164 |
| RC5 | G62 | 4-2 HC1 | VH-IgG1 | ASGYTFTSYVIHWVRQAPGQGLE | |
| DxC | 34-2 | | Hole | WMGYFNPYNDGTKYNEEFKGRV | |
| D3 | | | (L234A, | TLTSDKSTSTAYMELRSLRSDDTA | |
| BsA | | | L235A) | VYYCARGGVRRYFDVWGQGTTV | |
| b | | | | TVSS/ASTKGPSVFPLAPSSKSTSGG | |
| | | | | TAALGCLVKDYFPEPVTVSWNSGA | |
| | | | | LTSGVHTFPAVLQSSGLYSLSSVVT | |
| | | | | VPSSSLGTQTYICNVNHKPSNTKVD | |
| | | | | KKVEPKSCDKTHTCPPCPAPEAAG | |
| | | | | GPSVFLFPPKPKDTLMISRTPEVTC | |
| | | | | VVVDVSHEDPEVKFNWYVDGVEV | |
| | | | | HNAKTKPREEQYNSTYRVVSVLTV | |
| | | | | LHQDWLNGKEYKCKVSNKALPAPI | |
| | | | | EKTISKAKGQPREPQVYTLPPCREE | |
| | | | | MTKNQVSLWCLVKGFYPSDIAVE | |
| | | | | WESNGQPENNYKTTPPVLDSDGSF | |
| | | | | FLYSKLTVDKSRWQQGNVFSCSV | |
| | | | | MHEALHNHYTQKSLSLSPGK | |
| | | ATG623 | aGPRC5D | EVQLVQSGAEVKKPGASVKVSCK | 165 |
| | | 4-2 HC2 | VH-CH1- | ASGYTFTSYVIHWVRQAPGQGLE | |
| | | | aCD3scFv | WMGYFNPYNDGTKYNEEFKGRV | |
| | | | (C-C)-IgG1 | TLTSDKSTSTAYMELRSLRSDDTA | |
| | | | Knob | VYYCARGGVRRYFDVWGQGTTV | |
| | | | (L234A, | TVSS/ASTKGPSVFPLAPSSKSTSGG | |
| | | | L235A) | TAALGCLVKDYFPEPVTVSWNSGA | |
| | | | | LTSGVHTFPAVLQSSGLYSLSSVVT | |
| | | | | VPSSSLGTQTYICNVNHKPSNTKVD | |
| | | | | KKVEPKSC/GGGGSGGGGS/EVQLL | |
| | | | | ESGGGLVQPGGSLRLSCAASGFTFS | |
| | | | | TYAMNWVRQAPGKCLEWVSRIRS | |
| | | | | KYNNYATYYADSVKGRFTISRDDS | |
| | | | | KNTLYLQMNSLRAEDTAVYYCVR | |
| | | | | HGNFGNSYVSWFAYWGQGTLVT | |
| | | | | VSS/GGGGSGGGGSGGGGSGGGGS/ | |
| | | | | QAVVTQEPSLTVSPGGTVTLTCGSS | |
| | | | | TGAVTTSNYANWVQEKPGQAFRG | |
| | | | | LIGGTNKRAPGTPARFSGSLLGGK | |
| | | | | AALTLSGAQPEDEAEYYCALWYS | |
| | | | | NLWVFGCGTKLTVL/GGGGSGGG | |
| | | | | GS/DKTHTCPPCPAPEAAGGPSVFL | |
| | | | | FPPKPKDTLMISRTPEVTCVVVDVS | |
| | | | | HEDPEVKFNWYVDGVEVHNAKTK | |
| | | | | PREEQYNSTYRVVSVLTVLHQDWL | |
| | | | | NGKEYKCKVSNKALPAPIEKTISKA | |
| | | | | KGQPREPQVCTLPPSREEMTKNQV | |
| | | | | SLSCAVKGFYPSDIAVEWESNGQPE | |
| | | | | NNYKTTPPVLDSDGSFFLVSKLTVD | |
| | | | | KSRWQQGNVFSCSVMHEALHNHY | |
| | | | | TQKSLSLSPGK | |
| | | ATG623 | aGPRC5D | DIQMTQSPSSLSASVGDRVTITCRA | 154 |
| | | 4-2 LC | VL-CL | SQDIGSNLNWLQQKPGKAIKRLIY | |
| | | | Kappa | ATFGLDSGVPSRFSGSRSGTDFTLT | |
| | | | | ISSLQPEDFATYYCQQYASFPYTFG | |
| | | | | GGTKVEIK/RTVAAPSVFIFPPSDEQ | |
| | | | | LKSGTASVVCLLNNFYPREAKVQW | |
| | | | | KVDNALQSGNSQESVTEQDSKDST | |
| | | | | YSLSSTLTLSKADYEKHKVYACEV | |
| | | | | THQGLSSPVTKSFNRGEC | |
| GP | RG6 | RG6234- | aGPRC5D | EVQLVESGGGLVQPGRSLKLSCAA | 166 |
| RC5 | 234- | N55S H | VH-CH1 | SGFSFSNYGMAWVRQAATKGLEW | |
| DxC | | C1 | (EE)-IgG1Fc | VASISTGGGNTYYRDSVKGRFIVS | |
| | | | Hole | RDNAKNTQYLQMDSLRSEDTATY | |
| | | | (L234A, | YCTRHDRGGLYWGQGVMVTVSS/ | |
| | | | L235A, | ASTKGPSVFPLAPSSKSTSGGTAAL | |
| | | | P329G) | GCLVEDYFPEPVTVSWNSGALTSG | |
| | | | | VHTFPAVLQSSGLYSLSSVVTVPSS | |
| | | | | SLGTQTYICNVNHKPSNTKVDEKV | |
| | | | | EPKSCDKTHTCPPCPAPEAAGGPSV | |
| | | | | FLFPPKPKDTLMISRTPEVTCVVVD | |
| | | | | VSHEDPEVKFNWYVDGVEVHNAK | |
| | | | | TKPREEQYNSTYRVVSVLTVLHQD | |
| | | | | WLNGKEYKCKVSNKALGAPIEKTI | |
| | | | | SKAKGQPREPQVCTLPPSRDELTKN | |
| | | | | QVSLSCAVKGFYPSDIAVEWESNG | |
| | | | | QPENNYKTTPPVLDSDGSFFLVSKL | |
| | | | | TVDKSRWQQGNVFSCSVMHEALH | |
| | | | | NHYTQKSLSLSPGK | |
| D3 | N55 | RG6234- | aGPRC5D | EVQLVESGGGLVQPGRSLKLSCAA | 167 |
| BsA | S | N55S H | VH-CH1 | SGFSFSNYGMAWVRQAATKGLEW | |
| b | | C2 | (EE)-aCD3 | VASISTGGGNTYYRDSVKGRFIVS | |
| | | | VL-IgG1 | RDNAKNTQYLQMDSLRSEDTATY | |
| | | | Knob | YCTRHDRGGLYWGQGVMVTVSS/ | |
| | | | (L234A, | ASTKGPSVFPLAPSSKSTSGGTAAL | |
| | | | L235A, | GCLVEDYFPEPVTVSWNSGALTSG | |
| | | | P329G) | VHTFPAVLQSSGLYSLSSVVTVPSS | |
| | | | | SLGTQTYICNVNHKPSNTKVDEKV | |
| | | | | EPKSCD/GGGGSGGGGS/EIVLTQSP | |
| | | | | DFQSVTPKEKVTITCSASSSVSYMH | |
| | | | | WYQQKPDQSPKRLIYDTSKLASGV | |
| | | | | PSRFSGSGSGTDFTLTINSLEAEDAA | |
| | | | | TYYCQQWSSNPPTFGGGTKVEIK/ | |
| | | | | SSASTKGPSVFPLAPSSKSTSGGTA | |
| | | | | ALGCLVKDYFPEPVTVSWNSGALT | |
| | | | | SGVHTFPAVLQSSGLYSLSSVVTVP | |
| | | | | SSSLGTQTYICNVNHKPSNTKVDK | |
| | | | | KVEPKSCDKTHTCPPCPAPEAAGG | |
| | | | | PSVFLFPPKPKDTLMISRTPEVTCV | |
| | | | | VVDVSHEDPEVKFNWYVDGVEVH | |
| | | | | NAKTKPREEQYNSTYRVVSVLTVL | |
| | | | | HQDWLNGKEYKCKVSNKALGAPI | |
| | | | | EKTISKAKGQPREPQVYTLPPCRDE | |
| | | | | LTKNQVSLWCLVKGFYPSDIAVEW | |
| | | | | ESNGQPENNYKTTPPVLDSDGSFFL | |
| | | | | YSKLTVDKSRWQQGNVFSCSVMH | |
| | | | | EALHNHYTQKSLSLSPGK | |
| | | RG6234- | aGPRC5D | DIVMTQAPLSVSVTPGESASISCRSS | 159 |
| | | N55S_L | VL-CL | KSLLHSNGITYVYWYFQKPGKSPQ | |
| | | C1 | Kappa(RK) | VLIYRMSNLASGVPDRFSGSGSET | |
| | | | | DFTLKISRVEAEDVGIYHCGQLLE | |
| | | | | NPYTFGAGTELELK/RTVAAPSVFIF | |
| | | | | PPSDRKLKSGTASVVCLLNNFYPRE | |
| | | | | AKVQWKVDNALQSGNSQESVTEQ | |
| | | | | DSKDSTYSLSSTLTLSKADYEKHK | |
| | | | | VYACEVTHQGLSSPVTKSFNRGEC | |
| | | RG6234- | aCD3VH- | EVQLVQSGAEVKKPGSSVKVSCKA | 168 |
| | | N55S L | CLKappa | SGYTFSSYVIHWVRQAPGQGLEW | |
| | | C2 | | MGYINPYSDVIKYNEKFKGRVTLT | |
| | | | | SDKSTSTAYMELSSLRSEDTAVYY | |
| | | | | CARDSYYSYDGFAYWGQGTLVTV | |
| | | | | SS/RTVAAPSVFIFPPSDEQLKSGTA | |
| | | | | SVVCLLNNFYPREAKVQWKVDNA | |
| | | | | LQSGNSQESVTEQDSKDSTYSLSST | |
| | | | | LTLSKADYEKHKVYACEVTHQGLS | |
| | | | | SPVTKSFNRGEC | |
| GP | RG6 | RG6234- | aGPRC5D | EVQLVESGGGLVQPGRSLKLSCAA | 169 |
| RC5 | 234- | L2H3 H | VH-CH1 | SGFSFSNYGMAWVRQAATKGLEW | |
| DxC | | C1 | (EE)-IgG1Fc | VASISTGGGNTYYRDSVKGRFIVS | |
| | | | Hole | RDNAKNTQYLQMDSLRSEDTATY | |
| | | | (L234A, | YCTRHDRGGLYWGQGVMVTVSS/ | |
| | | | L235A, | ASTKGPSVFPLAPSSKSTSGGTAAL | |
| | | | P329G) | GCLVEDYFPEPVTVSWNSGALTSG | |
| | | | | VHTFPAVLQSSGLYSLSSVVTVPSS | |
| | | | | SLGTQTYICNVNHKPSNTKVDEKV | |
| | | | | EPKSCDKTHTCPPCPAPEAAGGPSV | |
| | | | | FLFPPKPKDTLMISRTPEVTCVVVD | |
| | | | | VSHEDPEVKFNWYVDGVEVHNAK | |
| | | | | TKPREEQYNSTYRVVSVLTVLHQD | |
| | | | | WLNGKEYKCKVSNKALGAPIEKTI | |
| | | | | SKAKGQPREPQVCTLPPSRDELTKN | |
| | | | | QVSLSCAVKGFYPSDIAVEWESNG | |
| | | | | QPENNYKTTPPVLDSDGSFFLVSKL | |
| | | | | TVDKSRWQQGNVFSCSVMHEALH | |
| | | | | NHYTQKSLSLSPGK | |
| D3 | L2H | RG6234- | aGPRC5D | EVQLVESGGGLVQPGRSLKLSCAA | 170 |
| BsA | 3 | L2H3 H | VH-CH1 | SGFSFSNYGMAWVRQAATKGLEW | |
| b | | C2 | (EE)-aCD3 | VASISTGGGNTYYRDSVKGRFIVS | |
| | | | VL-IgG1 | RDNAKNTQYLQMDSLRSEDTATY | |
| | | | Knob | YCTRHDRGGLYWGQGVMVTVSS/ | |
| | | | (L234A, | ASTKGPSVFPLAPSSKSTSGGTAAL | |
| | | | L235A, | GCLVEDYFPEPVTVSWNSGALTSG | |
| | | | P329G) | VHTFPAVLQSSGLYSLSSVVTVPSS | |
| | | | | SLGTQTYICNVNHKPSNTKVDEKV | |
| | | | | EPKSCD/GGGGSGGGGS/EIVLTQSP | |
| | | | | DFQSVTPKEKVTITCSASSSVSYMH | |
| | | | | WYQQKPDQSPKRLIYDTSKLASGV | |
| | | | | PSRFSGSGSGTDFTLTINSLEAEDAA | |
| | | | | TYYCQQWSSNPPTFGGGTKVEIK/ | |
| | | | | SSASTKGPSVFPLAPSSKSTSGGTA | |
| | | | | ALGCLVKDYFPEPVTVSWNSGALT | |
| | | | | SGVHTFPAVLQSSGLYSLSSVVTVP | |
| | | | | SSSLGTQTYICNVNHKPSNTKVDK | |
| | | | | KVEPKSCDKTHTCPPCPAPEAAGG | |
| | | | | PSVFLFPPKPKDTLMISRTPEVTCV | |
| | | | | VVDVSHEDPEVKFNWYVDGVEVH | |
| | | | | NAKTKPREEQYNSTYRVVSVLTVL | |
| | | | | HQDWLNGKEYKCKVSNKALGAPI | |
| | | | | EKTISKAKGQPREPQVYTLPPCRDE | |
| | | | | LTKNQVSLWCLVKGFYPSDIAVEW | |
| | | | | ESNGQPENNYKTTPPVLDSDGSFFL | |
| | | | | YSKLTVDKSRWQQGNVFSCSVMH | |
| | | | | EALHNHYTQKSLSLSPGK | |
| | | RG6234- | aGPRC5D | DIVMTQAPLSVSVTPGESASISCRSS | 159 |
| | | L2H3 L | VL-CL | KSLLHSNGITYVYWYFQKPGKSPQ | |
| | | C1 | Kappa(RK) | VLIYRMSNLASGVPDRFSGSGSET | |
| | | | | DFTLKISRVEAEDVGIYHCGQLLE | |
| | | | | NPYTFGAGTELELK/RTVAAPSVFIF | |
| | | | | PPSDRKLKSGTASVVCLLNNFYPRE | |
| | | | | AKVQWKVDNALQSGNSQESVTEQ | |
| | | | | DSKDSTYSLSSTLTLSKADYEKHK | |
| | | | | VYACEVTHQGLSSPVTKSFNRGEC | |
| | | RG6234- | aCD3VH- | EVQLVQSGAEVKKPGSSVKVSCKA | 171 |
| | | L2H3 L | CLKappa | SGYTFSSYVIHWVRQAPGQGLEW | |
| | | C2 | | MGYINPYNDVIKYNEKFKGRVTL | |
| | | | | TSDKSTSTAYMELSSLRSEDTAVY | |
| | | | | YCARDSYYSYDGFAYWGQGTLVT | |
| | | | | VSS/RTVAAPSVFIFPPSDEQLKSGT | |
| | | | | ASVVCLLNNFYPREAKVQWKVDN | |
| | | | | ALQSGNSQESVTEQDSKDSTYSLSS | |
| | | | | TLTLSKADYEKHKVYACEVTHQGL | |
| | | | | SSPVTKSFNRGEC | |
| LIL | IO- | IO- | aLILRB4 | EVQLVESGGGLVQPGGSLRLSCAA | 172 |
| RB4 | 312 | 312 HC | VH-TCRβ- | SGFSLSSSYWISWVRQAPGKGLEW | |
| xCD | | 1 | aCD3VH- | IGSIDSGSVGITYYATWVKGRFTIS | |
| 3 | | | IgG1Knob | RDNSKNTLYLQMNSLRAEDTAVY | |
| BsA | | | (L234A, | YCARHGDNWALDLWGQGTLVTV | |
| b | | | L235A) | LE/DLKNVFPPEVAVFEPSEAEISHT | |
| | | | | QKATLVCLATGFYPDHVELSWWV | |
| | | | | NGKEVHSGVCTDPQPLKEQPALQD | |
| | | | | SRYALSSRLRVSATFWQNPRNHFR | |
| | | | | CQVQFYGLSENDEWTQDRAKPVT | |
| | | | | QIVSAEAWGR/GGGGSGGGGS/QV | |
| | | | | QLVQSGAEVKKPGSSVKVSCKASG | |
| | | | | FAFTDYYIHWVRQAPGQGLEWMG | |
| | | | | WISPGNVNTKYNENFKGRVTITA | |
| | | | | DKSTSTAYMELSSLRSEDTAVYYC | |
| | | | | ARDGYSLYYFDYWGQGTLVTVSS/ | |
| | | | | ASTKGPSVFPLAPSSKSTSGGTAAL | |
| | | | | GCLVKDYFPEPVTVSWNSGALTSG | |
| | | | | VHTFPAVLQSSGLYSLSSVVTVPSS | |
| | | | | SLGTQTYICNVNHKPSNTKVDKRV | |
| | | | | EPKSCDKTHTCPPCPAPEAAGGPSV | |
| | | | | FLFPPKPKDTLMISRTPEVTCVVVD | |
| | | | | VSHEDPEVKFNWYVDGVEVHNAK | |
| | | | | TKPREEQYNSTYRVVSVLTVLHQD | |
| | | | | WLNGKEYKCKVSNKALPAPIEKTIS | |
| | | | | KAKGQPREPQVYTLPPCREEMTKN | |
| | | | | QVSLWCLVKGFYPSDIAVEWESNG | |
| | | | | QPENNYKTTPPVLDSDGSFFLYSKL | |
| | | | | TVDKSRWQQGNVFSCSVMHEALH | |
| | | | | NHYTQKSLSLSPG | |
| | | IO- | aLILRB4 | EVQLVESGGGLVQPGGSLRLSCAA | 173 |
| | | 312 HC | VH-TCRβ- | SGFSLSSSYWISWVRQAPGKGLEW | |
| | | 2 | IgG1FcHole | IGSIDSGSVGITYYATWVKGRFTIS | |
| | | | (L234A, | RDNSKNTLYLQMNSLRAEDTAVY | |
| | | | L235A) | YCARHGDNWALDLWGQGTLVTV | |
| | | | | LE/DLKNVFPPEVAVFEPSEAEISHT | |
| | | | | QKATLVCLATGFYPDHVELSWWV | |
| | | | | NGKEVHSGVCTDPQPLKEQPALQD | |
| | | | | SRYALSSRLRVSATFWQNPRNHFR | |
| | | | | CQVQFYGLSENDEWTQDRAKPVT | |
| | | | | QIVSAEAWGRAS/DKTHTCPPCPAP | |
| | | | | EAAGGPSVFLFPPKPKDTLMISRTP | |
| | | | | EVTCVVVDVSHEDPEVKFNWYVD | |
| | | | | GVEVHNAKTKPREEQYNSTYRVVS | |
| | | | | VLTVLHQDWLNGKEYKCKVSNKA | |
| | | | | LPAPIEKTISKAKGQPREPQVCTLPP | |
| | | | | SREEMTKNQVSLSCAVKGFYPSDI | |
| | | | | AVEWESNGQPENNYKTTPPVLDSD | |
| | | | | GSFFLVSKLTVDKSRWQQGNVFSC | |
| | | | | SVMHEALHNHYTQKSLSLSPG | |
| | | IO- | aCD3VL-CL | DIVMTQSPDSLAVSLGERATINCKS | 174 |
| | | 312 LC1 | Kappa | SQSLLNSRTRKNYLAWYQQKPGQP | |
| | | | | PKLLIDIVMTQSPDSLAVSLGERATI | |
| | | | | NCKSSQSLLNSRTRKNYLAWYQQ | |
| | | | | KPGQPPKLLIYWASTRQSGVPDRF | |
| | | | | SGSGSGTDFTLTISSLQAEDVAVYY | |
| | | | | CTQSHTLRTFGGGTKVEIK/RTVA | |
| | | | | APSVFIFPPSDEQLKSGTASVVCLL | |
| | | | | NNFYPREAKVQWKVDNALQSGNS | |
| | | | | QESVTEQDSKDSTYSLSSTLTLSKA | |
| | | | | DYEKHKVYACEVTHQGLSSPVTKS | |
| | | | | FNRGEC | |
| | | IO- | aLILRB4 | DIQMTQSPSTLSASVGDRVTITCRA | 175 |
| | | 312 LC2 | VL-TCRα | SQSISSWLAWYQQKPGKAPKLLIY | |
| | | | | KASTLASGVPSRFSGSGSGTEFTLTI | |
| | | | | SSLQPDDFATYYCQHGYIRGDLDN | |
| | | | | VFGGGTKVEIK/PDIQNPDPAVYQL | |
| | | | | RDSKSSDKSVCLFTDFDSQTQVSQS | |
| | | | | KDSDVYITDKCVLDMRSMDFKSNS | |
| | | | | AVAWSQKSDFACANAFQNSIIPEDT | |
| | | | | FFPAPESS | |
| LIL | | ATG312 | aLILRB4 | EVQLVESGGGLVQPGGSLRLSCAA | 176 |
| RB4 | | -1 HC1 | VH-IgG1 | SGFSLSSSYWISWVRQAPGKGLEW | |
| xCD3 | | | Hole | IGSIDSGSVGITYYATWVKGRFTIS | |
| BsA | | | (L234A, | RDNSKNTLYLQMNSLRAEDTAVY | |
| b | | | L235A) | YCARHGDNWALDLWGQGTLVTV | |
| | | | | LE/ASTKGPSVFPLAPSSKSTSGGTA | |
| | | | | ALGCLVKDYFPEPVTVSWNSGALT | |
| | | | | SGVHTFPAVLQSSGLYSLSSVVTVP | |
| | | | | SSSLGTQTYICNVNHKPSNTKVDK | |
| | | | | KVEPKSCDKTHTCPPCPAPEAAGG | |
| | | | | PSVFLFPPKPKDTLMISRTPEVTCV | |
| | | | | VVDVSHEDPEVKFNWYVDGVEVH | |
| | | | | NAKTKPREEQYNSTYRVVSVLTVL | |
| | | | | HQDWLNGKEYKCKVSNKALPAPIE | |
| | | | | KTISKAKGQPREPQVYTLPPCREEM | |
| | | | | TKNQVSLWCLVKGFYPSDIAVEWE | |
| | | | | SNGQPENNYKTTPPVLDSDGSFFLY | |
| | | | | SKLTVDKSRWQQGNVFSCSVMHE | |
| | | | | ALHNHYTQKSLSLSPGK | |
| | AT | ATG312 | aLILRB4 | EVQLVESGGGLVQPGGSLRLSCAA | 177 |
| | G31 | -1 HC2 | VH-CH1- | SGFSLSSSYWISWVRQAPGKGLEW | |
| | 2-1 | | aCD3scFv | IGSIDSGSVGITYYATWVKGRFTIS | |
| | | | (C-C)-IgG1 | RDNSKNTLYLQMNSLRAEDTAVY | |
| | | | Knob | YCARHGDNWALDLWGQGTLVTV | |
| | | | (L234A, | LE/ASTKGPSVFPLAPSSKSTSGGTA | |
| | | | L235A) | ALGCLVKDYFPEPVTVSWNSGALT | |
| | | | | SGVHTFPAVLQSSGLYSLSSVVTVP | |
| | | | | SSSLGTQTYICNVNHKPSNTKVDK | |
| | | | | KVEPKSC/GGGGSGGGGS/QVQLVQ | |
| | | | | SGAEVKKPGSSVKVSCKASGFAFT | |
| | | | | DYYIHWVRQAPGQCLEWMGWISP | |
| | | | | GNVNTKYNENFKGRVTITADKSTS | |
| | | | | TAYMELSSLRSEDTAVYYCARDGY | |
| | | | | SLYYFDYWGQGTLVTVSS/GGGGS | |
| | | | | GGGGSGGGGSGGGGS/DIVMTQSP | |
| | | | | DSLAVSLGERATINCKSSQSLLNSR | |
| | | | | TRKNYLAWYQQKPGQPPKLLIYW | |
| | | | | ASTRQSGVPDRFSGSGSGTDFTLTI | |
| | | | | SSLQAEDVAVYYCTQSHTLRTFGC | |
| | | | | GTKVEIK/GGGGSGGGGS/DKTHTC | |
| | | | | PPCPAPEAAGGPSVFLFPPKPKDTL | |
| | | | | MISRTPEVTCVVVDVSHEDPEVKF | |
| | | | | NWYVDGVEVHNAKTKPREEQYNS | |
| | | | | TYRVVSVLTVLHQDWLNGKEYKC | |
| | | | | KVSNKALPAPIEKTISKAKGQPREP | |
| | | | | QVCTLPPSREEMTKNQVSLSCAVK | |
| | | | | GFYPSDIAVEWESNGQPENNYKTT | |
| | | | | PPVLDSDGSFFLVSKLTVDKSRWQ | |
| | | | | QGNVFSCSVMHEALHNHYTQKSLS | |
| | | | | LSPGK | |
| | | ATG312 | aLILRB4 | DIQMTQSPSTLSASVGDRVTITCRA | 178 |
| | | -1 LC | VL-CL | SQSISSWLAWYQQKPGKAPKLLIY | |
| | | | Kappa | KASTLASGVPSRFSGSGSGTEFTLTI | |
| | | | | SSLQPDDFATYYCQHGYIRGDLDN | |
| | | | | VFGGGTKVEIK/RTVAAPSVFIFPPS | |
| | | | | DEQLKSGTASVVCLLNNFYPREAK | |
| | | | | VQWKVDNALQSGNSQESVTEQDS | |
| | | | | KDSTYSLSSTLTLSKADYEKHKVY | |
| | | | | ACEVTHQGLSSPVTKSFNRGEC | |
| LIL | AT | ATG312 | aLILRB4 | EVQLVESGGGLVQPGGSLRLSCAA | 179 |
| RB4 | G31 | -2 HC1 | VH-IgG1 | SGFDFSRYWMSWVRQAPGKGLV | |
| xCD | | | Hole | WIGEINPESSAINYAPSLKDRFTISR | |
| 3 | | | (L234A, | DNAKNTLYLQMNSLRAEDTAVYY | |
| BsA | | | L235A) | CATSMTMDYWGQGTLVTVSS/AST | |
| b | | | | KGPSVFPLAPSSKSTSGGTAALGCL | |
| | | | | VKDYFPEPVTVSWNSGALTSGVHT | |
| | | | | FPAVLQSSGLYSLSSVVTVPSSSLG | |
| | | | | TQTYICNVNHKPSNTKVDKKVEPK | |
| | | | | SCDKTHTCPPCPAPEAAGGPSVFLF | |
| | | | | PPKPKDTLMISRTPEVTCVVVDVSH | |
| | | | | EDPEVKFNWYVDGVEVHNAKTKP | |
| | | | | REEQYNSTYRVVSVLTVLHQDWL | |
| | | | | NGKEYKCKVSNKALPAPIEKTISKA | |
| | | | | KGQPREPQVYTLPPCREEMTKNQV | |
| | | | | SLWCLVKGFYPSDIAVEWESNGQP | |
| | | | | ENNYKTTPPVLDSDGSFFLYSKLTV | |
| | | | | DKSRWQQGNVFSCSVMHEALHNH | |
| | | | | YTQKSLSLSPGK | |
| | 2-2 | ATG312 | aLILRB4 | EVQLVESGGGLVQPGGSLRLSCAA | 180 |
| | | -2 HC2 | VH-CH1- | SGFDFSRYWMSWVRQAPGKGLV | |
| | | | aCD3scFv | WIGEINPESSAINYAPSLKDRFTISR | |
| | | | (C-C)-IgG1 | DNAKNTLYLQMNSLRAEDTAVYY | |
| | | | Knob | CATSMTMDYWGQGTLVTVSS/AST | |
| | | | (L234A, | KGPSVFPLAPSSKSTSGGTAALGCL | |
| | | | L235A) | VKDYFPEPVTVSWNSGALTSGVHT | |
| | | | | FPAVLQSSGLYSLSSVVTVPSSSLG | |
| | | | | TQTYICNVNHKPSNTKVDKKVEPK | |
| | | | | SC/GGGGSGGGGS/QVQLVQSGAEV | |
| | | | | KKPGSSVKVSCKASGFAFTDYYIH | |
| | | | | WVRQAPGQCLEWMGWISPGNVN | |
| | | | | TKYNENFKGRVTITADKSTSTAYM | |
| | | | | ELSSLRSEDTAVYYCARDGYSLYY | |
| | | | | FDYWGQGTLVTVSS/GGGGSGGGG | |
| | | | | SGGGGSGGGGS/DIVMTQSPDSLAV | |
| | | | | SLGERATINCKSSQSLLNSRTRKN | |
| | | | | YLAWYQQKPGQPPKLLIYWASTR | |
| | | | | QSGVPDRFSGSGSGTDFTLTISSLQ | |
| | | | | AEDVAVYYCTQSHTLRTFGCGTK | |
| | | | | VEIK/GGGGSGGGGS/DKTHTCPPCP | |
| | | | | APEAAGGPSVFLFPPKPKDTLMISR | |
| | | | | TPEVTCVVVDVSHEDPEVKFNWY | |
| | | | | VDGVEVHNAKTKPREEQYNSTYR | |
| | | | | VVSVLTVLHQDWLNGKEYKCKVS | |
| | | | | NKALPAPIEKTISKAKGQPREPQVC | |
| | | | | TLPPSREEMTKNQVSLSCAVKGFY | |
| | | | | PSDIAVEWESNGQPENNYKTTPPVL | |
| | | | | DSDGSFFLVSKLTVDKSRWQQGNV | |
| | | | | FSCSVMHEALHNHYTQKSLSLSPG | |
| | | | | K | |
| | | ATG312 | aLILRB4 | DIQMTQSPSSLSASVGDRVTITCKA | 181 |
| | | -2 LC | VL-CL | SQDIKSYLSWYQQKPGKAPKTLIY | |
| | | | Kappa | YATSLAGGVPSRFSGSGSGTDYTL | |
| | | | | TISSLQPEDFATYYCLQHGESPYTF | |
| | | | | GGGTKLEIK/RTVAAPSVFIFPPSDE | |
| | | | | QLKSGTASVVCLLNNFYPREAKVQ | |
| | | | | WKVDNALQSGNSQESVTEQDSKD | |
| | | | | STYSLSSTLTLSKADYEKHKVYAC | |
| | | | | EVTHQGLSSPVTKSFNRGEC | |
| LIL | IO- | IO-312- | aLILRB4VH- | EVQLVESGGGLVQPGGSLRLSCAASG | 235 |
| RB4 | 312- | N55S_HC | TCRβ-aCD3 | FSLSSSYWISWVRQAPGKGLEWIGSID | |
| xCD | N55 | 1 | VH-IgG1 | SGSVGITYYATWVKGRFTISRDNSKNT | |
| 3 | S | | Knob(L234A, | LYLQMNSLRAEDTAVYYCARHGDNW | |
| BsA | | | L235A) | ALDLWGQGTLVTVLE/DLKNVFPPEV | |
| b | | | | AVFEPSEAEISHTQKATLVCLATGFYP | |
| | | | | DHVELSWWVNGKEVHSGVCTDPQPL | |
| | | | | KEQPALQDSRYALSSRLRVSATFWQN | |
| | | | | PRNHFRCQVQFYGLSENDEWTQDRAK | |
| | | | | PVTQIVSAEAWGR/GGGGSGGGGS/EV | |
| | | | | QLVQSGAEVKKPGSSVKVSCKASGYT | |
| | | | | FSSYVIHWVRQAPGQGLEWMGYINPY | |
| | | | | SDVIKYNEKFKGRVTLTSDKSTSTAY | |
| | | | | MELSSLRSEDTAVYYCARDSYYSYDG | |
| | | | | FAYWGQGTLVTVSS/ASTKGPSVFPLA | |
| | | | | PSSKSTSGGTAALGCLVKDYFPEPVTV | |
| | | | | SWNSGALTSGVHTFPAVLQSSGLYSLS | |
| | | | | SVVTVPSSSLGTQTYICNVNHKPSNTK | |
| | | | | VDKRVEPKSCDKTHTCPPCPAPEAAG | |
| | | | | GPSVFLFPPKPKDTLMISRTPEVTCVV | |
| | | | | VDVSHEDPEVKFNWYVDGVEVHNAK | |
| | | | | TKPREEQYNSTYRVVSVLTVLHQDWL | |
| | | | | NGKEYKCKVSNKALPAPIEKTISKAKG | |
| | | | | QPREPQVYTLPPCREEMTKNQVSLWC | |
| | | | | LVKGFYPSDIAVEWESNGQPENNYKT | |
| | | | | TPPVLDSDGSFFLYSKLTVDKSRWQQ | |
| | | | | GNVFSCSVMHEALHNHYTQKSLSLSP | |
| | | | | G | |
| | | IO-312- | aLILRB4VH- | EVQLVESGGGLVQPGGSLRLSCAASG | 236 |
| | | N55S HC | TCRβ-IgG1Fc | FSLSSSYWISWVRQAPGKGLEWIGSID | |
| | | 2 | Hole(L234A, | SGSVGITYYATWVKGRFTISRDNSKNT | |
| | | | L235A) | LYLQMNSLRAEDTAVYYCARHGDNW | |
| | | | | ALDLWGQGTLVTVLE/DLKNVFPPEV | |
| | | | | AVFEPSEAEISHTQKATLVCLATGFYP | |
| | | | | DHVELSWWVNGKEVHSGVCTDPQPL | |
| | | | | KEQPALQDSRYALSSRLRVSATFWQN | |
| | | | | PRNHFRCQVQFYGLSENDEWTQDRAK | |
| | | | | PVTQIVSAEAWGRAS/DKTHTCPPCPA | |
| | | | | PEAAGGPSVFLFPPKPKDTLMISRTPEV | |
| | | | | TCVVVDVSHEDPEVKFNWYVDGVEV | |
| | | | | HNAKTKPREEQYNSTYRVVSVLTVLH | |
| | | | | QDWLNGKEYKCKVSNKALPAPIEKTIS | |
| | | | | KAKGQPREPQVCTLPPSREEMTKNQV | |
| | | | | SLSCAVKGFYPSDIAVEWESNGQPENN | |
| | | | | YKTTPPVLDSDGSFFLVSKLTVDKSRW | |
| | | | | QQGNVFSCSVMHEALHNHYTQKSLSL | |
| | | | | SPG | |
| | | IO-312- | aCD3VL-CL | EIVLTQSPDFQSVTPKEKVTITCSASSS | 237 |
| | | N55S_LC | Kappa | VSYMHWYQQKPDQSPKRLIYDTSKLA | |
| | | 1 | | SGVPSRFSGSGSGTDFTLTINSLEAEDA | |
| | | | | ATYYCQQWSSNPPTFGGGTKVEIK/RT | |
| | | | | VAAPSVFIFPPSDEQLKSGTASVVCLL | |
| | | | | NNFYPREAKVQWKVDNALQSGNSQE | |
| | | | | SVTEQDSKDSTYSLSSTLTLSKADYEK | |
| | | | | HKVYACEVTHQGLSSPVTKSFNRGEC | |
| | | IO-312- | aLILRB4VL- | DIQMTQSPSTLSASVGDRVTITCRASQ | 238 |
| | | N55S_LC | TCRα | SISSWLAWYQQKPGKAPKLLIYKASTL | |
| | | 2 | | ASGVPSRFSGSGSGTEFTLTISSLQPDD | |
| | | | | FATYYCQHGYIRGDLDNVFGGGTKVE | |
| | | | | IK/PDIQNPDPAVYQLRDSKSSDKSVCL | |
| | | | | FTDFDSQTQVSQSKDSDVYITDKCVLD | |
| | | | | MRSMDFKSNSAVAWSQKSDFACANA | |
| | | | | FQNSIIPEDTFFPAPESS | |
| LIL | IO- | IO-312- | aLILRB4VH- | EVQLVESGGGLVQPGGSLRLSCAASG | 239 |
| RB4 | 312- | L2H3_H | TCRβ-aCD3 | FSLSSSYWISWVRQAPGKGLEWIGSID | |
| xCD | L2H | C1 | VH-IgG1 | SGSVGITYYATWVKGRFTISRDNSKNT | |
| 3 | 3 | | Knob(L234A, | LYLQMNSLRAEDTAVYYCARHGDNW | |
| BsA | | | L235A) | ALDLWGQGTLVTVLE/DLKNVFPPEV | |
| b | | | | AVFEPSEAEISHTQKATLVCLATGFYP | |
| | | | | DHVELSWWVNGKEVHSGVCTDPQPL | |
| | | | | KEQPALQDSRYALSSRLRVSATFWQN | |
| | | | | PRNHFRCQVQFYGLSENDEWTQDRAK | |
| | | | | PVTQIVSAEAWGR/GGGGSGGGGS/EV | |
| | | | | QLVQSGAEVKKPGSSVKVSCKASGYT | |
| | | | | FSSYVIHWVRQAPGQGLEWMGYINPY | |
| | | | | NDVIKYNEKFKGRVTLTSDKSTSTAY | |
| | | | | MELSSLRSEDTAVYYCARDSYYSYDG | |
| | | | | FAYWGQGTLVTVSS/ASTKGPSVFPLA | |
| | | | | PSSKSTSGGTAALGCLVKDYFPEPVTV | |
| | | | | SWNSGALTSGVHTFPAVLQSSGLYSLS | |
| | | | | SVVTVPSSSLGTQTYICNVNHKPSNTK | |
| | | | | VDKRVEPKSCDKTHTCPPCPAPEAAG | |
| | | | | GPSVFLFPPKPKDTLMISRTPEVTCVV | |
| | | | | VDVSHEDPEVKFNWYVDGVEVHNAK | |
| | | | | TKPREEQYNSTYRVVSVLTVLHQDWL | |
| | | | | NGKEYKCKVSNKALPAPIEKTISKAKG | |
| | | | | QPREPQVYTLPPCREEMTKNQVSLWC | |
| | | | | LVKGFYPSDIAVEWESNGQPENNYKT | |
| | | | | TPPVLDSDGSFFLYSKLTVDKSRWQQ | |
| | | | | GNVFSCSVMHEALHNHYTQKSLSLSP | |
| | | | | G | |
| | | IO-312- | aLILRB4VH- | EVQLVESGGGLVQPGGSLRLSCAASG | 240 |
| | | L2H3_H | TCRβ-IgG1Fc | FSLSSSYWISWVRQAPGKGLEWIGSID | |
| | | C2 | Hole(L234A, | SGSVGITYYATWVKGRFTISRDNSKNT | |
| | | | L235A) | LYLQMNSLRAEDTAVYYCARHGDNW | |
| | | | | ALDLWGQGTLVTVLE/DLKNVFPPEV | |
| | | | | AVFEPSEAEISHTQKATLVCLATGFYP | |
| | | | | DHVELSWWVNGKEVHSGVCTDPQPL | |
| | | | | KEQPALQDSRYALSSRLRVSATFWQN | |
| | | | | PRNHFRCQVQFYGLSENDEWTQDRAK | |
| | | | | PVTQIVSAEAWGRAS/DKTHTCPPCPA | |
| | | | | PEAAGGPSVFLFPPKPKDTLMISRTPEV | |
| | | | | TCVVVDVSHEDPEVKFNWYVDGVEV | |
| | | | | HNAKTKPREEQYNSTYRVVSVLTVLH | |
| | | | | QDWLNGKEYKCKVSNKALPAPIEKTIS | |
| | | | | KAKGQPREPQVCTLPPSREEMTKNQV | |
| | | | | SLSCAVKGFYPSDIAVEWESNGQPENN | |
| | | | | YKTTPPVLDSDGSFFLVSKLTVDKSRW | |
| | | | | QQGNVFSCSVMHEALHNHYTQKSLSL | |
| | | | | SPG | |
| | | IO-312- | aCD3VL-CL | EIVLTQSPDFQSVTPKEKVTITCSASSS | 241 |
| | | L2H3_LC | Kappa | VSYMHWYQQKPDQSPKRLIYDTSKLA | |
| | | 1 | | SGVPSRFSGSGSGTDFTLTINSLEAEDA | |
| | | | | ATYYCQQWSSNPPTFGGGTKVEIK/RT | |
| | | | | VAAPSVFIFPPSDEQLKSGTASVVCLL | |
| | | | | NNFYPREAKVQWKVDNALQSGNSQE | |
| | | | | SVTEQDSKDSTYSLSSTLTLSKADYEK | |
| | | | | HKVYACEVTHQGLSSPVTKSFNRGEC | |
| | | IO-312- | aLILRB4VL- | DIQMTQSPSTLSASVGDRVTITCRASQ | 242 |
| | | L2H3_LC | TCRα | SISSWLAWYQQKPGKAPKLLIYKASTL | |
| | | 2 | | ASGVPSRFSGSGSGTEFTLTISSLQPDD | |
| | | | | FATYYCQHGYIRGDLDNVFGGGTKVE | |
| | | | | IK/PDIQNPDPAVYQLRDSKSSDKSVCL | |
| | | | | FTDFDSQTQVSQSKDSDVYITDKCVLD | |
| | | | | MRSMDFKSNSAVAWSQKSDFACANA | |
| | | | | FQNSIIPEDTFFPAPESS | |

The protocol for FACS assay is described as follows:
1. Isolated T cells from fresh human PBMC and resuspended in complete RPMI1640 media.
2. Seeded 5×10⁴, 100 μL/well T cells to 96-well cell culture plate.
3. Seeded 1×10⁴, 50 μL/well tumor cells to the plate.
4. Added 50 μL/well 4× final conc. increasing concentrations of antibodies beginning with max conc. 10 nM.
5. After 24 hrs and 72 hrs incubation, centrifuged at 400 g for 5 min and collected cells followed by CD69 (24 hrs) and CD25 (72 hrs) analysis gated from CD8+ T cells by FACS.

### 8.2 Results

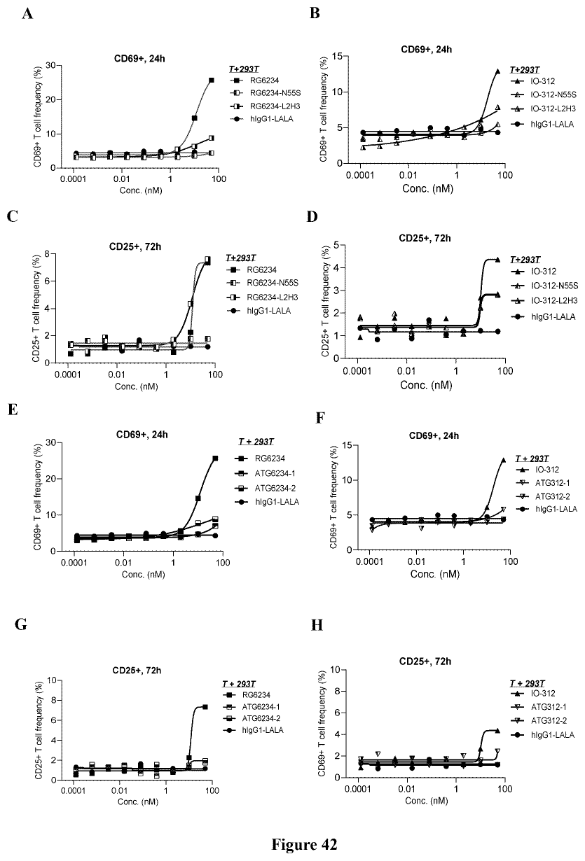

In the naïve T cell activation experiment (without TAA), after anti-CD3 sequence of RG6234 or IO-312 was replaced by anti-CD3 sequences 40E9-N55S and 40E9-L2H3 of the instant invention, T cell activation markers CD69 and CD25 of RG6234-N55S, RG6234-L2H3, IO-312-N55S and IO-312-L2H3 (Table 43) were dramatically decreased whereas the parental RG6234 and IO-312 induced marked T cell activation with upregulation of CD69 and CD25 (FIG. 42A-D). These suggested our anti-CD3 sequences alone provided masking effect to RG6234 and IO-312 before TAA crosslinking, enabling TAA-dependent conditional activity.

When anti-CD3 arm and anti-GPRC5D arm or anti-LILRB4 arm from RG6234 and IO-312, respectively was introduced into our Antengager format, T cell activation markers CD69 and CD25 of ATG6234-1, ATG6234-2, ATG312-1 and ATG-312-2 (Table 43) were also dramatically attenuated whereas the parental RG6234 and IO-312 induced marked T cell activation with upregulation of CD69 and CD25 (FIG. 42E-H). These indicated our Antengager format alone provided masking effect before TAA crosslinking, enabling TAA-dependent conditional activity.

### Example 9, CD3 Bispecific Antibody Characterization: T Cell Activation and T Cell-dependent Cellular Cytotoxicity in the Presence of Positive Target Cells

### 9.1 Methods

5×10⁴/well CFSE labeled target cells as target cells were seeded to 96 wells Flat-bottom sterile plate, then 2×10⁵ freshly isolated T cells from human PBMCs were added as effector cell. After that, human T cells, CFSE labeled target cells, and increasing concentrations of antibodies (human IgG1-LALA as a negative control) were combined in 200 μL total in RPMI-1640 medium in U-shaped 96-well plate and incubated for 24 hours at 37 degree, 5% CO₂ for 24 hours. Finally, cells were washed and resuspended in 200 μL of staining buffer. Cells were acquired by FACS and percentages of CFSE labeled target cells were measured.

Percent of cell cytotoxicity=100−([AT/NAT]×100)

(AT: antibody treated group, NAT: no antibody treated group)

### 9.2 Results

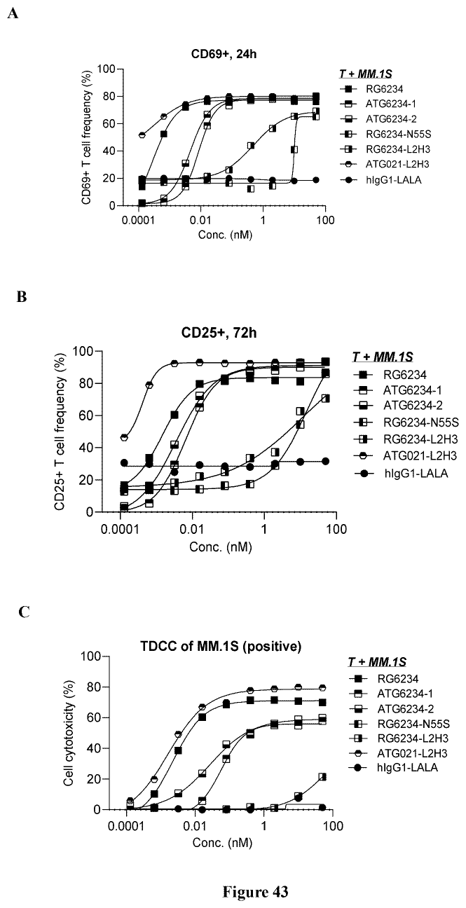

In the T cell activation (FIG. 43A-B) and T cell dependent cytotoxicity (TDCC) (FIG. 43C) experiment, in the presence of TAA⁺ cells, ATG-6234-1, ATG-6234-2, RG6234-N55S and RG6234-L2H3 induced lower CD69 and CD25 upregulation, and lower TDCC effect compared to RG6234 and Bis-40E9-L2H3 in the presence of GPRC5D+MM.1s cells (FIG. 43A-C). These results indicated that 1) anti-CD3 arm with linear epitope reduced TAA-dependent activation and cytotoxicity in Antengager format compared to Bis-40E9-L2H3 with anti-CD3 sequences 40E9-N55S and 40E9-L2H3, which target conformational epitope on CD3 complex; 2) Introducing anti-CD3 with conformational epitope into Fab3 configuration also reduced the T cell dependent activity (FIG. 42A-D).

In T cell activation and TDCC with the presence of MM.1S cells, Bis-40E9-L2H3 triggered the strongest activity among RG6234, ATG-6234-1, ATG-6234-2, RG6234-N55S, RG6234-L2H3, and Bis-40E9-L2H3 (FIG. 43A-C). Taken together, these suggested that the optimal T cell activation and killing only when CD3 binding arm with conformational epitope and Antengager format are simultaneously present. So that only anti-CD3 that binds to conformational epitope is compatible with Antengager format. Therefore, the presence of our Antengager format, combined with our anti-CD3 sequences, contributed to the most potent T cell activation and killing effect.

### Example 10. CD19×CD3 and CD20×CD3 Bispecific Antibody Generation and Characterization

### 10.1 CD19×CD3 and CD20×CD3 Bispecific Antibody Generation

Bispecific antibody targeting CD19 and CD3 or CD20 and CD3 were generated to evaluate the antibody characterization, including binding affinity, T cell activation with Jurkat NFAT luciferase reporter cells, T cell dependent cytotoxicity and PBMC cytotoxicity. In particular, each of the bispecific antibodies were generated in a Fab-scFv-Fab format, comprising two anti-CD19 or two anti-CD20 Fab domains and one higher affinity anti-CD3 single chain fragment variable (scFv) binding domain. Regarding the generated CD19×CD3 bispecific antibody, each of the anti-CD19 Fab domains comprises a CD19 binding moiety comprising a VH region and a VL region from the clone C19F or C19B, and the anti-CD3 scFv domain comprises the VH and VL regions from the clone 40E9-L2H3-Y101F.H, 40E9-L2H3-N55S.H or 40E9-L2H3-D105R.H. Regarding the generated CD20×CD3 bispecific antibody, each of the anti-CD20 Fab domains comprises a CD20 binding moiety comprising a VH region and a VL region from the clone C200 or C20R, and the anti-CD3 scFv domain comprises the VH and VL regions from the clone 40E9-L2H3-Y101F.H, 40E9-L2H3-N55S.H or 40E9-L2H3-D105R.H. In the generated Fab-scFv-Fab bispecific antibody, the VH and VL regions of the anti-CD3 scFv domain were linked by a (GGGGS)₄ linker, and the VL region of the anti-CD3 scFv domain was linked to the N-terminal of the CH2 domain of the generated antibody by a (GGGGS)₂ linker, the VH region of the anti-CD3 scFv domain was linked to CH1 region of one of the anti-CD19 or anti-CD20 Fab domains by a (GGGGS)₂ linker. LALA mutations (L234A, L235A, according to EU numbering) were introduced to each bispecific antibody to abolish the Fc receptor binding capability. The resulting 2+1 bispecific antibodies are referred to herein as ATG201-Y101F-19F, ATG201-Y101F-19B, ATG201-Y101F-200, ATG201-Y101F-20R, ATG201-N55S-19F, ATG201-N55S-19B, ATG201-N55S-200, ATG201-N55S-20R and ATG201-D105R-19B. The SEQ ID NOs and amino acid sequences of VL and VH regions of the Fab domain targeting CD19 or CD20 (i.e., C19F, C19B, C200, C20R) in each of the generated humanized 2+1 bispecific antibodies are shown in Table 48 below, and CDR sequences identified by the convention of Kabat are bold and underlined in each sequence listed in Table 48 below. The amino acid sequence of each chain of each generated 2+1 bispecific antibody is shown in Table 56 below.

TABLE 48
Amino Acid Sequences of VH and VL Regions of the Fab Domain
Targeting CD19 or CD20

| Sequence | | SEQ ID | |
|---|---|---|---|
| Description | | NO | Amino Acid Sequence |
| C19F | VH | 257 | EVKLQESGPGLVAPSQSLSVTCTVSGVSLP |
| | | | DYGVSWIRQPPRKGLEWLGVIWGSETTY |
| | | | YNSALKSRLTIIKDNSKSQVFLKMNSLQT |
| | | | DDTAIYYCAKHYYYGGSYAMDYWGQGT |
| | | | SVTVSS |
| | VL | 258 | DIQMTQTTSSLSASLGDRVTISCRASQDIS |
| | | | KYLNWYQQKPDGTVKLLIYHTSRLHSGV |
| | | | PSRFSGSGSGTDYSLTISNLEQEDIATYFCQ |
| | | | QGNTLPYTFGGGTKLEIT |
| C19B | VH | 270 | QVQLQQSGAELVRPGSSVKISCKASGYAFS |
| | | | SYWMNWVKQRPGQGLEWIGQIWPGDG |
| | | | DTNYNGKFKGKATLTADESSSTAYMQLSS |
| | | | LASEDSAVYFCARRETTTVGRYYYAMDY |
| | | | WGQGTTVTVSS |
| | VL | 271 | DIQLTQSPASLAVSLGQRATISCKASQSVD |
| | | | YDGDSYLNWYQQIPGQPPKLLIYDASNLV |
| | | | SGIPPRFSGSGSGTDFTLNIHPVEKVDAATY |
| | | | HCQQSTEDPWTFGGGTKLEIK |
| C200 | VH | 282 | EVQLVESGGGLVQPGRSLRLSCAASGFTFN |
| | | | DYAMHWVRQAPGKGLEWVSTISWNSGSI |
| | | | GYADSVKGRFTISRDNAKKSLYLQMNSLR |
| | | | AEDTALYYCAKDIQYGNYYYGMDVWGQ |
| | | | GTTVTVSS |
| | VL | 283 | EIVLTQSPATLSLSPGERATLSCRASQSVSS |
| | | | YLAWYQQKPGQAPRLLIYDASNRATGIPA |
| | | | RFSGSGSGTDFTLTISSLEPEDFAVYYCQQ |
| | | | RSNWPITFGQGTRLEIK |
| C20R | VH | 294 | QVQLQQPGAELVKPGASVKMSCKASGYT |
| | | | FTSYNMHWVKQTPGRGLEWIGAIYPGNG |
| | | | DTSYNQKFKGKATLTADKSSSTAYMQLSS |
| | | | LTSEDSAVYYCARSTYYGGDWYFNVWG |
| | | | AGTTVTVSA |
| | VL | 295 | QIVLSQSPAILSASPGEKVTMTCRASSSVSY |
| | | | IHWFQQKPGSSPKPWIYATSNLASGVPVR |
| | | | FSGSGSGTSYSLTISRVEAEDAATYYCQQ |
| | | | WTSNPPTFGGGTKLEIK |

TABLE 56
Sequence information of each generated 2 + 1 bispecific antibodies

| SEQ | | | |
|---|---|---|---|
| ID | Antibody | | |
| No | Code | Name | Amino Acid Sequence |
| 259 | ATG201- | heavy | EVKLQESGPGLVAPSQSLSVTCTVSGVSLPDYGVSWI |
| | Y101F- | chain | RQPPRKGLEWLGVIWGSETTYYNSALKSRLTIIKDNS |
| | 19F | 1 | KSQVFLKMNSLQTDDTAIYYCAKHYYYGGSYAMDY |
| | | | WGQGTSVTVSSASTKGPSVFPLAPSSKSTSGGTAALG |
| | | | CLVKDYFPEPVTVSWNSGALTSGVHTFPAVLQSSGL |
| | | | YSLSSVVTVPSSSLGTQTYICNVNHKPSNTKVDKKVE |
| | | | PKSCDKTHTCPPCPAPEAAGGPSVFLFPPKPKDTLMIS |
| | | | RTPEVTCVVVDVSHEDPEVKFNWYVDGVEVHNAKT |
| | | | KPREEQYNSTYRVVSVLTVLHQDWLNGKEYKCKVS |
| | | | NKALPAPIEKTISKAKGQPREPQVYTLPPCREEMTKN |
| | | | QVSLWCLVKGFYPSDIAVEWESNGQPENNYKTTPPV |
| | | | LDSDGSFFLYSKLTVDKSRWQQGNVFSCSVMHEALH |
| | | | NHYTQKSLSLSPGK |
| 260 | | heavy | EVKLQESGPGLVAPSQSLSVTCTVSGVSLPDYGVSWI |
| | | chain | RQPPRKGLEWLGVIWGSETTYYNSALKSRLTIIKDNS |
| | | 2 | KSQVFLKMNSLQTDDTAIYYCAKHYYYGGSYAMDY |
| | | | WGQGTSVTVSSASTKGPSVFPLAPSSKSTSGGTAALG |
| | | | CLVKDYFPEPVTVSWNSGALTSGVHTFPAVLQSSGL |
| | | | YSLSSVVTVPSSSLGTQTYICNVNHKPSNTKVDKKVE |
| | | | PKSCGGGGSGGGGSEVQLVQSGAEVKKPGSSVKVSC |
| | | | KASGYTFSSYVIHWVRQAPGQCLEWMGYINPYNDVI |
| | | | KYNEKFKGRVTLTSDKSTSTAYMELSSLRSEDTAVY |
| | | | YCARDSFYSYDGFAYWGQGTLVTVSSGGGGSGGGG |
| | | | SGGGGSGGGGSEIVLTQSPDFQSVTPKEKVTITCSASS |
| | | | SVSYMHWYQQKPDQSPKRLIYDTSKLASGVPSRFSG |
| | | | SGSGTDFTLTINSLEAEDAATYYCQQWSSNPPTFGCG |
| | | | TKVEIKGGGGSGGGGSDKTHTCPPCPAPEAAGGPSVF |
| | | | LFPPKPKDTLMISRTPEVTCVVVDVSHEDPEVKFNW |
| | | | YVDGVEVHNAKTKPREEQYNSTYRVVSVLTVLHQD |
| | | | WLNGKEYKCKVSNKALPAPIEKTISKAKGQPREPQV |
| | | | CTLPPSREEMTKNQVSLSCAVKGFYPSDIAVEWESN |
| | | | GQPENNYKTTPPVLDSDGSFFLVSKLTVDKSRWQQG |
| | | | NVFSCSVMHEALHNHYTQKSLSLSPGK |
| 261 | | light | DIQMTQTTSSLSASLGDRVTISCRASQDISKYLNWYQ |
| | | chain | QKPDGTVKLLIYHTSRLHSGVPSRFSGSGSGTDYSLTI |
| | | | SNLEQEDIATYFCQQGNTLPYTFGGGTKLEITRTVAA |
| | | | PSVFIFPPSDEQLKSGTASVVCLLNNFYPREAKVQWK |
| | | | VDNALQSGNSQESVTEQDSKDSTYSLSSTLTLSKADY |
| | | | EKHKVYACEVTHQGLSSPVTKSFNRGEC |
| 259 | ATG201- | heavy | EVKLQESGPGLVAPSQSLSVTCTVSGVSLPDYGVSWI |
| | N55S- | chain | RQPPRKGLEWLGVIWGSETTYYNSALKSRLTIIKDNS |
| | 19F | 1 | KSQVFLKMNSLQTDDTAIYYCAKHYYYGGSYAMDY |
| | | | WGQGTSVTVSSASTKGPSVFPLAPSSKSTSGGTAALG |
| | | | CLVKDYFPEPVTVSWNSGALTSGVHTFPAVLQSSGL |
| | | | YSLSSVVTVPSSSLGTQTYICNVNHKPSNTKVDKKVE |
| | | | PKSCDKTHTCPPCPAPEAAGGPSVFLFPPKPKDTLMIS |
| | | | RTPEVTCVVVDVSHEDPEVKFNWYVDGVEVHNAKT |
| | | | KPREEQYNSTYRVVSVLTVLHQDWLNGKEYKCKVS |
| | | | NKALPAPIEKTISKAKGQPREPQVYTLPPCREEMTKN |
| | | | QVSLWCLVKGFYPSDIAVEWESNGQPENNYKTTPPV |
| | | | LDSDGSFFLYSKLTVDKSRWQQGNVFSCSVMHEALH |
| | | | NHYTQKSLSLSPGK |
| 263 | | heavy | EVKLQESGPGLVAPSQSLSVTCTVSGVSLPDYGVSWI |
| | | chain | RQPPRKGLEWLGVIWGSETTYYNSALKSRLTIIKDNS |
| | | 2 | KSQVFLKMNSLQTDDTAIYYCAKHYYYGGSYAMDY |
| | | | WGQGTSVTVSSASTKGPSVFPLAPSSKSTSGGTAALG |
| | | | CLVKDYFPEPVTVSWNSGALTSGVHTFPAVLQSSGL |
| | | | YSLSSVVTVPSSSLGTQTYICNVNHKPSNTKVDKKVE |
| | | | PKSCGGGGSGGGGSEVQLVQSGAEVKKPGSSVKVSC |
| | | | KASGYTFSSYVIHWVRQAPGQCLEWMGYINPYSDVI |
| | | | KYNEKFKGRVTLTSDKSTSTAYMELSSLRSEDTAVY |
| | | | YCARDSYYSYDGFAYWGQGTLVTVSSGGGGSGGGG |
| | | | SGGGGSGGGGSEIVLTQSPDFQSVTPKEKVTITCSASS |
| | | | SVSYMHWYQQKPDQSPKRLIYDTSKLASGVPSRFSG |
| | | | SGSGTDFTLTINSLEAEDAATYYCQQWSSNPPTFGCG |
| | | | TKVEIKGGGGSGGGGSDKTHTCPPCPAPEAAGGPSVF |
| | | | LFPPKPKDTLMISRTPEVTCVVVDVSHEDPEVKFNW |
| | | | YVDGVEVHNAKTKPREEQYNSTYRVVSVLTVLHQD |
| | | | WLNGKEYKCKVSNKALPAPIEKTISKAKGQPREPQV |
| | | | CTLPPSREEMTKNQVSLSCAVKGFYPSDIAVEWESN |
| | | | GQPENNYKTTPPVLDSDGSFFLVSKLTVDKSRWQQG |
| | | | NVFSCSVMHEALHNHYTQKSLSLSPGK |
| 261 | | light | DIQMTQTTSSLSASLGDRVTISCRASQDISKYLNWYQ |
| | | chain | QKPDGTVKLLIYHTSRLHSGVPSRFSGSGSGTDYSLTI |
| | | | SNLEQEDIATYFCQQGNTLPYTFGGGTKLEITRTVAA |
| | | | PSVFIFPPSDEQLKSGTASVVCLLNNFYPREAKVQWK |
| | | | VDNALQSGNSQESVTEQDSKDSTYSLSSTLTLSKADY |
| | | | EKHKVYACEVTHQGLSSPVTKSFNRGEC |
| 272 | ATG201- | heavy | QVQLQQSGAELVRPGSSVKISCKASGYAFSSYWMN |
| | Y101F- | chain | WVKQRPGQGLEWIGQIWPGDGDTNYNGKFKGKATL |
| | 19B | 1 | TADESSSTAYMQLSSLASEDSAVYFCARRETTTVGR |
| | | | YYYAMDYWGQGTTVTVSSASTKGPSVFPLAPSSKST |
| | | | SGGTAALGCLVKDYFPEPVTVSWNSGALTSGVHTFP |
| | | | AVLQSSGLYSLSSVVTVPSSSLGTQTYICNVNHKPSN |
| | | | TKVDKKVEPKSCDKTHTCPPCPAPEAAGGPSVFLFPP |
| | | | KPKDTLMISRTPEVTCVVVDVSHEDPEVKFNWYVDG |
| | | | VEVHNAKTKPREEQYNSTYRVVSVLTVLHQDWLNG |
| | | | KEYKCKVSNKALPAPIEKTISKAKGQPREPQVYTLPP |
| | | | CREEMTKNQVSLWCLVKGFYPSDIAVEWESNGQPE |
| | | | NNYKTTPPVLDSDGSFFLYSKLTVDKSRWQQGNVFS |
| | | | CSVMHEALHNHYTQKSLSLSPGK |
| 273 | | heavy | QVQLQQSGAELVRPGSSVKISCKASGYAFSSYWMN |
| | | chain | WVKQRPGQGLEWIGQIWPGDGDTNYNGKFKGKATL |
| | | 2 | TADESSSTAYMQLSSLASEDSAVYFCARRETTTVGR |
| | | | YYYAMDYWGQGTTVTVSSASTKGPSVFPLAPSSKST |
| | | | SGGTAALGCLVKDYFPEPVTVSWNSGALTSGVHTFP |
| | | | AVLQSSGLYSLSSVVTVPSSSLGTQTYICNVNHKPSN |
| | | | TKVDKKVEPKSCGGGGSGGGGSEVQLVQSGAEVKK |
| | | | PGSSVKVSCKASGYTFSSYVIHWVRQAPGQCLEWM |
| | | | GYINPYNDVIKYNEKFKGRVTLTSDKSTSTAYMELSS |
| | | | LRSEDTAVYYCARDSFYSYDGFAYWGQGTLVTVSS |
| | | | GGGGSGGGGSGGGGSGGGGSEIVLTQSPDFQSVTPKE |
| | | | KVTITCSASSSVSYMHWYQQKPDQSPKRLIYDTSKL |
| | | | ASGVPSRFSGSGSGTDFTLTINSLEAEDAATYYCQQW |
| | | | SSNPPTFGCGTKVEIKGGGGSGGGGSDKTHTCPPCPA |
| | | | PEAAGGPSVFLFPPKPKDTLMISRTPEVTCVVVDVSH |
| | | | EDPEVKFNWYVDGVEVHNAKTKPREEQYNSTYRVV |
| | | | SVLTVLHQDWLNGKEYKCKVSNKALPAPIEKTISKA |
| | | | KGQPREPQVCTLPPSREEMTKNQVSLSCAVKGFYPS |
| | | | DIAVEWESNGQPENNYKTTPPVLDSDGSFFLVSKLTV |
| | | | DKSRWQQGNVFSCSVMHEALHNHYTQKSLSLSPGK |
| 274 | | light | DIQLTQSPASLAVSLGQRATISCKASQSVDYDGDSYL |
| | | chain | NWYQQIPGQPPKLLIYDASNLVSGIPPRFSGSGSGTDF |
| | | | TLNIHPVEKVDAATYHCQQSTEDPWTFGGGTKLEIK |
| | | | RTVAAPSVFIFPPSDEQLKSGTASVVCLLNNFYPREA |
| | | | KVQWKVDNALQSGNSQESVTEQDSKDSTYSLSSTLT |
| | | | LSKADYEKHKVYACEVTHQGLSSPVTKSFNRGEC |
| 272 | ATG201- | heavy | QVQLQQSGAELVRPGSSVKISCKASGYAFSSYWMN |
| | N55S- | chain | WVKQRPGQGLEWIGQIWPGDGDTNYNGKFKGKATL |
| | 19B | 1 | TADESSSTAYMQLSSLASEDSAVYFCARRETTTVGR |
| | | | YYYAMDYWGQGTTVTVSSASTKGPSVFPLAPSSKST |
| | | | SGGTAALGCLVKDYFPEPVTVSWNSGALTSGVHTFP |
| | | | AVLQSSGLYSLSSVVTVPSSSLGTQTYICNVNHKPSN |
| | | | TKVDKKVEPKSCDKTHTCPPCPAPEAAGGPSVFLFPP |
| | | | KPKDTLMISRTPEVTCVVVDVSHEDPEVKFNWYVDG |
| | | | VEVHNAKTKPREEQYNSTYRVVSVLTVLHQDWLNG |
| | | | KEYKCKVSNKALPAPIEKTISKAKGQPREPQVYTLPP |
| | | | CREEMTKNQVSLWCLVKGFYPSDIAVEWESNGQPE |
| | | | NNYKTTPPVLDSDGSFFLYSKLTVDKSRWQQGNVFS |
| | | | CSVMHEALHNHYTQKSLSLSPGK |
| 275 | | heavy | QVQLQQSGAELVRPGSSVKISCKASGYAFSSYWMN |
| | | chain | WVKQRPGQGLEWIGQIWPGDGDTNYNGKFKGKATL |
| | | 2 | TADESSSTAYMQLSSLASEDSAVYFCARRETTTVGR |
| | | | YYYAMDYWGQGTTVTVSSASTKGPSVFPLAPSSKST |
| | | | SGGTAALGCLVKDYFPEPVTVSWNSGALTSGVHTFP |
| | | | AVLQSSGLYSLSSVVTVPSSSLGTQTYICNVNHKPSN |
| | | | TKVDKKVEPKSCGGGGSGGGGSEVQLVQSGAEVKK |
| | | | PGSSVKVSCKASGYTFSSYVIHWVRQAPGQCLEWM |
| | | | GYINPYSDVIKYNEKFKGRVTLTSDKSTSTAYMELSS |
| | | | LRSEDTAVYYCARDSYYSYDGFAYWGQGTLVTVSS |
| | | | GGGGSGGGGSGGGGSGGGGSEIVLTQSPDFQSVTPKE |
| | | | KVTITCSASSSVSYMHWYQQKPDQSPKRLIYDTSKL |
| | | | ASGVPSRFSGSGSGTDFTLTINSLEAEDAATYYCQQW |
| | | | SSNPPTFGCGTKVEIKGGGGSGGGGSDKTHTCPPCPA |
| | | | PEAAGGPSVFLFPPKPKDTLMISRTPEVTCVVVDVSH |
| | | | EDPEVKFNWYVDGVEVHNAKTKPREEQYNSTYRVV |
| | | | SVLTVLHQDWLNGKEYKCKVSNKALPAPIEKTISKA |
| | | | KGQPREPQVCTLPPSREEMTKNQVSLSCAVKGFYPS |
| | | | DIAVEWESNGQPENNYKTTPPVLDSDGSFFLVSKLTV |
| | | | DKSRWQQGNVFSCSVMHEALHNHYTQKSLSLSPGK |
| 274 | | light | DIQLTQSPASLAVSLGQRATISCKASQSVDYDGDSYL |
| | | chain | NWYQQIPGQPPKLLIYDASNLVSGIPPRFSGSGSGTDF |
| | | | TLNIHPVEKVDAATYHCQQSTEDPWTFGGGTKLEIK |
| | | | RTVAAPSVFIFPPSDEQLKSGTASVVCLLNNFYPREA |
| | | | KVQWKVDNALQSGNSQESVTEQDSKDSTYSLSSTLT |
| | | | LSKADYEKHKVYACEVTHQGLSSPVTKSFNRGEC |
| 284 | ATG201- | heavy | EVQLVESGGGLVQPGRSLRLSCAASGFTFNDYAMH |
| | Y101F- | chain | WVRQAPGKGLEWVSTISWNSGSIGYADSVKGRFTIS |
| | 200 | 1 | RDNAKKSLYLQMNSLRAEDTALYYCAKDIQYGNYY |
| | | | YGMDVWGQGTTVTVSSASTKGPSVFPLAPSSKSTSG |
| | | | GTAALGCLVKDYFPEPVTVSWNSGALTSGVHTFPAV |
| | | | LQSSGLYSLSSVVTVPSSSLGTQTYICNVNHKPSNTK |
| | | | VDKKVEPKSCDKTHTCPPCPAPEAAGGPSVFLFPPKP |
| | | | KDTLMISRTPEVTCVVVDVSHEDPEVKFNWYVDGV |
| | | | EVHNAKTKPREEQYNSTYRVVSVLTVLHQDWLNGK |
| | | | EYKCKVSNKALPAPIEKTISKAKGQPREPQVYTLPPC |
| | | | REEMTKNQVSLWCLVKGFYPSDIAVEWESNGQPEN |
| | | | NYKTTPPVLDSDGSFFLYSKLTVDKSRWQQGNVFSC |
| | | | SVMHEALHNHYTQKSLSLSPGK |
| 285 | | heavy | EVQLVESGGGLVQPGRSLRLSCAASGFTFNDYAMH |
| | | chain | WVRQAPGKGLEWVSTISWNSGSIGYADSVKGRFTIS |
| | | 2 | RDNAKKSLYLQMNSLRAEDTALYYCAKDIQYGNYY |
| | | | YGMDVWGQGTTVTVSSASTKGPSVFPLAPSSKSTSG |
| | | | GTAALGCLVKDYFPEPVTVSWNSGALTSGVHTFPAV |
| | | | LQSSGLYSLSSVVTVPSSSLGTQTYICNVNHKPSNTK |
| | | | VDKKVEPKSCGGGGSGGGGSEVQLVQSGAEVKKPG |
| | | | SSVKVSCKASGYTFSSYVIHWVRQAPGQCLEWMGYI |
| | | | NPYNDVIKYNEKFKGRVTLTSDKSTSTAYMELSSLRS |
| | | | EDTAVYYCARDSFYSYDGFAYWGQGTLVTVSSGGG |
| | | | GSGGGGSGGGGSGGGGSEIVLTQSPDFQSVTPKEKVT |
| | | | ITCSASSSVSYMHWYQQKPDQSPKRLIYDTSKLASGV |
| | | | PSRFSGSGSGTDFTLTINSLEAEDAATYYCQQWSSNP |
| | | | PTFGCGTKVEIKGGGGSGGGGSDKTHTCPPCPAPEAA |
| | | | GGPSVFLFPPKPKDTLMISRTPEVTCVVVDVSHEDPE |
| | | | VKFNWYVDGVEVHNAKTKPREEQYNSTYRVVSVLT |
| | | | VLHQDWLNGKEYKCKVSNKALPAPIEKTISKAKGQP |
| | | | REPQVCTLPPSREEMTKNQVSLSCAVKGFYPSDIAVE |
| | | | WESNGQPENNYKTTPPVLDSDGSFFLVSKLTVDKSR |
| | | | WQQGNVFSCSVMHEALHNHYTQKSLSLSPGK |
| 286 | | light | EIVLTQSPATLSLSPGERATLSCRASQSVSSYLAWYQ |
| | | chain | QKPGQAPRLLIYDASNRATGIPARFSGSGSGTDFTLTI |
| | | | SSLEPEDFAVYYCQQRSNWPITFGQGTRLEIKRTVAA |
| | | | PSVFIFPPSDEQLKSGTASVVCLLNNFYPREAKVQWK |
| | | | VDNALQSGNSQESVTEQDSKDSTYSLSSTLTLSKADY |
| | | | EKHKVYACEVTHQGLSSPVTKSFNRGEC |
| 284 | ATG201- | heavy | EVQLVESGGGLVQPGRSLRLSCAASGFTFNDYAMH |
| | N55S- | chain | WVRQAPGKGLEWVSTISWNSGSIGYADSVKGRFTIS |
| | 200 | 1 | RDNAKKSLYLQMNSLRAEDTALYYCAKDIQYGNYY |
| | | | YGMDVWGQGTTVTVSSASTKGPSVFPLAPSSKSTSG |
| | | | GTAALGCLVKDYFPEPVTVSWNSGALTSGVHTFPAV |
| | | | LQSSGLYSLSSVVTVPSSSLGTQTYICNVNHKPSNTK |
| | | | VDKKVEPKSCDKTHTCPPCPAPEAAGGPSVFLFPPKP |
| | | | KDTLMISRTPEVTCVVVDVSHEDPEVKFNWYVDGV |
| | | | EVHNAKTKPREEQYNSTYRVVSVLTVLHQDWLNGK |
| | | | EYKCKVSNKALPAPIEKTISKAKGQPREPQVYTLPPC |
| | | | REEMTKNQVSLWCLVKGFYPSDIAVEWESNGQPEN |
| | | | NYKTTPPVLDSDGSFFLYSKLTVDKSRWQQGNVFSC |
| | | | SVMHEALHNHYTQKSLSLSPGK |
| 287 | | heavy | EVQLVESGGGLVQPGRSLRLSCAASGFTFNDYAMH |
| | | chain | WVRQAPGKGLEWVSTISWNSGSIGYADSVKGRFTIS |
| | | 2 | RDNAKKSLYLQMNSLRAEDTALYYCAKDIQYGNYY |
| | | | YGMDVWGQGTTVTVSSASTKGPSVFPLAPSSKSTSG |
| | | | GTAALGCLVKDYFPEPVTVSWNSGALTSGVHTFPAV |
| | | | LQSSGLYSLSSVVTVPSSSLGTQTYICNVNHKPSNTK |
| | | | VDKKVEPKSCGGGGSGGGGSEVQLVQSGAEVKKPG |
| | | | SSVKVSCKASGYTFSSYVIHWVRQAPGQCLEWMGYI |
| | | | NPYSDVIKYNEKFKGRVTLTSDKSTSTAYMELSSLRS |
| | | | EDTAVYYCARDSYYSYDGFAYWGQGTLVTVSSGGG |
| | | | GSGGGGSGGGGSGGGGSEIVLTQSPDFQSVTPKEKVT |
| | | | ITCSASSSVSYMHWYQQKPDQSPKRLIYDTSKLASGV |
| | | | PSRFSGSGSGTDFTLTINSLEAEDAATYYCQQWSSNP |
| | | | PTFGCGTKVEIKGGGGSGGGGSDKTHTCPPCPAPEAA |
| | | | GGPSVFLFPPKPKDTLMISRTPEVTCVVVDVSHEDPE |
| | | | VKFNWYVDGVEVHNAKTKPREEQYNSTYRVVSVLT |
| | | | VLHQDWLNGKEYKCKVSNKALPAPIEKTISKAKGQP |
| | | | REPQVCTLPPSREEMTKNQVSLSCAVKGFYPSDIAVE |
| | | | WESNGQPENNYKTTPPVLDSDGSFFLVSKLTVDKSR |
| | | | WQQGNVFSCSVMHEALHNHYTQKSLSLSPGK |
| 286 | | light | EIVLTQSPATLSLSPGERATLSCRASQSVSSYLAWYQ |
| | | chain | QKPGQAPRLLIYDASNRATGIPARFSGSGSGTDFTLTI |
| | | | SSLEPEDFAVYYCQQRSNWPITFGQGTRLEIKRTVAA |
| | | | PSVFIFPPSDEQLKSGTASVVCLLNNFYPREAKVQWK |
| | | | VDNALQSGNSQESVTEQDSKDSTYSLSSTLTLSKADY |
| | | | EKHKVYACEVTHQGLSSPVTKSFNRGEC |
| 296 | ATG201- | heavy | QVQLQQPGAELVKPGASVKMSCKASGYTFTSYNMH |
| | Y101F- | chain | WVKQTPGRGLEWIGAIYPGNGDTSYNQKFKGKATL |
| | 20R | 1 | TADKSSSTAYMQLSSLTSEDSAVYYCARSTYYGGD |
| | | | WYFNVWGAGTTVTVSAASTKGPSVFPLAPSSKSTSG |
| | | | GTAALGCLVKDYFPEPVTVSWNSGALTSGVHTFPAV |
| | | | LQSSGLYSLSSVVTVPSSSLGTQTYICNVNHKPSNTK |
| | | | VDKKVEPKSCDKTHTCPPCPAPEAAGGPSVFLFPPKP |
| | | | KDTLMISRTPEVTCVVVDVSHEDPEVKFNWYVDGV |
| | | | EVHNAKTKPREEQYNSTYRVVSVLTVLHQDWLNGK |
| | | | EYKCKVSNKALPAPIEKTISKAKGQPREPQVYTLPPC |
| | | | REEMTKNQVSLWCLVKGFYPSDIAVEWESNGQPEN |
| | | | NYKTTPPVLDSDGSFFLYSKLTVDKSRWQQGNVFSC |
| | | | SVMHEALHNHYTQKSLSLSPGK |
| 297 | | heavy | QVQLQQPGAELVKPGASVKMSCKASGYTFTSYNMH |
| | | chain | WVKQTPGRGLEWIGAIYPGNGDTSYNQKFKGKATL |
| | | 2 | TADKSSSTAYMQLSSLTSEDSAVYYCARSTYYGGD |
| | | | WYFNVWGAGTTVTVSAASTKGPSVFPLAPSSKSTSG |
| | | | GTAALGCLVKDYFPEPVTVSWNSGALTSGVHTFPAV |
| | | | LQSSGLYSLSSVVTVPSSSLGTQTYICNVNHKPSNTK |
| | | | VDKKVEPKSCGGGGSGGGGSEVQLVQSGAEVKKPG |
| | | | SSVKVSCKASGYTFSSYVIHWVRQAPGQCLEWMGYI |
| | | | NPYNDVIKYNEKFKGRVTLTSDKSTSTAYMELSSLRS |
| | | | EDTAVYYCARDSFYSYDGFAYWGQGTLVTVSSGGG |
| | | | GSGGGGSGGGGSGGGGSEIVLTQSPDFQSVTPKEKVT |
| | | | ITCSASSSVSYMHWYQQKPDQSPKRLIYDTSKLASGV |
| | | | PSRFSGSGSGTDFTLTINSLEAEDAATYYCQQWSSNP |
| | | | PTFGCGTKVEIKGGGGSGGGGSDKTHTCPPCPAPEAA |
| | | | GGPSVFLFPPKPKDTLMISRTPEVTCVVVDVSHEDPE |
| | | | VKFNWYVDGVEVHNAKTKPREEQYNSTYRVVSVLT |
| | | | VLHQDWLNGKEYKCKVSNKALPAPIEKTISKAKGQP |
| | | | REPQVCTLPPSREEMTKNQVSLSCAVKGFYPSDIAVE |
| | | | WESNGQPENNYKTTPPVLDSDGSFFLVSKLTVDKSR |
| | | | WQQGNVFSCSVMHEALHNHYTQKSLSLSPGK |
| 298 | | light | QIVLSQSPAILSASPGEKVTMTCRASSSVSYIHWFQQ |
| | | chain | KPGSSPKPWIYATSNLASGVPVRFSGSGSGTSYSLTIS |
| | | | RVEAEDAATYYCQQWTSNPPTFGGGTKLEIKRTVAA |
| | | | PSVFIFPPSDEQLKSGTASVVCLLNNFYPREAKVQWK |
| | | | VDNALQSGNSQESVTEQDSKDSTYSLSSTLTLSKADY |
| | | | EKHKVYACEVTHQGLSSPVTKSFNRGEC |
| 296 | ATG201- | heavy | QVQLQQPGAELVKPGASVKMSCKASGYTFTSYNMH |
| | N55S- | chain | WVKQTPGRGLEWIGAIYPGNGDTSYNQKFKGKATL |
| | 20R | 1 | TADKSSSTAYMQLSSLTSEDSAVYYCARSTYYGGD |
| | | | WYFNVWGAGTTVTVSAASTKGPSVFPLAPSSKSTSG |
| | | | GTAALGCLVKDYFPEPVTVSWNSGALTSGVHTFPAV |
| | | | LQSSGLYSLSSVVTVPSSSLGTQTYICNVNHKPSNTK |
| | | | VDKKVEPKSCDKTHTCPPCPAPEAAGGPSVFLFPPKP |
| | | | KDTLMISRTPEVTCVVVDVSHEDPEVKFNWYVDGV |
| | | | EVHNAKTKPREEQYNSTYRVVSVLTVLHQDWLNGK |
| | | | EYKCKVSNKALPAPIEKTISKAKGQPREPQVYTLPPC |
| | | | REEMTKNQVSLWCLVKGFYPSDIAVEWESNGQPEN |
| | | | NYKTTPPVLDSDGSFFLYSKLTVDKSRWQQGNVFSC |
| | | | SVMHEALHNHYTQKSLSLSPGK |
| 299 | | heavy | QVQLQQPGAELVKPGASVKMSCKASGYTFTSYNMH |
| | | chain | WVKQTPGRGLEWIGAIYPGNGDTSYNQKFKGKATL |
| | | 2 | TADKSSSTAYMQLSSLTSEDSAVYYCARSTYYGGD |
| | | | WYFNVWGAGTTVTVSAASTKGPSVFPLAPSSKSTSG |
| | | | GTAALGCLVKDYFPEPVTVSWNSGALTSGVHTFPAV |
| | | | LQSSGLYSLSSVVTVPSSSLGTQTYICNVNHKPSNTK |
| | | | VDKKVEPKSCGGGGSGGGGSEVQLVQSGAEVKKPG |
| | | | SSVKVSCKASGYTFSSYVIHWVRQAPGQCLEWMGYI |
| | | | NPYSDVIKYNEKFKGRVTLTSDKSTSTAYMELSSLRS |
| | | | EDTAVYYCARDSYYSYDGFAYWGQGTLVTVSSGGG |
| | | | GSGGGGSGGGGSGGGGSEIVLTQSPDFQSVTPKEKVT |
| | | | ITCSASSSVSYMHWYQQKPDQSPKRLIYDTSKLASGV |
| | | | PSRFSGSGSGTDFTLTINSLEAEDAATYYCQQWSSNP |
| | | | PTFGCGTKVEIKGGGGSGGGGSDKTHTCPPCPAPEAA |
| | | | GGPSVFLFPPKPKDTLMISRTPEVTCVVVDVSHEDPE |
| | | | VKFNWYVDGVEVHNAKTKPREEQYNSTYRVVSVLT |
| | | | VLHQDWLNGKEYKCKVSNKALPAPIEKTISKAKGQP |
| | | | REPQVCTLPPSREEMTKNQVSLSCAVKGFYPSDIAVE |
| | | | WESNGQPENNYKTTPPVLDSDGSFFLVSKLTVDKSR |
| | | | WQQGNVFSCSVMHEALHNHYTQKSLSLSPGK |
| 298 | | light | QIVLSQSPAILSASPGEKVTMTCRASSSVSYIHWFQQ |
| | | chain | KPGSSPKPWIYATSNLASGVPVRFSGSGSGTSYSLTIS |
| | | | RVEAEDAATYYCQQWTSNPPTFGGGTKLEIKRTVAA |
| | | | PSVFIFPPSDEQLKSGTASVVCLLNNFYPREAKVQWK |
| | | | VDNALQSGNSQESVTEQDSKDSTYSLSSTLTLSKADY |
| | | | EKHKVYACEVTHQGLSSPVTKSFNRGEC |
| 272 | ATG201- | heavy | QVQLQQSGAELVRPGSSVKISCKASGYAFSSYWMN |
| | D105R- | chain | WVKQRPGQGLEWIGQIWPGDGDTNYNGKFKGKATL |
| | 19B | 1 | TADESSSTAYMQLSSLASEDSAVYFCARRETTTVGR |
| | | | YYYAMDYWGQGTTVTVSSASTKGPSVFPLAPSSKST |
| | | | SGGTAALGCLVKDYFPEPVTVSWNSGALTSGVHTFP |
| | | | AVLQSSGLYSLSSVVTVPSSSLGTQTYICNVNHKPSN |
| | | | TKVDKKVEPKSCDKTHTCPPCPAPEAAGGPSVFLFPP |
| | | | KPKDTLMISRTPEVTCVVVDVSHEDPEVKFNWYVDG |
| | | | VEVHNAKTKPREEQYNSTYRVVSVLTVLHQDWLNG |
| | | | KEYKCKVSNKALPAPIEKTISKAKGQPREPQVYTLPP |
| | | | CREEMTKNQVSLWCLVKGFYPSDIAVEWESNGQPE |
| | | | NNYKTTPPVLDSDGSFFLYSKLTVDKSRWQQGNVFS |
| | | | CSVMHEALHNHYTQKSLSLSPGK |
| 300 | | heavy | QVQLQQSGAELVRPGSSVKISCKASGYAFSSYWMN |
| | | chain | WVKQRPGQGLEWIGQIWPGDGDTNYNGKFKGKATL |
| | | 2 | TADESSSTAYMQLSSLASEDSAVYFCARRETTTVGR |
| | | | YYYAMDYWGQGTTVTVSSASTKGPSVFPLAPSSKST |
| | | | SGGTAALGCLVKDYFPEPVTVSWNSGALTSGVHTFP |
| | | | AVLQSSGLYSLSSVVTVPSSSLGTQTYICNVNHKPSN |
| | | | TKVDKKVEPKSCGGGGSGGGGSEVQLVQSGAEVKK |
| | | | PGSSVKVSCKASGYTFSSYVIHWVRQAPGQCLEWM |
| | | | GYINPYNDVIKYNEKFKGRVTLTSDKSTSTAYMELSS |
| | | | LRSEDTAVYYCARDSYYSYRGFAYWGQGTLVTVSS |
| | | | GGGGSGGGGSGGGGSGGGGSEIVLTQSPDFQSVTPKE |
| | | | KVTITCSASSSVSYMHWYQQKPDQSPKRLIYDTSKL |
| | | | ASGVPSRFSGSGSGTDFTLTINSLEAEDAATYYCQQW |
| | | | SSNPPTFGCGTKVEIKGGGGSGGGGSDKTHTCPPCPA |
| | | | PEAAGGPSVFLFPPKPKDTLMISRTPEVTCVVVDVSH |
| | | | EDPEVKFNWYVDGVEVHNAKTKPREEQYNSTYRVV |
| | | | SVLTVLHQDWLNGKEYKCKVSNKALPAPIEKTISKA |
| | | | KGQPREPQVCTLPPSREEMTKNQVSLSCAVKGFYPS |
| | | | DIAVEWESNGQPENNYKTTPPVLDSDGSFFLVSKLTV |
| | | | DKSRWQQGNVFSCSVMHEALHNHYTQKSLSLSPGK |
| 274 | | light | DIQLTQSPASLAVSLGQRATISCKASQSVDYDGDSYL |
| | | chain | NWYQQIPGQPPKLLIYDASNLVSGIPPRFSGSGSGTDF |
| | | | TLNIHPVEKVDAATYHCQQSTEDPWTFGGGTKLEIK |
| | | | RTVAAPSVFIFPPSDEQLKSGTASVVCLLNNFYPREA |
| | | | KVQWKVDNALQSGNSQESVTEQDSKDSTYSLSSTLT |
| | | | LSKADYEKHKVYACEVTHQGLSSPVTKSFNRGEC |

10.2 CD19×CD3 and CD20×CD3 Bispecific Antibody Characterization: Binding Affinity to NALM6 Cells, Raji cells, Primary B cells, DoHH2 Cells, SU-DHL-1 Cells and Jurkat Cells

To evaluate the binding affinity of the generated bispecific antibodies to NALM6 cells, Raji cells, Primary B cells, DoHH2 cells, SU-DHL-1 cells and Jurkat cells, the NALM6 cells expressing CD19, Raji cells expressing CD19, Primary B cells, DoHH2 cells expressing CD20, SU-DHL-1 cells without expressing CD19 or CD20 and Jurkat cells expressing CD3 were stained by the test antibodies and the binding affinity was determined by FACS analysis. Primary B cells were isolated from healthy donors' PBMC by human B cell enrichment Kit (Stemcell #19054). The protocol of FACS analysis is described as follows:
(a) The cells from each cell line above were harvested and centrifuged at 300 g for 5 min, and the supernatant were discarded.
(b) The cells were washed 2 times with FACS buffer.
(c) Step (a) was repeated.
(d) The cells were resuspended and seeded at 1*10⁵ cell/well into the assay plate in 50 μl FACS buffer, and then 50 μl primary antibody (primary antibody final concentration: 100.000, 33.333, 11.111, 3.704, 1.235, 0.412, 0.137, 0.046, 0.000 nM, or 200.000, 20.000, 4.000, 0.800, 0.160, 0.032, 0.006, 0.001, 0 nM, or 500, 250, 125, 63, 31, 16, 8, 4, 0 nM) was added to each well, and incubated at CO₂ incubator for 1 hour.
(e) Step (b) was repeated and the cells were resuspended with 100 μl/well diluted secondary antibody, and incubated at 4° C. for 45 minutes in the dark.
(f) Step (b) was repeated and the cells were resuspended the cells with 150 μl/well FACS buffer and kept in dark for FACS analysis.

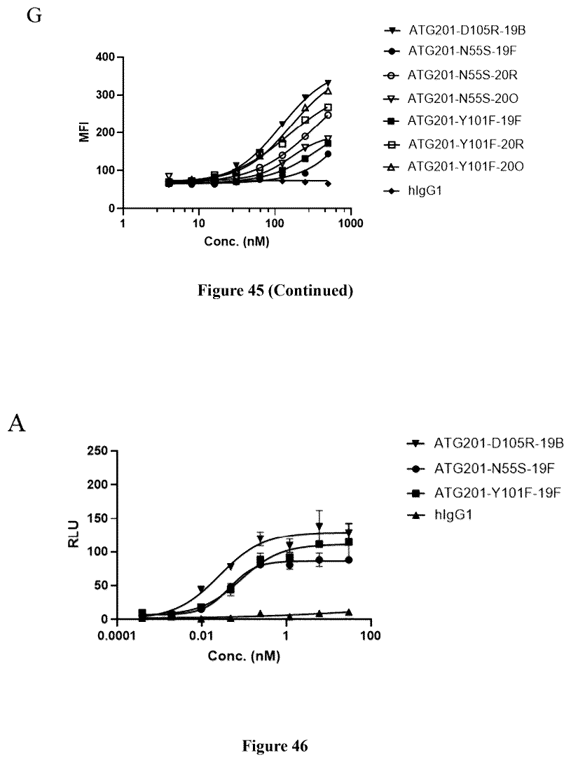

As shown in FIG. 45A, ATG201-Y101F-19F, ATG201-N55S-19F and ATG201-D105R-19B showed binding ability to NALM6 cells with a half maximal effective concentration (EC₅₀) of 4.359 nM, 4.913 nM and 2.898 nM respectively. In another experiment, ATG201-N55S-19F, ATG201-Y101F-19F, ATG201-N55S-19B and ATG201-Y101F-19B, showed binding ability to NALM6 cells with an EC₅₀ of 0.847 nM, 0.609 nM, 0.513 and 0.508 nM respectively (FIG. 45B). As shown in FIG. 45C, ATG201-N55S-19F, ATG201-Y101F-19F, ATG201-N55S-19B and ATG201-Y101F-19B showed binding ability to Raji cells with an EC₅₀ of 1.495 nM, 1.126 nM, 1.108 nM and 1.108 nM respectively. As shown in FIG. 45D, ATG201-N55S-19F, ATG201-Y101F-19F, ATG201-N55S-19B and ATG201-Y101F-19B showed binding ability to Primary B cells with an EC₅₀ of 1.085 nM, 0.468 nM, 0.442 nM and 0.359 nM respectively. As shown in FIG. 45E, ATG201-Y101F-200, ATG201-Y101F-20R, ATG201-N55S-200 and ATG201-N55S-20R showed binding ability to DoHH2 cells with an EC₅₀ of 7.547 nM, 4.049 nM, 4.894 nM and 3.134 nM respectively. These bispecific antibodies did not bind to SU-DHL-1 cells (FIG. 45F). In addition, the generated bispecific antibodies showed limited or weak CD3 binding on Jurkat T cells (FIG. 45G).

10.3 CD19×CD3 and CD20×CD3 Bispecific Antibodies Characterization: T Cell Activation with Jurkat NFAT Reporter Cell Line

The T cell activation capability of these Fab-scFv-Fab bispecific antibodies was tested by Jurkat-NFAT-Luciferase assay, wherein NALM6, DoHH2, SU-DHL-1 were used as target cell and Jurkat was used as effector cell. In this assay, 2.5*10⁴ cell/well target cells were seeded into the assay plate and incubated with 5*10⁴ cell/well of Jurkat-NFAT-Luc cells and a serial concentration of bispecific antibodies in CO₂ incubator for 24 hours. Cells were collected and luciferase (Luc) expression level was evaluated by Luciferase Assay System (Promega #E4530).

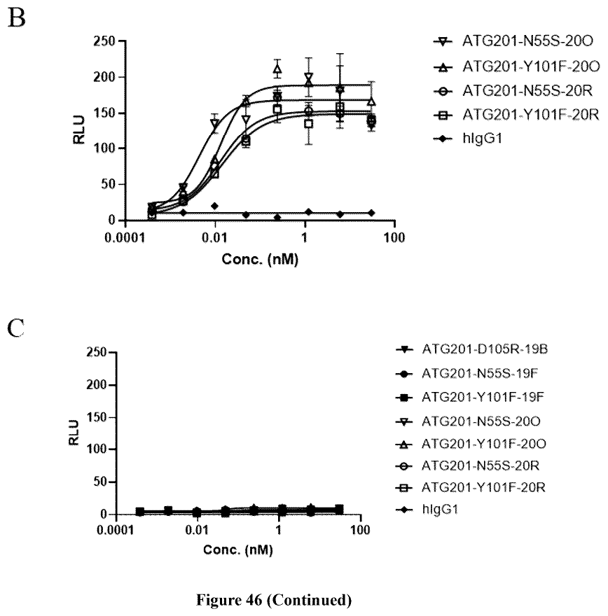

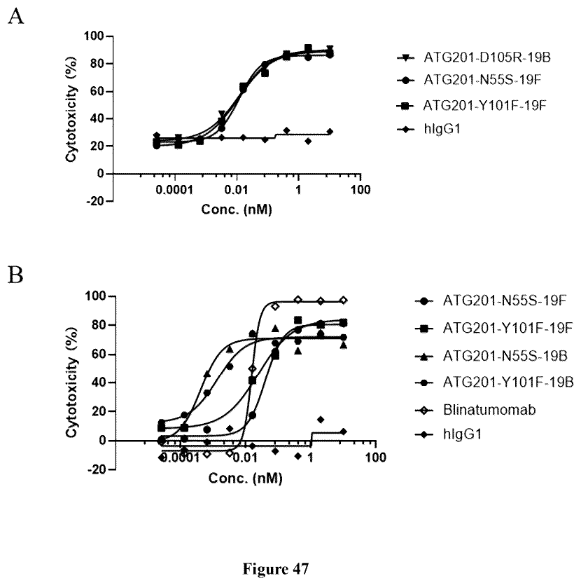

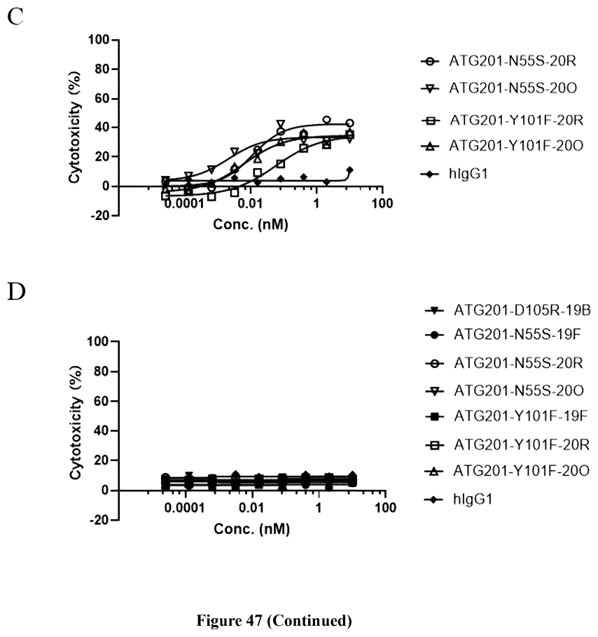

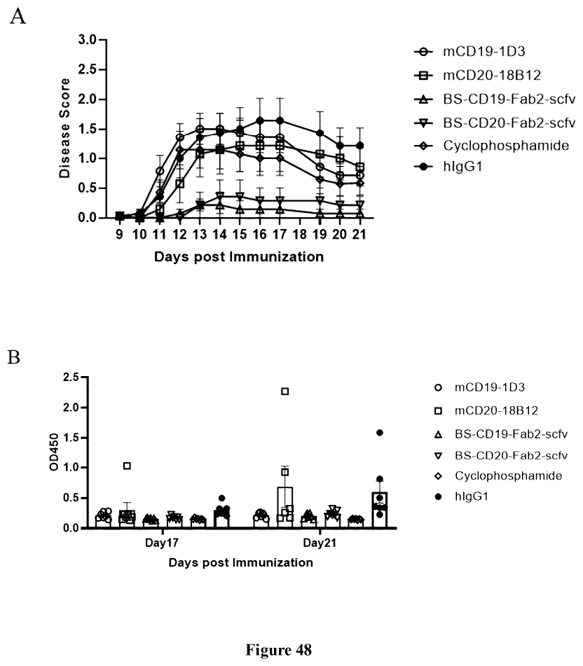

As shown in FIG. 46, ATG201-Y101F-19F, ATG201-N55S-19F and ATG201-D105R-19B induced Jurkat T cell activation in the presence of CD19-expressing NALM6 cells (FIG. 46A), but not in the presence of CD19-negative SU-DHL-1 cells (FIG. 46C). ATG201-Y101F-200, ATG201-Y101F-20R, ATG201-N55S-200 and ATG201-N55S-20R induced Jurkat T cell activation in the presence of CD20-expressing DoHH2 cells (FIG. 46B), but not in the presence of CD20-negative SU-DHL-1 cells (FIG. 46C).

### 10.4 CD19×CD3 and CD20×CD3 Bispecific Antibodies Characterization: T-Cell Dependent Cytotoxicity

The cytotoxicity of these Fab-scFv-Fab bispecific antibodies on different tumor cell lines (NALM6, DoHH2) and CD19 and CD20 negative expressed cells (SU-DHL-1) were tested by FACS analysis. T cells were isolated from healthy donors' PBMC by human T cell enrichment Kit (Stemcell #19051). 2*10⁴ cell/well target cells labelled with CFSE were seeded into the assay plate and incubated with 1*10⁵ cell/well of T cells in the presence or absence of diluted antibodies in CO2 incubator for 24 hours. Cells were harvested and tumor cell killing was evaluated by flow cytometry.

As shown in FIG. 47, ATG201-Y101F-19F, ATG201-N55S-19F, ATG201-N55S-19B, ATG201-Y101F-19B and ATG201-D105R-19B mediated T cell cytotoxicity in the presence of CD19-expressing NALM6 cells (FIG. 47A), but not in the presence of CD19-negative SU-DHL-1 cells (FIG. 47D). ATG201-Y101F-200, ATG201-Y101F-20R, ATG201-N55S-200 and ATG201-N55S-20R mediated T cell cytotoxicity in the presence of CD20-expressing DoHH2 cells (FIG. 47C), but not in the presence of CD20-negative SU-DHL-1 cells (FIG. 47D). ATG201-N55S-19B and ATG201-Y101F-19B were more potent than the benchmark Blinatumomab (CD19×CD3 BiTE) in mediating lysis of NALM6 cells (FIG. 47B).

10.5 CD19×CD3 and CD20×CD3 Bispecific Antibodies Characterization: In vivo Efficacy in Myelin Oligodendrocyte of Glycoprotein-Induced Experimental Autoimmune Encephalomyelitis (MOG-EAE) Model

To assess the disease progression suppressing efficacy of the CD19×CD3 or CD20×CD3 bispecific antibody on myelin oligodendrocyte in vivo, glycoprotein-induced experimental autoimmune encephalomyelitis (MOG-EAE) model was used. C57BL/6 mice (10 weeks, female) were injected subcutaneously (s.c.) with 100 μg recombinant human MOG (rhMOG) in complete Freund's adjuvant (CFA) containing 5 mg/ml mycobacteria. Mice were injected intravenously (i.v.) with 200 ng pertussis toxin on day 0 and day 2. 7 female animals per group were randomized to receive either 10 mg/kg anti-mouse CD19 monoclonal antibody (mCD19-1D3), 10 mg/kg anti-mouse CD20 monoclonal antibody (mCD20-18B12), 1 mg/kg surrogated CD19×CD3 bispecific antibody (BS-CD19-Fab2-scFv), 1 mg/kg surrogated CD20×CD3 bispecific antibody (BS-CD20-Fab2-scFv), 40 mg/kg Cyclophosphamide (positive control) and 1 mg/kg Ig Gl isotype by intraperitionally (i.p.) twice a week. The plasma samples were collected at 2 hours post-first dose on day 10 for cytokine release analysis using the LEGENDplex™ Mouse Th1 Panel kit (biolegend #741050). The plasma samples were collected on Day 17 and 21 for anti-MOG testing. Clinical disease was assessed as follows: 0, no clinical signs; 0.5, Tip of tail is limp; 1.0, Limp tail; 1.5, Limp tail and hind leg inhibition; 2.0, Limp tail and weakness of hind legs; 2.5, Limp tail and dragging of hind legs; 3.0, Limp tail and complete paralysis of hind legs; 3.5, In addition to limp tail and complete paralysis of hind legs, hind legs are together on one side of body or the hind quarters are flat like a pancake, giving the appearance of a hump in the front quarters of the mice; 4.0, Limp tail, complete hind leg and partial front leg paralysis; 4.5, Complete hind and partial front leg paralysis, no movement around the cage; 5.0, Dead due to paralysis or euthanized due to severe paralysis (Often euthanasia is recommended after the mouse scores 4.0 for 2 days).

The mice of each group were anaesthetized i.p. with 800 mg/kg Tribromoethyl alcohol and perfused with PBS for 10 min, followed by 4% paraformaldehyde for the next 10 min on Day 21. Spinal cords were carefully removed from the vertebral canal and postfixed in the same fixative. The tissue samples were then embedded in paraffin and were sectioned (4 μm). The sections were stained with haematoxylin and eosin (H&E) for pathological observation of inflammation infiltrating. The inflammation infiltrating in spinal cords were scored on a scale from 0 to 3 (0, no changes; 3, severe inflammatory cell infiltration).

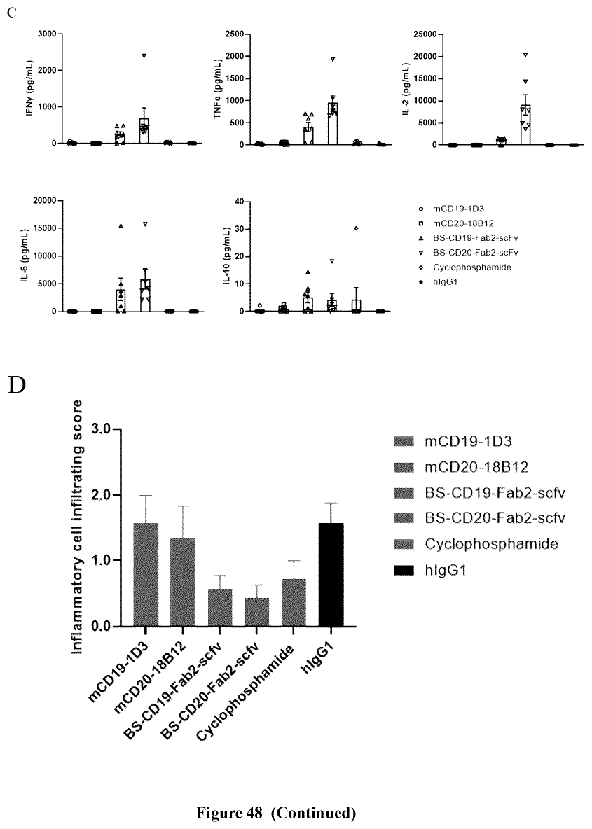

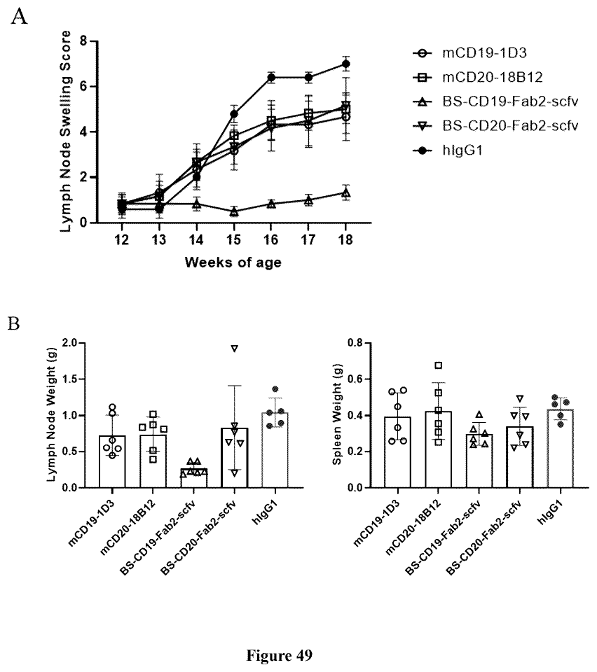

As shown in FIG. 48A, the surrogated CD19×CD3 bispecific antibody demonstrated enhanced efficacy in suppressing disease progression than CD20×CD3 bispecific antibody, and CD19 or CD20 monoclonal antibody in MOG-EAE mice. The surrogated CD19×CD3 bispecific antibody, CD19 and CD20 monoclonal antibody reduced anti-MOG production on Day 17 and 21 post-immunization (FIG. 48B). The surrogated bispecific antibodies induced inflammatory cytokine release, but not monoclonal antibodies. The surrogated CD20×CD3 bispecific antibody showed much higher cytokine release than CD19×CD3 bispecific antibody (FIG. 48C). In addition, the surrogated CD19×CD3 or CD20×CD3 bispecific antibody showed lower inflammatory cell infiltration than CD19 or CD20 monoclonal antibody (FIG. 48D). The amino acid sequences of VH and VL regions of antibodies mCD19-1D3 and mCD20-18B12, as well as the amino acid sequences of VH and VL regions of the scFv domain targeting CD3 are shown in Table 49 below, and CDR sequences identified by the convention of Kabat are bold and underlined in each sequence listed in Table 49 below.

TABLE 49
Amino Acid Sequences of VH, VL, heavy chain and light chain of
mCD19-1D3, mCD20-18B12 and mCD3-2C11

| | | | |
|---|---|---|---|
| mCD19- | VH | 313 | EVQLQQSGAELVRPGTSVKLSCKVSGDTIT |
| 1D3 | | | FYYMHFVKQRPGQGLEWIGRIDPEDEST |
| | | | KYSEKFKNKATLTADTSSNTAYLKLSSLTS |
| | | | EDTATYFCIYGGYYFDYWGQGVMVTVSS |
| | VL | 314 | DIQMTQSPASLSTSLGETVTIQCQASEDIYS |
| | | | GLAWYQQKPGKSPQLLIYGASDLQDGVP |
| | | | SRFSGSGSGTQYSLKITSMQTEDEGVYFCQ |
| | | | QGLTYPRTFGGGTKLELK |
| | Heavy | 331 | EVQLQQSGAELVRPGTSVKLSCKVSGDTIT |
| | chain | | FYYMHFVKQRPGQGLEWIGRIDPEDESTK |
| | | | YSEKFKNKATLTADTSSNTAYLKLSSLTSE |
| | | | DTATYFCIYGGYYFDYWGQGVMVTVSS |
| | | | AKTTAPSVYPLAPVCGDTTGSSVTLGCLV |
| | | | KGYFPEPVTLTWNSGSLSSGVHTFPAVLQS |
| | | | DLYTLSSSVTVTSSTWPSQSITCNVAHPASS |
| | | | TKVDKKIEPRGPTIKPCPPCKCPAPNLLGGP |
| | | | DVFIFPPKIKDVLMISLSPIVTCVVVDVSED |
| | | | DPDVQISWFVNNVEVHTAQTQTHREDYN |
| | | | STLRVVSALPIQHQDWMSGKEFKCKVNN |
| | | | KDLPLPEERTISKPKGSVRAPQVYVLPPPE |
| | | | EEMTKKQVTLTCMVTDFMPEDIYVEWTN |
| | | | NGKTELNYKNTEPVLDSDGSYFMYSKLRV |
| | | | EKKNWVERNSYSCSVVHEGLHNHHTTKS |
| | | | FSRTPGK |
| | Light | 332 | DIQMTQSPASLSTSLGETVTIQCQASEDIYS |
| | chain | | GLAWYQQKPGKSPQLLIYGASDLQDGVPS |
| | | | RFSGSGSGTQYSLKITSMQTEDEGVYFCQ |
| | | | QGLTYPRTFGGGTKLELKRADAAPTVSIFP |
| | | | PSSEQLTSGGASVVCFLNNFYPKDINVKW |
| | | | KIDGSERQNGVLNSWTDQDSKDSTYSMSS |
| | | | TLTLTKDEYERHNSYTCEATHKTSTSPIVK |
| | | | SFNRNEC |
| mCD20- | VH | 326 | QVQLQQPGAELVRPGTSVKLSCKASGYTF |
| 18B12 | | | TSYWMHWIKQRPGQGLEWIGVIDPSDNY |
| | | | TKYNQKFKGKATLTVDTSSSTAYMQLSSL |
| | | | TSEDSAVYFCAREGYYGSSPWFAYWGQG |
| | | | TLVTVSS |
| | VL | 327 | QIVMSQSPAILSASPGEKVTMTCRARSSVS |
| | | | YIHWYQQKPGSSPKPWIYATSNLASGVPG |
| | | | RFSGSGSGTSYSLTITRVEAEDAATYYCQQ |
| | | | WSSKPPTFGGGTKLEIK |
| | Heavy | 333 | QVQLQQPGAELVRPGTSVKLSCKASGYTF |
| | chain | | TSYWMHWIKQRPGQGLEWIGVIDPSDNY |
| | | | TKYNQKFKGKATLTVDTSSSTAYMQLSSLT |
| | | | SEDSAVYFCAREGYYGSSPWFAYWGQGTL |
| | | | VTVSSAKTTAPSVYPLAPVCGDTTGSSVTL |
| | | | GCLVKGYFPEPVTLTWNSGSLSSGVHTFPA |
| | | | VLQSDLYTLSSSVTVTSSTWPSQSITCNVA |
| | | | HPASSTKVDKKIEPRGPTIKPCPPCKCPAPN |
| | | | LLGGPDVFIFPPKIKDVLMISLSPIVTCVVV |
| | | | DVSEDDPDVQISWFVNNVEVHTAQTQTH |
| | | | REDYNSTLRVVSALPIQHQDWMSGKEFKC |
| | | | KVNNKDLPLPEERTISKPKGSVRAPQVYV |
| | | | LPPPEEEMTKKQVTLTCMVTDFMPEDIYV |
| | | | EWTNNGKTELNYKNTEPVLDSDGSYFMY |
| | | | SKLRVEKKNWVERNSYSCSVVHEGLHNH |
| | | | HTTKSFSRTPGK |
| | Light | 262 | QIVMSQSPAILSASPGEKVTMTCRARSSVS |
| | chain | | YIHWYQQKPGSSPKPWIYATSNLASGVPG |
| | | | RFSGSGSGTSYSLTITRVEAEDAATYYCQQ |
| | | | WSSKPPTFGGGTKLEIKRADAAPTVSIFPPS |
| | | | SEQLTSGGASVVCFLNNFYPKDINVKWKI |
| | | | DGSERQNGVLNSWTDQDSKDSTYSMSST |
| | | | LTLTKDEYERHNSYTCEATHKTSTSPIVKSF |
| | | | NRNEC |
| mCD3- | VH | 315 | EVQLVESGGGLVQPGKSLKLSCEASGFTFS |
| 2C11 | | | GYGMHWVRQAPGRCLESVAYITSSSINIK |
| | | | YADAVKGRFTVSRDNAKNLLFLQMNILKS |
| | | | EDTAMYYCARFDWDKNYWGQGTMVTV |
| | | | SS |
| | VL | 316 | DIQMTQSPSSLPASLGDRVTINCQASQDIS |
| | | | NYLNWYQQKPGKAPKLLIYYTNKLADGV |
| | | | PSRFSGSGSGRDSSFTISSLESEDIGSYYCQ |
| | | | QYYNYPWTFGCGTKLEIK |

TABLE 57
Sequence information of each generated 2 + 1 bispecific antibodies

| SEQ ID | Antibody | | |
|---|---|---|---|
| No | Code | Name | Amino Acid Sequence |
| 317 | BS- | heavy | EVQLQQSGAELVRPGTSVKLSCKVSGDTITFYY |
| | CD19- | chain 1 | MHFVKQRPGQGLEWIGRIDPEDESTKYSEKFKN |
| | Fab2- | | KATLTADTSSNTAYLKLSSLTSEDTATYFCIYGG |
| | scFv | | YYFDYWGQGVMVTVSSASTKGPSVFPLAPSSKS |
| | | | TSGGTAALGCLVKDYFPEPVTVSWNSGALTSGV |
| | | | HTFPAVLQSSGLYSLSSVVTVPSSSLGTQTYICNV |
| | | | NHKPSNTKVDKKVEPKSCDKTHTCPPCPAPEAA |
| | | | GGPSVFLFPPKPKDTLMISRTPEVTCVVVDVSHE |
| | | | DPEVKFNWYVDGVEVHNAKTKPREEQYNSTYR |
| | | | VVSVLTVLHQDWLNGKEYKCKVSNKALPAPIE |
| | | | KTISKAKGQPREPQVYTLPPCREEMTKNQVSLW |
| | | | CLVKGFYPSDIAVEWESNGQPENNYKTTPPVLD |
| | | | SDGSFFLYSKLTVDKSRWQQGNVFSCSVMHEAL |
| | | | HNHYTQKSLSLSPGK |
| 318 | | heavy | EVQLQQSGAELVRPGTSVKLSCKVSGDTITFYY |
| | | chain 2 | MHFVKQRPGQGLEWIGRIDPEDESTKYSEKFKN |
| | | | KATLTADTSSNTAYLKLSSLTSEDTATYFCIYGG |
| | | | YYFDYWGQGVMVTVSSASTKGPSVFPLAPSSKS |
| | | | TSGGTAALGCLVKDYFPEPVTVSWNSGALTSGV |
| | | | HTFPAVLQSSGLYSLSSVVTVPSSSLGTQTYICNV |
| | | | NHKPSNTKVDKKVEPKSCGGGGSGGGGSEVQL |
| | | | VESGGGLVQPGKSLKLSCEASGFTFSGYGMHW |
| | | | VRQAPGRCLESVAYITSSSINIKYADAVKGRFTV |
| | | | SRDNAKNLLFLQMNILKSEDTAMYYCARFDWD |
| | | | KNYWGQGTMVTVSSGGGGSGGGGSGGGGSGG |
| | | | GGSDIQMTQSPSSLPASLGDRVTINCQASQDISN |
| | | | YLNWYQQKPGKAPKLLIYYTNKLADGVPSRFSG |
| | | | SGSGRDSSFTISSLESEDIGSYYCQQYYNYPWTF |
| | | | GCGTKLEIKGGGGSGGGGSDKTHTCPPCPAPEA |
| | | | AGGPSVFLFPPKPKDTLMISRTPEVTCVVVDVSH |
| | | | EDPEVKFNWYVDGVEVHNAKTKPREEQYNSTY |
| | | | RVVSVLTVLHQDWLNGKEYKCKVSNKALPAPIE |
| | | | KTISKAKGQPREPQVCTLPPSREEMTKNQVSLS |
| | | | CAVKGFYPSDIAVEWESNGQPENNYKTTPPVLD |
| | | | SDGSFFLVSKLTVDKSRWQQGNVFSCSVMHEAL |
| | | | HNHYTQKSLSLSPGK |
| 319 | | light | DIQMTQSPASLSTSLGETVTIQCQASEDIYSGLA |
| | | chain | WYQQKPGKSPQLLIYGASDLQDGVPSRFSGSGS |
| | | | GTQYSLKITSMQTEDEGVYFCQQGLTYPRTFGG |
| | | | GTKLELKRTVAAPSVFIFPPSDEQLKSGTASVVC |
| | | | LLNNFYPREAKVQWKVDNALQSGNSQESVTEQ |
| | | | DSKDSTYSLSSTLTLSKADYEKHKVYACEVTHQ |
| | | | GLSSPVTKSFNRGEC |
| 328 | BS- | heavy | QVQLQQPGAELVRPGTSVKLSCKASGYTFTSYW |
| | CD20- | chain 1 | MHWIKQRPGQGLEWIGVIDPSDNYTKYNQKFK |
| | Fab2- | | GKATLTVDTSSSTAYMQLSSLTSEDSAVYFCAR |
| | scFv | | EGYYGSSPWFAYWGQGTLVTVSSASTKGPSVFP |
| | | | LAPSSKSTSGGTAALGCLVKDYFPEPVTVSWNS |
| | | | GALTSGVHTFPAVLQSSGLYSLSSVVTVPSSSLG |
| | | | TQTYICNVNHKPSNTKVDKKVEPKSCDKTHTCP |
| | | | PCPAPEAAGGPSVFLFPPKPKDTLMISRTPEVTCV |
| | | | VVDVSHEDPEVKFNWYVDGVEVHNAKTKPREE |
| | | | QYNSTYRVVSVLTVLHQDWLNGKEYKCKVSNK |
| | | | ALPAPIEKTISKAKGQPREPQVYTLPPCREEMTK |
| | | | NQVSLWCLVKGFYPSDIAVEWESNGQPENNYK |
| | | | TTPPVLDSDGSFFLYSKLTVDKSRWQQGNVFSC |
| | | | SVMHEALHNHYTQKSLSLSPGK |
| 329 | | heavy | QVQLQQPGAELVRPGTSVKLSCKASGYTFTSYW |
| | | chain 2 | MHWIKQRPGQGLEWIGVIDPSDNYTKYNQKFK |
| | | | GKATLTVDTSSSTAYMQLSSLTSEDSAVYFCAR |
| | | | EGYYGSSPWFAYWGQGTLVTVSSASTKGPSVFP |
| | | | LAPSSKSTSGGTAALGCLVKDYFPEPVTVSWNS |
| | | | GALTSGVHTFPAVLQSSGLYSLSSVVTVPSSSLG |
| | | | TQTYICNVNHKPSNTKVDKKVEPKSCGGGGSGG |
| | | | GGSEVQLVESGGGLVQPGKSLKLSCEASGFTFSG |
| | | | YGMHWVRQAPGRCLESVAYITSSSINIKYADAV |
| | | | KGRFTVSRDNAKNLLFLQMNILKSEDTAMYYC |
| | | | ARFDWDKNYWGQGTMVTVSSGGGGSGGGGSG |
| | | | GGGSGGGGSDIQMTQSPSSLPASLGDRVTINCQA |
| | | | SQDISNYLNWYQQKPGKAPKLLIYYTNKLADGV |
| | | | PSRFSGSGSGRDSSFTISSLESEDIGSYYCQQYYN |
| | | | YPWTFGCGTKLEIKGGGGSGGGGSDKTHTCPPC |
| | | | PAPEAAGGPSVFLFPPKPKDTLMISRTPEVTCVV |
| | | | VDVSHEDPEVKFNWYVDGVEVHNAKTKPREEQ |
| | | | YNSTYRVVSVLTVLHQDWLNGKEYKCKVSNKA |
| | | | LPAPIEKTISKAKGQPREPQVCTLPPSREEMTKN |
| | | | QVSLSCAVKGFYPSDIAVEWESNGQPENNYKTT |
| | | | PPVLDSDGSFFLVSKLTVDKSRWQQGNVFSCSV |
| | | | MHEALHNHYTQKSLSLSPGK |
| 330 | | light | QIVMSQSPAILSASPGEKVTMTCRARSSVSYIHW |
| | | chain | YQQKPGSSPKPWIYATSNLASGVPGRFSGSGSGT |
| | | | SYSLTITRVEAEDAATYYCQQWSSKPPTFGGGT |
| | | | KLEIKRTVAAPSVFIFPPSDEQLKSGTASVVCLLN |
| | | | NFYPREAKVQWKVDNALQSGNSQESVTEQDSK |
| | | | DSTYSLSSTLTLSKADYEKHKVYACEVTHQGLS |
| | | | SPVTKSFNRGEC |

10.6 CD19×CD3 and CD20×CD3 Bispecific Antibodies Characterization: In vivo Efficacy in spontaneous MRL/lpr SLE Model

To assess the disease progression suppressing efficacy of the CD19×CD3 or CD20×CD3 bispecific antibody in vivo, spontaneous MRL/lpr SLE model was used. MRL/lpr mice were housed under specific pathogen-free conditions. When the mice started to be onset of disease (lymph node swelling score reached to about 0.8, and the age of mice is about 12 weeks), 6 animals per group were randomized to receive either 10 mg/kg anti-mouse CD19 monoclonal antibody (mCD19-1D3), 10 mg/kg anti-mouse CD20 monoclonal antibody (mCD20-18B12), 1 mg/kg surrogated CD19×CD3 bispecific antibody (BS-CD19-Fab2-scFv), 1 mg/kg surrogated CD20×CD3 bispecific antibody (BS-CD20-Fab2-scFv), and 1 mg/kg Ig G1 isotype by intraperitionally (i.p.) twice a week. The blood samples were collected at 7 days post-first dose for B cell depletion analysis by flow cytometry. The plasma samples were collected weekly for autoantibody determination by ELISA. At the end of the experiment, mice were euthanized. The tissues including spleen and lymph node were collected for weighing. The diameter of each palpable lymph node (superficial cervical, axillary, brachial and inguinal lymph node) was measured and graded as follows: 0, 0-5 mm; 1, 6-10 mm; 2, 11-15 mm; 3, 16-20 mm; 4, 21-25 mm. The total score of each mouse was the summation of the individual scores of the lymph nodes.

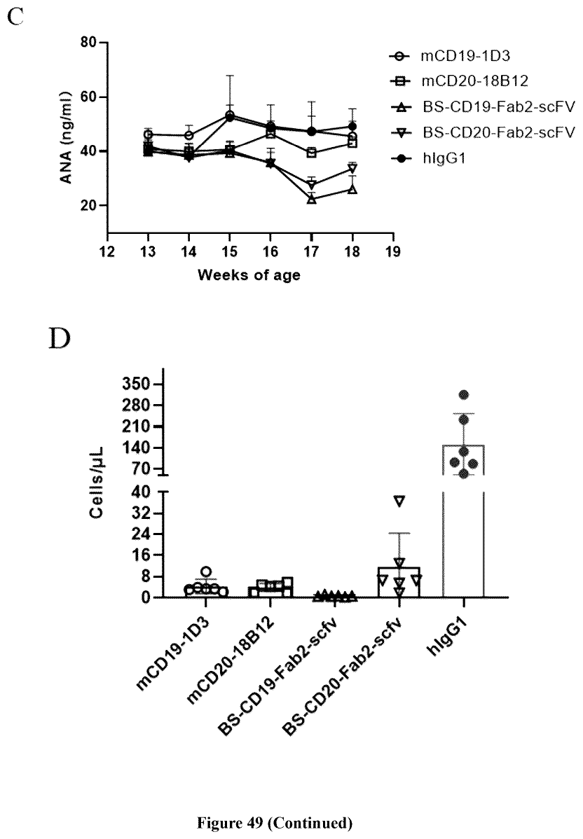

As shown in FIGS. 49A-B, the surrogated CD19×CD3 bispecific antibody demonstrated enhanced efficacy in suppressing disease progression than CD20×CD3 bispecific antibody, and CD19 or CD20 monoclonal antibody in MRL/lpr mice. The surrogated CD19×CD3 bispecific antibody decreased antinuclear antibody (ANA) production better than CD20×CD3 bispecific antibody, CD19 and CD20 monoclonal antibody (FIG. 49C). The surrogated CD19×CD3 bispecific antibody was more potent than CD20×CD3 bispecific antibody, and CD19 or CD20 monoclonal antibody in B cell depletion in peripheral blood (FIG. 49D).

What claimed is:

1. An antibody or antigen-binding fragment thereof, comprising:
i. a first binding moiety that binds to a conformational epitope of CD3, and
ii. a second binding moiety that binds to a second target other than CD3,
wherein the first binding moiety and the second binding moiety are independently a single-chain Fv (scFv) or a Fab domain.

2. The antibody or antigen-binding fragment thereof of claim 1, wherein the first binding moiety comprises
one, two or three heavy chain complementarity determining regions (HCDR1, HCDR2 and/or HCDR3) contained within SEQ ID NOs: 3, 7, 11, 21, 22, 23, 26, 27, 28, 29, 30, 80, 82, 83, 84, 85, 86, 119, 120, 122, 124, 125, 126, 128, 131, 134, 136, 138, 139, 145, 195, 196, 197, 200, 201, 202, 203, 204, 205, 207, 208, 209, 210, 211, 221, 222, 223, 224, 225, 226, 227, 228, 229, 230, 231, 232 or 315; and/or
one, two or three light chain complementarity determining regions (LCDR1, LCDR2 and/or LCDR3) contained within SEQ ID NOs: 4, 8, 12, 24, 25, 81, 87, 88, 89, 90, 91, 92, 93, 94, 95, 141, 146, 198, 199, 206, 212, 213, 214, 215, 216, 217, 218, 219, 220, 233, 234 or 316.

3. The antibody or antigen-binding fragment thereof of claim 1 or 2, wherein the first binding moiety is an anti-CD3 scFv domain, and the second binding moiety is a Fab domain; or the first binding moiety is an anti-CD3 Fab domain, and the second binding moiety is a scFv domain.

4. The antibody or antigen-binding fragment thereof of any one of claims 1 to 3, wherein the first binding moiety is linked to the second binding moiety directly or via a linker.

5. The antibody or antigen-binding fragment thereof claim 4, wherein the N-terminus of the first binding moiety is linked to the C-terminus of the second binding moiety via the linker.

6. The antibody or antigen-binding fragment thereof of claim 1 or 2, comprising, from N-terminus to C-terminus, a first chain comprising a VH region from the second binding moiety, a CH1 region, a VH region from the first binding moiety and a VL region from the first binding moiety.

7. The antibody or antigen-binding fragment thereof of claim 6, wherein the VH region from the first binding moiety and the VL region from the first binding moiety are directly linked or linked via a linker.

8. The antibody or antigen-binding fragment thereof of any one of claims 4 to 7, wherein the linker is a linker comprising glycine and serine, e.g., a (GGGGS) 2 linker.

9. The antibody or antigen-binding fragment thereof of any one of claims 6 to 8, further comprising a second chain comprising a VL region from the second binding moiety and a CL region.

10. The antibody or antigen-binding fragment thereof of any one of the preceding claims, further comprising a third binding moiety that binds to a third target other than CD3.

11. The antibody or antigen-binding fragment thereof of claim 10, wherein the second target is the same as the third target.

12. The antibody or antigen-binding fragment thereof of claim 10 or 11, wherein the third binding moiety is a Fab domain.

13. The antibody or antigen-binding fragment thereof of any one of the preceding claims, wherein the first binding moiety comprises one or two or three of HCDR1, HCDR2 and HCDR3 comprising an amino acid sequence selected from the group consisting of SEQ ID NOs: 31~40, 44, 45, 46, 50, 51, 52, 96~106, 121, 123, 127, 129, 130, 132, 133, 135, 137, 140, 143, 144, 182, 183, 184, 307, 308 and 309.

14. The antibody or antigen-binding fragment thereof of any one of the preceding claims, wherein the first binding moiety comprises one or two or three of LCDR1, LCDR2 and LCDR3 comprising an amino acid sequence selected from the group consisting of SEQ ID NOs: 41, 42, 43, 47, 48, 49, 53, 54, 55, 107~118, 142, 149, 185, 186, 187, 310, 311 and 312.

15. The antibody or antigen-binding fragment thereof of any one of the preceding claims, wherein the first binding moiety comprises:
i. a HCDR1 comprising an amino acid sequence selected from the group consisting of SEQ ID NOs: 31, 44, 50, 96, 97, 98, 132, 140, 182 and 307;
ii. a HCDR2 comprising an amino acid sequence selected from the group consisting of SEQ ID NOs: 32, 33, 34, 45, 51, 99, 100, 101, 102, 121, 123, 129, 143, 183 and 308; and
iii. a HCDR3 comprising an amino acid sequence selected from the group consisting of SEQ ID NOs: 35, 36, 37, 38, 39, 40, 46, 52, 103, 104, 105, 106, 127, 130, 133, 135, 137, 144, 184 and 309.

16. The antibody or antigen-binding fragment thereof of any one of the preceding claims, wherein the first binding moiety comprises:
i. a LCDR1 comprising an amino acid sequence selected from the group consisting of SEQ ID NOs: 41, 47, 53, 107, 108, 109, 110, 111, 112, 113, 114, 115, 116, 185 and 310;
ii. a LCDR2 comprising an amino acid sequence selected from the group consisting of SEQ ID NOs: 42, 48, 54, 117, 186 and 311; and
iii. a LCDR3 comprising an amino acid sequence selected from the group consisting of SEQ ID NOs: 43, 49, 55, 118, 142, 149, 187 and 312.

17. The antibody or antigen-binding fragment thereof of any one of the preceding claims, wherein the first binding moiety comprises:
i. a HCDR1 comprising an amino acid sequence as set forth in SEQ ID NO: 31, a HCDR2 comprising an amino acid sequence as set forth in SEQ ID NO: 34, and a HCDR3 comprising an amino acid sequence as set forth in SEQ ID NO: 40;
ii. a HCDR1 comprising an amino acid sequence as set forth in SEQ ID NO: 31, a HCDR2 comprising an amino acid sequence as set forth in SEQ ID NO: 32, and a HCDR3 comprising an amino acid sequence as set forth in SEQ ID NO: 35;
iii. a HCDR1 comprising an amino acid sequence as set forth in SEQ ID NO: 31, a HCDR2 comprising an amino acid sequence as set forth in SEQ ID NO: 32, and a HCDR3 comprising an amino acid sequence as set forth in SEQ ID NO: 36;
iv. a HCDR1 comprising an amino acid sequence as set forth in SEQ ID NO: 31, a HCDR2 comprising an amino acid sequence as set forth in SEQ ID NO: 32, and a HCDR3 comprising an amino acid sequence as set forth in SEQ ID NO: 37;
v. a HCDR1 comprising an amino acid sequence as set forth in SEQ ID NO: 31, a HCDR2 comprising an amino acid sequence as set forth in SEQ ID NO: 32, and a HCDR3 comprising an amino acid sequence as set forth in SEQ ID NO: 38;
vi. a HCDR1 comprising an amino acid sequence as set forth in SEQ ID NO: 31, a HCDR2 comprising an amino acid sequence as set forth in SEQ ID NO: 33, and a HCDR3 comprising an amino acid sequence as set forth in SEQ ID NO: 35;
vii. a HCDR1 comprising an amino acid sequence as set forth in SEQ ID NO: 31, a HCDR2 comprising an amino acid sequence as set forth in SEQ ID NO: 32, and a HCDR3 comprising an amino acid sequence as set forth in SEQ ID NO: 39;
viii. a HCDR1 comprising an amino acid sequence as set forth in SEQ ID NO: 44, a HCDR2 comprising an amino acid sequence as set forth in SEQ ID NO: 45, and a HCDR3 comprising an amino acid sequence as set forth in SEQ ID NO: 46;
ix. a HCDR1 comprising an amino acid sequence as set forth in SEQ ID NO: 50, a HCDR2 comprising an amino acid sequence as set forth in SEQ ID NO: 51, and a HCDR3 comprising an amino acid sequence as set forth in SEQ ID NO: 52;
x. a HCDR1 comprising an amino acid sequence as set forth in SEQ ID NO: 98, a HCDR2 comprising an amino acid sequence as set forth in SEQ ID NO: 102, and a HCDR3 comprising an amino acid sequence as set forth in SEQ ID NO: 106;
xi. a HCDR1 comprising an amino acid sequence as set forth in SEQ ID NO: 96, a HCDR2 comprising an amino acid sequence as set forth in SEQ ID NO: 99, and a HCDR3 comprising an amino acid sequence as set forth in SEQ ID NO: 103;
xii. a HCDR1 comprising an amino acid sequence as set forth in SEQ ID NO: 96, a HCDR2 comprising an amino acid sequence as set forth in SEQ ID NO: 100, and a HCDR3 comprising an amino acid sequence as set forth in SEQ ID NO: 103;
xiii. a HCDR1 comprising an amino acid sequence as set forth in SEQ ID NO: 97, a HCDR2 comprising an amino acid sequence as set forth in SEQ ID NO: 100, and a HCDR3 comprising an amino acid sequence as set forth in SEQ ID NO: 103;
xiv. a HCDR1 comprising an amino acid sequence as set forth in SEQ ID NO: 96, a HCDR2 comprising an amino acid sequence as set forth in SEQ ID NO: 101, and a HCDR3 comprising an amino acid sequence as set forth in SEQ ID NO: 103;
xv. a HCDR1 comprising an amino acid sequence as set forth in SEQ ID NO: 96, a HCDR2 comprising an amino acid sequence as set forth in SEQ ID NO: 100, and a HCDR3 comprising an amino acid sequence as set forth in SEQ ID NO: 104;
xvi. a HCDR1 comprising an amino acid sequence as set forth in SEQ ID NO: 96, a HCDR2 comprising an amino acid sequence as set forth in SEQ ID NO: 100, and a HCDR3 comprising an amino acid sequence as set forth in SEQ ID NO: 105;
xvii. a HCDR1 comprising an amino acid sequence as set forth in SEQ ID NO: 132, a HCDR2 comprising an amino acid sequence as set forth in SEQ ID NO: 143, and a HCDR3 comprising an amino acid sequence as set forth in SEQ ID NO: 144;
xviii. a HCDR1 comprising an amino acid sequence as set forth in SEQ ID NO: 31, a HCDR2 comprising an amino acid sequence as set forth in SEQ ID NO: 121, and a HCDR3 comprising an amino acid sequence as set forth in SEQ ID NO: 35;
xix. a HCDR1 comprising an amino acid sequence as set forth in SEQ ID NO: 31, a HCDR2 comprising an amino acid sequence as set forth in SEQ ID NO: 123, and a HCDR3 comprising an amino acid sequence as set forth in SEQ ID NO: 35;
XX. a HCDR1 comprising an amino acid sequence as set forth in SEQ ID NO: 31, a HCDR2 comprising an amino acid sequence as set forth in SEQ ID NO: 32, and a HCDR3 comprising an amino acid sequence as set forth in SEQ ID NO: 127;
xxi. a HCDR1 comprising an amino acid sequence as set forth in SEQ ID NO: 31, a HCDR2 comprising an amino acid sequence as set forth in SEQ ID NO: 129, and a HCDR3 comprising an amino acid sequence as set forth in SEQ ID NO: 130;
xxii. a HCDR1 comprising an amino acid sequence as set forth in SEQ ID NO: 31, a HCDR2 comprising an amino acid sequence as set forth in SEQ ID NO: 121, and a HCDR3 comprising an amino acid sequence as set forth in SEQ ID NO: 133;
xxiii. a HCDR1 comprising an amino acid sequence as set forth in SEQ ID NO: 31, a HCDR2 comprising an amino acid sequence as set forth in SEQ ID NO: 32, and a HCDR3 comprising an amino acid sequence as set forth in SEQ ID NO: 135;
xxiv. a HCDR1 comprising an amino acid sequence as set forth in SEQ ID NO: 31, a HCDR2 comprising an amino acid sequence as set forth in SEQ ID NO: 32, and a HCDR3 comprising an amino acid sequence as set forth in SEQ ID NO: 137;
xxv. a HCDR1 comprising an amino acid sequence as set forth in SEQ ID NO: 140, a HCDR2 comprising an amino acid sequence as set forth in SEQ ID NO: 32, and a HCDR3 comprising an amino acid sequence as set forth in SEQ ID NO: 35;
xxvi. a HCDR1 comprising an amino acid sequence as set forth in SEQ ID NO: 182, a HCDR2 comprising an amino acid sequence as set forth in SEQ ID NO: 183, and a HCDR3 comprising an amino acid sequence as set forth in SEQ ID NO: 184; or
xxvii. a HCDR1 comprising an amino acid sequence as set forth in SEQ ID NO: 307, a HCDR2 comprising an amino acid sequence as set forth in SEQ ID NO: 308, and a HCDR3 comprising an amino acid sequence as set forth in SEQ ID NO: 309.

18. The antibody or antigen-binding fragment thereof of any one of the preceding claims, wherein the first binding moiety comprises:
i. a LCDR1 comprising an amino acid sequence as set forth in SEQ ID NO: 41, a LCDR2 comprising an amino acid sequence as set forth in SEQ ID NO: 42, and a LCDR3 comprising an amino acid sequence as set forth in SEQ ID NO: 43;
ii. a LCDR1 comprising an amino acid sequence as set forth in SEQ ID NO: 47, a LCDR2 comprising an amino acid sequence as set forth in SEQ ID NO: 48, and a LCDR3 comprising an amino acid sequence as set forth in SEQ ID NO: 49;
iii. a LCDR1 comprising an amino acid sequence as set forth in SEQ ID NO: 53, a LCDR2 comprising an amino acid sequence as set forth in SEQ ID NO: 54, and a LCDR3 comprising an amino acid sequence as set forth in SEQ ID NO: 55;
iv. a LCDR1 comprising an amino acid sequence as set forth in SEQ ID NO: 116, a LCDR2 comprising an amino acid sequence as set forth in SEQ ID NO: 117, and a LCDR3 comprising an amino acid sequence as set forth in SEQ ID NO: 118;
v. a LCDR1 comprising an amino acid sequence as set forth in SEQ ID NO: 107, a LCDR2 comprising an amino acid sequence as set forth in SEQ ID NO: 117, and a LCDR3 comprising an amino acid sequence as set forth in SEQ ID NO: 118;
vi. a LCDR1 comprising an amino acid sequence as set forth in SEQ ID NO: 108, a LCDR2 comprising an amino acid sequence as set forth in SEQ ID NO: 117, and a LCDR3 comprising an amino acid sequence as set forth in SEQ ID NO: 118;
vii. a LCDR1 comprising an amino acid sequence as set forth in SEQ ID NO: 109, a LCDR2 comprising an amino acid sequence as set forth in SEQ ID NO: 117, and a LCDR3 comprising an amino acid sequence as set forth in SEQ ID NO: 118;
viii. a LCDR1 comprising an amino acid sequence as set forth in SEQ ID NO: 110, a LCDR2 comprising an amino acid sequence as set forth in SEQ ID NO: 117, and a LCDR3 comprising an amino acid sequence as set forth in SEQ ID NO: 118;
ix. a LCDR1 comprising an amino acid sequence as set forth in SEQ ID NO: 111, a LCDR2 comprising an amino acid sequence as set forth in SEQ ID NO: 117, and a LCDR3 comprising an amino acid sequence as set forth in SEQ ID NO: 118;
x. a LCDR1 comprising an amino acid sequence as set forth in SEQ ID NO: 112, a LCDR2 comprising an amino acid sequence as set forth in SEQ ID NO: 117, and a LCDR3 comprising an amino acid sequence as set forth in SEQ ID NO: 118;
xi. a LCDR1 comprising an amino acid sequence as set forth in SEQ ID NO: 113, a LCDR2 comprising an amino acid sequence as set forth in SEQ ID NO: 117, and a LCDR3 comprising an amino acid sequence as set forth in SEQ ID NO: 118;
xii. a LCDR1 comprising an amino acid sequence as set forth in SEQ ID NO: 114, a LCDR2 comprising an amino acid sequence as set forth in SEQ ID NO: 117, and a LCDR3 comprising an amino acid sequence as set forth in SEQ ID NO: 118;
xiii. a LCDR1 comprising an amino acid sequence as set forth in SEQ ID NO: 115, a LCDR2 comprising an amino acid sequence as set forth in SEQ ID NO: 117, and a LCDR3 comprising an amino acid sequence as set forth in SEQ ID NO: 118;
xiv. a LCDR1 comprising an amino acid sequence as set forth in SEQ ID NO: 41, a LCDR2 comprising an amino acid sequence as set forth in SEQ ID NO: 42, and a LCDR3 comprising an amino acid sequence as set forth in SEQ ID NO: 142;
xv. a LCDR1 comprising an amino acid sequence as set forth in SEQ ID NO: 41, a LCDR2 comprising an amino acid sequence as set forth in SEQ ID NO: 42, and a LCDR3 comprising an amino acid sequence as set forth in SEQ ID NO: 149;
xvi. a LCDR1 comprising an amino acid sequence as set forth in SEQ ID NO: 185, a LCDR2 comprising an amino acid sequence as set forth in SEQ ID NO: 186, and a LCDR3 comprising an amino acid sequence as set forth in SEQ ID NO: 187; or
xvii. a LCDR1 comprising an amino acid sequence as set forth in SEQ ID NO: 310, a LCDR2 comprising an amino acid sequence as set forth in SEQ ID NO: 311, and a LCDR3 comprising an amino acid sequence as set forth in SEQ ID NO: 312.

19. The antibody or antigen-binding fragment thereof of any one of the preceding claims, wherein the first binding moiety comprises:
i. a HCDR1 comprising an amino acid sequence as set forth in SEQ ID NO: 31, a HCDR2 comprising an amino acid sequence as set forth in SEQ ID NO: 34, and a HCDR3 comprising an amino acid sequence as set forth in SEQ ID NO: 40, a LCDR1 comprising an amino acid sequence as set forth in SEQ ID NO: 41, a LCDR2 comprising an amino acid sequence as set forth in SEQ ID NO: 42, and a LCDR3 comprising an amino acid sequence as set forth in SEQ ID NO: 43;
ii. a HCDR1 comprising an amino acid sequence as set forth in SEQ ID NO: 31, a HCDR2 comprising an amino acid sequence as set forth in SEQ ID NO: 32, and a HCDR3 comprising an amino acid sequence as set forth in SEQ ID NO: 35, a LCDR1 comprising an amino acid sequence as set forth in SEQ ID NO: 41, a LCDR2 comprising an amino acid sequence as set forth in SEQ ID NO: 42, and a LCDR3 comprising an amino acid sequence as set forth in SEQ ID NO: 43;
iii. a HCDR1 comprising an amino acid sequence as set forth in SEQ ID NO: 31, a HCDR2 comprising an amino acid sequence as set forth in SEQ ID NO: 32, and a HCDR3 comprising an amino acid sequence as set forth in SEQ ID NO: 36, a LCDR1 comprising an amino acid sequence as set forth in SEQ ID NO: 41, a LCDR2 comprising an amino acid sequence as set forth in SEQ ID NO: 42, and a LCDR3 comprising an amino acid sequence as set forth in SEQ ID NO: 43;
iv. a HCDR1 comprising an amino acid sequence as set forth in SEQ ID NO: 31, a HCDR2 comprising an amino acid sequence as set forth in SEQ ID NO: 32, and a HCDR3 comprising an amino acid sequence as set forth in SEQ ID NO: 37, a LCDR1 comprising an amino acid sequence as set forth in SEQ ID NO: 41, a LCDR2 comprising an amino acid sequence as set forth in SEQ ID NO: 42, and a LCDR3 comprising an amino acid sequence as set forth in SEQ ID NO: 43;
v. a HCDR1 comprising an amino acid sequence as set forth in SEQ ID NO: 31, a HCDR2 comprising an amino acid sequence as set forth in SEQ ID NO: 32, and a HCDR3 comprising an amino acid sequence as set forth in SEQ ID NO: 38, a LCDR1 comprising an amino acid sequence as set forth in SEQ ID NO: 41, a LCDR2 comprising an amino acid sequence as set forth in SEQ ID NO: 42, and a LCDR3 comprising an amino acid sequence as set forth in SEQ ID NO: 43;
vi. a HCDR1 comprising an amino acid sequence as set forth in SEQ ID NO: 31, a HCDR2 comprising an amino acid sequence as set forth in SEQ ID NO: 33, and a HCDR3 comprising an amino acid sequence as set forth in SEQ ID NO: 35, a LCDR1 comprising an amino acid sequence as set forth in SEQ ID NO: 41, a LCDR2 comprising an amino acid sequence as set forth in SEQ ID NO: 42, and a LCDR3 comprising an amino acid sequence as set forth in SEQ ID NO: 43;
vii. a HCDR1 comprising an amino acid sequence as set forth in SEQ ID NO: 31, a HCDR2 comprising an amino acid sequence as set forth in SEQ ID NO: 32, and a HCDR3 comprising an amino acid sequence as set forth in SEQ ID NO: 39, a LCDR1 comprising an amino acid sequence as set forth in SEQ ID NO: 41, a LCDR2 comprising an amino acid sequence as set forth in SEQ ID NO: 42, and a LCDR3 comprising an amino acid sequence as set forth in SEQ ID NO: 43;
viii. a HCDR1 comprising an amino acid sequence as set forth in SEQ ID NO: 44, a HCDR2 comprising an amino acid sequence as set forth in SEQ ID NO: 45, and a HCDR3 comprising an amino acid sequence as set forth in SEQ ID NO: 46, a LCDR1 comprising an amino acid sequence as set forth in SEQ ID NO: 47, a LCDR2 comprising an amino acid sequence as set forth in SEQ ID NO: 48, and a LCDR3 comprising an amino acid sequence as set forth in SEQ ID NO: 49;
ix. a HCDR1 comprising an amino acid sequence as set forth in SEQ ID NO: 50, a HCDR2 comprising an amino acid sequence as set forth in SEQ ID NO: 51, and a HCDR3 comprising an amino acid sequence as set forth in SEQ ID NO: 52, a LCDR1 comprising an amino acid sequence as set forth in SEQ ID NO: 53, a LCDR2 comprising an amino acid sequence as set forth in SEQ ID NO: 54, and a LCDR3 comprising an amino acid sequence as set forth in SEQ ID NO: 55;
x. a HCDR1 comprising an amino acid sequence as set forth in SEQ ID NO: 98, a HCDR2 comprising an amino acid sequence as set forth in SEQ ID NO: 102, and a HCDR3 comprising an amino acid sequence as set forth in SEQ ID NO: 106, a LCDR1 comprising an amino acid sequence as set forth in SEQ ID NO: 116, a LCDR2 comprising an amino acid sequence as set forth in SEQ ID NO: 117, and a LCDR3 comprising an amino acid sequence as set forth in SEQ ID NO: 118;
xi. a HCDR1 comprising an amino acid sequence as set forth in SEQ ID NO: 96, a HCDR2 comprising an amino acid sequence as set forth in SEQ ID NO: 99, and a HCDR3 comprising an amino acid sequence as set forth in SEQ ID NO: 103, a LCDR1 comprising an amino acid sequence as set forth in SEQ ID NO: 107, a LCDR2 comprising an amino acid sequence as set forth in SEQ ID NO: 117, and a LCDR3 comprising an amino acid sequence as set forth in SEQ ID NO: 118;
xii. a HCDR1 comprising an amino acid sequence as set forth in SEQ ID NO: 97, a HCDR2 comprising an amino acid sequence as set forth in SEQ ID NO: 100, and a HCDR3 comprising an amino acid sequence as set forth in SEQ ID NO: 103, a LCDR1 comprising an amino acid sequence as set forth in SEQ ID NO: 107, a LCDR2 comprising an amino acid sequence as set forth in SEQ ID NO: 117, and a LCDR3 comprising an amino acid sequence as set forth in SEQ ID NO: 118;
xiii. a HCDR1 comprising an amino acid sequence as set forth in SEQ ID NO: 96, a HCDR2 comprising an amino acid sequence as set forth in SEQ ID NO: 101, and a HCDR3 comprising an amino acid sequence as set forth in SEQ ID NO: 103, a LCDR1 comprising an amino acid sequence as set forth in SEQ ID NO: 107, a LCDR2 comprising an amino acid sequence as set forth in SEQ ID NO: 117, and a LCDR3 comprising an amino acid sequence as set forth in SEQ ID NO: 118;
xiv. a HCDR1 comprising an amino acid sequence as set forth in SEQ ID NO: 96, a HCDR2 comprising an amino acid sequence as set forth in SEQ ID NO: 100, and a HCDR3 comprising an amino acid sequence as set forth in SEQ ID NO: 104, a LCDR1 comprising an amino acid sequence as set forth in SEQ ID NO: 107, a LCDR2 comprising an amino acid sequence as set forth in SEQ ID NO: 117, and a LCDR3 comprising an amino acid sequence as set forth in SEQ ID NO: 118;
xv. a HCDR1 comprising an amino acid sequence as set forth in SEQ ID NO: 96, a HCDR2 comprising an amino acid sequence as set forth in SEQ ID NO: 100, and a HCDR3 comprising an amino acid sequence as set forth in SEQ ID NO: 105, a LCDR1 comprising an amino acid sequence as set forth in SEQ ID NO: 107, a LCDR2 comprising an amino acid sequence as set forth in SEQ ID NO: 117, and a LCDR3 comprising an amino acid sequence as set forth in SEQ ID NO: 118;
xvi. a HCDR1 comprising an amino acid sequence as set forth in SEQ ID NO: 96, a HCDR2 comprising an amino acid sequence as set forth in SEQ ID NO: 100, and a HCDR3 comprising an amino acid sequence as set forth in SEQ ID NO: 103, a LCDR1 comprising an amino acid sequence as set forth in SEQ ID NO: 108, a LCDR2 comprising an amino acid sequence as set forth in SEQ ID NO: 117, and a LCDR3 comprising an amino acid sequence as set forth in SEQ ID NO: 118;
xvii. a HCDR1 comprising an amino acid sequence as set forth in SEQ ID NO: 96, a HCDR2 comprising an amino acid sequence as set forth in SEQ ID NO: 100, and a HCDR3 comprising an amino acid sequence as set forth in SEQ ID NO: 103, a LCDR1 comprising an amino acid sequence as set forth in SEQ ID NO: 109, a LCDR2 comprising an amino acid sequence as set forth in SEQ ID NO: 117, and a LCDR3 comprising an amino acid sequence as set forth in SEQ ID NO: 118;
xviii. a HCDR1 comprising an amino acid sequence as set forth in SEQ ID NO: 96, a HCDR2 comprising an amino acid sequence as set forth in SEQ ID NO: 100, and a HCDR3 comprising an amino acid sequence as set forth in SEQ ID NO: 103, a LCDR1 comprising an amino acid sequence as set forth in SEQ ID NO: 110, a LCDR2 comprising an amino acid sequence as set forth in SEQ ID NO: 117, and a LCDR3 comprising an amino acid sequence as set forth in SEQ ID NO: 118;
xix. a HCDR1 comprising an amino acid sequence as set forth in SEQ ID NO: 96, a HCDR2 comprising an amino acid sequence as set forth in SEQ ID NO: 100, and a HCDR3 comprising an amino acid sequence as set forth in SEQ ID NO: 103, a LCDR1 comprising an amino acid sequence as set forth in SEQ ID NO: 111, a LCDR2 comprising an amino acid sequence as set forth in SEQ ID NO: 117, and a LCDR3 comprising an amino acid sequence as set forth in SEQ ID NO: 118;
XX. a HCDR1 comprising an amino acid sequence as set forth in SEQ ID NO: 96, a HCDR2 comprising an amino acid sequence as set forth in SEQ ID NO: 100, and a HCDR3 comprising an amino acid sequence as set forth in SEQ ID NO: 103, a LCDR1 comprising an amino acid sequence as set forth in SEQ ID NO: 112, a LCDR2 comprising an amino acid sequence as set forth in SEQ ID NO: 117, and a LCDR3 comprising an amino acid sequence as set forth in SEQ ID NO: 118;
xxi. a HCDR1 comprising an amino acid sequence as set forth in SEQ ID NO: 96, a HCDR2 comprising an amino acid sequence as set forth in SEQ ID NO: 100, and a HCDR3 comprising an amino acid sequence as set forth in SEQ ID NO: 103, a LCDR1 comprising an amino acid sequence as set forth in SEQ ID NO: 113, a LCDR2 comprising an amino acid sequence as set forth in SEQ ID NO: 117, and a LCDR3 comprising an amino acid sequence as set forth in SEQ ID NO: 118;
xxii. a HCDR1 comprising an amino acid sequence as set forth in SEQ ID NO: 96, a HCDR2 comprising an amino acid sequence as set forth in SEQ ID NO: 100, and a HCDR3 comprising an amino acid sequence as set forth in SEQ ID NO: 103, a LCDR1 comprising an amino acid sequence as set forth in SEQ ID NO: 114, a LCDR2 comprising an amino acid sequence as set forth in SEQ ID NO: 117, and a LCDR3 comprising an amino acid sequence as set forth in SEQ ID NO: 118;
xxiii. a HCDR1 comprising an amino acid sequence as set forth in SEQ ID NO: 96, a HCDR2 comprising an amino acid sequence as set forth in SEQ ID NO: 100, and a HCDR3 comprising an amino acid sequence as set forth in SEQ ID NO: 103, a LCDR1 comprising an amino acid sequence as set forth in SEQ ID NO: 115, a LCDR2 comprising an amino acid sequence as set forth in SEQ ID NO: 117, and a LCDR3 comprising an amino acid sequence sequence as set forth in SEQ ID NO: 118;
xxiv. a HCDR1 comprising an amino acid sequence as set forth in SEQ ID NO: 31, a HCDR2 comprising an amino acid sequence as set forth in SEQ ID NO: 121, and a HCDR3 comprising an amino acid sequence as set forth in SEQ ID NO: 35, a LCDR1 comprising an amino acid sequence as set forth in SEQ ID NO: 41, a LCDR2 comprising an amino acid sequence as set forth in SEQ ID NO: 42, and a LCDR3 comprising an amino acid sequence as set forth in SEQ ID NO: 43;
xxv. a HCDR1 comprising an amino acid sequence as set forth in SEQ ID NO: 31, a HCDR2 comprising an amino acid sequence as set forth in SEQ ID NO: 123, and a HCDR3 comprising an amino acid sequence as set forth in SEQ ID NO: 35, a LCDR1 comprising an amino acid sequence as set forth in SEQ ID NO: 41, a LCDR2 comprising an amino acid sequence as set forth in SEQ ID NO: 42, and a LCDR3 comprising an amino acid sequence as set forth in SEQ ID NO: 43;
xxvi. a HCDR1 comprising an amino acid sequence as set forth in SEQ ID NO: 31, a HCDR2 comprising an amino acid sequence as set forth in SEQ ID NO: 32, and a HCDR3 comprising an amino acid sequence as set forth in SEQ ID NO: 127, a LCDR1 comprising an amino acid sequence as set forth in SEQ ID NO: 41, a LCDR2 comprising an amino acid sequence as set forth in SEQ ID NO: 42, and a LCDR3 comprising an amino acid sequence as set forth in SEQ ID NO: 43;
xxvii. a HCDR1 comprising an amino acid sequence as set forth in SEQ ID NO: 31, a HCDR2 comprising an amino acid sequence as set forth in SEQ ID NO: 129, and a HCDR3 comprising an amino acid sequence as set forth in SEQ ID NO: 130, a LCDR1 comprising an amino acid sequence as set forth in SEQ ID NO: 41, a LCDR2 comprising an amino acid sequence as set forth in SEQ ID NO: 42, and a LCDR3 comprising an amino acid sequence as set forth in SEQ ID NO: 43;
xxviii. a HCDR1 comprising an amino acid sequence as set forth in SEQ ID NO: 31, a HCDR2 comprising an amino acid sequence as set forth in SEQ ID NO: 121, and a HCDR3 comprising an amino acid sequence as set forth in SEQ ID NO: 133, a LCDR1 comprising an amino acid sequence as set forth in SEQ ID NO: 41, a LCDR2 comprising an amino acid sequence as set forth in SEQ ID NO: 42, and a LCDR3 comprising an amino acid sequence as set forth in SEQ ID NO: 43;
xxix. a HCDR1 comprising an amino acid sequence as set forth in SEQ ID NO: 31, a HCDR2 comprising an amino acid sequence as set forth in SEQ ID NO: 32, and a HCDR3 comprising an amino acid sequence as set forth in SEQ ID NO: 135, a LCDR1 comprising an amino acid sequence as set forth in SEQ ID NO: 41, a LCDR2 comprising an amino acid sequence as set forth in SEQ ID NO: 42, and a LCDR3 comprising an amino acid sequence as set forth in SEQ ID NO: 43;
xxx. a HCDR1 comprising an amino acid sequence as set forth in SEQ ID NO: 31, a HCDR2 comprising an amino acid sequence as set forth in SEQ ID NO: 32, and a HCDR3 comprising an amino acid sequence as set forth in SEQ ID NO: 137, a LCDR1 comprising an amino acid sequence as set forth in SEQ ID NO: 41, a LCDR2 comprising an amino acid sequence as set forth in SEQ ID NO: 42, and a LCDR3 comprising an amino acid sequence as set forth in SEQ ID NO: 43;
xxxi. a HCDR1 comprising an amino acid sequence as set forth in SEQ ID NO: 140, a HCDR2 comprising an amino acid sequence as set forth in SEQ ID NO: 32, and a HCDR3 comprising an amino acid sequence as set forth in SEQ ID NO: 35, a LCDR1 comprising an amino acid sequence as set forth in SEQ ID NO: 41, a LCDR2 comprising an amino acid sequence as set forth in SEQ ID NO: 42, and a LCDR3 comprising an amino acid sequence as set forth in SEQ ID NO: 43; or
xxxii. a HCDR1 comprising an amino acid sequence as set forth in SEQ ID NO: 132, a HCDR2 comprising an amino acid sequence as set forth in SEQ ID NO: 143, and a HCDR3 comprising an amino acid sequence as set forth in SEQ ID NO: 144, a LCDR1 comprising an amino acid sequence as set forth in SEQ ID NO: 41, a LCDR2 comprising an amino acid sequence as set forth in SEQ ID NO: 42, and a LCDR3 comprising an amino acid sequence as set forth in SEQ ID NO: 149;
xxxiii. a HCDR1 comprising an amino acid sequence as set forth in SEQ ID NO: 31, a HCDR2 comprising an amino acid sequence as set forth in SEQ ID NO: 129, and a HCDR3 comprising an amino acid sequence as set forth in SEQ ID NO: 130, a LCDR1 comprising an amino acid sequence as set forth in SEQ ID NO: 41, a LCDR2 comprising an amino acid sequence as set forth in SEQ ID NO: 42, and a LCDR3 comprising an amino acid sequence as set forth in SEQ ID NO: 142; or
xxxiv. a HCDR1 comprising an amino acid sequence as set forth in SEQ ID NO: 307, a HCDR2 comprising an amino acid sequence as set forth in SEQ ID NO: 308, and a HCDR3 comprising an amino acid sequence as set forth in SEQ ID NO: 309, a LCDR1 comprising an amino acid sequence as set forth in SEQ ID NO: 310, a LCDR2 comprising an amino acid sequence as set forth in SEQ ID NO: 311, and a LCDR3 comprising an amino acid sequence as set forth in SEQ ID NO: 312.

20. The antibody or antigen-binding fragment thereof of any one of the preceding claims, wherein the first binding moiety comprises a VH region having an amino acid sequence as set forth in SEQ ID NOs: 3, 7, 11, 21, 22, 23, 26, 27, 28, 29, 30, 80, 82, 83, 84, 85, 86, 119, 120, 122, 124, 125, 126, 128, 131, 134, 136, 138, 139, 145, 195, 196, 197, 200, 201, 202, 203, 204, 205, 207, 208, 209, 210, 211, 221, 222, 223, 224, 225, 226, 227, 228, 229, 230, 231, 232 or 315, or a homologous sequence thereof having at least 80% sequence identity to SEQ ID NOs: 3, 7, 11, 21, 22, 23, 26, 27, 28, 29, 30, 80, 82, 83, 84, 85, 86, 119, 120, 122, 124, 125, 126, 128, 131, 134, 136, 138, 139, 145, 195, 196, 197, 200, 201, 202, 203, 204, 205, 207, 208, 209, 210, 211, 221, 222, 223, 224, 225, 226, 227, 228, 229, 230, 231, 232 or 315.

21. The antibody or antigen-binding fragment thereof of any one of the preceding claims, wherein the first binding moiety comprises a VL region having an amino acid sequence as set forth in SEQ ID NOs: 4, 8, 12, 24, 25, 81, 87, 88, 89, 90, 91, 92, 93, 94, 95, 141, 146, 198, 199, 206, 212, 213, 214, 215, 216, 217, 218, 219, 220, 233, 234 or 316, or a homologous sequence thereof having at least 80% sequence identity to SEQ ID NOs: 4, 8, 12, 24, 25, 81, 87, 88, 89, 90, 91, 92, 93, 94, 95, 141, 146, 198, 199, 206, 212, 213, 214, 215, 216, 217, 218, 219, 220, 233, 234 or 316.

22. The antibody or antigen-binding fragment thereof of any one of the preceding claims, wherein the first binding moiety comprises a VH/VL amino acid sequence pair selected from the group consisting of SEQ ID NOs: 3/4, 7/8, 11/12, 21/24, 22/24, 23/24, 21/25, 26/24, 27/24, 28/24, 29/24, 30/24, 80/81, 83/87, 84/87, 85/87, 86/87, 82/88, 82/89, 82/90, 82/91, 82/92, 82/93, 82/94, 82/95, 119/198, 120/198, 122/198, 124/198, 125/198, 126/198, 128/198, 131/198, 134/198, 136/198, 138/198, 139/198, 128/141, 195/198, 196/198, 197/198, 195/199, 200/198, 201/198, 202/198, 203/198, 204/198, 205/206, 208/212, 209/212, 210/212, 211/212, 207/213, 207/214, 207/215, 207/216, 207/217, 207/218, 207/219, 207/220, 221/233, 222/233, 223/233, 224/233, 225/233, 226/233, 227/233, 227/234, 228/233, 229/233, 230/233, 231/233, 232/233 and 315/316.

23. The antibody or antigen-binding fragment thereof of any one of the preceding claims, wherein the first binding moiety further comprises one or more amino acid residue substitutions or modifications yet retains specific binding affinity to CD3.

24. The antibody or antigen-binding fragment thereof of claim 23, wherein at least one of the substitutions or modifications is within one or more of the CDR sequences of the VH region or VL region of the first binding moiety.

25. The antibody or antigen-binding fragment thereof of claim 23, wherein at least one of the substitutions or modifications is within one or more of the non-CDR sequences of the VH region or VL region of the first binding moiety.

26. The antibody or antigen-binding fragment thereof of the proceeding claims, further comprising one or more non-natural amino acid (NNAA) substitution.

27. The antibody or antigen-binding fragment thereof of claim 26, wherein the NNAA is capable of being conjugated.

28. The antibody or antigen-binding fragment thereof of any one of the preceding claims, which is a chimeric, a humanized or a human antibody or an antigen-binding fragment thereof.

29. The antibody or antigen-binding fragment thereof of any one of the preceding claims, which is a labeled antibody, a bivalent antibody, an anti-idiotypic antibody or a fusion protein.

30. The antibody or antigen-binding fragment thereof of any one of the preceding claims, further comprising an Fc region, optionally an Fc region of human immunoglobulin (Ig), or optionally an Fc region of human IgG.

31. The antibody or antigen-binding fragment thereof of claim 30, wherein the Fc region is derived from human IgG1, IgG2, IgG3, or IgG4.

32. The antibody or antigen-binding fragment thereof of claim 31, wherein both of the first binding moiety and the third binding moiety are linked to the Fc region.

33. The antibody or antigen-binding fragment thereof of any one of the preceding claims, wherein the light chain is a 2 light chain or a k light chain.

34. The antibody or antigen-binding fragment thereof of any one of the preceding claims, wherein the second target is selected from the group consisting of GPRC5D, GD2, CD16a, CD33, CD3ε, CD45, CD123, CD146, CD228, CLL-1, FLT3, FLT3L, TAF1, TgPRF, HVCN1, IL-6R, IL-11R, IL17A, IL-23R, IL-33, ILDR2, LAP, TSLP, TREM-1, ANGPT2, APOE, IFNAR, CypA, DOG-1, NKp30, CSF-1R, CCR2, LRRC15, mesothelin, Dickkopf2, DLL3, HER-2, C10orf54, TrkA, MEKK1, KRAS, ERK, XPO1, mTORC1/2, PAK4, NAMPT, ATR, EGFR, FGFR, VEGF, LILRB (e.g., LILRB1, LILRB2, LILRB3, LILRB4, LILRB5), c-MET, Her2, Her3, CTLA4, GITA, CD112R, CD2, CD7, CD16, CD19, CD20, CD24, CD27, CD30, CD34, CD37, CD39, CD70, CD73, CD83, CD28, CD80 (B7-1), CD86 (B7-2), CD40, CD40L (CD154), CD47, SIRPa, CD122, CD137, CD137L, OX40 (CD134), OX40L (CD252), BCMA (e.g., BCMA02), PSMA, CLDN18 (e.g., CLDN18.2), NKG2C, 4-1BB, LIGHT, PVRIG, SLAMF7, HVEM, BAFFR, ICAM-1, 2B4, LFA-1, GITR, ICOS (CD278), ICOSLG (CD275), LAG3 (CD223), A2AR, B7-H3 (CD276), B7-H4 (VTCN1), B7-H5, BTLA (CD272), CD160, CTLA-4 (CD152), IDO (e.g., IDO1, IDO2), ILT3, TDO, KIR, LAIR-1, NOX2, PD-1, PD-L1, PD-L2, TIM-3, VISTA, SIGLEC-7 (CD328), SIGLEC-9 (CD329), SIGLEC-15, TIGIT, PVR (CD155), TLR3, CLEC9A, DEC-205, STING, and TGFB.

35. The antibody or antigen-binding fragment thereof of claim 34, wherein the second target is GPRC5D, GD2, LILRB4, CD19 or CD20.

36. The antibody or antigen-binding fragment thereof of claim 35, wherein the second binding moiety comprises
one, two or three heavy chain complementarity determining regions (HCDR1, HCDR2 and/or HCDR3) contained within any one of the heavy chain variable (VH) region sequences selected from the group consisting of SEQ ID NOs: 1, 5, 9, 13, 14, 15, 16, 19, 147, 243, 257, 270, 282, 294, 313 and 326; and/or
one, two or three light chain complementarity determining regions (LCDR1, LCDR2 and/or LCDR3) contained within any one of the light chain variable (VL) region sequences selected from the group consisting of SEQ ID NOs: 2, 6, 10, 17, 18, 20, 148, 247, 258, 271, 283, 295, 314 and 327.

37. The antibody or antigen-binding fragment thereof of claim 36, wherein the second binding moiety comprises at least one heavy or light chain complementarity determining region (CDR) comprising an amino acid sequence selected from the group consisting of SEQ ID NOs: 56, 57, 58, 59, 60, 61, 62, 63, 64, 65, 66, 67, 68, 69, 70, 71, 72, 73, 74, 75, 76, 77, 78, 79, 188, 189, 190, 191, 192, 193, 251, 252, 253, 254, 255, 256, 264, 265, 266, 267, 268, 269, 276, 277, 278, 279, 280, 281, 288, 289, 290, 291, 292, 293, 301, 302, 303, 304, 305, 306, 320, 321, 322, 323, 324 and 325.

38. The antibody or antigen-binding fragment thereof of claim 36 or 37, wherein the second binding moiety comprises one or two or three of HCDR1, HCDR2 and HCDR3 comprising an amino acid sequence selected from the group consisting of SEQ ID NOs: 56, 57, 58, 62, 63, 64, 68, 69, 70, 74, 75, 76, 188, 189, 190, 251, 252, 253, 264, 265, 266, 276, 277, 278, 288, 289, 290, 301, 302, 303, 320, 321 and 322.

39. The antibody or antigen-binding fragment thereof of any one of claims 36 to 38, wherein the second binding moiety comprises one or two or three of LCDR1, LCDR2 and LCDR3 comprising an amino acid sequence selected from the group consisting of SEQ ID NOs: 59, 60, 61, 65, 66, 67, 71, 72, 73, 77, 78, 79, 191, 192, 193, 254, 255, 256, 267, 268, 269, 279, 280, 281, 291, 292, 293, 304, 305, 306, 323, 324 and 325.

40. The antibody or antigen-binding fragment thereof of any one of claims 36 to 39, wherein the second binding moiety comprises:
i. a HCDR1 comprising an amino acid sequence selected from the group consisting of SEQ ID NOs: 56, 62, 68, 74, 188, 251, 264, 276, 288, 301 and 320;
ii. a HCDR2 comprising an amino acid sequence selected from the group consisting of SEQ ID NOs: 57, 63, 69, 75, 189, 252, 265, 277, 289, 302 and 321; and
iii. a HCDR3 comprising an amino acid sequence selected from the group consisting of SEQ ID NOs: 58, 64, 70, 76, 190, 253, 266, 278, 290, 303 and 322.

41. The antibody or antigen-binding fragment thereof of any one of claims 36 to 40, wherein the second binding moiety comprises:
i. a LCDR1 comprising an amino acid sequence selected from the group consisting of SEQ ID NOs: 59, 65, 71, 77, 191, 254, 267, 279, 291, 304, 323;
ii. a LCDR2 comprising an amino acid sequence selected from the group consisting of SEQ ID NOs: 60, 66, 72, 78, 192, 255, 268, 280, 292, 305 and 324; and
iii. a LCDR3 comprising an amino acid sequence selected from the group consisting of SEQ ID NOs: 61, 67, 73, 79, 193, 256, 269, 281, 293, 306, 325.

42. The antibody or antigen-binding fragment thereof of any one of claims 36 to 41, wherein the second binding moiety comprises:
i. a HCDR1 comprising an amino acid sequence as set forth in SEQ ID NO: 56, a HCDR2 comprising an amino acid sequence as set forth in SEQ ID NO: 57, and a HCDR3 comprising an amino acid sequence as set forth in SEQ ID NO: 58;
ii. a HCDR1 comprising an amino acid sequence as set forth in SEQ ID NO: 62, a HCDR2 comprising an amino acid sequence as set forth in SEQ ID NO: 63, and a HCDR3 comprising an amino acid sequence as set forth in SEQ ID NO: 64;
iii. a HCDR1 comprising an amino acid sequence as set forth in SEQ ID NO: 68, a HCDR2 comprising an amino acid sequence as set forth in SEQ ID NO: 69, and a HCDR3 comprising an amino acid sequence as set forth in SEQ ID NO: 70;
iv. a HCDR1 comprising an amino acid sequence as set forth in SEQ ID NO: 74, a HCDR2 comprising an amino acid sequence as set forth in SEQ ID NO: 75, and a HCDR3 comprising an amino acid sequence as set forth in SEQ ID NO: 76;
v. a HCDR1 comprising an amino acid sequence as set forth in SEQ ID NO: 188, a HCDR2 comprising an amino acid sequence as set forth in SEQ ID NO: 189, and a HCDR3 comprising an amino acid sequence as set forth in SEQ ID NO: 190;
vi. a HCDR1 comprising an amino acid sequence as set forth in SEQ ID NO: 251, a HCDR2 comprising an amino acid sequence as set forth in SEQ ID NO: 252, and a HCDR3 comprising an amino acid sequence as set forth in SEQ ID NO: 253;
vii. a HCDR1 comprising an amino acid sequence as set forth in SEQ ID NO: 264, a HCDR2 comprising an amino acid sequence as set forth in SEQ ID NO: 265, and a HCDR3 comprising an amino acid sequence as set forth in SEQ ID NO: 266;
viii. a HCDR1 comprising an amino acid sequence as set forth in SEQ ID NO: 276, a HCDR2 comprising an amino acid sequence as set forth in SEQ ID NO: 277, and a HCDR3 comprising an amino acid sequence as set forth in SEQ ID NO: 278;
ix. a HCDR1 comprising an amino acid sequence as set forth in SEQ ID NO: 288, a HCDR2 comprising an amino acid sequence as set forth in SEQ ID NO: 289, and a HCDR3 comprising an amino acid sequence as set forth in SEQ ID NO: 290;
x. a HCDR1 comprising an amino acid sequence as set forth in SEQ ID NO: 301, a HCDR2 comprising an amino acid sequence as set forth in SEQ ID NO: 302, and a HCDR3 comprising an amino acid sequence as set forth in SEQ ID NO: 303; or
xi. a HCDR1 comprising an amino acid sequence as set forth in SEQ ID NO: 320, a HCDR2 comprising an amino acid sequence as set forth in SEQ ID NO: 321, and a HCDR3 comprising an amino acid sequence as set forth in SEQ ID NO: 322.

43. The antibody or antigen-binding fragment thereof of any one of claims 36 to 42, wherein the second binding moiety comprises:
i. a LCDR1 comprising an amino acid sequence as set forth in SEQ ID NO: 59, a LCDR2 comprising an amino acid sequence as set forth in SEQ ID NO: 60, and a LCDR3 comprising an amino acid sequence as set forth in SEQ ID NO: 61;
ii. a LCDR1 comprising an amino acid sequence as set forth in SEQ ID NO: 65, a LCDR2 comprising an amino acid sequence as set forth in SEQ ID NO: 66, and a LCDR3 comprising an amino acid sequence as set forth in SEQ ID NO: 67;
iii. a LCDR1 comprising an amino acid sequence as set forth in SEQ ID NO: 71, a LCDR2 comprising an amino acid sequence as set forth in SEQ ID NO: 72, and a LCDR3 comprising an amino acid sequence as set forth in SEQ ID NO: 73;
iv. a LCDR1 comprising an amino acid sequence as set forth in SEQ ID NO: 77, a LCDR2 comprising an amino acid sequence as set forth in SEQ ID NO: 78, and a LCDR3 comprising an amino acid sequence as set forth in SEQ ID NO: 79;
v. a LCDR1 comprising an amino acid sequence as set forth in SEQ ID NO: 191, a LCDR2 comprising an amino acid sequence as set forth in SEQ ID NO: 192, and a LCDR3 comprising an amino acid sequence as set forth in SEQ ID NO: 193;
vi. a LCDR1 comprising an amino acid sequence as set forth in SEQ ID NO: 254, a LCDR2 comprising an amino acid sequence as set forth in SEQ ID NO: 255, and a LCDR3 comprising an amino acid sequence as set forth in SEQ ID NO: 256;
vii. a LCDR1 comprising an amino acid sequence as set forth in SEQ ID NO: 267, a LCDR2 comprising an amino acid sequence as set forth in SEQ ID NO: 268, and a LCDR3 comprising an amino acid sequence as set forth in SEQ ID NO: 269;
viii. a LCDR1 comprising an amino acid sequence as set forth in SEQ ID NO: 279, a LCDR2 comprising an amino acid sequence as set forth in SEQ ID NO: 280, and a LCDR3 comprising an amino acid sequence as set forth in SEQ ID NO: 281;
ix. a LCDR1 comprising an amino acid sequence as set forth in SEQ ID NO: 291, a LCDR2 comprising an amino acid sequence as set forth in SEQ ID NO: 292, and a LCDR3 comprising an amino acid sequence as set forth in SEQ ID NO: 293;
x. a LCDR1 comprising an amino acid sequence as set forth in SEQ ID NO: 304, a LCDR2 comprising an amino acid sequence as set forth in SEQ ID NO: 305, and a LCDR3 comprising an amino acid sequence as set forth in SEQ ID NO: 306; or
xi. a LCDR1 comprising an amino acid sequence as set forth in SEQ ID NO: 323, a LCDR2 comprising an amino acid sequence as set forth in SEQ ID NO: 324, and a LCDR3 comprising an amino acid sequence as set forth in SEQ ID NO: 325.

44. The antibody or antigen-binding fragment thereof of any one of claims 36 to 43, wherein the second binding moiety comprises:
i. a HCDR1 comprising an amino acid sequence as set forth in SEQ ID NO: 56, a HCDR2 comprising an amino acid sequence as set forth in SEQ ID NO: 57, and a HCDR3 comprising an amino acid sequence as set forth in SEQ ID NO: 58, a LCDR1 comprising an amino acid sequence as set forth in SEQ ID NO: 59, a LCDR2 comprising an amino acid sequence as set forth in SEQ ID NO: 60, and a LCDR3 comprising an amino acid sequence as set forth in SEQ ID NO: 61;
ii. a HCDR1 comprising an amino acid sequence as set forth in SEQ ID NO: 62, a HCDR2 comprising an amino acid sequence as set forth in SEQ ID NO: 63, and a HCDR3 comprising an amino acid sequence as set forth in SEQ ID NO: 64; a LCDR1 comprising an amino acid sequence as set forth in SEQ ID NO: 65, a LCDR2 comprising an amino acid sequence as set forth in SEQ ID NO: 66, and a LCDR3 comprising an amino acid sequence as set forth in SEQ ID NO: 67;
iii. a HCDR1 comprising an amino acid sequence as set forth in SEQ ID NO: 68, a HCDR2 comprising an amino acid sequence as set forth in SEQ ID NO: 69, and a HCDR3 comprising an amino acid sequence as set forth in SEQ ID NO: 70, a LCDR1 comprising an amino acid sequence as set forth in SEQ ID NO: 71, a LCDR2 comprising an amino acid sequence as set forth in SEQ ID NO: 72, and a LCDR3 comprising an amino acid sequence as set forth in SEQ ID NO: 73;
iv. a HCDR1 comprising an amino acid sequence as set forth in SEQ ID NO: 74, a HCDR2 comprising an amino acid sequence as set forth in SEQ ID NO: 75, and a HCDR3 comprising an amino acid sequence as set forth in SEQ ID NO: 76, a LCDR1 comprising an amino acid sequence as set forth in SEQ ID NO: 77, a LCDR2 comprising an amino acid sequence as set forth in SEQ ID NO: 78, and a LCDR3 comprising an amino acid sequence as set forth in SEQ ID NO: 79;
v. a HCDR1 comprising an amino acid sequence as set forth in SEQ ID NO: 188, a HCDR2 comprising an amino acid sequence as set forth in SEQ ID NO: 189, and a HCDR3 comprising an amino acid sequence as set forth in SEQ ID NO: 190, a LCDR1 comprising an amino acid sequence as set forth in SEQ ID NO: 191, a LCDR2 comprising an amino acid sequence as set forth in SEQ ID NO: 192, and a LCDR3 comprising an amino acid sequence as set forth in SEQ ID NO: 193;
vi. a HCDR1 comprising an amino acid sequence as set forth in SEQ ID NO: 251, a HCDR2 comprising an amino acid sequence as set forth in SEQ ID NO: 252, a HCDR3 comprising an amino acid sequence as set forth in SEQ ID NO: 253, a LCDR1 comprising an amino acid sequence as set forth in SEQ ID NO: 254, a LCDR2 comprising an amino acid sequence as set forth in SEQ ID NO: 255, and a LCDR3 comprising an amino acid sequence as set forth in SEQ ID NO: 256;
vii. a HCDR1 comprising an amino acid sequence as set forth in SEQ ID NO: 264, a HCDR2 comprising an amino acid sequence as set forth in SEQ ID NO: 265, a HCDR3 comprising an amino acid sequence as set forth in SEQ ID NO: 266, a LCDR1 comprising an amino acid sequence as set forth in SEQ ID NO: 267, a LCDR2 comprising an amino acid sequence as set forth in SEQ ID NO: 268, and a LCDR3 comprising an amino acid sequence as set forth in SEQ ID NO: 269;
viii. a HCDR1 comprising an amino acid sequence as set forth in SEQ ID NO: 276, a HCDR2 comprising an amino acid sequence as set forth in SEQ ID NO: 277, a HCDR3 comprising an amino acid sequence as set forth in SEQ ID NO: 278, a LCDR1 comprising an amino acid sequence as set forth in SEQ ID NO: 279, a LCDR2 comprising an amino acid sequence as set forth in SEQ ID NO: 280, and a LCDR3 comprising an amino acid sequence as set forth in SEQ ID NO: 281;
ix. a HCDR1 comprising an amino acid sequence as set forth in SEQ ID NO: 288, a HCDR2 comprising an amino acid sequence as set forth in SEQ ID NO: 289, a HCDR3 comprising an amino acid sequence as set forth in SEQ ID NO: 290, a LCDR1 comprising an amino acid sequence as set forth in SEQ ID NO: 291, a LCDR2 comprising an amino acid sequence as set forth in SEQ ID NO: 292, and a LCDR3 comprising an amino acid sequence as set forth in SEQ ID NO: 293;
x. a HCDR1 comprising an amino acid sequence as set forth in SEQ ID NO: 301, a HCDR2 comprising an amino acid sequence as set forth in SEQ ID NO: 302, a HCDR3 comprising an amino acid sequence as set forth in SEQ ID NO: 303, a LCDR1 comprising an amino acid sequence as set forth in SEQ ID NO: 304, a LCDR2 comprising an amino acid sequence as set forth in SEQ ID NO: 305, and a LCDR3 comprising an amino acid sequence as set forth in SEQ ID NO: 306; or
xi. a HCDR1 comprising an amino acid sequence as set forth in SEQ ID NO: 320, a HCDR2 comprising an amino acid sequence as set forth in SEQ ID NO: 321, a HCDR3 comprising an amino acid sequence as set forth in SEQ ID NO: 322, a LCDR1 comprising an amino acid sequence as set forth in SEQ ID NO: 323, a LCDR2 comprising an amino acid sequence as set forth in SEQ ID NO: 324, and a LCDR3 comprising an amino acid sequence as set forth in SEQ ID NO: 325.

45. The antibody or antigen-binding fragment thereof of any one of claims 36 to 44, wherein the second binding moiety comprises a VH region having an amino acid sequence as set forth in SEQ ID NOs: 1, 5, 9, 13, 14, 15, 16, 19, 147, 243, 257, 270, 282, 294, 313 or 326, or a homologous sequence thereof having at least 80% sequence identity to SEQ ID NOs: 1, 5, 9, 13, 14, 15, 16, 19, 147, 243, 257, 270, 282, 294, 313 or 326.

46. The antibody or antigen-binding fragment thereof of any one of claims 36 to 45, wherein the second binding moiety comprises a VL region having an amino acid sequence as set forth in SEQ ID NOs: 2, 6, 10, 17, 18, 20, 148, 247, 258, 271, 283, 295, 314 or 327, or a homologous sequence thereof having at least 80% sequence identity to SEQ ID NOs: 2, 6, 10, 17, 18, 20, 148, 247, 258, 271, 283, 295, 314 or 327.

47. The antibody or antigen-binding fragment thereof of any one of claims 36 to 46, wherein the second binding moiety comprises a VH/VL amino acid sequence pair selected from the group consisting of SEQ ID NOs: 1/2, 5/6, 9/10, 15/18, 13/18, 16/17, 14/18, 19/20, 147/148, 243/247, 257/258, 270/271, 282/283, 294/295, 313/314, and 326/327.

48. The antibody or antigen-binding fragment thereof of any one of claims 36 to 47, wherein the second binding moiety further comprises one or more amino acid residue substitutions or modifications yet retains specific binding affinity to the second target.

49. The antibody or antigen-binding fragment thereof of claim 48, wherein at least one of the substitutions or modifications is within one or more of the CDR sequences of the VH region or VL region of the second binding moiety.

50. The antibody or antigen-binding fragment thereof of claim 48, wherein at least one of the substitutions or modifications is within one or more of the non-CDR sequences of the VH region or VL region of the second binding moiety.

51. The antibody or antigen-binding fragment of claims 36 to 50, further comprising one or more non-natural amino acid (NNAA) substitution.

52. The antibody or antigen-binding fragment thereof of any one of the preceding claims, which is linked to one or more conjugate moieties.

53. The antibody or antigen-binding fragment thereof of claim 52, wherein the conjugate moiety comprises a clearance-modifying agent, a chemotherapeutic agent, a toxin, a radioactive isotope, a lanthanide, a detectable label, a DNA-alkylator, a topoisomerase inhibitor, a tubulin-binder, a purification moiety or other anticancer drugs.

54. The antibody or antigen-binding fragment thereof of claim 52 or 53, wherein the conjugate moiety is covalently attached either directly or via a linker.

55. The antibody or antigen-binding fragment thereof of claim 52 or 53, wherein the sequence of the second binding moiety is the same as the sequence of the third binding moiety.

56. A chimeric antigen receptor, comprising the antibody or antigen-binding fragment of any one of claims 1-55, a transmembrane region and an intracellular signal region.

57. The chimeric antigen receptor of claim 56, wherein the transmembrane region comprises a transmembrane region of CD3, CD4, CD8 or CD28.

58. The chimeric antigen receptor of claim 56, wherein the intracellular signal region is selected from the group consisting of: an intracellular signal region sequence of CD3, FcγRI, CD27, CD28, CD137, CD134, MyD88, CD40, CD278, TLRs, or a combination thereof.

59. The chimeric antigen receptor of any one of claims 56 to 58, wherein the chimeric antigen receptor is grafted onto an allogeneic cell, an autologous cell or a xenogeneic cell.

60. The chimeric antigen receptor of any one of claims 56 to 59, wherein the chimeric antigen receptor is grafted onto an immune effector cell.

61. The chimeric antigen receptor of any one of claims 56 to 60, wherein the chimeric antigen receptor is grafted onto a T cell, a natural killer cell, a macrophage cell, or a tumor-infiltrating lymphocyte.

62. A pharmaceutical composition comprising the antibody or an antigen-binding fragment thereof of any one of claims 1 to 55, or the chimeric antigen receptor of any one of claims 56 to 61, and one or more pharmaceutically acceptable carriers.

63. An isolated polynucleotide encoding the antibody or antigen-binding fragment thereof of any one of claims 1 to 55, and/or the chimeric antigen receptor of any one of claims 56 to 61.

64. A vector comprising the isolated polynucleotide of claim 63.

65. A host expression system comprising the vector of claim 64 or having the polynucleotide of claim 63 integrated into genome thereof.

66. The host expression system of claim 65, which is a microorganism, a yeast, or a mammalian cell, optionally, wherein the microorganism is selected from the group consisting of E. coli and B. subtilis, optionally wherein the yeast is Saccharomyces, and optionally wherein the mammalian cell is selected from the group consisting of COS, CHO-S, CHO-K1, HEK-293, and 3T3 cells.

67. A virus comprising the vector of claim 64.

68. A kit comprising the antibody or antigen-binding fragment thereof of any one of claims 1 to 55 and/or the chimeric antigen receptor of any one of claims 56 to 61 and/or the pharmaceutical composition of claim 62, and a second therapeutic agent.

69. A method of expressing the antibody or antigen-binding fragment thereof of any one of claims 1 to 55 or the chimeric antigen receptor of any one of claims 56 to 61, comprising culturing the host expression system of claim 62 under the condition at which the antibody or antigen-binding fragment of any one of claims 1 to 55 or the chimeric antigen receptor of any one of claims 56 to 61 is expressed.

70. A method of treating, preventing or alleviating a disease, disorder or condition in a subject, comprising administering to the subject a therapeutically effective amount of the antibody or antigen-binding fragment thereof of any one of claims 1 to 55 and/or the chimeric antigen receptor of any one of claims 56 to 61 and/or the pharmaceutical composition of claim 62.

71. The method of claim 70, wherein the disease, disorder or condition is an immune disease, an autoimmune disease (e.g., autoimmune encephalomyelitis, systemic lupus erythematosus, etc.), an inflammatory disease, a cancer or a neurological disease.

72. The method of claim 71, wherein the cancer is a solid tumor or hematologic tumor.

73. The method of any one of claims 70 to 72, wherein the disease, disorder or condition is selected from the group consisting of lung cancer (e.g., non-small-cell lung cancer (NSCLC), small cell lung cancer (SCLC), adenocarcinoma of the lung, or squamous cell carcinoma of the lung), peritoneal cancer, carcinoid cancer, bone cancer, pancreatic cancer, primitive neuroectodermal tumor, skin cancer, gallbladder cancer, cancer of the head or neck, squamous cell cancer, uterine cancer, ovarian cancer, rectal cancer, prostate cancer, bladder cancer (e.g., urothelial cancer), cancer of the anal region (e.g., anal squamous cell carcinoma), gastric or stomach cancer (e.g., gastrointestinal cancer), esophageal cancer, colon cancer, breast cancer, uterine cancer, liver cancer (e.g., hepatoblastoma, hepatocellular carcinoma/hepatoma, or hepatic carcinoma), cholangiocarcinoma, sarcoma, colorectal cancer, carcinoma of the fallopian tubes, salivary gland carcinoma, carcinoma of the cervix, endometrial or uterine carcinoma, osteosarcoma, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, cancer of the nasopharynx, sarcoma of soft tissue, polycythemia vera, cancer of the urethra, cancer of the penis, cancer of the kidney or ureter (e.g., rhabdoid tumor of the kidney), cutaneous T-cell lymphoma, medulloblastoma, nephroblastoma, myelodysplastic syndrome, chronic and non-chronic myeloproliferative disorder, choroid plexus papilloma, renal cell carcinoma, carcinoma of the renal pelvis, neoplasms of the central nervous system (CNS), soft tissue sarcoma (e.g., rhabdomyosarcoma, fibrosarcoma, Kaposi's sarcoma), spinal axis tumors, glioma (e.g., ependymoma, astrocytoma, anaplastic astrocytoma, oligodendroglioma, eye cancer (e.g., retinoblastoma), brain stem glioma, or mixed glioma such as oligoastrocytoma), brain tumor (e.g., glioblastoma/glioblastoma multiforme (GBM), non-glioblastoma brain tumor, or meningioma), melanoma (e.g., cutaneous or intraocular melanoma), thrombocythemia, mesothelioma, mycosis fungoides, Sezary syndrome, idiopathic myelofibrosis, solitary plasmacytoma, vestibular schwannoma, Ewing's sarcoma, chondrosarcoma, MYH associated polyposis, pituitary adenoma, pediatric cancers such as pediatric sarcomas (e.g., neuroblastoma, rhabdomyosarcoma, and osteosarcoma), hematological cancer, Hodgkin's lymphoma, non-Hodgkin's lymphoma, leukemia (e.g., lymphocytic/lymphoblastic leukemia), chronic or acute leukemia, mast cell leukemia, lymphocytic lymphomas, primary CNS lymphoma, chronic lymphocytic leukemia (CLL), acute lymphocytic leukemia (ALL), chronic myeloid leukemia (CML), acute myeloid leukemia (AML), chronic myelomonocytic leukemia (CMML), chronic lymphoblastic leukemia, acute lymphoblastic leukemia, hairy cell leukemia (HCL), Burkitt's lymphoma (BL), multiple myeloma (e.g., relapsed or refractory multiple myeloma), T or B cell lymphoma, mantle cell lymphoma (MCL) (e.g., relapsed or refractory mantle cell lymphoma), malignant melanoma, diffuse large B cell lymphoma (DLBCL), DLBCL that results from follicular lymphoma, high-grade B-cell lymphoma, primary mediastinal large B-cell lymphoma, follicular lymphoma (FL), and primary mediastinal B-cell lymphoma.

74. The method of any one of claims 70 to 72, wherein the subject is human.

75. The method of any one of claims 70 to 74, wherein the administration is through a parenteral route comprising subcutaneous, intraperitoneal, intravenous, intramuscular, or intradermal injection; or a non-parenteral route comprising transdermal, oral, intranasal, intraocular, sublingual, rectal, or topical.

76. The method of any one of claims 70 to 75, wherein the method further includes administering to the subject in need thereof an additional therapeutic agent.

77. The method of claim 76, wherein the additional therapeutic agent is selected from the group consisting of: an active agent, an imaging agent, a cytotoxic agent, an angiogenesis inhibitor, a kinase inhibitor, a co-stimulation molecule agonist, a co-inhibition molecule blocker, an adhesion molecule blocker, an anti-cytokine antibody or functional fragment thereof, a detectable label or reporter, an antimicrobial, a gene editing agent, a beta agonist, an viral RNA inhibitor, a polymerase inhibitor, an interferon, and a microRNA.

78. The method of claim 76, wherein the additional therapeutic agent is administered to the subject in need before, after or simultaneously with the antibody or antigen-binding fragment thereof of any one of claims 1 to 55 and/or the chimeric antigen receptor of any one of claims 56 to 61 and/or the pharmaceutical composition of claim 62.

79. A method of activating a T cell expressing CD3 or another target other than CD3 in vivo or in vitro, comprising contacting the T cell with the antibody or antigen-binding fragment thereof of any one of claims 1 to 55 and/or the chimeric antigen receptor of any one of claims 56 to 61 and/or the pharmaceutical composition of claim 59.

80. A method of modulating activity of CD3 or another target other than CD3 in a cell expressing CD3 or another target other than CD3, comprising exposing the cell to the antibody or antigen-binding fragment thereof of any one of claims 1 to 55 and/or the chimeric antigen receptor of any one of claims 56 to 61 and/or the pharmaceutical composition of claim 59.

81. A method of promoting in vivo or in vitro processing of a second antigen by a CD3-expressing T cell, comprising contacting the CD3-expressing T cell with the antibody or antigen-binding fragment thereof of any of claims 1 to 55, wherein the antibody or antigen-binding fragment thereof is capable of binding to both the CD3-expressing T cell and the second antigen thereby bringing both in close proximity.

82. A method of detecting presence or amount of CD3 or another target other than CD3 in a sample, comprising contacting the sample with the antibody or antigen-binding fragment thereof of any one of claims 1 to 55 and/or the chimeric antigen receptor of any one of claims 56 to 61 and/or the pharmaceutical composition of claim 62, and determining the presence or the amount of CD3 or another target other than CD3 in the sample.

83. A method of diagnosing a disease, disorder or condition related to CD3 or another target other than CD3 in a subject, comprising: a) contacting a sample obtained from the subject with the antibody or antigen-binding fragment thereof of any one of claims 1 to 55 and/or the chimeric antigen receptor of any one of claims 56 to 61 and/or the pharmaceutical composition of claim 62; b) determining presence or amount of CD3 or another target other than CD3 in the sample; and c) correlating the presence or the amount of CD3 or another target other than CD3 to existence or status of the disease, disorder or condition in the subject.

84. Use of the antibody or antigen-binding fragment thereof of any of any one of claims 1 to 55 and/or the chimeric antigen receptor of any one of claims 56 to 61 and/or the pharmaceutical composition of claim 62 in the manufacture of a medicament for treating a disease, disorder or condition related to CD3 or another target other than CD3 in a subject.

85. Use of the antibody or antigen-binding fragment thereof of any one of claims 1 to 55 and/or the chimeric antigen receptor of any one of claims 56 to 61 and/or the pharmaceutical composition of claim 62 in the manufacture of a diagnostic reagent for diagnosing a disease, disorder or condition related to CD3 or another target other than CD3.

86. A kit comprising the antibody or antigen-binding fragment thereof of any one of claims 1 to 54 and/or the chimeric antigen receptor of any one of claims 56 to 61 and/or the pharmaceutical composition of claim 62, useful in detecting CD3 or another target other than CD3, optionally recombinant CD3, CD3 expressed on cell surface, or CD3-expresing cells.


SEQUENCE LISTING (333 sequences)

SEQ ID NO: 1 - protein, 118 aa, synthetic construct
EVQLQQSGPELVKPGASVKMSCKASGYTFTSYVIHWVKQKTGQGLEWIGYFNPYNDGTKYNEEFKGKATLTSDKSSNTAYMELNSLTSDDSAVYFCARGGVRRYFDVWGAGTTVTVSS

SEQ ID NO: 2 - protein, 107 aa, synthetic construct
DIQMTQSPSSLSASLGERVSLTCRASQDIGSNLNWLQQEPDGTIKRLIYATFGLDSGVPKRFSGSRSGSDYSLTISSLESEDFVDYYCQQYASFPYTFGGGTKLEIK

SEQ ID NO: 3 - protein, 120 aa, synthetic construct
EVQLQLSGPELVKPGASVKMSCKASGYTFSSYVIHWVKQKPGQGLEWIGYINPYNDVIKYNEKFKGKATLTSDKSSSTAYMELSSLTSEDSAVYYCARDSYYSYDGFAYWGQGTLVTVSA

SEQ ID NO: 4 - protein, 106 aa, synthetic construct
QIVLTQSPAIMSASPGEKVTMTCSASSSVSYMHWYQQKSGTSPKRWIYDTSKLASGVPARFSGSGSGTSYSLTISSMEAEDAATYYCQQWSSNPPTFGGGTKVEIK

SEQ ID NO: 5 - protein, 118 aa, synthetic construct
QVQLVQSGAEVKKPGASVKVSCKASGYSFTGYTMNWVRQAPGQGLEWMGLINPYNSDTNYAQKLQGRVTMTTDTSTSTAYMELRSLRSDDTAVYYCARVALRVALDYWGQGTLVTVSS

SEQ ID NO: 6 - protein, 107 aa, synthetic construct
DIQMTQSPSSLSASVGDRVTITCKASQNVATHVGWYQQKPGKAPKRLIYSASYRYSGVPSRFSGSGSGTEFTLTISNLQPEDFATYYCQQYNRYPYTFGQGTKLEIK

SEQ ID NO: 7 - protein, 125 aa, synthetic construct
EVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCARHGNFGNSYVSWFAYWGQGTLVTVSS

SEQ ID NO: 8 - protein, 109 aa, synthetic construct
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVL

SEQ ID NO: 9 - protein, 116 aa, synthetic construct
EVQLVESGGGLVQPGRSLKLSCAASGFSFSNYGMAWVRQAATKGLEWVASISTGGGNTYYRDSVKGRFIVSRDNAKNTQYLQMDSLRSEDTATYYCTRHDRGGLYWGQGVMVTVSS

SEQ ID NO: 10 - protein, 112 aa, synthetic construct
DIVMTQAPLSVSVTPGESASISCRSSKSLLHSNGITYVYWYFQKPGKSPQVLIYRMSNLASGVPDRFSGSGSETDFTLKISRVEAEDVGIYHCGQLLENPYTFGAGTELELK

SEQ ID NO: 11 - protein, 125 aa, synthetic construct
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSS

SEQ ID NO: 12 - protein, 109 aa, synthetic construct
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 13 - protein, 118 aa, synthetic construct
EVQLVQSGAEVKKPGASVKVSCKASGYTFTSYVIHWVRQAPGQGLEWMGYFNPYNDGTKYNEEFKGRVTMTTDTSTSTAYMELRSLRSDDTAVYYCARGGVRRYFDVWGQGTTVTVSS

SEQ ID NO: 14 - protein, 118 aa, synthetic construct
EVQLVQSGAEVKKPGASVKVSCKASGYTFTSYVIHWVRQAPGQGLEWIGYFNPYNDGTKYNEEFKGRATMTTDTSTSTAYMELRSLRSDDTAVYYCARGGVRRYFDVWGQGTTVTVSS

SEQ ID NO: 15 - protein, 118 aa, synthetic construct
EVQLVQSGAEVKKPGASVKVSCKASGYTFTSYVIHWVRQAPGQGLEWMGYFNPYNDGTKYNEEFKGRVTLTSDKSTSTAYMELRSLRSDDTAVYYCARGGVRRYFDVWGQGTTVTVSS

SEQ ID NO: 16 - protein, 118 aa, synthetic construct
EVQLVQSGAEVKKPGASVKVSCKASGYTFTSYVIHWVKQAPGQGLEWIGYFNPYNDGTKYNEEFKGKATLTTDKSTSTAYMELRSLRSDDTAVYYCARGGVRRYFDVWGQGTTVTVSS

SEQ ID NO: 17 - protein, 107 aa, synthetic construct
DIQMTQSPSSLSASVGDRVTITCRASQDIGSNLNWLQQKPGKAIKRLIYATFGLDSGVPSRFSGSGSGTDYTLTISSLQPEDFATYYCQQYASFPYTFGGGTKVEIK

SEQ ID NO: 18 - protein, 107 aa, synthetic construct
DIQMTQSPSSLSASVGDRVTITCRASQDIGSNLNWLQQKPGKAIKRLIYATFGLDSGVPSRFSGSRSGTDFTLTISSLQPEDFATYYCQQYASFPYTFGGGTKVEIK

SEQ ID NO: 19 - protein, 119 aa, synthetic construct
QVQLVESGPGVVQPGRSLRISCAVSGFSVTNYGVHWVRQPPGKGLEWLGVIWAGGITNYNSAFMSRLTISKDNSKNTVYLQMNSLRAEDTAMYYCASRGGHYGYALDYWGQGTLVTVSS

SEQ ID NO: 20 - protein, 104 aa, synthetic construct
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGQGTKLEIK

SEQ ID NO: 21 - protein, 120 aa, synthetic construct
EVQLVQSGAEVKKPGSSVKVSCKASGGTFSSYVIHWVRQAPGQGLEWMGYINPYNDVIKYNEKFKGRVTITADKSTSTAYMELSSLRSEDTAVYYCARDSYYSYDGFAYWGQGTLVTVSS

SEQ ID NO: 22 - protein, 120 aa, synthetic construct
EVQLVQSGAEVKKPGSSVKVSCKASGYTFSSYVIHWVRQAPGQGLEWMGYINPYNDVIKYNEKFKGRVTLTSDKSTSTAYMELSSLRSEDTAVYYCARDSYYSYDGFAYWGQGTLVTVSS

SEQ ID NO: 23 - protein, 120 aa, synthetic construct
EVQLVQSGAEVKKPGSSVKVSCKASGYTFSSYVIHWVRQAPGQGLEWIGYINPYNDVIKYNEKFKGRATLTSDKSTSTAYMELSSLRSEDTAVYYCARDSYYSYDGFAYWGQGTLVTVSS

SEQ ID NO: 24 - protein, 106 aa, synthetic construct
EIVLTQSPDFQSVTPKEKVTITCSASSSVSYMHWYQQKPDQSPKRLIYDTSKLASGVPSRFSGSGSGTDFTLTINSLEAEDAATYYCQQWSSNPPTFGGGTKVEIK

SEQ ID NO: 25 - protein, 106 aa, synthetic construct
EIVLTQSPDFQSVTPKEKVTITCSASSSVSYMHWYQQKPDQSPKRWIYDTSKLASGVPSRFSGSGSGTDYTLTINSLEAEDAATYYCQQWSSNPPTFGGGTKVEIK

SEQ ID NO: 26 - protein, 120 aa, synthetic construct
EVQLVQSGAEVKKPGSSVKVSCKASGYTFSSYVIHWVRQAPGQGLEWMGYINPYNDVIKYNEKFKGRVTLTSDKSTSTAYMELSSLRSEDTAVYYCARESYYSYDGFAYWGQGTLVTVSS

SEQ ID NO: 27 - protein, 120 aa, synthetic construct
EVQLVQSGAEVKKPGSSVKVSCKASGYTFSSYVIHWVRQAPGQGLEWMGYINPYNDVIKYNEKFKGRVTLTSDKSTSTAYMELSSLRSEDTAVYYCARDSFYSYDGFAYWGQGTLVTVSS

SEQ ID NO: 28 - protein, 120 aa, synthetic construct
EVQLVQSGAEVKKPGSSVKVSCKASGYTFSSYVIHWVRQAPGQGLEWMGYINPYNDVIKYNEKFKGRVTLTSDKSTSTAYMELSSLRSEDTAVYYCARDSYYSYDAFAYWGQGTLVTVSS

SEQ ID NO: 29 - protein, 120 aa, synthetic construct
EVQLVQSGAEVKKPGSSVKVSCKASGYTFSSYVIHWVRQAPGQGLEWMGYINPGNDVIKYNEKFKGRVTLTSDKSTSTAYMELSSLRSEDTAVYYCARDSYYSYDGFAYWGQGTLVTVSS

SEQ ID NO: 30 - protein, 120 aa, synthetic construct
EVQLVQSGAEVKKPGSSVKVSCKASGYTFSSYVIHWVRQAPGQGLEWMGYINPYNDVIKYNEKFKGRVTLTSDKSTSTAYMELSSLRSEDTAVYYCARDSYYSYRGFAYWGQGTLVTVSS

SEQ ID NO: 31 - protein, 5 aa, synthetic construct
SYVIH

SEQ ID NO: 32 - protein, 17 aa, synthetic construct
YINPYNDVIKYNEKFKG

SEQ ID NO: 33 - protein, 17 aa, synthetic construct
YINPGNDVIKYNEKFKG

SEQ ID NO: 34 - protein, 17 aa, synthetic construct
  VARIANT 5: note = X=Y or G
YINPXNDVIKYNEKFKG

SEQ ID NO: 35 - protein, 11 aa, synthetic construct
DSYYSYDGFAY

SEQ ID NO: 36 - protein, 11 aa, synthetic construct
ESYYSYDGFAY

SEQ ID NO: 37 - protein, 11 aa, synthetic construct
DSFYSYDGFAY

SEQ ID NO: 38 - protein, 11 aa, synthetic construct
DSYYSYDAFAY

SEQ ID NO: 39 - protein, 11 aa, synthetic construct
DSYYSYRGFAY

SEQ ID NO: 40 - protein, 11 aa, synthetic construct
  VARIANT 1: note = X= D or E
  VARIANT 3: note = X=Y or F
  VARIANT 7: note = X=D or R
  VARIANT 8: note = X=G or A
XSXYSYXXFAY

SEQ ID NO: 41 - protein, 10 aa, synthetic construct
SASSSVSYMH

SEQ ID NO: 42 - protein, 7 aa, synthetic construct
DTSKLAS

SEQ ID NO: 43 - protein, 9 aa, synthetic construct
QQWSSNPPT

SEQ ID NO: 44 - protein, 5 aa, synthetic construct
TYAMN

SEQ ID NO: 45 - protein, 19 aa, synthetic construct
RIRSKYNNYATYYAASVKG

SEQ ID NO: 46 - protein, 14 aa, synthetic construct
HGNFGNSYVSWFAY

SEQ ID NO: 47 - protein, 14 aa, synthetic construct
RSSTGAVTTSNYAN

SEQ ID NO: 48 - protein, 7 aa, synthetic construct
GTNKRAP

SEQ ID NO: 49 - protein, 9 aa, synthetic construct
ALWYSNLWV

SEQ ID NO: 50 - protein, 5 aa, synthetic construct
TYAMN

SEQ ID NO: 51 - protein, 19 aa, synthetic construct
RIRSKYNNYATYYADSVKG

SEQ ID NO: 52 - protein, 14 aa, synthetic construct
HGNFGNSYVSWFAY

SEQ ID NO: 53 - protein, 14 aa, synthetic construct
GSSTGAVTTSNYAN

SEQ ID NO: 54 - protein, 7 aa, synthetic construct
GTNKRAP

SEQ ID NO: 55 - protein, 9 aa, synthetic construct
ALWYSNLWV

SEQ ID NO: 56 - protein, 5 aa, synthetic construct
SYVIH

SEQ ID NO: 57 - protein, 17 aa, synthetic construct
YFNPYNDGTKYNEEFKG

SEQ ID NO: 58 - protein, 9 aa, synthetic construct
GGVRRYFDV

SEQ ID NO: 59 - protein, 11 aa, synthetic construct
RASQDIGSNLN

SEQ ID NO: 60 - protein, 7 aa, synthetic construct
ATFGLDS

SEQ ID NO: 61 - protein, 9 aa, synthetic construct
QQYASFPYT

SEQ ID NO: 62 - protein, 5 aa, synthetic construct
GYTMN

SEQ ID NO: 63 - protein, 17 aa, synthetic construct
LINPYNSDTNYAQKLQG

SEQ ID NO: 64 - protein, 9 aa, synthetic construct
VALRVALDY

SEQ ID NO: 65 - protein, 11 aa, synthetic construct
KASQNVATHVG

SEQ ID NO: 66 - protein, 7 aa, synthetic construct
SASYRYS

SEQ ID NO: 67 - protein, 9 aa, synthetic construct
QQYNRYPYT

SEQ ID NO: 68 - protein, 5 aa, synthetic construct
NYGMA

SEQ ID NO: 69 - protein, 17 aa, synthetic construct
SISTGGGNTYYRDSVKG

SEQ ID NO: 70 - protein, 7 aa, synthetic construct
HDRGGLY

SEQ ID NO: 71 - protein, 16 aa, synthetic construct
RSSKSLLHSNGITYVY

SEQ ID NO: 72 - protein, 7 aa, synthetic construct
RMSNLAS

SEQ ID NO: 73 - protein, 9 aa, synthetic construct
GQLLENPYT

SEQ ID NO: 74 - protein, 5 aa, synthetic construct
NYGVH

SEQ ID NO: 75 - protein, 16 aa, synthetic construct
VIWAGGITNYNSAFMS

SEQ ID NO: 76 - protein, 11 aa, synthetic construct
RGGHYGYALDY

SEQ ID NO: 77 - protein, 11 aa, synthetic construct
KASQSVSNDVT

SEQ ID NO: 78 - protein, 7 aa, synthetic construct
SASNRYS

SEQ ID NO: 79 - protein, 6 aa, synthetic construct
QQDYSS

SEQ ID NO: 80 - protein, 119 aa, synthetic construct
QVQLQQPGTELVKPGTSVKLSCKASGYTFTSYWMHWVKQRPGQGLEWIGNINPSNGATNYNEKFKSKATLTVDKSSSTAYMQLSSLTSEDSAVYYCARDRSSQYYFDYWGQGTTLTVSS

SEQ ID NO: 81 - protein, 112 aa, synthetic construct
DIVMSQSPSSLAVSAGEKVTMSCKSSQSLLNSRSRKNYLAWYQQKPGQSPKLLIYWASTRESGVPDRFTGSGSGTDFTLTISSVQAEDLAVYYCTQSYTLRTFGGGTKLEIK

SEQ ID NO: 82 - protein, 119 aa, synthetic construct
EVQLVQSGAEVKKPGSSVKVSCKASGYTFTSYWMHWVRQAPGQGLEWMGNINPSSGATNYNEKFKSRVTLTVDKSTSTAYMELSSLRSEDTAVYYCARDRSSQYYFDYWGQGTTVTVSS

SEQ ID NO: 83 - protein, 119 aa, synthetic construct
EVQLVQSGAEVKKPGSSVKVSCKASGYTFTRYWMHWVRQAPGQGLEWMGNINPSSGATNYNEKFKSRVTLTVDKSTSTAYMELSSLRSEDTAVYYCARDRSSQYYFDYWGQGTTVTVSS

SEQ ID NO: 84 - protein, 119 aa, synthetic construct
EVQLVQSGAEVKKPGSSVKVSCKASGYTFTSYWMHWVRQAPGQGLEWMGNINPRSGATNYNEKFKSRVTLTVDKSTSTAYMELSSLRSEDTAVYYCARDRSSQYYFDYWGQGTTVTVSS

SEQ ID NO: 85 - protein, 119 aa, synthetic construct
EVQLVQSGAEVKKPGSSVKVSCKASGYTFTSYWMHWVRQAPGQGLEWMGNINPSSGATNYNEKFKSRVTLTVDKSTSTAYMELSSLRSEDTAVYYCARDRSQQYYFDYWGQGTTVTVSS

SEQ ID NO: 86 - protein, 119 aa, synthetic construct
EVQLVQSGAEVKKPGSSVKVSCKASGYTFTSYWMHWVRQAPGQGLEWMGNINPSSGATNYNEKFKSRVTLTVDKSTSTAYMELSSLRSEDTAVYYCARDQSSQYYFDYWGQGTTVTVSS

SEQ ID NO: 87 - protein, 112 aa, synthetic construct
DIVMTQSPDSLAVSLGERATINCKSSQSLLNSRSRKNYLAWYQQKPGQPPKLLIYWASTRESGVPDRFSGSGSGTDFTLTISSLQAEDVAVYYCTQSYTLRTFGGGTKVEIK

SEQ ID NO: 88 - protein, 112 aa, synthetic construct
DIVMTQSPDSLAVSLGERATINCKSSQSLLNTRSRKNYLAWYQQKPGQPPKLLIYWASTRESGVPDRFSGSGSGTDFTLTISSLQAEDVAVYYCTQSYTLRTFGGGTKVEIK

SEQ ID NO: 89 - protein, 112 aa, synthetic construct
DIVMTQSPDSLAVSLGERATINCKSSQSLLNRRSRKNYLAWYQQKPGQPPKLLIYWASTRESGVPDRFSGSGSGTDFTLTISSLQAEDVAVYYCTQSYTLRTFGGGTKVEIK

SEQ ID NO: 90 - protein, 112 aa, synthetic construct
DIVMTQSPDSLAVSLGERATINCKSSQSLLNSRSRRNYLAWYQQKPGQPPKLLIYWASTRESGVPDRFSGSGSGTDFTLTISSLQAEDVAVYYCTQSYTLRTFGGGTKVEIK

SEQ ID NO: 91 - protein, 112 aa, synthetic construct
DIVMTQSPDSLAVSLGERATINCKSSQSLLYSRSRKNYLAWYQQKPGQPPKLLIYWASTRESGVPDRFSGSGSGTDFTLTISSLQAEDVAVYYCTQSYTLRTFGGGTKVEIK

SEQ ID NO: 92 - protein, 112 aa, synthetic construct
DIVMTQSPDSLAVSLGERATINCKSSQSLLNSRSKKNYLAWYQQKPGQPPKLLIYWASTRESGVPDRFSGSGSGTDFTLTISSLQAEDVAVYYCTQSYTLRTFGGGTKVEIK

SEQ ID NO: 93 - protein, 112 aa, synthetic construct
DIVMTQSPDSLAVSLGERATINCKSSQSLLNSASRKNYLAWYQQKPGQPPKLLIYWASTRESGVPDRFSGSGSGTDFTLTISSLQAEDVAVYYCTQSYTLRTFGGGTKVEIK

SEQ ID NO: 94 - protein, 112 aa, synthetic construct
DIVMTQSPDSLAVSLGERATINCKSSQSLLNSRSSKNYLAWYQQKPGQPPKLLIYWASTRESGVPDRFSGSGSGTDFTLTISSLQAEDVAVYYCTQSYTLRTFGGGTKVEIK

SEQ ID NO: 95 - protein, 112 aa, synthetic construct
DIVMTQSPDSLAVSLGERATINCKSSQSLLNSRSRKKYLAWYQQKPGQPPKLLIYWASTRESGVPDRFSGSGSGTDFTLTISSLQAEDVAVYYCTQSYTLRTFGGGTKVEIK

SEQ ID NO: 96 - protein, 5 aa, synthetic construct
SYWMH

SEQ ID NO: 97 - protein, 5 aa, synthetic construct
RYWMH

SEQ ID NO: 98 - protein, 5 aa, synthetic construct
  VARIANT 1: note = X=S or R
XYWMH

SEQ ID NO: 99 - protein, 17 aa, synthetic construct
NINPSNGATNYNEKFKS

SEQ ID NO: 100 - protein, 17 aa, synthetic construct
NINPSSGATNYNEKFKS

SEQ ID NO: 101 - protein, 17 aa, synthetic construct
NINPRSGATNYNEKFKS

SEQ ID NO: 102 - protein, 17 aa, synthetic construct
  VARIANT 5: note = X=S or R
  VARIANT 6: note = X=N or S
NINPXXGATNYNEKFKS

SEQ ID NO: 103 - protein, 10 aa, synthetic construct
DRSSQYYFDY

SEQ ID NO: 104 - protein, 10 aa, synthetic construct
DRSQQYYFDY

SEQ ID NO: 105 - protein, 10 aa, synthetic construct
DQSSQYYFDY

SEQ ID NO: 106 - protein, 10 aa, synthetic construct
  VARIANT 2: note = X=R or Q
  VARIANT 4: note = X=S or Q
DXSXQYYFDY

SEQ ID NO: 107 - protein, 17 aa, synthetic construct
KSSQSLLNSRSRKNYLA

SEQ ID NO: 108 - protein, 17 aa, synthetic construct
KSSQSLLNTRSRKNYLA

SEQ ID NO: 109 - protein, 17 aa, synthetic construct
KSSQSLLNRRSRKNYLA

SEQ ID NO: 110 - protein, 17 aa, synthetic construct
KSSQSLLNSRSRRNYLA

SEQ ID NO: 111 - protein, 17 aa, synthetic construct
KSSQSLLYSRSRKNYLA

SEQ ID NO: 112 - protein, 17 aa, synthetic construct
KSSQSLLNSRSKKNYLA

SEQ ID NO: 113 - protein, 17 aa, synthetic construct
KSSQSLLNSASRKNYLA

SEQ ID NO: 114 - protein, 17 aa, synthetic construct
KSSQSLLNSRSSKNYLA

SEQ ID NO: 115 - protein, 17 aa, synthetic construct
KSSQSLLNSRSRKKYLA

SEQ ID NO: 116 - protein, 17 aa, synthetic construct
  VARIANT 8: note = X=N or Y
  VARIANT 9: note = X=S, T or R
  VARIANT 10: note = X=R or A
  VARIANT 12: note = X=R, K or S
  VARIANT 13: note = X=K or R
  VARIANT 14: note = X=N or K
KSSQSLLXXXSXXXYLA

SEQ ID NO: 117 - protein, 7 aa, synthetic construct
WASTRES

SEQ ID NO: 118 - protein, 8 aa, synthetic construct
TQSYTLRT

SEQ ID NO: 119 - protein, 120 aa, synthetic construct
EVQLVQSGAEVKKPGSSVKVSCKASGYTFSSYVIHWVRQAPGQCLEWMGYINPYNDVIKYNEKFKGRVTLTSDKSTSTAYMELSSLRSEDTAVYYCARDSYYSYDGFAYWGQGTLVTVSS

SEQ ID NO: 120 - protein, 120 aa, synthetic construct
EVQLVQSGAEVKKPGSSVKVSCKASGYTFSSYVIHWVRQAPGQCLEWMGYINPYSDVIKYNEKFKGRVTLTSDKSTSTAYMELSSLRSEDTAVYYCARDSYYSYDGFAYWGQGTLVTVSS

SEQ ID NO: 121 - protein, 17 aa, synthetic construct
YINPYSDVIKYNEKFKG

SEQ ID NO: 122 - protein, 120 aa, synthetic construct
EVQLVQSGAEVKKPGSSVKVSCKASGYTFSSYVIHWVRQAPGQCLEWMGYINPYNGVIKYNEKFKGRVTLTSDKSTSTAYMELSSLRSEDTAVYYCARDSYYSYDGFAYWGQGTLVTVSS

SEQ ID NO: 123 - protein, 17 aa, synthetic construct
YINPYNGVIKYNEKFKG

SEQ ID NO: 124 - protein, 120 aa, synthetic construct
EVQLVQSGAEVKKPGSSVKVSCKASGYTFSSYVIHWVRQAPGQCLEWMGYINPYNDVIKYNEKFKGRVTLTSDKSTSTAYMELSSLRSEDTAVYYCARDSFYSYDGFAYWGQGTLVTVSS

SEQ ID NO: 125 - protein, 120 aa, synthetic construct
EVQLVQSGAEVKKPGSSVKVSCKASGYTFSSYVIHWVRQAPGQCLEWMGYINPYNDVIKYNEKFKGRVTLTSDKSTSTAYMELSSLRSEDTAVYYCARDSYYSYRGFAYWGQGTLVTVSS

SEQ ID NO: 126 - protein, 120 aa, synthetic construct
EVQLVQSGAEVKKPGSSVKVSCKASGYTFSSYVIHWVRQAPGQCLEWMGYINPYNDVIKYNEKFKGRVTLTSDKSTSTAYMELSSLRSEDTAVYYCARESFSSYDGFAYWGQGTLVTVSS

SEQ ID NO: 127 - protein, 11 aa, synthetic construct
ESFSSYDGFAY

SEQ ID NO: 128 - protein, 120 aa, synthetic construct
EVQLVQSGAEVKKPGSSVKVSCKASGYTFSSYVIHWVRQAPGQCLEWMGYINPGNGVIKYNEKFKGRVTLTSDKSTSTAYMELSSLRSEDTAVYYCARDSYSSYDGFAYWGQGTLVTVSS

SEQ ID NO: 129 - protein, 17 aa, synthetic construct
YINPGNGVIKYNEKFKG

SEQ ID NO: 130 - protein, 11 aa, synthetic construct
DSYSSYDGFAY

SEQ ID NO: 131 - protein, 120 aa, synthetic construct
EVQLVQSGAEVKKPGSSVKVSCKASGYTFSSYVIHWVRQAPGQCLEWMGYINPYSDVIKYNEKFKGRVTLTSDKSTSTAYMELSSLRSEDTAVYYCARESFYSYDGFAYWGQGTLVTVSS

SEQ ID NO: 132 - protein, 5 aa, synthetic construct
  VARIANT 1: note = X=S or D
XYVIH

SEQ ID NO: 133 - protein, 11 aa, synthetic construct
ESFYSYDGFAY

SEQ ID NO: 134 - protein, 120 aa, synthetic construct
EVQLVQSGAEVKKPGSSVKVSCKASGYTFSSYVIHWVRQAPGQCLEWMGYINPYNDVIKYNEKFKGRVTLTSDKSTSTAYMELSSLRSEDTAVYYCARDSYYSYEAFAYWGQGTLVTVSS

SEQ ID NO: 135 - protein, 11 aa, synthetic construct
DSYYSYEAFAY

SEQ ID NO: 136 - protein, 120 aa, synthetic construct
EVQLVQSGAEVKKPGSSVKVSCKASGYTFSSYVIHWVRQAPGQCLEWMGYINPYNDVIKYNEKFKGRVTLTSDKSTSTAYMELSSLRSEDTAVYYCARDSYYSYRAFAYWGQGTLVTVSS

SEQ ID NO: 137 - protein, 11 aa, synthetic construct
DSYYSYRAFAY

SEQ ID NO: 138 - protein, 120 aa, synthetic construct
EVQLVQSGAEVKKPGSSVKVSCKASGYTFSSYVIHWVRQAPGQCLEWMGYINPYNDVIKYNEKFKGRVTLTSDKSTSTAYMELSSLRSEDTAVYYCARESYYSYDGFAYWGQGTLVTVSS

SEQ ID NO: 139 - protein, 120 aa, synthetic construct
EVQLVQSGAEVKKPGSSVKVSCKASGYTFSDYVIHWVRQAPGQCLEWMGYINPYNDVIKYNEKFKGRVTLTSDKSTSTAYMELSSLRSEDTAVYYCARDSYYSYDGFAYWGQGTLVTVSS

SEQ ID NO: 140 - protein, 5 aa, synthetic construct
DYVIH

SEQ ID NO: 141 - protein, 106 aa, synthetic construct
EIVLTQSPDFQSVTPKEKVTITCSASSSVSYMHWYQQKPDQSPKRLIYDTSKLASGVPSRFSGSGSGTDFTLTINSLEAEDAATYYCQQWRSSPPTFGCGTKVEIK

SEQ ID NO: 142 - protein, 9 aa, synthetic construct
QQWRSSPPT

SEQ ID NO: 143 - protein, 17 aa, synthetic construct
  VARIANT 5: note = X=Y or G
  VARIANT 6: note = X=N or S
  VARIANT 7: note = X=D or G
YINPXXXVIKYNEKFKG

SEQ ID NO: 144 - protein, 11 aa, synthetic construct
  VARIANT 1: note = X=D or E
  VARIANT 3: note = X=Y or F
  VARIANT 4: note = X=Y or S
  VARIANT 7: note = X=D, R or E
  VARIANT 8: note = X=G or A
XSXXSYXXFAY

SEQ ID NO: 145 - protein, 119 aa, synthetic construct
QVQLVQSGAEVKKPGSSVKVSCKASGFAFTDYYIHWVRQAPGQGLEWMGWISPGNVNTKYNENFKGRVTITADKSTSTAYMELSSLRSEDTAVYYCARDGYSLYYFDYWGQGTLVTVSS

SEQ ID NO: 146 - protein, 112 aa, synthetic construct
DIVMTQSPDSLAVSLGERATINCKSSQSLLNSRTRKNYLAWYQQKPGQPPKLLIYWASTRQSGVPDRFSGSGSGTDFTLTISSLQAEDVAVYYCTQSHTLRTFGGGTKVEIK

SEQ ID NO: 147 - protein, 120 aa, synthetic construct
EVQLVESGGGLVQPGGSLRLSCAASGFSLSSSYWISWVRQAPGKGLEWIGSIDSGSVGITYYATWVKGRFTISRDNSKNTLYLQMNSLRAEDTAVYYCARHGDNWALDLWGQGTLVTVLE

SEQ ID NO: 148 - protein, 110 aa, synthetic construct
DIQMTQSPSTLSASVGDRVTITCRASQSISSWLAWYQQKPGKAPKLLIYKASTLASGVPSRFSGSGSGTEFTLTISSLQPDDFATYYCQHGYIRGDLDNVFGGGTKVEIK

SEQ ID NO: 149 - protein, 9 aa, synthetic construct
  VARIANT 4: note = X=S or R
  VARIANT 6: note = X=N or S
QQWXSXPPT

SEQ ID NO: 150 - protein, 107 aa, synthetic construct [sequence omitted]

SEQ ID NO: 151 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 152 - protein, 448 aa, synthetic construct
EVQLVQSGAEVKKPGASVKVSCKASGYTFTSYVIHWVRQAPGQGLEWMGYFNPYNDGTKYNEEFKGRVTLTSDKSTSTAYMELRSLRSDDTAVYYCARGGVRRYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 153 - protein, 714 aa, synthetic construct
EVQLVQSGAEVKKPGASVKVSCKASGYTFTSYVIHWVRQAPGQGLEWMGYFNPYNDGTKYNEEFKGRVTLTSDKSTSTAYMELRSLRSDDTAVYYCARGGVRRYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSEVQLVQSGAEVKKPGSSVKVSCKASGYTFSSYVIHWVRQAPGQCLEWMGYINPYSDVIKYNEKFKGRVTLTSDKSTSTAYMELSSLRSEDTAVYYCARDSYYSYDGFAYWGQGTLVTVSSGGGGSGGGGSGGGGSGGGGSEIVLTQSPDFQSVTPKEKVTITCSASSSVSYMHWYQQKPDQSPKRLIYDTSKLASGVPSRFSGSGSGTDFTLTINSLEAEDAATYYCQQWSSNPPTFGCGTKVEIKGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 154 - protein, 214 aa, synthetic construct
DIQMTQSPSSLSASVGDRVTITCRASQDIGSNLNWLQQKPGKAIKRLIYATFGLDSGVPSRFSGSRSGTDFTLTISSLQPEDFATYYCQQYASFPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 155 - protein, 448 aa, synthetic construct; identical to SEQ ID NO: 152

SEQ ID NO: 156 - protein, 714 aa, synthetic construct
EVQLVQSGAEVKKPGASVKVSCKASGYTFTSYVIHWVRQAPGQGLEWMGYFNPYNDGTKYNEEFKGRVTLTSDKSTSTAYMELRSLRSDDTAVYYCARGGVRRYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSEVQLVQSGAEVKKPGSSVKVSCKASGYTFSSYVIHWVRQAPGQCLEWMGYINPYNDVIKYNEKFKGRVTLTSDKSTSTAYMELSSLRSEDTAVYYCARDSYYSYDGFAYWGQGTLVTVSSGGGGSGGGGSGGGGSGGGGSEIVLTQSPDFQSVTPKEKVTITCSASSSVSYMHWYQQKPDQSPKRLIYDTSKLASGVPSRFSGSGSGTDFTLTINSLEAEDAATYYCQQWSSNPPTFGCGTKVEIKGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 157 - protein, 446 aa, synthetic construct
EVQLVESGGGLVQPGRSLKLSCAASGFSFSNYGMAWVRQAATKGLEWVASISTGGGNTYYRDSVKGRFIVSRDNAKNTQYLQMDSLRSEDTATYYCTRHDRGGLYWGQGVMVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 158 - protein, 671 aa, synthetic construct
EVQLVESGGGLVQPGRSLKLSCAASGFSFSNYGMAWVRQAATKGLEWVASISTGGGNTYYRDSVKGRFIVSRDNAKNTQYLQMDSLRSEDTATYYCTRHDRGGLYWGQGVMVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 159 - protein, 219 aa, synthetic construct
DIVMTQAPLSVSVTPGESASISCRSSKSLLHSNGITYVYWYFQKPGKSPQVLIYRMSNLASGVPDRFSGSGSETDFTLKISRVEAEDVGIYHCGQLLENPYTFGAGTELELKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 160 - protein, 232 aa, synthetic construct
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 161 - protein, 446 aa, synthetic construct
EVQLVESGGGLVQPGRSLKLSCAASGFSFSNYGMAWVRQAATKGLEWVASISTGGGNTYYRDSVKGRFIVSRDNAKNTQYLQMDSLRSEDTATYYCTRHDRGGLYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 162 - protein, 720 aa, synthetic construct
EVQLVESGGGLVQPGRSLKLSCAASGFSFSNYGMAWVRQAATKGLEWVASISTGGGNTYYRDSVKGRFIVSRDNAKNTQYLQMDSLRSEDTATYYCTRHDRGGLYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSEVQLLESGGGLVQPGGSLRLSCAASGFTFSTYAMNWVRQAPGKCLEWVSRIRSKYNNYATYYADSVKGRFTISRDDSKNTLYLQMNSLRAEDTAVYYCVRHGNFGNSYVSWFAYWGQGTLVTVSSGGGGSGGGGSGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGCGTKLTVLGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 163 - protein, 219 aa, synthetic construct
DIVMTQAPLSVSVTPGESASISCRSSKSLLHSNGITYVYWYFQKPGKSPQVLIYRMSNLASGVPDRFSGSGSETDFTLKISRVEAEDVGIYHCGQLLENPYTFGAGTELELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 164 - protein, 448 aa, synthetic construct; identical to SEQ ID NO: 152

SEQ ID NO: 165 - protein, 722 aa, synthetic construct
EVQLVQSGAEVKKPGASVKVSCKASGYTFTSYVIHWVRQAPGQGLEWMGYFNPYNDGTKYNEEFKGRVTLTSDKSTSTAYMELRSLRSDDTAVYYCARGGVRRYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSEVQLLESGGGLVQPGGSLRLSCAASGFTFSTYAMNWVRQAPGKCLEWVSRIRSKYNNYATYYADSVKGRFTISRDDSKNTLYLQMNSLRAEDTAVYYCVRHGNFGNSYVSWFAYWGQGTLVTVSSGGGGSGGGGSGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGCGTKLTVLGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 166 - protein, 446 aa, synthetic construct; identical to SEQ ID NO: 157

SEQ ID NO: 167 - protein, 668 aa, synthetic construct
EVQLVESGGGLVQPGRSLKLSCAASGFSFSNYGMAWVRQAATKGLEWVASISTGGGNTYYRDSVKGRFIVSRDNAKNTQYLQMDSLRSEDTATYYCTRHDRGGLYWGQGVMVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSEIVLTQSPDFQSVTPKEKVTITCSASSSVSYMHWYQQKPDQSPKRLIYDTSKLASGVPSRFSGSGSGTDFTLTINSLEAEDAATYYCQQWSSNPPTFGGGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 168 - protein, 227 aa, synthetic construct
EVQLVQSGAEVKKPGSSVKVSCKASGYTFSSYVIHWVRQAPGQGLEWMGYINPYSDVIKYNEKFKGRVTLTSDKSTSTAYMELSSLRSEDTAVYYCARDSYYSYDGFAYWGQGTLVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 169 - protein, 446 aa, synthetic construct; identical to SEQ ID NO: 157

SEQ ID NO: 170 - protein, 668 aa, synthetic construct; identical to SEQ ID NO: 167

SEQ ID NO: 171 - protein, 227 aa, synthetic construct
EVQLVQSGAEVKKPGSSVKVSCKASGYTFSSYVIHWVRQAPGQGLEWMGYINPYNDVIKYNEKFKGRVTLTSDKSTSTAYMELSSLRSEDTAVYYCARDSYYSYDGFAYWGQGTLVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 172 - protein, 705 aa, synthetic construct
EVQLVESGGGLVQPGGSLRLSCAASGFSLSSSYWISWVRQAPGKGLEWIGSIDSGSVGITYYATWVKGRFTISRDNSKNTLYLQMNSLRAEDTAVYYCARHGDNWALDLWGQGTLVTVLEDLKNVFPPEVAVFEPSEAEISHTQKATLVCLATGFYPDHVELSWWVNGKEVHSGVCTDPQPLKEQPALQDSRYALSSRLRVSATFWQNPRNHFRCQVQFYGLSENDEWTQDRAKPVTQIVSAEAWGRGGGGSGGGGSQVQLVQSGAEVKKPGSSVKVSCKASGFAFTDYYIHWVRQAPGQGLEWMGWISPGNVNTKYNENFKGRVTITADKSTSTAYMELSSLRSEDTAVYYCARDGYSLYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 173 - protein, 475 aa, synthetic construct
EVQLVESGGGLVQPGGSLRLSCAASGFSLSSSYWISWVRQAPGKGLEWIGSIDSGSVGITYYATWVKGRFTISRDNSKNTLYLQMNSLRAEDTAVYYCARHGDNWALDLWGQGTLVTVLEDLKNVFPPEVAVFEPSEAEISHTQKATLVCLATGFYPDHVELSWWVNGKEVHSGVCTDPQPLKEQPALQDSRYALSSRLRVSATFWQNPRNHFRCQVQFYGLSENDEWTQDRAKPVTQIVSAEAWGRASDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 174 - protein, 273 aa, synthetic construct
DIVMTQSPDSLAVSLGERATINCKSSQSLLNSRTRKNYLAWYQQKPGQPPKLLIDIVMTQSPDSLAVSLGERATINCKSSQSLLNSRTRKNYLAWYQQKPGQPPKLLIYWASTRQSGVPDRFSGSGSGTDFTLTISSLQAEDVAVYYCTQSHTLRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 175 - protein, 205 aa, synthetic construct
DIQMTQSPSTLSASVGDRVTITCRASQSISSWLAWYQQKPGKAPKLLIYKASTLASGVPSRFSGSGSGTEFTLTISSLQPDDFATYYCQHGYIRGDLDNVFGGGTKVEIKPDIQNPDPAVYQLRDSKSSDKSVCLFTDFDSQTQVSQSKDSDVYITDKCVLDMRSMDFKSNSAVAWSQKSDFACANAFQNSIIPEDTFFPAPESS

SEQ ID NO: 176 - protein, 450 aa, synthetic construct
EVQLVESGGGLVQPGGSLRLSCAASGFSLSSSYWISWVRQAPGKGLEWIGSIDSGSVGITYYATWVKGRFTISRDNSKNTLYLQMNSLRAEDTAVYYCARHGDNWALDLWGQGTLVTVLEASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 177 - protein, 721 aa, synthetic construct
EVQLVESGGGLVQPGGSLRLSCAASGFSLSSSYWISWVRQAPGKGLEWIGSIDSGSVGITYYATWVKGRFTISRDNSKNTLYLQMNSLRAEDTAVYYCARHGDNWALDLWGQGTLVTVLEASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSQVQLVQSGAEVKKPGSSVKVSCKASGFAFTDYYIHWVRQAPGQCLEWMGWISPGNVNTKYNENFKGRVTITADKSTSTAYMELSSLRSEDTAVYYCARDGYSLYYFDYWGQGTLVTVSSGGGGSGGGGSGGGGSGGGGSDIVMTQSPDSLAVSLGERATINCKSSQSLLNSRTRKNYLAWYQQKPGQPPKLLIYWASTRQSGVPDRFSGSGSGTDFTLTISSLQAEDVAVYYCTQSHTLRTFGCGTKVEIKGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 178 - protein, 217 aa, synthetic construct
DIQMTQSPSTLSASVGDRVTITCRASQSISSWLAWYQQKPGKAPKLLIYKASTLASGVPSRFSGSGSGTEFTLTISSLQPDDFATYYCQHGYIRGDLDNVFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 179 - protein, 445 aa, synthetic construct
EVQLVESGGGLVQPGGSLRLSCAASGFDFSRYWMSWVRQAPGKGLVWIGEINPESSAINYAPSLKDRFTISRDNAKNTLYLQMNSLRAEDTAVYYCATSMTMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 180 - protein, 716 aa, synthetic construct
EVQLVESGGGLVQPGGSLRLSCAASGFDFSRYWMSWVRQAPGKGLVWIGEINPESSAINYAPSLKDRFTISRDNAKNTLYLQMNSLRAEDTAVYYCATSMTMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSQVQLVQSGAEVKKPGSSVKVSCKASGFAFTDYYIHWVRQAPGQCLEWMGWISPGNVNTKYNENFKGRVTITADKSTSTAYMELSSLRSEDTAVYYCARDGYSLYYFDYWGQGTLVTVSSGGGGSGGGGSGGGGSGGGGSDIVMTQSPDSLAVSLGERATINCKSSQSLLNSRTRKNYLAWYQQKPGQPPKLLIYWASTRQSGVPDRFSGSGSGTDFTLTISSLQAEDVAVYYCTQSHTLRTFGCGTKVEIKGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 181 - protein, 214 aa, synthetic construct
DIQMTQSPSSLSASVGDRVTITCKASQDIKSYLSWYQQKPGKAPKTLIYYATSLAGGVPSRFSGSGSGTDYTLTISSLQPEDFATYYCLQHGESPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 182 - protein, 5 aa, synthetic construct
DYYIH

SEQ ID NO: 183 - protein, 17 aa, synthetic construct
WISPGNVNTKYNENFKG

SEQ ID NO: 184 - protein, 10 aa, synthetic construct
DGYSLYYFDY

SEQ ID NO: 185 - protein, 17 aa, synthetic construct
KSSQSLLNSRTRKNYLA

SEQ ID NO: 186 - protein, 7 aa, synthetic construct
WASTRQS

SEQ ID NO: 187 - protein, 8 aa, synthetic construct
TQSHTLRT

SEQ ID NO: 188 - protein, 6 aa, synthetic construct
SSYWIS

SEQ ID NO: 189 - protein, 18 aa, synthetic construct
SIDSGSVGITYYATWVKG

SEQ ID NO: 190 - protein, 9 aa, synthetic construct
HGDNWALDL

SEQ ID NO: 191 - protein, 10 aa, synthetic construct
ASQSISSWLA

SEQ ID NO: 192 - protein, 7 aa, synthetic construct
KASTLAS

SEQ ID NO: 193 - protein, 12 aa, synthetic construct
QHGYIRGDLDNV

SEQ ID NO: 194 - protein, 120 aa, synthetic construct
EVQLVQSGAEVKKPGSSVKVSCKASGYTFSSYVIHWVRQAPGQGLEWMGYINPYSDVIKYNEKFKGRVTLTSDKSTSTAYMELSSLRSEDTAVYYCARDSYYSYDGFAYWGQGTLVTVSS

SEQ ID NO: 195 - protein, 120 aa, synthetic construct
EVQLVQSGAEVKKPGSSVKVSCKASGGTFSSYVIHWVRQAPGQCLEWMGYINPYNDVIKYNEKFKGRVTITADKSTSTAYMELSSLRSEDTAVYYCARDSYYSYDGFAYWGQGTLVTVSS

SEQ ID NO: 196 - protein, 120 aa, synthetic construct; identical to SEQ ID NO: 119

SEQ ID NO: 197 - protein, 120 aa, synthetic construct
EVQLVQSGAEVKKPGSSVKVSCKASGYTFSSYVIHWVRQAPGQCLEWIGYINPYNDVIKYNEKFKGRATLTSDKSTSTAYMELSSLRSEDTAVYYCARDSYYSYDGFAYWGQGTLVTVSS

SEQ ID NO: 198 - protein, 106 aa, synthetic construct
EIVLTQSPDFQSVTPKEKVTITCSASSSVSYMHWYQQKPDQSPKRLIYDTSKLASGVPSRFSGSGSGTDFTLTINSLEAEDAATYYCQQWSSNPPTFGCGTKVEIK

SEQ ID NO: 199 - protein, 106 aa, synthetic construct
EIVLTQSPDFQSVTPKEKVTITCSASSSVSYMHWYQQKPDQSPKRWIYDTSKLASGVPSRFSGSGSGTDYTLTINSLEAEDAATYYCQQWSSNPPTFGCGTKVEIK

SEQ ID NO: 200 - protein, 120 aa, synthetic construct; identical to SEQ ID NO: 138

SEQ ID NO: 201 - protein, 120 aa, synthetic construct; identical to SEQ ID NO: 124

SEQ ID NO: 202 - protein, 120 aa, synthetic construct
EVQLVQSGAEVKKPGSSVKVSCKASGYTFSSYVIHWVRQAPGQCLEWMGYINPYNDVIKYNEKFKGRVTLTSDKSTSTAYMELSSLRSEDTAVYYCARDSYYSYDAFAYWGQGTLVTVSS

SEQ ID NO: 203 - protein, 120 aa, synthetic construct
EVQLVQSGAEVKKPGSSVKVSCKASGYTFSSYVIHWVRQAPGQCLEWMGYINPGNDVIKYNEKFKGRVTLTSDKSTSTAYMELSSLRSEDTAVYYCARDSYYSYDGFAYWGQGTLVTVSS

SEQ ID NO: 204 - protein, 120 aa, synthetic construct; identical to SEQ ID NO: 125

SEQ ID NO: 205 - protein, 119 aa, synthetic construct
QVQLQQPGTELVKPGTSVKLSCKASGYTFTSYWMHWVKQRPGQCLEWIGNINPSNGATNYNEKFKSKATLTVDKSSSTAYMQLSSLTSEDSAVYYCARDRSSQYYFDYWGQGTTLTVSS

SEQ ID NO: 206 - protein, 112 aa, synthetic construct
DIVMSQSPSSLAVSAGEKVTMSCKSSQSLLNSRSRKNYLAWYQQKPGQSPKLLIYWASTRESGVPDRFTGSGSGTDFTLTISSVQAEDLAVYYCTQSYTLRTFGCGTKLEIK

SEQ ID NO: 207 - protein, 119 aa, synthetic construct
EVQLVQSGAEVKKPGSSVKVSCKASGYTFTSYWMHWVRQAPGQCLEWMGNINPSSGATNYNEKFKSRVTLTVDKSTSTAYMELSSLRSEDTAVYYCARDRSSQYYFDYWGQGTTVTVSS

SEQ ID NO: 208 - protein, 119 aa, synthetic construct
EVQLVQSGAEVKKPGSSVKVSCKASGYTFTRYWMHWVRQAPGQCLEWMGNINPSSGATNYNEKFKSRVTLTVDKSTSTAYMELSSLRSEDTAVYYCARDRSSQYYFDYWGQGTTVTVSS

SEQ ID NO: 209 - protein, 119 aa, synthetic construct
EVQLVQSGAEVKKPGSSVKVSCKASGYTFTSYWMHWVRQAPGQCLEWMGNINPRSGATNYNEKFKSRVTLTVDKSTSTAYMELSSLRSEDTAVYYCARDRSSQYYFDYWGQGTTVTVSS

SEQ ID NO: 210 - protein, 119 aa, synthetic construct
EVQLVQSGAEVKKPGSSVKVSCKASGYTFTSYWMHWVRQAPGQCLEWMGNINPSSGATNYNEKFKSRVTLTVDKSTSTAYMELSSLRSEDTAVYYCARDRSQQYYFDYWGQGTTVTVSS

SEQ ID NO: 211 - protein, 119 aa, synthetic construct
EVQLVQSGAEVKKPGSSVKVSCKASGYTFTSYWMHWVRQAPGQCLEWMGNINPSSGATNYNEKFKSRVTLTVDKSTSTAYMELSSLRSEDTAVYYCARDQSSQYYFDYWGQGTTVTVSS

SEQ ID NO: 212 - protein, 112 aa, synthetic construct
DIVMTQSPDSLAVSLGERATINCKSSQSLLNSRSRKNYLAWYQQKPGQPPKLLIYWASTRESGVPDRFSGSGSGTDFTLTISSLQAEDVAVYYCTQSYTLRTFGCGTKVEIK

SEQ ID NO: 213 - protein, 112 aa, synthetic construct
DIVMTQSPDSLAVSLGERATINCKSSQSLLNTRSRKNYLAWYQQKPGQPPKLLIYWASTRESGVPDRFSGSGSGTDFTLTISSLQAEDVAVYYCTQSYTLRTFGCGTKVEIK

SEQ ID NO: 214 - protein, 112 aa, synthetic construct
DIVMTQSPDSLAVSLGERATINCKSSQSLLNRRSRKNYLAWYQQKPGQPPKLLIYWASTRESGVPDRFSGSGSGTDFTLTISSLQAEDVAVYYCTQSYTLRTFGCGTKVEIK

SEQ ID NO: 215 - protein, 112 aa, synthetic construct
DIVMTQSPDSLAVSLGERATINCKSSQSLLNSRSRRNYLAWYQQKPGQPPKLLIYWASTRESGVPDRFSGSGSGTDFTLTISSLQAEDVAVYYCTQSYTLRTFGCGTKVEIK

SEQ ID NO: 216 - protein, 112 aa, synthetic construct
DIVMTQSPDSLAVSLGERATINCKSSQSLLYSRSRKNYLAWYQQKPGQPPKLLIYWASTRESGVPDRFSGSGSGTDFTLTISSLQAEDVAVYYCTQSYTLRTFGCGTKVEIK

SEQ ID NO: 217 - protein, 112 aa, synthetic construct
DIVMTQSPDSLAVSLGERATINCKSSQSLLNSRSKKNYLAWYQQKPGQPPKLLIYWASTRESGVPDRFSGSGSGTDFTLTISSLQAEDVAVYYCTQSYTLRTFGCGTKVEIK

SEQ ID NO: 218 - protein, 112 aa, synthetic construct
DIVMTQSPDSLAVSLGERATINCKSSQSLLNSASRKNYLAWYQQKPGQPPKLLIYWASTRESGVPDRFSGSGSGTDFTLTISSLQAEDVAVYYCTQSYTLRTFGCGTKVEIK

SEQ ID NO: 219 - protein, 112 aa, synthetic construct
DIVMTQSPDSLAVSLGERATINCKSSQSLLNSRSSKNYLAWYQQKPGQPPKLLIYWASTRESGVPDRFSGSGSGTDFTLTISSLQAEDVAVYYCTQSYTLRTFGCGTKVEIK

SEQ ID NO: 220 - protein, 112 aa, synthetic construct
DIVMTQSPDSLAVSLGERATINCKSSQSLLNSRSRKKYLAWYQQKPGQPPKLLIYWASTRESGVPDRFSGSGSGTDFTLTISSLQAEDVAVYYCTQSYTLRTFGCGTKVEIK

SEQ ID NO: 221 - protein, 120 aa, synthetic construct; identical to SEQ ID NO: 22

SEQ ID NO: 222 - protein, 120 aa, synthetic construct; identical to SEQ ID NO: 194

SEQ ID NO: 223 - protein, 120 aa, synthetic construct
EVQLVQSGAEVKKPGSSVKVSCKASGYTFSSYVIHWVRQAPGQGLEWMGYINPYNGVIKYNEKFKGRVTLTSDKSTSTAYMELSSLRSEDTAVYYCARDSYYSYDGFAYWGQGTLVTVSS

SEQ ID NO: 224 - protein, 120 aa, synthetic construct; identical to SEQ ID NO: 27

SEQ ID NO: 225 - protein, 120 aa, synthetic construct; identical to SEQ ID NO: 30

SEQ ID NO: 226 - protein, 120 aa, synthetic construct
EVQLVQSGAEVKKPGSSVKVSCKASGYTFSSYVIHWVRQAPGQGLEWMGYINPYNDVIKYNEKFKGRVTLTSDKSTSTAYMELSSLRSEDTAVYYCARESFSSYDGFAYWGQGTLVTVSS

SEQ ID NO: 227 - protein, 120 aa, synthetic construct
EVQLVQSGAEVKKPGSSVKVSCKASGYTFSSYVIHWVRQAPGQGLEWMGYINPGNGVIKYNEKFKGRVTLTSDKSTSTAYMELSSLRSEDTAVYYCARDSYSSYDGFAYWGQGTLVTVSS

SEQ ID NO: 228 - protein, 120 aa, synthetic construct
EVQLVQSGAEVKKPGSSVKVSCKASGYTFSSYVIHWVRQAPGQGLEWMGYINPYSDVIKYNEKFKGRVTLTSDKSTSTAYMELSSLRSEDTAVYYCARESFYSYDGFAYWGQGTLVTVSS

SEQ ID NO: 229 - protein, 120 aa, synthetic construct
EVQLVQSGAEVKKPGSSVKVSCKASGYTFSSYVIHWVRQAPGQGLEWMGYINPYNDVIKYNEKFKGRVTLTSDKSTSTAYMELSSLRSEDTAVYYCARDSYYSYEAFAYWGQGTLVTVSS

SEQ ID NO: 230 - protein, 120 aa, synthetic construct
EVQLVQSGAEVKKPGSSVKVSCKASGYTFSSYVIHWVRQAPGQGLEWMGYINPYNDVIKYNEKFKGRVTLTSDKSTSTAYMELSSLRSEDTAVYYCARDSYYSYRAFAYWGQGTLVTVSS

SEQ ID NO: 231 - protein, 120 aa, synthetic construct; identical to SEQ ID NO: 26

SEQ ID NO: 232 - protein, 120 aa, synthetic construct
EVQLVQSGAEVKKPGSSVKVSCKASGYTFSDYVIHWVRQAPGQGLEWMGYINPYNDVIKYNEKFKGRVTLTSDKSTSTAYMELSSLRSEDTAVYYCARDSYYSYDGFAYWGQGTLVTVSS

SEQ ID NO: 233 - protein, 106 aa, synthetic construct; identical to SEQ ID NO: 24

SEQ ID NO: 234 - protein, 106 aa, synthetic construct
EIVLTQSPDFQSVTPKEKVTITCSASSSVSYMHWYQQKPDQSPKRLIYDTSKLASGVPSRFSGSGSGTDFTLTINSLEAEDAATYYCQQWRSSPPTFGGGTKVEIK

SEQ ID NO: 235 - protein, 706 aa, synthetic construct
EVQLVESGGGLVQPGGSLRLSCAASGFSLSSSYWISWVRQAPGKGLEWIGSIDSGSVGITYYATWVKGRFTISRDNSKNTLYLQMNSLRAEDTAVYYCARHGDNWALDLWGQGTLVTVLEDLKNVFPPEVAVFEPSEAEISHTQKATLVCLATGFYPDHVELSWWVNGKEVHSGVCTDPQPLKEQPALQDSRYALSSRLRVSATFWQNPRNHFRCQVQFYGLSENDEWTQDRAKPVTQIVSAEAWGRGGGGSGGGGSEVQLVQSGAEVKKPGSSVKVSCKASGYTFSSYVIHWVRQAPGQGLEWMGYINPYSDVIKYNEKFKGRVTLTSDKSTSTAYMELSSLRSEDTAVYYCARDSYYSYDGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 236 - protein, 475 aa, synthetic construct; identical to SEQ ID NO: 173

SEQ ID NO: 237 - protein, 213 aa, synthetic construct
EIVLTQSPDFQSVTPKEKVTITCSASSSVSYMHWYQQKPDQSPKRLIYDTSKLASGVPSRFSGSGSGTDFTLTINSLEAEDAATYYCQQWSSNPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 238 - protein, 205 aa, synthetic construct; identical to SEQ ID NO: 175

SEQ ID NO: 239 - protein, 706 aa, synthetic construct
EVQLVESGGGLVQPGGSLRLSCAASGFSLSSSYWISWVRQAPGKGLEWIGSIDSGSVGITYYATWVKGRFTISRDNSKNTLYLQMNSLRAEDTAVYYCARHGDNWALDLWGQGTLVTVLEDLKNVFPPEVAVFEPSEAEISHTQKATLVCLATGFYPDHVELSWWVNGKEVHSGVCTDPQPLKEQPALQDSRYALSSRLRVSATFWQNPRNHFRCQVQFYGLSENDEWTQDRAKPVTQIVSAEAWGRGGGGSGGGGSEVQLVQSGAEVKKPGSSVKVSCKASGYTFSSYVIHWVRQAPGQGLEWMGYINPYNDVIKYNEKFKGRVTLTSDKSTSTAYMELSSLRSEDTAVYYCARDSYYSYDGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 240 - protein, 475 aa, synthetic construct; identical to SEQ ID NO: 173

SEQ ID NO: 241 - protein, 213 aa, synthetic construct; identical to SEQ ID NO: 237

SEQ ID NO: 242 - protein, 205 aa, synthetic construct; identical to SEQ ID NO: 175

SEQ ID NO: 243 - protein, 115 aa, synthetic construct
EVQLVESGGGLVQPGGSLRLSCAASGFDFSRYWMSWVRQAPGKGLVWIGEINPESSAINYAPSLKDRFTISRDNAKNTLYLQMNSLRAEDTAVYYCATSMTMDYWGQGTLVTVSS

SEQ ID NO: 244 - protein, 4 aa, synthetic construct
YWMS

SEQ ID NO: 245 - protein, 17 aa, synthetic construct
EINPESSAINYAPSLKD

SEQ ID NO: 246 - protein, 6 aa, synthetic construct
SMTMDY

SEQ ID NO: 247 - protein, 107 aa, synthetic construct
DIQMTQSPSSLSASVGDRVTITCKASQDIKSYLSWYQQKPGKAPKTLIYYATSLAGGVPSRFSGSGSGTDYTLTISSLQPEDFATYYCLQHGESPYTFGGGTKLEIK

SEQ ID NO: 248 - protein, 11 aa, synthetic construct
KASQDIKSYLS

SEQ ID NO: 249 - protein, 7 aa, synthetic construct
YATSLAG

SEQ ID NO: 250 - protein, 9 aa, synthetic construct
LQHGESPYT

SEQ ID NO: 251 - protein, 5 aa, synthetic construct
DYGVS

SEQ ID NO: 252 - protein, 16 aa, synthetic construct
VIWGSETTYYNSALKS

SEQ ID NO: 253 - protein, 12 aa, synthetic construct
HYYYGGSYAMDY

SEQ ID NO: 254 - protein, 11 aa, synthetic construct
RASQDISKYLN

SEQ ID NO: 255 - protein, 7 aa, synthetic construct
HTSRLHS

SEQ ID NO: 256 - protein, 9 aa, synthetic construct
QQGNTLPYT

SEQ ID NO: 257 - protein, 120 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 258 - protein, 107 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 259 - protein, 450 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 260 - protein, 716 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSEVQLVQSGAEVKKPGSSVKVSCKASGYTFSSYVIHWVRQAPGQCLEWMGYINPYNDVIKYNEKFKGRVTLTSDKSTSTAYMELSSLRSEDTAVYYCARDSFYSYDGFAYWGQGTLVTVSSGGGGSGGGGSGGGGSGGGGSEIVLTQSPDFQSVTPKEKVTITCSASSSVSYMHWYQQKPDQSPKRLIYDTSKLASGVPSRFSGSGSGTDFTLTINSLEAEDAATYYCQQWSSNPPTFGCGTKVEIKGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 261 - protein, 214 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 262 - protein, 213 aa, synthetic construct
QIVMSQSPAILSASPGEKVTMTCRARSSVSYIHWYQQKPGSSPKPWIYATSNLASGVPGRFSGSGSGTSYSLTITRVEAEDAATYYCQQWSSKPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 263 - protein, 716 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSEVQLVQSGAEVKKPGSSVKVSCKASGYTFSSYVIHWVRQAPGQCLEWMGYINPYSDVIKYNEKFKGRVTLTSDKSTSTAYMELSSLRSEDTAVYYCARDSYYSYDGFAYWGQGTLVTVSSGGGGSGGGGSGGGGSGGGGSEIVLTQSPDFQSVTPKEKVTITCSASSSVSYMHWYQQKPDQSPKRLIYDTSKLASGVPSRFSGSGSGTDFTLTINSLEAEDAATYYCQQWSSNPPTFGCGTKVEIKGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 264 - protein, 5 aa, synthetic construct
SYWMN

SEQ ID NO: 265 - protein, 17 aa, synthetic construct
QIWPGDGDTNYNGKFKG

SEQ ID NO: 266 - protein, 15 aa, synthetic construct
RETTTVGRYYYAMDY

SEQ ID NO: 267 - protein, 15 aa, synthetic construct
KASQSVDYDGDSYLN

SEQ ID NO: 268 - protein, 7 aa, synthetic construct
DASNLVS

SEQ ID NO: 269 - protein, 9 aa, synthetic construct
QQSTEDPWT

SEQ ID NO: 270 - protein, 124 aa, synthetic construct
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSS

SEQ ID NO: 271 - protein, 111 aa, synthetic construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK

SEQ ID NO: 272 - protein, 454 aa, synthetic construct
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 273 - protein, 720 aa, synthetic construct
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSEVQLVQSGAEVKKPGSSVKVSCKASGYTFSSYVIHWVRQAPGQCLEWMGYINPYNDVIKYNEKFKGRVTLTSDKSTSTAYMELSSLRSEDTAVYYCARDSFYSYDGFAYWGQGTLVTVSSGGGGSGGGGSGGGGSGGGGSEIVLTQSPDFQSVTPKEKVTITCSASSSVSYMHWYQQKPDQSPKRLIYDTSKLASGVPSRFSGSGSGTDFTLTINSLEAEDAATYYCQQWSSNPPTFGCGTKVEIKGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 274 - protein, 218 aa, synthetic construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 275 - protein, 720 aa, synthetic construct
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSEVQLVQSGAEVKKPGSSVKVSCKASGYTFSSYVIHWVRQAPGQCLEWMGYINPYSDVIKYNEKFKGRVTLTSDKSTSTAYMELSSLRSEDTAVYYCARDSYYSYDGFAYWGQGTLVTVSSGGGGSGGGGSGGGGSGGGGSEIVLTQSPDFQSVTPKEKVTITCSASSSVSYMHWYQQKPDQSPKRLIYDTSKLASGVPSRFSGSGSGTDFTLTINSLEAEDAATYYCQQWSSNPPTFGCGTKVEIKGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 276 - protein, 5 aa, synthetic construct
DYAMH

SEQ ID NO: 277 - protein, 17 aa, synthetic construct
TISWNSGSIGYADSVKG

SEQ ID NO: 278 - protein, 13 aa, synthetic construct
DIQYGNYYYGMDV

SEQ ID NO: 279 - protein, 11 aa, synthetic construct
RASQSVSSYLA

SEQ ID NO: 280 - protein, 7 aa, synthetic construct
DASNRAT

SEQ ID NO: 281 - protein, 9 aa, synthetic construct
QQRSNWPIT

SEQ ID NO: 282 - protein, 122 aa, synthetic construct
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSS

SEQ ID NO: 283 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIK

SEQ ID NO: 284 - protein, 452 aa, synthetic construct
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 285 - protein, 718 aa, synthetic construct
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSEVQLVQSGAEVKKPGSSVKVSCKASGYTFSSYVIHWVRQAPGQCLEWMGYINPYNDVIKYNEKFKGRVTLTSDKSTSTAYMELSSLRSEDTAVYYCARDSFYSYDGFAYWGQGTLVTVSSGGGGSGGGGSGGGGSGGGGSEIVLTQSPDFQSVTPKEKVTITCSASSSVSYMHWYQQKPDQSPKRLIYDTSKLASGVPSRFSGSGSGTDFTLTINSLEAEDAATYYCQQWSSNPPTFGCGTKVEIKGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 286 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 287 - protein, 718 aa, synthetic construct
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSEVQLVQSGAEVKKPGSSVKVSCKASGYTFSSYVIHWVRQAPGQCLEWMGYINPYSDVIKYNEKFKGRVTLTSDKSTSTAYMELSSLRSEDTAVYYCARDSYYSYDGFAYWGQGTLVTVSSGGGGSGGGGSGGGGSGGGGSEIVLTQSPDFQSVTPKEKVTITCSASSSVSYMHWYQQKPDQSPKRLIYDTSKLASGVPSRFSGSGSGTDFTLTINSLEAEDAATYYCQQWSSNPPTFGCGTKVEIKGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 288 - protein, 5 aa, synthetic construct
SYNMH

SEQ ID NO: 289 - protein, 17 aa, synthetic construct
AIYPGNGDTSYNQKFKG

SEQ ID NO: 290 - protein, 12 aa, synthetic construct
STYYGGDWYFNV

SEQ ID NO: 291 - protein, 10 aa, synthetic construct
RASSSVSYIH

SEQ ID NO: 292 - protein, 7 aa, synthetic construct
ATSNLAS

SEQ ID NO: 293 - protein, 9 aa, synthetic construct
QQWTSNPPT

SEQ ID NO: 294 - protein, 121 aa, synthetic construct [sequence omitted]

SEQ ID NO: 295 - protein, 106 aa, synthetic construct [sequence omitted]

SEQ ID NO: 296 - protein, 451 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 297 - protein, 717 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSEVQLVQSGAEVKKPGSSVKVSCKASGYTFSSYVIHWVRQAPGQCLEWMGYINPYNDVIKYNEKFKGRVTLTSDKSTSTAYMELSSLRSEDTAVYYCARDSFYSYDGFAYWGQGTLVTVSSGGGGSGGGGSGGGGSGGGGSEIVLTQSPDFQSVTPKEKVTITCSASSSVSYMHWYQQKPDQSPKRLIYDTSKLASGVPSRFSGSGSGTDFTLTINSLEAEDAATYYCQQWSSNPPTFGCGTKVEIKGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 298 - protein, 213 aa, synthetic construct
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 299 - protein, 717 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSEVQLVQSGAEVKKPGSSVKVSCKASGYTFSSYVIHWVRQAPGQCLEWMGYINPYSDVIKYNEKFKGRVTLTSDKSTSTAYMELSSLRSEDTAVYYCARDSYYSYDGFAYWGQGTLVTVSSGGGGSGGGGSGGGGSGGGGSEIVLTQSPDFQSVTPKEKVTITCSASSSVSYMHWYQQKPDQSPKRLIYDTSKLASGVPSRFSGSGSGTDFTLTINSLEAEDAATYYCQQWSSNPPTFGCGTKVEIKGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 300 - protein, 720 aa, synthetic construct
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSEVQLVQSGAEVKKPGSSVKVSCKASGYTFSSYVIHWVRQAPGQCLEWMGYINPYNDVIKYNEKFKGRVTLTSDKSTSTAYMELSSLRSEDTAVYYCARDSYYSYRGFAYWGQGTLVTVSSGGGGSGGGGSGGGGSGGGGSEIVLTQSPDFQSVTPKEKVTITCSASSSVSYMHWYQQKPDQSPKRLIYDTSKLASGVPSRFSGSGSGTDFTLTINSLEAEDAATYYCQQWSSNPPTFGCGTKVEIKGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 301 - protein, 5 aa, synthetic construct
FYYMH

SEQ ID NO: 302 - protein, 17 aa, synthetic construct
RIDPEDESTKYSEKFKN

SEQ ID NO: 303 - protein, 7 aa, synthetic construct
GGYYFDY

SEQ ID NO: 304 - protein, 11 aa, synthetic construct
QASEDIYSGLA

SEQ ID NO: 305 - protein, 7 aa, synthetic construct
GASDLQD

SEQ ID NO: 306 - protein, 9 aa, synthetic construct
QQGLTYPRT

SEQ ID NO: 307 - protein, 5 aa, synthetic construct
GYGMH

SEQ ID NO: 308 - protein, 17 aa, synthetic construct
YITSSSINIKYADAVKG

SEQ ID NO: 309 - protein, 7 aa, synthetic construct
FDWDKNY

SEQ ID NO: 310 - protein, 11 aa, synthetic construct
QASQDISNYLN

SEQ ID NO: 311 - protein, 7 aa, synthetic construct
YTNKLAD

SEQ ID NO: 312 - protein, 9 aa, synthetic construct
QQYYNYPWT

SEQ ID NO: 313 - protein, 116 aa, synthetic construct
EVQLQQSGAELVRPGTSVKLSCKVSGDTITFYYMHFVKQRPGQGLEWIGRIDPEDESTKYSEKFKNKATLTADTSSNTAYLKLSSLTSEDTATYFCIYGGYYFDYWGQGVMVTVSS

SEQ ID NO: 314 - protein, 107 aa, synthetic construct
DIQMTQSPASLSTSLGETVTIQCQASEDIYSGLAWYQQKPGKSPQLLIYGASDLQDGVPSRFSGSGSGTQYSLKITSMQTEDEGVYFCQQGLTYPRTFGGGTKLELK

SEQ ID NO: 315 - protein, 116 aa, synthetic construct
EVQLVESGGGLVQPGKSLKLSCEASGFTFSGYGMHWVRQAPGRCLESVAYITSSSINIKYADAVKGRFTVSRDNAKNLLFLQMNILKSEDTAMYYCARFDWDKNYWGQGTMVTVSS

SEQ ID NO: 316 - protein, 107 aa, synthetic construct
DIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGCGTKLEIK

SEQ ID NO: 317 - protein, 446 aa, synthetic construct
EVQLQQSGAELVRPGTSVKLSCKVSGDTITFYYMHFVKQRPGQGLEWIGRIDPEDESTKYSEKFKNKATLTADTSSNTAYLKLSSLTSEDTATYFCIYGGYYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 318 - protein, 709 aa, synthetic construct
EVQLQQSGAELVRPGTSVKLSCKVSGDTITFYYMHFVKQRPGQGLEWIGRIDPEDESTKYSEKFKNKATLTADTSSNTAYLKLSSLTSEDTATYFCIYGGYYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSEVQLVESGGGLVQPGKSLKLSCEASGFTFSGYGMHWVRQAPGRCLESVAYITSSSINIKYADAVKGRFTVSRDNAKNLLFLQMNILKSEDTAMYYCARFDWDKNYWGQGTMVTVSSGGGGSGGGGSGGGGSGGGGSDIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGCGTKLEIKGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 319 - protein, 214 aa, synthetic construct
DIQMTQSPASLSTSLGETVTIQCQASEDIYSGLAWYQQKPGKSPQLLIYGASDLQDGVPSRFSGSGSGTQYSLKITSMQTEDEGVYFCQQGLTYPRTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 320 - protein, 5 aa, synthetic construct
SYWMH

SEQ ID NO: 321 - protein, 17 aa, synthetic construct
VIDPSDNYTKYNQKFKG

SEQ ID NO: 322 - protein, 12 aa, synthetic construct
EGYYGSSPWFAY

SEQ ID NO: 323 - protein, 10 aa, synthetic construct
RARSSVSYIH

SEQ ID NO: 324 - protein, 7 aa, synthetic construct
ATSNLAS

SEQ ID NO: 325 - protein, 9 aa, synthetic construct
QQWSSKPPT

SEQ ID NO: 326 - protein, 121 aa, synthetic construct
QVQLQQPGAELVRPGTSVKLSCKASGYTFTSYWMHWIKQRPGQGLEWIGVIDPSDNYTKYNQKFKGKATLTVDTSSSTAYMQLSSLTSEDSAVYFCAREGYYGSSPWFAYWGQGTLVTVSS

SEQ ID NO: 327 - protein, 106 aa, synthetic construct
QIVMSQSPAILSASPGEKVTMTCRARSSVSYIHWYQQKPGSSPKPWIYATSNLASGVPGRFSGSGSGTSYSLTITRVEAEDAATYYCQQWSSKPPTFGGGTKLEIK

SEQ ID NO: 328 - protein, 451 aa, synthetic construct
QVQLQQPGAELVRPGTSVKLSCKASGYTFTSYWMHWIKQRPGQGLEWIGVIDPSDNYTKYNQKFKGKATLTVDTSSSTAYMQLSSLTSEDSAVYFCAREGYYGSSPWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 329 - protein, 714 aa, synthetic construct
QVQLQQPGAELVRPGTSVKLSCKASGYTFTSYWMHWIKQRPGQGLEWIGVIDPSDNYTKYNQKFKGKATLTVDTSSSTAYMQLSSLTSEDSAVYFCAREGYYGSSPWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSEVQLVESGGGLVQPGKSLKLSCEASGFTFSGYGMHWVRQAPGRCLESVAYITSSSINIKYADAVKGRFTVSRDNAKNLLFLQMNILKSEDTAMYYCARFDWDKNYWGQGTMVTVSSGGGGSGGGGSGGGGSGGGGSDIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGCGTKLEIKGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 330 - protein, 213 aa, synthetic construct
QIVMSQSPAILSASPGEKVTMTCRARSSVSYIHWYQQKPGSSPKPWIYATSNLASGVPGRFSGSGSGTSYSLTITRVEAEDAATYYCQQWSSKPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 331 - protein, 446 aa, synthetic construct
EVQLQQSGAELVRPGTSVKLSCKVSGDTITFYYMHFVKQRPGQGLEWIGRIDPEDESTKYSEKFKNKATLTADTSSNTAYLKLSSLTSEDTATYFCIYGGYYFDYWGQGVMVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPDVFIFPPKIKDVLMISLSPIVTCVVVDVSEDDPDVQISWFVNNVEVHTAQTQTHREDYNSTLRVVSALPIQHQDWMSGKEFKCKVNNKDLPLPEERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 332 - protein, 214 aa, synthetic construct
DIQMTQSPASLSTSLGETVTIQCQASEDIYSGLAWYQQKPGKSPQLLIYGASDLQDGVPSRFSGSGSGTQYSLKITSMQTEDEGVYFCQQGLTYPRTFGGGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 333 - protein, 451 aa, synthetic construct
QVQLQQPGAELVRPGTSVKLSCKASGYTFTSYWMHWIKQRPGQGLEWIGVIDPSDNYTKYNQKFKGKATLTVDTSSSTAYMQLSSLTSEDSAVYFCAREGYYGSSPWFAYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPDVFIFPPKIKDVLMISLSPIVTCVVVDVSEDDPDVQISWFVNNVEVHTAQTQTHREDYNSTLRVVSALPIQHQDWMSGKEFKCKVNNKDLPLPEERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK